(19) United States
(12) Patent Application Publication
(10) Pub. No.: US 2025/0145729 A1
(43) Pub. Date: May 8, 2025

(54) POLYPEPTIDE ENGINEERING, LIBRARIES, AND ENGINEERED CD98 HEAVY CHAIN AND TRANSFERRIN RECEPTOR BINDING POLYPEPTIDES

(71) Applicant: DENALI THERAPEUTICS INC., South San Francisco, CA (US)

(72) Inventors: Padma Akkapeddi, South San Francisco, CA (US); Gerald Maxwell Cherf, South San Francisco, CA (US); Kylie S. Chew, South San Francisco, CA (US); Mark S. Dennis, South San Francisco, CA (US); Zhenyu Gu, South San Francisco, CA (US); Mihalis S. Kariolis, South San Francisco, CA (US); Hai L. Tran, South San Francisco, CA (US); Robert C. Wells, South San Francisco, CA (US); Joy Yu Zuchero, South San Francisco, CA (US)

(73) Assignee: DENALI THERAPEUTICS INC., South San Francisco, CA (US)

(21) Appl. No.: 18/716,489

(22) PCT Filed: Dec. 16, 2022
(86) PCT No.: PCT/US2022/053220

§ 371 Date: Jun. 4, 2024

Related U.S. Application Data
(60) Provisional application No. 63/291,161, filed on Dec. 17, 2021.
(60) Provisional application No. 63/423,418, filed on Nov. 7, 2022.

(51) Int. Cl. C07K 16/28; A61K 39/00

(52) U.S. Cl. CPC C07K 16/2896; A61K 2039/505; C07K 2317/33; C07K 2317/35; C07K 2317/52; C07K 2317/55; C07K 2317/92; C07K 2317/94

(57) ABSTRACT

The present disclosure includes engineering methods and polypeptide libraries that are useful for introducing non-native binding sites into polypeptides. Also provided herein are polypeptides that bind to a CD98hc or transferrin receptor (TfR) protein, methods of generating such polypeptides, and methods of using the polypeptides to target a composition across the blood-brain barrier or to a CD98hc-expressing or TfR-expressing cell.

### CROSS-REFERENCE TO RELATED APPLICATIONS

The present application is a 371 national stage application of PCT/US2022/053220, filed on Dec. 16, 2022, which claims priority to U.S. Provisional Patent Application No. 63/291,161, filed on Dec. 17, 2021, and U.S. Provisional Patent Application No. 63/423,418, filed on Nov. 7, 2022, the disclosures of which are incorporated herein by reference in their entirety for all purposes.

### BACKGROUND

Various techniques have been developed that engineer a protein to bind to a target that it does not normally bind. For example, libraries can be generated to screen for engineered proteins with desired binding or enzymatic activity.

### BRIEF SUMMARY

We have developed several approaches for discovery of polypeptides with novel binding sites, particularly those that include a beta-sheet part of the binding site. These approaches include the development of beta-sheet libraries, and libraries that employ a “limited liability” approach to reduce the frequency of amino acids that can produce proteins with undesirable characteristics. We have used these types of libraries to discover polypeptides that bind to targets such as the CD98 heavy chain (CD98hc) and the transferrin receptor (TfR), as described in detail below. We have also developed methods of delivery (e.g., across the blood-brain barrier) using CD98hc polypeptides, particularly to extracellular targets in the brain.

In one aspect, the disclosure provides a method of engineering a non-native binding site into a polypeptide, the method comprising:
(a) generating a library of polypeptides, wherein at least a portion of the polypeptides includes at least seven randomized positions, wherein 10⁻⁶⁰% of the randomized positions have diversity limited to exclude one or more of the following amino acids: Cys, Trp, Met, Arg, or Gly, but include at least eight amino acids at each position;
(b) contacting the library with a target protein;
(c) selecting library members that bind to the target protein; and
(d) isolating the selected library members, thereby engineering a non-native binding site into the polypeptide.

In some embodiments, the method comprises repeating steps (b)-(d) using the library members isolated from first step (d). In some embodiments, the library includes at least 8, 9, 10, 11, 12, 13, 14, 15, 16, 17, 18, 19, 20, or more randomized positions. In some embodiments, the primary amino acid sequence of each polypeptide comprises positions with limited diversity that are separated by positions without limited diversity.

In some embodiments, each polypeptide includes a beta-sheet and at least three of the randomized positions are present within a single beta-sheet. In certain embodiments, at least three of the randomized positions are present within at least two beta-strands that form the beta-sheet. In certain embodiments, at least three of the randomized positions are present within at least one beta-strand that forms the beta-sheet. In some embodiments, at least three of the randomized positions form a surface on one side of the beta-sheet. In particular embodiments, at least three of the randomized positions are surface exposed. In some embodiments, the beta-sheet includes at least one position with limited diversity. In certain embodiments, the beta-sheet includes at least two positions with limited diversity. In certain embodiments, the at least two positions with limited diversity are separated by a position without limited diversity. In some embodiments, the beta-sheet includes at least two positions without limited diversity. In certain embodiments, the at least two positions without limited diversity are separated by a position with limited diversity. In particular embodiments, the separation is relative to the primary amino acid sequence of the polypeptide or is relative to the spatial three-dimensional positioning of the amino acid in the protein structure.

In some embodiments, the positions with limited diversity are coded for by degenerate codons. In certain embodiments, at least one of the degenerate codons is NHK. In some embodiments, the positions without limited diversity are coded for by the degenerate codon NNK.

In some embodiments, the polypeptide contains an immunoglobulin-like fold. In certain embodiments, the polypeptide includes an immunoglobulin (IgG) domain. In certain embodiments, the IgG domain is from an IgG, IgA, IgE, IgM, or IgD family. In certain embodiments, the IgG domain is selected is from an IgG1, IgG2, IgG3, or IgG4 molecule. In particular embodiments, the IgG domain includes a VH, CH1, CH2, CH3, VL or CL domain. In some embodiments, the randomized positions are surface accessible. In particular embodiments, the randomized positions are selected from any of those listed in Table 1B. In certain embodiments, the polypeptide includes a fibronectin or any other protein scaffold described herein.

In another aspect, the disclosure provides a library of polypeptides, wherein at least a portion of the polypeptides includes at least seven randomized positions, wherein 10⁻⁶⁰% of the randomized positions have diversity limited to exclude one or more of the following amino acids: Cys, Trp, Met, Arg, or Gly, but include at least eight amino acids at each position.

In some embodiments, the library includes at least 8, 9, 10, 11, 12, 13, 14, 15, 16, 17, 18, 19, 20, or more randomized positions. In some embodiments, the primary amino acid sequence of each polypeptide comprises positions with limited diversity separated by positions without limited diversity.

In some embodiments, each polypeptide includes a beta-sheet and at least three of the randomized positions are present within a single beta-sheet. In certain embodiments, at least three of the randomized positions are present within at least two beta-strands that form the beta-sheet. In certain embodiments, at least three of the randomized positions are present within at least one beta-strand that forms the beta-sheet. In some embodiments, at least three of the randomized positions form a surface on one side of the beta-sheet. In particular embodiments, at least three of the randomized positions are surface exposed. In some embodiments, the beta-sheet includes at least one position with limited diversity. In certain embodiments, the beta-sheet includes at least two positions with limited diversity. In certain embodiments, the at least two positions with limited diversity are separated by a position without limited diversity. In particular embodiments, the separation is relative to the primary sequence of the polypeptide or is relative to the spatial three-dimensional positioning of the amino acids in the protein structure. In some embodiments, the beta-sheet includes at least two positions without limited diversity. In certain embodiments, the at least two positions without limited diversity are separated by a position with limited diversity. In particular embodiments, the separation is relative to the primary sequence of the polypeptide or is relative to the spatial three-dimensional positioning of the amino acid in the protein structure.

In some embodiments, the polypeptide contains an immunoglobulin-like fold. In certain embodiments, the polypeptide includes an immunoglobulin (IgG) domain. In certain embodiments, the IgG domain is from an IgG, IgA, IgE, IgM, or IgD family. In certain embodiments, the IgG domain is selected is from an IgG1, IgG2, IgG3, or IgG4 molecule. In particular embodiments, the IgG domain includes a VH, CH1, CH2, CH3, VL or CL domain. In some embodiments, the randomized positions are surface accessible. In particular embodiments, the randomized positions are selected from any of those listed in Table 1B. In certain embodiments, the polypeptide includes a fibronectin or any other protein scaffold described herein.

In another aspect, the disclosure provides a polypeptide comprising a constant domain or non-CDR portion of a variable domain of an immunoglobulin having at least three modified positions in a beta-sheet, wherein:
(i) the modified positions are in at least two beta-strands forming the beta-sheet;
(ii) the modified positions form at least part of a binding site that is capable of binding to an antigen; and
(iii) the beta-sheet does not bind to the antigen without the modified positions.

In some embodiments, the constant domain comprises an Fc polypeptide. In some embodiments, the at least two beta-strands are selected from the group consisting of: amino acid positions 124-128, 139-147, 155-157, 179-178, 199-203, 208-214, 239-243, 258-265, 274-278, 301-307, 319-324, 332-336, 347-351, 363-372, 378-383, 391-393, 406-412, 423-428, and 437-441, wherein the positions are determined according to EU numbering. In certain embodiments, the positions are surface accessible. In particular embodiments, the positions are selected from those listed in Table 1B.

In some embodiments, the modified positions form a contiguous surface on the beta-sheet. In some embodiments, the modified positions are surface accessible residues. In certain embodiments, the surface accessible residues are selected from the group consisting of: amino acid positions 347, 349, 351, 362, 364, 366, 368, 370, 378, 380, 382, 405, 407, 409, 411, 424, 426, 428, 436, 438, and 440, wherein the positions are determined according to EU numbering. In certain embodiments, the surface accessible residues are selected from the group consisting of: amino acid positions 347, 362, 378, 380, 382, 411, 424, 426, 428, 436, 438, and 440, wherein the positions are determined according to EU numbering.

In some embodiments, the modified positions comprise three, four, five, six, or seven amino acid substitutions in a set of amino acid positions comprising 380, 382, 383, 424, 426, 438, and 440, wherein the positions are determined according to EU numbering. In some embodiments, the modified positions comprise three, four, five, six, seven, eight, nine, ten, or eleven amino acid substitutions in a set of amino acid positions comprising 378, 380, 382, 383, 422, 424, 426, 428, 438, 440, and 442, wherein the positions are determined according to EU numbering.

In some embodiments, the binding site includes one or more modified positions in at least one loop region. In certain embodiments, the one or more modified positions in at least one loop region are selected from the group consisting of: amino acid positions 387 and 422, wherein the positions are determined according to EU numbering. In particular embodiments, the loop region connects the two beta-strands.

In another aspect, the disclosure provides a method of introducing a non-native binding site into a constant domain or non-CDR region of a variable domain of an immunoglobulin, the method comprising:
(a) generating a polynucleotide library that encodes an immunoglobulin sequence having at least three modified positions in a beta-sheet, wherein the library is randomized at codons that code for amino acids at the modified positions, wherein the modified positions are in at least two beta-strands forming the beta-sheet;
(b) expressing the library to produce a library of sequence variants;
(c) contacting the sequence variants with a target protein; and
(d) isolating sequence variants that bind to the target protein, thereby introducing a non-native binding site into a constant domain or non-CDR region of a variable domain of the immunoglobulin.

In some embodiments, the immunoglobulin sequence comprises an Fc polypeptide. In some embodiments, the at least two beta-strands are selected from the group consisting of: amino acid positions 239-243, 258-265, 274-278, 301-307, 319-324, 332-336, 347-351, 363-372, 378-383, 391-393, 406-412, 423-428, and 437-441, wherein the positions are determined according to EU numbering.

In some embodiments, the modified positions form a contiguous surface on the beta-sheet. In some embodiments, the modified positions are surface accessible residues. In certain embodiments, the surface accessible residues are selected from the group consisting of: amino acid positions 347, 349, 351, 362, 364, 366, 368, 370, 378, 380, 382, 405, 407, 409, 411, 424, 426, 428, 436, 438, and 440, wherein the positions are determined according to EU numbering. In certain embodiments, the surface accessible residues are selected from the group consisting of: amino acid positions 347, 362, 378, 380, 382, 411, 424, 426, 428, 436, 438, and 440, wherein the positions are determined according to EU numbering.

In some embodiments, the modified positions comprise three, four, five, six, or seven amino acid substitutions in a set of amino acid positions comprising 380, 382, 383, 424, 426, 438, and 440, wherein the positions are determined according to EU numbering. In some embodiments, the modified positions comprise three, four, five, six, seven, eight, nine, ten, or eleven amino acid substitutions in a set of amino acid positions comprising 378, 380, 382, 383, 422, 424, 426, 428, 438, 440, and 442, wherein the positions are determined according to EU numbering.

In some embodiments, the binding site includes one or more modified positions in at least one loop region. In certain embodiments, the one or more modified positions in at least one loop region are selected from the group consisting of: amino acid positions 387 and 422, wherein the positions are determined according to EU numbering. In particular embodiments, the loop region connects the two beta-strands.

In another aspect, the disclosure provides a library of immunoglobulin variants comprising at least ten members, wherein the variants each comprise at least three modified positions in a beta-sheet that forms part of a constant or non-CDR variable domain of the immunoglobulin, wherein the modified positions are in at least two-beta strands that form the beta-sheet.

In some embodiments, the library of immunoglobulin variants comprises at least 10², 10³, 10⁴, 10⁵, 10⁶, 10⁷, 10⁸, 10⁹, 10¹⁰, 10¹¹, 10¹², or more members. In some embodiments, the library is generated from a collection of coding polynucleotides that code for at least seven randomized amino acid positions, wherein 10⁻⁶⁰% of the randomized positions have diversity limited to exclude one or more of the following amino acids: Cys, Trp, Met, Arg, or Gly, but include at least eight amino acids at each position. In certain embodiments, at least one of diversity-limited positions does not code for tryptophan or cysteine. In certain embodiments, at least two diversity-limited positions do not code for tryptophan or cysteine. In particular embodiments, the at least two diversity-limited positions do not code for tryptophan, cysteine, or arginine.

In some embodiments, the diversity-limited positions are coded for by degenerate codons. In certain embodiments, at least one diversity-limited position is coded for by an NHK codon. In particular embodiments, the NHK codons are not adjacent to each other in the primary amino acid sequence or are not adjacent to each other in the three-dimensional protein structure. In certain embodiments, the NHK codons are present in an alternating pattern with one or more NNK codons.

In some embodiments, the disclosure provides a library of polynucleotides encoding the immunoglobulin variants from the library described herein.

In another aspect, the disclosure provides a method of engineering a non-native binding site to a transferrin receptor (TfR) or to a CD98hc protein into a polypeptide, the method comprising:
(a) generating a library of polypeptides, wherein at least a portion of the polypeptides includes at least seven randomized positions, wherein 10⁻⁶⁰% of the randomized positions have diversity limited to exclude one or more of the following amino acids: Cys, Trp, Met, Arg, or Gly, but include at least eight amino acids at each position;
(b) contacting the library with a target protein;
(c) selecting library members that bind to the target protein; and
(d) isolating the selected library members, thereby engineering a non-native binding site to TfR or CD98hc into the polypeptide.

In some embodiments of this aspect, the method comprises repeating steps (b)-(d) using the library members isolated from first step (d).

In some embodiments, the library includes at least 8, 9, 10, 11, 12, 13, 14, 15, 16, 17, 18, 19, 20, or more randomized positions.

In some embodiments, the primary amino acid sequence of each polypeptide comprises positions with limited diversity that are separated by positions without limited diversity. In particular embodiments, each polypeptide includes a beta-sheet and at least three of the randomized positions are present within a single beta-sheet. In certain embodiments, at least three of the randomized positions are present within at least two beta-strands that form the beta-sheet. In particular embodiments, at least three of the randomized positions are present within at least one beta-strand that forms the beta-sheet. In particular embodiments, at least three of the randomized positions form a surface on one side of the beta-sheet. In particular embodiments, at least three of the randomized positions are surface exposed.

In some embodiments of this aspect, the beta-sheet includes at least one position with limited diversity. In some embodiments, the beta-sheet includes at least two positions with limited diversity. In some embodiments, the at least two positions with limited diversity are separated by a position without limited diversity.

In some embodiments, the beta-sheet includes at least two positions without limited diversity. In particular embodiments, the at least two positions without limited diversity are separated by a position with limited diversity.

In some embodiments, the separation is relative to the primary amino acid sequence of the polypeptide or is relative to the spatial three-dimensional positioning of the amino acid in the protein structure.

In some embodiments, the positions with limited diversity are coded for by degenerate codons. In certain embodiments, at least one of the degenerate codons is NHK. In certain embodiments, the positions without limited diversity are coded for by the degenerate codon NNK.

In some embodiments of this aspect, the polypeptide contains an immunoglobulin-like fold. In some embodiments, the polypeptide includes an immunoglobulin (IgG) domain. In certain embodiments, the IgG domain is from an IgG, IgA, IgE, IgM, or IgD family. In certain embodiments, the IgG domain is selected is from an IgG1, and IgG2, and IgG3, or IgG4 molecule. In certain embodiments, the IgG domain includes a VH, CH1, CH2, CH3, VL or CL domain.

In some embodiments, the randomized positions are surface accessible. In certain embodiments, the randomized positions are selected from any of those listed in Table 1B. In particular embodiments, the polypeptide includes a fibronectin or any other protein scaffold described herein.

In another aspect, the disclosure provides a polypeptide having at least three modified positions in a beta-sheet portion, wherein:
(i) the modified positions are in at least two beta-strands forming the beta-sheet;
(ii) the modified positions form at least part of a binding site that is capable of binding to CD98hc; and
(iii) the beta-sheet does not bind to the antigen without the modified positions.

In some embodiments of this aspect, the polypeptide comprises at least 4 or 5 modified positions in the beta-sheet. In some embodiments, the polypeptide comprises at least seven modified positions that form at least part of a binding site capable of binding CD98hc. In some embodiments, the polypeptide contains an immunoglobulin-like fold. In certain embodiments, the polypeptide includes an immunoglobulin (IgG) domain. In certain embodiments, the IgG domain is from an IgG, IgA, IgE, IgM, or IgD family. In particular embodiments, the IgG domain is selected is from an IgG1, and IgG2, and IgG3, or IgG4 molecule. In certain embodiments, the IgG domain includes a VH, CH1, CH2, CH3, VL or CL domain.

In some embodiments, the modified positions are surface accessible. In some embodiments, the modified positions are selected from any of those listed in Table 1B. In certain embodiments, the polypeptide includes a fibronectin or any other protein scaffold described herein.

In another aspect, the disclosure provides a polypeptide comprising a constant domain or non-CDR portion of a variable domain of an immunoglobulin having at least three modified positions in a beta-sheet, wherein:
(i) the modified positions are in at least two beta-strands forming the beta-sheet;
(ii) the modified positions form at least part of a binding site that is capable of binding to a TfR or to a CD98hc protein; and
(iii) the beta-sheet does not bind to the antigen without the modified positions.

In some embodiments of this aspect, the constant domain comprises an Fc polypeptide.

In some embodiments, the at least two beta-strands are selected from the group consisting of: amino acid positions 124-128, 139-147, 155-157, 179-178, 199-203, 208-214, 239-243, 258-265, 274-278, 301-307, 319-324, 332-336, 347-351, 363-372, 378-383, 391-393, 406-412, 423-428, and 437-441, wherein the positions are determined according to EU numbering. In some embodiments, the positions are surface accessible. In certain embodiments, the positions are selected from those listed in Table 1B.

In some embodiments, the modified positions form a contiguous surface on the beta-sheet.

In some embodiments, the modified positions are surface accessible residues. In certain embodiments, the surface accessible residues are selected from the group consisting of: amino acid positions 347, 349, 351, 362, 364, 366, 368, 370, 378, 380, 382, 405, 407, 409, 411, 424, 426, 428, 436, 438, and 440, wherein the positions are determined according to EU numbering. In certain embodiments, the surface accessible residues are selected from the group consisting of: amino acid positions 347, 362, 378, 380, 382, 411, 424, 426, 428, 436, 438, and 440, wherein the positions are determined according to EU numbering.

In some embodiments, the modified positions comprise three, four, five, six, or seven amino acid substitutions in a set of amino acid positions comprising 380, 382, 383, 424, 426, 438, and 440, wherein the positions are determined according to EU numbering. In some embodiments, the modified positions comprise three, four, five, six, seven, eight, nine, ten, or eleven amino acid substitutions in a set of amino acid positions comprising 378, 380, 382, 383, 422, 424, 426, 428, 438, 440, and 442, wherein the positions are determined according to EU numbering. In particular embodiments, the binding site includes one or more modified positions in at least one loop region. In certain embodiments, the one or more modified positions in at least one loop region are selected from the group consisting of: amino acid positions 387 and 422, wherein the positions are determined according to EU numbering. In certain embodiments, the loop region connects the two beta-strands.

In another aspect, the disclosure provides a method of introducing a non-native binding site to a TfR or to a CD98hc protein into a constant domain or non-CDR region of a variable domain of an immunoglobulin, the method comprising:
(a) generating a polynucleotide library that encodes an immunoglobulin sequence having at least three modified positions in a beta-sheet, wherein the library is randomized at codons that code for amino acids at the modified positions, wherein the modified positions are in at least two beta-strands forming the beta-sheet;
(b) expressing the library to produce a library of sequence variants;
(c) contacting the sequence variants with a TfR or CD98hc protein; and
(d) isolating sequence variants that bind to the TfR or CD98hc protein, thereby introducing a non-native binding site into a constant domain or non-CDR region of a variable domain of the immunoglobulin.

In some embodiments of this aspect, the immunoglobulin sequence comprises an Fc polypeptide.

In some embodiments, the at least two beta-strands are selected from the group consisting of: amino acid positions 239-243, 258-265, 274-278, 301-307, 319-324, 332-336, 347-351, 363-372, 378-383, 391-393, 406-412, 423-428, and 437-441, wherein the positions are determined according to EU numbering.

In some embodiments, the modified positions form a contiguous surface on the beta-sheet.

In some embodiments, the modified positions are surface accessible residues. In certain embodiments, the surface accessible residues are selected from the group consisting of: amino acid positions 347, 349, 351, 362, 364, 366, 368, 370, 378, 380, 382, 405, 407, 409, 411, 424, 426, 428, 436, 438, and 440, wherein the positions are determined according to EU numbering. In certain embodiments, the surface accessible residues are selected from the group consisting of: amino acid positions 347, 362, 378, 380, 382, 411, 424, 426, 428, 436, 438, and 440, wherein the positions are determined according to EU numbering.

In some embodiments, the modified positions comprise three, four, five, six, or seven amino acid substitutions in a set of amino acid positions comprising 380, 382, 383, 424, 426, 438, and 440, wherein the positions are determined according to EU numbering. In some embodiments, the modified positions comprise three, four, five, six, seven, eight, nine, ten, or eleven amino acid substitutions in a set of amino acid positions comprising 378, 380, 382, 383, 422, 424, 426, 428, 438, 440, and 442, wherein the positions are determined according to EU numbering.

In some embodiments, the binding site includes one or more modified positions in at least one loop region. In some embodiments, the one or more modified positions in at least one loop region are selected from the group consisting of: amino acid positions 387 and 422, wherein the positions are determined according to EU numbering.

In another aspect, the disclosure provides a method of introducing a CD98hc binding site into a polypeptide containing a beta-sheet, the method comprising:
(a) generating a polynucleotide library that encodes a polypeptide sequence having at least three modified positions in a beta-sheet, wherein the library is randomized at codons that code for amino acids at the modified positions, wherein the modified positions are in at least two beta-strands forming the beta-sheet;
(b) expressing the library to produce a library of sequence variants;
(c) contacting the sequence variants with at least a portion of the CD98hc protein; and
(d) isolating sequence variants that bind to the Cd98hc protein.

In some embodiments, the polypeptide has at least 3, 4, 5, 6, 7, 8, 9, 10, 11, 12, 13, 14, 15, 16, 17, 18, 19, or 20 modified positions in the beta-sheet. In some embodiments, the polypeptide has at least 7 modified positions in the beta-sheet. In some embodiments, the polypeptide has at least 10 modified positions in the beta-sheet.

In particular embodiments, the binding site includes one or more modified positions in at least one loop region.

In some embodiments, the binding site includes one or more beta-sheets and one or more loop regions, and at least 3, 4, 5, 6, 7, 8, 9, 10, 11, 12, 13, 14, 15, 16, 17, 18, 19, or 20 modified positions in the beta-sheet(s) and loop region(s).

In some embodiments, the polypeptide contains an immunoglobulin-like fold. In certain embodiments, the polypeptide includes an immunoglobulin (IgG) domain. In certain embodiments, the IgG domain is from an IgG, IgA, IgE, IgM, or IgD family. In certain embodiments, the IgG domain is selected is from an IgG1, IgG2, IgG3, or IgG4 molecule. In particular embodiments, the IgG domain includes a VH, CH1, CH2, CH3, VL or CL domain. In some embodiments, the randomized positions are surface accessible. In particular embodiments, the randomized positions are selected from any of those listed in Table 1B. In certain embodiments, the polypeptide includes a fibronectin or any other protein scaffold described herein.

In another aspect, the disclosure provides a polypeptide comprising a modified constant domain that specifically binds to a CD98hc protein. In some embodiments, the modified constant domain comprises a modified CH3 domain that specifically binds to the CD98hc protein. In some embodiments, the modified CH3 domain is a part of an Fc polypeptide. In particular embodiments, the CD98hc protein is a human CD98hc protein. In particular embodiments, the CD98hc protein forms a complex with LAT1 (SLC7A5), LAT2 (SLC7A8), y+LAT1 (SLC7A7), y+LAT2 (SLC7A6), Asc-1 (SLC7A10), or xCT (SLC7A11). In certain embodiments, the CD98hc protein forms a complex with LAT1 (SLC7A5).

In some embodiments, the modified constant domain (e.g., a modified CH3 domain) comprises a sequence having at least 85%, 90%, or 95% sequence identity to amino acids 111-217 of the sequence of any one of SEQ ID NOS:28-45.

In another aspect, the disclosure features a polypeptide comprising a modified constant domain (e.g., modified CH3 domain) that specifically binds to a CD98hc protein, wherein the modified constant domain comprises at least five, six, seven, eight, or nine substitutions in a set of amino acid positions consisting of 382, 384, 385, 387, 422, 424, 426, 438, 440; and wherein the positions are determined with reference to EU numbering. In another aspect, the substitutions are determined with reference to SEQ ID NO:1.

In another aspect, the disclosure features a polypeptide comprising a modified constant domain (e.g., modified CH3 domain) that specifically binds to a CD98hc protein, wherein the modified constant domain comprises at least eleven, twelve, thirteen, fourteen, or fifteen substitutions in a set of amino acid positions consisting of 380, 382, 384, 385, 386, 387, 421, 422, 424, 426, 428, 436, 438, 440 and 442; and wherein the positions are determined according to EU numbering. In another aspect, the substitutions are determined with reference to SEQ ID NO:1.

In some embodiments of this aspect, the modified constant domain (e.g., modified CH3 domain) comprises a sequence having at least 85%, 90%, or 95% sequence identity to amino acids 111-217 of the sequence of any one of SEQ ID NOS:28-43, wherein the modified constant domain comprises at least eleven, twelve, thirteen, fourteen, or fifteen substitutions in a set of amino acid positions consisting of a L at position 380, a N at position 382, a R, H, or Q at position 384, a F or Y at position 385, a V, L, I, F, Y, or E at position 386, a L at position 387, a E, Q, or A at position 421, a I, T, or P at position 422, an A at position 424, a N at position 426, a Y or W at position 428, a R or W at position 436, a F or W at position 438, a N at position 440, and an A, Q, K, R, H, or M at position 442. In some embodiments, the modified constant domain (e.g., modified CH3 domain) comprises a L at position 380, a N at position 382, a R at position 384, a F at position 385, a V at position 386, a L at position 387, an I at position 422, an A at position 424, a N at position 426, a Y at position 428, a F at position 438, a N at position 440, and an A at position 442. In particular embodiments, the modified constant domain (e.g., modified CH3 domain) comprises SEQ ID NO:28.

In some embodiments, the modified constant domain (e.g., modified CH3 domain) comprises a L at position 380, a N at position 382, a R at position 384, a F at position 385, a V at position 386, a L at position 387, an E at position 421, an I at position 422, an A at position 424, a N at position 426, a Y at position 428, a F at position 438, a N at position 440, and an A at position 442. In particular embodiments, the modified constant domain (e.g., modified CH3 domain) comprises SEQ ID NO:29.

In some embodiments, the modified constant domain (e.g., modified CH3 domain) comprises a L at position 380, a N at position 382, a Q at position 384, a Y at position 385, a E at position 386, a L at position 387, an A at position 424, a N at position 426, a Y at position 428, a F at position 438, a N at position 440, and an A at position 442. In particular embodiments, the modified constant domain (e.g., modified CH3 domain) comprises SEQ ID NO:30.

In some embodiments, the modified constant domain (e.g., modified CH3 domain) comprises a L at position 380, a N at position 382, a H at position 384, a Y at position 385, a E at position 386, a L at position 387, an A at position 424, a N at position 426, a Y at position 428, a F at position 438, a N at position 440, and an A at position 442. In particular embodiments, the modified constant domain (e.g., modified CH3 domain) comprises SEQ ID NO:31.

In some embodiments, the modified constant domain (e.g., modified CH3 domain) comprises a L at position 380, a N at position 382, a R at position 384, a F at position 385, a V at position 386, a L at position 387, an A at position 424, a N at position 426, a Y at position 428, a F at position 438, a N at position 440, and an A at position 442. In particular embodiments, the modified constant domain (e.g., modified CH3 domain) comprises SEQ ID NO:32.

In some embodiments, the modified constant domain (e.g., modified CH3 domain) comprises a L at position 380, a N at position 382, a R at position 384, a F at position 385, a V at position 386, a L at position 387, an E at position 421, an A at position 424, a N at position 426, a Y at position 428, a F at position 438, a N at position 440, and an A at position 442. In particular embodiments, the modified constant domain (e.g., modified CH3 domain) comprises SEQ ID NO:33.

In some embodiments, the modified constant domain (e.g., modified CH3 domain) comprises a L at position 380, a N at position 382, a R at position 384, a F at position 385, a V at position 386, a L at position 387, an E at position 421, an I at position 422, an A at position 424, a N at position 426, a Y at position 428, a F at position 438, and a N at position 440. In particular embodiments, the modified constant domain (e.g., modified CH3 domain) comprises SEQ ID NO:34.

In some embodiments, the modified constant domain (e.g., modified CH3 domain) comprises a L at position 380, a N at position 382, a R at position 384, a F at position 385, a V at position 386, a L at position 387, an I at position 422, an A at position 424, a N at position 426, a Y at position 428, a F at position 438, a N at position 440, and a R at position 442. In particular embodiments, the modified constant domain (e.g., modified CH3 domain) comprises SEQ ID NO:35.

In some embodiments, the modified constant domain (e.g., modified CH3 domain) comprises a L at position 380, a N at position 382, a R at position 384, a F at position 385, a V at position 386, a L at position 387, an I at position 422, an A at position 424, a N at position 426, a Y at position 428, a F at position 438, a N at position 440, and a H at position 442. In particular embodiments, the modified constant domain (e.g., modified CH3 domain) comprises SEQ ID NO:36.

In some embodiments, the modified constant domain (e.g., modified CH3 domain) comprises a L at position 380, a N at position 382, a R at position 384, a F at position 385, a V at position 386, a L at position 387, an I at position 422, an A at position 424, a N at position 426, a Y at position 428, a R at position 436, a F at position 438, a N at position 440, and a R at position 442. In particular embodiments, the modified constant domain (e.g., modified CH3 domain) comprises SEQ ID NO:37.

In some embodiments, the modified constant domain (e.g., modified CH3 domain) comprises a L at position 380, a N at position 382, a H at position 384, a Y at position 385, a E at position 386, a L at position 387, an I at position 422, an A at position 424, a N at position 426, a Y at position 428, a F at position 438, a N at position 440, and an A at position 442. In particular embodiments, the modified constant domain (e.g., modified CH3 domain) comprises SEQ ID NO:38.

In some embodiments, the modified constant domain (e.g., modified CH3 domain) comprises a L at position 380, a N at position 382, a Q at position 384, a F at position 385, a H at position 386, a L at position 387, an I at position 422, an A at position 424, a N at position 426, a Y at position 428, a F at position 438, a N at position 440, and a L at position 442. In particular embodiments, the modified constant domain (e.g., modified CH3 domain) comprises SEQ ID NO:39.

In some embodiments, the modified constant domain (e.g., modified CH3 domain) comprises a L at position 380, a N at position 382, a R at position 384, a F at position 385, a V at position 386, a L at position 387, an T at position 422, an A at position 424, a N at position 426, a Y at position 428, a F at position 438, a N at position 440, and an A at position 442. In particular embodiments, the modified constant domain (e.g., modified CH3 domain) comprises SEQ ID NO:40.

In some embodiments, the modified constant domain (e.g., modified CH3 domain) comprises a L at position 380, a N at position 382, a R at position 384, a F at position 385, a V at position 386, a L at position 387, an I at position 422, an A at position 424, a N at position 426, a Y at position 428, a F at position 438, a N at position 440, and a K at position 442. In particular embodiments, the modified constant domain (e.g., modified CH3 domain) comprises SEQ ID NO:41.

In some embodiments, the modified constant domain (e.g., modified CH3 domain) comprises a L at position 380, a N at position 382, a R at position 384, a F at position 385, a V at position 386, a L at position 387, an I at position 422, an A at position 424, a N at position 426, a Y at position 428, a W at position 436, a F at position 438, a N at position 440, and a R at position 442. In particular embodiments, the modified constant domain (e.g., modified CH3 domain) comprises SEQ ID NO:42.

In some embodiments, the modified constant domain (e.g., modified CH3 domain) comprises a L at position 380, a N at position 382, a Q at position 384, a Y at position 385, a L at position 386, a L at position 387, an E at position 421, an I at position 422, an A at position 424, a N at position 426, a Y at position 428, a F at position 438, a N at position 440, and an A at position 442. In particular embodiments, the modified constant domain (e.g., modified CH3 domain) comprises SEQ ID NO:43.

In another aspect, the disclosure features a polypeptide comprising a modified constant domain (e.g., modified CH3 domain) that specifically binds to a CD98hc protein, wherein the modified constant domain comprises:
(i) a first amino acid sequence of LX₁NX₂X₃X₄X₅L (SEQ ID NO:46), wherein X₁ is any amino acid, X₂ is R, H, or Q, X₃ is F or Y, X₄ is V, L, I, F, Y, or E, X₅ is any amino acid;
(ii) a second amino acid sequence of X₁X₂X₃AX₄X₅X₆X₇ (SEQ ID NO:47), wherein X₁ is E, N, Q, or A, X₂ is I, V, T, or P, X₃ and X₄ are any amino acid, X₅ is N or S, X₆ is any amino acid, X₇ is Y or W; and
(iii) a third amino acid sequence of X₁X₂X₃X₄NX₅X₆ (SEQ ID NO:48), wherein X₁ is Y, R, or W, X₂ is any amino acid, X₃ is F or W, X₄ and X₅ are any amino acid, and X₆ is A, Q, K, R, H, M, or S.

In some embodiments, the polypeptide binds human CD98hc with an affinity of 15 nM to 5 μM (e.g., 15 nM, 50 nM, 100 nM, 200 nM, 300 nM, 400 nM, 500 nM, 600 nM, 700 nM, 800 nM, 900 nM, 1 μM, 1.5 μM, 2 μM, 2.5 μM, 3 μM, 3.5 μM, 4 μM, 4.5 μM, or 5 μM).

In some embodiments, the polypeptide has cynomolgus monkey (cyno) cross-reactivity. In particular embodiments, the polypeptide binds to cyno CD98hc with an affinity of 80 nM to 5 μM (e.g., 80 nM, 100 nM, 200 nM, 300 nM, 400 nM, 500 nM, 600 nM, 700 nM, 800 nM, 900 nM, 1 μM, 1.5 μM, 2 μM, 2.5 μM, 3 μM, 3.5 μM, 4 μM, 4.5 μM, or 5 μM).

In another aspect, the disclosure features a polypeptide comprising a modified constant domain (e.g., modified CH3 domain) that specifically binds to a CD98hc protein wherein the modified constant domain comprises at least eight, nine, ten, eleven, twelve, or thirteen substitutions in a set of amino acid positions consisting of 380, 382, 384, 385, 386, 387, 422, 424, 426, 428, 434, 438, and 440; and wherein the substitutions are determined with reference to SEQ ID NO:1 and the positions are determined according to EU numbering. In some embodiments, the modified constant domain (e.g., modified CH3 domain) comprises a D, M, N, P, F, or H at position 380, a R, Y, F, S, W, Y, K, or N at position 382, a L, Y, A, S, or F at position 384, a F, K, D, M, I, N, Y, L, or H at position 385, a T, P, E, K, A, V, D, T, or F at position 386, a N, L, Y, R, G, S, D, or T at position 387, a I, K, R, T, F, or H at position 422, a V, W, G, L, I, P, or Y at position 424, a D, A, Q, W, L, or P at position 426, a L at position 428, a S at position 434, a I, F, N, P, or S at position 438, and a K, T, I, or F at position 440.

In another aspect, the disclosure features a polypeptide comprising a modified constant domain (e.g., modified CH3 domain) that specifically binds to a CD98hc protein wherein the modified constant domain comprises at least eight, nine, ten, eleven, twelve, thirteen, fourteen, fifteen, sixteen, seventeen, eighteen, or nineteen substitutions in a set of amino acid positions consisting of 378, 380, 382, 383, 384, 385, 386, 387, 389, 421, 422, 424, 426, 428, 434, 436, 438, 440, and 442; and wherein the substitutions are determined with reference to SEQ ID NO:1 and the positions are determined according to EU numbering. In some embodiments, the modified constant domain (e.g., modified CH3 domain) comprises a S or V at position 378, a D at position 380, a R at position 382, a T at position 383, a Y at position 384, a K at position 385, a P at position 386, a Y at position 387, a T, Y, or F at position 389, a D, E, or Q at position 421, an I at position 422, a V at position 424, a D at position 426, a L or Y at position 428, a S at position 434, a F at position 436, an I or V at position 438, a K at position 440, and a Q or M at position 442.

In another aspect, the disclosure features a polypeptide comprising a modified constant domain (e.g., modified CH3 domain) that specifically binds to a CD98hc protein wherein the modified constant domain comprises at least eight, nine, ten, eleven, twelve, thirteen, fourteen, or fifteen substitutions in a set of amino acid positions consisting of 382, 383, 384, 385, 386, 387, 389, 421, 422, 424, 426, 428, 436, 438, and 440; and wherein the substitutions are determined with reference to SEQ ID NO:1 and the positions are determined according to EU numbering. In some embodiments, the modified constant domain (e.g., modified CH3 domain) comprises a sequence having at least 85%, 90%, or 95% sequence identity to amino acids 111-217 of the sequence of any one of SEQ ID NOS:44-45, wherein the modified constant domain comprises at least eight, nine, ten, eleven, twelve, thirteen, fourteen, or fifteen substitutions in a set of amino acid positions consisting of a R at position 382, a T at position 383, a Y at position 384, a K at position 385, a P at position 386, a Y at position 387, a T at position 389, a D at position 421, an I at position 422, a V at position 424, a D at position 426, a L at position 428, a F at position 436, a I at position 438, and a K at position 440.

In some embodiments of this aspect, the modified constant domain (e.g., modified CH3 domain) comprises a R at position 382, a T at position 383, a Y at position 384, a K at position 385, a P at position 386, a Y at position 387, a T at position 389, a D at position 421, an I at position 422, a V at position 424, a D at position 426, a F at position 436, a I at position 438, and a K at position 440. In particular embodiments, the modified constant domain (e.g., modified CH3 domain) comprises SEQ ID NO:44.

In some embodiments of this aspect, the modified constant domain (e.g., modified CH3 domain) comprises a R at position 382, a T at position 383, a Y at position 384, a K at position 385, a P at position 386, a Y at position 387, a T at position 389, a D at position 421, an I at position 422, a V at position 424, a D at position 426, a L at position 428, a F at position 436, a I at position 438, and a K at position 440. In particular embodiments, the modified constant domain (e.g., modified CH3 domain) comprises SEQ ID NO:45.

In another aspect, the disclosure features a polypeptide comprising a modified constant domain (e.g., modified CH3 domain) that specifically binds to a CD98hc protein, wherein the modified constant domain comprises:
(i) a first amino acid sequence of X₁X₂YKPYX₃T (SEQ ID NO:49), wherein X₁ is E or R, X₂ is S or T, X₃ is any amino acid;
(ii) a second amino acid sequence of X₁X₂X₃VX₄DX₅X₆ (SEQ ID NO:50), wherein X₁ is N or D, X₂ is V or I, X₃, X₄, and X₅ and are any amino acid, X₆ is M or L; and
(iii) a third amino acid sequence of X₁X₂IX₃X₄ (SEQ ID NO:51), wherein X₁ is Y or F, X₂ and X₃ are any amino acid, and X₄ is S or K.

In another aspect, the disclosure features a polypeptide comprising a modified CH3 domain that specifically binds to a transferrin receptor (TfR), wherein the modified CH3 domain comprises five, six, or seven amino acid substitutions in a set of amino acid positions comprising positions 422, 424, 426, 433, 434, 438, and 440 of an Fc polypeptide (e.g., SEQ ID NO:1), wherein the modified CH3 domain does not have the combination of G at position 437, F at position 438, and D at position 440, and wherein the positions are determined according to EU numbering.

In another aspect, the disclosure provides a polypeptide comprising a modified CH3 domain that specifically binds to a transferrin receptor (TfR), wherein the modified CH3 domain comprises three, four, five, six, seven, or eight amino acid substitutions and/or one or two amino acid deletions in a set of amino acid positions comprising positions 380 and 382-389 of an Fc polypeptide (e.g., SEQ ID NO:1); and five, six, or seven amino acid substitutions in a set of amino acid positions comprising positions 422, 424, 426, 433, 434, 438, and 440 of an Fc polypeptide (e.g., SEQ ID NO:1), wherein the positions are determined according to EU numbering.

In another aspect, the disclosure provides a polypeptide comprising a modified CH3 domain that specifically binds to a transferrin receptor (TfR), wherein the modified CH3 domain comprises a sequence comprising at least one (e.g., one, two, three, four, five, six, or seven) amino acid substitution in the sequence of VFSCSVMHEALHNHYTQKS (SEQ ID NO:57), wherein the sequence of SEQ ID NO:57 is from position 422 to position 440 of an Fc polypeptide (e.g., SEQ ID NO:1), the sequence does not have the combination of G at position 437, F at position 438, and D at position 440, and the positions are determined according to EU numbering. In some embodiments of this aspect, the sequence comprises five, six, or seven amino acid substitutions in a set of amino acid positions comprising 422, 424, 426, 433, 434, 438, and 440.

In another aspect, the disclosure provides a polypeptide comprising a modified CH3 domain that specifically binds to a transferrin receptor (TfR), wherein the modified CH3 domain comprises a first sequence comprising at least one amino acid substitution (e.g., one, two, three, four, five, six, seve, or eight amino acid substitutions) and/or deletion in the sequence of AVEWESNGQPENN (SEQ ID NO:56), and a second sequence comprising at least one (e.g., one, two, three, four, five, six, or seven) amino acid substitution in the sequence of VFSCSVMHEALHNHYTQKS (SEQ ID NO:57), wherein the sequence of SEQ ID NO:56 is from position 378 to position 390 of an Fc polypeptide (e.g., SEQ ID NO:1), the sequence of SEQ ID NO:57 is from position 422 to position 440 of an Fc polypeptide (e.g., SEQ ID NO:1), and the positions are determined according to EU numbering. In some embodiments of this aspect, the modified CH3 domain comprises three, four, five, six, seven, or eight amino acid substitutions in a set of amino acid positions comprising 380 and 382-389. In some embodiments, the modified CH3 domain comprises five, six, or seven amino acid substitutions in a set of amino acid positions comprising 422, 424, 426, 433, 434, 438, and 440. In particular embodiments, the modified CH3 domain comprises one or two amino acid deletions in the sequence of SEQ ID NO:56.

In some embodiments of the above four aspects, the modified CH3 domain is part of an Fc polypeptide.

In some embodiments, the modified CH3 domain comprises F at position 382.

In some embodiments, the modified CH3 domain comprises A or a polar amino acid (e.g., Y or S) at position 383.

In some embodiments, the modified CH3 domain comprises G, N, or an acidic amino acid (e.g., D or E) at position 384.

In some embodiments, the modified CH3 domain comprises N, R, or a polar amino acid (e.g., S or T) at position 389.

In some embodiments, the modified CH3 domain comprises at least one amino acid substitution at a beta-sheet position relative to the sequence of SEQ ID NO:56.

In certain embodiments, the modified CH3 domain comprises one, two, or three amino acid substitutions at beta-sheet positions relative to the sequence of SEQ ID NO:56. In some embodiments, the beta-sheet position(s) are selected from the group consisting of: positions 380, 382, and 383, wherein the positions are determined according to EU numbering.

In certain embodiments, the modified CH3 domain comprises an amino acid substitution at beta-sheet position 380 relative to the sequence of SEQ ID NO:56. In particular embodiments, the modified CH3 domain comprises E, N, F, or Y (e.g., E) at position 380.

In some embodiments, the modified CH3 domain comprises an amino acid substitution at beta-sheet position 382 relative to the sequence of SEQ ID NO:56. In particular embodiments, the modified CH3 domain comprises F at position 382.

In some embodiments, the modified CH3 domain comprises an amino acid substitution or an amino acid deletion at beta-sheet position 383 relative to the sequence of SEQ ID NO:56. In particular embodiments, the modified CH3 domain comprises Y or A (e.g., Y) at position 383.

In some embodiments, the modified CH3 domain comprises at least one amino acid substitution at a beta-sheet position relative to the sequence of SEQ ID NO:57. In some embodiments, the modified CH3 domain comprises at one, two, three, or four amino acid substitutions at beta-sheet positions relative to the sequence of SEQ ID NO:57. In particular embodiments, the beta-sheet position(s) are selected from the group consisting of: positions 424, 426, 438, and 440, according to EU numbering.

In some embodiments, the modified CH3 domain comprises an amino acid substitution at beta-sheet position 424 relative to the sequence of SEQ ID NO:57. In particular embodiments, the modified CH3 domain comprises A at position 424.

In some embodiments, the modified CH3 domain comprises an amino acid substitution at beta-sheet position 426 relative to the sequence of SEQ ID NO:57. In particular embodiments, the modified CH3 domain comprises E at position 426.

In some embodiments, the modified CH3 domain comprises an amino acid substitution at beta-sheet position 438 relative to the sequence of SEQ ID NO:57. In particular embodiments, the modified CH3 domain comprises Y at position 438.

In some embodiments, the modified CH3 domain comprises an amino acid substitution at beta-sheet position 440 relative to the sequence of SEQ ID NO:57. In particular embodiments, the modified CH3 domain comprises L at position 440.

In certain embodiments, the modified CH3 domain comprises H or E (e.g., H) at position 433.

In some embodiments, the modified CH3 domain comprises N or G (e.g., N) at position 434.

In some embodiments, the modified CH3 domain comprises at least one position selected from the following: E, N, F, or Y at position 380, F at position 382, Y, S, A, or an amino acid deletion at position 383, G, D, E, or N at position 384, D, G, N, or A at position 385, Q, S, G, A, or N at position 386, K, I, R, or G at position 387, E, L, D, or Q at position 388, and N, T, S, or R at position 389. In particular embodiments, the modified CH3 domain comprises five, six, seven, or eight positions selected from the following: F at position 382, Y or S at position 383, G, D, or E at position 384, D, G, N, or A at position 385, Q, S, or A at position 386, K at position 387, E or L at position 388, N, T, or S at position 389.

In some embodiments, the modified CH3 domain comprises at least one position selected from the following: L at position 422, A at position 424, E at position 426, H or E at position 433, N or G at position 434, Y at position 438, and L at position 440. In particular embodiments, the modified CH3 domain comprises five positions selected from the following: L at position 422, A at position 424, E at position 426, Y at position 438, and L at position 440.

In another aspect, the disclosure provides a polypeptide comprising a modified CH3 domain that specifically binds to a transferrin receptor (TfR), wherein the modified CH3 domain comprises: (i) a sequence of AVX₁WFX₂X₃X₄X₅X₆X₇X₈N (SEQ ID NO:65), wherein X₁ is E, N, F, or Y; X₂ is Y, S, A, or absent; X₃ is G, D, E, or N; X₄ is D, G, N, or A; X₅ is Q, S, G, A, or N; X₆ is K, I, R, or G; X₇ is E, L, D, or Q; and X₈ is N, T, S, or R; and (ii) a sequence of LFACEVMHEALX₁X₂HYTYKL (SEQ ID NO:67), wherein X₁ is H or E; and X₂ is N or G.

In some embodiments of the above five aspects, the modified CH3 domain comprises a sequence of AVEWFYDDSKLTN (SEQ ID NO:58), AVEWFYGNAKETN (SEQ ID NO:59), AVEWFYEAQKLNN (SEQ ID NO:60), AVEWFSEGSKETN (SEQ ID NO:61), AVEWFSGAQKESN (SEQ ID NO:62), or AVEWFSGAQKLTN (SEQ ID NO:63). In some embodiments, the modified CH3 domain comprises the sequence of LFACEVMHEALHNHYTYKL (SEQ ID NO:64).

In certain embodiments, the modified CH3 domain comprises the sequence of AVEWFYDDSKLTN (SEQ ID NO:58) and the sequence of LFACEVMHEALHNHYTYKL (SEQ ID NO:64). In certain embodiments, the modified CH3 domain comprises the sequence of AVEWFYGNAKETN (SEQ ID NO:59) and the sequence of LFACEVMHEALHNHYTYKL (SEQ ID NO:64). In certain embodiments, the modified CH3 domain comprises the sequence of AVEWFYEAQKLNN (SEQ ID NO:60) and the sequence of LFACEVMHEALHNHYTYKL (SEQ ID NO:64). In certain embodiments, the modified CH3 domain comprises the sequence of AVEWFSEGSKETN (SEQ ID NO:61) and the sequence of LFACEVMHEALHNHYTYKL (SEQ ID NO:64). In certain embodiments, the modified CH3 domain comprises the sequence of AVEWFSGAQKESN (SEQ ID NO:62) and the sequence of LFACEVMHIIEALHNHYTYKL (SEQ ID NO:64). In certain embodiments, the modified CH3 domain comprises the sequence of AVEWFSGAQKLTN (SEQ ID NO:63) and the sequence of LFACEVMHEALHNHYTYKL (SEQ ID NO:64).

In further embodiments of the above five aspects, the modified CH3 domain further comprises one, two, three, four, or five amino acid substitutions at positions comprising 419-421, 442, and 443, wherein the positions are determined according to EU numbering. In some embodiments, the modified CH3 domain comprises Q or P at position 419, G or R at position 420, N or G at position 421, S or G at position 442, and/or L or E at position 443.

In some embodiments, the modified CH3 domain comprises a sequence having at least 85% identity, at least 90% identity, or at least 95% identity to amino acids 111-217 of any one of SEQ ID NOS:72-77. In some embodiments, the modified CH3 domain comprises amino acids 111-217 of any one of SEQ ID NOS:72-77.

In some embodiments, the polypeptide comprises a sequence having at least 85% identity, at least 90% identity, or at least 95% identity to a sequence of any one of SEQ ID NOS:72-77. In some embodiments, the polypeptide comprises a sequence of any one of SEQ ID NOS:72-77.

In another aspect, the disclosure provides a polypeptide comprising a modified CH3 domain that specifically binds to a transferrin receptor (TfR), wherein the modified CH3 domain comprises: F at position 382, Y at position 383, D at position 384, D at position 385, S at position 386, K at position 387, L at position 388, T at position 389, P at position 419, R at position 420, G at position 421, L at position 422, A at position 424, E at position 426, Y at position 438, L at position 440, G at position 442, and E at position 443, wherein the positions are determined according to EU numbering.

In another aspect, the disclosure provides a polypeptide comprising a modified CH3 domain that specifically binds to a transferrin receptor (TfR), wherein the modified CH3 domain comprises: F at position 382, Y at position 383, G at position 384, N at position 385, A at position 386, K at position 387, T at position 389, L at position 422, A at position 424, E at position 426, Y at position 438, L at position 440, wherein the positions are determined according to EU numbering.

In another aspect, the disclosure provides a polypeptide comprising a modified CH3 domain that specifically binds to a transferrin receptor (TfR), wherein the modified CH3 domain comprises: F at position 382, Y at position 383, E at position 384, A at position 385, K at position 387, L at position 388, L at position 422, A at position 424, E at position 426, Y at position 438, L at position 440, wherein the positions are determined according to EU numbering.

In another aspect, the disclosure provides a polypeptide comprising a modified CH3 domain that specifically binds to a transferrin receptor (TfR), wherein the modified CH3 domain comprises: F at position 382, E at position 384, S at position 386, K at position 387, T at position 389, L at position 422, A at position 424, E at position 426, Y at position 438, L at position 440, wherein the positions are determined according to EU numbering.

In another aspect, the disclosure provides a polypeptide comprising a modified CH3 domain that specifically binds to a transferrin receptor (TfR), wherein the modified CH3 domain comprises: F at position 382, G at position 384, A at position 385, K at position 387, S at position 389, L at position 422, A at position 424, E at position 426, Y at position 438, L at position 440, wherein the positions are determined according to EU numbering.

In another aspect, the disclosure provides a polypeptide comprising a modified CH3 domain that specifically binds to a transferrin receptor (TfR), wherein the modified CH3 domain comprises: F at position 382, G at position 384, A at position 385, K at position 387, L at position 388, T at position 389, L at position 422, A at position 424, E at position 426, Y at position 438, L at position 440, wherein the positions are determined according to EU numbering.

In another aspect, the disclosure a polypeptide comprising a sequence of any one of SEQ ID NOS:72, 78, 84, 90, 96, 102, 108, 114, and 120.

In another aspect, the disclosure a polypeptide comprising a sequence of any one of SEQ ID NOS:73, 79, 85, 91, 97, 103, 109, 115, and 121.

In another aspect, the disclosure a polypeptide comprising a sequence of any one of SEQ ID NOS:74, 80, 86, 92, 98, 104, 110, 116, and 122.

In another aspect, the disclosure a polypeptide comprising a sequence of any one of SEQ ID NOS:75, 81, 87, 93, 99, 105, 111, 117, and 123.

In another aspect, the disclosure a polypeptide comprising a sequence of any one of SEQ ID NOS:76, 82, 88, 94, 100, 106, 112, 118, and 124.

In another aspect, the disclosure a polypeptide comprising a sequence of any one of SEQ ID NOS:77, 83, 89, 95, 101, 107, 113, 119, and 125.

In another aspect, the disclosure provides an Fc polypeptide that specifically binds to TfR, comprising a modified CH3 domain, wherein the modified CH3 domain comprises a sequence at least 85% (e.g., at least 90%, 91%, 93%, 95%, 97%, 98%, or 99%) identical to amino acids 111-217 of the sequence of SEQ ID NO:137, wherein the modified CH3 domain comprises Ala, Asp, His, Tyr, or Phe at position 378; Ala, Asp, Phe, Leu, Gln, Glu, or Lys at position 380; Gly at position 382; Leu, Ala, or Glu at position 384; Val at position 385; Gln or Ala at position 386; Val, Ile, Phe, or Leu at position 422; Ser, Ala, or Pro at position 424; Thr or Ile at position 426; Ile or Tyr at position 438; and Gly, Ser, Thr, or Val at position 440. In some embodiments, the modified CH3 domain has Met or Leu at position 428.

In another aspect, the disclosure provides an Fc polypeptide that specifically binds to TfR, comprising a modified CH3 domain, wherein the modified CH3 domain comprises a sequence at least 85% (e.g., at least 90%, 91%, 93%, 95%, 97%, 98%, or 99%) identical to amino acids 111-217 of the sequence of SEQ ID NO:137, wherein the modified CH3 domain comprises any set of substitutions provided for any of the clones in Tables 32B-1, 32C, 32D, 32E, 32F, 323G, 32H, 32H-1, 32J, and 32K, or comprises the possible amino acids set forth in Table 32I.

In some embodiments of the above two aspects, the modified CH3 domain comprises Ala or His at position 378; Asp or Glu at position 380; Gly at position 382; Leu at position 384; Val at position 385; Gln or Ala at position 386; Ile or Val at position 422; Ala or Pro at position 424; Thr or Ile at position 426; Ile at position 438; and Gly or Thr at position 440, according to EU numbering. The modified CH3 domain may also include Met or Leu at position 428.

In some embodiments, the modified CH3 domain comprises His at position 378; Glu at position 380; Gly at position 382; Leu at position 384; Val at position 385; Gln at position 386; Ile at position 422; Pro at position 424; Ile at position 426; Ile at position 438; and Thr at position 440, according to EU numbering. The modified CH3 domain may also include Met or Leu at position 428.

In some embodiments, the modified CH3 domain comprises His at position 378; Glu at position 380; Gly at position 382; Leu at position 384; Val at position 385; Gln at position 386; Ile at position 422; Pro at position 424; Ile at position 426; Leu at position 428; Ile at position 438; and Thr at position 440, according to EU numbering.

In another aspect, the disclosure provides an Fc polypeptide that specifically binds to TfR, comprising a modified CH3 domain, wherein the modified CH3 domain comprises a sequence at least 85% (e.g., at least 90%, 91%, 93%, 95%, 97%, 98%, or 99%) identical to amino acids 111-217 of the sequence of SEQ ID NO:137, wherein the modified CH3 domain comprises His at position 378; Glu at position 380; Gly at position 382; Leu at position 384; Val at position 385; Gln at position 386; Ile at position 422; Pro at position 424; Ile at position 426; Ile at position 438; and Thr at position 440, according to EU numbering. The modified CH3 domain may also include Met or Leu at position 428.

In another aspect, the disclosure provides an Fc polypeptide that specifically binds to TfR, comprising a modified CH3 domain, wherein the modified CH3 domain comprises a sequence at least 85% (e.g., at least 90%, 91%, 93%, 95%, 97%, 98%, or 99%) identical to amino acids 111-217 of the sequence of SEQ ID NO:138, wherein the modified CH3 domain comprises His at position 378; Glu at position 380; Gly at position 382; Leu at position 384; Val at position 385; Gln at position 386; Ile at position 422; Pro at position 424; Ile at position 426; Leu at position 428; Ile at position 438; and Thr at position 440, according to EU numbering. In some embodiments of the disclosure provided herein, the modified constant domain (e.g., modified CH3 domain) further comprises at least one modification that promotes heterodimerization. In particular embodiments, the modified constant domain (e.g., modified CH3 domain) further comprises a T366W substitution, according to EU numbering. In particular embodiments, the modified constant domain (e.g., modified CH3 domain) further comprises T366S, L368A, and Y407V substitutions, according to EU numbering.

In some embodiments of the disclosure provided herein, the modified constant domain (e.g., modified CH3 domain) further comprises a CH2 domain (e.g., a modified CH2 domain). In some embodiments, the modified CH2 and CH3 domains form an Fc polypeptide. In particular embodiments, the modified CH2 domain comprises modifications that reduce effector function. In particular embodiments, the CH2 domain comprises Ala at position 234 and Ala at position 235, according to EU numbering. In particular embodiments, the CH2 domain comprises Ala at position 234, Ala at position 235, and Gly at position 329, according to EU numbering. In particular embodiments, the CH2 domain comprises Ala at position 234, Ala at position 235, and Ser at position 329, according to EU numbering.

In some embodiments, the CH2 domain is a human IgG1, IgG2, IgG3, or IgG4 CH2 domain.

In some embodiments of any aspects described herein, the polypeptide is part of a dimer. In some embodiments, the dimer is an Fc dimer. In some embodiments, the polypeptide is further joined to a Fab.

In some embodiments of any aspects described herein, the polypeptide is a first polypeptide of a dimer such that the dimer is monovalent for CD98hc binding. In other embodiments, the polypeptide is a first polypeptide of a dimer such that the dimer is bivalent for CD98hc binding.

In some embodiments of any aspects described herein, the polypeptide is a first polypeptide of a dimer such that the dimer is monovalent for TfR binding. In other embodiments, the polypeptide is a first polypeptide of a dimer such that the dimer is bivalent for TfR binding.

In some embodiments of any aspects described herein, the C-terminal lysine of polypeptide is removed.

In another aspect, the disclosure provides a polynucleotide comprising a nucleic acid sequence encoding a polypeptide described herein.

In another aspect, the disclosure provides a vector comprising the polynucleotide comprising a nucleic acid sequence encoding a polypeptide described herein.

In another aspect, the disclosure provides a host cell comprising the polynucleotide comprising a nucleic acid sequence encoding a polypeptide described herein.

In another aspect, the disclosure provides a method for producing a polypeptide comprising a modified constant domain (e.g., modified CH3 domain), comprising culturing a host cell under conditions in which the polypeptide encoded by the polynucleotide described herein is expressed.

In another aspect, the disclosure provides a pharmaceutical composition comprising a polypeptide described herein and a pharmaceutically acceptable carrier.

In another aspect, the disclosure provides a method of transcytosis of a therapeutic agent across an endothelium. In some embodiments, the method comprises contacting the endothelium with a composition comprising a polypeptide dimer capable of binding CD98hc (e.g., a polypeptide dimer described herein) fused to a therapeutic agent. In some embodiments, the method comprises contacting the endothelium with a composition comprising a polypeptide dimer capable of binding TfR (e.g., a polypeptide dimer described herein) fused to a therapeutic agent. In some embodiments, the endothelium is the BBB.

In another aspect, the disclosure provides a method for engineering a polypeptide comprising a modified CH3 domain to specifically bind to a CD98hc protein, the method comprising:
(a) modifying a polynucleotide that encodes the modified CH3 domain to comprise: (i) a first sequence comprising at least one substitution relative to the sequence of EWESNGQP (SEQ ID NO:52), (ii) a second sequence comprising at least one substitution relative to the sequence ofNVFSCSVM (SEQ ID NO:53), and (iii) a third sequence comprising at least one substitution relative to the sequence of YTQKSLS (SEQ ID NO:54);
(b) expressing and recovering a polypeptide comprising the modified CH3 domain; and
(c) determining whether the polypeptide binds to a CD98hc protein,
   wherein the sequence of SEQ ID NO:52 is from position 380 to position 387 of an Fc polypeptide (e.g., SEQ ID NO:1), the sequence of SEQ ID NO:53 is from position 421 to position 428 of an Fc polypeptide (e.g., SEQ ID NO:1), the sequence of SEQ ID NO:54 is from position 436 to position 442 of an Fc polypeptide (e.g., SEQ ID NO:1) and the positions are determined according to EU numbering.

In another aspect, the disclosure provides a method for engineering a polypeptide comprising a modified CH3 domain to specifically bind to a TfR protein, the method comprising:
(a) modifying a polynucleotide that encodes the modified CH3 domain to comprise: (i) a first sequence comprising at least one amino acid substitution and/or deletion relative to the sequence of AVEWESNGQPENN (SEQ ID NO:56), and (ii) a second sequence comprising at least one amino acid substitution in the sequence of VFSCSVIHEALHNHYTQKS (SEQ ID NO:57);
(b) expressing and recovering the polypeptide comprising the modified CH3 domain; and
(c) determining whether the polypeptide binds to the TfR protein,
   wherein the sequence of SEQ ID NO:56 is from position 378 to position 390 of an Fc polypeptide (e.g., SEQ ID NO:1), and the sequence of SEQ ID NO:57 is from position 422 to position 440 of an Fc polypeptide (e.g., SEQ ID NO:1), and the positions are determined according to EU numbering.

In some embodiments of this aspect, the steps of expressing the polypeptide comprising the modified CH3 domain and determining whether the modified CH3 domain binds to CD98hc or TfR are performed using a display system. In particular embodiments, the display system is a cell surface display system, a viral display system, an mRNA display system, a polysomal display system, or a ribosomal display system.

In another aspect, the disclosure provides a method of delivering a therapeutic agent across the BBB into the brain parenchyma, the method comprising contacting the BBB with a composition comprising a polypeptide dimer described herein fused to a therapeutic agent.

In another aspect, the disclosure provides a method of delivering a therapeutic agent across the BBB to target an extracellular target, the method comprising contacting the BBB with a composition comprising a polypeptide dimer described herein fused to a therapeutic agent.

In some embodiments of the above two aspects, one polypeptide in the polypeptide dimer comprises at least eleven, twelve, thirteen, fourteen, or fifteen substitutions in a set of amino acid positions consisting of 378, 380, 382, 383, 384, 385, 386, 387, 389, 391, 421, 422, 424, 426, 428, 434, 436, 438, 440, 441, and 442, according to EU numbering.

In some embodiments of the above two aspects, one polypeptide in the polypeptide dimer comprises at least eight, nine, eleven, twelve, thirteen, fourteen, fifteen, sixteen, seventeen, eighteen, or nineteen substitutions in a set of amino acid positions consisting of 378, 380, 382, 383, 384, 385, 386, 387, 389, 421, 422, 424, 426, 428, 434, 436, 438, 440, and 442, according to EU numbering.

In some embodiments, both polypeptides in the polypeptide dimer do not have the substitutions L234A, L235A, and P329G.

In another aspect, the disclosure provides a method of delivery across the BBB to a biological target in the brain, the method comprising: (a) a CD98hc-binding polypeptide described herein, and (b) a means for binding the biological target in the brain.

In some embodiments, the biological target is a cell surface target in the brain, such as on a microglial cell, an astrocyte, an oligodendrocyte, a neuron, and a cancer cell. In some embodiments, the cell surface target is selected from the group consisting of TREM2, PILRA, CD33, CR1, ABCA1, ABCA7, MS4A4A, MS4A6A, MS4A4E, HLA-DR5, HLA-DR1, IL1RAP, TREML2, IL-34, SORL1, ADAM17, and Siglec11.

In some embodiments, the biological target is a cell surface target on a hematological cancer cell. In certain embodiments, the cell surface target is selected from the group consisting of B7H3, BCMA, CD125, CD166, CD19, CD20, CD205, CD22, CD25, CD30, CD37, CD39, CD73, and CD79b.

In some embodiments, the target is on a tumor cell. In certain embodiments, the target is selected from the group consisting of ALK, AXL, CD25, CD44v6, CD46, CD56 (NCAM), CDH6 (cadherin 6), CEACAM 5 (CD66E), EGFR, EGFR viii, ETBR, FGFR (1-4), Folate Receptor alpha, GAL-3BP (galectin binding protein), GD2, GD3, GloboH (globohexasylceramide), gp100, gpNMB, HER2, HER3, HER4, IGFR1, KIT, LIV1A, LRRC15 (leucine rich repeat containing 15), MET, NaPi2B, PDL1, PMEL17, PRAME, PSMA, PTK7 (CCK4; colon carcinoma kinase), RON, ROR1, TF (tissue factor), and TROP2.

In some embodiments, the target may include alpha-synuclein or derivatives or fragments thereof, amyloid-beta peptide or derivatives of fragments thereof, Tau or derivatives or fragments thereof, pTau, huntingtin, transthyretin, or TAR DNA-binding protein 43 (TDP-43) or derivatives or fragments thereof.

In another aspect, the disclosure provides a method of targeting an extracellular target in the brain with a CD98hc-binding polypeptide, the method comprising administering the CD98hc-binding polypeptide to a patient, wherein the polypeptide is transported across the BBB and into the parenchyma without being transcytosed into a cell within the brain. In some embodiments, the extracellular target is on or near an astrocyte, microglia, oligodendrocyte, or a cancer cell. In certain embodiments, the extracellular target is an antigen in the brain. In certain embodiments, the antigen is a plaque, tangle, or other non-cellular target. In some embodiments, the extracellular target is a non-neuronal target. In certain embodiments, the method comprises delivering a therapeutic agent to the extracellular target.

In another aspect, the disclosure provides a method of delivering a therapeutic agent across the BBB to astrocyte cells, the method comprising contacting the BBB with a composition comprising a polypeptide dimer described herein fused to a therapeutic agent. In some embodiments, both polypeptides in the polypeptide dimer comprises at least eleven, twelve, thirteen, fourteen, or fifteen substitutions in a set of amino acid positions consisting of 378, 380, 382, 383, 384, 385, 386, 387, 389, 391, 421, 422, 424, 426, 428, 434, 436, 438, 440, 441, and 442, according to EU numbering.

In another aspect, the disclosure provides a method of delivering a therapeutic agent to a peripheral CD98hc expressing organ comprising administering to a subject a composition comprising a polypeptide dimer described herein fused to a therapeutic agent. In certain embodiments, the peripheral CD98hc expressing organ is kidney, testes, bone marrow, spleen, or pancreas.

In another aspect, the disclosure provides a CD98hc binding polypeptide, wherein, when bound to human CD98hc, the polypeptide binds to at least 7, 8, 9, 10, 11, 12, 13 or 14 of the residues selected from positions of the group consisting of: 477, 478, 479, 480, 481, 482, 483, 486, 499, 497, 498, 500, 501, and 502 of SEQ ID NO: 134. In certain embodiments, when bound to human CD98hc, the polypeptide binds to positions 477, 478, 479, 480, 481, 482, 483, 486, 499, 497, 498, 500, 501, and 502 of SEQ ID NO: 134. In certain embodiments, when bound to human CD98hc, the polypeptide binds additionally to at least 1 additional residue selected from positions of the group consisting of: 229, 231, 232, 236, 235, 488, 495, and 496 of SEQ ID NO: 134. In certain embodiments, when bound to human CD98hc, the polypeptide binds additionally to at least 1 additional residue selected from positions of the group consisting of: 312, 315, 348, 381, 439, 444, 443, 485, 484, 476, 475, and 442 of SEQ ID NO: 134.

In another aspect, the disclosure provides a CD98hc binding polypeptide, wherein, when bound to human CD98hc, the polypeptide binds to at least 11, 12, 13, 14, 15, 16, 17, 18, 19, 20, 21 or 22 of the residues selected from positions of the group consisting of: 229, 231, 232, 236, 235, 486, 488, 495, 496, 498, 500, 499, 497, 482, 481, 483, 477, 480, 501, 502, 478, and 479 of SEQ ID NO:134.

In another aspect, the disclosure provides a CD98hc binding polypeptide, wherein, when bound to human CD98hc, the polypeptide binds to at least 13, 14, 15, 16, 17, 18, 19, 20, 21, 22, 23, 24, 25, or 26 of the residues selected from positions of the group consisting of: 312, 315, 348, 381, 439, 444, 443, 485, 484, 477, 483, 481, 480, 478, 476, 502, 499, 501, 500, 498, 497, 486, 479, 482, 475, and 442 of SEQ ID NO: 134. In certain embodiments, the polypeptide is an antibody or fragment thereof, a VHH domain, or a polypeptide comprising a modified constant domain that specifically binds to a CD98hc protein.

In another aspect, the disclosure provides a method of increasing brain exposure to a therapeutic agent in a subject relative to a reference molecule, the method comprising administering to the subject a monovalent molecule that binds to CD98hc with a binding affinity from about 20 nM to about 550 nM, wherein the molecule is linked to the therapeutic agent, and wherein the reference molecule comprises the therapeutic agent but not a CD98hc binding moiety.

In another aspect, the disclosure provides a method of increasing brain exposure to a therapeutic agent in a subject relative to reference molecule, the method comprising administering to the subject a bivalent molecule that binds to CD98hc with a binding affinity from about 275 nM to about 2100 nM, wherein the molecule is linked to the therapeutic agent, and wherein the reference molecule comprises the therapeutic agent but not a CD98hc binding moiety.

In another aspect, the disclosure provides a composition for delivery across the BBB to a biological target in the brain, the composition comprising: (a) a CD98hc-binding polypeptide described herein, and (b) a means for binding the biological target in the brain.

In some embodiments, the biological target is a cell surface target in the brain, such as on a microglial cell, an astrocyte, an oligodendrocyte, a neuron, and a cancer cell. In some embodiments, the cell surface target is selected from the group consisting of TREM2, PILRA, CD33, CR1, ABCA1, ABCA7, MS4A4A, MS4A6A, MS4A4E, HLA-DR5, HLA-DR1, IL1RAP, TREML2, IL-34, SORL1, ADAM17, and Siglec11.

In some embodiments, the biological target is a cell surface target on a hematological cancer cell. In certain embodiments, the cell surface target is selected from the group consisting of B7H3, BCMA, CD125, CD166, CD19, CD20, CD205, CD22, CD25, CD30, CD37, CD39, CD73, and CD79b.

In some embodiments, the target is on a tumor cell. In certain embodiments, the target is selected from the group consisting of ALK, AXL, CD25, CD44v6, CD46, CD56 (NCAM), CDH6 (cadherin 6), CEACAM 5 (CD66E), EGFR, EGFR viii, ETBR, FGFR (1-4), Folate Receptor alpha, GAL-3BP (galectin binding protein), GD2, GD3, GloboH (globohexasylceramide), gp100, gpNMB, HER2, HER3, HER4, IGFR1, KIT, LIV1A, LRRC15 (leucine rich repeat containing 15), MET, NaPi2B, PDL1, PMEL17, PRAME, PSMA, PTK7 (CCK4; colon carcinoma kinase), RON, ROR1, TF (tissue factor), and TROP2.

In some embodiments, the target may include alpha-synuclein or derivatives or fragments thereof, amyloid-beta peptide or derivatives of fragments thereof, Tau or derivatives or fragments thereof, pTau, huntingtin, transthyretin, or TAR DNA-binding protein 43 (TDP-43) or derivatives or fragments thereof.

In another aspect, the disclosure provides a method for delivery across the BBB to a biological target in the brain of a subject, the method comprising:
(a) providing a composition comprising (i) a CD98hc-binding polypeptide described herein, and (ii) a means for binding the biological target; and
(b) peripherally administering the composition of step (a) to the subject.

In another aspect, the disclosure provides a method for binding a biological target in the brain of a subject, the method comprising:
(a) providing a composition comprising (i) a CD98hc-binding polypeptide described herein, and (ii) a means for binding the biological target;
(b) peripherally administering the composition of step (a) to the subject;
   wherein the composition binds the biological target in the brain of the subject.

Unless otherwise indicated or apparent from the context, all numbering for positions throughout this document in an Fc, CH2, or CH3 polypeptide (e.g., “position x”) is based on the EU numbering system.

### BRIEF DESCRIPTION OF THE DRAWINGS

FIG. 1 illustrates plasma pharmacokinetics of LLB2 and LLB1 CD98hc-binding molecules in C57/B6 (WT) mice.

FIG. 2 illustrates plasma pharmacokinetics of additional LLB2 and LLB1 CD98hc-binding molecules in C57/B6 (WT) mice.

FIG. 3 illustrates plasma pharmacokinetics of affinity matured LLB2 CD98hc-binding molecules in C57/B6 (WT) mice.

FIG. 4 illustrates plasma pharmacokinetics of de-affinity matured LLB2 CD98hc-binding molecules in C57/B6 (WT) mice.

FIGS. 5A-5C illustrate brain uptake of LLB2 and LLB1 CD98hc-binding molecules in CD98hc^{mu/hu} KI mice. (A) huIgG in plasma 48 hr post dose. (B) huIgG in whole brain lysate. (C) ratio of huIgG in brain to plasma.

FIG. 6 illustrates capillary depletion demonstrating that CD98hc-binding molecules cross the BBB into the brain parenchyma of CD98hc^{mu/hu} KI mice.

FIG. 7 illustrates CNS biodistribution of LLB2 and LLB1 variants in CD98hc^{mu/hu} KI mice.

FIG. 8 illustrates cell specific biodistribution of CD98hc-binding molecules:IBA1 (microglia) by immunohistochemistry in CD98hc^{m}u/hu KI mice.

FIG. 9 illustrates cell specific biodistribution of CD98hc-binding molecules:AQPN4 (astrocytes) by immunohistochemistry in CD98hc^{m}u/hu KI mice.

FIGS. 10A and 10B illustrate brain uptake of additional LLB2 and LLB1 variants in CD98hc^{mu/hu} KI mice. (A) huIgG in plasma 48 hr post dose. (B) huIgG in whole brain lysate.

FIGS. 11A-11F illustrate peripheral tissue localization of LLB2 and LLB1 variants in CD98hc^{mu/hu} KI mice.

FIGS. 12A and 12B illustrate brain uptake timecourse of monovalent and bivalent LLB2 variants in CD98hc^{mu/hu} KI mice. (A) huIgG PK in plasma out to 10 days post dose. (B) huIgG PK in whole brain lysate.

FIG. 13 illustrates capillary depletion demonstrating that monovalent and bivalent LLB2 variants cross the BBB into the brain parenchyma of CD98hc^{mu/hu} KI mice.

FIGS. 14A-14H illustrate huIgG pharmacokinetics (PK) in peripheral tissues of monovalent and bivalent LLB2 variants in CD98hc^{mu/hu} KI mice.

FIG. 15 illustrates biodistribution timecouse of monovalent and bivalent LLB2-10-8 by immunohistochemistry for huIgG.

FIG. 16 illustrates biodistribution timecouse of monovalent and bivalent LLB2-10-8 by immunohistochemistry for huIgG and Iba1 (microglia).

FIGS. 17A and 17B illustrate (A) plasma and (B) brain exposure after repeat dosing of monovalent LLB2 variants.

FIG. 18 illustrates biodistribution timecouse after repeat dosing of monovalent LLB2-10⁻⁸ variants.

FIGS. 19A and 19B illustrate the yeast display library surface, modeled onto a wild-type IgG1 Fc backbone (PDB 1hzh).

FIGS. 20A-20C illustrate the concentrations of clones and controls in plasma (FIG. 20A) and whole brain (FIG. 20B) of chimeric huTfR^{apical} knock-in mice at 24 hours after administering the mice with 50 mg/kg dose of clone or control. FIG. 20C shows the reduction of Aβ40 levels in the brain of the mice.

FIGS. 21A-21G illustrate the plasma PK of clones and controls with a 10 mg/kg dose in wild-type mice (FIG. 21A). The brain PK (FIG. 21), brain PD (FIG. 21C), and plasma PK (FIG. 21D) of monovalent clone 6.5.11.5.42.2, bivalent clone 6.5.11.5.42.2, and controls with a 50 mg/kg dose in chimeric huTfR^{apical} knock-in mice. FIGS. 3E and 3F show safety data showing percent of Ter119⁺erythrocytes (FIG. 21E) or CD71⁺bone marrow reticulocytes (FIG. 21F) in the total plasma cell population for anti-BACE1 control or bivalent clone 6.5.11.5.42.2. FIG. 21G shows the level of whole brain TfR compared to loading control GAPDH 24 hours after treatment.

FIG. 22 illustrates size-exclusion chromatography (SEC) analysis of Clone 6.5.11.5.42.2 under low pH and control conditions.

FIGS. 23A-23C illustrate the structure of clone 6.5.11.5.42 with human TfR circularly permuted apical domain, with the library residues in sticks (FIG. 23A). Structure of clone 6.5.11.5.42 with TfR apical domain and modeled full-length human TfR domain (FIG. 23B). Zoom of FIG. 23B that shows the clash between human TfR domains (FIG. 23C).

FIGS. 24A and 24B illustrate the concentrations of monovalent and bivalent clones 42.2.1.2, monovalent and bivalent clones 6.5.11.5.42.2, and control in whole brain (FIG. 24A) and plasma (FIG. 24B) of chimeric huTfR^{apical} knock-in mice at 24 hours after administering the mice with 50 mg/kg dose of clone or control.

FIG. 25 shows safety data showing recticulocyte level for monovalent and bivalent clones 42.2.1.2, monovalent and bivalent clones 6.5.11.5.42.2, and control.

FIGS. 26A-26D illustrate the plasma PK (FIGS. 26A and 26B) and brain PK (FIGS. 26C and 26D) of monovalent and bivalent clones 42.8.17, 42.8.15, 42.8.80, 42.8.196, 42.2.3-1H, and 42.2.19 and control with a 50 mg/kg dose in chimeric huTfR^{apical} knock-in mice.

FIGS. 27A and 27B illustrate (A) plasma and (B) brain exposure after repeat dosing of bivalent LLB2 variants in CD98hc^{mu/hu} KI mice.

FIGS. 28A and 28B show the crystal structure of bivalent CD98hc binding molecule with CD98hc: (A) LLB2-10⁻⁶ dimer (B) LLB1-3-16 dimer.

FIGS. 29A and 29B show co-complex of CD98hc binding molecule crystal structure: with CD98hc and modelled with two FcRn-B2M: (A) LLB2-10⁻⁶ dimer (B) LLB1-3-16 dimer.

FIGS. 30A-30C show orientation of a bivalent CD98hc binding molecule crystal structure with respect to the CD98hc-LAT1 complex modelled: (A) bivalent LLB2-10⁻⁶ dimer, (B) bivalent LLB1-3-16 dimer, and (C) monovalent LLB2-10⁻⁶ dimer.

FIGS. 31A-31F show plasma and brain PK and capillary depletion results in CD98hc^{mu/hu} KI mice post-dose with monovalent LLB2 variants: (A) plasma PK, (B) brain PK, (C) parenchymal fraction, (D) vasculature fraction, (E) cell associated fraction, and (F) non-cell associated fraction.

FIGS. 32A-32F show plasma and brain PK and capillary depletion results in CD98hc^{mu/hu} KI mice post-dose with bivalent LLB2 variants: (A) plasma PK, (B) brain PK, (C) parenchymal fraction, (D) vasculature fraction, (E) cell associated fraction, and (F) non-cell associated fraction.

FIGS. 33A and 33B show huIgG PK in (A) plasma and (B) whole brain lysate out to 21 days post dose in affinity matched LLB1 and LLB2 variants.

FIGS. 34A-34F show plasma and brain PK and capillary depletion results in CD98hc^{mu/hu} KI mice post-dose with LLB2-10-8 variants: (A) plasma PK, (B) brain PK, (C) parenchymal fraction, (D) vasculature fraction, (E) cell associated fraction, and (F) non-cell associated fraction.

FIG. 35 shows immunohistochemistry for huIgG on brain sections from CD98hc^{mu/hu} KI mice 1, 7, 14 and 21 days post 50mpk dose of LLB2-10-8 variants.

FIGS. 36A-36C show immunohistochemistry for huIgG and CNS cell type markers on brain sections from CD98hc^{mu/hu} KI 7 days post 50mpk dose of LLB2-10-8 variants: (A) Iba1 for microglial, (B) AQP4 for astrocyte processes, and (C) NeuN for neurons.

FIGS. 37A-37C show (A) plasma PK, (B) brain PK, and (C) Abeta reduction (PD) with CD98hc TVs with BACE1 Fabs.

FIGS. 38A and 38B show immunohistochemistry for huIgG, NeuN (neurons), and LAMP2 (lysosomes) on brain sections from CD98hc^{mu/hu} KI 7 days post-dose of CD98hc TVs with BACE1 Fabs.

FIGS. 39A-39D show huIgG PK in plasma and whole brain lysate in mice dosed at 15mpk: (A) plasma PK for monvalent variants, (B) brain PK for monvalent variants, (C) plasma PK for bivalent variants), and (D) brain PK for bivalent variants.

FIGS. 40A-40F show plasma and brain exposure in NHP and capillary depletion results in NHP brain tissue: (A) plasma exposure, (B) brain exposure, (C) brain parenchymal fraction, (D) vasculature fraction, (E) cell associated fraction, and (F) non-cell associated fraction.

FIGS. 41A-41C show immunohistochemistry for huIgG and CNS cell type markers on NHP brain sections: (A) Iba1 for microglial, (B) AQP4 for astrocyte processes, and (C) NeuN for neurons.

FIGS. 42A and 42B illustrate binding epitopes for CD98hc-binding molecules: (A) LLB2 family and (B) LLB1 family.

FIGS. 43A and 43B show quantification of cell uptake at 37° C. of clone 1 with LALA, clone 3 with LALA, and controls into HEK293T human TfR positive cells (FIG. 43A) and Chinese hamster ovary (CHO) cells ectopically expressing cynomolgus monkey TfR (FIG. 43B).

FIG. 44 shows the Plasma PK of clone 1 with LALA and clone 3 with LALA.

FIGS. 45A and 45B show the concentrations of monovalent clone 1-112_L, monovalent clone 1-112_LS, monovalent clone 1-292, monovalent clone 1-321, and controls in whole brain (FIG. 45A) and plasma (FIG. 45B) of chimeric huTfRapical knock-in mice at 24 hours after administering the mice with 50 mg/kg dose of clone or control.

FIG. 46 shows a multidose study of clones and controls dosed at 50 mg/kg at day 0, 3, and 5 in chimeric huTfRapical knock-in mice at 24 hours after the final dose, showing brain Aβ40 levels.

FIGS. 47A-47C show structure of clone 6.5.11.5.42 with TfR apical domain and modeled full length human TfR domain (FIG. 47A); zoom of FIG. 47A that shows the clash between human TfR domains (FIG. 47B); structure of clone 1-112 having LALA and M428L with TfR apical domain and modeled full length human TfR domain (FIG. 47C).

### DETAILED DESCRIPTION

### I. Introduction

We have developed a number of approaches for generating non-native binding sites in polypetides by screening polypeptide libraries for novel binders. One challenge in introducing non-native binding sites is that such libraries often contain a large number of sequences that have undesired properties (e.g., non-specific binding or lack of developability). As described below, the “limited liability” approach can reduce the frequency of amino acids that are associated with these undesired properties, thus resulting in libraries that produce a greater number of useful sequences. This limited liability approach can be used in a variety of protein scaffolds, including the immunoglobulins (i.e., in both the CDR and non-CDR portions) and other scaffolds such as fibronectin or any other protein scaffold described herein, to enhance and accelerate the discovery of novel polypeptide binders. In certain instances, it can be used in libraries where the engineered portion of the polypeptide includes an exposed side of a beta-sheet within the polypeptide, as described below.

We have also developed immunoglobulin libraries that have been engineered with modifications in a beta-sheet surface. These libraries have been used to generate novel binding sites in the non-CDR portion of the immunoglobulin, and specifically have been used to generate novel molecules that bind to the CD98 heavy chain (CD98hc) and the transferrin receptor (TfR). The disclosure is based, in part, on the discovery that certain amino acids, particularly those at beta-sheet positions in the CH3 domain of an Fc polypeptide, can be substituted to generate a modified CH3 domain containing a novel binding site specific for CD98hc (e.g., a CD98hc-binding site). Beta-sheet positions in the CH3 domain include positions 347-351, 363-372, 378-383, 391-393, 406-412, 422-428, and 437-441, according to EU numbering and other beta-sheet residues in constant domains that are described herein, e.g., in Table 1B. Substituting amino acids at beta-sheet positions can offer several advantages in generating an immunoglobulin domain containing a non-native binding site. First, the beta-sheet surface in the domain is stable and allows for diversity in the amino acid substitutions at the surface without disrupting the domain structure fold. In some embodiments, the amino acid substitutions are located on the solvent-exposed side of the domain beta-sheet surface. Second, making amino acid substitutions at beta-sheet positions avoids changing the flexible loop regions in the domain, which, in some cases, can introduce undesired conformational flexibility. Moreover, the concave surface of the beta-sheet structure in the domain is ideal for forming protein-protein interactions, the beta-sheet structure is also distinct from the FcRn and FcγR binding site in the CH3 domain.

The engineered approaches described herein have been used to discover particular polypeptides that bind to CD98hc or TfR. These polypeptides are transcytosed across the blood-brain barrier in mammals, as described herein. CD98 is highly expressed on brain endothelial cells and therefore a promising target for receptor mediated transcytosis (RMT). CD98 is a heterodimer formed between CD98hc (4F2 heavy chain) and a CD98 light chain. To date six CD98 light chains have been identified, i.e., LAT1 (SLC7A5, 4F2 light chain), LAT2 (SLC7A8), y⁺LAT1 (SLC7A7), y⁺LAT2 (SLC7A6), Asc-1 (SLC7A10), or xCT (SLC7A11). In complex, CD98 heavy chain transports the light chain to the cell surface where it functions as a large neutral amino acid transporter which preferentially transports branched-chain (valine, leucine, isoleucine) and aromatic (tryptophan, tyrosine, phenylalanine) amino acids. Leveraging the CD98 receptor-mediated transcytosis pathway, the polypeptides containing a CD98hc-binding site described herein can be used to transport therapeutic agents across the BBB. This approach can substantially improve brain uptake of the therapeutic agents and is therefore highly useful for treating disorders and diseases where brain delivery is advantageous. In addition, this approach can be used to provide brain uptake and delivery to specific extracellular or neuro-oncology targets in the brain. For example, CD98hc-binding polypeptides provided herein may be used to target such extracellular targets or neuro-oncology targets while retaining wild-type effector function, if so desired. In addition, such CD98hc-binding polypeptides provided herein may be used to target such extracellular targets in cases where neuronal uptake is undesireable (e.g., the target is an antigen or plaque such as Abeta, Tau or alpha-synuclein). The CD98hc-binding polypeptides provided herein have distinct kinetic, biodistribution, and safety properties that may provide optimized and fit-for-purpose BBB transport platforms for protein-based therapeutics.

Also described herein are polypeptides that bind a transferrin receptor (TfR). TfR is highly-expressed on the blood-brain barrier (BBB) and naturally moves transferrin from the blood into the brain. Taking these advantages already offered by TfR, the polypeptides containing a TfR-binding site described herein can be used to transport therapeutic agents across the BBB. This approach can substantially improve brain uptake of the therapeutic agents and is therefore highly useful for treating disorders and diseases where brain delivery is advantageous.

Also provided herein are methods of generating polypeptides comprising modified CH3 domains that bind to CD98hc or TfR. A polypeptide comprising a modified CH3 domain described herein can be analyzed for CD98hc binding or TfR binding and further mutated to enhance binding as described herein.

In a further aspect, also provided herein are treatment methods and methods of using a CD98hc-binding or TfR-binding polypeptide to target a composition to CD98hc-expressing or TfR-expressing cells, e.g., to deliver the composition to that cell, or to deliver a composition across an endothelium such as the BBB.

### II. Definitions

As used herein, the singular forms “a,” “an,” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range indicate that the numeric value as well as reasonable deviations from the value known to the skilled person in the art, for example ±20%, ±10%, or ±5%, are within the intended meaning of the recited value.

As used herein, the term “CD98hc” or “CD98 heavy chain” refers to 4F2 cell-surface antigen heavy chain and is encoded by the SLC3A2 gene. CD98hc is also known as 4F2 heavy chain. The human CD98hc sequence is set forth in SEQ ID NO:55 and UNIPROT Accession No. P08195. CD98hc sequences from other species are also known (e.g., mouse, UNIPROT Accession No. P10852 and cynomolgus monkey, UNIPROT Accession No. G8F3Z0).

As used herein, the term “transferrin receptor” or “TfR” refers to transferrin receptor protein 1. The human transferrin receptor 1 polypeptide sequence is set forth in SEQ ID NO:127. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain.

As used herein, the terms “CH3 domain” and “CH2 domain” refer to immunoglobulin constant region domain polypeptides. For purposes of this application, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:4).

As used herein, the terms “wild-type,” “native,” and “naturally occurring” as used with reference to a CH3 or CH2 domain, refer to a domain that has a sequence that occurs in nature.

As used herein, the term “mutant,” as used with reference to a mutant polypeptide or mutant polynucleotide, is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

As used herein, the term “amino acid” refers to naturally occurring and synthetic amino acids, as well as amino acid analogs and amino acid mimetics that function in a manner similar to the naturally occurring amino acids. Naturally occurring are those encoded by the genetic code, as well as those amino acids that are later modified, e.g., hydroxyproline, γ-carboxyglutamate and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

As used herein, “limited diversity,” in the context of a randomized codon within a polynucleotide library or any amino acid position within a polypeptide library described herein, refers to a codon or position that is restricted to allow fewer than all 20 naturally occurring amino acids.

As used herein, “beta-sheet position” in the context of a polypeptide is meant an amino acid that falls within a portion of the polypeptide whose structure is predominantly beta-sheet.

As used herein, the term “immunoglobulin-like fold” refers to a protein domain of between about 80-150 amino acid residues that includes two layers of antiparallel beta-sheets, and in which the flat, hydrophobic faces of the two beta-sheets are packed against each other.

As used herein, the terms “polypeptide” and “peptide” are used interchangeably to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

As used herein, the term “constant domain” refers to a domain in the constant region of an immunoglobulin molecule (e.g., CH1, CH2, CH3, CH4, Ckappa, Clambda).

As used herein, the term “modified constant domain” refers to a constant domain that has at least one mutation, e.g., a substitution, deletion or insertion, as compared to a wild-type immunoglobulin constant domain sequence, but retains the overall Ig fold or structure of the native constant domain.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region, but does not contain a variable region.

As used herein, the term “protein” refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

As used herein, the terms “identical” or percent “identity,” in the context of two or more polypeptide sequences, refer to two or more sequences or subsequences that are the same or have a specified percentage of amino acid residues, e.g., at least 60%, 65%, 70%, 75%, 80%, 85%, 90%, or 95% or greater, that are identical over a specified region when compared and aligned for maximum correspondence over a comparison window or designated region as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

As used herein, the term “binding affinity” refers to the strength of a non-covalent interaction between two molecules, e.g., between a Fab or scFv and an antigen, or between a polypeptide described herein (or a target-binding portion thereof) and a target. Thus, for example, the term may refer to 1:1 interactions between a Fab or scFv and an antigen or between a polypeptide described herein (or a target-binding portion thereof) and a target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the FortéBio® Octet platform). As used herein, “binding affinity” includes not only formal binding affinities, such as those reflecting 1:1 interactions between a Fab or scFv and an antigen or between a polypeptide described herein (or a target-binding portion thereof) and a target, but also apparent affinities for which K_{D}'s are calculated that may reflect avid binding.

As used herein, the term “specifically binds” refers to a molecule (e.g., a Fab, an scFv, or a polypeptide described herein (or a target-binding portion thereof) that binds to an epitope or target with greater affinity, greater avidity, and/or greater duration to that epitope or target in a sample than it binds to another epitope or non-target compound (e.g., a structurally different antigen). In some embodiments, a Fab, scFv, or polypeptide described herein (or a target-binding portion thereof) that specifically binds to an epitope or target is a Fab, scFv, or polypeptide described herein (or a target-binding portion thereof) that binds to the epitope or target with at least 5-fold greater affinity than other epitopes or non-target compounds, e.g., at least 6-fold, 7-fold, 8-fold, 9-fold, 10-fold, 25-fold, 50-fold, 100-fold, 1000-fold, 10,000-fold, or greater affinity. The term “specific binding,” “specifically binds to,” or “is specific for” a particular epitope or target, as used herein, can be exhibited, for example, by a molecule having an equilibrium dissociation constant K_{D} for the epitope or target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that a Fab or scFv that specifically binds to a target from one species may also specifically bind to orthologs of that target.

As used herein, the terms “subject,” “individual,” and “patient” are used interchangeably to refer to a mammal, including but not limited to humans, non-human primates, rodents (e.g., rats, mice, and guinea pigs), and other mammalian species. In one embodiment, the patient is a human.

As used herein, the terms “treatment,” “treating,” and the like generally mean obtaining a desired pharmacologic and/or physiologic effect. “Treating” or “treatment” may refer to any indicia of success in the treatment or amelioration of a neurodegenerative disease (e.g., Alzheimer's disease or another neurodegenerative disease described herein), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

As used herein, the term “pharmaceutically acceptable excipient” refers to a non-active pharmaceutical ingredient that is biologically or pharmacologically compatible for use in humans or animals, such as, but not limited to a buffer, carrier, or preservative.

As used herein, the term “therapeutic agent” refers to any molecule, drug, or agent that is used in the treatment and/or prevention of a disease. A therapeutic agent can be an organic small molecule or compound, a polypeptide, a protein, a nucleic acid, and/or a combination of any of the above. In some embodiments, a therapeutic agent can be a known molecule, drug, or agent. In some embodiments, the therapeutic agent is a polypeptide containing an antigen-binding domain, e.g., an antibody variable domain polypeptide having one or more complimentarity determining regions (CDRs), or an antigen-binding fragment thereof. In particular embodiments, a therapeutic agent can be a Fab (e.g., a Fab that binds to a target that is not a TfR or CD98hc). In some embodiments, depending on the disease to be treated, a therapeutic agent can bind to a target (e.g., a biological target, a therapeutic target, a target that is not a TfR or CD98hc) to treat and/or prevent the disease. Such targets may include cell surface targets in the brain, such as on a microglial cell, an astrocyte, an oligodendrocyte, a neuron, and a cancer cell. For example, such targets include TREM2, PILRA, CD33, CR1, ABCA1, ABCA7, MS4A4A, MS4A6A, MS4A4E, HLA-DR5, HLA-DR1, IL1RAP, TREML2, IL-34, SORL1, ADAM17, and Siglec11. In some embodiments, the target may include alpha-synuclein or derivatives or fragments thereof, amyloid-beta peptide or derivatives of fragments thereof, Tau or derivatives or fragments thereof, pTau, huntingtin, transthyretin, or TAR DNA-binding protein 43 (TDP-43) or derivatives or fragments thereof.

In some embodiments, the target is on a tumor cell and is selected from the group consisting of ALK, AXL, CD25, CD44v6, CD46, CD56 (NCAM), CDH6 (cadherin 6), CEACAM 5 (CD66E), EGFR, EGFR viii, ETBR, FGFR (1-4), Folate Receptor alpha, GAL-3BP (galectin binding protein), GD2, GD3, GloboH (globohexasylceramide), gp100, gpNMB, HER2, HER3, HER4, IGFR1, KIT, LIV1A, LRRC15 (leucine rich repeat containing 15), MET, NaPi2B, PDL1, PMEL17, PRAME, PSMA, PTK7 (CCK4; colon carcinoma kinase), RON, ROR1, TF (tissue factor), and TROP2. In some embodiments the cell is a hematological cancer cell and the cell surface receptor is selected from the group consisting of B7H3, BCMA, CD125, CD166, CD19, CD20, CD205, CD22, CD25, CD30, CD37, CD39, CD73, and CD79b. Known therapeutic agents for the treatment of cancer include, for example, lorlatinib, crizotinib, cabozantinib, basiliximab, daclizumab, bivatuzumab, promiximab, lorvotuzumab, polatuzumab, tusamitamab, sunitinib, cetuximab, panitumumab, nimotuzumab, necitumumab, rindopepimut (CDX-110), amivantamab, pemigatinib, erdafitinib, STRO-002, bevacizumab, naxitamab, ipilimumab, tebentafusp, glembatumumab, margetuximab-cmkb, enhertu, trastuzumab, pertuzumab, patritumab, seribantumab, lumretuzumab, elgemtumab, U3-1402, AV-203, KTN3379, AVE1642, MK-0646, cixutumumab, ladiratuzumab, gemtuzumab, pembrolizumab, sacituzumab, samrotamab, amivantamab-vmjw, TEPMETKO, lifastuzumab, ¹⁷⁷lutetium-PSMA-617, cofetuzumab, Zt/g4-MMAE, VLS-101, brexucabtagene, CS5001, tisotumab, sacituzumab, teclistamab, atezolizumab, avelumab, cosibelimab, durvalumab, belantamab, benralizumab, tafasitamab, loncastuximab, obinutuzumab, ofatumumab, rituximab, MEN1309/OBT076, inotuzumab, and brentuximab.

Additional known targets in the brain, as well as agents that bind such targets, are described in the following references which are hereby incorporated by reference herein: WO 2016/023019; WO 2017/062672; WO 2018/195506; WO 2019/118513; WO 2019/023292; WO 2019/079529; WO 2019/180224; US2019/0040130; US2019/0174730; WO 2020/069050; US2017/0137518; US2012/0258110; WO 2019/126472; U.S. Pat. No. 8,691,227; WO 2019/152715; US 2007/026425; WO 2019/028283; US2018/016066, U.S. Pat. No. 9,079,958; WO 2020/069050; WO 2022/258841; J Immunol 2000 165:1197-1209; Translational Neurodegeneration, 11, 18 (2022).

As used herein, a “therapeutic amount” or “therapeutically effective amount” of an agent is an amount of the agent (e.g., any of the proteins described herein) that treats a disease in a subject.

As used herein, term “administer” refers to a method of delivering agents, compounds, or compositions to the desired site of biological action. These methods include, but are not limited to, topical delivery, oral delivery, parenteral delivery, intravenous delivery, intradermal delivery, intramuscular delivery, intrathecal delivery, colonic delivery, rectal delivery, or intraperitoneal delivery. In one embodiment, a protein as described herein is administered intravenously.

### III. Polypeptide Engineering

We have developed a “limited liability” design approach for polypeptide libraries, as well as libraries in polypeptides, particularly immunoglobulin molecules, that include a substantial beta-sheet component. These are detailed in the sections below. Also described are engineering methods that can be used with these libraries and library design approach to generate polypeptides with non-native binding sites, including for example sites that bind to CD98hc or TfR.

Libraries with Limited Liability

We have observed that large (>9 positions) combinatorial libraries in polypeptides, when used to screen against potential targets, produce a significant number of polypeptides that bind non-specifically (e.g., through hydrophobic interactions) or have a liability that makes them difficult to work with (e.g., express poorly, overly hydrophobic, poor stability). To reduce but not eliminate the appearance of amino residues that are associated with these properties in our libraries, we have taken what we have termed a “limited liability” approach. This approach involves reducing the frequency at which certain amino acids (e.g., Cys, Trp, Met, Arg, and Gly) appear in the library, but not eliminating their presence altogether, while also maintaining variabilty at these limited positions, for example, allowing for at least 8, 10, 12, 14, 15, or 16 amino acids at these positions. Specifically, this involves reducing the appearance of at least one of these amino acids at 10⁻⁶⁰% (e.g., 20-60%, 30%-60%, or 40-60%) of the randomized positions in the library, particularly where some, or even all, of the other randomized positions allow for all twenty naturally occuring amino acids. In particular cases, the positions with limited diversity alternate with positions that allow all twenty amino acids. This can avoid having too many amino acids that can contribute to undesired properties in close proximity. In some cases, the alternating is positioned relative to the primary sequence of the polypeptide. Where the structure of the protein is known (e.g., if the crystal structure has been solved), placement of the limited liability positions can be spaced out relative to the positions with greater or full diversity in three-dimensional space. As explained in the examples below, this approach has led to discovery of the specific CD98hc-binding polypeptides described herein.

Limited liability libraries can be generated using any known approach for peptide library development. The libraries described in the examples herein were generated from polynucleotide libraries encoding for the polypeptides of interest using degenerate codons, in particular using the NNK codon (which allows all 20 amino acids) interspersed with limited liability codons, such as NHK, which does not allow Arg, Cys, Trp, or Gly. The invention also contemplates using other codons that provide a limited liability advantage. Possible codons can be selected from any known that provide “limited liability,” such as those described in Mena et al., Protein Eng Des Sel 18:559-61, 2005, which are shown below. Table II in Mena et al. is shown as Table 1A below.

TABLE 1A
Degenerate codons computed by LibDesign at each
position, from most-inclusive to least inclusive*

| | |
|---|---|
| Pos 1 | |
| NNK | ACDEFGHIKLMNPQRSTVWXY |
| NHK | ADEFHIKLMNPQSTVXY |
| DYK | AFILMSTV |
| WTK | FILM |
| TTC | F |
| Pos2 | |
| NDK | CDEFGHIKLMNQRSVWXY |
| HDK | CFHIKLMNQRSWXY |
| MWK | HIKLMNQ |
| CAC | H |
| Pos3 | |
| NHK | ADEFHIKLMNPQSTVXY |
| MHK | HIKLMNPQT |
| MMC | HNPT |
| AMC | NT |
| ACA | T |
| Pos4 | |
| VNK | ADEGHIKLMNPQRSTV |
| NHK | ADEFHIKLMNPQSTVXY |
| RNK | ADEGIKMNRSTV |
| WYG | LMST |
| ASC | ST |
| AGC | S |
| Pos5 | |
| VNG | AEGKLMPQRTV |
| DBG | AGLMRSTVW |
| DYG | ALMSTV |
| DTG | LMV |
| RTG | MV |
| GTA | V |
| Pos6 | |
| VWK | DEHIKLMNQV |
| VWC | DHILNV |
| KTA | LV |
| GTA | V |
| Pos7 | |
| TTC | F |
| Pos8 | |
| MDK | HIKLMNQRS |
| MWK | HIKLMNQ |
| AWK | IKMN |
| ATK | IM |
| ATA | I |
| Pos9 | |
| MWG | KLMQ |
| MTG | LM |
| CTA | L |
| Pos10 | |
| RNK | ADEGIKMNRSTV |
| RBG | AGMRTV |
| RKG | GMRV |
| RTG | MV |
| GTA | V |

*Residues given in boldface are in the input sequence set, underlined residues are the required wild-type amino acids.

In addition to codon-based techniques or generating libraries based on degenerate codons, limited liability libraries can also be generated using trinucleotide mutagenesis technology. These approaches involve high throughput technologies whereby specific proportions of trinucleotide base pairs each encoding for a single amino acid can be added into the libraries to precisely control the ratio of amino acids at a given position. Technology using such approaches are commercially available from companies such as Sloning BioTechnology GmbH (Germany) and Azenta Life Sciences (Chelmsford, Mass.). These polynucleotide libraries can be expressed to generate polypeptide libraries useful for screening against targets, including TfR and CD98hc.

### Beta-Sheet Libraries

Libraries that include randomzied amino acids within the beta-sheet secondary structure of a polypeptide are also described herein. In general, these libraries use an exposed portion of a beta-sheet, where the randomzied amino acids together form a surface that is capable of creating an antigen binding site. In addition to the beta-sheet surface, the antigen binding site can also include residues from adjacent areas on the polypeptide, e.g., in loop regions that connect the beta-strands or in other structural features of the protein that are proximate in three-dimensional space.

Use of beta-sheet regions has certain advantages, including those described herein, which include greater structural stability (as compared to loop regions or other less structured portions of the polypeptide) of the antigen binding site, and in certain contexts, the formation of distinct surface topologies (e.g., a flat, extended concave surface) well-suited for forming some protein-protein interactions.

Specific examples of beta-sheet libraries include those generated from beta-sheet portions of immunoglobulin proteins. In some examples, the beta-sheet libraries are generated in a constant domain of the immunoglobulin, for example in a CH1, CH2, CH3, CH4, or CL domain. Other examples include beta-sheet portions of the variable domains, which can include non-CDR portions of the variable region.

For constant domains of human IgG1 molecules, the positions shown in Table 1B are useful for generating beta-sheet libraries.

TABLE 1B
Surface accessible beta-sheet positions in IgG1 heavy chain constant domains

| | | Surface accesible beta-sheet |
|---|---|---|
| | Surface accesible | positions that are not buried at |
| | beta-sheet positions | a protein-protein interface (EU |
| Domain | (EU numbering) | numbering) |
| CH1 | 124, 126, 128, 139, 141, 143, 145, 147, | 155, 157, 199, 201, 203, 208, 210, |
| | 155, 157, 179, 181, 183, 185, 187, 199, | 212, 214 |
| | 201, 203, 208, 210, 212, 214 | |
| CH2 | 239, 241, 243, 258, 260, 262, 264, 274, | 239, 241, 243, 258, 260, 262, 264, |
| | 276, 278, 301, 303, 305, 307, 320, 322, | 274, 276, 278, 301, 303, 305, 307, |
| | 324, 333, 335 | 320, 322, 324, 333, 335 |
| CH3 | 347, 349, 351, 362, 364, 366, 368, 370, | 347, 362, 378, 380, 382, 411, 424, |
| | 378, 380, 382, 405, 407, 409, 411, 424, | 426, 428, 436, 438, 440 |
| | 426, 428, 436, 438, 440 | |

Based on these positions in the IgG1 heavy chain constant region, corresponding positions can be identified in different domains (e.g., the variable region and light chains), in different subtypes (e.g., IgG2, IgG3, IgG4), different species (e.g., mouse, rat, cynomolgous monkey), and other Ig types (e.g., IgA, IgM, IgE). As an example, alignment of the primary amino acid sequence from different domains to the corresponding domain in the IgG1 heavy chain constant region can be used to determine analogous positions useful for generating beta-sheet libraries in additional domains. Alternatively, structural alignment of a domain to one or more of the Ig domain structures in the IgG1 heavy chain constant region can be used to determine potential beta-sheet library positions in domains for which structural information exists or can be predicted. Whether an identified residue is surface exposed and amenable to inclusion in the library, or buried at a protein-protein interface (e.g. the CH3-CH3 interface, the VH-VL interface) can similarly be determined using structural information about the specific domain, which can be found in databases such as the Protein Data Bank (Berman et al., Nucleic Acids Res, 28: 235-242, 2000) or based on predictions such as the AlphaFold Protein Structure Database (Jumper et al., Nature, 596: 583-589, 2021).

### Protein Scaffolds For Use In Libraries

The limited liability approach to library design and diversification and libraries comprising beta-sheet secondary structure described herein can be used to generate libraries on any appropriate polypeptide scaffold. These can encompass any polypeptide that has beta-sheet secondary structure, which includes the immunoglobulins, fibronectin type-III domains, anticalins, kunitz domains, nanofitins, centyrins, affimers, and lipocalins, as well as numerous other proteins that have this canonical beta-sheet structure.

### Generating Binding Proteins From Polypeptide Libraries

As described below, we have used beta-sheet polypeptide libraries that may, in some cases, employ the limited library concept to discover polypeptides that have been engineered to bind target proteins, specifically CD98hc or TfR.

In general terms, the polypeptide libraries are expressed (e.g., on the cell surface) and interrogated for binding to the target protein. This can be done in any appropriate way, and various aspects of screening approaches are described in Kariolis et al., Sci Transl Med 12(545):eaay1359, 2020. In one approach, the polypeptide library is expressed as a surface display library (e.g., phage display or yeast display), and is incubated with the target protein, which can be can conjugated to magnetic bead (MACS) or fluorescently labeled to facilitate library selections using fluoresnce-activated cell sorting (FACS). Following incubation with the target antigen, binders are separated from non-binders, and this process is repeated to enrich the library for desired polypeptide clones that interact with the target antigen.

Following the identification of initial binders from the polypeptide libraries, improvements to various biochemical and biophysical properies can be further engineered. Some of these improvements can include, but are not limited to stronger, binding to the antigen, specificity (e.g., binding to cynomolgus and human forms of the antigen), or an increase in structural (e.g., thermal) stability. To achieve this, maturation libraries (e.g., as described herein) can be designed and screened to isolate variants with the desired improved properties. Approaches for designing these libraries can include: expanding the epitope by mutating amino acid positions in proximity to the original library positions, randomizing the sequence of initial binders using an approach biased towards keeping some part of the original sequence, or using error-prone PCR to randomly incorporate mutations across the domain to explore additional sequence space both within and proximal to the binding epitope. These libraries are then screened using the methods described above to isolate clones with a desired set of properties.

### IV. CD98 Heavy Chain Binding Polypeptides

This section describes generation of polypeptides in accordance with the present disclosure that bind to a CD98hc protein (i.e., polypeptides having a CD98hc-binding site). These polypeptides are capable of being transported across the blood-brain barrier (BBB).

A polypeptide as provided herein can comprise a modified CH3 domain that specifically binds to a CD98hc protein. As described herein, when describing a polypeptide (e.g., an Fc polypeptide) comprising a modified CH3 domain comprising amino acids 111-217 of certain SEQ ID NO(S), or a modified CH3 domain comprising amino acid substitutions or deletions relative to amino acids 111-217 of certain SEQ ID NO(S), or a modified CH3 domain comprising a sequence having a percent identity to amino acids 111-217 of certain SEQ ID NO(S), such descriptions are directed to the sequence of the modified CH3 domain, and are not to be construed as limiting the polypeptide to contain amino acids 1-110 of the recited SEQ ID NO(S).

One of skill understands that the CH3 domains of other immunoglobulin isotypes, e.g., IgM, IgA, IgE, IgD, etc. may be similarly modified by identifying the amino acids in those domains that correspond to the amino acid substitutions at the positions described herein. Modifications may also be made to corresponding domains from immunoglobulins from other species, e.g., non-human primates, monkey, mouse, rat, or other non-human mammals.

### CD98hc-Binding Site Modifications

In one embodiment, provided herein is a modified polypeptide comprising a modified constant domain (e.g., a modified CH3 domain) that specifically binds to a CD98hc protein wherein the modified constant domain comprises at least five, six, seven, eight, or nine substitutions in a set of amino acid positions consisting of 382, 384, 385, 387, 422, 424, 426, 438, 440; and wherein the substitutions are determined with reference to SEQ ID NO:1 and the positions are determined according to EU numbering.

In some embodiments, polypeptides that bind to CD98hc are from the LLB2 family. In some embodiments, the polypeptides comprise at least eleven, twelve, thirteen, fourteen, or fifteen substitutions in a set of amino acid positions consisting of 378, 380, 382, 383, 384, 385, 386, 387, 389, 391, 421, 422, 424, 426, 428, 434, 436, 438, 440, 441, and 442. In some embodiments, the substitutions are selected from a S, V, D, E, or Y at position 378, a L, I, M, A, Q, V, or K at position 380, a N, S, L, M, P, Y, K, A, or T at position 382, a T, F, N, P, D, L, H, or Q at position 383, a K, R, H, I, L, F, Y, V, or Q at position 384, a F or Y at position 385, a V, L, A, I, F, Y, S, T, H, R, or E at position 386, a L or I at position 387, a D, Q, A, T, H, or V at position 389, a T, V, or A at position 391, a E, Q, or A at position 421, a L, M, I, T, or P at position 422, an A at position 424, a N at position 426, a L, T, P, Y F, I, A, K, H, or W at position 428, a S at position 434, a L, V, H, F, P, R or W at position 436, a F or W at position 438, a L, P, E, N, V, A, I, or D at position 440, a P at position 441, and an A, V, M, Q, F, P, L, Y, K, R, H, or M at position 442.

In some embodiments, polypeptides that bind to CD98hc are from the LLB1 family. In some embodiments, the polypeptides comprise at least eight, nine, ten, eleven, twelve, thirteen, fourteen, fifteen, sixteen, seventeen, eighteen, or nineteen substitutions in a set of amino acid positions consisting of 378, 380, 382, 383, 384, 385, 386, 387, 389, 421, 422, 424, 426, 428, 434, 436, 438, 440, and 442. In some embodiments, the substitutions are selected from a S or V at position 378, a D, M, N, P, F, or H at position 380, a R, Y, F, S, W, Y, K, or N at position 382, a T at position 383, a L, Y, A, S, or F at position 384, a F, K, D, M, I, N, Y, L, or H at position 385, a T, P, E, K, A, V, D, T, or F at position 386, a N, L, Y, R, F, G, S, D, or T at position 387, a T, Y, or F at position 389, a D, E, or Q at position 421, a I, K, L, R, T, F, or H at position 422, a V, W, G, L, I, P, or Y at position 424, a D, A, Q, W, L, or P at position 426, a L or Y at position 428, a S at position 434, a F at position, 436, a I, V, F, N, P, or S at position 438, and a K, T, P, I, or F at position 440, and a Q or M at position 442.

In one embodiment, a modified polypeptide comprising a modified constant domain (e.g., a modified CH3 domain) comprises a sequence having at least 80%, 85%, 90%, or 95% sequence identity to amino acids 111-217 of the sequence of any one of SEQ ID NOS:28-45.

### V. Transferrin Receptor-Binding Polypeptides

This section describes generation of polypeptides in accordance with the present disclosure that bind to a transferrin receptor (TfR) (i.e., polypeptides having a TfR-binding site). These polypeptides are capable of being transported across the blood-brain barrier (BBB).

A polypeptide as provided herein can comprise a modified CH3 domain that specifically binds to a TfR. As described herein, when describing a polypeptide (e.g., an Fc polypeptide) comprising a modified CH3 domain comprising amino acids 111-217 of certain SEQ ID NO(S), or a modified CH3 domain comprising amino acid substitutions and/or deletions relative to amino acids 111-217 of certain SEQ ID NO(S), or a modified CH3 domain comprising a sequence having a percent identity to amino acids 111-217 of certain SEQ ID NO(S), such descriptions are directed to the sequence of the CH3 domain, and are not to be construed as limiting the polypeptide to contain amino acids 1-113 of the recited SEQ ID NO(S).

One of skill understands that the CH3 domains of other immunoglobulin isotypes, e.g., IgM, IgA, IgE, IgD, etc. may be similarly modified by identifying the amino acids in those domains that correspond to the amino acid substitutions at the positions described herein. Modifications may also be made to corresponding domains from immunoglobulins from other species, e.g., non-human primates, monkey, mouse, rat, or other non-human mammals.

### TfR-Binding Site Modifications

In one embodiment, provided herein is a polypeptide comprising a modified CH3 domain that specifically binds to a transferrin receptor (TfR), wherein the modified CH3 domain comprises five, six, or seven amino acid substitutions in a set of amino acid positions comprising 422, 424, 426, 433, 434, 438, and 440. The modified CH3 domain may not have the combination of G at position 437, F at position 438, and D at position 440, and wherein the positions are determined according to EU numbering.

Also provided herein is a polypeptide comprising a modified CH3 domain that specifically binds to a transferrin receptor (TfR), wherein the modified CH3 domain comprises three, four, five, six, seven, or eight amino acid substitutions and/or one or two amino acid deletions in a set of amino acid positions comprising 380 and 382-389; and five, six, or seven amino acid substitutions in a set of amino acid positions comprising 422, 424, 426, 433, 434, 438, and 440, wherein the positions are determined according to EU numbering.

Also provided herein is a polypeptide comprising a modified CH3 domain that specifically binds to a transferrin receptor (TfR), wherein the modified CH3 domain comprises a sequence comprising at least one amino acid substitution in the sequence of VFSCSVMHEALHNHYTQKS (SEQ ID NO:57), wherein the sequence of SEQ ID NO:57 is from position 422 to position 440 of an Fc polypeptide (e.g., SEQ ID NO:1), the sequence does not have the combination of G at position 437, F at position 438, and D at position 440, and the positions are determined according to EU numbering. In some embodiments, the modified CH3 domain comprises a sequence comprising five, six, or seven amino acid substitutions in a set of amino acid positions comprising 422, 424, 426, 433, 434, 438, and 440.

Also provided herein is a polypeptide comprising a modified CH3 domain that specifically binds to a transferrin receptor (TfR), wherein the modified CH3 domain comprises a first sequence comprising at least one amino acid substitution and/or deletion in the sequence of AVEWESNGQPENN (SEQ ID NO:56), and a second sequence comprising at least one amino acid substitution in the sequence of VFSCSVIHEALHNHYTQKS (SEQ ID NO:57), wherein the sequence of SEQ ID NO:56 is from position 378 to position 390 of an Fc polypeptide (e.g., SEQ ID NO:1), the sequence of SEQ ID NO:57 is from position 422 to position 440 of an Fc polypeptide (e.g., SEQ ID NO:1), and the positions are determined according to EU numbering. In some embodiments, the modified CH3 domain comprises three, four, five, six, seven, or eight amino acid substitutions in a set of amino acid positions comprising 380 and 382-389. In certain embodiments, the modified CH3 domain comprises five, six, or seven amino acid substitutions in a set of amino acid positions comprising 422, 424, 426, 433, 434, 438, and 440.

### Modifications at Positions 380 and 382-389

Provided herein are polypeptides that comprise a modified CH3 domain having at least one (e.g., one, two, three, four, five, six, seven, or eight (e.g., three, four, five, six, seven, or eight)) amino acid substitution and/or at least one (e.g., one or two) amino acid deletion in a set of amino acid positions comprising 380 and 382-389, according to EU numbering. The modified CH3 domain can comprise a sequence comprising at least one (e.g., one, two, three, four, five, six, seven, or eight (e.g., three, four, five, six, seven, or eight)) amino acid substitution and/or at least one (e.g., one or two) amino acid deletion in the sequence of AVEWESNGQPENN (SEQ ID NO:56), which is from position 378 to position 390 of an Fc polypeptide (e.g., SEQ ID NO:1). In some embodiments, the modified CH3 domain can comprise a sequence comprising at least one (e.g., one, two, three, four, five, six, seven, or eight (e.g., three, four, five, six, seven, or eight)) amino acid substitution and/or at least one (e.g., one or two) amino acid deletion in a set of amino acid positions comprising 380 and 382-389 relative to the sequence of SEQ ID NO:56, in which the positions are numbered according to EU numbering.

In some embodiments, the modified CH3 domain in the polypeptide comprises F at position 382. In certain embodiments, the modified CH3 domain comprises A or a polar amino acid at position 383. In particular embodiments, the modified CH3 domain comprises A at position 383. In certain embodiments, the modified CH3 domain comprises a polar amino acid (e.g., Y, S, N, Q, T, H, K, D, E, or W (e.g., Y or S)) at position 383. In certain embodiments, the modified CH3 domain comprises Y or S at position 383. In some embodiments, the modified CH3 domain comprises G, N, or an acidic amino acid at position 384. In some embodiments, the modified CH3 domain comprises G or N at position 384. In some embodiments, the modified CH3 domain comprises an acidic amino acid (e.g., D or E) at position 384. In some embodiments, the modified CH3 domain comprises N, R, or a polar amino acid at position 389. In some embodiments, the modified CH3 domain comprises N or R at position 389. In some embodiments, the modified CH3 domain comprises a polar amino acid (e.g., Y, S, N, Q, T, H, K, D, E, or W (e.g., S or T)) at position 389. In some embodiments, the modified CH3 domain comprises S or T at position 389.

In certain embodiments, at least one of the amino acid substitutions in a set of amino acid positions comprising 380 and 382-389 is at a beta-sheet position relative to the sequence of SEQ ID NO:56. In some embodiments, the modified CH3 domain comprises one, two, or three amino acid substitutions at beta-sheet positions relative to the sequence of SEQ ID NO:56. In particular embodiments, the beta-sheet position(s) are selected from the group consisting of: positions 380, 382, and 383, according to EU numbering. In certain embodiments, the modified CH3 domain comprises an amino acid substitution at position 380 relative to the sequence of SEQ ID NO:56, for example, E, N, F, or Y. In particular embodiments, the amino acid substitution at position 380 is E. In certain embodiments, the modified CH3 domain comprises an amino acid substitution (e.g., F) at position 382 relative to the sequence of SEQ ID NO:56. In certain embodiments, the modified CH3 domain comprises an amino acid substitution at position 383 relative to the sequence of SEQ ID NO:56, for example, Y or A. In particular embodiments, the amino acid substitution at position 383 is Y.

In some embodiments of the polypeptide described herein, the polypeptide can comprise a modified CH3 domain comprising at least one position selected from the following: E, N, F, or Y at position 380, F at position 382, Y, S, A, or an amino acid deletion at position 383, G, D, E, or N at position 384, D, G, N, or A at position 385, Q, S, G, A, or N at position 386, K, I, R, or G at position 387, E, L, D, or Q at position 388, N, T, S, or R at position 389, wherein the positions are numbered according to EU numbering. In particular embodiments, the modified CH3 domain can comprise five, six, seven, or eight positions selected from the following: F at position 382, Y or S at position 383, G, D, or E at position 384, D, G, N, or A at position 385, Q, S, or A at position 386, K at position 387, E or L at position 388, N, T, or S at position 389. In particular embodiments, the modified CH3 domain can comprise the following five positions: F at position 382, E at position 384, S at position 386, K at position 387, and T at position 389. In particular embodiments, the modified CH3 domain can comprise the following five positions: F at position 382, G at position 384, A at position 385, K at position 387, and S at position 389. In particular embodiments, the modified CH3 domain can comprise the following six positions: F at position 382, G at position 384, A at position 385, K at position 387, L at position 388, and T at position 389. In particular embodiments, the modified CH3 domain can comprise the following six positions: F at position 382, Y at position 383, E at position 384, A at position 385, K at position 387, and L at position 388. In particular embodiments, the modified CH3 domain can comprise the following seven positions: F at position 382, Y at position 383, G at position 384, N at position 385, A at position 386, K at position 387, and T at position 389. In particular embodiments, the modified CH3 domain can comprise the following eight positions: F at position 382, Y at position 383, D at position 384, D at position 385, S at position 386, K at position 387, L at position 388, and T at position 389.

Modifications at Positions 422, 424, 426, 433, 434, 438, and/or 440

Provided herein are polypeptides that comprise a modified CH3 domain having at least one (e.g., one, two, three, four, five, six, or seven (e.g., five, six, or seven)) amino acid substitution in a set of amino acid positions comprising 422, 424, 426, 433, 434, 438, and 440, according to EU numbering. The modified CH3 domain can comprise a sequence comprising at least one (e.g., one, two, three, four, five, six, or seven (e.g., five, six, or seven)) amino acid substitution in the sequence of VFSCSVMHEALHNHYTQKS (SEQ ID NO:57), which is from position 422 to position 440 of an Fc polypeptide (e.g., SEQ ID NO:1). In some embodiments, the modified CH3 domain can comprise a sequence comprising at least one (e.g., one, two, three, four, five, six, or seven (e.g., five, six, or seven)) amino acid substitution in a set of amino acid positions comprising 422, 424, 426, 433, 434, 438, and 440 relative to the sequence of SEQ ID NO:57, in which the positions are numbering according to EU numbering. The modified CH3 domain does not have the combination of G at position 437, F at position 438, and D at position 440, wherein the positions are determined according to EU numbering.

In some embodiments of the modified CH3 domain in the polypeptide, at least one of the amino acid substitutions in a set of amino acid positions comprising 422, 424, 426, 433, 434, 438, and 440 is at a beta-sheet position relative to the sequence of SEQ ID NO:57. In some embodiments, the modified CH3 domain comprises at one, two, three, or four amino acid substitutions at beta-sheet positions relative to the sequence of SEQ ID NO:57. In particular embodiments, the beta-sheet position(s) are selected from the group consisting of: positions 424, 426, 438, and 440, according to EU numbering. In certain embodiments, the modified CH3 domain comprises an amino acid substitution at beta-sheet position 424 relative to the sequence of SEQ ID NO:57. The amino acid substitution at beta-sheet position 424 in the modified CH3 domain can be A. In certain embodiments, the modified CH3 domain comprises an amino acid substitution at beta-sheet position 426 relative to the sequence of SEQ ID NO:57. The amino acid substitution at beta-sheet position 426 can be E. In certain embodiments, the modified CH3 domain comprises an amino acid substitution at beta-sheet position 438 relative to the sequence of SEQ ID NO:57. The amino acid substitution at beta-sheet position 438 can be Y. In some embodiments, the modified CH3 domain comprises an amino acid substitution at beta-sheet position 440 relative to the sequence of SEQ ID NO:57. The amino acid substitution at beta-sheet position 440 can be L.

In some embodiments of the modified CH3 domain in the polypeptide, the modified CH3 domain comprises H or E (e.g., H) at position 433. In some embodiments, the modified CH3 domain comprises N or G (e.g., N) at position 434.

In some embodiments of the polypeptide described herein, the polypeptide can comprise a modified CH3 domain comprising at least one position selected from the following: L at position 422, A at position 424, E at position 426, H or E at position 433, N or G at position 434, Y at position 438, and L at position 440. In particular, the modified CH3 domain can comprise five positions selected from the following: L at position 422, A at position 424, E at position 426, Y at position 438, and L at position 440.

### TfR-Binding Polypeptides

The disclosure provides polypeptides comprising a modified CH3 domain that specifically binds to a transferrin receptor (TfR), wherein the modified CH3 domain comprises: (i) a sequence of AVX₁WFX₂X₃X₄X₅X₆X₇X₈N (SEQ ID NO:65), wherein X₁ is E, N, F, or Y; X₂ is Y, S, A, or absent; X₃ is G, D, E, or N; X₄ is D, G, N, or A; X₅ is Q, S, G, A, or N; X₆ is K, I, R, or G; X₇ is E, L, D, or Q; and X₈ is N, T, S, or R; and (ii) a sequence of LFACEVMHEALX₁X₂HYTYKL (SEQ ID NO:67), wherein X₁ is H or E; and X₂ is N or G. The disclosure provides polypeptides comprising a modified CH3 domain that specifically binds to a transferrin receptor (TfR), wherein the modified CH3 domain comprises: (i) a sequence of AVEWFX₁X₂X₃X₄KX₅X₆N (SEQ ID NO:66), wherein X₁ is Y or S; X₂ is G, D, or E; X₃ is D, G, N, or A; X₄ is Q, S, or A; X₅ is E or L; and X₆ is N, T, or S; and (ii) a sequence of LFACEVMHEALHNHYTYKL (SEQ ID NO:64).

In some embodiments, the modified CH3 domain comprises a sequence of AVEWFYDDSKLTN (SEQ ID NO:58), AVEWFYGNAKETN (SEQ ID NO:59), AVEWFYEAQKLNN (SEQ ID NO:60), AVEWFSEGSKETN (SEQ ID NO:61), AVEWFSGAQKESN (SEQ ID NO:62), or AVEWFSGAQKLTN (SEQ ID NO:63). In some embodiments, the modified CH3 domain comprises the sequence of LFACEVMHEALHNHYTYKL (SEQ ID NO:64).

A modified CH3 domain in the polypeotides described herein can comprise the sequence of AVEWFYDDSKLTN (SEQ ID NO:58) and the sequence of LFACEVMHEALHNHYTYKL (SEQ ID NO:64). A modified CH3 domain in the polypeotides described herein can comprise the sequence of AVEWFYGNAKETN (SEQ ID NO:59) and the sequence of LFACEVMHEALHNHYTYKL (SEQ ID NO:64). A modified CH3 domain in the polypeotides described herein can comprise the sequence of AVEWFYEAQKLNN (SEQ ID NO:60) and the sequence of LFACEVMHEALHNHYTYKL (SEQ ID NO:64). A modified CH3 domain in the polypeotides described herein can comprise the sequence of AVEWFSEGSKETN (SEQ ID NO:61) and the sequence of LFACEVMHEALHNHYTYKL (SEQ ID NO:64). A modified CH3 domain in the polypeotides described herein can comprise the sequence of AVEWFSGAQKESN (SEQ ID NO:62) and the sequence of LFACEVMHEALHNHYTYKL (SEQ ID NO:64). A modified CH3 domain in the polypeotides described herein can comprise the sequence of AVEWFSGAQKLTN (SEQ ID NO:63) and the sequence of LFACEVMHEALHNHYTYKL (SEQ ID NO:64).

In some embodiments of the polypeptide, the modified CH3 domain further comprises one, two, three, four, or five amino acid substitutions at positions comprising 419-421, 442, and 443, wherein the positions are determined according to EU numbering. In particular embodiments, the modified CH3 domain comprises Q or P at position 419, G or R at position 420, N or G at position 421, S or G at position 442, and/or L or E at position 443. In certain embodiments, the modified CH3 domain comprises P at position 419, R at position 420, G at position 421, G at position 442, and E at position 443.

The disclosure provides polypeptides comprising a sequence of

(SEQ ID NO: 68)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVX₁WFX₂X₃X₄X₅X₆X₇X₈NYKTTPPVLDSDGSFFLYSKLTVDKSRWQX₉
X₁₀X₁₁LFACEVMHEALX₁₂X₁₃HYTYKLLX₁₄X₁₅SPGK, wherein X₁
is E, N, F, or Y; X₂ is Y, S, A, or absent; X₃ is
G, D, E, or N; X₄ is D, G, N, or A; X₅ is Q, S,
G, A, or N; X₆ is K, I, R, or G; X₇ is E, L, D, or
Q; X₈ is N, T, S, or R; X₉ is Q or P; X₁₀ is G or
R; X₁₁ is N or G; X₁₂ is H or E; X₁₃ is N or G;
X₁₄ is S or G; and X₁₅ is L or E.

In some embodiments, the disclosure provides polypeptides comprising a sequence of:

(SEQ ID NO: 69)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVX₁WFX₂X₃X₄X₅X₆X₇X₈NYKTTPPVLDSDGSFFLYSKLTVDKSRWQX₉
X₁₀X₁₁LFACEVMHEALHNHYTYKLLX₁₂X₁₃SPGK, wherein X₁ is
E, N, F, or Y; X₂ is Y, S, A, or absent; X₃ is
G, D, E, or N; X₄ is D, G, N, or A; X₅ is Q, S,
G, A, or N; X₆ is K, I, R, or G; X₇ is E, L, D,
or Q; X₈ is N, T, S, or R; X₉ is Q or P; X₁₀ is
G or R; X₁₁ is N or G; X₁₂ is S or G; and X₁₃ is
L or E.

In some embodiments, the disclosure provides polypeptides comprising a sequence of:

(SEQ ID NO: 70)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVX₁WFX₂X₃X₄X₅X₆X₇X₈NYKTTPPVLDSDGSFFLYSKLTVDKSRWQQGN
LFACEVMHEALHNHYTYKLLSLSPGK, wherein X₁ is E, N,
F, or Y; X₂ is Y, S, A, or absent; X₃ is G, D,
E, or N; X₄ is D, G, N, or A; X₅ is Q, S, G, A,
or N; X₆ is K, I, R, or G; X₇ is E, L, D, or Q;
and X₈ is N, T, S, or R.

In some embodiments, the disclosure provides polypeptides comprising a sequence of:

(SEQ ID NO: 71)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVEWFX₁X₂X₃X₄KX₅X₆NYKTTPPVLDSDGSFFLYSKLTVDKSRWQQGNL
FACEVMHEALHNHYTYKLLSLSPGK, wherein X₁ is Y or S;
X₂ is G, D, or E; X₃ is D, G, N, or A; X₄ is Q,
S, or A; X₅ is E or L; and X₆ is N, T, or S.

In some embodiments, the modified CH3 domain comprises a sequence having at least 85% identity, at least 90% identity, or at least 95% identity (e.g., 95%, 96%, 97%, 98%, 99%, or 100% identity) to amino acids 111-217 of any one of SEQ ID NOS:72-77. In some embodiments, the modified CH3 domain comprises a sequence having at least 85% identity, at least 90% identity, or at least 95% identity (e.g., 95%, 96%, 97%, 98%, 99%, or 100% identity) to amino acids 111-217 of any one of SEQ ID NOS:72-77, in which amino acids at positions 380, 382-389, 422, 424, 426, 433, 434, 438, and/or 440, according to EU numbering, in each of SEQ ID NOS:72-77 are not changed. In certain embodiments, the modified CH3 domain comprises amino acids 111-217 of any one of SEQ ID NOS:72-77.

In some embodiments, the polypeptide (e.g., an Fc polypeptide) comprising a modified CH3 domain described herein comprises a sequence having at least 85% identity, at least 90% identity, or at least 95% identity (e.g., 95%, 96%, 97%, 98%, 99%, or 100% identity) to a sequence of any one of SEQ ID NOS:72-77. In some embodiments, the polypeptide (e.g., an Fc polypeptide) comprising a modified CH3 domain described herein comprises a sequence having at least 85% identity, at least 90% identity, or at least 95% identity (e.g., 95%, 96%, 97%, 98%, 99%, or 100% identity) to a sequence of any one of SEQ ID NOS:72-77, in which amino acids at positions 380, 382-389, 422, 424, 426, 433, 434, 438, and/or 440, according to EU numbering, in each of SEQ ID NOS:72-77 are not changed. In certain embodiments, the polypeptide (e.g., an Fc polypeptide) comprising a modified CH3 domain described herein comprises a sequence of any one of SEQ ID NOS:72-77.

A polypeptide (e.g., an Fc polypeptide) comprising a modified CH3 domain described herein can comprise: F at position 382, Y at position 383, D at position 384, D at position 385, S at position 386, K at position 387, L at position 388, T at position 389, P at position 419, R at position 420, G at position 421, L at position 422, A at position 424, E at position 426, Y at position 438, L at position 440, G at position 442, and E at position 443, wherein the positions are determined according to EU numbering.

A polypeptide (e.g., an Fc polypeptide) comprising a modified CH3 domain described herein can comprise: F at position 382, Y at position 383, G at position 384, N at position 385, A at position 386, K at position 387, T at position 389, L at position 422, A at position 424, E at position 426, Y at position 438, L at position 440, wherein the positions are determined according to EU numbering.

A polypeptide (e.g., an Fc polypeptide) comprising a modified CH3 domain described herein can comprise: F at position 382, Y at position 383, E at position 384, A at position 385, K at position 387, L at position 388, L at position 422, A at position 424, E at position 426, Y at position 438, L at position 440, wherein the positions are determined according to EU numbering.

A polypeptide (e.g., an Fc polypeptide) comprising a modified CH3 domain described herein can comprise: F at position 382, E at position 384, S at position 386, K at position 387, T at position 389, L at position 422, A at position 424, E at position 426, Y at position 438, L at position 440, wherein the positions are determined according to EU numbering.

A polypeptide (e.g., an Fc polypeptide) comprising a modified CH3 domain described herein can comprise: F at position 382, G at position 384, A at position 385, K at position 387, S at position 389, L at position 422, A at position 424, E at position 426, Y at position 438, L at position 440, wherein the positions are determined according to EU numbering.

A polypeptide (e.g., an Fc polypeptide) comprising a modified CH3 domain described herein can comprise: F at position 382, G at position 384, A at position 385, K at position 387, L at position 388, T at position 389, L at position 422, A at position 424, E at position 426, Y at position 438, L at position 440, wherein the positions are determined according to EU numbering.

In some embodiments, the polypeptides described above can further comprise W at position 366. In some embodiments, the polypeptides described above can further comprise S at position 366, A at position 368, and V at position 407. In certain embodiments, the polypeptides described above can further comprise A at position 234 and A at positon 235. In certain embodiments, the polypeptides described above can further comprise Gly or Ser at position 329. In some embodiments, the polypeptides described above can further comprise L at position 428 and S at position 434. The positions are determined according to EU numbering.

### VI. Additional Polypeptide Modifications

A polypeptide (e.g., an Fc polypeptide) comprising a modified CH3 domain as provided herein can also comprise additional mutations, e.g., to provide for knob and hole heterodimerization of the polypeptide, to modulate effector function, to extend serum half-life, to influence glyscosylation, and/or to reduce immunogenicity in humans.

### Polypeptide Modifications for Heterodimerization

In some embodiments, the polypeptides (e.g., an Fc polypeptide) comprising a modified CH3 domain described herein include mutations to promote heterodimer formation and hinder homodimer formation. These modifications are useful, for example, where it is desired to have only one of the polypeptide of a dimer have a CD98hc or TfR binding site (i.e., a monovalent CD98hc or TfR binder).

The knobs-into-holes approach generally involves introducing a protuberance (“knob”) at the interface of a polypeptide (e.g., an Fc polypeptide) and a corresponding cavity (“hole”) in the interface of a second polypeptide (e.g., an Fc polypeptide), such that the protuberance can be positioned in the cavity so as to promote heterodimer formation and thus hinder homodimer formation. Protuberances are constructed by replacing small amino acid side chains from the interface of the first polypeptide (e.g., an Fc polypeptide) with larger side chains (e.g., Tyr or Trp). Compensatory cavities of identical or similar size to the protuberances are created in the interface of the second polypeptide (e.g., an Fc polypeptide) by replacing large amino acid side chains with smaller ones (e.g., Ala or Thr). In some embodiments, such additional mutations are at a position in the polypeptide (e.g., an Fc polypeptide) that does not have a negative effect on binding of the polypeptide to CD98hc or TfR.

In one illustrative embodiment of a knob and hole approach for dimerization, position 366 of one of the polypeptides (e.g., an Fc polypeptide) comprises a Trp in place of a native Thr. The other polypeptide in the dimer has a Val at position 407 in place of the native Tyr. The other polypeptide (e.g., an Fc polypeptide) may further comprise a substitution in which the native Thr at position 366 is substituted with a Ser and a native Leu at position 368 is substituted with an Ala. Thus, one of the polypeptides (e.g., an Fc polypeptide) has the T366W knob mutation and the other polypeptide (e.g., an Fc polypeptide) has the Y407V hole mutation, which is typically accompanied by the T366S and L368A hole mutations. As indicated above, all positions are numbered per EU numbering.

In some embodiments, one or both polypeptides (e.g., Fc polypeptides) present in a polypeptide dimer (e.g., an Fc polypeptide dimer) can also be engineered to contain other modifications for heterodimerization, e.g., electrostatic engineering of contact residues within a CH3-CH3 interface that are naturally charged or hydrophobic patch modifications.

The knobs-into-holes approach (e.g., T366W knob substitution on one polypeptide (e.g., an Fc polypeptide) with the T366S, L368A, and Y407V hole substitution on the other polypeptide (e.g., an Fc polypeptide)) can be used with any of the polypeptides described herein (e.g., a CD98hc-binding polypeptide having a sequence of any one of SEQ ID NOS:28-45, or a TfR-binding polypeptide having a sequence of any one of SEQ ID NOS:72-77 or a sequence of any one of the clones listed in Table 29).

In some embodiments, only one of the two polypeptides (e.g., an Fc polypeptide) comprises a CD98hc-binding site (e.g., a CD98hc-binding polypeptide having a sequence of any one of SEQ ID NOS:28-45) while the other polypeptide (e.g., an Fc polypeptide) does not contain a CD98hc-binding site. In particular embodiments, one of the polypeptides (e.g., an Fc polypeptide) is a CD98hc-binding polypeptide and contains a knob mutation (e.g., T366W), while the other polypeptide (e.g., an Fc polypeptide) does not bind to CD98hc and contains a hole mutation (e.g., T366S, L368A, and Y407V). In other embodiments, one of the polypeptides (e.g., an Fc polypeptide) is a CD98hc-binding polypeptide and contains a hole mutation (e.g., T366S, L368A, and Y407V), while the other polypeptide (e.g., an Fc polypeptide) does not bind to CD98hc and contains a knob mutation (e.g., T366W).

In some embodiments, only one of the two polypeptides (e.g., an Fc polypeptide) comprises a TfR-binding site (e.g., a TfR-binding polypeptide having a sequence of any one of SEQ ID NOS:72-77 or a sequence of any one of the clones listed in Table 29) while the other polypeptide (e.g., an Fc polypeptide) does not contain a TfR-binding site. In particular embodiments, one of the polypeptides (e.g., an Fc polypeptide) is a TfR-binding polypeptide and contains a knob mutation (e.g., T366W), while the other polypeptide (e.g., an Fc polypeptide) does not bind to TfR and contains a hole mutation (e.g., T366S, L368A, and Y407V). In other embodiments, one of the polypeptides (e.g., an Fc polypeptide) is a TfR-binding polypeptide and contains a hole mutation (e.g., T366S, L368A, and Y407V), while the other polypeptide (e.g., an Fc polypeptide) does not bind to TfR and contains a knob mutation (e.g., T366W).

In particular embodiments, a polypeptide dimer (e.g., an Fc polypeptide dimer) that specifically binds to CD98hc can have a first polypeptide (e.g., an Fc polypeptide) having the T366W knob mutation and at least 85% (e.g., at least 86%, 87%, 88%, 89%, 90%, 91%, 92%, 93%, 94%, 95%, 96%, 97%, 98%, or 99%) or 100% identical to any one of SEQ ID NOS:28-45, and a second polypeptide (e.g., an Fc polypeptide) having the T366S, L368A, and Y407V hole mutations and at least 85% (e.g., at least 86%, 87%, 88%, 89%, 90%, 91%, 92%, 93%, 94%, 95%, 96%, 97%, 98%, or 99%) or 100% identical to SEQ ID NO:1. In other embodiments, a polypeptide dimer (e.g., an Fc polypeptide dimer) that specifically binds to CD98hc can have a first polypeptide (e.g., an Fc polypeptide) having the T366W knob mutation and at least 85% (e.g., at least 86%, 87%, 88%, 89%, 90%, 91%, 92%, 93%, 94%, 95%, 96%, 97%, 98%, or 99%) or 100% identical to SEQ ID NO:1, and a second polypeptide (e.g., an Fc polypeptide) having the T366S, L368A, and Y407V hole mutations and at least 85% (e.g., at least 86%, 87%, 88%, 89%, 90%, 91%, 92%, 93%, 94%, 95%, 96%, 97%, 98%, or 99%) or 100% identical to any one of SEQ ID NOS:28-45. In other embodiments, a polypeptide dimer (e.g., an Fc polypeptide dimer) that specifically binds to CD98hc can have a first polypeptide (e.g., an Fc polypeptide) having the T366W knob mutation and at least 85% (e.g., at least 86%, 87%, 88%, 89%, 90%, 91%, 92%, 93%, 94%, 95%, 96%, 97%, 98%, or 99%) or 100% identical to SEQ ID NOS:28-45, and a second polypeptide (e.g., an Fc polypeptide) having the T366S, L368A, and Y407V hole mutations and at least 85% (e.g., at least 86%, 87%, 88%, 89%, 90%, 91%, 92%, 93%, 94%, 95%, 96%, 97%, 98%, or 99%) or 100% identical to any one of SEQ ID NOS:28-45.

In particular embodiments, a polypeptide dimer (e.g., an Fc polypeptide dimer) that specifically binds to TfR can have a first polypeptide (e.g., an Fc polypeptide) having the T366W knob mutation and at least 85% (e.g., at least 86%, 87%, 88%, 89%, 90%, 91%, 92%, 93%, 94%, 95%, 96%, 97%, 98%, or 99%) or 100% identical to a sequence of any one of SEQ ID NOS:72-77 or a sequence of any one of the clones listed in Table 29, and a second polypeptide (e.g., an Fc polypeptide) having the T366S, L368A, and Y407V hole mutations and at least 85% (e.g., at least 86%, 87%, 88%, 89%, 90%, 91%, 92%, 93%, 94%, 95%, 96%, 97%, 98%, or 99%) or 100% identical to SEQ ID NO:1. In other embodiments, a polypeptide dimer (e.g., an Fc polypeptide dimer) that specifically binds to TfR can have a first polypeptide (e.g., an Fc polypeptide) having the T366W knob mutation and at least 85% (e.g., at least 86%, 87%, 88%, 89%, 90%, 91%, 92%, 93%, 94%, 95%, 96%, 97%, 98%, or 99%) or 100% identical to SEQ ID NO:1, and a second polypeptide (e.g., an Fc polypeptide) having the T366S, L368A, and Y407V hole mutations and at least 85% (e.g., at least 86%, 87%, 88%, 89%, 90%, 91%, 92%, 93%, 94%, 95%, 96%, 97%, 98%, or 99%) or 100% identical to a sequence of any one of SEQ ID NOS:72-77 or a sequence of any one of the clones listed in Table 29. In other embodiments, an Fc polypeptide dimer (e.g., an Fc polypeptide dimer) that specifically binds to TfR can have a first polypeptide (e.g., an Fc polypeptide) having the T366W knob mutation and at least 85% (e.g., at least 86%, 87%, 88%, 89%, 90%, 91%, 92%, 93%, 94%, 95%, 96%, 97%, 98%, or 99%) or 100% identical to a sequence of any one of SEQ ID NOS:72-77 or a sequence of any one of the clones listed in Table 29, and a second polypeptide (e.g., an Fc polypeptide) having the T366S, L368A, and Y407V hole mutations and at least 85% (e.g., at least 86%, 87%, 88%, 89%, 90%, 91%, 92%, 93%, 94%, 95%, 96%, 97%, 98%, or 99%) or 100% identical to a sequence of any one of SEQ ID NOS:72-77 or a sequence of any one of the clones listed in Table 29.

### Polypeptide Modifications for Modulating Effector Function

In some embodiments, a polypeptide dimer described herein is an Fc polypeptide dimer comprising two Fc polypeptides. In some embodiments, both Fc polypeptides in the Fc polypeptide dimer can comprise modifications that reduce or eliminate effector function, i.e., having a reduced ability to induce certain biological functions upon binding to an Fc receptor expressed on an effector cell that mediates the effector function. Effector cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells. Examples of antibody effector functions include, but are not limited to, Clq binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation.

Illustrative Fc polypeptide mutations that reduce effector function include, but are not limited to, substitutions in a CH2 domain, e.g., at positions 234 and 235 and/or at position 329, according to the EU numbering scheme. For example, in some embodiments, both Fc polypeptides comprise Ala residues at positions 234 and 235 (also referred to as “LALA” herein). In some embodiments, both Fc polypeptides comprise Gly residue at position 329 (also referred to as “P329G” or “PG” herein) or Ser residue at position 329 (also referred to as “P329S” or “PS” herein). In some embodiments, both Fc polypeptides comprise Ala residues at positions 234 and 235, and Gly residue at position 329 (also referred to as “LALA PG” herein). In some embodiments, both Fc polypeptides comprise Ala residues at positions 234 and 235, and Ser residue at position 329 (also referred to as “LALA PS” herein).

Additional Fc polypeptide mutations that modulate an effector function include, but are not limited to, the following: position 329 may have a mutation in which Pro is substituted with a Gly, Ala, Ser, or Arg or an amino acid residue large enough to destroy the Fc/Fc receptor interface that is formed between proline 329 of the Fc and Trp residues Trp87 and Trp110 of FcγRIII. Additional illustrative substitutions include S228P, E233P, L235E, N297A, N297D, and P331S, according to the EU numbering scheme. Multiple substitutions may also be present, e.g., L234A, L235A, and P329G of human IgG1; S228P and L235E of human IgG4; L234A and G237A of human IgG1; L234A, L235A, and G237A of human IgG1; V234A and G237A of human IgG2; L235A, G237A, and E318A of human IgG4; and S228P and L236E of human IgG4, according to the EU numbering scheme.

In some embodiments, a polypeptide (e.g., an Fc polypeptide) that specifically binds to CD98hc comprises LALA substitutions and a sequence having at least 85% (e.g., at least 86%, 87%, 88%, 89%, 90%, 91%, 92%, 93%, 94%, 95%, 96%, 97%, 98%, or 99%) or 100% identity to any one of SEQ ID NOS:28-45.

In some embodiments, a polypeptide (e.g., an Fc polypeptide) that specifically binds to CD98hc comprises LALA and P329G or P329S substitutions and a sequence having at least 85% (e.g., at least 86%, 87%, 88%, 89%, 90%, 91%, 92%, 93%, 94%, 95%, 96%, 97%, 98%, or 99%) or 100% identity to any one of SEQ ID NOS:28-45.

In some embodiments, a polypeptide (e.g., an Fc polypeptide) that specifically binds to TfR comprises LALA substitutions and a sequence having at least 85% (e.g., at least 86%, 87%, 88%, 89%, 90%, 91%, 92%, 93%, 94%, 95%, 96%, 97%, 98%, or 99%) or 100% identity to a sequence of any one of SEQ ID NOS:72-77 or a sequence of any one of the clones listed in Table 29.

In some embodiments, a polypeptide (e.g., an Fc polypeptide) that specifically binds to TfR comprises LALA and P329G or P329S substitutions and a sequence having at least 85% (e.g., at least 86%, 87%, 88%, 89%, 90%, 91%, 92%, 93%, 94%, 95%, 96%, 97%, 98%, or 99%) or 100% identity to a sequence of any one of SEQ ID NOS:72-77 or a sequence of any one of the clones listed in Table 29.

### Polypeptide Modifications for Extending Serum Half-Life

In some embodiments, modifications to enhance serum half-life can be introduced into any polypeptides described herein. For example, in some embodiments, a polypeptide dimer described herein is an Fc polypeptide dimer comprising two Fc polypeptides. In some embodiments, both Fc polypeptides in the Fc polypeptide dimer can comprise M428L and N434S substitutions (also referred to as LS substitutions), as numbered according to the EU numbering scheme. Alternatively, both Fc polypeptides in an Fc polypeptide dimer can have an N434S or N434A substitution. Alternatively, both Fc polypeptides in an Fc polypeptide dimer can have an M428L substitution. In other embodiments, both Fc polypeptides in an Fc polypeptide dimer can comprise M252Y, S254T, and T256E substitutions.

In any of the embodiments described herein, a polypeptide (e.g., an Fc polypeptide) that specifically binds to CD98hc can further comprise LS substitutions. For example, in some embodiments, a polypeptide (e.g., an Fc polypeptide) that specifically binds to CD98hc comprises LS substitutions and a sequence having at least 85% (e.g., at least 86%, 87%, 88%, 89%, 90%, 91%, 92%, 93%, 94%, 95%, 96%, 97%, 98%, or 99%) or 100% identity to any one of SEQ ID NOS:28-45.

In any of the embodiments described herein, a polypeptide (e.g., an Fc polypeptide) that specifically binds to TfR can further comprise LS substitutions. For example, in some embodiments, a polypeptide (e.g., an Fc polypeptide) that specifically binds to TfR comprises LS substitutions and a sequence having at least 85% (e.g., at least 86%, 87%, 88%, 89%, 90%, 91%, 92%, 93%, 94%, 95%, 96%, 97%, 98%, or 99%) or 100% identity to a sequence of any one of SEQ ID NOS:72-77 or a sequence of any one of the clones listed in Table 29.

Polypeptide with C-terminal Lysine Residue Removed

In some embodiments, one or both of the polypeptides (e.g., Fc polypeptides) can have its C-terminal lysine removed (e.g., the Lys residue at position 447 of the Fc polypeptide, according to EU numbering). The C-terminal lysine residue is highly conserved in immunoglobulins across many species and may be fully or partially removed by the cellular machinery during protein production. In some embodiments, removal of the C-terminal lysines in the Fc polypeptides can improve the stability of the proteins.

### VII. Illustrative Polypeptides that Bind to CD98HC

A modified CH3 domain of the present disclosure may be joined to a CH2 domain, which may be a naturally occurring CH2 domain or a variant CH2 domain, typically at the C-terminal end of the CH2 domain to form a polypeptide (e.g., an Fc polypeptide) that binds to CD98hc. In some embodiments, the polypeptide (e.g., an Fc polypeptide) further comprises a partial or full hinge region of an antibody, which is joined to the N-terminal end of the CH2 domain. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:4).

In certain embodiments, provided herein are CD98hc-binding polypeptides which when bound to human CD98hc, binds to at least 1, 2, 3, 4, 5, 6, 7, 8, 9, 10, 11, 12, 13 or 14 of the residues selected from positions of the group consisting of: 477, 478, 479, 480, 481, 482, 483, 486, 499, 497, 498, 500, 501, and 502 of SEQ ID NO: 134. In certain embodiments, provided herein are CD98hc-binding polypeptides which when bound to human CD98hc, binds to at least 1, 2, 3, 4, 5, 6, 7, 8, 9, 10, 11, 12, 13 or 14 of the residues selected from positions of the group consisting of: 477, 478, 479, 480, 481, 482, 483, 486, 499, 497, 498, 500, 501, and 502 of SEQ ID NO: 134, and optionally binds additionally to at least 1, 2, 3, 4, 5, 6, 7, or 8 additional residue selected from positions of the group consisting of: 229, 231, 232, 236, 235, 488, 495, and 496 of SEQ ID NO: 134 or at least 1, 2, 3, 4, 5, 6, 7, 8, 9, 10, 11, or 12 additional residue selected from positions of the group consisting of: 312, 315, 348, 381, 439, 444, 443, 485, 484, 476, 475, and 442 of SEQ ID NO: 134. In some embodiments, provided herein are CD98hc-binding polypeptides which, when bound to human CD98hc, the polypeptide binds to positions 477, 478, 479, 480, 481, 482, 483, 486, 499, 497, 498, 500, 501, and 502 of SEQ ID NO: 134. In certain embodiments, provided herein are CD98hc-binding polypeptides which when bound to human CD98hc, binds to at least 1, 2, 3, 4, 5, 6, 7, 8, 9, 10, 11, 12, 13, 14, 15, 16, 17, 18, 19, 20, 21 or 22 of the residues selected from positions of the group consisting of: 229, 231, 232, 236, 235, 486, 488, 495, 496, 498, 500, 499, 497, 482, 481, 483, 477, 480, 501, 502, 478, and 479 of SEQ ID NO:134. In some embodiments, provided herein are CD98hc-binding polypeptides which when bound to human CD98hc, binds the residues at positions 229, 231, 232, 236, 235, 486, 488, 495, 496, 498, 500, 499, 497, 482, 481, 483, 477, 480, 501, 502, 478, and 479 of SEQ ID NO:134. In certain embodiments, provided herein are CD98hc-binding polypeptides which when bound to human CD98hc, binds to at least 1, 2, 3, 4, 5, 6, 7, 8, 9, 10, 11, 12, 13, 14, 15, 16, 17, 18, 19, 20, 21, 22, 23, 24, 25, or 26 of the residues selected from positions of the group consisting of: 312, 315, 348, 381, 439, 444, 443, 485, 484, 477, 483, 481, 480, 478, 476, 502, 499, 501, 500, 498, 497, 486, 479, 482, 475, and 442 of SEQ ID NO: 134. In some embodiments, provided herein are CD98hc-binding polypeptides which when bound to human CD98hc, binds to the residues at positions 312, 315, 348, 381, 439, 444, 443, 485, 484, 477, 483, 481, 480, 478, 476, 502, 499, 501, 500, 498, 497, 486, 479, 482, 475, and 442 of SEQ ID NO: 134.

In some embodiments, a polypeptide (e.g., an Fc polypeptide) can comprise a sequence from Table 2A and the polypeptide (e.g., an Fc polypeptide) can be further modified to contain a CD98hc binding site in the modified CH3 domain as described herein.

TABLE 2A
Fc Sequences For Further CD98hc- or TfR Binding Site Modifications

| | | | | | | LALA- | LALA- |
|---|---|---|---|---|---|---|---|
| | LALA | LALA-PG | LALA-PS | LS | LALA-LS | PG-LS | PS-LS |
| WT Fc | SEQ ID | SEQ ID | SEQ ID | SEQ ID | SEQ ID | SEQ ID | SEQ ID |
| (SEQ ID | NO: 7 | NO: 10 | NO: 13 | NO: 16 | NO: 19 | NO: 22 | NO: 25 |
| NO: 1) | | | | | | | |
| Fc w/knob | SEQ ID | SEQ ID | SEQ ID | SEQ ID | SEQ ID | SEQ ID | SEQ ID |
| (SEQ ID | NO: 8 | NO: 11 | NO: 14 | NO: 17 | NO: 20 | NO: 23 | NO: 26 |
| NO: 5) | | | | | | | |
| Fc w/hole | SEQ ID | SEQ ID | SEQ ID | SEQ ID | SEQ ID | SEQ ID | SEQ ID |
| (SEQ ID | NO: 9 | NO: 12 | NO: 15 | NO: 18 | NO: 21 | NO: 24 | NO: 27 |
| NO: 6) | | | | | | | |

In further embodiments, the polypeptide (e.g., an Fc polypeptide) described herein can be further joined to another moiety, for example, a Fab fragment, thus generating a CD98hc-binding Fc-Fab fusion. In some embodiments, the CD98hc-binding Fc-Fab fusion comprises a modified CH3 domain, a CH2 domain, a hinge region, and a Fab fragment. The Fab fragment may be to any target of interest, e.g., a therapeutic neurological target, where the Fab is delivered to the target by transcytosis across the BBB mediated by the binding of the modified CH3 domain polypeptide to CD98hc.

The CD98hc-binding polypeptide (e.g., a CD98hc-binding Fc polypeptide) may also be fused to a polypeptide of interest other than a Fab. For example, in some embodiments, the CD98hc-binding polypeptide (e.g., a CD98hc-binding Fc polypeptide) may be fused to a polypeptide that is desirable to target to a CD98hc-expressing cell or to deliver across an endothelium, e.g., the BBB, by transcytosis. In some embodiments, the CD98hc-binding polypeptide (e.g., a CD98hc-binding Fc polypeptide) is fused to a soluble protein. In still other embodiments, the CD98hc-binding polypeptide (e.g., a CD98hc-binding Fc polypeptide) may be fused to a peptide or protein useful in protein purification, e.g., polyhistidine, epitope tags, e.g., FLAG, c-Myc, hemagglutinin tags and the like, glutathione S transferase (GST), thioredoxin, protein A, protein G, or maltose binding protein (MBP). In some cases, the peptide or protein to which the CD98hc-binding polypeptide (e.g., a CD98hc-binding Fc polypeptide) is fused may comprise a protease cleavage site, such as a cleavage site for Factor Xa or Thrombin.

### CD98hc-Binding Polypeptides

### LLB2

In some embodiments, the polypeptides that bind to CD98hc are from the LLB2 family. In some embodiments, the polypeptide comprises at least eleven, twelve, thirteen, fourteen, or fifteen substitutions in a set of amino acid positions consisting of 378, 380, 382, 383, 384, 385, 386, 387, 389, 391, 421, 422, 424, 426, 428, 434, 436, 438, 440, 441, and 442.

In some embodiments, the substitutions are selected from a S, V, D, E, or Y at position 378, a L, I, M, A, Q, V, or K at position 380, a N, S, L, M, P, Y, K, A, or T at position 382, a T, F, N, P, D, L, H, or Q at position 383, a K, R, H, I, L, F, Y, V, or Q at position 384, a F or Y at position 385, a V, L, A, I, F, Y, S, T, H, R, or E at position 386, a L or I at position 387, a D, Q, A, T, H, or V at position 389, a T, V, or A at position 391, a E, Q, or A at position 421, a L, M, I, T, or P at position 422, an A at position 424, a N at position 426, a L, T, P, Y F, I, A, K, H, or W at position 428, a S at position 434, a L, V, H, F, P, R or W at position 436, a F or W at position 438, a L, P, E, N, V, A, I, or D at position 440, a P at position 441, and an A, V, M, Q, F, P, L, Y, K, R, H, or M at position 442. In some embodiments, the polypeptide comprises a sequence of any one of SEQ ID NOS:5-27 and at least eleven, twelve, thirteen, fourteen, or fifteen substitutions in a set of amino acid positions consisting of a S, V, D, E, or Y at position 378, a L, I, M, A, Q, V, or K at position 380, a N, S, L, M, P, Y, K, A, or T at position 382, a T, F, N, P, D, L, H, or Q at position 383, a K, R, H, I, L, F, Y, V, or Q at position 384, a F or Y at position 385, a V, L, A, I, F, Y, S, T, H, R, or E at position 386, a L or I at position 387, a D, Q, A, T, H, or V at position 389, a T, V, or A at position 391, a E, Q, or A at position 421, a L, M, I, T, or P at position 422, an A at position 424, a N at position 426, a L, T, P, Y F, I, A, K, H, or W at position 428, a S at position 434, a L, V, H, F, P, R or W at position 436, a F or W at position 438, a L, P, E, N, V, A, I, or D at position 440, a P at position 441, and an A, V, M, Q, F, P, L, Y, K, R, H, or M at position 442.

In some embodiments, the polypeptide comprises a modified constant domain (e.g., a modified CH3 domain) that comprises a sequence having at least 85%, 90%, or 95% sequence identity to amino acids 111-217 of the sequence of SEQ ID NOS:28-43. In some embodiments, the polypeptide comprises a modified constant domain (e.g., a modified CH3 domain) that comprises a sequence having at least 85%, 90%, or 95% sequence identity to amino acids 111-217 of the sequence of SEQ ID NOS:28-43, wherein the modified constant domain comprises at least eleven, twelve, thirteen, fourteen, or fifteen substitutions in a set of amino acid positions consisting of a S, V, D, E, or Y at position 378, a L, I, M, A, Q, V, or K at position 380, a N, S, L, M, P, Y, K, A, or T at position 382, a T, F, N, P, D, L, H, or Q at position 383, a K, R, H, I, L, F, Y, V, or Q at position 384, a F or Y at position 385, a V, L, A, I, F, Y, S, T, H, R, or E at position 386, a L or I at position 387, a D, Q, A, T, H, or V at position 389, a T, V, or A at position 391, a E, Q, or A at position 421, a L, M, I, T, or P at position 422, an A at position 424, a N at position 426, a L, T, P, Y F, I, A, K, H, or W at position 428, a S at position 434, a L, V, H, F, P, R or W at position 436, a F or W at position 438, a L, P, E, N, V, A, I, or D at position 440, a P at position 441, and an A, V, M, Q, F, P, L, Y, K, R, H, or M at position 442.

In some embodiments, the polypeptide comprises at least eleven, twelve, thirteen, fourteen, or fifteen substitutions in a set of amino acid positions consisting of 380, 382, 384, 385, 386, 387, 421, 422, 424, 426, 428, 436, 438, 440, and 442. In some embodiments, the substitutions are selected from a L at position 380, a N at position 382, a R, H, or Q at position 384, a F or Y at position 385, a V, L, I, F, Y, or E at position 386, a L at position 387, a E, Q or A at position 421, a I, T, or P at position 422, an A at position 424, a N at position 426, a Y or W at position 428, a R or W at position 436, a F or W at position 438, a N at position 440, and an A, Q, K, R, H, or M at position 442. In some embodiments, the polypeptide comprises a sequence of any one of SEQ ID NOS:5-27 and at least eleven, twelve, thirteen, fourteen, or fifteen substitutions in a set of amino acid positions consisting of a L at position 380, a N at position 382, a R, H, or Q at position 384, a F or Y at position 385, a V, L, I, F, Y, or E at position 386, a L at position 387, a E, Q, or A at position 421, a I, T, or P at position 422, an A at position 424, a N at position 426, a Y or W at position 428, a R or W at position 436, a F or W at position 438, a N at position 440, and an A, Q, K, R, H, or M at position 442.

In some embodiments, the polypeptide comprises a modified constant domain (e.g., a modified CH3 domain) that comprises a sequence having at least 85%, 90%, or 95% sequence identity to amino acids 111-217 of the sequence of SEQ ID NOS:28-43, wherein the modified constant domain comprises at least eleven, twelve, thirteen, fourteen, or fifteen substitutions in a set of amino acid positions consisting of a L at position 380, a N at position 382, a R, H, or Q at position 384, a F or Y at position 385, a V, L, I, F, Y, or E at position 386, a L at position 387, a E, Q, or A at position 421, a I, T, or P at position 422, an A at position 424, a N at position 426, a Y or W at position 428, a R or W at position 436, a F or W at position 438, a N at position 440, and an A, Q, K, R, H, or M at position 442.

In some embodiments, a polypeptide (e.g., an Fc polypeptide) that specifically binds to a CD98hc comprises a modified CH3 domain, wherein the modified CH3 domain comprises: (i) a first amino acid sequence of LX₁NX₂X₃X₄X₅L (SEQ ID NO:46), wherein X₁ is any amino acid, wherein X₂ is R, H, or Q, wherein X₃ is F or Y, wherein X₄ is V, L, I, F, Y, or E, wherein X₅ is any amino acid; (ii) a second amino acid sequence of X₁X₂X₃AX₄X₅X₆X₇ (SEQ ID NO:47), wherein X₁ is E, N, Q, or A, wherein X₂ is I, V, T, or P, wherein X₃ and X₄ are any amino acid, wherein X₅ is N or S, wherein X₆ is any amino acid, wherein X₇ is Y or W; and (iii) a third amino acid sequence of X₁X₂X₃X₄NX₅X₆ (SEQ ID NO:48), wherein X₁ is Y, R, or W, wherein X₂ is any amino acid, wherein X₃ is F or W, wherein X₄ and X₅ are any amino acid, and wherein X₆ is A, Q, K, R, H, M, or S.

### LLB2-10⁻⁶

In certain embodiments, a polypeptide comprises SEQ ID NO:28. In one embodiment, a monovalent dimer (e.g., a monovalent Fc dimer) comprises a polypeptide (e.g., an Fc polypeptide) having a L at position 380, a N at position 382, a R at position 384, a F at position 385, a V at position 386, a L at position 387, an I at position 422, an A at position 424, a N at position 426, a Y at position 428, a F at position 438, a N at position 440, and an A at position 442. In one embodiment, the polypeptide (e.g., an Fc polypeptide) further comprises a T366W knob mutation.

### LLB2-10⁻⁸

In certain embodiments, a polypeptide comprises SEQ ID NO:29. In one embodiment, a monovalent dimer (e.g., a monovalent Fc dimer) comprises a polypeptide (e.g., an Fc polypeptide) having a L at position 380, a N at position 382, a R at position 384, a F at position 385, a V at position 386, a L at position 387, an E at position 421, an I at position 422, an A at position 424, a N at position 426, a Y at position 428, a F at position 438, a N at position 440, and an A at position 442. In another embodiment, a bivalent dimer (e.g., a bivalent Fc dimer) comprises two polypeptides (e.g., Fc polypeptides) each having a L at position 380, a N at position 382, a R at position 384, a F at position 385, a V at position 386, a L at position 387, an E at position 421, an I at position 422, an A at position 424, a N at position 426, a Y at position 428, a F at position 438, a N at position 440, and an A at position 442. In one embodiment, the polypeptide (e.g., an Fc polypeptide) further comprises a T366W knob mutation.

### LLB2-10⁻⁸-d18

In certain embodiments, a polypeptide comprises SEQ ID NO:30. In one embodiment, a bivalent dimer (e.g., a bivalent Fe dimer) comprises two polypeptides (e.g., Fc polypeptides) each having a L at position 380, a N at position 382, a Q at position 384, a Y at position 385, a E at position 386, a L at position 387, an A at position 424, a N at position 426, a Y at position 428, a F at position 438, a N at position 440, and an A at position 442.

### LLB2-10⁻⁸-d12

In certain embodiments, a polypeptide comprises SEQ ID NO:31. In one embodiment, a bivalent dimer (e.g., a bivalent Fc dimer) comprises two polypeptides (e.g., Fc polypeptides) each having a L at position 380, a N at position 382, a H at position 384, a Y at position 385, a E at position 386, a L at position 387, an A at position 424, a N at position 426, a Y at position 428, a F at position 438, a N at position 440, and an A at position 442.

### LLB2-10⁻⁸-d6

In certain embodiments, a polypeptide comprises SEQ ID NO:32. In one embodiment, a monovalent dimer (e.g., a monovalent Fc dimer) comprises a polypeptide (e.g., an Fc polypeptide) having a L at position 380, a N at position 382, a R at position 384, a F at position 385, a V at position 386, a L at position 387, an A at position 424, a N at position 426, a Y at position 428, a F at position 438, a N at position 440, and an A at position 442. In another embodiment, a bivalent dimer (e.g., a bivalent Fc dimer) comprises two polypeptides (e.g., Fc polypeptides) each having a L at position 380, a N at position 382, a R at position 384, a F at position 385, a V at position 386, a L at position 387, an A at position 424, a N at position 426, a Y at position 428, a F at position 438, a N at position 440, and an A at position 442. In one embodiment, the polypeptide (e.g., an Fc polypeptide) further comprises a T366W knob mutation.

### LLB2-10⁻⁸-d3

In certain embodiments, a polypeptide comprises SEQ ID NO:33. In one embodiment, a monovalent dimer (e.g., a monovalent Fc dimer) comprises a polypeptide (e.g., an Fc polypeptide) having a L at position 380, a N at position 382, a R at position 384, a F at position 385, a V at position 386, a L at position 387, an E at position 421, an A at position 424, a N at position 426, a Y at position 428, a F at position 438, a N at position 440, and an A at position 442. In another embodiment, a bivalent dimer (e.g., a bivalent Fc dimer) comprises two polypeptides (e.g., Fc polypeptides) each having a L at position 380, a N at position 382, aR at position 384, aF at position 385, a V at position 386, a L at position 387, an E at position 421, an A at position 424, a N at position 426, a Y at position 428, a F at position 438, a N at position 440, and an A at position 442. In one embodiment, the polypeptide (e.g., an Fc polypeptide) further comprises a T366W knob mutation.

### LLB2-10⁻⁸-d1

In certain embodiments, a polypeptide comprises SEQ ID NO:34. In one embodiment, a bivalent dimer (e.g., a bivalent Fe dimer) comprises two polypeptides (e.g., Fc polypeptides) each having a L at position 380, a N at position 382, a R at position 384, a F at position 385, a V at position 386, a L at position 387, an E at position 421, an I at position 422, an A at position 424, a N at position 426, a Y at position 428, a F at position 438, and a N at position 440.

### LLB2.10.8.10.3

In certain embodiments, a polypeptide comprises SEQ ID NO:35. In one embodiment, a monovalent dimer (e.g., a monovalent Fe dimer) comprises a polypeptide (e.g., an Fc polypeptide) having a L at position 380, a N at position 382, a R at position 384, a F at position 385, a V at position 386, a L at position 387, an I at position 422, an A at position 424, a N at position 426, a Y at position 428, a F at position 438, a N at position 440, and a R at position 442. In one embodiment, the polypeptide (e.g., an Fc polypeptide) further comprises a T366W knob mutation.

### LLB2.10.8.10.8

In certain embodiments, a polypeptide comprises SEQ ID NO:36. In one embodiment, a monovalent dimer (e.g., a monovalent Fe dimer) comprises a polypeptide (e.g., an Fc polypeptide) having a L at position 380, a N at position 382, a R at position 384, a F at position 385, a V at position 386, a L at position 387, an I at position 422, an A at position 424, a N at position 426, a Y at position 428, a F at position 438, a N at position 440, and a H at position 442. In one embodiment, the polypeptide (e.g., an Fc polypeptide) further comprises a T366W knob mutation.

### LLB2.10.8.14.3

In certain embodiments, a polypeptide comprises SEQ ID NO:37. In one embodiment, a monovalent dimer (e.g., a monovalent Fe dimer) comprises a polypeptide (e.g., an Fc polypeptide) having a L at position 380, a N at position 382, a R at position 384, a F at position 385, a V at position 386, a L at position 387, an I at position 422, an A at position 424, a N at position 426, a Y at position 428, a Rat position 436, aF at position 438, aN at position 440, and a R at position 442. In one embodiment, the polypeptide (e.g., an Fc polypeptide) further comprises a T366W knob mutation.

### LLB2.10.8.12.5

In certain embodiments, a polypeptide comprises SEQ ID NO:38. In one embodiment, a monovalent dimer (e.g., a monovalent Fe dimer) comprises a polypeptide (e.g., an Fc polypeptide) having a L at position 380, a N at position 382, a H at position 384, a Y at position 385, a E at position 386, a L at position 387, an I at position 422, an A at position 424, a N at position 426, a Y at position 428, a F at position 438, a N at position 440, and an A at position 442. In one embodiment, the polypeptide (e.g., an Fc polypeptide) further comprises a T366W knob mutation.

### LLB2-37

In certain embodiments, a polypeptide comprises SEQ ID NO:39. In one embodiment, a monovalent dimer (e.g., a monovalent Fe dimer) comprises a polypeptide (e.g., an Fc polypeptide) having a L at position 380, a N at position 382, a Q at position 384, a F at position 385, a H at position 386, a L at position 387, an I at position 422, an A at position 424, a N at position 426, a Y at position 428, a F at position 438, a N at position 440, and a L at position 442. In another embodiment, a bivalent dimer (e.g., a bivalent Fe dimer) comprises two polypeptides (e.g., Fc polypeptides) each having a L at position 380, a N at position 382, a Q at position 384, a F at position 385, a H at position 386, a L at position 387, an I at position 422, an A at position 424, a N at position 426, a Y at position 428, a F at position 438, a N at position 440, and a L at position 442. In one embodiment, the polypeptide (e.g., an Fc polypeptide) further comprises a T366W knob mutation.

### LLB2.10.8.9.11.N

In certain embodiments, a polypeptide comprises SEQ ID NO:40. In one embodiment, a monovalent dimer (e.g., a monovalent Fe dimer) comprises a polypeptide (e.g., an Fc polypeptide) having a L at position 380, a N at position 382, a R at position 384, a F at position 385, a V at position 386, a L at position 387, an T at position 422, an A at position 424, a N at position 426, a Y at position 428, a F at position 438, a N at position 440, and an A at position 442.

### LLB2.10.8.10.1

In certain embodiments, a polypeptide comprises SEQ ID NO:41. In one embodiment, a monovalent dimer (e.g., a monovalent Fe dimer) comprises a polypeptide (e.g., an Fc polypeptide) having a L at position 380, a N at position 382, a R at position 384, a F at position 385, a V at position 386, a L at position 387, an I at position 422, an A at position 424, a N at position 426, a Y at position 428, a F at position 438, a N at position 440, and a K at position 442. In one embodiment, the polypeptide (e.g., an Fc polypeptide) further comprises a T366W knob mutation.

### LLB2.10.8.4.12

In certain embodiments, a polypeptide comprises SEQ ID NO:42. In one embodiment, a monovalent dimer (e.g., a monovalent Fe dimer) comprises a polypeptide (e.g., an Fc polypeptide) having a L at position 380, a N at position 382, a R at position 384, a F at position 385, a V at position 386, a L at position 387, an I at position 422, an A at position 424, a N at position 426, a Y at position 428, a W at position 436, a F at position 438, a N at position 440, and a R at position 442. In one embodiment, the polypeptide (e.g., an Fc polypeptide) further comprises a T366W knob mutation.

### LLB2.10.8.2.1

In certain embodiments, a polypeptide comprises SEQ ID NO:43. In one embodiment, a monovalent dimer (e.g., a monovalent Fe dimer) comprises a polypeptide (e.g., an Fc polypeptide) having a L at position 380, a N at position 382, a Q at position 384, a Y at position 385, a L at position 386, a L at position 387, an E at position 421, an I at position 422, an A at position 424, a N at position 426, a Y at position 428, a F at position 438, a N at position 440, and an A at position 442.

Additional polypeptides (e.g., Fc polypeptides) from the LLB2 family that specifically bind to CD98hc are shown in Tables A2-A8 and A12.

TABLE 2B
Illustrative CD98hc binding site modifications

| Name | Set of modifications |
|---|---|
| LLB2-10-6 | a L at position 380, a N at position 382, a R at position 384, a F at |
| | position 385, a V at position 386, a L at position 387, an I at position |
| | 422, an A at position 424, a N at position 426, a Y at position 428, a F |
| | at position 438, a N at position 440, and an A at position 442 |
| LLB2-10-8 | a L at position 380, a N at position 382, a R at position 384, a F at |
| | position 385, a V at position 386, a L at position 387, an E at position |
| | 421, an I at position 422, an A at position 424, a N at position 426, a Y |
| | at position 428, a F at position 438, a N at position 440, and an A at |
| | position 442 |
| LLB2-10-8-d18 | a L at position 380, a N at position 382, a Q at position 384, a Y at |
| | position 385, a E at position 386, a L at position 387, an A at position |
| | 424, a N at position 426, a Y at position 428, a F at position 438, a N |
| | at position 440, and an A at position 442 |
| LLB2-10-8-d12 | a L at position 380, a N at position 382, a H at position 384, a Y at |
| | position 385, a E at position 386, a L at position 387, an A at position |
| | 424, a N at position 426, a Y at position 428, a F at position 438, a N |
| | at position 440, and an A at position 442 |
| LLB2-10-8-d6 | a L at position 380, a N at position 382, a R at position 384, a F at |
| | position 385, a V at position 386, a L at position 387, an A at position |
| | 424, a N at position 426, a Y at position 428, a F at position 438, a N |
| | at position 440, and an A at position 442 |
| LLB2-10-8-d3 | a L at position 380, a N at position 382, a R at position 384, a F at |
| | position 385, a V at position 386, a L at position 387, an E at position |
| | 421, an A at position 424, a N at position 426, a Y at position 428, a F |
| | at position 438, a N at position 440, and an A at position 442 |
| LLB2-10-8-d1 | a L at position 380, a N at position 382, a R at position 384, a F at |
| | position 385, a V at position 386, a L at position 387, an E at position |
| | 421, an I at position 422, an A at position 424, a N at position 426, a Y |
| | at position 428, a F at position 438, and a N at position 440 |
| LLB2.10.8.10.3 | a L at position 380, a N at position 382, a R at position 384, a F at |
| | position 385, a V at position 386, a L at position 387, an I at position |
| | 422, an A at position 424, a N at position 426, a Y at position 428, a F |
| | at position 438, a N at position 440, and a R at position 442 |
| LLB2.10.8.10.8 | a L at position 380, a N at position 382, a R at position 384, a F at |
| | position 385, a V at position 386, a L at position 387, an I at position |
| | 422, an A at position 424, a N at position 426, a Y at position 428, a F |
| | at position 438, a N at position 440, and a H at position 442 |
| LLB2.10.8.14.3 | a L at position 380, a N at position 382, a R at position 384, a F at |
| | p10.8.12.5osition 385, a V at position 386, a L at position 387, an I at |
| | position 422, an A at position 424, a N at position 426, a Y at position |
| | 428, a R at position 436, a F at position 438, a N at position 440, and a |
| | R at position 442 |
| LLB2.10.8.12.5 | a L at position 380, a N at position 382, a H at position 384, a Y at |
| | position 385, a E at position 386, a L at position 387, an I at position |
| | 422, an A at position 424, a N at position 426, a Y at position 428, a F |
| | at position 438, a N at position 440, and an A at position 442 |
| LLB2-37 | a L at position 380, a N at position 382, a Q at position 384, a F at |
| | position 385, a H at position 386, a L at position 387, an I at position |
| | 422, an A at position 424, a N at position 426, a Y at position 428, a F |
| | at position 438, a N at position 440, and a L at position 442 |
| LLB2.10.8.9.11.N | a L at position 380, a N at position 382, a R at position 384, a F at |
| | position 385, a V at position 386, a L at position 387, an T at position |
| | 422, an A at position 424, a N at position 426, a Y at position 428, a F |
| | at position 438, a N at position 440, and an A at position 442 |
| LLB2.10.8.10.1 | a L at position 380, a N at position 382, a R at position 384, a F at |
| | position 385, a V at position 386, a L at position 387, an I at position |
| | 422, an A at position 424, a N at position 426, a Y at position 428, a F |
| | at position 438, a N at position 440, and a K at position 442 |
| LLB2.10.8.4.12 | a L at position 380, a N at position 382, a R at position 384, a F at |
| | position 385, a V at position 386, a L at position 387, an I at position |
| | 422, an A at position 424, a N at position 426, a Y at position 428, a |
| | W at position 436, a F at position 438, a N at position 440, and a R at |
| | position 442 |
| LLB2.10.8.2.1 | a L at position 380, a N at position 382, a Q at position 384, a Y at |
| | position 385, a L at position 386, a L at position 387, an E at position |
| | 421, an I at position 422, an A at position 424, a N at position 426, a Y |
| | at position 428, a F at position 438, a N at position 440, and an A at |
| | position 442 |

### LLB1

In some embodiments, the polypeptides (e.g., Fc polypeptides) that bind to CD98hc are from the LLB1 family. In some embodiments, the polypeptide (e.g., an Fc polypeptide) comprises at least eight, nine, eleven, twelve, thirteen, fourteen, fifteen, sixteen, seventeen, eighteen, or nineteen substitutions in a set of amino acid positions consisting of 378, 380, 382, 383, 384, 385, 386, 387, 389, 421, 422, 424, 426, 428, 434, 436, 438, 440, and 442. In some embodiments, the substitutions are selected from a S or V at position 378, a D, M, N, P, F, or H at position 380, a R, Y, F, S, W, Y, K, or N at position 382, a T at position 383, a L, Y, A, S, or F at position 384, a F, K, D, M, I, N, Y, L, or H at position 385, a T, P, E, K, A, V, D, T, or F at position 386, a N, L, Y, R, F, G, S, D, or T at position 387, a T, Y, or F at position 389, a D, E, or Q at position 421, a I, K, L, R, T, F, or H at position 422, a V, W, G, L, I, P, or Y at position 424, a D, A, Q, W, L, or P at position 426, a L or Y at position 428, a S at position 434, a F at position, 436, a I, V, F, N, P, or S at position 438, and a K, T, P, I, or F at position 440, and a Q or M at position 442. In some embodiments, the polypeptide (e.g., an Fc polypeptide) comprises a sequence of any one of SEQ ID NOS:5-27 and at least eight, nine, ten, eleven, twelve, thirteen, fourteen, fifteen, sixteen, seventeen, eighteen, or nineteen substitutions in a set of amino acid positions consisting of a S or V at position 378, a D, M, N, P, F, or H at position 380, a R, Y, F, S, W, Y, K, or N at position 382, a T at position 383, a L, Y, A, S, or F at position 384, a F, K, D, M, I, N, Y, L, or H at position 385, a T, P, E, K, A, V, D, T, or F at position 386, a N, L, Y, R, F, G, S, D, or T at position 387, a T, Y, or F at position 389, a D, E, or Q at position 421, a I, K, L, R, T, F, or H at position 422, a V, W, G, L, I, P, or Y at position 424, a D, A, Q, W, L, or P at position 426, a L or Y at position 428, a S at position 434, a F at position, 436, a I, V, F, N, P, or S at position 438, and a K, T, P, I, or F at position 440, and a Q or M at position 442.

In some embodiments, the polypeptide (e.g., an Fc polypeptide) comprises a sequence having at least 85%, 90%, or 95% sequence identity to amino acids 111-217 of the sequence of SEQ ID NOS:44-45. In some embodiments, the polypeptide (e.g., an Fc polypeptide) comprises a modified constant domain (e.g., a modified CH3 domain) that comprises a sequence having at least 85%, 90%, or 95% sequence identity to amino acids 111-217 of the sequence of SEQ ID NOS:44-45, wherein the modified constant domain comprises at least eight, nine, ten, eleven, twelve, thirteen, fourteen, fifteen, sixteen, seventeen, eighteen, or nineteen substitutions in a set of amino acid positions consisting of a S or V at position 378, a D, M, N, P, F, or H at position 380, a R, Y, F, S, W, Y, K, or N at position 382, a T at position 383, a L, Y, A, S, or F at position 384, a F, K, D, M, I, N, Y, L, or H at position 385, a T, P, E, K, A, V, D, T, or F at position 386, a N, L, Y, R, F, G, S, D, or T at position 387, a T, Y, or F at position 389, a D, E, or Q at position 421, a I, K, L, R, T, F, or H at position 422, a V, W, G, L, I, P, or Y at position 424, a D, A, Q, W, L, or P at position 426, a L or Y at position 428, a S at position 434, a F at position, 436, a I, V, F, N, P, or S at position 438, and a K, T, P, I, or F at position 440, and a Q or M at position 442.

In some embodiments, the polypeptides (e.g., Fc polypeptides) that bind to CD98hc are from the LLB1 family. In some embodiments, the polypeptide (e.g., an Fc polypeptide) comprises at least eight, nine, ten, eleven, twelve, or thirteen substitutions in a set of amino acid positions consisting of 380, 382, 384, 385, 386, 387, 422, 424, 426, 428, 434, 438, and 440. In some embodiments, the substitutions are selected from a D, M, N, P, F, or H at position 380, a R, Y, F, S, W, Y, K, or N at position 382, a L, Y, A, S, or F at position 384, a F, K, D, M, I, N, Y, L, or H at position 385, a T, P, E, K, A, V, D, T, or F at position 386, a N, L, Y, R, G, S, D, or T at position 387, a I, K, R, T, F, or H at position 422, a V, W, G, L, I, P, or Y at position 424, a D, A, Q, W, L, or P at position 426, a L at position 428, a S at position 434, a I, F, N, P, or S at position 438, and a K, T, I, or F at position 440. In some embodiments, the polypeptide (e.g., an Fc polypeptide) comprises a sequence of any one of SEQ ID NOS:5-27 and at least eight, nine, ten, eleven, twelve, or thirteen substitutions in a set of amino acid positions consisting of a D, M, N, P, F, or H at position 380, a R, Y, F, S, W, Y, K, or N at position 382, a L, Y, A, S, or F at position 384, a F, K, D, M, I, N, Y, L, or H at position 385, a T, P, E, K, A, V, D, T, or F at position 386, a N, L, Y, R, G, S, D, or T at position 387, a I, K, R, T, F, or H at position 422, a V, W, G, L, I, P, or Y at position 424, a D, A, Q, W, L, or P at position 426, a L at position 428, a S at position 434, a I, F, N, P, or S at position 438, and a K, T, I, or F at position 440.

In some embodiments, the polypeptide (e.g., an Fc polypeptide) comprises a modified constant domain (e.g., a modified CH3 domain) that comprises a sequence having at least 85%, 90%, or 95% sequence identity to amino acids 111-217 of the sequence of SEQ ID NOS:44-45, wherein the modified constant domain comprises at least eight, nine, ten, eleven, twelve, or thirteen substitutions in a set of amino acid positions consisting of a D, M, N, P, F, or H at position 380, a R, Y, F, S, W, Y, K, or N at position 382, a L, Y, A, S, or F at position 384, a F, K, D, M, I, N, Y, L, or H at position 385, a T, P, E, K, A, V, D, T, or F at position 386, a N, L, Y, R, G, S, D, or T at position 387, a I, K, R, T, F, or H at position 422, a V, W, G, L, I, P, or Y at position 424, a D, A, Q, W, L, or P at position 426, a L at position 428, a S at position 434, a I, F, N, P, or S at position 438, and a K, T, I, or F at position 440.

In some embodiments, a polypeptide (e.g., an Fc polypeptide) comprises at least eight, nine, ten, eleven, twelve, thirteen, fourteen, fifteen, sixteen, seventeen, eighteen, or nineteen substitutions in a set of amino acid positions consisting of 378, 380, 382, 383, 384, 385, 386, 387, 389, 421, 422, 424, 426, 428, 434, 436, 438, 440, and 442. In some embodiments, the substitutions are selected from a S or V at position 378, a D at position 380, a R at position 382, a T at position 383, a Y at position 384, a K at position 385, a P at position 386, a Y at position 387, a T, Y, or F at position 389, a D, E, or Q at position 421, an I at position 422, a V at position 424, a D at position 426, a L or Y at position 428, a S at position 434, a F at position 436, an I or V at position 438, a K at position 440, and a Q or M at position 442. In some embodiments, the polypeptide (e.g., an Fc polypeptide) comprise a sequence of any one of SEQ ID NOS:5-27 and at least eight, nine, ten, eleven, twelve, thirteen, fourteen, fifteen, sixteen, seventeen, eighteen, or nineteen in a set of amino acid positions consisting of a S or V at position 378, a D at position 380, a R at position 382, a T at position 383, a Y at position 384, a K at position 385, alP at position 386, a Y at position 387, a T, Y, or F at position 389, a D, E, or Q at position 421, an I at position 422, a V at position 424, a D at position 426, a L or Y at position 428, a S at position 434, a F at position 436, an I or V at position 438, a K at position 440, and a Q or M at position 442.

In some embodiments, the polypeptide (e.g., an Fc polypeptide) comprises a modified constant domain (e.g., a modified CH3 domain) that comprises a sequence having at least 85%, 90%, or 95% sequence identity to amino acids 111-217 of the sequence of SEQ ID NOS:44-45, wherein the modified constant domain comprises at least eight, nine, ten, eleven, twelve, thirteen, fourteen, fifteen, sixteen, seventeen, eighteen, or nineteen in a set of amino acid positions consisting of a S or V at position 378, a D at position 380, a R at position 382, a T at position 383, a Y at position 384, a K at position 385, a P at position 386, a Y at position 387, a T, Y, or F at position 389, a D, E, or Q at position 421, an I at position 422, a V at position 424, a D at position 426, a L or Y at position 428, a S at position 434, a F at position 436, an I or V at position 438, a K at position 440, and a Q or M at position 442.

In some embodiments, a polypeptide (e.g., an Fc polypeptide) comprises at least eight, nine, ten, eleven, twelve, thirteen, fourteen, or fifteen substitutions in a set of amino acid positions consisting of 382, 383, 384, 385, 386, 387, 389, 421, 422, 424, 426, 428, 436, 438, and 440. In some embodiments, the substitutions are selected from a R at position 382, a T at position 383, a Y at position 384, a K at position 385, a P at position 386, a Y at position 387, a T at position 389, a D at position 421, an I at position 422, a V at position 424, a D at position 426, a L at position 428, a F at position 436, a I at position 438, and a K at position 440. In some embodiments, the polypeptide (e.g., an Fc polypeptide) comprises a sequence of any one of SEQ ID NOS:5-27 and at least eight, nine, ten, eleven, twelve, thirteen, fourteen, or fifteen in a set of amino acid positions consisting of a R at position 382, a T at position 383, a Y at position 384, a K at position 385, a P at position 386, a Y at position 387, a T at position 389, a D at position 421, an I at position 422, a V at position 424, a D at position 426, a L at position 428, a F at position 436, a I at position 438, and a K at position 440.

In some embodiments, the polypeptide (e.g., an Fc polypeptide) comprises a modified constant domain (e.g., a modified CH3 domain) that comprises a sequence having at least 85%, 90%, or 95% sequence identity to amino acids 111-217 of the sequence of SEQ ID NO:28-43, wherein the modified constant domain comprises at least eight, nine, ten, eleven, twelve, thirteen, fourteen, or fifteen in a set of amino acid positions consisting of a R at position 382, a T at position 383, a Y at position 384, a K at position 385, a P at position 386, a Y at position 387, a T at position 389, a D at position 421, an I at position 422, a V at position 424, a D at position 426, a L at position 428, a F at position 436, a I at position 438, and a K at position 440.

In some embodiments, a polypeptide (e.g., an Fc polypeptide) that specifically binds to a CD98hc comprises a modified CH3 domain, wherein the modified CH3 domain comprises: (i) a first amino acid sequence of X₁X₂YKPYX₃T (SEQ ID NO:49), wherein X₁ is E or R, wherein X₂ is S or T, wherein X₃ is any amino acid; (ii) a second amino acid sequence of X₁X₂X₃VX₄DX₅X₆ (SEQ ID NO:50), wherein X₁ is N or D, wherein X₂ is V or I, wherein X₃, X₄, and X₅ and are any amino acid, wherein X₆ is M or L; and (iii) a third amino acid sequence of X₁X₂IX₃X₄ (SEQ ID NO:51), wherein X₁ is Y or F, wherein X₂ and X₃ are any amino acid, wherein and X₄ is S or K.

### LLB1-3-16-2

In certain embodiments, a polypeptide comprises SEQ ID NO:44. In one embodiment, a monovalent dimer (e.g., a monovalent Fe dimer) comprises a polypeptide (e.g., an Fc polypeptide) having a R at position 382, a T at position 383, a Y at position 384, a K at position 385, a P at position 386, a Y at position 387, a T at position 389, a D at position 421, an I at position 422, a V at position 424, a D at position 426, a F at position 436, a I at position 438, and a K at position 440. In one embodiment, the polypeptide (e.g., an Fc polypeptide) further comprises a T366W knob mutation.

### LLB1-3-16

In certain embodiments, a polypeptide comprises SEQ ID NO:45. In one embodiment, a monovalent dimer (e.g., a monovalent Fe dimer) comprises a polypeptide (e.g., an Fc polypeptide) having a R at position 382, a T at position 383, a Y at position 384, a K at position 385, a P at position 386, a Y at position 387, a T at position 389, a D at position 421, an I at position 422, a V at position 424, a D at position 426, a L at position 428, a F at position 436, a I at position 438, and a K at position 440. In one embodiment, the polypeptide (e.g., an Fc polypeptide) further comprises a T366W knob mutation.

Additional polypeptides (e.g., Fc polypeptides) from the LLB1 family that specifically bind to CD98hc are shown in Tables A9-A11 and A13.

### VIII. Illustrative Polypeptides that Bind to TfR

A CH3 domain of the present disclosure may be joined to a CH2 domain, which may be a naturally occurring CH2 domain or a variant CH2 domain, typically at the C-terminal end of the CH2 domain to form a polypeptide (e.g., an Fc polypeptide) that binds to TfR. In some embodiments, the polypeptide (e.g., an Fc polypeptide) further comprises a partial or full hinge region of an antibody, which is joined to the N-terminal end of the CH2 domain. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:4).

In some embodiments, a polypeptide (e.g., an Fc polypeptide) can comprise a sequence from Table 2A and the polypeptide (e.g., an Fc polypeptide) can be further modified to contain a TfR binding site in the modified CH3 domain as described herein.

In further embodiments, the polypeptide (e.g., an Fc polypeptide) can be further joined to another moiety, for example, a Fab fragment, thus generating a TfR-binding Fc-Fab fusion. In some embodiments, the TfR-binding Fc-Fab fusion comprises a modified CH3 domain, a CH2 domain, a hinge region, and a Fab fragment. The Fab fragment may be to any target of interest, e.g., a therapeutic neurological target, where the Fab is delivered to the target by transcytosis across the BBB mediated by the binding of the modified CH3 domain polypeptide to TfR.

The TfR-binding polypeptide (e.g., a TfR-binding Fc polypeptide) may also be fused to a polypeptide of interest other than a Fab. For example, in some embodiments, the TfR-binding polypeptide (e.g., a TfR-binding Fc polypeptide) may be fused to a polypeptide that is desirable to target to a TfR-expressing cell or to deliver across an endothelium, e.g., the BBB, by transcytosis. In some embodiments, the TfR-binding polypeptide (e.g., a TfR-binding Fc polypeptide) is fused to a soluble protein. In still other embodiments, the TfR-binding polypeptide (e.g., a TfR-binding Fc polypeptide) may be fused to a peptide or protein useful in protein purification, e.g., polyhistidine, epitope tags, e.g., FLAG, c-Myc, hemagglutinin tags and the like, glutathione S transferase (GST), thioredoxin, protein A, protein G, or maltose binding protein (MBP). In some cases, the peptide or protein to which the TfR-binding polypeptide (e.g., a TfR-binding Fc polypeptide) is fused may comprise a protease cleavage site, such as a cleavage site for Factor Xa or Thrombin.

### TfR-Binding Polypeptides

42.2.19

In certain embodiments, a polypeptide comprises the sequence of SEQ ID NO:72. Further, a polypeptide can comprise a sequence of any one of SEQ ID NOS:78, 84, 90, 96, 102, 108, 114, and 120. In one embodiment, a monovalent dimer (e.g., a monovalent Fc dimer) comprises a polypeptide (e.g., an Fc polypeptide) having F at position 382, Y at position 383, D at position 384, D at position 385, S at position 386, K at position 387, L at position 388, T at position 389, P at position 419, R at position 420, G at position 421, L at position 422, A at position 424, E at position 426, Y at position 438, L at position 440, G at position 442, and E at position 443, wherein the positions are determined according to EU numbering. In one embodiment, the polypeptide (e.g., an Fc polypeptide) in the monovalent dimer (e.g., a monovalent Fc dimer) further comprises a T366W knob mutation. In another embodiment, the polypeptide (e.g., an Fc polypeptide) in the monovalent dimer (e.g., a monovalent Fc dimer) further comprises T366S, L368A, and Y407V hole mutations. In another embodiment, a bivalent dimer (e.g., a bivalent Fe dimer) comprises two polypeptides (e.g., Fc polypeptides) each having F at position 382, Y at position 383, D at position 384, D at position 385, S at position 386, K at position 387, L at position 388, T at position 389, P at position 419, R at position 420, G at position 421, L at position 422, A at position 424, E at position 426, Y at position 438, L at position 440, G at position 442, and E at position 443, wherein the positions are determined according to EU numbering.

### 42.2.3-1H

In certain embodiments, a polypeptide comprises the sequence of SEQ ID NO:73. Further, a polypeptide can comprise a sequence of any one of SEQ ID NOS:79, 85, 91, 97, 103, 109, 115, and 121. In one embodiment, a monovalent dimer (e.g., a monovalent Fc dimer) comprises a polypeptide (e.g., an Fc polypeptide) having F at position 382, Y at position 383, G at position 384, N at position 385, A at position 386, K at position 387, T at position 389, L at position 422, A at position 424, E at position 426, Y at position 438, L at position 440, wherein the positions are determined according to EU numbering. In one embodiment, the polypeptide (e.g., an Fc polypeptide) in the monovalent dimer (e.g., a monovalent Fc dimer) further comprises a T366W knob mutation. In another embodiment, the polypeptide (e.g., an Fc polypeptide) in the monovalent dimer (e.g., a monovalent Fc dimer) further comprises T366S, L368A, and Y407V hole mutations. In another embodiment, a bivalent dimer (e.g., a bivalent Fc dimer) comprises two polypeptides (e.g., Fc polypeptides) each having F at position 382, Y at position 383, G at position 384, N at position 385, A at position 386, K at position 387, T at position 389, L at position 422, A at position 424, E at position 426, Y at position 438, L at position 440, wherein the positions are determined according to EU numbering.

42.8.196

In certain embodiments, a polypeptide comprises the sequence of SEQ ID NO:74. Further, a polypeptide can comprise a sequence of any one of SEQ ID NOS:80, 86, 92, 98, 104, 110, 116, and 122. In one embodiment, a monovalent dimer (e.g., a monovalent Fc dimer) comprises a polypeptide (e.g., an Fc polypeptide) having F at position 382, Y at position 383, E at position 384, A at position 385, K at position 387, L at position 388, L at position 422, A at position 424, E at position 426, Y at position 438, L at position 440, wherein the positions are determined according to EU numbering. In one embodiment, the polypeptide (e.g., an Fc polypeptide) in the monovalent dimer (e.g., a monovalent Fc dimer) further comprises a T366W knob mutation. In another embodiment, the polypeptide (e.g., an Fc polypeptide) in the monovalent dimer (e.g., a monovalent Fe dimer) further comprises T366S, L368A, and Y407V hole mutations. In another embodiment, a bivalent dimer (e.g., a bivalent Fe dimer) comprises two polypeptides (e.g., Fc polypeptides) each having F at position 382, Y at position 383, E at position 384, A at position 385, K at position 387, L at position 388, L at position 422, A at position 424, E at position 426, Y at position 438, L at position 440, wherein the positions are determined according to EU numbering.

42.8.80

In certain embodiments, a polypeptide comprises the sequence of SEQ ID NO:75. Further, a polypeptide can comprise a sequence of any one of SEQ ID NOS:81, 87, 93, 99, 105, 111, 117, and 123. In one embodiment, a monovalent dimer (e.g., a monovalent Fe dimer) comprises a polypeptide (e.g., an Fc polypeptide) having F at position 382, E at position 384, S at position 386, K at position 387, T at position 389, L at position 422, A at position 424, E at position 426, Y at position 438, L at position 440, wherein the positions are determined according to EU numbering. In one embodiment, the polypeptide (e.g., an Fc polypeptide) in the monovalent dimer (e.g., a monovalent Fe dimer) further comprises a T366W knob mutation. In another embodiment, the polypeptide (e.g., an Fc polypeptide) in the monovalent dimer (e.g., a monovalent Fe dimer) further comprises T366S, L368A, and Y407V hole mutations. In another embodiment, a bivalent dimer (e.g., a bivalent Fe dimer) comprises two polypeptides (e.g., Fc polypeptides) each having F at position 382, E at position 384, S at position 386, K at position 387, T at position 389, L at position 422, A at position 424, E at position 426, Y at position 438, L at position 440, wherein the positions are determined according to EU numbering.

42.8.15

In certain embodiments, a polypeptide comprises the sequence of SEQ ID NO:76. Further, a polypeptide can comprise a sequence of any one of SEQ ID NOS:82, 88, 94, 100, 106, 112, 118, and 124. In one embodiment, a monovalent dimer (e.g., a monovalent Fe dimer) comprises a polypeptide (e.g., an Fc polypeptide) having F at position 382, G at position 384, A at position 385, K at position 387, S at position 389, L at position 422, A at position 424, E at position 426, Y at position 438, L at position 440, wherein the positions are determined according to EU numbering. In one embodiment, the polypeptide (e.g., an Fc polypeptide) in the monovalent dimer (e.g., a monovalent Fe dimer) further comprises a T366W knob mutation. In another embodiment, the polypeptide (e.g., an Fc polypeptide) in the monovalent dimer (e.g., a monovalent Fe dimer) further comprises T366S, L368A, and Y407V hole mutations. In another embodiment, a bivalent dimer (e.g., a bivalent Fe dimer) comprises two polypeptides (e.g., Fc polypeptides) each having F at position 382, G at position 384, A at position 385, K at position 387, S at position 389, L at position 422, A at position 424, E at position 426, Y at position 438, L at position 440, wherein the positions are determined according to EU numbering.

42.8.17

In certain embodiments, a polypeptide comprises the sequence of SEQ ID NO:77. Further, a polypeptide can comprise a sequence of any one of SEQ ID NOS:83, 89, 95, 101, 107, 113, 119, and 125. In one embodiment, a monovalent dimer (e.g., a monovalent Fc dimer) comprises a polypeptide (e.g., an Fc polypeptide) having F at position 382, G at position 384, A at position 385, K at position 387, L at position 388, T at position 389, L at position 422, A at position 424, E at position 426, Y at position 438, L at position 440, wherein the positions are determined according to EU numbering. In one embodiment, the polypeptide (e.g., an Fc polypeptide) in the monovalent dimer (e.g., a monovalent Fc dimer) further comprises a T366W knob mutation. In another embodiment, the polypeptide (e.g., an Fc polypeptide) in the monovalent dimer (e.g., a monovalent Fc dimer) further comprises T366S, L368A, and Y407V hole mutations. In another embodiment, a bivalent dimer (e.g., a bivalent Fc dimer) comprises two polypeptides (e.g., Fc polypeptides) each having F at position 382, G at position 384, A at position 385, K at position 387, L at position 388, T at position 389, L at position 422, A at position 424, E at position 426, Y at position 438, L at position 440, wherein the positions are determined according to EU numbering.

### IX. Dimers for CD98HC-Binding Site Modification

In some embodiments, a polypeptide (e.g., an Fc polypeptide) that binds to CD98hc can form a dimer (e.g., an Fc dimer) comprising two polypeptides (e.g., Fc polypeptides). The dimer may be a heterodimer or a homodimer.

### Dimer That Binds CD98hc Bivalently

In some embodiments, the dimer is an Fc dimer that comprises two Fc polypeptides in which each contains a CD98hc binding site, i.e., binds CD98hc bivalently. In an Fc dimer that binds CD98hc bivalently, the first and second Fc polypeptides may comprise the same modified CH3 domain. In other embodiments, the second Fc polypeptide may comprise a different modified CH3 domain from that in the first Fc polypeptide to provide a second CD98hc-binding site.

In some embodiments, a bivalent Fe dimer that specifically binds to CD98hc described herein comprises a first and second Fc polypeptide pair from Table 2C and (i) each of the first and second Fc polypeptides are further modified to contain a CD98hc binding site in the modified CH3 domain as described herein or (ii) wherein the first and second Fc polypeptides contain a CD98hc binding site in the modified CH3 domain as described herein. In other embodiments, a bivalent Fc dimer that specifically binds to CD98hc described herein comprises a first and second Fc polypeptide pair from Table 2C, wherein the first polypeptide has at least 85% (e.g., at least 86%, 87%, 88%, 89%, 90%, 91%, 92%, 93%, 94%, 95%, 96%, 97%, 98%, or 99%) or 100% identity to the sequence from first Fc polypeptide sequence from Table 2C, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2C, and wherein each of the first and second Fc polypeptides are further modified to contain a CD98hc binding site in the modified CH3 domain as described herein. In one embodiment, a bivalent Fc dimer that specifically binds to CD98hc described herein comprises a first and second Fc polypeptide pair from Table 2C, wherein the first polypeptide has at least 85% (e.g., at least 86%, 87%, 88%, 89%, 90%, 91%, 92%, 93%, 94%, 95%, 96%, 97%, 98%, or 99%) or 100% identity to the sequence from first Fc polypeptide sequence from Table 2C, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polylpeptide sequence from Table 2C, and wherein each of the first and second Fc polypeptides are further modified to contain a CD98hc binding site in the modified CH3 domain comprising: (i) at least eleven, twelve, thirteen, fourteen, or fifteen substitutions in a set of amino acid positions consisting of 380, 382, 384, 385, 386, 387, 421, 422, 424, 426, 428, 436, 438, 440, and 442. In some embodiments, the substitutions are selected from a L at position 380, a N at position 382, a R, H, or Q at position 384, a F or Y at position 385, a V, L, I, F, Y, or E at position 386, a L at position 387, a E, Q, or A at position 421, a I, T, or P at position 422, an A at position 424, a N at position 426, a Y or W at position 428, a R or W at position 436, a F or W at position 438, a N at position 440, and an A, Q, K, R, H, or M at position 442, (ii) at least eleven, twelve, thirteen, fourteen, or fifteen substitutions in a set of amino acid positions consisting of a S, V, D, E, or Y at position 378, a L, I, M, A, Q, V, or K at position 380, a N, S, L, M, P, Y, K, A, or T at position 382, a T, F, N, P, D, L, H, or Q at position 383, a K, R, H, I, L, F, Y, V, or Q at position 384, a F or Y at position 385, a V, L, A, I, F, Y, S, T, H, R, or E at position 386, a L or I at position 387, a D, Q, A, T, H, or V at position 389, a T, V, or A at position 391, a E, Q, or A at position 421, a L, M, I, T, orP at position 422, an A at position 424, a N at position 426, a L, T, P, Y F, I, A, K, H, or W at position 428, a S at position 434, a L, V, H, F, P, R or W at position 436, a F or W at position 438, a L, P, E, N, V, A, I, or D at position 440, a P at position 441, and an A, V, M, Q, F, P, L, Y, K, R, H, or M at position 442, or (iii) at least eight, nine, ten, eleven, twelve, thirteen, fourteen, fifteen, sixteen, seventeen, eighteen, or nineteen substitutions in a set of amino acid positions consisting of a S or V at position 378, a D, M, N, P, F, or H at position 380, a R, Y, F, S, W, Y, K, or N at position 382, a T at position 383, a L, Y, A, S, or F at position 384, a F, K, D, M, I, N, Y, L, or H at position 385, a T, P, E, K, A, V, D, T, or F at position 386, a N, L, Y, R, F, G, S, D, or T at position 387, a T, Y, or F at position 389, a D, E, or Q at position 421, a I, K, L, R, T, F, or H at position 422, a V, W, G, L, I, P, or Y at position 424, a D, A, Q, W, L, or P at position 426, a L or Y at position 428, a S at position 434, a F at position, 436, a I, V, F, N, P, or S at position 438, and a K, T, P, I, or F at position 440, and a Q or M at position 442.

In one embodiment, a bivalent Fe dimer that specifically binds to CD98hc described herein comprises a first and second Fc polypeptide pair from Table 2C, wherein the first polypeptide has at least 85% (e.g., at least 86%, 87%, 88%, 89%, 90%, 91%, 92%, 93%, 94%, 95%, 96%, 97%, 98%, or 99%) or 100% identity to the sequence from first Fc polypeptide sequence from Table 2C, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2C, and wherein each of the first and second Fc polypeptides are further modified to contain a CD98hc binding site in the modified CH3 domain comprising a set of modifications selected from Table 2B.

### Dimer That Binds CD98hc Monovalently

In some embodiments, the dimer is a monovalent Fc dimer that comprises two Fc polypeptides, in which only one of the two Fc polypeptides in the monovalent Fc dimer comprises a CD98hc-binding site, while the other Fc polypeptide does not bind to CD98hc. In addition, the Fc polypeptides can contain modifications for promoting heterodimzerization of the Fc dimer (e.g., T366W; and T366S, L368A, and Y407V). In some embodiments, a monovalent Fc dimer that specifically binds to CD98hc described herein comprises a first and second Fc polypeptide pair from Table 2D and the first Fc polypeptide is further modified to contain a CD98hc binding site in the modified CH3 domain as described herein. In other embodiments, a monovalent Fe dimer that specifically binds to CD98hc described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the sequence from first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the first Fc polypeptides is further modified to contain a CD98hc binding site in the modified CH3 domain as described herein. In one embodiment, a monovalent Fc dimer that specifically binds to CD98hc described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the sequence from first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the first Fc polypeptide is further modified to contain a CD98hc binding site in the modified CH3 domain comprising: (i) at least eleven, twelve, thirteen, fourteen, or fifteen substitutions in a set of amino acid positions consisting of 380, 382, 384, 385, 386, 387, 421, 422, 424, 426, 428, 436, 438, 440, and 442. In some embodiments, the substitutions are selected from a L at position 380, a N at position 382, a R, H, or Q at position 384, a F or Y at position 385, a V, L, I, F, Y, or E at position 386, a L at position 387, a E, Q, or A at position 421, a I, T, or P at position 422, an A at position 424, a N at position 426, a Y or W at position 428, a R or W at position 436, a F or W at position 438, a N at position 440, and an A, Q, K, R, H, or M at position 442, (ii) at least eleven, twelve, thirteen, fourteen, or fifteen substitutions in a set of amino acid positions consisting of a S, V, D, E, or Y at position 378, a L, I, M, A, Q, V, or K at position 380, a N, S, L, M, P, Y, K, A, or T at position 382, a T, F, N, P, D, L, H, or Q at position 383, a K, R, H, I, L, F, Y, V, or Q at position 384, a F or Y at position 385, a V, L, A, I, F, Y, S, T, H, R, or E at position 386, a L or I at position 387, a D, Q, A, T, H, or V at position 389, a T, V, or A at position 391, a E, Q, or A at position 421, a L, M, I, T, or P at position 422, an A at position 424, a N at position 426, a L, T, P, Y F, I, A, K, H, or W at position 428, a S at position 434, a L, V, H, F, P, R or W at position 436, a F or W at position 438, a L, P, E, N, V, A, I, or D at position 440, a P at position 441, and an A, V, M, Q, F, P, L, Y, K, R, H, or M at position 442, or (iii), at least eight, nine, ten, eleven, twelve, thirteen, fourteen, fifteen, sixteen, seventeen, eighteen, or nineteen substitutions in a set of amino acid positions consisting of a S or V at position 378, a D, M, N, P, F, or H at position 380, a R, Y, F, S, W, Y, K, or N at position 382, a T at position 383, a L, Y, A, S, or F at position 384, a F, K, D, M, I, N, Y, L, or H at position 385, a T, P, E, K, A, V, D, T, or F at position 386, a N, L, Y, R, F, G, S, D, or T at position 387, a T, Y, or F at position 389, a D, E, or Q at position 421, a I, K, L, R, T, F, or H at position 422, a V, W, G, L, I, P, or Y at position 424, a D, A, Q, W, L, or P at position 426, a L or Y at position 428, a S at position 434, a F at position, 436, a I, V, F, N, P, or S at position 438, and a K, T, P, I, or F at position 440, and a Q or M at position 442.

For example, the first Fc polypeptide from dimer pair K from Table 2D (i.e., SEQ ID NO:11) is further modified to comprise a L at position 380, a N at position 382, a R at position 384, a F at position 385, a V at position 386, a L at position 387, an E at position 421, an I at position 422, an A at position 424, a N at position 426, a Y at position 428, a F at position 438, a N at position 440, and an A at position 442.

In one embodiment, a monovalent Fe dimer that specifically binds to CD98hc described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the sequence from first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2B, and wherein the first Fc polypeptide is further modified to contain a CD98hc binding site in the modified CH3 domain comprising a set of modifications selected from Table 2B.

### X. Dimers for Tfr-Binding Site Modification

In some embodiments, a polypeptide (e.g., an Fc polypeptide) that binds to TfR can form a dimer (e.g., an Fc dimer) comprising two polypeptides (e.g., Fc polypeptides). The dimer may be a heterodimer or a homodimer.

### Dimer That Binds TfR Bivalently

In some embodiments, the dimer is an Fc dimer that comprises two polypeptides (e.g., Fc polypeptides) in which each contains a TfR binding site, i.e., binds TfR bivalently. In an Fc dimer that binds TfR bivalently, the first and second Fc polypeptides may comprise the same CH3 domain. In other embodiments, the second Fc polypeptide may comprise a different CH3 domain from that in the first Fc polypeptide to provide a second TfR-binding site.

In some embodiments, a bivalent Fe dimer that specifically binds to TfR described herein comprises a first and second Fc polypeptide pair from Table 2C and (i) each of the first and second Fc polypeptides are further modified to contain a CD98hc binding site in the modified CH3 domain as described herein or (ii) wherein the first and second Fc polypeptides contain a CD98hc binding site in the modified CH3 domain as described herein. In other embodiments, a bivalent Fc dimer that specifically binds to TfR described herein comprises a first and second Fc polypeptide pair from Table 2C, wherein the first Fc polypeptide has at least 85% (e.g., at least 86%, 87%, 88%, 89%, 90%, 91%, 92%, 93%, 94%, 95%, 96%, 97%, 98%, or 99%) or 100% identity to the first Fc polypeptide sequence from Table 2C, wherein the second Fc polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2C, and wherein each of the first and second Fc polypeptides are further modified to contain a TfR binding site in the modified CH3 domain as described herein.

In one embodiment, a bivalent Fc dimer that specifically binds to TfR described herein comprises a first and second Fc polypeptide pair from Table 2C, wherein the first Fc polypeptide has at least 85% (e.g., at least 86%, 87%, 88%, 89%, 90%, 91%, 92%, 93%, 94%, 95%, 96%, 97%, 98%, or 99%) or 100% identity to the first Fc polypeptide sequence from Table 2C, wherein the second Fc polypeptide has at least 85% (e.g., at least 86%, 87%, 88%, 89%, 90%, 91%, 92%, 93%, 94%, 95%, 96%, 97%, 98%, or 99%) or 100% identity to the second Fc polylpeptide sequence from Table 2C, and wherein each of the first and second Fc polypeptides are further modified to contain a TfR binding site in the modified CH3 domain comprising: three, four, five, six, seven, or eight amino acid substitutions and/or one or two amino acid deletions in a set of amino acid positions comprising 380 and 382-389; and five, six, or seven amino acid substitutions in a set of amino acid positions comprising 422, 424, 426, 433, 434, 438, and 440, wherein the positions are determined according to EU numbering.

In some embodiments, the substitutions and/or deletions are selected from: E, N, F, or Y at position 380, F at position 382, Y, S, A, or an amino acid deletion at position 383, G, D, E, or N at position 384, D, G, N, or A at position 385, Q, S, G, A, or N at position 386, K, I, R, or G at position 387, E, L, D, or Q at position 388, N, T, S, or R at position 389, L at position 422, A at position 424, E at position 426, H or E at position 433, N or G at position 434, Y at position 438, and L at position 440.

In one embodiment, a bivalent Fe dimer that specifically binds to TfR described herein comprises a first and second Fc polypeptide pair from Table 2C, wherein the first Fc polypeptide has at least 85% (e.g., at least 86%, 87%, 88%, 89%, 90%, 91%, 92%, 93%, 94%, 95%, 96%, 97%, 98%, or 99%) or 100% identity to the first Fc polypeptide sequence from Table 2C, wherein the second Fc polypeptide has at least 85% (e.g., at least 86%, 87%, 88%, 89%, 90%, 91%, 92%, 93%, 94%, 95%, 96%, 97%, 98%, or 99%) or 100% identity to the second Fc polylpeptide sequence from Table 2C, and wherein each of the first and second Fc polypeptides are further modified to contain a TfR binding site in the modified CH3 domain comprising: three, four, five, six, seven, or eight amino acid substitutions in a set of amino acid positions comprising 380 and 382-389 (e.g., F at position 382, Y or S at position 383, G, D, or E at position 384, D, G, N, or A at position 385, Q, S, or A at position 386, K at position 387, E or L at position 388, and N, T, or S at position 389); and five, six, or seven amino acid substitutions in a set of amino acid positions comprising 422, 424, 426, 433, 434, 438, and 440 (e.g., L at position 422, A at position 424, E at position 426, Y at position 438, and L at position 440), wherein the positions are determined according to EU numbering.

### Dimer That Binds TfR Monovalently

In some embodiments, the dimer is a monovalent Fc dimer that comprises two Fc polypeptides, in which only one of the two Fc polypeptides in the monovalent Fc dimer comprises a TfR-binding site, while the other Fc polypeptide does not bind to TfR. In addition, the Fc polypeptides can contain modifications for promoting heterodimzerization of the Fc dimer (e.g., T366W; and T366S, L368A, and Y407V). In some embodiments, a monovalent Fc dimer that specifically binds to TfR described herein comprises a first and second Fc polypeptide pair from Table 2D and the first Fc polypeptide is further modified to contain a TfR-binding site in the modified CH3 domain as described herein. In some embodiments, a monovalent Fc dimer that specifically binds to TfR described herein comprises a first and second Fc polypeptide pair from Table 2D and the second Fc polypeptide is further modified to contain a TfR-binding site in the modified CH3 domain as described herein.

In other embodiments, a monovalent Fc dimer that specifically binds to TfR described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the sequence from first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the first Fc polypeptides is further modified to contain a TfR-binding site in the modified CH3 domain as described herein.

In one embodiment, a monovalent Fc dimer that specifically binds to TfR described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the first Fc polypeptide is further modified to contain a TfR-binding site in the modified CH3 domain comprising: three, four, five, six, seven, or eight amino acid substitutions and/or one or two amino acid deletions in a set of amino acid positions comprising 380 and 382-389; and five, six, or seven amino acid substitutions in a set of amino acid positions comprising 422, 424, 426, 433, 434, 438, and 440, wherein the positions are determined according to EU numbering. In some embodiments, the substitutions and/or deletions are selected from: E, N, F, or Y at position 380, F at position 382, Y, S, A, or an amino acid deletion at position 383, G, D, E, or N at position 384, D, G, N, or A at position 385, Q, S, G, A, or N at position 386, K, I, R, or G at position 387, E, L, D, or Q at position 388, N, T, S, or R at position 389, L at position 422, A at position 424, E at position 426, H or E at position 433, N or G at position 434, Y at position 438, and L at position 440.

In one embodiment, a monovalent Fc dimer that specifically binds to TfR described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the first Fc polypeptide is further modified to contain a TfR-binding site in the modified CH3 domain comprising: three, four, five, six, seven, or eight amino acid substitutions in a set of amino acid positions comprising 380 and 382-389 (e.g., F at position 382, Y or S at position 383, G, D, or E at position 384, D, G, N, or A at position 385, Q, S, or A at position 386, K at position 387, E or L at position 388, and N, T, or S at position 389); and five, six, or seven amino acid substitutions in a set of amino acid positions comprising 422, 424, 426, 433, 434, 438, and 440 (e.g., L at position 422, A at position 424, E at position 426, Y at position 438, and L at position 440), wherein the positions are determined according to EU numbering.

In other embodiments, a monovalent Fe dimer that specifically binds to TfR described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the sequence from first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the second Fc polypeptides is further modified to contain a TfR-binding site in the modified CH3 domain as described herein.

In one embodiment, a monovalent Fc dimer that specifically binds to TfR described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the second Fc polypeptide is further modified to contain a TfR-binding site in the modified CH3 domain comprising: three, four, five, six, seven, or eight amino acid substitutions and/or one or two amino acid deletions in a set of amino acid positions comprising 380 and 382-389; and five, six, or seven amino acid substitutions in a set of amino acid positions comprising 422, 424, 426, 433, 434, 438, and 440, wherein the positions are determined according to EU numbering. In some embodiments, the substitutions and/or deletions are selected from: E, N, F, or Y at position 380, F at position 382, Y, S, A, or an amino acid deletion at position 383, G, D, E, or N at position 384, D, G, N, or A at position 385, Q, S, G, A, or N at position 386, K, I, R, or G at position 387, E, L, D, or Q at position 388, N, T, S, or R at position 389, L at position 422, A at position 424, E at position 426, H or E at position 433, N or G at position 434, Y at position 438, and L at position 440.

In one embodiment, a monovalent Fc dimer that specifically binds to TfR described herein comprises a first and second Fc polypeptide pair from Table 2D, wherein the first polypeptide has at least 85% (e.g., at least 86%, 87%, 88%, 89%, 90%, 91%, 92%, 93%, 94%, 95%, 96%, 97%, 98%, or 99%) or 100% identity to the first Fc polypeptide sequence from Table 2D, wherein the second polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the second Fc polypeptide is further modified to contain a TfR-binding site in the modified CH3 domain comprising: three, four, five, six, seven, or eight amino acid substitutions in a set of amino acid positions comprising 380 and 382-389 (e.g., F at position 382, Y or S at position 383, G, D, or E at position 384, D, G, N, or A at position 385, Q, S, or A at position 386, K at position 387, E or L at position 388, and N, T, or S at position 389); and five, six, or seven amino acid substitutions in a set of amino acid positions comprising 422, 424, 426, 433, 434, 438, and 440 (e.g., L at position 422, A at position 424, E at position 426, Y at position 438, and L at position 440), wherein the positions are determined according to EU numbering.

For example, the first Fc polypeptide from dimer pair K from Table 2D (i.e., SEQ ID NO:11) is further modified to comprise three, four, five, six, seven, or eight amino acid substitutions in a set of amino acid positions comprising 380 and 382-389 (e.g., F at position 382, Y or S at position 383, G, D, or E at position 384, D, G, N, or A at position 385, Q, S, or A at position 386, K at position 387, E or L at position 388, and N, T, or S at position 389); and five, six, or seven amino acid substitutions in a set of amino acid positions comprising 422, 424, 426, 433, 434, 438, and 440 (e.g., L at position 422, A at position 424, E at position 426, Y at position 438, and L at position 440), wherein the positions are determined according to EU numbering.

In one embodiment, a monovalent Fc dimer that specifically binds to TfR described herein comprises a first and second Fc polypeptide pair from Table 2D, wherein the first Fc polypeptide has at least 85% (e.g., at least 86%, 87%, 88%, 89%, 90%, 91%, 92%, 93%, 94%, 95%, 96%, 97%, 98%, or 99%) or 100% identity to the first Fc polypeptide sequence from Table 2D, wherein the second Fc polypeptide has at least 85% (e.g., at least 86%, 87%, 88%, 89%, 90%, 91%, 92%, 93%, 94%, 95%, 96%, 97%, 98%, or 99%) or 100% identity to the second Fc polypeptide sequence from Table 2D, and wherein the first Fc polypeptide is further modified to contain a TfR-binding site in the modified CH3 domain comprising a set of modifications selected from a row from Table 29 (e.g., TfR-binding site modifications from clones 42.2.19, 42.2.3-1H, 42.8.196, 42.8.80, 42.8.15, or 42.8.17 in Table 29). In one embodiment, a monovalent Fc dimer that specifically binds to TfR described herein comprises a first and second Fc polypeptide pair from Table 2D, wherein the first Fc polypeptide has at least 85% (e.g., at least 86%, 87%, 88%, 89%, 90%, 91%, 92%, 93%, 94%, 95%, 96%, 97%, 98%, or 99F) or 100 identity to the first Fc polypeptide sequence from Table 2D, wherein the second Fc polypeptide has at least 85 (e.g., at least 86, 87%, 88%, 89%, 90%, 91%, 92%, 93%, 94%, 95%, 96%, 97%, 98, or 99%) or 100% identity to the second Fc polypeptide sequence from Table 2D, and wherein the second Fc polypeptide is further modified to contain a TfR-binding site in the modified CH3 domain comprising a set of modifications selected from a row from Table 29 (e.g., TfR-binding site modifications from clones 42.2.19, 42.2.3-1H, 42.8.196, 42.8.80, 42.8.15, or 42.8.17 in Table 29).

In another aspect, the disclosure provides Fc polypeptide dimers having sequences of the first and second Fc polypeptides as listed in Tables 2C and 2D below:

TABLE 2C
Dimer Combinations For Bivalent CD98hc- or TfR Binding Site Modifications

| | First Fc | | |
|---|---|---|---|
| Dimer | Polypeptide: Second | First Fc | Second Fc |
| # | Fc Polypeptide | Polypeptide | Polypeptide |
| A | WT:WT | SEQ ID NO: 1 | SEQ ID NO: 1 |
| B | LALA:LALA | SEQ ID NO: 7 | SEQ ID NO: 7 |
| C | LALA-PG:LALA-PG | SEQ ID NO: 10 | SEQ ID NO: 10 |
| D | LALA-PS:LALA-PS | SEQ ID NO: 13 | SEQ ID NO: 13 |
| E | LS:LS | SEQ ID NO: 16 | SEQ ID NO: 16 |
| F | LALA-LS:LALA-LS | SEQ ID NO: 19 | SEQ ID NO: 19 |
| G | LALA-PG- | SEQ ID NO: 22 | SEQ ID NO: 22 |
| | LS:LALA:PG:LS | | |
| H | LALA-PS-LS:LALA:PS:LS | SEQ ID NO: 25 | SEQ ID NO: 25 |

TABLE 2D
Knob-Hole Dimer Combinations For Monovalent
CD98hc- or TfR Binding Site Modifications

| Dimer | First Fc Polypeptide:Second Fc | First Fc | Second Fc |
|---|---|---|---|
| # | Polypeptide | Polypeptide | Polypeptide |
| I | Knob:Hole | SEQ ID NO: 5 | SEQ ID NO: 6 |
| J | Knob-LALA:Hole:LALA | SEQ ID NO: 8 | SEQ ID NO: 9 |
| K | Knob-LALA-PG:Hole:LALA-PG | SEQ ID NO: 11 | SEQ ID NO: 12 |
| L | Knob-LALA-PS:Hole:LALA-PS | SEQ ID NO: 14 | SEQ ID NO: 15 |
| M | Knob-LS:Hole-LS | SEQ ID NO: 17 | SEQ ID NO: 18 |
| N | Knob-LALA-LS:Hole-LALA-LS | SEQ ID NO: 20 | SEQ ID NO: 21 |
| O | Knob-LALA-PG-LS:Hole:LALA- | SEQ ID NO: 23 | SEQ ID NO: 24 |
| | PG-LS | | |
| P | Knob-LALA-PS-LS:Hole:LALA- | SEQ ID NO: 26 | SEQ ID NO: 27 |
| | PS-LS | | |
| Q | Hole:Knob | SEQ ID NO: 6 | SEQ ID NO: 5 |
| R | Hole:LALA:Knob-LALA | SEQ ID NO: 9 | SEQ ID NO: 8 |
| S | Hole:LALA-PG:Knob-LALA-PG | SEQ ID NO: 12 | SEQ ID NO: 11 |
| T | Hole:LALA-PS:Knob-LALA-PS | SEQ ID NO: 15 | SEQ ID NO: 14 |
| U | Hole-LS:Knob-LS | SEQ ID NO: 18 | SEQ ID NO: 17 |
| V | Hole-LALA-LS:Knob-LALA-LS | SEQ ID NO: 21 | SEQ ID NO: 20 |
| W | Hole:LALA-PG-LS:Knob-LALA- | SEQ ID NO: 24 | SEQ ID NO: 23 |
| | PG-LS | | |
| X | Hole:LALA-PS-LS:Knob-LALA- | SEQ ID NO: 27 | SEQ ID NO: 26 |
| | PS-LS | | |

### XI. Conjugates

In some embodiments, a polypeptide (e.g., an Fc polypeptide) described herein is linked to an agent, e.g., an agent that is to be internalized into a cell and/or for transcytosis across an endothelium, such as the BBB, via a linker. The linker may be any linker suitable for joining an agent to the polypeptide. In some embodiments, the linkage is enzymatically cleavable. In certain embodiments, the linkage is cleavable by an enzyme present in the central nervous system.

In some embodiments, the linker is a peptide linker. The peptide linker may allow for the rotation of the agent and the polypeptide relative to each other; and/or is resistant to digestion by proteases. In some embodiments, the linker may be a flexible linker, e.g., containing amino acids such as Gly, Asn, Ser, Thr, Ala, and the like. Such linkers are designed using known parameters. For example, the linker may have repeats, such as Gly-Ser repeats.

In various embodiments, the conjugates can be generated using well-known chemical cross-linking reagents and protocols. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NHS) or its water soluble analog N-Hydroxysulfosuccinimide (Sulfo-NHS), Succinimidyl 4-(N-Maleimidomethyl)Cyclohexane-1-carboxylate (SMCC), m-maleimidobenzoyl-N-hydroxysuccinimide ester (MBS); N-succinimidyl (4-iodoacetyl) aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), and succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate·2HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers.

The agent of interest may be a therapeutic agent, including a cytotoxic agent, a DNA or RNA molecule, an antisense oligonucloetide, a chemical moiety, and the like. In some embodiments, the agent may be a peptide or small molecule therapeutic or imaging agent. In some embodiments, the small molecule is less than 1000 Da, less than 750 Da, or less than 500 Da.

The agent of interest may be linked to the N-terminal or C-terminal region of the CD98hc-binding polypeptide, or attached to any region of the polypeptide, so long as the agent does not interfere with binding of the CD98hc-binding polypeptide to CD98hc or CD98 heterodimer, i.e., CD98hc in complex with a CD98 light chain (LAT1 (SLC7A5), LAT2 (SLC7A8), y⁺LAT1 (SLC7A7), y⁺LAT2 (SLC7A6), Asc-1 (SLC7A10), or xCT (SLC7A11).

The agent of interest may be linked to the N-terminal or C-terminal region of the TfR-binding polypeptide, or attached to any region of the polypeptide, so long as the agent does not interfere with binding of the TfR-binding polypeptide to TfR.

### XII. Methods of Engineering Polypeptides to Bind CD98HC or TfR

In a further aspect, methods of engineering a modified CH3 domain to bind CD98hc are provided. In some embodiments, modification of a CH3 domain comprises substituting various amino acids relative to the sequence of SEQ ID NO:3 or to amino acids 111-217 of the sequence of SEQ ID NO:1. In some embodiments, the method comprises modifying a polynucleotide that encodes the modified CH3 domain polypeptide to incorporate amino acid changes, relative to the sequence of SEQ ID NO:3 or to amino acids 111-217 of the sequence of SEQ ID NO:1.

In some embodiments of engineering polypeptides to bind CD98hc, the method comprises modifying a polynucleotide that encodes the modified CH3 domain comprising a sequence having a first sequence comprising at least one substitution or deletion relative to the sequence of EWESNGQP (SEQ ID NO:52; 380 to position 387 of an Fc polypeptide (e.g., SEQ ID NO:1), EU numbering), (ii) a second sequence comprising at least one substitution relative to the sequence of NVFSCSVM (SEQ ID NO:53; 421 to position 428 of an Fc polypeptide (e.g., SEQ ID NO:1, EU numbering), and (iii) a third sequence comprising at least one substitution relative to the sequence of YTQKSLS (SEQ ID NO:53; 436 to position 442 of an Fc polypeptide (e.g., SEQ ID NO:1), EU numbering). In some embodiments, the method further comprises expressing and recovering a polypeptide comprising the modified CH3 domain; and determining whether the polypeptide binds to CD98hc.

In some embodiments of engineering polypeptides to bind TfR, the method comprises modifying a polynucleotide that encodes the modified CH3 domain comprising a sequence having a first sequence comprising at least one amino acid substitution and/or deletion relative to the sequence of AVEWESNGQPENN (SEQ ID NO:56), and (ii) a second sequence comprising at least one amino acid substitution in the sequence of VFSCSVMHEALHNHYTQKS (SEQ ID NO:57), in which the sequence of SEQ ID NO:56 is from position 378 to position 390 of an Fc polypeptide (e.g., SEQ ID NO:1), and the sequence of SEQ ID NO:57 is from position 422 to position 440 of an Fc polypeptide (e.g., SEQ ID NO:1), and the positions are determined according to EU numbering. In some embodiments, the method further comprises expressing and recovering a polypeptide comprising the modified CH3 domain; and determining whether the polypeptide binds to TfR.

The amino acids introduced into the desired positions may be generated by randomization or partial randomization to generate a library of CH3 domain polypeptides with amino acid substitutions at the various positions described herein. In some embodiments, the modified CH3 domain polypeptide is mutated in the context of an Fc region, which may or may not contain part of, or all of, a full hinge region.

The polypeptides comprising the modified CH3 domain may be expressed using any number of systems. For example, in some embodiments, polypeptides are expressed in a display system. In other illustrative embodiments, mutant polypeptides are expressed as soluble polypeptides that are secreted from the host cell. In some embodiments, the expression system is a display system, e.g., a viral display system, a cell surface display system such as a yeast display system, an mRNA display system, or a polysomal display system. The library is screened using known methodology to identify CD98hc binders, which may be further characterized to determine binding kinetics. Additional mutations may then be introduced into selected clones.

CD98hc-binding polypeptides of the present disclosure may have a broad range of binding affinities, e.g., based on the format of the polypeptide. For example, in some embodiments, a polypeptide comprising a modified CH3 domain has an affinity for CD98hc binding ranging anywhere from 1 μM to 10 μM. In some embodiments, the polypeptide binds human CD98hc with an affinity of 15 nM to 5 μM (e.g., 15 nM, 50 nM, 100 nM, 200 nM, 300 nM, 400 nM, 500 nM, 600 nM, 700 nM, 800 nM, 900 nM, 1 μM, 1.5 μM, 2 μM, 2.5 μM, 3 μM, 3.5 μM, 4 μM, 4.5 μM, or 5 μM). In another embodiment, the polypeptide binds to cynomolgus CD98hc with an affinity of 80 nM to 5 μM (e.g., 80 nM, 100 nM, 200 nM, 300 nM, 400 nM, 500 nM, 600 nM, 700 nM, 800 nM, 900 nM, 1 μM, 1.5 μM, 2 μM, 2.5 μM, 3 μM, 3.5 μM, 4 μM, 4.5 μM, or 5 μM). In some embodiments, affinity may be measured in a monovalent format. In other embodiments, affinity may be measured in a bivalent format.

TfR-binding polypeptides of the present disclosure may have a broad range of binding affinities, e.g., based on the format of the polypeptide. For example, in some embodiments, a polypeptide comprising a modified CH3 domain has an affinity for TfR binding ranging anywhere from 1 μM to 10 μM. In some embodiments, the polypeptide binds human TfR with an affinity of 15 nM to 10 μM (e.g., 15 nM, 50 nM, 100 nM, 200 nM, 300 nM, 400 nM, 500 nM, 600 nM, 700 nM, 800 nM, 900 nM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, or 10 μM). In another embodiment, the polypeptide binds to cynomolgus TfR with an affinity of 80 nM to 5 μM (e.g., 80 nM, 100 nM, 200 nM, 300 nM, 400 nM, 500 nM, 600 nM, 700 nM, 800 nM, 900 nM, 1 μM, 1.5 μM, 2 μM, 2.5 μM, 3 μM, 3.5 μM, 4 μM, 4.5 μM, or 5 μM). In some embodiments, affinity may be measured in a monovalent format. In other embodiments, affinity may be measured in a bivalent format.

Methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet® (FortéBio, Inc., Menlo Park, CA)), and Western blot analysis. In some embodiments, ELISA is used to determine binding affinity and/or cross-reactivity. Methods for performing ELISA assays are known in the art and are also described in the Example section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

### XIII. Nucleic Acids, Vectors, and Host Cells

The CD98hc-binding and TfR-binding polypeptides as described herein are typically prepared using recombinant methods. Accordingly, in some aspects, the disclosure provides isolated nucleic acids comprising a nucleic acid sequence encoding any of the polypeptides as described herein, and host cells into which the nucleic acids are introduced that are used to replicate the polypeptide-encoding nucleic acids and/or to express the polypeptides. In some embodiments, the host cell is eukaryotic, e.g., a human cell.

In another aspect, polynucleotides are provided that comprise a nucleotide sequence that encodes the polypeptides described herein. The polynucleotides may be single-stranded or double-stranded. In some embodiments, the polynucleotide is DNA (e.g., cDNA). In some embodiments, the polynucleotide is RNA.

In some embodiments, the polynucleotide is included within a nucleic acid construct. In some embodiments, the construct is a replicable vector. In some embodiments, the vector is selected from a plasmid, a viral vector, a phagemid, a yeast chromosomal vector, and a non-episomal mammalian vector.

In some embodiments, the polynucleotide is operably linked to one or more regulatory nucleotide sequences in an expression construct. In one series of embodiments, the nucleic acid expression constructs are adapted for use as a surface expression library (e.g., yeast or phage). In another series of embodiments, the nucleic acid expression constructs are adapted for expression of the polypeptide in a system that permits isolation of the polypeptide in milligram or gram quantities. In some embodiments, the system is a mammalian cell or yeast cell expression system.

Expression vehicles for production of a recombinant polypeptide include plasmids and other vectors. Any appropriate plasmid or vector can be used for this purpose, including those suitable for transient expression of polypeptides in eukaryotic cells. In some embodiments, it may be desirable to express the recombinant polypeptide by the use of a baculovirus expression system using appropriate vectors. Additional expression systems include adenoviral, adeno-associated virus, and other viral expression systems.

Vectors may be transformed into any suitable host cell. In some embodiments, the host cells, e.g., bacteria or yeast cells, may be adapted for use as a surface expression library. In some cells, the vectors are expressed in host cells to express relatively large quantities of the polypeptide. Such host cells include mammalian cells, yeast cells, insect cells, and prokaryotic cells. In some embodiments, the cells are mammalian cells, such as Chinese Hamster Ovary (CHO) cell, baby hamster kidney (BHK) cell, NS0 cell, Y0 cell, HEK293 cell, COS cell, Vero cell, or HeLa cell.

A host cell transfected with an expression vector encoding a CD98hc-binding or TfR-binding polypeptide can be cultured under appropriate conditions to allow expression of the polypeptide. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

### XIV. Methods of Delivery, Targeting, and Therapeutic Methods

A polypeptide described herein in accordance with the disclosure may be used therapeutically in many indications. In some embodiments, the polypeptide is used to deliver a therapeutic agent to a target cell type that expresses CD98hc or TfR. In some embodiments, the polypeptide may be used to transport a therapeutic moiety across an endothelium, e.g., the BBB, to be taken up by the brain. Thus, a polypeptide of the present disclosure may be used, e.g., conjugated to a therapeutic agent, to deliver the therapeutic agent to treat a neurological disorder such as a disease of the brain or central nervous system (CNS), to treat a cancer, to treat an autoimmune or inflammatory disease, or a cardiovascular disease.

In some embodiments, provided herein are methods of targeting extracellular targets in the brain using a polypeptide of the present disclosure. In some embodiments, the polypeptide of the present disclosure is transported across the BBB and into the parenchyma without being transcytosed into a cell within the brain. In some embodiments, the method comprises the delivery of a therapeutic agent across the BBB to an extracellular target on or near an astrocyte, microglia, oligodendrocyte, or a cancer cell. In other embodiments, the extracellular target is an antigen in the brain, such as a plaque, tangle, or other non-cellular target. In some embodiments, targeted delivery is to an extracellular target on a microglia. In some embodiments, targeted delivery is to an extracellular target on a cancer cell.

In some embodiments, provided herein are methods of treating a disease in the brain of a patient, the method comprising the delivery of a therapeutic agent to an extracellular targets in the brain using a polypeptide of the present disclosure. In some embodiments, the method comprises the delivery of the therapeutic agent across the BBB and into the parenchyma without being transcytosed into a cell within the brain. In some embodiments, the method comprises the delivery of a therapeutic agent across the BBB to an extracellular target on or near an astrocyte, microglia, oligodendrocyte, or a cancer cell. In other embodiments, the extracellular target is an antigen in the brain, such as a plaque, tangle, or other non-cellular target (e.g., Abeta, Tau or alpha-synuclein). In some embodiments, the disease of the brain to be treated is selected from the group consisting of Frontotemporal Dementia, Amyotrophic Lateral Sclerosis, Alzheimer's Disease, and Parkinson's Disease. In some embodiments, the cancer is glioblastoma or metastatic cancer in the brain.

A polypeptide of the present disclosure is administered to a subject at a therapeutically effective amount or dose. The dosages may be varied according to several factors, including the chosen route of administration, the formulation of the composition, patient response, the severity of the condition, the subject's weight, and the judgment of the prescribing physician. The dosage can be increased or decreased over time, as required by an individual patient. In some embodiments, a patient initially is given a low dose, which is then increased to an efficacious dosage tolerable to the patient. Determination of an effective amount is well within the capability of those skilled in the art.

In various embodiments, a polypeptide of the present disclosure is administered parenterally (e.g., intraperiotneally, subcutaneously, intradermally, or intramuscularly). In some embodiments, the polypeptide is administered intravenously. Intravenous administration can be by infusion or as an intravenous bolus. Combinations of infusion and bolus administration may also be used. In other embodiments, a polypeptide may be administered orally, by pulmonary administration, intranasal administration, intraocular administration, or by topical administration. Pulmonary administration can also be employed, e.g., by use of an inhaler or nebulizer, and formulation with an aerosolizing agent.

### XV. Pharmaceutical Compositions and Kits

In another aspect, pharmaceutical compositions and kits comprising a polypeptide in accordance with the disclosure are provided.

### Pharmaceutical Compositions

Guidance for preparing formulations for use in the present disclosure can be found in any number of handbooks for pharmaceutical preparation and formulation that are known to those of skill in the art.

In some embodiments, a pharmaceutical composition comprises a polypeptide described herein and further comprises one or more pharmaceutically acceptable carriers and/or excipients. A pharmaceutically acceptable carrier includes any solvents, dispersion media, or coatings that are physiologically compatible and that preferably does not interfere with or otherwise inhibit the activity of the active agent. Various pharmaceutically acceptable excipients are well-known.

In some embodiments, the carrier is suitable for intravenous, intrathecal, intramuscular, oral, intraperitoneal, transdermal, topical, or subcutaneous administration. Pharmaceutically acceptable carriers can contain one or more physiologically acceptable compounds that act, for example, to stabilize the composition or to increase or decrease the absorption of the polypeptide. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are also available in the art.

The pharmaceutical compositions described herein can be manufactured in a manner that is known to those of skill in the art, e.g., by means of conventional mixing, dissolving, granulating, dragee-making, emulsifying, encapsulating, entrapping, or lyophilizing processes.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. The determination of the appropriate dosage or route of administration can be determined by one of skill in the art.

### Kits

In some embodiments, kits comprising a polypeptide described herein are provided. In some embodiments, the kits are for use in preventing or treating a neurological disorder such as a disease of the brain or central nervous system (CNS).

In some embodiments, the kit further comprises one or more additional therapeutic agents. For example, in some embodiments, the kit comprises a polypeptide as described herein and further comprises one or more additional therapeutic agents for use in the treatment of a neurological disorder. In some embodiments, the kit further comprises instructional materials containing directions (i.e., protocols) for the practice of the methods described herein (e.g., instructions for using the kit for administering a composition across the BBB). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### XVI. EXAMPLES

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results. Efforts have been made to ensure accuracy with respect to numbers used (e.g., amounts, temperatures, etc.), but some experimental error and deviation may be present. The practice of the present disclosure will employ, unless otherwise indicated, conventional methods of protein chemistry, biochemistry, recombinant DNA techniques and pharmacology, within the skill of the art. Such techniques are explained fully in the literature. Additionally, it should be apparent to one of skill in the art that the methods for engineering as applied to certain libraries can also be applied to other libraries described herein.

### Example 1. Generation of Limited Liability Libraries and Selecting CD98HC-Binding Clones for Engineering

We developed a beta-sheet library that was used for discovery of polypeptides capable of binding CD98hc. The library includes randomization at human Fc residues 380, 382, 384-387, 422, 424, 426, 438 and 440 (EU numbering). We have observed that large libraries (e.g., more than 9 residue positions) can produce clones that are non-specific and/or not well behaved because they have large numbers of unfavorable residues, often in adjacent positions. To reduce the frequency of residues that can create such liabilities (in particular, cysteine, arginine, tryptophan, and glycine as noted below), the library was engineered using a codon bias to limit the frequency and position of these residues. The libraries that implement this technology are called “limited liability” libraries. These disfavored residues include arginine and tryptophan, which can enhance the affinity of an interaction, but can also enhance non-specificity (entropic interactions); cysteine, which is an oxidation liability; and glycine, which increases the flexibility of protein structure and can destabilize secondary structure. To avoid these four residues at defined positions, we introduced the NHK codon into our libraries. By alternating NNK (allows all 20 amino acids) and NHK codons, this limits these four amino acids from being at neighboring positions without overly limiting the diversity of the library. This approach resulted in increased quality of the polypeptides identified in the library.

Taking the approach outlined above, a library was designed to limit the inclusion of liability residues at adjacent sites or in total, as follows in Table 2E. The libraries were generated using Kunkel mutagenesis with a mixture 6 oligos using the WT Fc gene in a phage display vector. The phage library called “LLB”, for Limited Liability B, was screened for binding to human CD98hc. The screening of these libraries resulted in five clones that bound weakly to human CD98hc (shown in Table A1), called LLB1, LLB2, and LLB3, LLB6, LLB7, which were selected for further engineering. LLB2 and LLB3 were found to be part of the same family (called LLB2). Clones LLB1, LLB6 and LLB7 were found to be part of the same family (called LLB1).

TABLE 2E
Oligos were mixed together resulting in 9
different library variants that were pooled

| Fc (EU numbering) | 380 | 382 | 384 | 385 | 386 | 387 |
|---|---|---|---|---|---|---|
| Front_oligo_1 | NHK | NNK | NHK | NNK | NHK | NNK |
| Front_oligo_2 | NNK | NHK | NNK | NHK | NNK | NHK |
| Front_oligo_3 | NHK | NHK | NHK | NHK | NHK | NHK |

| Fc EU numbering | 422 | 424 | 426 | 438 | 440 |
|---|---|---|---|---|---|
| Back oligo_1 | NNK | NHK | NNK | NHK | NNK |
| Back oligo_2 | NHK | NNK | NHK | NNK | NHK |
| Back oligo_3 | NHK | NHK | NHK | NHK | NHK |

TABLE A1
Clones of the LLB1 and LLB2 families

| | 369 | 370 | 371 | 372 | 373 | 374 | 375 | 376 | 377 | 378 | 379 | 380 | 381 | 382 |
|---|---|---|---|---|---|---|---|---|---|---|---|---|---|---|
| WT Fc | V | K | G | F | Y | P | S | D | I | A | V | E | W | E |
| LLB1 | | | | | | | | | | | | D | | R |
| LLB2 | | | | | | | | | | | | L | | N |
| LLB3 | | | | | | | | | | | | L | | N |
| LLB6 | | | | | | | | | | | | D | | R |
| LLB7 | | | | | | | | | | | | D | | N |
| | 383 | 384 | 385 | 386 | 387 | 388 | 389 | 390 | 391 | 392 | 418 | 419 | 420 | 421 |
| WT Fc | S | N | G | Q | P | E | N | N | Y | K | Q | Q | G | N |
| LLB1 | | Y | Y | T | R | | | | | | | | | |
| LLB2 | | K | F | E | L | | | | | | | | | |
| LLB3 | | R | F | F | I | | | | | | | | | |
| LLB6 | | Y | F | P | F | | | | | | | | | |
| LLB7 | | Y | L | F | T | | | | | | | | | |

| | 422 | 423 | 424 | 425 | 426 | 427 | 428 | 429 | 430 | 431 | 432 | 433 | 434 |
|---|---|---|---|---|---|---|---|---|---|---|---|---|---|
| WT Fc | V | F | S | C | S | V | M | H | E | A | L | H | N |
| LLB1 | K | | V | | D | | | | | | | | |
| LLB2 | L | | A | | N | | | | | | | | |
| LLB3 | I | | A | | N | | | | | | | | |
| LLB6 | L | | L | | D | | | | | | | | |
| LLB7 | I | | W | | | | | | | | | | |

| | 435 | 436 | 437 | 438 | 439 | 440 | 441 | 442 | 443 | 444 | 445 | 446 | 447 |
|---|---|---|---|---|---|---|---|---|---|---|---|---|---|
| WT Fc | H | Y | T | Q | K | S | L | S | L | S | P | G | K |
| LLB1 | | | | I | | T | | | | | | | |
| LLB2 | | | | F | | N | | | | | | | |
| LLB3 | | | | F | | N | | | | | | | |
| LLB6 | | | | V | | P | | | | | | | |
| LLB7 | | | | F | | K | | | | | | | |

### Example 2. Generation of LLB2 Family CD98HC Binders

### LLB2 Family Affinity Maturation (AM1-AM4)

Two affinity maturation libraries (LLB2-AM1 and LLB2-AM2, Table 3) were designed for LLB2 and LLB3 clones as follows using Kunkel mutagenesis. Codons were chosen to include residues from the LLB2 and LLB3 hits to increase the library positions screened based on residues that are helpful for affinity maturation of these libraries. The libraries were screened using phage display to human CD98hc which resulted in 13 unique clones from these libraries that had affinity to human and cyno CD98hc (measured by surface plasmon resonance (SPR) using a Biacore™ machine). The affinity of the clones is shown in Table 9A and the sequences of the clones are shown in Table A2. The affinity of these clones was measured with anti-BACE1 Fabs having full effector function (effector+) (i.e., no modifications for modulating effector function, such as LALA or PG/PS) and in a bivalent format (i.e., no knob or hole mutations). Cell binding of the clones was also tested in HeLa cells for huCD98hc binding, CHO:cyCD98hc cells for cyno binding, and CHO cells as a negative control.

TABLE 3
Libraries LLB2-AM1 and LLB2-AM2

| Name | Type | 380 | 382 | 384 | 385 | 386 | 387 |
|---|---|---|---|---|---|---|---|
| LLB2-AM1 | AA | X | X | X | X | X | X |
| | codons | NNK | NNK | NNK | NNK | NNK | NNK |
| LLB2-AM2 | AA | L | N | KRNS | F | EFYDV | LIVF |
| | codons | CTG | AAT | ARN | TTT | KWN | NTY |

| Name | Type | 422 | 424 | 426 | 428 | 434 | 438 | 440 |
|---|---|---|---|---|---|---|---|---|
| LLB2-AM1 | AA | LIV | A | N | ML | NS | F | N |
| | codons | VTH | GCC | AAT | MTG | ARY | TTT | AAT |
| LLB2-AM2 | AA | X | X | X | ML | NS | X | X |
| | codons | NNK | NNK | NNK | MTG | ARY | NNK | NNK |

X = any amino acid

The affinity to human and cyno CD98hc was further engineered and improved for the LLB2 family using two additional libraries that were created using LLB2-10 as a background with additional mutations and positions to further explore the sequence space (Table 4). 21 clones were selected using phage display to human CD98hc from these libraries. The sequences of the selected clones are shown in Table A3 and their affinity to human and cyno CD98hc (measured by surface plasmon resonance (SPR) using a Biacore™ machine) is shown in Table 9A. The affinity of the clones was measured with anti-BACE1 Fabs having full effector function and in a bivalent format (i.e., no knob and hole mutations). Cell binding of the clones was also tested in HeLa cells for huCD98hc binding, CHO:cyCD98hc cells for cyno binding, and CHO cells as a negative control.

TABLE 4
Libraries LLB2-AM3, and LLB2-AM4

| Name | Type | 378 | 380 | 382 | 383 | 384 | 385 | 386 | 387 | 389 | 391 |
|---|---|---|---|---|---|---|---|---|---|---|---|
| LLB2- | AA | X | L | N | X | R | F | VASL | L | X | X |
| AM3 | | | | | | | | | | | |
| | Codons | NHK | CTG | AAT | NHK | CGC | TTT | KYR | CTG | NHK | NHK |
| LLB2- | AA | ● | L | X | X | X | F | X | L | ● | ● |
| AM4 | | | | | | | | | | | |
| | Codons | ● | CTG | NHK | NHK | NHK | TTT | NHK | CTG | ● | ● |

| Name | Type | 421 | 422 | 424 | 426 | 428 | 436 | 438 | 440 | 441 | 442 |
|---|---|---|---|---|---|---|---|---|---|---|---|
| LLB2- | AA | X | I | A | N | L | X | F | N | ● | X |
| AM3 | | | | | | | | | | | |
| | Codons | NHK | ATC | GCC | AAT | CTG | NHK | TTT | AAT | ● | NHK |
| LLB2- | AA | ● | I | A | N | L | ● | F | N | LP | X |
| AM4 | | | | | | | | | | | |
| | Codons | ● | ATC | GCC | AAT | NHK | ● | TTT | NHK | CYN | NHK |

● = WT residue and codon
X = All amino acids except Arg, Trp, Gly, Cys

At the same time as selection with LLB2-AM3 and LLB2-AM4, the clone LLB2-10 background was used to make rational design changes which included residues predicted to further improve the affinity to human and cyno CD98hc. The clone sequences are shown in Table A4 and the affinity of the clones (measured by surface plasmon resonance (SPR) using a Biacore™ machine) is shown in Table 9A. The affinity of the clones was measured with anti-BACE1 Fabs having full effector function and in a bivalent format (i.e., no knob or hole mutations). Clones LLB2-10-5, 2-10⁻⁶ and 2-10⁻⁸ were converted to a monovalent format and tested for affinity to CD98hc by SPR using a Biacore™ machine with anti-BACE1 Fabs having full effector function on both sides and with a CD98hc binding site on the knob side (and no CD98hc binding site on the hole side). The valency of the CD98hc-binding molecule did not have a great impact on the affinity to CD98hc, i.e., less than a 2-fold difference. Cell binding of the clones was also tested in HeLa cells for huCD98hc binding, CHO:cyCD98hc cells for cyno binding, and CHO cells as a negative control.

### Yeast Display LLB2 Soft Library

The ability to use yeast display to select for well-behaved clones using higher surface expression levels was leveraged to improve the properties of the LLB2 family. In order to achieve improved properties for this family, a soft mutagenesis library was made (70:10:10:10 oligo bias) (Table 5). To this end, the desired codon with a mix of 70% of the original base, with 10% of each of the other three bases was used. This resulted in approximately 50% of the original amino acids with the remainder being a mixture of the other amino acids. The original LLB2 clone backbone was used for soft mutagenesis, except residue L380 was soft mutagenized to the wild-type Glu residue and M428 was soft mutagenized to Leu residue. The library was assembled by two step PCR and yeast homologous recombination. This library was then displayed on the surface of yeast and the highest 20% of expressing clones that also bound tightly to human CD98hc were selected. This screening resulted in 9 clones that were tested for binding by surface plasmon resonance (SPR) using a Biacore™ machine to CD98hc, see Table A5. The affinity of the clones was measured by surface plasmon resonance (SPR) using a Biacore™ machine with non-binding Fabs having full effector function and in a bivalent format (i.e., no knob and hole mutations). Cell binding of the clones was also tested in HeLa cells for huCD98hc binding, CHO:cyCD98hc cells for cyno binding, and CHO cells as a negative control.

TABLE 5
LLB2 soft mutagenesis library

| | 380 | 382 | 384 | 385 | 386 | 387 | 422 | 424 | 426 | 428 | 438 | 440 |
|---|---|---|---|---|---|---|---|---|---|---|---|---|
| WT Fc | E | E | N | G | Q | P | V | S | S | M | Q | S |
| residues | | | | | | | | | | | | |
| LLB2 Soft | E | N | K | F | E | L | L | A | N | L | F | N |
| Library* | | | | | | | | | | | | |

*Underlined residues were made with 70:10:10:10 nucleotide mixtures, where 70% is the original base, and 10% were the other three bases.

### Rational Design to Improve the PK of the LLB2 Family

The M428L mutation in various LLB2 backbones appeared to contribute to a poor HIC profile and faster clearance in wild-type mice. This may be because of instability, poor specificity, and/or a difference in the interaction with mouse FcRn compared to a wild-type IgG. Therefore, the clones in Table A6 were designed using previous clones further engineered with a M428Y mutation in the LLB2 background mutation. Additionally, these clones also have a E380L mutation. These clones were made with non-binding Fabs having full effector function and in a monovalent format (i.e., CD98hc binding site on the knob side, with no CD98hc binding site on the hole side).

### LLB2-10-8 Patch Libraries for Affinity Maturation of Binding to CD98hc

The LLB2-10-8 lead clone was affinity matured using mutagenesis of 4-5 positions to every amino acid (NNK) in patches around the structure. This was done to optimize each region separately and to build a consensus for the best sequence. The 10 libraries are shown below in Table 6 with NNK signifying that the residue was mutated in the background of the LL2-10⁻⁸ clone. The library was assembled by two step PCR and yeast homologous recombination. The top 24 clones were picked for affinity measurement by surface plasmon resonance (SPR) using a Biacore™ machine. The affinity of the clones is shown in Table 9A and the sequences of the clones are shown in Table A7. The clones were made with non-binding Fabs having full effector function and in a monovalent format (i.e., CD98hc binding site on the knob side and no CD98hc binding site on the hole side). Cell binding of the clones was also tested in HeLa cells for huCD98hc binding, CHO:cyCD98hc cells for cyno binding, and CHO cells as a negative control.

TABLE 6
LLB2 Affinity Dematuration/Rational Design Library

| | 380 | 382 | 383 | 384 | 385 | 386 | 387 | 389 | 390 |
|---|---|---|---|---|---|---|---|---|---|
| WT Fc | E | E | S | N | G | Q | P | N | N |
| LLB2- | L | N | S | R | F | V | L | N | N |
| 10-8 | | | | | | | | | |
| Patch. 1 | | NNK | | | | | | | |
| Patch. 2 | | | NNK | NNK | NNK | NNK | | | |
| Patch. 3 | | | NNK | | | NNK | NNK | NNK | NNK |
| Patch. 4 | | | | | | | | | |
| Patch. 5 | | NNK | | NNK | | | | | |
| Patch. 6 | | | NNK | | | | | NNK | |
| Patch. 7 | | NNK | NNK | NNK | | | | | |
| Patch. 8 | | | | NNK | | NNK | | | |
| Patch. 9 | | | | | NNK | | | | |
| Patch. 10 | | | NNK | | | | | | |
| | 421 | 422 | 424 | 426 | 428 | 436 | 438 | 440 | 442 |
| WT Fc | N | V | S | S | M | Y | Q | S | S |
| LLB2- | E | I | A | N | Y | Y | F | N | A |
| 10-8 | | | | | | | | | |
| Patch. 1 | NNK | NNK | | | | | | NNK | |
| Patch. 2 | | | | | | | | | |
| Patch. 3 | | | | | | | | | |
| Patch. 4 | | | | | | NNK | NNK | NNK | NNK |
| Patch. 5 | | | NNK | NNK | | | | | |
| Patch. 6 | NNK | | | | | NNK | | | NNK |
| Patch. 7 | | | NNK | | | | | | |
| Patch. 8 | | NNK | | | | NNK | | | NNK |
| Patch. 9 | | NNK | | | | | NNK | NNK | |
| Patch. 10 | | | | NNK | | | | NNK | NNK |

### LLB2-10-8 Patch Libraries

In order to obtain affinity variants that bound with a weaker affinity to CD98hc than the LLB2-10-8 clone, single, double, or triple amino acid mutations were made to residues that were previously observed to bind to CD98hc at a weaker affinity (Table 7; Table A8). The variants were cloned, expressed, purified and tested for their affinity to human and cyno CD98hc by surface plasmon resonance (SPR) using a Biacore™ machine. The affinity of the clones is shown in Table 9A. Combining these mutations allowed for development of variants with a greater affinity range. Together, the LLB2 family has clones that span K_{d} values from 15 nM to 5 μM for human CD98hc and 80 nM to 5 μM for cyno CD98hc. Cell binding of the clones was also tested in HeLa cells for huCD98hc binding, CHO:cyCD98hc cells for cyno binding, and CHO cells as a negative control.

TABLE 7
Diversity used for rational design of LLB2

| | 380 | 382 | 384 | 385 | 386 | 387 | 421 | 422 |
|---|---|---|---|---|---|---|---|---|
| WT Fc | E | E | N | G | Q | P | N | V |
| LLB2- | L | N | X | X | X | L | X | X |
| 10-8 | | | | | | | | |
| AA used | | | HR | FY | EV | | NE | IVT |

| | 424 | 426 | 428 | 436 | 438 | 440 | 442 |
|---|---|---|---|---|---|---|---|
| WT Fc | S | S | M | Y | Q | S | S |
| LLB2- | A | N | Y | X | F | N | X |
| 10-8 | | | | | | | |
| AA used | | | | YWR | | | SARKH |

In order to obtain further variants in the 600-5000 nM affinity range, a second dematuration was performed using LLB2-10⁻⁸-d6, LLB2-10⁻⁸-d12, and LLB2-10⁻⁸-d18 clones as a templates except for the following changes that were predicted to decrease the affinity to human CD98hc based on previous data. Position 382 was kept as N or changed to S, position 385 alternated between Y or F, position 386 was alternated between V, E, Q, and position 387 was kept as L or alternate to P. Variants were made in combinations that had not been tested previously. Variants that bound with measurable affinity from this round of engineering are shown in Table A12 and the corresponding human binding affinity by surface plasmon resonance (SPR) using a Biacore™ machine are shown in Table 9B. Cell binding of the clones was also tested in HeLa cells for huCD98hc binding and CHO cells as a negative control.

### Consensus of LLB2 Family Amino Acids

Below in Table 8 are the residues that allow for binding to CD98hc in the LLB2 family.

TABLE 8
Amino acids that are acceptable in the LLB2 family

| | 380 | 382 | 384 | 385 | 386 | 387 | 421 | 422 | 424 | 426 | 428 | 436 | 438 | 440 | 442 |
|---|---|---|---|---|---|---|---|---|---|---|---|---|---|---|---|
| WT | E | E | N | G | Q | P | N | V | S | S | M | Y | Q | S | S |
| FC | | | | | | | | | | | | | | | |
| | L | N | R | F | V | L | E | I | A | N | Y | Y | F | N | A |
| | | | H | Y | L | | N | V | | S | W | R | W | | Q |
| | | | Q | | I | | Q | T | | | | W | | | K |
| | | | | | F | | A | P | | | | | | | R |
| | | | | | Y | | | | | | | | | | H |
| | | | | | E | | | | | | | | | | M |
| | | | | | | | | | | | | | | | S |

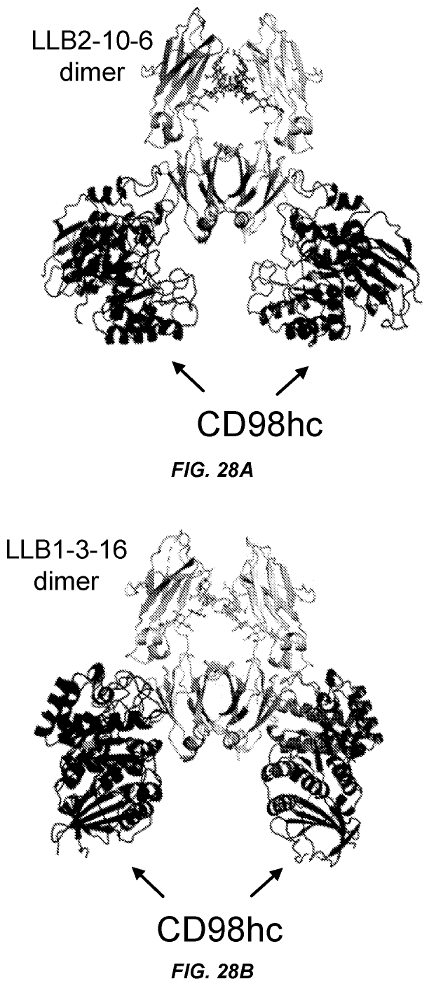

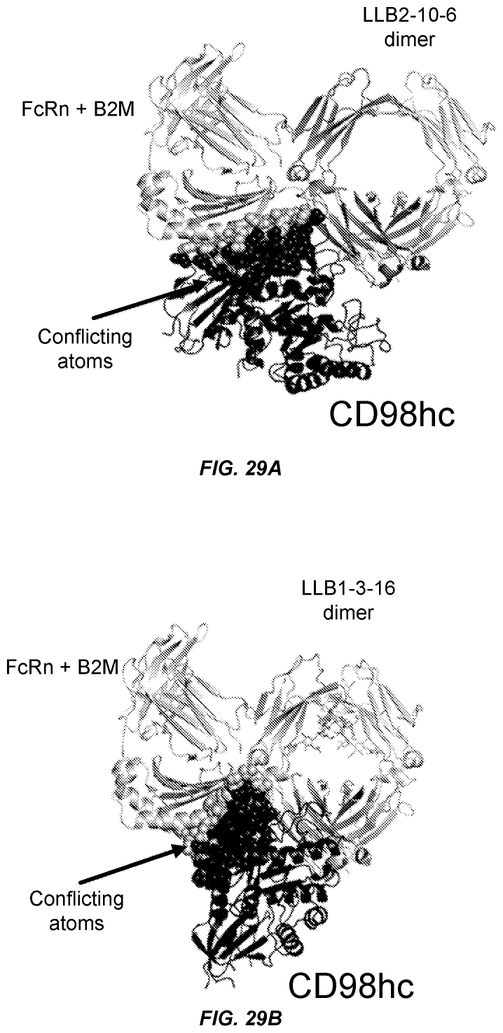

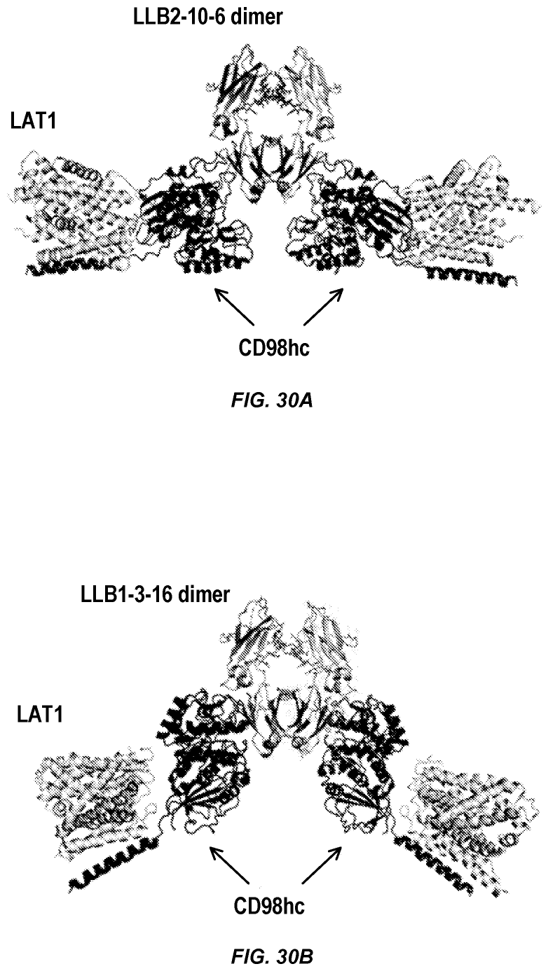

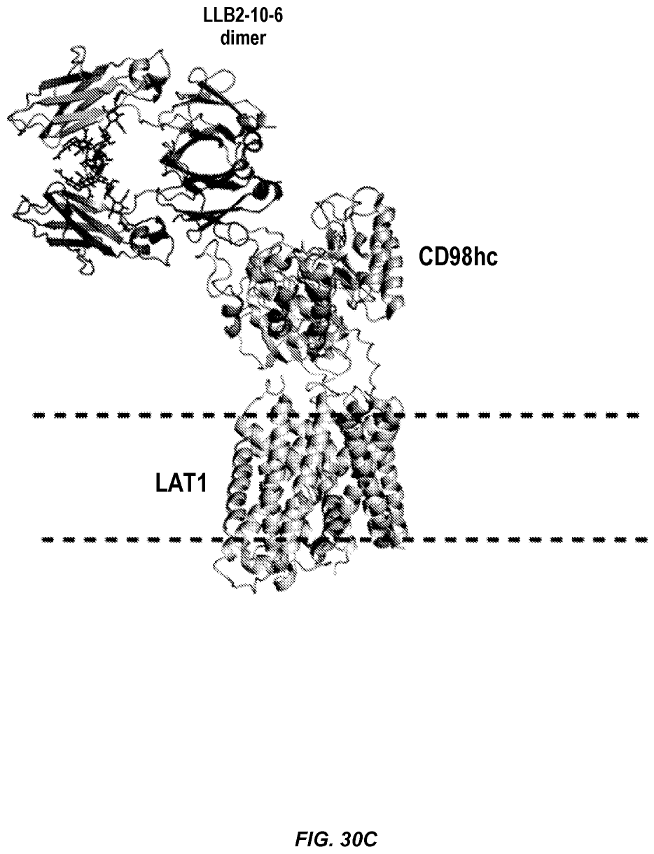

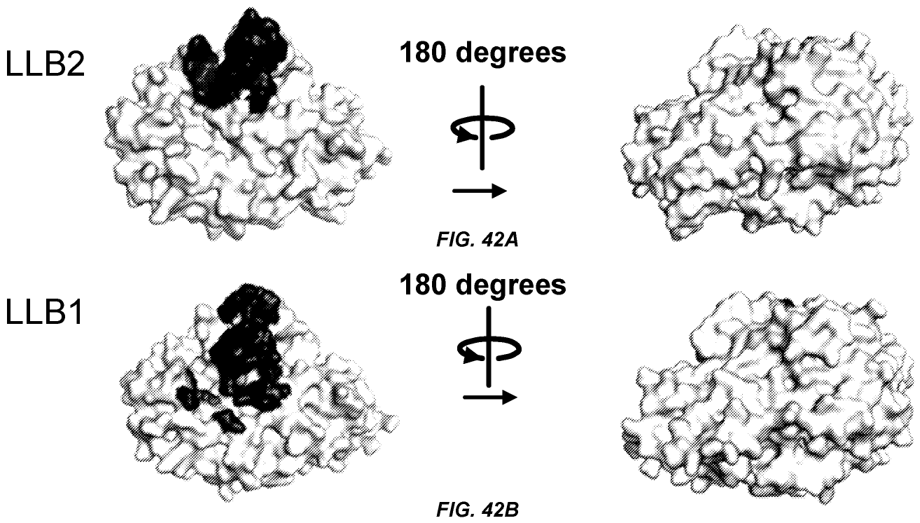

To understand the nature of the interaction between the CD98hc binders described herein and CD98hc, a bivalent LLB2-10⁻⁶ CD98hc binder and bivalent LLB1-3-16 CD98hc binder (both without any Fabs attached) were each co-crystalized with human CD98hc extracellular domain (ECD) at 2.25 A resolution. The structure shows that the CD98hc binders binds to CD98hc at the engineered surface to an epitope on CD98hc of the structured loop region next to the alpha/beta barrel structure, (FIGS. 28A and 28B). The CD98hc epitope for LLB2 is shown in FIG. 42A and the CD98hc epitope for LLB1 is shown in FIG. 42B. Additionally, a model of how FcRn binds to the CD98hc binders in the presence and absence of CD98hc was generated using the crystal structure and suggests that FcRn can bind in the absence of CD98hc but not in the presence of CD98hc (FIGS. 29A and 29B). A model of the interaction of the CD98hc binders with CD98hc in complex with LAT1 at the membrane was also generated. The model of the monovalent CD98hc binders bound to the CD98hc/LAT1 complex shows that the CD98hc binder can bind easily to CD98hc on the surface (FIG. 30C). Furthermore, the model suggests that one bivalent TV can bind to two CD98hc/LAT1 complex, albeit at an extreme angle of opposing membranes (FIGS. 30A and 301B).

TABLE 9A
Affinity measurement for LLB2 variants

| | Human CD98hc | Cyno CD98hc |
|---|---|---|
| | (nM) | (nM) |
| LLB2 | Not tested | Not tested |
| LLB3 | Not tested | Not tested |
| LLB2-1 | 950 | 4300 |
| LLB2-2 | 1300 | Not tested |
| LLB2-3 | 1200 | Not tested |
| LLB2-4 | 1700 | Not tested |
| LLB2-5 | 1900 | Not tested |
| LLB2-6 | Not Determinable | Not tested |
| LLB2-7 | 3600 | Not tested |
| LLB2-8 | 790 | 3200 |
| LLB2-9 | 750 | 3300 |
| LLB2-10 | 680 | 2700 |
| LLB2-11 | 1300 | 8200 |
| LLB2-12 | 990 | 5000 |
| LLB2-13 | 850 | 3800 |
| LLB2-17 | 2000 | 9200 |
| LLB2-18 | 1800 | 9100 |
| LLB2-19 | 9200 | >10000 |
| LLB2-20 | 1200 | 5500 |
| LLB2-21 | 420 | 2300 |
| LLB2-22 | Not tested | Not tested |
| LLB2-23 | 500 | 2600 |
| LLB2-24 | 1100 | 6200 |
| LLB2-25 | Not tested | Not tested |
| LLB2-26 | Not tested | Not tested |
| LLB2-27 | No binding | No binding |
| LLB2-28 | Not tested | Not tested |
| LLB2-29 | No binding | No binding |
| LLB2-30 | 450 | 2000 |
| LLB2-31 | 640 | 2100 |
| LLB2-32 | No binding | No binding |
| LLB2-33 | Not tested | Not tested |
| LLB2-34 | No binding | No binding |
| LLB2-35 | Not tested | Not tested |
| LLB2-36 | Not tested | Not tested |
| LLB2-37 | 610 | 2800 |
| LLB2-10-1 | 795 | 4200 |
| LLB2-10-2 | Not tested | Not tested |
| LLB2-10-3 | Not tested | Not tested |
| LLB2-10-4 | 630 | 3000 |
| LLB2-10-5 | 390 | 2200 |
| LLB2-10-6 | 460 | 2100 |
| LLB2-10-7 | 1100 | 6200 |
| LLB2-10-8 | 170 | 890 |
| LLB2-10-9 | 250 | 1300 |
| LLB2-10-10 | 220 | 1100 |
| LLB2-10-11 | 230 | 1200 |
| LLB2-10-12 | 210 | 1200 |
| LLB2-10-13 | 260 | 1100 |
| LLB2-10-5 Mono | 280 | 1700 |
| LLB2-10-6 Mono | 420 | 2100 |
| LLB2-10-8 Mono | 190 | 1100 |
| LLS2.33.2 | 2000 | 4500 |
| LLS2.33.3 | 5400 | >20000 |
| LLS2.33.8 | 2200 | 8200 |
| LLS2.33.5 | 1700 | 6900 |
| LLS2.43.5 | 1200 | 5900 |
| LLB2.C.1 | issue | 1500 |
| LLB2.C.3 | 1000 | 3500 |
| LLS2.C.4 | 700 | 2700 |
| LLS2.C.15 | 620 | 2200 |
| LLB2-10-8-1 | >20000 | No binding |
| LLB2-10-8-3 | 200 | 1200 |
| LLB2-30 | 250 | 1200 |
| LLB2-31 | 270 | 1200 |
| LLB2-37 | 390 | 2000 |
| LLB2-10-9 | 180 | 870 |
| LLB2-10-11 | 130 | 780 |
| LLB2-10-12 | 140 | 790 |
| LLB2-10-13 | 200 | 1000 |
| LLB2.10.8.2.1 | 110 | 490 |
| LLB2.10.8.12.1 | 115 | 526 |
| LLB2.10.8.2.3 | 150 | 760 |
| LLB2.10.8.2.4 | 133 | 970 |
| LLB2.10.8.12.5 | 230 | 1030 |
| LLB2.10.8.2.15 | 175 | 930 |
| LLB2.10.8.4.15 | 120 | 870 |
| LLB2.10.8.4.8 | 24 | 260 |
| LLB2.10.8.4.12 | 15 | 80 |
| LLB2.10.8.14.3 | 20 | 200 |
| LLB2.10.8.14.4 | 44 | 440 |
| LLB2.10.8.14.12 | 39 | 380 |
| LLB2.10.8.5.1 | 160 | 790 |
| LLB2.10.8.5.3star | Weak | Weak |
| LLB2.10.8.18.5 | 38 | 400 |
| LLB2.10.8.18.14 | 44 | 300 |
| LLB2.10.8.9.11 | 150 | 830 |
| LLB2.10.8.10.1 | 54 | 300 |
| LLB2.10.8.10.3 | 43 | 240 |
| LLB2.10.8.10.8 | 94 | 490 |
| LLB2.10.8.21 | 35 | 350 |
| LLB2.10.8.22 | 48 | 410 |
| LLB2.10.8.23 | 28 | 270 |
| LLB2.10.8.24 | 16 | 110 |
| LLB2.10.8.12.5.N | 1500 | 4700 |
| LLB2.10.8.4.12.N | 140 | 700 |
| LLB2.10.8.14.3.N | 270 | 1900 |
| LLB2.10.8.14.4.N | 350 | 2363 |
| LLB2.10.8.9.11.N | 1200 | 4500 |
| LLB2.10.8.10.1.N | 500 | 1800 |
| LLB2.10.8.10.3.N | 490 | 2000 |
| LLB2.10.8.10.8.N | 650 | 2200 |
| LLB2-10-8-d1 MV | 250 | 1200 |
| LLB2-10-8-d2 MV | 430 | 2000 |
| LLB2-10-8-d3 MV | 470 | 2200 |
| LLB2-10-8-d4 MV | 690 | 2300 |
| LLB2-10-8-d5 MV | 1400 | 5000 |
| LLB2-10-8-d6 MV | 1200 | 4300 |
| LLB2-10-8-d1 BV | 280 | 1500 |
| LLB2-10-8-d2 BV | 490 | 2300 |
| LLB2-10-8-d3 BV | 490 | 2600 |
| LLB2-10-8-d4 BV | 670 | 2600 |
| LLB2-10-8-d5 BV | 1470 | 5500 |
| LLB2-10-8-d6 BV | 1480 | 5300 |
| LLB2-10-8-d7 BV | 410 | 2000 |
| LLB2-10-8-d8 BV | 820 | 3500 |
| LLB2-10-8-d9 BV | 840 | 4200 |
| LLB2-10-8-d10 BV | 1400 | 4200 |
| LLB2-10-8-d11 BV | 2400 | 7200 |
| LLB2-10-8-d12 BV | 2100 | 5968 |
| LLB2-10-8-d13 BV | 650 | 2600 |
| LLB2-10-8-d14 BV | 960 | 4054 |
| LLB2-10-8-d15 BV | 1000 | 4600 |
| LLB2-10-8-d16 BV | 650 | 2500 |
| LLB2-10-8-d17 BV | 960 | 4100 |
| LLB2-10-8-d18 BV | 880 | 3010 |

TABLE 9B
Affinity measurement for
additional LLB2 variants

| | Human | |
|---|---|---|
| | CD98hc | |
| | (uM) | |
| LLB2-10-8.d19 | Not yet tested | |
| LLB2-10-8.d20 | 2.3 | μM |
| LLB2-10-8.d21 | 2.0 | μM |
| LLB2-10-8.d22 | 5.2 | μM |
| LLB2-10-8.d23 | 1.8 | μM |
| LLB2-10-8.d24 | 2.6 | μM |
| LLB2-10-8.d25 | 670 | nM |
| LLB2-10-8.d26 | No binding | |
| LLB2-10-8.d27 | No binding | |
| LLB2-10-8.d28 | 4.2 | μM |
| LLB2-10-8.d29 | 1.3 | μM |
| LLB2-10-8.d30 | 1.0 | μM |
| LLB2-10-8.d31 | 460 | nM |
| LLB2-10-8.d32 | 3.1 | μM |

TABLE A2
Hits for LLB2-AM1 and LLB-AM2 engineering rounds

| | 369 | 370 | 371 | 372 | 373 | 374 | 375 | 376 | 377 | 378 | 379 | 380 | 381 | 382 | 383 | 384 | 385 | 386 |
|---|---|---|---|---|---|---|---|---|---|---|---|---|---|---|---|---|---|---|
| WT | V | K | G | F | Y | P | S | D | I | A | V | E | W | E | S | N | G | Q |
| Fc | | | | | | | | | | | | | | | | | | |
| LLB2- | . | . | . | . | . | . | . | . | . | . | . | L | . | N | . | R | F | S |
| 1 | | | | | | | | | | | | | | | | | | |
| LLB2- | . | . | . | . | . | . | . | . | . | . | . | I | . | N | . | R | F | S |
| 2 | | | | | | | | | | | | | | | | | | |
| LLB2- | . | . | . | . | . | . | . | . | . | . | . | M | . | N | . | R | F | S |
| 3 | | | | | | | | | | | | | | | | | | |
| LLB2- | . | . | . | . | . | . | . | . | . | . | . | L | . | S | . | R | F | S |
| 4 | | | | | | | | | | | | | | | | | | |
| LLB2- | . | . | . | . | . | . | . | . | . | . | . | L | . | N | . | K | F | S |
| 5 | | | | | | | | | | | | | | | | | | |
| LLB2- | . | . | . | . | . | . | . | . | . | . | . | L | . | N | . | Q | F | S |
| 6 | | | | | | | | | | | | | | | | | | |
| LLB2- | . | . | . | . | . | . | . | . | . | . | . | L | . | N | . | R | Y | S |
| 7 | | | | | | | | | | | | | | | | | | |
| LLB2- | . | . | . | . | . | . | . | . | . | . | . | L | . | N | . | R | F | E |
| 8 | | | | | | | | | | | | | | | | | | |
| LLB2- | . | . | . | . | . | . | . | . | . | . | . | L | . | N | . | R | F | . |
| 9 | | | | | | | | | | | | | | | | | | |
| LLB2- | . | . | . | . | . | . | . | . | . | . | . | L | . | N | . | R | F | V |
| 10 | | | | | | | | | | | | | | | | | | |
| LLB2- | . | . | . | . | . | . | . | . | . | . | . | L | . | N | . | R | F | S |
| 11 | | | | | | | | | | | | | | | | | | |
| LLB2- | . | . | . | . | . | . | . | . | . | . | . | L | . | N | . | R | F | S |
| 12 | | | | | | | | | | | | | | | | | | |
| LLB2- | . | . | . | . | . | . | . | . | . | . | . | L | . | N | . | Q | F | L |
| 13 | | | | | | | | | | | | | | | | | | |
| | 387 | 388 | 389 | 390 | 391 | 392 | 418 | 419 | 420 | 421 | 422 | 423 | 424 | 425 | 426 | 427 | 428 | 429 |
| WT | P | E | N | N | Y | K | Q | Q | G | N | V | F | S | C | S | V | M | H |
| Fc | | | | | | | | | | | | | | | | | | |
| LLB2- | L | . | . | . | . | . | . | . | . | . | I | . | A | . | N | . | L | . |
| 1 | | | | | | | | | | | | | | | | | | |
| LLB2- | L | . | . | . | . | . | . | . | . | . | I | . | A | . | N | . | L | . |
| 2 | | | | | | | | | | | | | | | | | | |
| LLB2- | L | . | . | . | . | . | . | . | . | . | I | . | A | . | N | . | L | . |
| 3 | | | | | | | | | | | | | | | | | | |
| LLB2- | L | . | . | . | . | . | . | . | . | . | I | . | A | . | N | . | L | . |
| 4 | | | | | | | | | | | | | | | | | | |
| LLB2- | L | . | . | . | . | . | . | . | . | . | I | . | A | . | N | . | L | . |
| 5 | | | | | | | | | | | | | | | | | | |
| LLB2- | L | . | . | . | . | . | . | . | . | . | I | . | A | . | N | . | L | . |
| 6 | | | | | | | | | | | | | | | | | | |
| LLB2- | L | . | . | . | . | . | . | . | . | . | I | . | A | . | N | . | L | . |
| 7 | | | | | | | | | | | | | | | | | | |
| LLB2- | L | . | . | . | . | . | . | . | . | . | I | . | A | . | N | . | L | . |
| 8 | | | | | | | | | | | | | | | | | | |
| LLB2- | L | . | . | . | . | . | . | . | . | . | I | . | A | . | N | . | L | . |
| 9 | | | | | | | | | | | | | | | | | | |
| LLB2- | L | . | . | . | . | . | . | . | . | . | I | . | A | . | N | . | L | . |
| 10 | | | | | | | | | | | | | | | | | | |
| LLB2- | L | . | . | . | . | . | . | . | . | . | I | . | A | . | N | . | . | . |
| 11 | | | | | | | | | | | | | | | | | | |
| LLB2- | L | . | . | . | . | . | . | . | . | . | I | . | A | . | N | . | L | . |
| 12 | | | | | | | | | | | | | | | | | | |
| LLB2- | L | . | . | . | . | . | . | . | . | . | I | . | A | . | N | . | L | . |
| 13 | | | | | | | | | | | | | | | | | | |
| | 430 | 431 | 432 | 433 | 434 | 435 | 436 | 437 | 438 | 439 | 440 | 441 | 442 | 443 | 444 | 445 | 446 | 447 |
| WT | E | A | L | H | N | H | Y | T | Q | K | S | L | S | L | S | P | G | K |
| Fc | | | | | | | | | | | | | | | | | | |
| LLB2- | . | . | . | . | . | . | . | . | F | . | N | | | | | | | |
| 1 | | | | | | | | | | | | | | | | | | |
| LLB2- | . | . | . | . | . | . | . | . | F | . | N | | | | | | | |
| 2 | | | | | | | | | | | | | | | | | | |
| LLB2- | . | . | . | . | . | . | . | . | F | . | N | | | | | | | |
| 3 | | | | | | | | | | | | | | | | | | |
| LLB2- | . | . | . | . | . | . | . | . | F | . | N | | | | | | | |
| 4 | | | | | | | | | | | | | | | | | | |
| LLB2- | . | . | . | . | . | . | . | . | F | . | N | | | | | | | |
| 5 | | | | | | | | | | | | | | | | | | |
| LLB2- | . | . | . | . | . | . | . | . | F | . | N | | | | | | | |
| 6 | | | | | | | | | | | | | | | | | | |
| LLB2- | . | . | . | . | . | . | . | . | F | . | N | | | | | | | |
| 7 | | | | | | | | | | | | | | | | | | |
| LLB2- | . | . | . | . | . | . | . | . | F | . | N | | | | | | | |
| 8 | | | | | | | | | | | | | | | | | | |
| LLB2- | . | . | . | . | . | . | . | . | F | . | N | | | | | | | |
| 9 | | | | | | | | | | | | | | | | | | |
| LLB2- | . | . | . | . | . | . | . | . | F | . | N | | | | | | | |
| 10 | | | | | | | | | | | | | | | | | | |
| LLB2- | . | . | . | . | . | . | . | . | F | . | N | | | | | | | |
| 11 | | | | | | | | | | | | | | | | | | |
| LLB2- | . | . | . | . | S | . | . | . | F | . | N | | | | | | | |
| 12 | | | | | | | | | | | | | | | | | | |
| LLB2- | . | . | . | . | . | . | . | . | F | . | N | | | | | | | |
| 13 | | | | | | | | | | | | | | | | | | |

TABLE A3
Hits for LLB2-AM3 and LLB-AM4 engineering rounds

| | 369 | 370 | 371 | 372 | 373 | 374 | 375 | 376 | 377 | 378 | 379 | 380 | 381 | 382 | 383 | 384 | 385 | 386 |
|---|---|---|---|---|---|---|---|---|---|---|---|---|---|---|---|---|---|---|
| WT | V | K | G | F | Y | P | S | D | I | A | V | E | W | E | S | N | G | Q |
| Fc | | | | | | | | | | | | | | | | | | |
| LLB2- | . | . | . | . | . | . | . | . | . | S | . | L | . | N | . | R | F | A |
| 17 | | | | | | | | | | | | | | | | | | |
| LLB2- | . | . | . | . | . | . | . | . | . | V | . | L | . | N | . | R | F | A |
| 18 | | | | | | | | | | | | | | | | | | |
| LLB2- | . | . | . | . | . | . | . | . | . | V | . | L | . | N | . | R | F | S |
| 19 | | | | | | | | | | | | | | | | | | |
| LLB2- | . | . | . | . | . | . | . | . | . | V | . | L | . | N | . | R | F | V |
| 20 | | | | | | | | | | | | | | | | | | |
| LLB2- | . | . | . | . | . | . | . | . | . | D | . | L | . | N | . | R | F | L |
| 21 | | | | | | | | | | | | | | | | | | |
| LLB2- | . | . | . | . | . | . | . | . | . | V | . | L | . | N | . | R | F | V |
| 22 | | | | | | | | | | | | | | | | | | |
| LLB2- | . | . | . | . | . | . | . | . | . | V | . | L | . | N | . | R | F | V |
| 23 | | | | | | | | | | | | | | | | | | |
| LLB2- | . | . | . | . | . | . | . | . | . | E | . | L | . | N | T | R | F | V |
| 24 | | | | | | | | | | | | | | | | | | |
| LLB2- | . | . | . | . | . | . | . | . | . | Y | . | L | . | N | . | R | F | L |
| 25 | | | | | | | | | | | | | | | | | | |
| LLB2- | . | . | . | . | . | . | . | . | . | . | . | L | . | L | F | H | F | V |
| 26 | | | | | | | | | | | | | | | | | | |
| LLB2- | . | . | . | . | . | . | . | . | . | . | . | L | . | M | N | H | F | T |
| 27 | | | | | | | | | | | | | | | | | | |
| LLB2- | . | . | . | . | . | . | . | . | . | . | . | L | . | P | P | I | F | T |
| 28 | | | | | | | | | | | | | | | | | | |
| LLB2- | . | . | . | . | . | . | . | . | . | . | . | L | . | Y | D | L | F | V |
| 29 | | | | | | | | | | | | | | | | | | |
| LLB2- | . | . | . | . | . | . | . | . | . | . | . | L | . | N | . | Q | F | L |
| 30 | | | | | | | | | | | | | | | | | | |
| LLB2- | . | . | . | . | . | . | . | . | . | . | . | L | . | N | . | Q | F | V |
| 31 | | | | | | | | | | | | | | | | | | |
| LLB2- | . | . | . | . | . | . | . | . | . | . | . | L | . | L | D | F | F | S |
| 32 | | | | | | | | | | | | | | | | | | |
| LLB2- | . | . | . | . | . | . | . | . | . | . | . | L | . | N | L | F | F | L |
| 33 | | | | | | | | | | | | | | | | | | |
| LLB2- | . | . | . | . | . | . | . | . | . | . | . | L | . | T | Q | F | F | A |
| 34 | | | | | | | | | | | | | | | | | | |
| LLB2- | . | . | . | . | . | . | . | . | . | . | . | L | . | A | P | Y | F | Y |
| 35 | | | | | | | | | | | | | | | | | | |
| LLB2- | . | . | . | . | . | . | . | . | . | . | . | L | . | K | H | V | F | Y |
| 36 | | | | | | | | | | | | | | | | | | |
| LLB2- | . | . | . | . | . | . | . | . | . | . | . | L | . | N | . | Q | F | H |
| 37 | | | | | | | | | | | | | | | | | | |
| | 387 | 388 | 389 | 390 | 391 | 392 | 418 | 419 | 420 | 421 | 422 | 423 | 424 | 425 | 426 | 427 | 428 | 429 |
| WT | P | E | N | N | Y | K | Q | Q | G | N | V | F | S | C | S | V | M | H |
| Fc | | | | | | | | | | | | | | | | | | |
| LLB2- | L | . | D | . | T | . | . | . | . | E | I | . | A | . | N | . | L | . |
| 17 | | | | | | | | | | | | | | | | | | |
| LLB2- | L | . | Q | . | V | . | . | . | . | Q | I | . | A | . | N | . | L | . |
| 18 | | | | | | | | | | | | | | | | | | |
| LLB2- | L | . | A | . | V | . | . | . | . | Q | I | . | A | . | N | . | L | . |
| 19 | | | | | | | | | | | | | | | | | | |
| LLB2- | L | . | D | . | V | . | . | . | . | A | I | . | A | . | N | . | L | . |
| 20 | | | | | | | | | | | | | | | | | | |
| LLB2- | L | . | T | . | T | . | . | . | . | E | I | . | A | . | N | . | L | . |
| 21 | | | | | | | | | | | | | | | | | | |
| LLB2- | L | . | H | . | A | . | . | . | . | . | I | . | A | . | N | . | L | . |
| 22 | | | | | | | | | | | | | | | | | | |
| LLB2- | L | . | Q | . | V | . | . | . | . | E | I | . | A | . | N | . | L | . |
| 23 | | | | | | | | | | | | | | | | | | |
| LLB2- | L | . | T | . | A | . | . | . | . | E | I | . | A | . | N | . | L | . |
| 24 | | | | | | | | | | | | | | | | | | |
| LLB2- | L | . | V | . | . | . | . | . | . | E | I | . | A | . | N | . | L | . |
| 25 | | | | | | | | | | | | | | | | | | |
| LLB2- | L | . | . | . | . | . | . | . | . | . | I | . | A | . | N | . | T | . |
| 26 | | | | | | | | | | | | | | | | | | |
| LLB2- | L | . | . | . | . | . | . | . | . | . | I | . | A | . | N | . | . | . |
| 27 | | | | | | | | | | | | | | | | | | |
| LLB2- | L | . | . | . | . | . | . | . | . | . | I | . | A | . | N | . | P | . |
| 28 | | | | | | | | | | | | | | | | | | |
| LLB2- | L | . | . | . | . | . | . | . | . | . | I | . | A | . | N | . | L | . |
| 29 | | | | | | | | | | | | | | | | | | |
| LLB2- | L | . | . | . | . | . | . | . | . | . | I | . | A | . | N | . | Y | . |
| 30 | | | | | | | | | | | | | | | | | | |
| LLB2- | L | . | . | . | . | . | . | . | . | . | I | . | A | . | N | . | Y | . |
| 31 | | | | | | | | | | | | | | | | | | |
| LLB2- | L | . | . | . | . | . | . | . | . | . | I | . | A | . | N | . | F | . |
| 32 | | | | | | | | | | | | | | | | | | |
| LLB2- | L | . | . | . | . | . | . | . | . | . | I | . | A | . | N | . | . | . |
| 33 | | | | | | | | | | | | | | | | | | |
| LLB2- | L | . | . | . | . | . | . | . | . | . | I | . | A | . | N | . | I | . |
| 34 | | | | | | | | | | | | | | | | | | |
| LLB2- | L | . | . | . | . | . | . | . | . | . | I | . | A | . | N | . | A | . |
| 35 | | | | | | | | | | | | | | | | | | |
| LLB2- | L | . | . | . | . | . | . | . | . | . | I | . | A | . | N | . | K | . |
| 36 | | | | | | | | | | | | | | | | | | |
| LLB2- | L | . | . | . | . | . | . | . | . | . | I | . | A | . | N | . | Y | . |
| 37 | | | | | | | | | | | | | | | | | | |
| | 430 | 431 | 432 | 433 | 434 | 435 | 436 | 437 | 438 | 439 | 440 | 441 | 442 | 443 | 444 | 445 | 446 | 447 |
| WT | E | A | L | H | N | H | Y | T | Q | K | S | L | S | L | S | P | G | K |
| Fc | | | | | | | | | | | | | | | | | | |
| LLB2- | . | . | . | . | . | . | . | . | F | . | N | . | A | . | . | . | . | . |
| 17 | | | | | | | | | | | | | | | | | | |
| LLB2- | . | . | . | . | . | . | L | . | F | . | N | . | V | . | . | . | . | . |
| 18 | | | | | | | | | | | | | | | | | | |
| LLB2- | . | . | . | . | . | . | V | . | F | . | N | . | V | . | . | . | . | . |
| 19 | | | | | | | | | | | | | | | | | | |
| LLB2- | . | . | . | . | . | . | H | . | F | . | N | . | V | . | . | . | . | . |
| 20 | | | | | | | | | | | | | | | | | | |
| LLB2- | . | . | . | . | . | . | . | . | F | . | N | . | K | . | . | . | . | . |
| 21 | | | | | | | | | | | | | | | | | | |
| LLB2- | . | . | . | . | . | . | . | . | F | . | N | . | M | . | . | . | . | . |
| 22 | | | | | | | | | | | | | | | | | | |
| LLB2- | . | . | . | . | . | . | . | . | F | . | N | . | V | . | . | . | . | . |
| 23 | | | | | | | | | | | | | | | | | | |
| LLB2- | . | . | . | . | . | . | F | . | F | . | N | . | A | . | . | . | . | . |
| 24 | | | | | | | | | | | | | | | | | | |
| LLB2- | . | . | . | . | . | . | P | . | F | . | N | . | V | . | . | . | . | . |
| 25 | | | | | | | | | | | | | | | | | | |
| LLB2- | . | . | . | . | . | . | . | . | F | . | L | . | F | . | . | . | . | . |
| 26 | | | | | | | | | | | | | | | | | | |
| LLB2- | . | . | . | . | . | . | . | . | F | . | P | . | P | . | . | . | . | . |
| 27 | | | | | | | | | | | | | | | | | | |
| LLB2- | . | . | . | . | . | . | . | . | F | . | L | . | A | . | . | . | . | . |
| 28 | | | | | | | | | | | | | | | | | | |
| LLB2- | . | . | . | . | . | . | . | . | F | . | E | P | F | . | . | . | . | . |
| 29 | | | | | | | | | | | | | | | | | | |
| LLB2- | . | . | . | . | . | . | . | . | F | . | N | . | Q | . | . | . | . | . |
| 30 | | | | | | | | | | | | | | | | | | |
| LLB2- | . | . | . | . | . | . | . | . | F | . | N | . | A | . | . | . | . | . |
| 31 | | | | | | | | | | | | | | | | | | |
| LLB2- | . | . | . | . | . | . | . | . | F | . | V | . | A | . | . | . | . | . |
| 32 | | | | | | | | | | | | | | | | | | |
| LLB2- | . | . | . | . | . | . | . | . | F | . | A | P | V | . | . | . | . | . |
| 33 | | | | | | | | | | | | | | | | | | |
| LLB2- | . | . | . | . | . | . | . | . | F | . | V | P | Q | . | . | . | . | . |
| 34 | | | | | | | | | | | | | | | | | | |
| LLB2- | . | . | . | . | . | . | . | . | F | . | I | . | P | . | . | . | . | . |
| 35 | | | | | | | | | | | | | | | | | | |
| LLB2- | . | . | . | . | . | . | . | . | F | . | P | P | M | . | . | . | . | . |
| 36 | | | | | | | | | | | | | | | | | | |
| LLB2- | . | . | . | . | . | . | . | . | F | . | N | . | L | . | . | . | . | . |
| 37 | | | | | | | | | | | | | | | | | | |

TABLE A4
Hits for LLB2-10-X rational design engineering round

| | 369 | 370 | 371 | 372 | 373 | 374 | 375 | 376 | 377 | 378 | 379 | 380 | 381 | 382 | 383 | 384 | 385 | 386 |
|---|---|---|---|---|---|---|---|---|---|---|---|---|---|---|---|---|---|---|
| WT | V | K | G | F | Y | P | S | D | I | A | V | E | W | E | S | N | G | Q |
| Fc | | | | | | | | | | | | | | | | | | |
| LLB2- | . | . | . | . | . | . | . | . | . | . | . | L | . | N | . | R | F | V |
| 10-1 | | | | | | | | | | | | | | | | | | |
| LLB2- | . | . | . | . | . | . | . | . | . | . | . | L | . | N | . | R | F | V |
| 10-2 | | | | | | | | | | | | | | | | | | |
| LLB2- | . | . | . | . | . | . | . | . | . | . | . | L | . | N | . | R | F | V |
| 10-3 | | | | | | | | | | | | | | | | | | |
| LLB2- | . | . | . | . | . | . | . | . | . | V | . | L | . | N | . | R | F | V |
| 10-4 | | | | | | | | | | | | | | | | | | |
| LLB2- | . | . | . | . | . | . | . | . | . | . | . | L | . | N | . | R | F | V |
| 10-5 | | | | | | | | | | | | | | | | | | |
| LLB2- | . | . | . | . | . | . | . | . | . | . | . | L | . | N | . | R | F | V |
| 10-6 | | | | | | | | | | | | | | | | | | |
| LLB2- | . | . | . | . | . | . | . | . | . | . | . | L | . | N | . | R | F | V |
| 10-7 | | | | | | | | | | | | | | | | | | |
| LLB2- | . | . | . | . | . | . | . | . | . | . | . | L | . | N | . | R | F | V |
| 10-8 | | | | | | | | | | | | | | | | | | |
| LLB2- | . | . | . | . | . | . | . | . | . | V | . | L | . | N | . | R | F | V |
| 10-9 | | | | | | | | | | | | | | | | | | |
| LLB2- | . | . | . | . | . | . | . | . | . | . | . | L | . | N | . | Q | F | V |
| 10-10 | | | | | | | | | | | | | | | | | | |
| LLB2- | . | . | . | . | . | . | . | . | . | . | . | L | . | N | . | R | F | L |
| 10-11 | | | | | | | | | | | | | | | | | | |
| LLB2- | . | . | . | . | . | . | . | . | . | . | . | L | . | N | . | R | F | V |
| 10-12 | | | | | | | | | | | | | | | | | | |
| LLB2- | . | . | . | . | . | . | . | . | . | . | . | L | . | N | . | Q | F | L |
| 10-13 | | | | | | | | | | | | | | | | | | |
| LLB2- | . | . | . | . | . | . | . | . | . | . | . | L | . | N | . | R | F | V |
| 10-5 | | | | | | | | | | | | | | | | | | |
| Mono | | | | | | | | | | | | | | | | | | |
| LLB2- | . | . | . | . | . | . | . | . | . | . | . | L | . | N | . | R | F | V |
| 10-6 | | | | | | | | | | | | | | | | | | |
| Mono | | | | | | | | | | | | | | | | | | |
| LLB2- | . | . | . | . | . | . | . | . | . | . | . | L | . | N | . | R | F | V |
| 10-8 | | | | | | | | | | | | | | | | | | |
| Mono | | | | | | | | | | | | | | | | | | |
| | 387 | 388 | 389 | 390 | 391 | 392 | 418 | 419 | 420 | 421 | 422 | 423 | 424 | 425 | 426 | 427 | 428 | 429 |
| WT | P | E | N | N | Y | K | Q | Q | G | N | V | F | S | C | S | V | M | H |
| Fc | | | | | | | | | | | | | | | | | | |
| LLB2- | L | . | . | . | . | . | . | . | . | . | I | . | A | . | N | . | L | . |
| 10-1 | | | | | | | | | | | | | | | | | | |
| LLB2- | L | . | . | . | . | . | . | . | . | . | I | . | A | . | N | . | L | . |
| 10-2 | | | | | | | | | | | | | | | | | | |
| LLB2- | L | . | . | . | . | . | . | . | . | . | I | . | A | . | N | . | L | . |
| 10-3 | | | | | | | | | | | | | | | | | | |
| LLB2- | L | . | . | . | . | . | . | . | . | . | I | . | A | . | N | . | L | . |
| 10-4 | | | | | | | | | | | | | | | | | | |
| LLB2- | L | . | . | . | . | . | . | . | . | E | I | . | A | . | N | . | L | . |
| 10-5 | | | | | | | | | | | | | | | | | | |
| LLB2- | L | . | . | . | . | . | . | . | . | . | I | . | A | . | N | . | Y | . |
| 10-6 | | | | | | | | | | | | | | | | | | |
| LLB2- | L | . | . | . | . | . | . | . | . | . | I | . | A | . | N | . | L | . |
| 10-7 | | | | | | | | | | | | | | | | | | |
| LLB2- | L | . | . | . | . | . | . | . | . | E | I | . | A | . | N | . | Y | . |
| 10-8 | | | | | | | | | | | | | | | | | | |
| LLB2- | L | . | . | . | . | . | . | . | . | E | I | . | A | . | N | . | Y | . |
| 10-9 | | | | | | | | | | | | | | | | | | |
| LLB2- | L | . | . | . | . | . | . | . | . | E | I | . | A | . | N | . | Y | . |
| 10-10 | | | | | | | | | | | | | | | | | | |
| LLB2- | L | . | . | . | . | . | . | . | . | E | I | . | A | . | N | . | Y | . |
| 10-11 | | | | | | | | | | | | | | | | | | |
| LLB2- | L | . | . | . | . | . | . | . | . | E | I | . | A | . | N | . | Y | . |
| 10-12 | | | | | | | | | | | | | | | | | | |
| LLB2- | L | . | . | . | . | . | . | . | . | E | I | . | A | . | N | . | Y | . |
| 10-13 | | | | | | | | | | | | | | | | | | |
| LLB2- | L | . | . | . | . | . | . | . | . | E | I | . | A | . | N | . | L | . |
| 10-5 | | | | | | | | | | | | | | | | | | |
| Mono | | | | | | | | | | | | | | | | | | |
| LLB2- | L | . | . | . | . | . | . | . | . | . | I | . | A | . | N | . | Y | . |
| 10-6 | | | | | | | | | | | | | | | | | | |
| Mono | | | | | | | | | | | | | | | | | | |
| LLB2- | L | . | . | . | . | . | . | . | . | E | I | . | A | . | N | . | Y | . |
| 10-8 | | | | | | | | | | | | | | | | | | |
| Mono | | | | | | | | | | | | | | | | | | |
| | 430 | 431 | 432 | 433 | 434 | 435 | 436 | 437 | 438 | 439 | 440 | 441 | 442 | 443 | 444 | 445 | 446 | 447 |
| WT | E | A | L | H | N | H | Y | T | Q | K | S | L | S | L | S | P | G | K |
| Fc | | | | | | | | | | | | | | | | | | |
| LLB2- | . | . | . | . | . | . | . | . | F | . | N | . | A | . | . | . | . | . |
| 10-1 | | | | | | | | | | | | | | | | | | |
| LLB2- | . | . | . | . | . | . | . | . | F | . | N | . | Y | . | . | . | . | . |
| 10-2 | | | | | | | | | | | | | | | | | | |
| LLB2- | . | . | . | . | . | . | . | . | F | . | N | P | . | . | . | . | . | . |
| 10-3 | | | | | | | | | | | | | | | | | | |
| LLB2- | . | . | . | . | . | . | . | . | F | . | N | . | A | . | . | . | . | . |
| 10-4 | | | | | | | | | | | | | | | | | | |
| LLB2- | . | . | . | . | . | . | . | . | F | . | N | . | A | . | . | . | . | . |
| 10-5 | | | | | | | | | | | | | | | | | | |
| LLB2- | . | . | . | . | . | . | . | . | F | . | N | . | A | . | . | . | . | . |
| 10-6 | | | | | | | | | | | | | | | | | | |
| LLB2- | . | . | . | . | S | . | . | . | F | . | N | . | A | . | . | . | . | . |
| 10-7 | | | | | | | | | | | | | | | | | | |
| LLB2- | . | . | . | . | . | . | . | . | F | . | N | . | A | . | . | . | . | . |
| 10-8 | | | | | | | | | | | | | | | | | | |
| LLB2- | . | . | . | . | . | . | . | . | F | . | N | . | A | . | . | . | . | . |
| 10-9 | | | | | | | | | | | | | | | | | | |
| LLB2- | . | . | . | . | . | . | . | . | F | . | N | . | A | . | . | . | . | . |
| 10-10 | | | | | | | | | | | | | | | | | | |
| LLB2- | . | . | . | . | . | . | . | . | F | . | N | . | A | . | . | . | . | . |
| 10-11 | | | | | | | | | | | | | | | | | | |
| LLB2- | . | . | . | . | . | . | . | . | F | . | N | . | Q | . | . | . | . | . |
| 10-12 | | | | | | | | | | | | | | | | | | |
| LLB2- | . | . | . | . | . | . | . | . | F | . | N | . | Q | . | . | . | . | . |
| 10-13 | | | | | | | | | | | | | | | | | | |
| LLB2- | . | . | . | . | . | . | . | . | F | . | N | . | A | . | . | . | . | . |
| 10-5 | | | | | | | | | | | | | | | | | | |
| Mono | | | | | | | | | | | | | | | | | | |
| LLB2- | . | . | . | . | . | . | . | . | F | . | N | . | A | . | . | . | . | . |
| 10-6 | | | | | | | | | | | | | | | | | | |
| Mono | | | | | | | | | | | | | | | | | | |
| LLB2- | . | . | . | . | . | . | . | . | F | . | N | . | A | . | . | . | . | . |
| 10-8 | | | | | | | | | | | | | | | | | | |
| Mono | | | | | | | | | | | | | | | | | | |

TABLE A5
Hits for soft mutagenesis of LLB2 by yeast display

| | 369 | 370 | 371 | 372 | 373 | 374 | 375 | 376 | 377 | 378 | 379 | 380 | 381 | 382 | 383 | 384 | 385 | 386 |
|---|---|---|---|---|---|---|---|---|---|---|---|---|---|---|---|---|---|---|
| WT | V | K | G | F | Y | P | S | D | I | A | V | E | W | E | S | N | G | Q |
| Fc | | | | | | | | | | | | | | | | | | |
| LLB2. | . | . | . | . | . | . | . | . | . | . | . | A | . | N | . | K | F | E |
| 33.2 | | | | | | | | | | | | | | | | | | |
| LLB2. | . | . | . | . | . | . | . | . | . | . | . | . | . | S | . | Q | Y | E |
| 33.3 | | | | | | | | | | | | | | | | | | |
| LLB2. | . | . | . | . | . | . | . | . | . | . | . | A | . | N | . | K | F | R |
| 33.8 | | | | | | | | | | | | | | | | | | |
| LLB2. | . | . | . | . | . | . | . | . | . | . | . | Q | . | S | . | Q | Y | E |
| 33.5 | | | | | | | | | | | | | | | | | | |
| LLB2. | . | . | . | . | . | . | . | . | . | . | . | M | . | N | . | Q | Y | E |
| 43.5 | | | | | | | | | | | | | | | | | | |
| LLB2. | . | . | . | . | . | . | . | . | . | . | . | V | . | N | . | Q | Y | E |
| C.1 | | | | | | | | | | | | | | | | | | |
| LLB2. | . | . | . | . | . | . | . | . | . | . | . | V | . | N | . | Q | Y | E |
| C.3 | | | | | | | | | | | | | | | | | | |
| LLB2. | . | . | . | . | . | . | . | . | . | . | . | A | . | N | . | R | F | E |
| C.4 | | | | | | | | | | | | | | | | | | |
| LLB2. | . | . | . | . | . | . | . | . | . | . | . | K | . | N | . | R | F | . |
| C.15 | | | | | | | | | | | | | | | | | | |
| | 387 | 388 | 389 | 390 | 391 | 392 | 418 | 419 | 420 | 421 | 422 | 423 | 424 | 425 | 426 | 427 | 428 | 429 |
| WT | P | E | N | N | Y | K | Q | Q | G | N | V | F | S | C | S | V | M | H |
| Fc | | | | | | | | | | | | | | | | | | |
| LLB2. | L | . | . | . | . | . | . | . | . | . | L | . | A | . | N | . | W | . |
| 33.2 | | | | | | | | | | | | | | | | | | |
| LLB2. | L | . | . | . | . | . | . | . | . | . | M | . | A | . | N | . | W | . |
| 33.3 | | | | | | | | | | | | | | | | | | |
| LLB2. | L | . | . | . | . | . | . | . | . | . | L | . | A | . | N | . | W | . |
| 33.8 | | | | | | | | | | | | | | | | | | |
| LLB2. | L | . | . | . | . | . | . | . | . | . | . | . | A | . | N | . | W | . |
| 33.5 | | | | | | | | | | | | | | | | | | |
| LLB2. | L | . | . | . | . | . | . | . | . | . | . | . | A | . | N | . | H | . |
| 43.5 | | | | | | | | | | | | | | | | | | |
| LLB2. | L | . | . | . | . | . | . | . | . | . | P | . | A | . | N | . | W | . |
| C.1 | | | | | | | | | | | | | | | | | | |
| LLB2. | L | . | . | . | . | . | . | . | . | . | . | . | A | . | N | . | W | . |
| C.3 | | | | | | | | | | | | | | | | | | |
| LLB2. | L | . | . | . | . | . | . | . | . | . | I | . | A | . | N | . | W | . |
| C.4 | | | | | | | | | | | | | | | | | | |
| LLB2. | L | . | . | . | . | . | . | . | . | . | L | . | A | . | N | . | W | . |
| C.15 | | | | | | | | | | | | | | | | | | |
| | 430 | 431 | 432 | 433 | 434 | 435 | 436 | 437 | 438 | 439 | 440 | 441 | 442 | 443 | 444 | 445 | 446 | 447 |
| WT | E | A | L | H | N | H | Y | T | Q | K | S | L | S | L | S | P | G | K |
| Fc | | | | | | | | | | | | | | | | | | |
| LLB2. | . | . | . | . | . | . | . | . | F | . | N | . | . | . | . | . | . | . |
| 33.2 | | | | | | | | | | | | | | | | | | |
| LLB2. | . | . | . | . | . | . | . | . | F | . | N | . | . | . | . | . | . | . |
| 33.3 | | | | | | | | | | | | | | | | | | |
| LLB2. | . | . | . | . | . | . | . | . | F | . | N | . | . | . | . | . | . | . |
| 33.8 | | | | | | | | | | | | | | | | | | |
| LLB2. | . | . | . | . | . | . | . | . | F | . | N | . | . | . | . | . | . | . |
| 33.5 | | | | | | | | | | | | | | | | | | |
| LLB2. | . | . | . | . | . | . | . | . | F | . | N | . | . | . | . | . | . | . |
| 43.5 | | | | | | | | | | | | | | | | | | |
| LLB2. | . | . | . | . | . | . | . | . | F | . | D | . | . | . | . | . | . | . |
| C.1 | | | | | | | | | | | | | | | | | | |
| LLB2. | . | . | . | . | . | . | . | . | F | . | N | . | . | . | . | . | . | . |
| C.3 | | | | | | | | | | | | | | | | | | |
| LLB2. | . | . | . | . | . | . | . | . | F | . | N | . | . | . | . | . | . | . |
| C.4 | | | | | | | | | | | | | | | | | | |
| LLB2. | . | . | . | . | . | . | . | . | F | . | N | . | . | . | . | . | . | . |
| C.15 | | | | | | | | | | | | | | | | | | |

TABLE A6
Hits for rational design engineering round for M428Y or E380 + M428Y

| | 369 | 370 | 371 | 372 | 373 | 374 | 375 | 376 | 377 | 378 | 379 | 380 | 381 | 382 | 383 | 384 | 385 | 386 |
|---|---|---|---|---|---|---|---|---|---|---|---|---|---|---|---|---|---|---|
| WT | V | K | G | F | Y | P | S | D | I | A | V | E | W | E | S | N | G | Q |
| Fc | | | | | | | | | | | | | | | | | | |
| LLB2- | . | . | . | . | . | . | . | . | . | . | . | . | . | N | . | R | F | V |
| 10-8-1 | | | | | | | | | | | | | | | | | | |
| LLB2- | . | . | . | . | . | . | . | . | . | . | . | L | . | N | . | Q | F | V |
| 10-8-3 | | | | | | | | | | | | | | | | | | |
| LLB2- | . | . | . | . | . | . | . | . | . | . | . | L | . | N | . | Q | F | L |
| 30 | | | | | | | | | | | | | | | | | | |
| LLB2- | . | . | . | . | . | . | . | . | . | . | . | L | . | N | . | Q | F | V |
| 31 | | | | | | | | | | | | | | | | | | |
| LLB2- | . | . | . | . | . | . | . | . | . | . | . | L | . | N | . | Q | F | H |
| 37 | | | | | | | | | | | | | | | | | | |
| LLBW- | . | . | . | . | . | . | . | . | . | V | . | L | . | N | . | R | F | V |
| 10-9 | | | | | | | | | | | | | | | | | | |
| LLB2- | . | . | . | . | . | . | . | . | . | . | . | L | . | N | . | R | F | L |
| 10-11 | | | | | | | | | | | | | | | | | | |
| LLB2- | . | . | . | . | . | . | . | . | . | . | . | L | . | N | . | R | F | V |
| 10-12 | | | | | | | | | | | | | | | | | | |
| LLB2- | . | . | . | . | . | . | . | . | . | . | . | L | . | N | . | Q | F | L |
| 10-13 | | | | | | | | | | | | | | | | | | |
| | 387 | 388 | 389 | 390 | 391 | 392 | 418 | 419 | 420 | 421 | 422 | 423 | 424 | 425 | 426 | 427 | 428 | 429 |
| WT | P | E | N | N | Y | K | Q | Q | G | N | V | F | S | C | S | V | M | H |
| Fc | | | | | | | | | | | | | | | | | | |
| LLB2- | L | . | . | . | . | . | . | . | . | E | I | . | A | . | N | . | . | . |
| 10-8-1 | | | | | | | | | | | | | | | | | | |
| LLB2- | L | . | . | . | . | . | . | . | . | E | I | . | A | . | N | . | Y | . |
| 10-8-3 | | | | | | | | | | | | | | | | | | |
| LLB2- | L | . | . | . | . | . | . | . | . | . | I | . | A | . | N | . | Y | . |
| 30 | | | | | | | | | | | | | | | | | | |
| LLB2- | L | . | . | . | . | . | . | . | . | . | I | . | A | . | N | . | Y | . |
| 31 | | | | | | | | | | | | | | | | | | |
| LLB2- | L | . | . | . | . | . | . | . | . | . | I | . | A | . | N | . | Y | . |
| 37 | | | | | | | | | | | | | | | | | | |
| LLBW- | L | . | . | . | . | . | . | . | . | E | I | . | A | . | N | . | Y | . |
| 10-9 | | | | | | | | | | | | | | | | | | |
| LLB2- | L | . | . | . | . | . | . | . | . | E | I | . | A | . | N | . | Y | . |
| 10-11 | | | | | | | | | | | | | | | | | | |
| LLB2- | L | . | . | . | . | . | . | . | . | E | I | . | A | . | N | . | Y | . |
| 10-12 | | | | | | | | | | | | | | | | | | |
| LLB2- | L | . | . | . | . | . | . | . | . | E | I | . | A | . | N | . | Y | . |
| 10-13 | | | | | | | | | | | | | | | | | | |
| | 430 | 431 | 432 | 433 | 434 | 435 | 436 | 437 | 438 | 439 | 440 | 441 | 442 | 443 | 444 | 445 | 446 | 447 |
| WT | E | A | L | H | N | H | Y | T | Q | K | S | L | S | L | S | P | G | K |
| Fc | | | | | | | | | | | | | | | | | | |
| LLB2- | . | . | . | . | . | . | . | . | F | . | N | . | A | . | . | . | . | . |
| 10-8-1 | | | | | | | | | | | | | | | | | | |
| LLB2- | . | . | . | . | . | . | . | . | F | . | N | . | A | . | . | . | . | . |
| 10-8-3 | | | | | | | | | | | | | | | | | | |
| LLB2- | . | . | . | . | . | . | . | . | F | . | N | . | Q | . | . | . | . | . |
| 30 | | | | | | | | | | | | | | | | | | |
| LLB2- | . | . | . | . | . | . | . | . | F | . | N | . | A | . | . | . | . | . |
| 31 | | | | | | | | | | | | | | | | | | |
| LLB2- | . | . | . | . | . | . | . | . | F | . | N | . | L | . | . | . | . | . |
| 37 | | | | | | | | | | | | | | | | | | |
| LLBW- | . | . | . | . | . | . | . | . | F | . | N | . | A | . | . | . | . | . |
| 10-9 | | | | | | | | | | | | | | | | | | |
| LLB2- | . | . | . | . | . | . | . | . | F | . | N | . | A | . | . | . | . | . |
| 10-11 | | | | | | | | | | | | | | | | | | |
| LLB2- | . | . | . | . | . | . | . | . | F | . | N | . | Q | . | . | . | . | . |
| 10-12 | | | | | | | | | | | | | | | | | | |
| LLB2- | . | . | . | . | . | . | . | . | F | . | N | . | Q | . | . | . | . | . |
| 10-13 | | | | | | | | | | | | | | | | | | |

TABLE A7
Patch library for LLB2 designs screened by yeast display

| | 376 | 377 | 378 | 379 | 380 | 381 | 382 | 383 | 384 | 385 | 386 | 387 | 388 | 389 | 390 | 390 | 392 |
|---|---|---|---|---|---|---|---|---|---|---|---|---|---|---|---|---|---|
| WT Fc | D | I | A | V | E | W | E | S | N | G | Q | P | E | N | N | Y | K |
| Library | | | | | L | | N | | R | F | V | L | | | | | |
| Scaffold | | | | | | | | | | | | | | | | | |
| Patch. 1 | | | | | | | NNK | | | | | | | | | | |
| Patch. 2 | | | | | | | | NNK | NNK | NNK | NNK | | | | | | |
| Patch. 3 | | | | | | | | NNK | | | NNK | NNK | | NNK | NNK | | |
| Patch. 4 | | | | | | | | | | | | | | | | | |
| Patch. 5 | | | | | | | NNK | | NNK | | | | | | | | |
| Patch. 6 | | | | | | | | NNK | | | | | | | | | |
| Patch. 7 | | | | | | | NNK | NNK | NNK | | | | | | | | |
| Patch. 8 | | | | | | | | | NNK | | NNK | | | | | | |
| Patch. 9 | | | | | | | | | | NNK | | | | | | | |
| Patch. 10 | | | | | | | | NNK | | | | | | | | | |
| LLB2. | . | . | . | . | L | . | N | . | Q | Y | L | L | . | . | . | . | . |
| 10.8.2.1 | | | | | | | | | | | | | | | | | |
| LLB2. | . | . | . | . | L | . | N | . | H | Y | V | L | . | . | . | . | . |
| 10.8.12.1 | | | | | | | | | | | | | | | | | |
| LLB2. | . | . | . | . | L | . | N | . | H | Y | I | L | . | . | . | . | . |
| 10.8.2.3 | | | | | | | | | | | | | | | | | |
| LLB2. | . | . | . | . | L | . | N | . | H | Y | L | L | . | . | . | . | . |
| 10.8.2.4 | | | | | | | | | | | | | | | | | |
| LLB2. | . | . | . | . | L | . | N | . | H | Y | E | L | . | . | . | . | . |
| 10.8.12.5 | | | | | | | | | | | | | | | | | |
| LLB2. | . | . | . | . | L | . | N | . | H | F | L | L | . | . | . | . | . |
| 10.8.2.15 | | | | | | | | | | | | | | | | | |
| LLB2. | . | . | . | . | L | . | N | . | R | F | V | L | . | . | . | . | . |
| 10.8.4.15 | | | | | | | | | | | | | | | | | |
| LLB2. | . | . | . | . | L | . | N | . | R | F | V | L | . | . | . | . | . |
| 10.8.4.8 | | | | | | | | | | | | | | | | | |
| LLB2. | . | . | . | . | L | . | N | . | R | F | V | L | . | . | . | . | . |
| 10.8.4.12 | | | | | | | | | | | | | | | | | |
| LLB2. | . | . | . | . | L | . | N | . | R | F | V | L | . | . | . | . | . |
| 10.8.14.3 | | | | | | | | | | | | | | | | | |
| LLB2. | . | . | . | . | L | . | N | . | R | F | V | L | . | . | . | . | . |
| 10.8.14.4 | | | | | | | | | | | | | | | | | |
| LLB2. | . | . | . | . | L | . | N | . | R | F | V | L | . | . | . | . | . |
| 10.8.14.12 | | | | | | | | | | | | | | | | | |
| LLB2. | . | . | . | . | L | . | N | . | H | F | V | L | . | . | . | . | . |
| 10.8.5.1 | | | | | | | | | | | | | | | | | |
| LLB2. | . | . | . | . | L | . | N | . | H | F | V | L | . | . | . | . | . |
| 10.8.5. | | | | | | | | | | | | | | | | | |
| 3star | | | | | | | | | | | | | | | | | |
| LLB2. | . | . | . | . | L | . | N | . | H | F | L | L | . | . | . | . | . |
| 10.8.18.5 | | | | | | | | | | | | | | | | | |
| LLB2. | . | . | . | . | L | . | N | . | H | F | V | L | . | . | . | . | . |
| 10.8.18.14 | | | | | | | | | | | | | | | | | |
| LLB2. | . | . | . | . | L | . | N | . | R | F | V | L | . | . | . | . | . |
| 10.8.9.11 | | | | | | | | | | | | | | | | | |
| LLB2. | . | . | . | . | L | . | N | . | R | F | V | L | . | . | . | . | . |
| 10.8.10.1 | | | | | | | | | | | | | | | | | |
| LLB2. | . | . | . | . | L | . | N | . | R | F | V | L | . | . | . | . | . |
| 10.8.10.3 | | | | | | | | | | | | | | | | | |
| LLB2. | . | . | . | . | L | . | N | . | R | F | V | L | . | . | . | . | . |
| 10.8.10.8 | | | | | | | | | | | | | | | | | |
| LLB2. | . | . | . | . | L | . | N | . | H | Y | L | L | . | . | . | . | . |
| 10.8.21 | | | | | | | | | | | | | | | | | |
| LLB2. | . | . | . | . | L | . | N | . | H | Y | L | L | . | . | . | . | . |
| 10.8.22 | | | | | | | | | | | | | | | | | |
| LLB2. | . | . | . | . | L | . | N | . | H | Y | L | L | . | . | . | . | . |
| 10.8.23 | | | | | | | | | | | | | | | | | |
| LLB2. | . | . | . | . | L | . | N | . | H | Y | L | L | . | . | . | . | . |
| 10.8.24 | | | | | | | | | | | | | | | | | |
| | 418 | 419 | 420 | 421 | 422 | 423 | 424 | 425 | 426 | 427 | 428 | 429 | 430 | 431 | 432 | 433 | 434 |
| WT Fc | Q | Q | G | N | V | F | S | C | S | V | M | H | E | A | L | H | N |
| Library | | | | E | I | | A | | N | | Y | | | | | | |
| Scaffold | | | | | | | | | | | | | | | | | |
| Patch. 1 | | | | NNK | NNK | | | | | | | | | | | | |
| Patch. 2 | | | | | | | | | | | | | | | | | |
| Patch. 3 | | | | | | | | | | | | | | | | | |
| Patch. 4 | | | | | | | | | | | | | | | | | |
| Patch. 5 | | | | | | | NNK | | NNK | | | | | | | | |
| Patch. 6 | | | | NNK | | | | | | | | | | | | | |
| Patch. 7 | | | | | | | NNK | | | | | | | | | | |
| Patch. 8 | | | | | NNK | | | | | | | | | | | | |
| Patch. 9 | | | | | NNK | | | | | | | | | | | | |
| Patch. 10 | | | | | | | | | NNK | | | | | | | | |
| LLB2. | . | . | . | E | I | . | A | . | N | . | Y | . | . | . | . | . | . |
| 10.8.2.1 | | | | | | | | | | | | | | | | | |
| LLB2. | . | . | . | E | I | . | A | . | N | . | Y | . | . | . | . | . | . |
| 10.8.12.1 | | | | | | | | | | | | | | | | | |
| LLB2. | . | . | . | E | I | . | A | . | N | . | Y | . | . | . | . | . | . |
| 10.8.2.3 | | | | | | | | | | | | | | | | | |
| LLB2. | . | . | . | E | I | . | A | . | N | . | Y | . | . | . | . | . | . |
| 10.8.2.4 | | | | | | | | | | | | | | | | | |
| LLB2. | . | . | . | E | I | . | A | . | N | . | Y | . | . | . | . | . | . |
| 10.8.12.5 | | | | | | | | | | | | | | | | | |
| LLB2. | . | . | . | E | I | . | A | . | N | . | Y | . | . | . | . | . | . |
| 10.8.2.15 | | | | | | | | | | | | | | | | | |
| LLB2. | . | . | . | E | I | . | A | . | N | . | Y | . | . | . | . | . | . |
| 10.8.4.15 | | | | | | | | | | | | | | | | | |
| LLB2. | . | . | . | E | I | . | A | . | N | . | Y | . | . | . | . | . | . |
| 10.8.4.8 | | | | | | | | | | | | | | | | | |
| LLB2. | . | . | . | E | I | . | A | . | N | . | Y | . | . | . | . | . | . |
| 10.8.4.12 | | | | | | | | | | | | | | | | | |
| LLB2. | . | . | . | E | I | . | A | . | N | . | Y | . | . | . | . | . | . |
| 10.8.14.3 | | | | | | | | | | | | | | | | | |
| LLB2. | . | . | . | E | I | . | A | . | N | . | Y | . | . | . | . | . | . |
| 10.8.14.4 | | | | | | | | | | | | | | | | | |
| LLB2. | . | . | . | E | I | . | A | . | N | . | Y | . | . | . | . | . | . |
| 10.8.14.12 | | | | | | | | | | | | | | | | | |
| LLB2. | . | . | . | E | I | . | A | . | N | . | Y | . | . | . | . | . | . |
| 10.8.5.1 | | | | | | | | | | | | | | | | | |
| LLB2. | . | . | . | E | . | . | A | . | . | . | Y | . | . | . | . | . | . |
| 10.8.5. | | | | | | | | | | | | | | | | | |
| 3star | | | | | | | | | | | | | | | | | |
| LLB2. | . | . | . | E | I | . | A | . | N | . | Y | . | . | . | . | . | . |
| 10.8.18.5 | | | | | | | | | | | | | | | | | |
| LLB2. | . | . | . | E | I | . | A | . | N | . | Y | . | . | . | . | . | . |
| 10.8.18.14 | | | | | | | | | | | | | | | | | |
| LLB2. | . | . | . | E | T | . | A | . | N | . | Y | . | . | . | . | . | . |
| 10.8.9.11 | | | | | | | | | | | | | | | | | |
| LLB2. | . | . | . | E | I | . | A | . | N | . | Y | . | . | . | . | . | . |
| 10.8.10.1 | | | | | | | | | | | | | | | | | |
| LLB2. | . | . | . | E | I | . | A | . | N | . | Y | . | . | . | . | . | . |
| 10.8.10.3 | | | | | | | | | | | | | | | | | |
| LLB2. | . | . | . | E | I | . | A | . | N | . | Y | . | . | . | . | . | . |
| 10.8.10.8 | | | | | | | | | | | | | | | | | |
| LLB2. | . | . | . | E | I | . | A | . | N | . | Y | . | . | . | . | . | . |
| 10.8.21 | | | | | | | | | | | | | | | | | |
| LLB2. | . | . | . | E | I | . | A | . | N | . | Y | . | . | . | . | . | . |
| 10.8.22 | | | | | | | | | | | | | | | | | |
| LLB2. | . | . | . | E | I | . | A | . | N | . | Y | . | . | . | . | . | . |
| 10.8.23 | | | | | | | | | | | | | | | | | |
| LLB2. | . | . | . | E | I | . | A | . | N | . | Y | . | . | . | . | . | . |
| 10.8.24 | | | | | | | | | | | | | | | | | |

| | 435 | 436 | 437 | 438 | 439 | 440 | 441 | 442 | 443 | 444 | 445 | 446 | 447 |
|---|---|---|---|---|---|---|---|---|---|---|---|---|---|
| WT Fc | H | Y | T | Q | K | S | L | S | L | S | P | G | K |
| Library | | Y | | F | | N | | A | | | | | |
| Scaffold | | | | | | | | | | | | | |
| Patch. 1 | | | | | | NNK | | | | | | | |
| Patch. 2 | | | | | | | | | | | | | |
| Patch. 3 | | | | | | | | | | | | | |
| Patch. 4 | | NNK | | NNK | | NNK | | NNK | | | | | |
| Patch. 5 | | | | | | | | | | | | | |
| Patch. 6 | | NNK | | | | | | NNK | | | | | |
| Patch. 7 | | | | | | | | | | | | | |
| Patch. 8 | | NNK | | | | | | NNK | | | | | |
| Patch. 9 | | | | NNK | | NNK | | | | | | | |
| Patch. 10 | | | | | | NNK | | NNK | | | | | |
| LLB2. | . | . | . | F | . | N | . | A | . | . | . | . | . |
| 10.8.2.1 | | | | | | | | | | | | | |
| LLB2. | . | . | . | F | . | N | . | A | . | . | . | . | . |
| 10.8.12.1 | | | | | | | | | | | | | |
| LLB2. | . | . | . | F | . | N | . | A | . | . | . | . | . |
| 10.8.2.3 | | | | | | | | | | | | | |
| LLB2. | . | . | . | F | . | N | . | A | . | . | . | . | . |
| 10.8.2.4 | | | | | | | | | | | | | |
| LLB2. | . | . | . | F | . | N | . | A | . | . | . | . | . |
| 10.8.12.5 | | | | | | | | | | | | | |
| LLB2. | . | . | . | F | . | N | . | A | . | . | . | . | . |
| 10.8.2.15 | | | | | | | | | | | | | |
| LLB2. | . | R | . | F | . | N | . | K | . | . | . | . | . |
| 10.8.4.15 | | | | | | | | | | | | | |
| LLB2. | . | R | . | W | . | N | . | K | . | . | . | . | . |
| 10.8.4.8 | | | | | | | | | | | | | |
| LLB2. | . | W | . | F | . | N | . | R | . | . | . | . | . |
| 10.8.4.12 | | | | | | | | | | | | | |
| LLB2. | . | R | . | F | . | N | . | R | . | . | . | . | . |
| 10.8.14.3 | | | | | | | | | | | | | |
| LLB2. | . | R | . | F | . | N | . | A | . | . | . | . | . |
| 10.8.14.4 | | | | | | | | | | | | | |
| LLB2. | . | R | . | W | . | N | . | A | . | . | . | . | . |
| 10.8.14.12 | | | | | | | | | | | | | |
| LLB2. | . | . | . | F | . | N | . | A | . | . | . | . | . |
| 10.8.5.1 | | | | | | | | | | | | | |
| LLB2. | . | . | . | F | . | N | . | A | . | . | . | . | . |
| 10.8.5. | | | | | | | | | | | | | |
| 3star | | | | | | | | | | | | | |
| LLB2. | . | R | . | F | . | N | . | K | . | . | . | . | . |
| 10.8.18.5 | | | | | | | | | | | | | |
| LLB2. | . | W | . | F | . | N | . | H | . | . | . | . | . |
| 10.8.18.14 | | | | | | | | | | | | | |
| LLB2. | . | . | . | F | . | N | . | A | . | . | . | . | . |
| 10.8.9.11 | | | | | | | | | | | | | |
| LLB2. | . | . | . | F | . | N | . | K | . | . | . | . | . |
| 10.8.10.1 | | | | | | | | | | | | | |
| LLB2. | . | . | . | F | . | N | . | R | . | . | . | . | . |
| 10.8.10.3 | | | | | | | | | | | | | |
| LLB2. | . | . | . | F | . | N | . | H | . | . | . | . | . |
| 10.8.10.8 | | | | | | | | | | | | | |
| LLB2. | . | R | . | F | . | N | . | K | . | . | . | . | . |
| 10.8.21 | | | | | | | | | | | | | |
| LLB2. | . | R | . | W | . | N | . | A | . | . | . | . | . |
| 10.8.22 | | | | | | | | | | | | | |
| LLB2. | . | R | . | W | . | N | . | K | . | . | . | . | . |
| 10.8.23 | | | | | | | | | | | | | |
| LLB2. | . | W | . | F | . | N | . | R | . | . | . | . | . |
| 10.8.24 | | | | | | | | | | | | | |

TABLE A8
LLB2 library for affinity dematuration engineering round using rational design

| | 376 | 377 | 378 | 379 | 380 | 381 | 382 | 383 | 384 | 385 | 386 | 387 | 388 | 389 | 390 | 390 | 392 |
|---|---|---|---|---|---|---|---|---|---|---|---|---|---|---|---|---|---|
| WT Fc | D | I | A | V | E | W | E | S | N | G | Q | P | E | N | N | Y | K |
| LLB2 | | | | | L | | N | | X | X | X | L | | | | | |
| Scaffold | | | | | | | | | | | | | | | | | |
| | | | | | | | | | HR | FY | EV | | | | | | |
| LLB2. | . | . | . | . | L | . | N | . | H | Y | E | L | . | . | . | . | . |
| 10.8.12. | | | | | | | | | | | | | | | | | |
| 5.N | | | | | | | | | | | | | | | | | |
| LLB2. | . | . | . | . | L | . | N | . | R | F | V | L | . | . | . | . | . |
| 10.8.4. | | | | | | | | | | | | | | | | | |
| 12.N | | | | | | | | | | | | | | | | | |
| LLB2. | . | . | . | . | L | . | N | . | R | F | V | L | . | . | . | . | . |
| 10.8.14. | | | | | | | | | | | | | | | | | |
| 3.N | | | | | | | | | | | | | | | | | |
| LLB2. | . | . | . | . | L | . | N | . | R | F | V | L | . | . | . | . | . |
| 10.8.14. | | | | | | | | | | | | | | | | | |
| 4.N | | | | | | | | | | | | | | | | | |
| LLB2. | . | . | . | . | L | . | N | . | R | F | V | L | . | . | . | . | . |
| 10.8.9. | | | | | | | | | | | | | | | | | |
| 11.N | | | | | | | | | | | | | | | | | |
| LLB2. | . | . | . | . | L | . | N | . | R | F | V | L | . | . | . | . | . |
| 10.8.10. | | | | | | | | | | | | | | | | | |
| 1.N | | | | | | | | | | | | | | | | | |
| LLB2. | . | . | . | . | L | . | N | . | R | F | V | L | . | . | . | . | . |
| 10.8.10. | | | | | | | | | | | | | | | | | |
| 3.N | | | | | | | | | | | | | | | | | |
| LLB2. | . | . | . | . | L | . | N | . | R | F | V | L | . | . | . | . | . |
| 10.8.10. | | | | | | | | | | | | | | | | | |
| 8.N | | | | | | | | | | | | | | | | | |
| LLB2- | . | . | . | . | L | . | N | . | R | F | V | L | . | . | . | . | . |
| 10-8-d1 MV | | | | | | | | | | | | | | | | | |
| LLB2- | . | . | . | . | L | . | N | . | R | F | V | L | . | . | . | . | . |
| 10-8-d2 MV | | | | | | | | | | | | | | | | | |
| LLB2- | . | . | . | . | L | . | N | . | R | F | V | L | . | . | . | . | . |
| 10-8-d3 MV | | | | | | | | | | | | | | | | | |
| LLB2- | . | . | . | . | L | . | N | . | R | F | V | L | . | . | . | . | . |
| 10-8-d4 MV | | | | | | | | | | | | | | | | | |
| LLB2- | . | . | . | . | L | . | N | . | R | F | V | L | . | . | . | . | . |
| 10-8-d5 MV | | | | | | | | | | | | | | | | | |
| LLB2- | . | . | . | . | L | . | N | . | R | F | V | L | . | . | . | . | . |
| 10-8-d6 MV | | | | | | | | | | | | | | | | | |
| LLB2- | . | . | . | . | L | . | N | . | R | F | V | L | . | . | . | . | . |
| 10-8-d1 BV | | | | | | | | | | | | | | | | | |
| LLB2- | . | . | . | . | L | . | N | . | R | F | V | L | . | . | . | . | . |
| 10-8-d2 BV | | | | | | | | | | | | | | | | | |
| LLB2- | . | . | . | . | L | . | N | . | R | F | V | L | . | . | . | . | . |
| 10-8-d3 BV | | | | | | | | | | | | | | | | | |
| LLB2- | . | . | . | . | L | . | N | . | R | F | V | L | . | . | . | . | . |
| 10-8-d4 BV | | | | | | | | | | | | | | | | | |
| LLB2- | . | . | . | . | L | . | N | . | R | F | V | L | . | . | . | . | . |
| 10-8-d5 BV | | | | | | | | | | | | | | | | | |
| LLB2- | . | . | . | . | L | . | N | . | R | F | V | L | . | . | . | . | . |
| 10-8-d6 BV | | | | | | | | | | | | | | | | | |
| LLB2- | . | . | . | . | L | . | N | . | H | Y | E | L | . | . | . | . | . |
| 10-8-d7 BV | | | | | | | | | | | | | | | | | |
| LLB2- | . | . | . | . | L | . | N | . | H | Y | E | L | . | . | . | . | . |
| 10-8-d8 BV | | | | | | | | | | | | | | | | | |
| LLB2- | . | . | . | . | L | . | N | . | H | Y | E | L | . | . | . | . | . |
| 10-8-d9 BV | | | | | | | | | | | | | | | | | |
| LLB2- | . | . | . | . | L | . | N | . | H | Y | E | L | . | . | . | . | . |
| 10-8-d10 BV | | | | | | | | | | | | | | | | | |
| LLB2- | . | . | . | . | L | . | N | . | H | Y | E | L | . | . | . | . | . |
| 10-8-d11 BV | | | | | | | | | | | | | | | | | |
| LLB2- | . | . | . | . | L | . | N | . | H | Y | E | L | . | . | . | . | . |
| 10-8-d12 BV | | | | | | | | | | | | | | | | | |
| LLB2- | . | . | . | . | L | . | N | . | Q | Y | E | L | . | . | . | . | . |
| 10-8-d13 BV | | | | | | | | | | | | | | | | | |
| LLB2- | . | . | . | . | L | . | N | . | Q | Y | E | L | . | . | . | . | . |
| 10-8-d14 BV | | | | | | | | | | | | | | | | | |
| LLB2- | . | . | . | . | L | . | N | . | Q | Y | E | L | . | . | . | . | . |
| 10-8-d15 BV | | | | | | | | | | | | | | | | | |
| LLB2- | . | . | . | . | L | . | N | . | Q | Y | E | L | . | . | . | . | . |
| 10-8-d16 BV | | | | | | | | | | | | | | | | | |
| LLB2- | . | . | . | . | L | . | N | . | Q | Y | E | L | . | . | . | . | . |
| 10-8-d17 BV | | | | | | | | | | | | | | | | | |
| LLB2- | . | . | . | . | L | . | N | . | Q | Y | E | L | . | . | . | . | . |
| 10-8-d18 BV | | | | | | | | | | | | | | | | | |
| | 418 | 419 | 420 | 421 | 422 | 423 | 424 | 425 | 426 | 427 | 428 | 429 | 430 | 431 | 432 | 433 | 434 |
| WT Fc | Q | Q | G | N | V | F | S | C | S | V | M | H | E | A | L | H | N |
| LLB2 | | | | X | X | | A | | N | | Y | | | | | | |
| Scaffold | | | | | | | | | | | | | | | | | |
| | | | | NE | IVT | | | | | | | | | | | | |
| LLB2. | . | . | . | . | I | . | A | . | N | . | Y | . | . | . | . | . | . |
| 10.8.12. | | | | | | | | | | | | | | | | | |
| 5.N | | | | | | | | | | | | | | | | | |
| LLB2. | . | . | . | . | I | . | A | . | N | . | Y | . | . | . | . | . | . |
| 10.8.4. | | | | | | | | | | | | | | | | | |
| 12.N | | | | | | | | | | | | | | | | | |
| LLB2. | . | . | . | . | I | . | A | . | N | . | Y | . | . | . | . | . | . |
| 10.8.14. | | | | | | | | | | | | | | | | | |
| 3.N | | | | | | | | | | | | | | | | | |
| LLB2. | . | . | . | . | I | . | A | . | N | . | Y | . | . | . | . | . | . |
| 10.8.14. | | | | | | | | | | | | | | | | | |
| 4.N | | | | | | | | | | | | | | | | | |
| LLB2. | . | . | . | . | I | . | A | . | N | . | Y | . | . | . | . | . | . |
| 10.8.9. | | | | | | | | | | | | | | | | | |
| 11.N | | | | | | | | | | | | | | | | | |
| LLB2. | . | . | . | . | I | . | A | . | N | . | Y | . | . | . | . | . | . |
| 10.8.10. | | | | | | | | | | | | | | | | | |
| 1.N | | | | | | | | | | | | | | | | | |
| LLB2. | . | . | . | . | I | . | A | . | N | . | Y | . | . | . | . | . | . |
| 10.8.10. | | | | | | | | | | | | | | | | | |
| 3.N | | | | | | | | | | | | | | | | | |
| LLB2. | . | . | . | . | I | . | A | . | N | . | Y | . | . | . | . | . | . |
| 10.8.10. | | | | | | | | | | | | | | | | | |
| 8.N | | | | | | | | | | | | | | | | | |
| LLB2- | . | . | . | E | I | . | A | . | N | . | Y | . | . | . | . | . | . |
| 10-8-d1 MV | | | | | | | | | | | | | | | | | |
| LLB2- | . | . | . | E | . | . | A | . | N | . | Y | . | . | . | . | . | . |
| 10-8-d2 MV | | | | | | | | | | | | | | | | | |
| LLB2- | . | . | . | E | . | . | A | . | N | . | Y | . | . | . | . | . | . |
| 10-8-d3 MV | | | | | | | | | | | | | | | | | |
| LLB2- | . | . | . | . | I | . | A | . | N | . | Y | . | . | . | . | . | . |
| 10-8-d4 MV | | | | | | | | | | | | | | | | | |
| LLB2- | . | . | . | . | . | . | A | . | N | . | Y | . | . | . | . | . | . |
| 10-8-d5 MV | | | | | | | | | | | | | | | | | |
| LLB2- | . | . | . | . | . | . | A | . | N | . | Y | . | . | . | . | . | . |
| 10-8-d6 MV | | | | | | | | | | | | | | | | | |
| LLB2- | . | . | . | E | I | . | A | . | N | . | Y | . | . | . | . | . | . |
| 10-8-d1 BV | | | | | | | | | | | | | | | | | |
| LLB2- | . | . | . | E | . | . | A | . | N | . | Y | . | . | . | . | . | . |
| 10-8-d2 BV | | | | | | | | | | | | | | | | | |
| LLB2- | . | . | . | E | . | . | A | . | N | . | Y | . | . | . | . | . | . |
| 10-8-d3 BV | | | | | | | | | | | | | | | | | |
| LLB2- | . | . | . | . | I | . | A | . | N | . | Y | . | . | . | . | . | . |
| 10-8-d4 BV | | | | | | | | | | | | | | | | | |
| LLB2- | . | . | . | . | | . | A | . | N | . | Y | . | . | . | . | . | . |
| 10-8-d5 BV | | | | | | | | | | | | | | | | | |
| LLB2- | . | . | . | . | . | . | A | . | N | . | Y | . | . | . | . | . | . |
| 10-8-d6 BV | | | | | | | | | | | | | | | | | |
| LLB2- | . | . | . | E | I | . | A | . | N | . | Y | . | . | . | . | . | . |
| 10-8-d7 BV | | | | | | | | | | | | | | | | | |
| LLB2- | . | . | . | E | . | . | A | . | N | . | Y | . | . | . | . | . | . |
| 10-8-d8 BV | | | | | | | | | | | | | | | | | |
| LLB2- | . | . | . | E | . | . | A | . | N | . | Y | . | . | . | . | . | . |
| 10-8-d9 BV | | | | | | | | | | | | | | | | | |
| LLB2- | . | . | . | . | I | . | A | . | N | . | Y | . | . | . | . | . | . |
| 10-8-d10 BV | | | | | | | | | | | | | | | | | |
| LLB2- | . | . | . | . | . | . | A | . | N | . | Y | . | . | . | . | . | . |
| 10-8-d11 BV | | | | | | | | | | | | | | | | | |
| LLB2- | . | . | . | . | . | . | A | . | N | . | Y | . | . | . | . | . | . |
| 10-8-d12 BV | | | | | | | | | | | | | | | | | |
| LLB2- | . | . | . | E | I | . | A | . | N | . | Y | . | . | . | . | . | . |
| 10-8-d13 BV | | | | | | | | | | | | | | | | | |
| LLB2- | . | . | . | E | . | . | A | . | N | . | Y | . | . | . | . | . | . |
| 10-8-d14 BV | | | | | | | | | | | | | | | | | |
| LLB2- | . | . | . | E | . | . | A | . | N | . | Y | . | . | . | . | . | . |
| 10-8-d15 BV | | | | | | | | | | | | | | | | | |
| LLB2- | . | . | . | . | I | . | A | . | N | . | Y | . | . | . | . | . | . |
| 10-8-d16 BV | | | | | | | | | | | | | | | | | |
| LLB2- | . | . | . | . | . | . | A | . | N | . | Y | . | . | . | . | . | . |
| 10-8-d17 BV | | | | | | | | | | | | | | | | | |
| LLB2- | . | . | . | . | . | . | A | . | N | . | Y | . | . | . | . | . | . |
| 10-8-d18 BV | | | | | | | | | | | | | | | | | |

| | 435 | 436 | 437 | 438 | 439 | 440 | 441 | 442 | 443 | 444 | 445 | 446 | 447 |
|---|---|---|---|---|---|---|---|---|---|---|---|---|---|
| WT Fc | H | Y | T | Q | K | S | L | S | L | S | P | G | K |
| LLB2 | | X | | F | | N | | X | | | | | |
| Scaffold | | | | | | | | | | | | | |
| | | YWR | | | | | | SARKH | | | | | |
| LLB2. | . | . | . | F | . | N | . | A | . | . | . | . | . |
| 10.8.12. | | | | | | | | | | | | | |
| 5.N | | | | | | | | | | | | | |
| LLB2. | . | W | . | F | . | N | . | R | . | . | . | . | . |
| 10.8.4. | | | | | | | | | | | | | |
| 12.N | | | | | | | | | | | | | |
| LLB2. | . | R | . | F | . | N | . | R | . | . | . | . | . |
| 10.8.14. | | | | | | | | | | | | | |
| 3.N | | | | | | | | | | | | | |
| LLB2. | . | R | . | F | . | N | . | A | . | . | . | . | . |
| 10.8.14. | | | | | | | | | | | | | |
| 4.N | | | | | | | | | | | | | |
| LLB2. | . | . | . | F | . | N | . | A | . | . | . | . | . |
| 10.8.9. | | | | | | | | | | | | | |
| 11.N | | | | | | | | | | | | | |
| LLB2. | . | . | . | F | . | N | . | K | . | . | . | . | . |
| 10.8.10. | | | | | | | | | | | | | |
| 1.N | | | | | | | | | | | | | |
| LLB2. | . | . | . | F | . | N | . | R | . | . | . | . | . |
| 10.8.10. | | | | | | | | | | | | | |
| 3.N | | | | | | | | | | | | | |
| LLB2. | . | . | . | F | . | N | . | H | . | . | . | . | . |
| 10.8.10. | | | | | | | | | | | | | |
| 8.N | | | | | | | | | | | | | |
| LLB2- | . | . | . | F | . | N | . | . | . | . | . | . | . |
| 10-8-d1 MV | | | | | | | | | | | | | |
| LLB2- | . | . | . | F | . | N | . | . | . | . | . | . | . |
| 10-8-d2 MV | | | | | | | | | | | | | |
| LLB2- | . | . | . | F | . | N | . | A | . | . | . | . | . |
| 10-8-d3 MV | | | | | | | | | | | | | |
| LLB2- | . | . | . | F | . | N | . | . | . | . | . | . | . |
| 10-8-d4 MV | | | | | | | | | | | | | |
| LLB2- | . | . | . | F | . | N | . | . | . | . | . | . | . |
| 10-8-d5 MV | | | | | | | | | | | | | |
| LLB2- | . | . | . | F | . | N | . | A | . | . | . | . | . |
| 10-8-d6 MV | | | | | | | | | | | | | |
| LLB2- | . | . | . | F | . | N | . | . | . | . | . | . | . |
| 10-8-d1 BV | | | | | | | | | | | | | |
| LLB2- | . | . | . | F | . | N | . | . | . | . | . | . | . |
| 10-8-d2 BV | | | | | | | | | | | | | |
| LLB2- | . | . | . | F | . | N | . | A | . | . | . | . | . |
| 10-8-d3 BV | | | | | | | | | | | | | |
| LLB2- | . | . | . | F | . | N | . | . | . | . | . | . | . |
| 10-8-d4 BV | | | | | | | | | | | | | |
| LLB2- | . | . | . | F | . | N | . | . | . | . | . | . | . |
| 10-8-d5 BV | | | | | | | | | | | | | |
| LLB2- | . | . | . | F | . | N | . | A | . | . | . | . | . |
| 10-8-d6 BV | | | | | | | | | | | | | |
| LLB2- | . | . | . | F | . | N | . | . | . | . | . | . | . |
| 10-8-d7 BV | | | | | | | | | | | | | |
| LLB2- | . | . | . | F | . | N | . | . | . | . | . | . | . |
| 10-8-d8 BV | | | | | | | | | | | | | |
| LLB2- | . | . | . | F | . | N | . | A | . | . | . | . | . |
| 10-8-d9 BV | | | | | | | | | | | | | |
| LLB2- | . | . | . | F | . | N | . | . | . | . | . | . | . |
| 10-8-d10 BV | | | | | | | | | | | | | |
| LLB2- | . | . | . | F | . | N | . | . | . | . | . | . | . |
| 10-8-d11 BV | | | | | | | | | | | | | |
| LLB2- | . | . | . | F | . | N | . | A | . | . | . | . | . |
| 10-8-d12 BV | | | | | | | | | | | | | |
| LLB2- | . | . | . | F | . | N | . | . | . | . | . | . | . |
| 10-8-d13 BV | | | | | | | | | | | | | |
| LLB2- | . | . | . | F | . | N | . | . | . | . | . | . | . |
| 10-8-d14 BV | | | | | | | | | | | | | |
| LLB2- | . | . | . | F | . | N | . | A | . | . | . | . | . |
| 10-8-d15 BV | | | | | | | | | | | | | |
| LLB2- | . | . | . | F | . | N | . | . | . | . | . | . | . |
| 10-8-d16 BV | | | | | | | | | | | | | |
| LLB2- | . | . | . | F | . | N | . | . | . | . | . | . | . |
| 10-8-d17 BV | | | | | | | | | | | | | |
| LLB2- | . | . | . | F | . | N | . | A | . | . | . | . | . |
| 10-8-d18 BV | | | | | | | | | | | | | |

TABLE A12
LLB2 library for additional affinity dematuration engineering round

| | 378 | 379 | 380 | 381 | 382 | 383 | 384 | 385 | 386 | 387 | 388 | 389 | 390 | 390 | 392 | 418 |
|---|---|---|---|---|---|---|---|---|---|---|---|---|---|---|---|---|
| WT Fc | A | V | E | W | E | S | N | G | Q | P | E | N | N | Y | K | Q |
| LLB2- | | | L | | N | | K | F | E | L | | | | | | |
| 10-8.d19 | | | | | | | | | | | | | | | | |
| LLB2- | | | L | | S | | H | Y | E | L | | | | | | |
| 10-8.d20 | | | | | | | | | | | | | | | | |
| LLB2- | | | L | | S | | R | F | V | L | | | | | | |
| 10-8.d21 | | | | | | | | | | | | | | | | |
| LLB2- | | | L | | S | | K | F | E | L | | | | | | |
| 10-8.d22 | | | | | | | | | | | | | | | | |
| LLB2- | | | L | | S | | Q | Y | E | L | | | | | | |
| 10-8.d23 | | | | | | | | | | | | | | | | |
| LLB2- | | | L | | N | | R | Y | V | L | | | | | | |
| 10-8.d24 | | | | | | | | | | | | | | | | |
| LLB2- | | | L | | N | | R | F | . | L | | | | | | |
| 10-8.d25 | | | | | | | | | | | | | | | | |
| LLB2- | | | L | | N | | R | F | V | . | | | | | | |
| 10-8.d26 | | | | | | | | | | | | | | | | |
| LLB2- | | | L | | N | | R | F | . | . | | | | | | |
| 10-8.d27 | | | | | | | | | | | | | | | | |
| LLB2- | | | L | | S | | R | Y | V | L | | | | | | |
| 10-8.d28 | | | | | | | | | | | | | | | | |
| LLB2- | | | L | . | S | . | R | F | . | L | | | | | | |
| 10-8.d29 | | | | | | | | | | | | | | | | |
| LLB2- | | | L | . | N | . | Q | F | E | L | . | . | . | . | . | |
| 10-8.d30 | | | | | | | | | | | | | | | | |
| LLB2- | | | L | . | N | . | Q | Y | . | L | . | . | . | . | . | |
| 10-8.d31 | | | | | | | | | | | | | | | | |
| LLB2- | | | L | . | S | . | Q | F | E | L | . | . | . | . | . | |
| 10-8.d32 | | | | | | | | | | | | | | | | |
| | 419 | 420 | 421 | 422 | 423 | 424 | 425 | 426 | 427 | 428 | 429 | 430 | 431 | 432 | 433 | 434 |
| WT Fc | Q | G | N | V | F | S | C | S | V | M | H | E | A | L | H | N |
| LLB2- | | | | | | A | | N | | Y | | | | | | |
| 10-8.d19 | | | | | | | | | | | | | | | | |
| LLB2- | | | | | | A | | N | | Y | | | | | | |
| 10-8.d20 | | | | | | | | | | | | | | | | |
| LLB2- | | | | | | A | | N | | Y | | | | | | |
| 10-8.d21 | | | | | | | | | | | | | | | | |
| LLB2- | | | | | | A | | N | | Y | | | | | | |
| 10-8.d22 | | | | | | | | | | | | | | | | |
| LLB2- | | | | | | A | | N | | Y | | | | | | |
| 10-8.d23 | | | | | | | | | | | | | | | | |
| LLB2- | | | | | | A | | N | | Y | | | | | | |
| 10-8.d24 | | | | | | | | | | | | | | | | |
| LLB2- | | | | | | A | | N | | Y | | | | | | |
| 10-8.d25 | | | | | | | | | | | | | | | | |
| LLB2- | | | | | | A | | N | | Y | | | | | | |
| 10-8.d26 | | | | | | | | | | | | | | | | |
| LLB2- | | | | | | A | | N | | Y | | | | | | |
| 10-8.d27 | | | | | | | | | | | | | | | | |
| LLB2- | | | | | | A | | N | | Y | | | | | | |
| 10-8.d28 | | | | | | | | | | | | | | | | |
| LLB2- | | | | | | A | | N | | Y | | | | | | |
| 10-8.d29 | | | | | | | | | | | | | | | | |
| LLB2- | | | | | . | A | | N | | Y | | | | | | |
| 10-8.d30 | | | | | | | | | | | | | | | | |
| LLB2- | | | | | . | A | | N | | Y | | | | | | |
| 10-8.d31 | | | | | | | | | | | | | | | | |
| LLB2- | | | | | . | A | | N | | Y | | | | | | |
| 10-8.d32 | | | | | | | | | | | | | | | | |

| | 435 | 436 | 437 | 438 | 439 | 440 | 441 | 442 | 443 | 444 | 445 | 446 | 447 |
|---|---|---|---|---|---|---|---|---|---|---|---|---|---|
| WT Fc | H | Y | T | Q | K | S | L | S | L | S | P | G | K |
| LLB2- | | | | F | | N | | A | | | | | |
| 10-8.d19 | | | | | | | | | | | | | |
| LLB2- | | | | F | | N | | A | | | | | |
| 10-8.d20 | | | | | | | | | | | | | |
| LLB2- | | | | F | | N | | A | | | | | |
| 10-8.d21 | | | | | | | | | | | | | |
| LLB2- | | | | F | | N | | A | | | | | |
| 10-8.d22 | | | | | | | | | | | | | |
| LLB2- | | | | F | | N | | A | | | | | |
| 10-8.d23 | | | | | | | | | | | | | |
| LLB2- | | | | F | | N | | A | | | | | |
| 10-8.d24 | | | | | | | | | | | | | |
| LLB2- | | | | F | | N | | A | | | | | |
| 10-8.d25 | | | | | | | | | | | | | |
| LLB2- | | | | F | | N | | A | | | | | |
| 10-8.d26 | | | | | | | | | | | | | |
| LLB2- | | | | F | | N | | A | | | | | |
| 10-8.d27 | | | | | | | | | | | | | |
| LLB2- | | | | F | | N | | A | | | | | |
| 10-8.d28 | | | | | | | | | | | | | |
| LLB2- | | | | F | | N | | A | | | | | |
| 10-8.d29 | | | | | | | | | | | | | |
| LLB2- | | | | F | | N | | A | | | | | |
| 10-8.d30 | | | | | | | | | | | | | |
| LLB2- | | | | F | | N | | A | | | | | |
| 10-8.d31 | | | | | | | | | | | | | |
| LLB2- | | | | F | | N | | A | | | | | |
| 10-8.d32 | | | | | | | | | | | | | |

### Example 3. Generation of LLB1 Family CD98HC Binders

### LLB1 Family Affinity Maturation (LLB1-AM1 and LLB1-AM2)

The residues found in the clones of the LLB1 family (i.e., LLB1, LLB6 and LLB7) were used to create two phage display libraries (LLB1-AM1 and LLB1-AM2) to increase the affinity to CD98hc (Table 10). LLB1-AM1 did not expand the number of positions in the library. The LLB1-AM2 expanded to residues 428 and 434. These libraries were created using Kunkel mutagenesis and screened to human CD98hc using phage display. There were 16 clones that were selected for recombinant expression with anti-BACE1 Fabs with full effector function and in a bivalent format (i.e., no knob and hole mutations). The sequences of the clones are shown in Table A9. These clones were shown to bind to human CD98hc but not cyno CD98hc. The lead clone was reformatted in a monovalent format, called LLB1-3 monovalent, with anti-BACE1 Fabs having full effector function and with the CD98hc binding site on the knob side only, and its affinity to human CD98hc was measured by surface plasmon resonance (SPR) using a Biacore™ machine. The valency of the CD98hc-binding molecule did not have a great impact on the affinity to CD98hc, i.e., less than a 2-fold difference. Cell binding of the clones was also tested in HeLa cells for huCD98hc binding, CHO:cyCD98hc cells for cyno binding, and CHO cells as a negative control.

TABLE 10
LLB1-AMI and LLB2-AM2 Affinity Maturation Library

| | | 380 | 382 | 384 | 385 | 386 | 387 | |
|---|---|---|---|---|---|---|---|---|
| WT Fc | | E | E | N | G | Q | P | |
| LLB1-AMI | AA | X2 | X1 | X2 | X1 | X2 | X1 | |
| | Codons | NHK | NNK | NHK | NNK | NHK | NNK | |
| LLB1-AM2 | AA | D | RNSK | Y | YFLH | FTSI | RT | |
| | Codons | GAT | ARN | TAT | YWY | WYY | ASR | |
| | | 422 | 424 | 426 | 428 | 434 | 438 | 440 |
| WT Fc | | V | S | S | M | N | Q | S |
| LLB1-AMI | AA | KI | VLWG | DSYA | | | FI | TK |
| | Codons | AWA | KKG | KMY | | | WTY | AMR |
| LLB1-AM2 | AA | X1 | X1 | X1 | ML | NS | X1 | X1 |
| | Codons | NNK | NNK | NNK | MTG | ARY | NNK | NNK |

X1 = any amino acid
X2 = any amino acid except Arginine, Tryptophan, Glycine and Cysteine

TABLE A9
LLB1 affinity maturation library AM1 and AM2

| | 369 | 370 | 371 | 372 | 373 | 374 | 375 | 376 | 377 | 378 | 379 | 380 | 381 | 382 | 383 | 384 | 385 | 386 |
|---|---|---|---|---|---|---|---|---|---|---|---|---|---|---|---|---|---|---|
| WT Fc | V | K | G | F | Y | P | S | D | I | A | V | E | W | E | S | N | G | Q |
| LLB1-1 | . | . | . | . | . | . | . | . | . | . | . | D | . | R | . | L | F | T |
| LLB1-2 | . | . | . | . | . | . | . | . | . | . | . | D | . | R | . | Y | F | P |
| LLB1-3 | . | . | . | . | . | . | . | . | . | . | . | D | . | R | . | Y | K | P |
| LLB1-4 | . | . | . | . | . | . | . | . | . | . | . | M | . | Y | . | A | D | E |
| LLB1-5 | . | . | . | . | . | . | . | . | . | . | . | D | . | R | . | Y | . | K |
| LLB1-6 | . | . | . | . | . | . | . | . | . | . | . | N | . | F | . | . | M | K |
| LLB1-7 | . | . | . | . | . | . | . | . | . | . | . | N | . | S | . | S | I | A |
| LLB1-8 | . | . | . | . | . | . | . | . | . | . | . | P | . | W | . | L | N | V |
| LLB1-9 | . | . | . | . | . | . | . | . | . | . | . | F | . | Y | . | F | Y | V |
| LLB1- | . | . | . | . | . | . | . | . | . | . | . | H | . | W | . | L | F | D |
| 10 | | | | | | | | | | | | | | | | | | |
| LLB1- | . | . | . | . | . | . | . | . | . | . | . | D | . | R | . | Y | F | T |
| 11 | | | | | | | | | | | | | | | | | | |
| LLB1- | . | . | . | . | . | . | . | . | . | . | . | D | . | R | . | Y | Y | T |
| 12 | | | | | | | | | | | | | | | | | | |
| LLB1- | . | . | . | . | . | . | . | . | . | . | . | . | . | N | . | Y | L | F |
| 13 | | | | | | | | | | | | | | | | | | |
| LLB1- | . | . | . | . | . | . | . | . | . | . | . | D | . | K | . | Y | L | F |
| 14 | | | | | | | | | | | | | | | | | | |
| LLB1- | . | . | . | . | . | . | . | . | . | . | . | D | . | N | . | Y | F | F |
| 15 | | | | | | | | | | | | | | | | | | |
| LLB1- | . | . | . | . | . | . | . | . | . | . | . | D | . | S | . | Y | H | F |
| 16 | | | | | | | | | | | | | | | | | | |
| LLB1-2 | . | . | . | . | . | . | . | . | . | . | . | D | . | R | . | Y | K | P |
| Mono- | | | | | | | | | | | | | | | | | | |
| valent | | | | | | | | | | | | | | | | | | |
| | 387 | 388 | 389 | 390 | 391 | 392 | 418 | 419 | 420 | 421 | 422 | 423 | 424 | 425 | 426 | 427 | 428 | 429 |
| WT Fc | P | E | N | N | Y | K | Q | Q | G | N | V | F | S | C | S | V | M | H |
| LLB1-1 | N | . | . | . | . | . | . | . | . | . | I | . | V | . | D | . | . | . |
| LLB1-2 | L | . | . | . | . | . | . | . | . | . | I | . | V | . | D | . | . | . |
| LLB1-3 | Y | . | . | . | . | . | . | . | . | . | I | . | V | . | D | . | . | . |
| LLB1-4 | R | . | . | . | . | . | . | . | . | . | K | . | W | . | . | . | . | . |
| LLB1-5 | G | . | . | . | . | . | . | . | . | . | I | . | V | . | D | . | . | . |
| LLB1-6 | Y | . | . | . | . | . | . | . | . | . | K | . | G | . | . | . | . | . |
| LLB1-7 | L | . | . | . | . | . | . | . | . | . | K | . | W | . | D | . | . | . |
| LLB1-8 | N | . | . | . | . | . | . | . | . | . | I | . | V | . | . | . | . | . |
| LLB1-9 | S | . | . | . | . | . | . | . | . | . | I | . | L | . | D | . | . | . |
| LLB1- | D | . | . | . | . | . | . | . | . | . | I | . | V | . | A | . | . | . |
| 10 | | | | | | | | | | | | | | | | | | |
| LLB1- | T | . | . | . | . | . | . | . | . | . | R | . | V | . | D | . | L | . |
| 11 | | | | | | | | | | | | | | | | | | |
| LLB1- | R | . | . | . | . | . | . | . | . | . | T | . | V | . | D | . | L | . |
| 12 | | | | | | | | | | | | | | | | | | |
| LLB1- | R | . | . | . | . | . | . | . | . | . | I | . | I | . | Q | . | . | . |
| 13 | | | | | | | | | | | | | | | | | | |
| LLB1- | T | . | . | . | . | . | . | . | . | . | F | . | P | . | W | . | L | . |
| 14 | | | | | | | | | | | | | | | | | | |
| LLB1- | R | . | . | . | . | . | . | . | . | . | H | . | W | . | L | . | . | . |
| 15 | | | | | | | | | | | | | | | | | | |
| LLB1- | R | . | . | . | . | . | . | . | . | . | . | . | Y | . | P | . | L | . |
| 16 | | | | | | | | | | | | | | | | | | |
| LLB1-2 | Y | . | . | . | . | . | . | . | . | . | I | . | V | . | D | . | . | . |
| Mono- | | | | | | | | | | | | | | | | | | |
| valent | | | | | | | | | | | | | | | | | | |
| | 430 | 431 | 432 | 433 | 434 | 435 | 436 | 437 | 438 | 439 | 440 | 441 | 442 | 443 | 444 | 445 | 446 | 447 |
| WT Fc | E | A | L | H | N | H | Y | T | Q | K | S | L | S | L | S | P | G | K |
| LLB1-1 | . | . | . | . | . | . | . | . | I | . | K | . | . | . | . | . | . | . |
| LLB1-2 | . | . | . | . | . | . | . | . | I | . | K | . | . | . | . | . | . | . |
| LLB1-3 | . | . | . | . | . | . | . | . | I | . | K | . | . | . | . | . | . | . |
| LLB1-4 | . | . | . | . | . | . | . | . | F | . | K | . | . | . | . | . | . | . |
| LLB1-5 | . | . | . | . | . | . | . | . | I | . | K | . | . | . | . | . | . | . |
| LLB1-6 | . | . | . | . | . | . | . | . | I | . | T | . | . | . | . | . | . | . |
| LLB1-7 | . | . | . | . | . | . | . | . | F | . | T | . | . | . | . | . | . | . |
| LLB1-8 | . | . | . | . | . | . | . | . | F | . | T | . | . | . | . | . | . | . |
| LLB1-9 | . | . | . | . | . | . | . | . | F | . | T | . | . | . | . | . | . | . |
| LLB1- | . | . | . | . | . | . | . | . | F | . | T | . | . | . | . | . | . | . |
| 10 | | | | | | | | | | | | | | | | | | |
| LLB1- | . | . | . | . | . | . | . | . | I | . | I | . | . | . | . | . | . | . |
| 11 | | | | | | | | | | | | | | | | | | |
| LLB1- | . | . | . | . | . | . | . | . | I | . | K | . | . | . | . | . | . | . |
| 12 | | | | | | | | | | | | | | | | | | |
| LLB1- | . | . | . | . | . | . | . | . | F | . | K | . | . | . | . | . | . | . |
| 13 | | | | | | | | | | | | | | | | | | |
| LLB1- | . | . | . | . | S | . | . | . | N | . | F | . | . | . | . | . | . | . |
| 14 | | | | | | | | | | | | | | | | | | |
| LLB1- | . | . | . | . | S | . | . | . | P | . | F | . | . | . | . | . | . | . |
| 15 | | | | | | | | | | | | | | | | | | |
| LLB1- | . | . | . | . | . | . | . | . | S | . | F | . | . | . | . | . | . | . |
| 16 | | | | | | | | | | | | | | | | | | |
| LLB1-2 | . | . | . | . | . | . | . | . | I | . | K | . | . | . | . | . | . | . |
| Mono- | | | | | | | | | | | | | | | | | | |
| valent | | | | | | | | | | | | | | | | | | |

LLB1 Family affinity maturation (LLB1-AM3 and LLB1-AM4)

A second round of affinity maturation using phage display was performed to increase the affinity of the LLB1 family to CD98hc (Table 11). The backbone of the library was the clone LLB1-3. Library LLB1-AM3 expands the library positions from the original clone using NHK or ARY. Library LLB1-AM4 expands the libraries and includes a mixture of residues that were found previously at those positions in the LLB1 family. The library was assembled using Kunkel mutagenesis and screened to human CD98hc. There were 20 clones selected and affinity was measured by surface plasmon resonance (SPR) using a Biacore™ machine. Sequences of the clones are shown in Table A10 and affinity of the clones is shown in Table 12A. The format was anti-BACE1 Fabs having full effector function and in a bivalent format (i.e., no knob and hole mutations). Cell binding of the clones was also tested in HeLa cells for huCD98hc binding, CHO:cyCD98hc cells for cyno binding, and CHO cells as a negative control.

TABLE 11
LLB1-AM3 and LLB2-AM4 Affinity Maturation Library

| | | 378 | 380 | 382 | 383 | 384 | 385 | 386 | 387 | 389 | |
|---|---|---|---|---|---|---|---|---|---|---|---|
| | WT Fc | A | E | E | S | N | G | Q | P | N | |
| LLB1- | AA | X | D | R | X | Y | K | P | Y | X | |
| AM3 | Codons | NHK | GAC | CGG | NHK | TAC | AAG | CCC | TAC | NHK | |
| LLB1- | AA | ASTVILF | ED | KREG | X | YENI | K | KQPT | YPFSLH | | |
| AM4 | Codons | DYM | GAN | RRR | NHK | WWY | AAG | MMR | YHY | | |
| | | 421 | 422 | 424 | 426 | 428 | 434 | 436 | 438 | 440 | 442 |
| | WT Fc | N | V | S | S | M | N | Y | Q | S | S |
| LLB1- | AA | X | I | V | D | X | NS | X | I | K | X |
| AM3 | Codons | NHK | ATC | GTG | GAC | NHK | ARY | NHK | ATC | AAG | NHK |
| LLB1- | AA | | IKVE | SVALF | DSAY | ML | NS | X | IVFL | KT | |
| AM4 | Codons | | RWA | KYN | KMY | MTG | ARY | NHK | NTY | AMR | |

TABLE A10
LLB1 library for AM3 and AM4 engineering rounds

| | 369 | 370 | 371 | 372 | 373 | 374 | 375 | 376 | 377 | 378 | 379 | 380 | 381 | 382 | 383 | 384 | 385 | 386 | 387 |
|---|---|---|---|---|---|---|---|---|---|---|---|---|---|---|---|---|---|---|---|
| WT | V | K | G | F | Y | P | S | D | I | A | V | E | W | E | S | N | G | Q | P |
| Fc | | | | | | | | | | | | | | | | | | | |
| LLB1- | . | . | . | . | . | . | . | . | . | S | . | D | . | R | . | Y | K | P | Y |
| 3-1 | | | | | | | | | | | | | | | | | | | |
| LLB1- | . | . | . | . | . | . | . | . | . | S | . | . | . | R | . | Y | K | P | Y |
| 3-2 | | | | | | | | | | | | | | | | | | | |
| LLB1- | . | . | . | . | . | . | . | . | . | V | . | . | . | R | . | Y | K | P | Y |
| 3-3 | | | | | | | | | | | | | | | | | | | |
| LLB1- | . | . | . | . | . | . | . | . | . | . | . | D | . | R | T | Y | K | P | Y |
| 3-4 | | | | | | | | | | | | | | | | | | | |
| LLB1- | . | . | . | . | . | . | . | . | . | . | . | D | . | R | . | Y | K | P | Y |
| 3-5 | | | | | | | | | | | | | | | | | | | |
| LLB1- | . | . | . | . | . | . | . | . | . | . | . | D | . | R | . | Y | K | P | Y |
| 3-6 | | | | | | | | | | | | | | | | | | | |
| LLB1- | . | . | . | . | . | . | . | . | . | . | . | D | . | R | . | Y | K | P | Y |
| 3-7 | | | | | | | | | | | | | | | | | | | |
| LLB1- | . | . | . | . | . | . | . | . | . | . | . | D | . | R | . | Y | K | P | Y |
| 3-8 | | | | | | | | | | | | | | | | | | | |
| LLB1- | . | . | . | . | . | . | . | . | . | . | . | D | . | R | . | Y | K | P | Y |
| 3-9 | | | | | | | | | | | | | | | | | | | |
| LLB1- | . | . | . | . | . | . | . | . | . | . | . | D | . | R | . | Y | K | P | Y |
| 3-10 | | | | | | | | | | | | | | | | | | | |
| LLB1- | . | . | . | . | . | . | . | . | . | . | . | D | . | R | . | Y | K | P | Y |
| 3-11 | | | | | | | | | | | | | | | | | | | |
| LLB1- | . | . | . | . | . | . | . | . | . | . | . | D | . | R | . | Y | K | P | Y |
| 3-12 | | | | | | | | | | | | | | | | | | | |
| LLB1- | . | . | . | . | . | . | . | . | . | . | . | D | . | R | . | Y | K | P | Y |
| 3-13 | | | | | | | | | | | | | | | | | | | |
| LLB1- | . | . | . | . | . | . | . | . | . | S | . | . | . | R | . | Y | K | P | Y |
| 3-14 | | | | | | | | | | | | | | | | | | | |
| LLB1- | . | . | . | . | . | . | . | . | . | . | . | D | . | R | T | Y | K | P | Y |
| 17 | | | | | | | | | | | | | | | | | | | |
| LLB1- | . | . | . | . | . | . | . | . | . | S | . | D | . | R | . | Y | K | P | Y |
| 18 | | | | | | | | | | | | | | | | | | | |
| LLB1- | . | . | . | . | . | . | . | . | . | . | . | D | . | R | T | Y | K | P | Y |
| 1-19 | | | | | | | | | | | | | | | | | | | |
| LLB1- | . | . | . | . | . | . | . | . | . | S | . | . | . | R | . | Y | K | P | Y |
| 1-20 | | | | | | | | | | | | | | | | | | | |
| LLB1- | . | . | . | . | . | . | . | . | . | V | . | . | . | R | T | Y | K | P | Y |
| 1-21 | | | | | | | | | | | | | | | | | | | |
| LLB1- | . | . | . | . | . | . | . | . | . | . | . | D | . | R | T | Y | K | P | Y |
| 1-22 | | | | | | | | | | | | | | | | | | | |

| | 388 | 389 | 390 | 391 | 392 | 418 | 419 | 420 | 421 | 422 | 423 | 424 | 425 | 426 | 427 | 428 | 429 | 430 |
|---|---|---|---|---|---|---|---|---|---|---|---|---|---|---|---|---|---|---|
| WT | E | N | N | Y | K | Q | Q | G | N | V | F | S | C | S | V | M | H | E |
| Fc | | | | | | | | | | | | | | | | | | |
| LLB1- | . | . | . | . | . | . | . | . | . | I | . | D | . | . | . | . | . | . |
| 3-1 | | | | | | | | | | | | | | | | | | |
| LLB1- | . | . | . | . | . | . | . | . | . | I | . | D | . | . | . | . | . | . |
| 3-2 | | | | | | | | | | | | | | | | | | |
| LLB1- | . | . | . | . | . | . | . | . | . | I | . | D | . | . | . | . | . | . |
| 3-3 | | | | | | | | | | | | | | | | | | |
| LLB1- | . | . | . | . | . | . | . | . | . | I | . | D | . | . | . | . | . | . |
| 3-4 | | | | | | | | | | | | | | | | | | |
| LLB1- | . | T | . | . | . | . | . | . | . | I | . | D | . | . | . | . | . | . |
| 3-5 | | | | | | | | | | | | | | | | | | |
| LLB1- | . | Y | . | . | . | . | . | . | . | I | . | D | . | . | . | . | . | . |
| 3-6 | | | | | | | | | | | | | | | | | | |
| LLB1- | . | . | . | . | . | . | . | . | D | I | . | D | . | . | . | . | . | . |
| 3-7 | | | | | | | | | | | | | | | | | | |
| LLB1- | . | . | . | . | . | . | . | . | E | I | . | D | . | . | . | . | . | . |
| 3-8 | | | | | | | | | | | | | | | | | | |
| LLB1- | . | . | . | . | . | . | . | . | . | . | . | D | . | . | . | . | . | . |
| 3-9 | | | | | | | | | | | | | | | | | | |
| LLB1- | . | . | . | . | . | . | . | . | . | I | . | D | . | L | . | . | . | . |
| 3-10 | | | | | | | | | | | | | | | | | | |
| LLB1- | . | . | . | . | . | . | . | . | . | I | . | D | . | L | . | . | . | . |
| 3-11 | | | | | | | | | | | | | | | | | | |
| LLB1- | . | . | . | . | . | . | . | . | . | I | . | D | . | Y | . | . | . | . |
| 3-12 | | | | | | | | | | | | | | | | | | |
| LLB1- | . | . | . | . | . | . | . | . | . | I | . | D | . | . | . | . | . | . |
| 3-13 | | | | | | | | | | | | | | | | | | |
| LLB1- | . | . | . | . | . | . | . | . | D | I | . | D | . | L | . | . | . | . |
| 3-14 | | | | | | | | | | | | | | | | | | |
| LLB1- | . | T | . | . | . | . | . | . | D | I | . | D | . | L | . | . | . | . |
| 17 | | | | | | | | | | | | | | | | | | |
| LLB1- | . | Y | . | . | . | . | . | . | D | I | . | D | . | L | . | . | . | . |
| 18 | | | | | | | | | | | | | | | | | | |
| LLB1- | . | F | . | . | . | . | . | . | E | I | . | D | . | L | . | . | . | . |
| 1-19 | | | | | | | | | | | | | | | | | | |
| LLB1- | . | . | . | . | . | . | . | . | . | I | . | D | . | L | . | . | . | . |
| 1-20 | | | | | | | | | | | | | | | | | | |
| LLB1- | . | . | . | . | . | . | . | . | . | I | . | D | . | L | . | . | . | . |
| 1-21 | | | | | | | | | | | | | | | | | | |
| LLB1- | . | F | . | . | . | . | . | . | Q | I | . | D | . | Y | . | . | . | . |
| 1-22 | | | | | | | | | | | | | | | | | | |

| | 431 | 432 | 433 | 434 | 435 | 436 | 437 | 438 | 439 | 440 | 441 | 442 | 443 | 444 | 445 | 446 | 447 |
|---|---|---|---|---|---|---|---|---|---|---|---|---|---|---|---|---|---|
| WT | A | L | H | N | H | Y | T | Q | K | S | L | S | L | S | P | G | K |
| Fc | | | | | | | | | | | | | | | | | |
| LLB1- | . | . | . | . | . | . | . | I | . | K | . | . | . | . | . | . | . |
| 3-1 | | | | | | | | | | | | | | | | | |
| LLB1- | . | . | . | . | . | . | . | I | . | K | . | . | . | . | . | . | . |
| 3-2 | | | | | | | | | | | | | | | | | |
| LLB1- | . | . | . | . | . | . | . | I | . | K | . | . | . | . | . | . | . |
| 3-3 | | | | | | | | | | | | | | | | | |
| LLB1- | . | . | . | . | . | . | . | I | . | K | . | . | . | . | . | . | . |
| 3-4 | | | | | | | | | | | | | | | | | |
| LLB1- | . | . | . | . | . | . | . | I | . | K | . | . | . | . | . | . | . |
| 3-5 | | | | | | | | | | | | | | | | | |
| LLB1- | . | . | . | . | . | . | . | I | . | K | . | . | . | . | . | . | . |
| 3-6 | | | | | | | | | | | | | | | | | |
| LLB1- | . | . | . | . | . | . | . | I | . | K | . | . | . | . | . | . | . |
| 3-7 | | | | | | | | | | | | | | | | | |
| LLB1- | . | . | . | . | . | . | . | I | . | K | . | . | . | . | . | . | . |
| 3-8 | | | | | | | | | | | | | | | | | |
| LLB1- | . | . | . | . | . | . | . | I | . | K | . | . | . | . | . | . | . |
| 3-9 | | | | | | | | | | | | | | | | | |
| LLB1- | . | . | . | . | . | . | . | I | . | K | . | . | . | . | . | . | . |
| 3-10 | | | | | | | | | | | | | | | | | |
| LLB1- | . | . | . | S | . | . | . | I | . | K | . | . | . | . | . | . | . |
| 3-11 | | | | | | | | | | | | | | | | | |
| LLB1- | . | . | . | . | . | . | . | I | . | K | . | . | . | . | . | . | . |
| 3-12 | | | | | | | | | | | | | | | | | |
| LLB1- | . | . | . | . | . | F | . | I | . | K | . | . | . | . | . | . | . |
| 3-13 | | | | | | | | | | | | | | | | | |
| LLB1- | . | . | . | . | . | . | . | I | . | K | . | . | . | . | . | . | . |
| 3-14 | | | | | | | | | | | | | | | | | |
| LLB1- | . | . | . | . | . | . | . | I | . | K | . | Q | . | . | . | . | . |
| 17 | | | | | | | | | | | | | | | | | |
| LLB1- | . | . | . | . | . | F | . | I | . | K | . | . | . | . | . | . | . |
| 18 | | | | | | | | | | | | | | | | | |
| LLB1- | . | . | . | . | . | F | . | I | . | K | . | . | . | . | . | . | . |
| 1-19 | | | | | | | | | | | | | | | | | |
| LLB1- | . | . | . | . | . | F | . | I | . | K | . | . | . | . | . | . | . |
| 1-20 | | | | | | | | | | | | | | | | | |
| LLB1- | . | . | . | . | . | F | . | V | . | K | . | . | . | . | . | . | . |
| 1-21 | | | | | | | | | | | | | | | | | |
| LLB1- | . | . | . | . | . | F | . | I | . | K | . | M | . | . | . | . | . |
| 1-22 | | | | | | | | | | | | | | | | | |

### LLB1 Rational Designs and PK Fix

Clone 1-3-16 was made using all the beneficial mutations for binding from previous libraries, with reversion of E380 to wild-type for improved PK. Additional clones with a reversion of E380 to wild-type were made. The sequences and affinity of these clones are shown in Table A1 l and Table 12A. Cell binding of the clones was also tested in HeLa cells for huCD98hc binding, CHO:cyCD98hc cells for cyno binding, and CHO cells as a negative control.

### LLB1 Engineering For Cyno Cross-Reactivity

In order to engineer LLB1 family to be cyno cross reactive, new libraries were designed using LLB1-3-16 clone as a template. In some libraries on the right register position 382 was kept as R or NNK, position 383 was mostly kept as S or T and in some cases changed to NNK, position 384 was alternated predominantly changed to NNK and in few cases restricted to Y, position 385 was fixed to NNK, position 386 was kept at P and occasionally changed to NNK, position 387 was randomized to NNK, position 388 was unchanged and fixed to E and position 389 was switched between N and T. On the left register position 424 was for the most part fixed to V and in some libraries was NNK and same goes with position 440 which was fixed at K and only occasionally changed to NNK. Library sizes were kept small to 1e6 and yeast display technology was used to screen them. The affinity of the resulting clones are shown in Table 12B and the sequences of the clones are shown in Table A13. The affinity of these clones was measured with non-binding Fabs having full effector function (effector+) (i.e., no modifications for modulating effector function, such as LALA or PG/PS) and in a bivalent format (i.e., no knob or hole mutations). Cell binding of the clones was also tested in HeLa cells for huCD98hc binding and CHO cells as a negative control.

TABLE 12A
Affinity measurement for LLB1 variants

| | Human CD98hc (nM) |
|---|---|
| LLB1 | Not tested |
| LLB6 | Not tested |
| LLB7 | Not tested |
| LLB1-1 | >10,000 |
| LLB1-2 | >10,000 |
| LLB1-3 | 1500 |
| LLB1-4 | No binding |
| LLB1-5 | >10,000 |
| LLB1-6 | No binding |
| LLB1-7 | No binding |
| LLB1-8 | Not tested |
| LLB1-9 | Not tested |
| LLB1-10 | Not tested |
| LLB1-11 | Not tested |
| LLB1-12 | >10,000 |
| LLB1-13 | >10,000 |
| LLB1-14 | Not tested |
| LLB1-15 | Not tested |
| LLB1-16 | Not tested |
| LLB1-3 Monovalent | 980 |
| LLB1-3-1 | 1300 |
| LLB1-3-2 | 2400 |
| LLB1-3-3 | Not determinable |
| LLB1-3-4 | 690 |
| LLB1-3-5 | 550 |
| LLB1-3-6 | 620 |
| LLB1-3-7 | 480 |
| LLB1-3-8 | 1500 |
| LLB1-3-9 | 1000 |
| LLB1-3-10 | 420 |
| LLB1-3-11 | 1200 |
| LLB1-3-12 | 1300 |
| LLB1-3-13 | 270 |
| LLB1-3-14 | 470 |
| LLB1-17 | 92 |
| LLB1-18 | 44 |
| LLB1-19 | 59 |
| LLB1-20 | 200 |
| LLB1-21 | 330 |
| LLB1-22 | 340 |
| LLB1-3 D380E | 1000 |
| LLB1-3-16 | 32 |
| LLB1-3-4-1 | 880 |
| LLB1-3-10-1 | 470 |
| LLB1-3-14-1 | 330 |
| LLB1-3-16-2 | 175 |
| LLB1-3-17-1 | Weak |
| LLB1-3-17-2 | Weak |
| LLB1-3-25-1 | 42 |
| LLB1-3-25-2 | 160 |
| LLB1-3-31-1 | Not tested |
| LLB1-3-31-2 | 1200 |

TABLE 12B
Affinity measurement for additional LLB2 variants

| | Human CD98hc (M) |
|---|---|
| LLB1-3-16.A1 | 1.55E−07 |
| LLB1-3-16.A4 | 5.34E−07 |
| LLB1-3-16.B2 | 1.54E−07 |
| LLB1-3-16.B8 | 2.07E−07 |
| LLB1-3-16.B10 | 1.84E−07 |
| LLB1-3-16.C6 | 1.78E−07 |
| LLB1-3-16.D2 | 4.31E−08 |
| LLB1-3-16.D4 | 1.80E−07 |
| LLB1-3-16.D5 | 4.03E−07 |
| LLB1-3-16.E5 | 2.61E−07 |
| LLB1-3-16.E6 | 9.50E−07 |
| LLB1-3-16.E7 | 5.33E−07 |
| LLB1-3-16.H2 | 2.91E−07 |
| LLB1-3-16.H9 | 5.20E−07 |

TABLE A11
LLB1 rational design and PK fix engineering round

| | 369 | 370 | 371 | 372 | 373 | 374 | 375 | 376 | 377 | 378 | 379 | 380 | 381 | 382 | 383 | 384 | 385 | 386 | 387 |
|---|---|---|---|---|---|---|---|---|---|---|---|---|---|---|---|---|---|---|---|
| WT Fc | V | K | G | F | Y | P | S | D | I | A | V | E | W | E | S | N | G | Q | P |
| LLB1-3 | . | . | . | . | . | . | . | . | . | . | . | . | . | R | . | Y | K | P | Y |
| D380E | | | | | | | | | | | | | | | | | | | |
| LLB1-3-16 | . | . | . | . | . | . | . | . | . | . | . | . | . | R | T | Y | K | P | Y |
| LLB1-3-4-1 | . | . | . | . | . | . | . | . | . | . | . | . | . | R | T | Y | K | P | Y |
| LLB1-3-10-1 | . | . | . | . | . | . | . | . | . | . | . | . | . | R | . | Y | K | P | Y |
| LLB1-3-14-1 | . | . | . | . | . | . | . | . | . | . | . | . | . | R | . | Y | K | P | Y |
| LLB1-3-16-2 | . | . | . | . | . | . | . | . | . | . | . | . | . | R | T | Y | K | P | Y |
| LLB1-3-17-1 | . | . | . | . | . | . | . | . | . | . | . | . | . | . | T | Y | K | P | Y |
| LLB1-3-17-2 | . | . | . | . | . | . | . | . | . | . | . | . | . | . | T | Y | K | P | Y |
| LLB1-3-25-1 | . | . | . | . | . | . | . | . | . | . | . | . | . | R | T | Y | K | P | Y |
| LLB1-3-25-2 | . | . | . | . | . | . | . | . | . | . | . | . | . | R | T | Y | K | P | Y |
| LLB1-3-31-1 | . | . | . | . | . | . | . | . | . | . | . | . | . | R | T | Y | K | P | Y |
| LLB1-3-31-2 | . | . | . | . | . | . | . | . | . | . | . | . | . | R | T | Y | K | P | Y |

| | 388 | 389 | 390 | 391 | 392 | 417 | 418 | 419 | 420 | 421 | 422 | 423 | 424 | 425 | 426 | 427 | 428 | 429 |
|---|---|---|---|---|---|---|---|---|---|---|---|---|---|---|---|---|---|---|
| WT Fc | E | N | N | Y | K | W | Q | Q | G | N | V | F | S | C | S | V | M | H |
| LLB1-3 | . | . | . | . | . | . | . | . | . | . | I | . | V | . | D | . | . | . |
| D380E | | | | | | | | | | | | | | | | | | |
| LLB1-3-16 | . | T | . | . | . | . | . | . | . | D | I | . | V | . | D | . | L | . |
| LLB1-3-4-1 | . | . | . | . | . | . | . | . | . | . | I | . | V | . | D | . | . | . |
| LLB1-3-10-1 | . | . | . | . | . | . | . | . | . | . | I | . | V | . | D | . | L | . |
| LLB1-3-14-1 | . | . | . | . | . | . | . | . | . | D | I | . | V | . | D | . | L | . |
| LLB1-3-16-2 | . | T | . | . | . | . | . | . | . | D | I | . | V | . | D | . | . | . |
| LLB1-3-17-1 | . | T | . | . | . | . | . | . | . | D | I | . | V | . | D | . | L | . |
| LLB1-3-17-2 | . | T | . | . | . | . | . | . | . | D | I | . | V | . | D | . | . | . |
| LLB1-3-25-1 | . | T | . | . | . | . | . | . | . | D | I | . | V | . | D | . | L | . |
| LLB1-3-25-2 | . | T | . | . | . | . | . | . | . | D | I | . | V | . | D | . | . | . |
| LLB1-3-31-1 | . | T | . | . | . | . | . | . | . | D | I | . | V | . | D | . | L | . |
| LLB1-3-31-2 | . | T | . | . | . | . | . | . | . | D | I | . | V | . | D | . | . | . |

| | 430 | 431 | 432 | 433 | 434 | 435 | 436 | 437 | 438 | 439 | 440 | 441 | 442 | 443 | 444 | 445 | 446 | 447 |
|---|---|---|---|---|---|---|---|---|---|---|---|---|---|---|---|---|---|---|
| WT Fc | E | A | L | H | N | H | Y | T | Q | K | S | L | S | L | S | P | G | K |
| LLB1-3 | . | . | . | . | . | . | . | . | I | . | K | . | . | . | . | . | . | . |
| D380E | | | | | | | | | | | | | | | | | | |
| LLB1-3-16 | . | . | . | . | . | . | F | . | I | . | K | . | . | . | . | . | . | . |
| LLB1-3-4-1 | . | . | . | . | . | . | . | . | I | . | K | . | . | . | . | . | . | . |
| LLB1-3-10-1 | . | . | . | . | . | . | . | . | I | . | K | . | . | . | . | . | . | . |
| LLB1-3-14-1 | . | . | . | . | . | . | . | . | I | . | K | . | . | . | . | . | . | . |
| LLB1-3-16-2 | . | . | . | . | . | . | F | . | I | . | K | . | . | . | . | . | . | . |
| LLB1-3-17-1 | . | . | . | . | . | . | F | . | I | . | K | . | . | . | . | . | . | . |
| LLB1-3-17-2 | . | . | . | . | . | . | F | . | I | . | K | . | . | . | . | . | . | . |
| LLB1-3-25-1 | . | . | . | . | . | . | F | . | I | . | K | . | . | . | . | . | . | . |
| LLB1-3-25-2 | . | . | . | . | . | . | F | . | I | . | K | . | . | . | . | . | . | . |
| LLB1-3-31-1 | . | . | . | . | . | . | F | . | I | . | . | . | . | . | . | . | . | . |
| LLB1-3-31-2 | . | . | . | . | . | . | F | . | I | . | . | . | . | . | . | . | . | . |

TABLE A13
LLB1 library engineering for cyno cross-reactivity

| | 369 | 370 | 371 | 372 | 373 | 374 | 375 | 376 | 377 | 378 | 379 |
|---|---|---|---|---|---|---|---|---|---|---|---|
| WT Fc | V | K | G | F | Y | P | S | D | I | A | V |
| LLB1-1-3-16 | | | | | | | | | | | |
| LLB1-3-16.A1 | | | | | | | | | | | |
| LLB1-3-16.A4 | | | | | | | | | | | |
| LLB1-3-16.B2 | | | | | | | | | | | |
| LLB1-3-16.B8 | | | | | | | | | | | |
| LLB1-3-16.B10 | | | | | | | | | | | |
| LLB1-3-16.C6 | | | | | | | | | | | |
| LLB1-3-16.D2 | | | | | | | | | | | |
| LLB1-3-16.D4 | | | | | | | | | | | |
| LLB1-3-16.D5 | | | | | | | | | | | |
| LLB1-3-16.E5 | | | | | | | | | | | |
| LLB1-3-16.E6 | | | | | | | | | | | |
| LLB1-3-16.E7 | | | | | | | | | | | |
| LLB1-3-16.H2 | | | | | | | | | | | |
| LLB1-3-16.H9 | | | | | | | | | | | |

| | 380 | 381 | 382 | 383 | 384 | 385 | 386 | 387 | 388 | 389 |
|---|---|---|---|---|---|---|---|---|---|---|
| WT Fc | E | W | E | S | N | G | Q | P | E | N |
| LLB1-1-3-16 | | | R | T | Y | K | P | Y | | T |
| LLB1-3-16.A1 | | | R | T | H | K | P | Y | | T |
| LLB1-3-16.A4 | | | R | T | Y | K | P | H | | F |
| LLB1-3-16.B2 | | | R | | Y | K | P | L | | R |
| LLB1-3-16.B8 | | | R | | F | R | P | Y | | |
| LLB1-3-16.B10 | | | R | | Y | S | P | H | | T |
| LLB1-3-16.C6 | | | R | | Y | R | P | Y | | |
| LLB1-3-16.D2 | | | R | | Y | K | P | Y | | T |
| LLB1-3-16.D4 | | | R | | H | K | P | Y | | T |
| LLB1-3-16.D5 | | | R | | H | R | P | Y | | T |
| LLB1-3-16.E5 | | | R | T | Y | R | P | F | | T |
| LLB1-3-16.E6 | | | R | | F | R | P | Y | | |
| LLB1-3-16.E7 | | | R | | Y | R | P | Y | | |
| LLB1-3-16.H2 | | | R | | Y | K | P | L | | T |
| LLB1-3-16.H9 | | | R | | Y | K | P | Y | | |

| | 421 | 422 | 423 | 424 | 425 | 426 | 427 | 428 | 429 | 430 | 431 |
|---|---|---|---|---|---|---|---|---|---|---|---|
| WT Fc | N | V | F | S | C | S | V | M | H | E | A |
| LLB1-1-3-16 | D | I | | V | | D | | L | | | |
| LLB1-3-16.A1 | D | I | | V | | D | | L | | | |
| LLB1-3-16.A4 | D | I | | V | | D | | L | | | |
| LLB1-3-16.B2 | D | I | | V | | D | | L | | | |
| LLB1-3-16.B8 | D | I | | V | | D | | L | | | |
| LLB1-3-16.B10 | D | I | | V | | D | | L | | | |
| LLB1-3-16.C6 | D | I | | L | | D | | L | | | |
| LLB1-3-16.D2 | D | I | | V | | D | | L | | | |
| LLB1-3-16.D4 | D | I | | V | | D | | L | | | |
| LLB1-3-16.D5 | D | I | | V | | D | | L | | | |
| LLB1-3-16.E5 | D | I | | V | | D | | L | | | |
| LLB1-3-16.E6 | D | I | | I | | D | | L | | | |
| LLB1-3-16.E7 | D | I | | V | | D | | L | | | |
| LLB1-3-16.H2 | D | I | | V | | D | | L | | | |
| LLB1-3-16.H9 | D | I | | V | | D | | L | | | |

| | 432 | 433 | 434 | 435 | 436 | 437 | 438 | 439 | 440 | 441 |
|---|---|---|---|---|---|---|---|---|---|---|
| WT Fc | L | H | N | H | Y | T | Q | K | S | L |
| LLB1-1-3-16 | | | | | F | | I | | K | |
| LLB1-3-16.A1 | | | | | F | | I | | K | |
| LLB1-3-16.A4 | | | | | F | | I | | K | |
| LLB1-3-16.B2 | | | | | F | | I | | K | |
| LLB1-3-16.B8 | | | | | F | | I | | K | |
| LLB1-3-16.B10 | | | | | F | | I | | K | |
| LLB1-3-16.C6 | | | | | F | | I | | K | |
| LLB1-3-16.D2 | | | | | F | | I | | K | |
| LLB1-3-16.D4 | | | | | F | | I | | K | |
| LLB1-3-16.D5 | | | | | F | | I | | K | |
| LLB1-3-16.E5 | | | | | F | | I | | K | |
| LLB1-3-16.E6 | | | | | F | | I | | K | |
| LLB1-3-16.E7 | | | | | F | | I | | T | |
| LLB1-3-16.H2 | | | | | F | | I | | K | |
| LLB1-3-16.H9 | | | | | F | | I | | | |

### Example 4. Plasma PK of LLB2 and LLB1 CD98HC Binders

To begin characterizing the CD98hc-binding molecules, pharmacokinetics (PK) was assessed in in wild-type mice to demonstrate in vivo stability in a model lacking CD98hc-mediated clearance, as these CD98hc-binding molecules only bind human CD98hc and not murine CD98hc. The study design is shown in Table 13 below. 6-8 week-old C57B 16 (WT) mice were intravenously dosed and in-life bleeds were taken via submandibular-bleeds, at time points as indicated in Table 13 below. Blood was collected in EDTA plasma tubes, spun at 14,000 rpm for 5 minutes, and then plasma was isolated for subsequent analysis.

TABLE 13
Study Design

| | | CD98hc | | | | | Dose |
|---|---|---|---|---|---|---|---|
| | | binding | | EF | Time | | IV |
| Clone # | Clone name | valency | Fabs | status | points | N | (mg/kg) |
| Ctrl 1 | Non-binding | — | NBF | + | 0.5 h, 1 d, | 3 | 10 |
| | Fab | | | | 4 d, 7 d | | |
| | (negative | | | | | | |
| | control) | | | | | | |
| Clone 4 | LLB1-3 | Bivalent | NBF | + | 0.5 h, 1 d, | 3 | 10 |
| | D380E | | | | 4 d, 7 d | | |
| Clone 6 | LLB1-3 | Mono- | NBF | + | 0.5 h, 1 d, | 3 | 10 |
| | D380E | valent | | | 4 d, 7 d | | |
| Clone 8 | LLB2-10-6 | Mono- | NBF | + | 0.5 h, 1 d, | 3 | 10 |
| | | valent | | | 4 d, 7 d | | |
| Clone 9 | LLB2-10-8 | Mono- | NBF | + | 0.5 h, 1 d, | 3 | 10 |
| | | valent | | | 4 d, 7 d | | |
| Clone | LLB2-10-28 | Mono- | NBF | + | 0.5 h, 1 d, | 3 | 10 |
| 12 | | valent | | | 4 d, 7 d | | |

The total huIgG concentrations in plasma were quantified using a generic anti-human IgG sandwich-format ELISA. Briefly, plates were coated overnight at 4 C with donkey anti-human IgG (JIR #709-006-098) at 1 μg/mL in sodium bicarbonate solution (Sigma #C3041-50CAP) with gentle agitation. Plates were then washed 3× with wash buffer (PBS+0.0500 Tween 20). Assay standards and samples were diluted in PBS+0.05% Tween 20 and 100 BSA (10 mg/mL). Standard curve preparation ranged from 0.41 to 1,500 ng/mL or 0.003 to 10 nM (BLQ<0.03 nM). Standards and diluted samples were incubated with agitation for 2 hr at room temperature. After incubation, plates were washed 3× with wash buffer. Detection antibody, goat anti-human IgG(JIR #109-036-098), was diluted in blocking buffer (PBS+0.05% Tween 20+500 BSA (50 mg/mL)) to a final concentration of 0.02 μg/mL and plates were incubated with agitation for 1 hr at room temperature. After a final 3× wash, plates were developed by adding TMB substrate and incubated for 5-10 minutes. Reaction was quenched by adding 4N H₂SO₄ and read using 450 nM absorbance.

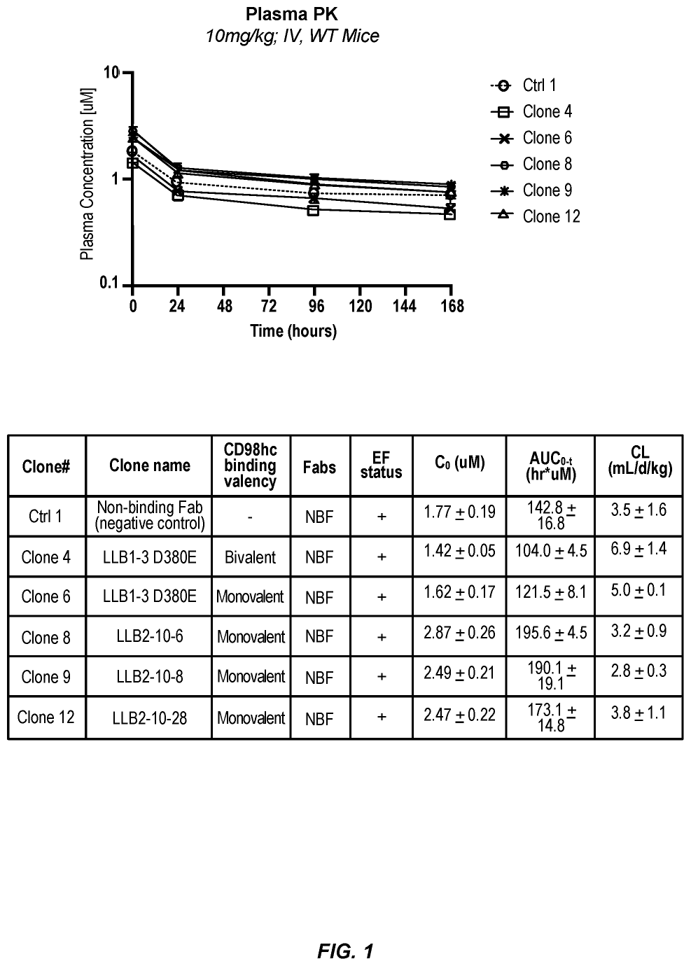

Results are shown in FIG. 1. All molecules exhibited similar clearance values as the control IgG.

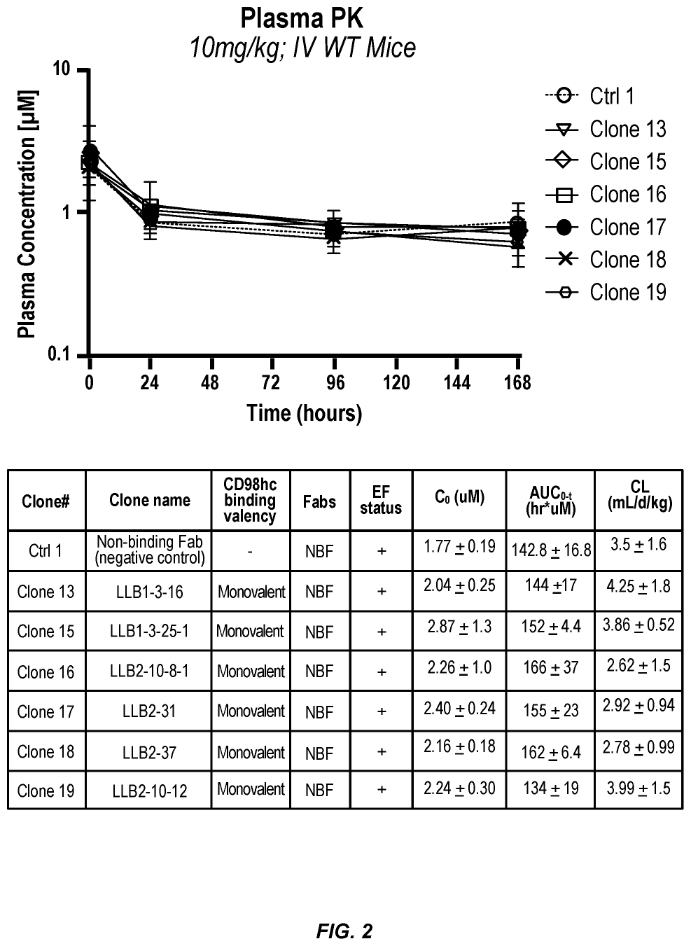

Pharmacokinetics (PK) were tested for additional LLB1 and LLB2 clones in WT mice according to the study design in Table 14. Pharmacokinetics (PK) were assessed following the protocol for sample collection and analysis as described above. Results are shown in FIG. 2. The additional LLB1 and LLB2 clones tested exhibited similar clearance values as the control IgG.

TABLE 14
Study Design

| | | CD98hc | | | | | Dose |
|---|---|---|---|---|---|---|---|
| | Clone | binding | | EF | Time | | IV |
| Clone # | name | valency | Fabs | status | points | N | (mg/kg) |
| Ctrl 1 | Non-binding | — | NBF | + | 0.5 h, 1 d, | 3 | 10 |
| | Fab | | | | 4 d, 7 d | | |
| | (negative | | | | | | |
| | control) | | | | | | |
| Clone | LLB1-3-16 | Mono- | NBF | + | 0.5 h, 1 d, | 3 | 10 |
| 13 | | valent | | | 4 d, 7 d | | |
| Clone | LLB1-3-25-1 | Mono- | NBF | + | 0.5 h, 1 d, | 3 | 10 |
| 15 | | valent | | | 4 d, 7 d | | |
| Clone | LLB2-10-8-1 | Mono- | NBF | + | 0.5 h, 1 d, | 3 | 10 |
| 16 | | valent | | | 4 d, 7 d | | |
| Clone | LLB2-31 | Mono- | NBF | + | 0.5 h, 1 d, | 3 | 10 |
| 17 | | valent | | | 4 d, 7 d | | |
| Clone | LLB2-37 | Mono- | NBF | + | 0.5 h, 1 d, | 3 | 10 |
| 18 | | valent | | | 4 d, 7 d | | |
| Clone | LLB2-10-12 | Mono- | NBF | + | 0.5 h, 1 d, | 3 | 10 |
| 19 | | valent | | | 4 d, 7 d | | |

Pharmacokinetics (PK) were assessed following the protocol for sample collection and analysis as described above. Results are shown in FIG. 2. All molecules exhibited similar clearance values as the control IgG.

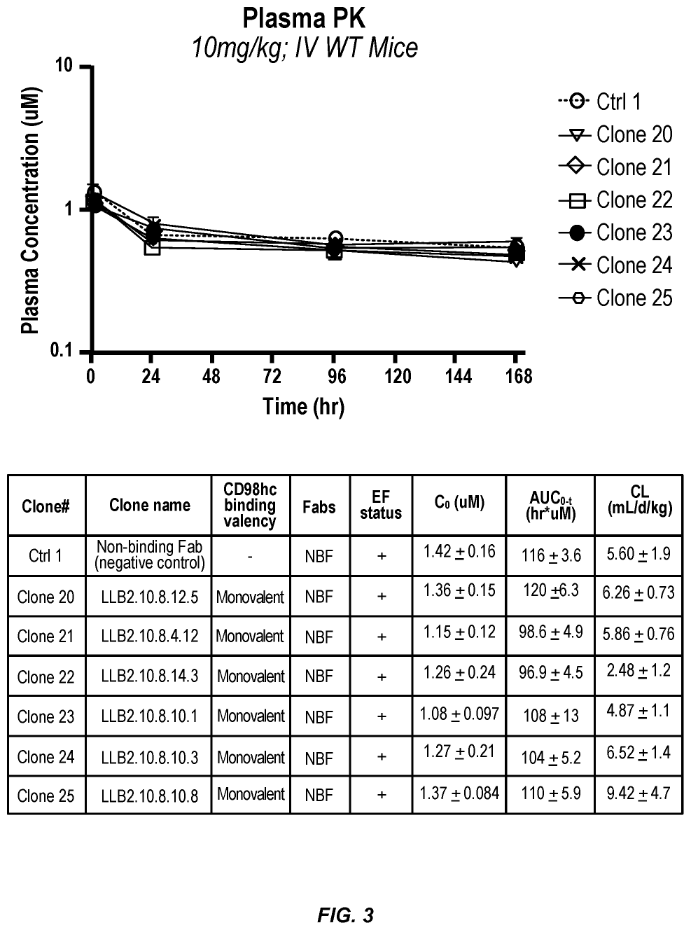

Pharmacokinetics (PK) were tested for affinity matured LLB2 clones (i.e., stronger binding to CD98hc) in WT mice according to the study design in Table 15. Pharmacokinetics (PK) were assessed following the protocol for sample collection and analysis as described above. Results are shown in FIG. 3. All affinity matured LLB2 clones tested exhibited similar clearance values as the control IgG.

TABLE 15
Study Design

| | | | | | | | Dose |
|---|---|---|---|---|---|---|---|
| | | CD98hc | | | | | IV |
| Clone | Clone | binding | | EF | Time | | (mg/ |
| # | name | valency | Fabs | status | points | N | kg) |
| Ctrl 1 | Non-binding | — | NBF | + | 0.5 h, 1 d, | 3 | 10 |
| | Fab | | | | 4 d, 7 d | | |
| | (negative | | | | | | |
| | control) | | | | | | |
| Clone | LLB2.10.8.12.5 | Mono- | NBF | + | 0.5 h, 1 d, | 3 | 10 |
| 20 | | valent | | | 4 d, 7 d | | |
| Clone | LLB2.10.8.4.12 | Mono- | NBF | + | 0.5 h, 1 d, | 3 | 10 |
| 21 | | valent | | | 4 d, 7 d | | |
| Clone | LLB2.10.8.14.3 | Mono- | NBF | + | 0.5 h, 1 d, | 3 | 10 |
| 22 | | valent | | | 4 d, 7 d | | |
| Clone | LLB2.10.8.10.1 | Mono- | NBF | + | 0.5 h, 1 d, | 3 | 10 |
| 23 | | valent | | | 4 d, 7 d | | |
| Clone | LLB2.10.8.10.3 | Mono- | NBF | + | 0.5 h, 1 d, | 3 | 10 |
| 24 | | valent | | | 4 d, 7 d | | |
| Clone | LLB2.10.8.10.8 | Mono- | NBF | + | 0.5 h, 1 d, | 3 | 10 |
| 25 | | valent | | | 4 d, 7 d | | |

Pharmacokinetics (PK) were assessed following the protocol for sample collection and analysis as described above. Results are shown in FIG. 3. All molecules exhibited similar clearance values as the control IgG.

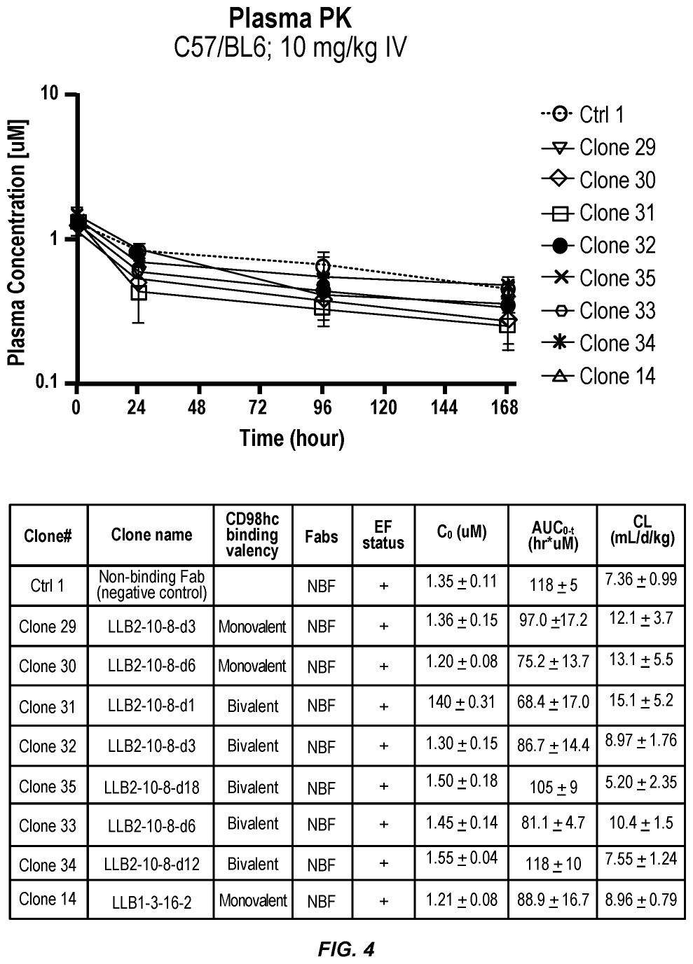

Pharmacokinetics (PK) were tested for de-affinity matured LLB2 clones (i.e., weaker binding to CD98hc) in WT mice according to the study design in Table 16. Pharmacokinetics (PK) were assessed following the protocol for sample collection and analysis as described above. Results are shown in FIG. 4. All de-affinity matured LLB2 clones tested exhibited similar clearance values as the control IgG.

TABLE 16
Study Design

| | | CD98hc | | | | | Dose |
|---|---|---|---|---|---|---|---|
| Clone | Clone | binding | | EF | Time | | IV |
| # | name | valency | Fabs | status | points | N | (mg/kg) |
| Ctrl 1 | Non-binding | — | NBF | + | 0.5 h, 1 d, | 3 | 10 |
| | Fab | | | | 4 d, 7 d | | |
| | (negative | | | | | | |
| | control) | | | | | | |
| Clone | LLB2-10-8-d3 | Mono- | NBF | + | 0.5 h, 1 d, | 3 | 10 |
| 29 | | valent | | | 4 d, 7 d | | |
| Clone | LLB2-10-8-d6 | Mono- | NBF | + | 0.5 h, 1 d, | 3 | 10 |
| 30 | | valent | | | 4 d, 7 d | | |
| Clone | LLB2-10-8-d1 | Bivalent | NBF | + | 0.5 h, 1 d, | 3 | 10 |
| 31 | | | | | 4 d, 7 d | | |
| Clone | LLB2-10-8-d3 | Bivalent | NBF | + | 0.5 h, 1 d, | 3 | 10 |
| 32 | | | | | 4 d, 7 d | | |
| Clone | LLB2-10-8-d18 | Bivalent | NBF | + | 0.5 h, 1 d, | 3 | 10 |
| 35 | | | | | 4 d, 7 d | | |
| Clone | LLB2-10-8-d6 | Bivalent | NBF | + | 0.5 h, 1 d, | 3 | 10 |
| 33 | | | | | 4 d, 7 d | | |
| Clone | LLB2-10-8-d12 | Bivalent | NBF | + | 0.5 h, 1 d, | 3 | 10 |
| 34 | | | | | 4 d, 7 d | | |
| Clone | LLB1-3-16-2 | Mono- | NBF | + | 0.5 h, 1 d, | 3 | 10 |
| 14 | | valent | | | 4 d, 7 d | | |

Pharmacokinetics (PK) were assessed following the protocol for sample collection and analysis as described above. Results are shown in FIG. 4. All molecules exhibited similar clearance values as the control IgG.

### Example 5. Brain Uptake of LLB2 and LLB1 CD98HC Binders

Next, to characterize the CD98hc dependent brain uptake of LLB32 and LLB1 CD98hc-binding molecules, 6-month old homozygous CD98hc^{m/hu} KI mice were intravenously dosed 50 mg/kg according to. the study design in Table 17 below.

A full ECD knock-in CD98hc mouse model (i.e., CD98hc^{mu/hu} KI or SLCA3A2^{huECD/huECD}) was designed and generated for this study. The construct for humanizing the extracellular domain (ECD) of CD98hc contained 5 primary elements. First, 3′ and 5′ arms homologous to the endogenous mouse SLC3A2 locus to enable homologous recombination. Next, point mutations were made in murine exons 2, 3, and 4 to humanize only those extracellular mouse residues that differ from the orthologous human residues. This second element enabled preservation of mouse introns 1, 2, and 3, which are predicted to have promoter regulatory regions, and the endogenous splice sites at the intron1-exonl/2, intron2-exon 2/3, intron3-exon 3/4, and intron4-exon4 junctions. The third element was an FRT flanked Neo cassette (neomycin resistance gene) into murine intron 4, which served to disrupt a long region of mouse homology that could have caused incomplete incorporation of the entire construct and enabled screening for partial incorporation based on neomycin antibiotic resistance. Because intron 4 also contained predicted promoter regulatory regions we were concerned the Neo cassette could disrupt Slc3a2 expression therefore the FRT sites provided the option to remove the cassette in ES after incorporation was confirmed. The fourth element was the cDNA of human residues E335 to the STOP codon in place of the murine genomic DNA from residue S268 in exon 5 to the STOP codon in exon 10, which achieved humanization of the remainder of the CD98hc ECD while providing enough differentiation from the endogenous mouse sequence that homologous recombination of the entire construct could be achieved. The fifth element was a F3′ flanked hygro cassette (hygromycin resistance gene) downstream of the murine 3′ UTR, which enabled screening for incorporation of the entire construct by adding hygromycin in addition to neomycin to the ES cell culture medium.

Because the hygromycin was after the stop codon, we did not anticipate it disrupting Slc3a2 expression and the cassette would be automatically excised in the male germline of the resulting mice. This construct was electroporated into ES cells from C57B16 mice. ES cells with proper homologous recombination were selected for by growing the cells in the presence of neomycin and hygromycin. Incorporation was confirmed by PCR. The Neo cassette was removed in vitro by electroporation of a Flp recombinase expressing construct. This step was critical as the Neo cassette was suspected to disrupt expression of the SLC3A2 gene, and CD98hc protein is required for sperm function. This approach resulted in surface expression of hCD98hc on the ES cells. By contrast, ES cells that retained the Neo cassette did not express huCD98hc on ES cells. ES cells containing the properly incorporated humanized SLC3A2 gene without the Neo cassette were injected into goGermline blastocytes (Ozgene), followed by embryo transfer to pseudo pregnant females. Founder males were selected from the offspring of the female that received the embryos and bred to wild-type females to generate F1 heterozygous mice. Homozygous mice were subsequently generated from breeding of F1 generation heterozygous mice.

TABLE 17
Study Design

| | | CD98hc | | | | | | | |
|---|---|---|---|---|---|---|---|---|---|
| | | binding | | EF | huCD98hc | | Terminal | | Dose IV |
| Clone # | Clone name | valency | Fabs | status | Affinity | | time point | N | (mg/kg) |
| Ctrl 1 | Non-binding Fab | — | NBF | + | — | | 2 d | 5 | 50 |
| | (negative | | | | | | | | |
| | control) | | | | | | | | |
| Clone 8 | LLB2-10-6 | Monovalent | NBF | + | 230 | nM | 2 d | 5 | 50 |
| Clone 9 | LLB2-10-8 | Monovalent | NBF | + | 100-200 | nM | 2 d | 5 | 50 |
| Clone 12 | LLB2-10-28 | Monovalent | NBF | + | 150 | nM | 2 d | 5 | 50 |
| Clone 1 | LLB2-10-8 | Bivalent | BACE1 | + | 100-200 | nM | 2 d | 5 | 50 |
| Clone 4 | LLB1-3 D380E | Bivalent | NBF | + | 1 | uM | 2 d | 5 | 50 |
| Ctrl 2 | CD98/BACE1 | Monovalent | — | + | 80 | nm | 2 d | 5 | 50 |
| | (+control mAb) | | | | | | | | |

After 48 hours from dosing, blood was collected via cardiac puncture, and the mice were perfused with PBS. Brain tissue was homogenized using a Qiagen TissueLyser in 10× tissue weight of lysis buffer containing 1% NP-40 in PBS with protease inhibitors. Blood was collected in EDTA tubes to prevent clotting and spun at 14000 rpm for 7 minutes to isolate plasma. Brain samples were homogenized in 1% NP40 lysis buffer and lysates diluted 1:2 and 1:20 for PK analysis. huIgG was measured, as described above, using a generic anti-human IgG sandwich-format ELISA. Dosing solutions were also analyzed on the same plate to confirm the correct dosage.

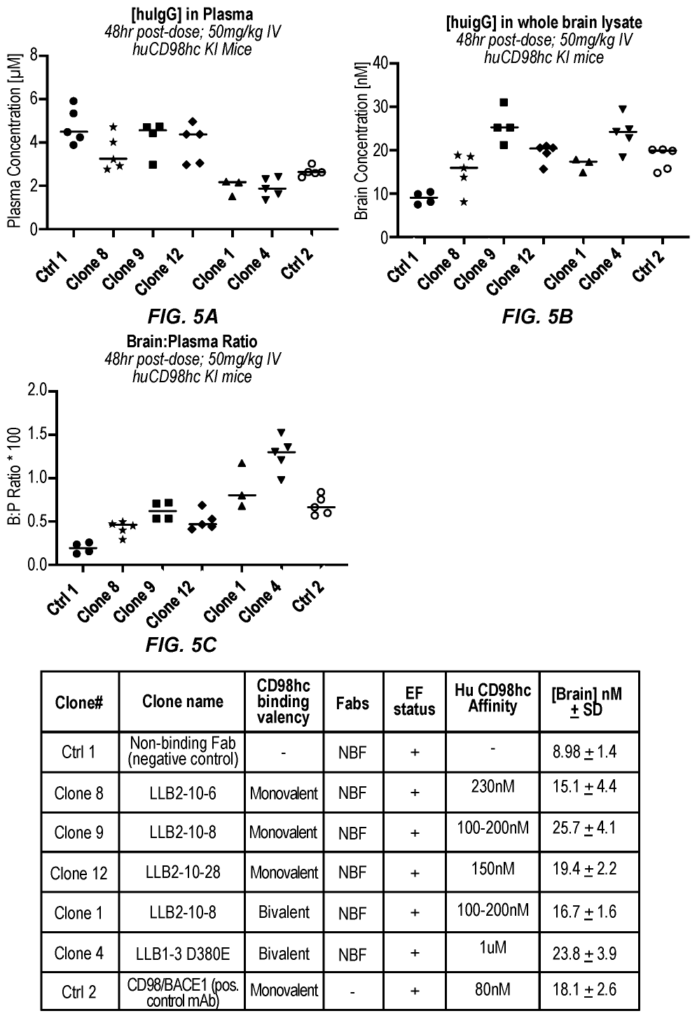

huIgG levels in plasma and brain 48 hr post 50 mg/kg IV dose of LLB1 and LLB2 variants in CD98hc^{mu/hu}KI mice are shown in FIGS. 5A-5C. After 48 hours from dosing, the plasma levels of CD98hc-binding molecules were lower than the levels for Ctrl 1, likely due to clearance of this antibody via binding to peripherally-expressed huCD98hc. The positive control anti-CD98hc/BACE1 antibody and the bivalent LLB2 and LLB1 clones were present in plasma at reduced concentrations, likely due to target-mediated drug disposition (TMDD) (FIG. 5A). In whole brain, a 2-3-fold increase in the concentration of CD98hc binding molecules compared to Ctrl 1 was observed (FIG. 5B). The ratio of huIgG in brain to plasma is shown in FIG. 5C. All CD98hc-binding molecules had higher brain to plasma ratios compared to the control molecule, and bivalent LLB1-3 D380E had the highest. The significant accumulation of the CD98hc-binding molecules in the brain parenchyma is due to CD98hc mediated transcytosis at the BBB.

After perfusion with PBS, brains were dissected, and the meninges and choroid plexus removed. The fresh brain was homogenized with a Dounce homogenizer in HBSS. Homogenized samples were centrifuged (1,000 g for 10 min). After homogenization, an aliquot of the supernatant (non-cell associated fraction) was taken. Cell pellets were resuspended in 17% dextran. An additional aliquot of the total spun down cells (to represent all cells: cell-associated) was collected, washed, and lysed in lysis buffer containing 1% NP-40 in PBS with protease inhibitors. The remaining resuspended cells were centrifuged at 4,122 g for 15 min. Resulting cell pellet contained vasculature and the supernatant contained parenchymal cells. Supernatant was added to a tube containing 10 mL of HBSS and spun at 4,122 g for 15 min. Cell pellet contained parenchymal cells. Both vascular and parenchymal cell pellets were resuspended in lysis buffer containing 1% NP-40 in PBS with protease inhibitors. Total protein concentrations of samples were measured using BCA. huIgG concentration was measured as described above using human IgG assay (a generic anti-human IgG sandwich-format ELISA) and then normalized to total protein concentration in the sample.

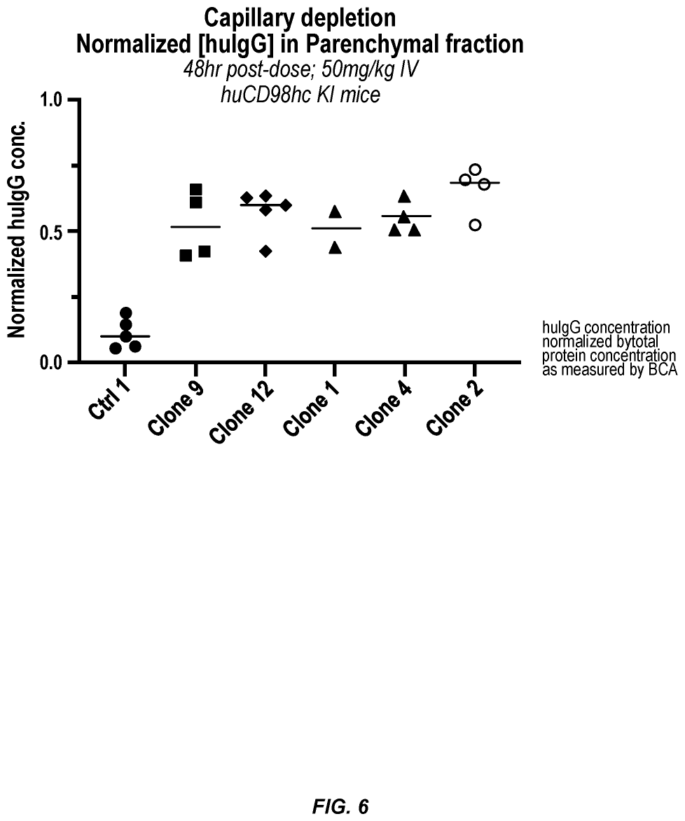

In the parenchymal fraction, an 5-8-fold increase in the concentration of all CD98hc-binding molecules was observed compared to Ctrl 1 (negative control molecule with non-binding Fab (NBF)), demonstrating that CD98hc-binding molecules cross the BBB into the brain parenchyma. All huIgG values were normalized to total protein concentration measured by BCA (FIG. 6).

### Example 6. CNS Biodistribution of LLB2 and LLB1 Cd98Hc Binders

To characterize LLB2 and LLB1 CNS biodistribution, IHC for huIgG was performed to determine the cell type specific localization of 3 clones selected from Example 5 above: Clone 4 (bivalent LLB1 clone), 9 (monovalent LLB2 clone), and 12 (monovalent LLB2 clone). After perfusion with PBS, hemi-brains were drop fixed in 4% PFA overnight. Sagittal brain sections (40 μm) were cut using a microtome, blocked in 5% BSA+0.3% Triton X-100, followed by fluorescent staining with Alexa488 anti-huIgG (Jackson Immunoresearch 109-545-003, 1:500), rabbit anti-Iba1 (Abcam abl78846, 1:500) or rabbit anti-aquaporrin4 (Millipore AB2218, 1:500)+goat anti-rabbit-568 (Invitrogen A-11011, 1:500). Brain images were taken using a Leica SP8 Lightning confocal microscope with a 40× objective. Broad brain vasculature and parenchymal staining was observed for CD98hc-binding molecules.

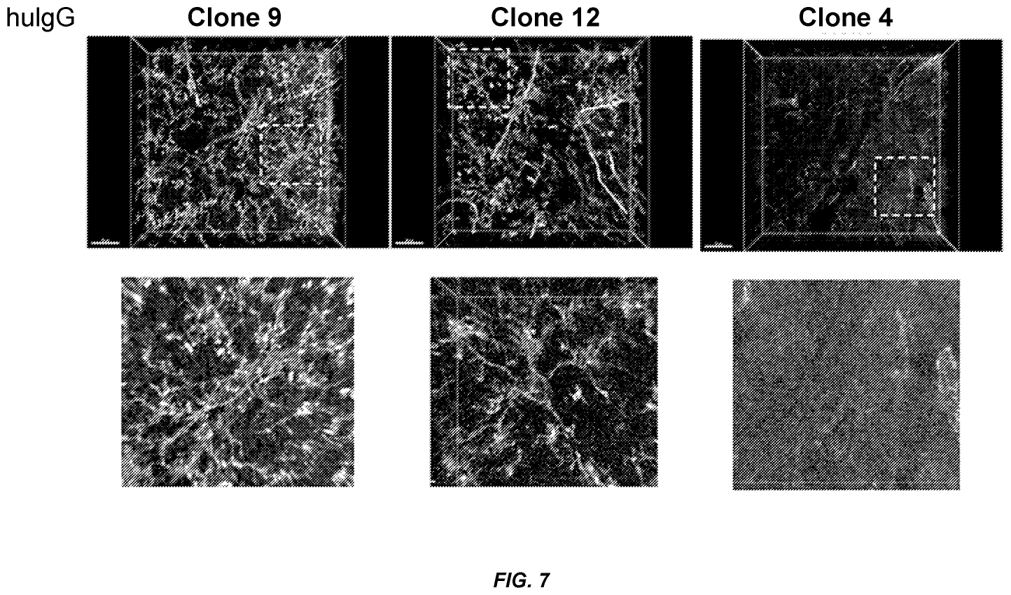

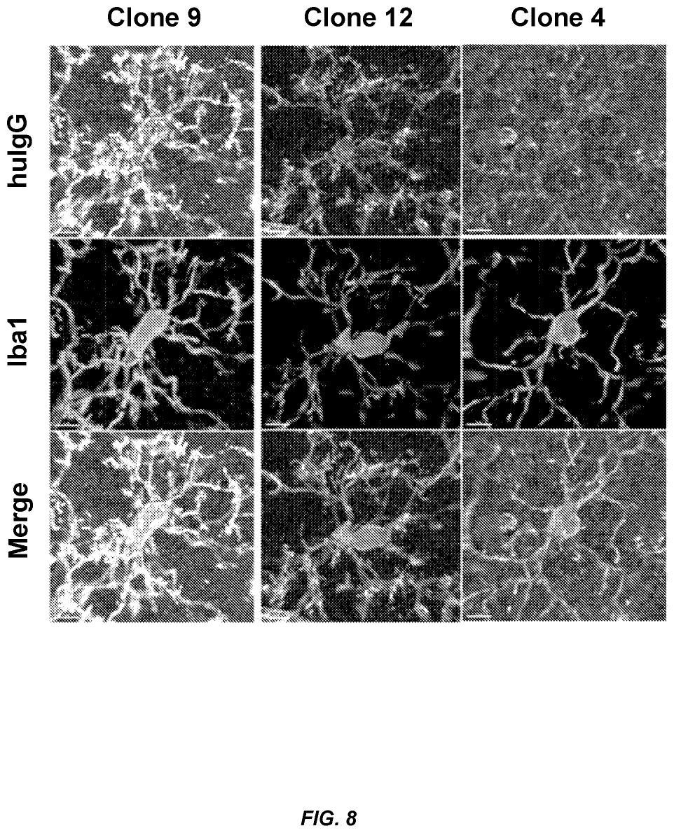

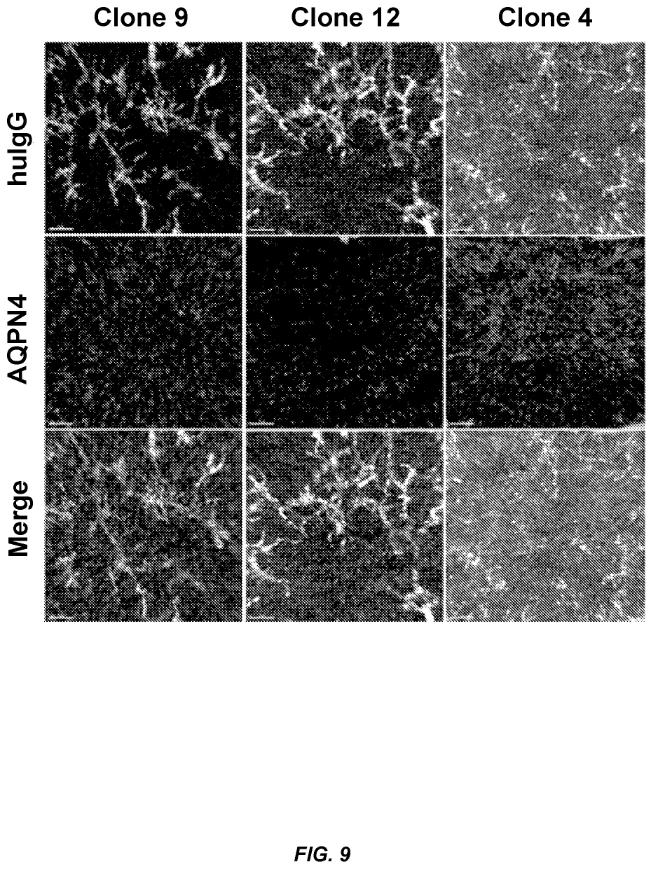

Immunohistochemistry for huIgG, huIgG and Iba1 (microglia marker), and huIgG and AQP4 (marker of astrocyte processes and endfeet) on brain sections from CD98hc^{mu/hu} KI mice 48 hr post 50 mpk dose of LLB2 and LLB1 molecules are shown in FIGS. 7-9. LLB2 molecules prominently localize to microglia whereas LLB1 molecules more weakly localize to microglia and have a prominent diffuse punctate staining (FIG. 7). Anti-huIgG signal for the 2 monovalent LLB2 molecules (Clones 9 and 12) colocalizes with Iba1, consistent with monovalent LLB2 molecules localizing to microglia. Anti-huIgG for the bivalent LLB1 molecule (Clone 4) also co-localizes with Iba1 but more weakly than LLB2 (FIG. 8). The diffuse punctate staining of the bivalent LLB1 molecule robustly colocalizes with AQP4, indicating that LLB1 molecules localize to astrocyte processes, consistent with CD98hc expression (FIG. 9). Therefore, different CD98hc binding families have distinct biodistribution in the CNS.

### Example 7. Brain Uptake and Peripheral Tissue Localization of LLB2 and LLB1 Variants

Brain uptake of additional variants of LLB2 and valency matched LLB1 CD98hc-binding molecules were tested in homozygous CD98hc^{mu/hu} KI mice. The study design is shown in Table 18 below. 2-4 month old homozygous CD98hc^{mu/hu}KI mice were intravenously dosed 50 mg/kg.

TABLE 18
Study Design

| | | CD98hc | | | | | | |
|---|---|---|---|---|---|---|---|---|
| | | binding | | EF | huCD98hc | Terminal | | Dose IV |
| Clone # | Clone name | valency | Fabs | status | Affinity | time point | N | (mg/kg) |
| Ctrl 1 | Non-binding | — | NBF | + | — | 2 d | 5 | 50 |
| | Fab (negative | | | | | | | |
| | control) | | | | | | | |
| Clone 19 | LLB2-10-12 | Monovalent | NBF | + | 130 | 2 d | 5 | 50 |
| Clone 9 | LLB2-10-8 | Monovalent | NBF | + | 100-200 | 2 d | 5 | 50 |
| Clone 8 | LLB2-10-6 | Monovalent | NBF | + | 260 | 2 d | 5 | 50 |
| Clone 17 | LLB2-31 | Monovalent | NBF | + | 270 | 2 d | 5 | 50 |
| Clone 13 | LLB1-3-16 | Monovalent | NBF | + | 32 | 2 d | 5 | 50 |

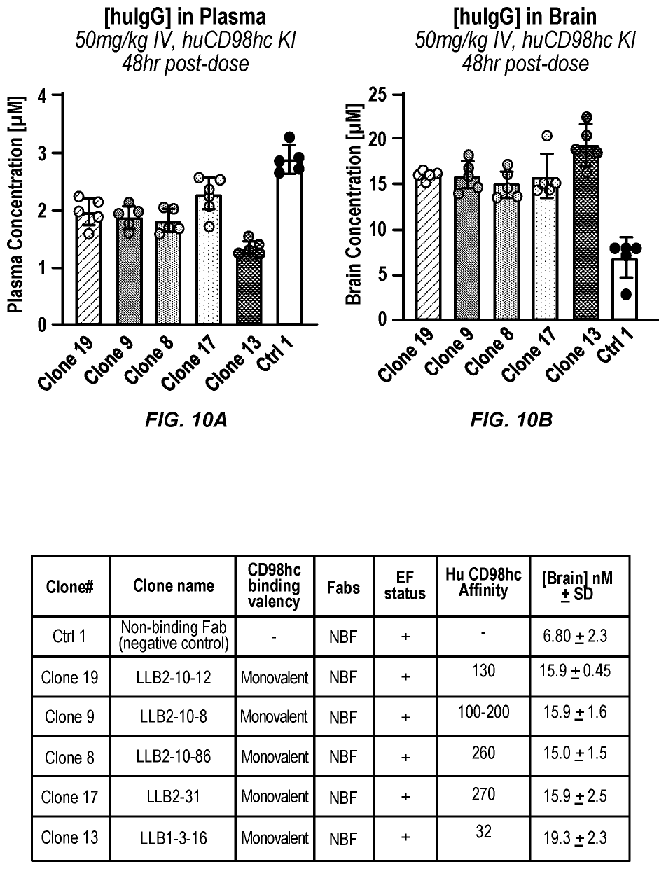

huIgG concentrations in plasma and brain 48 hr post 50 mg/kg IV dose of additional LLB1 and LLB2 variants in CD98hc^{mu/hu} KI mice mice were assessed as described above and results are shown in FIGS. 10A and 10B. Plasma concentrations of CD98hc-binding molecules were lower that the Ctrl 1 and the higher affinity LLB1 clone was the lowest (FIG. 10A). Increased concentrations were observed for all huCD98hc-binding molecules compared to a control IgG. The higher affinity LLB1 also had the highest concentration in brain (FIG. 10B).

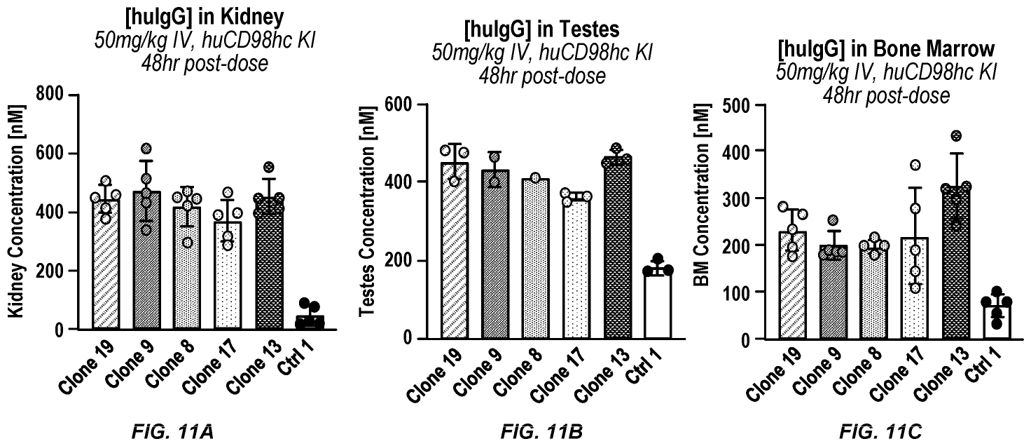

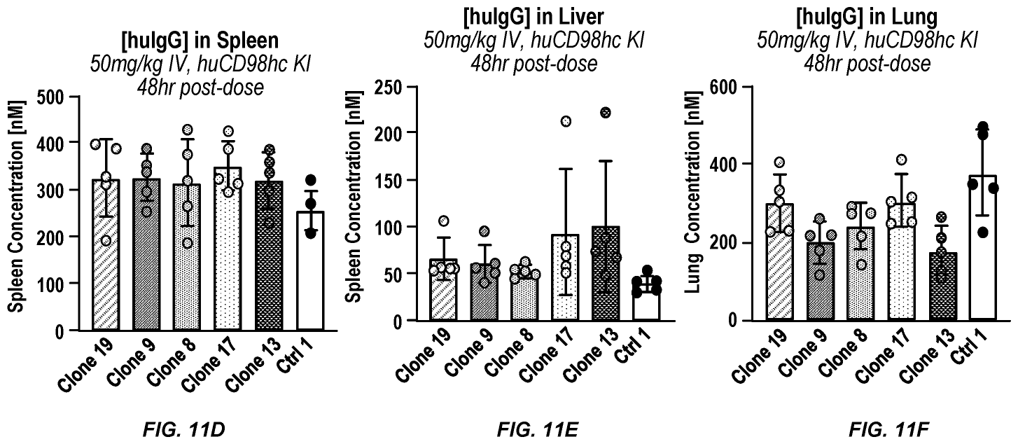

Concentrations of huIgG in peripheral tissues (plasma, kidney, testis, bone marrow, lung, liver) were also measured (FIGS. 11A-11F). Kidney and testis express high levels of CD98hc, bone marrow has intermediate expression levels, spleen has low expression levels, and lung and liver do not express CD98hc. CD98hc^{mu/hu}KI mice were dosed 50 mpk with LLB2 and LLB1 variants and huIgG concentrations were assessed 48 hr post dose. LLB2 and LLB1 variants localized to peripheral tissues consistent with CD98hc expression. Highest concentrations (relative to control IgG) of LLB2 and LLB1 molecules were observed in kidney and testis, and lower concentrations but still 2-3 fold over control were measured in bone marrow. In spleen, liver, and lung LLB2 and LLB1 concentrations were either slightly more or equivalent to control IgG.

### Example 8. Plasma and Brain PK of Monvalent and Bivalent LLB2 CD98HC BINDERS

To further characterize LLB2 molecules, plasma and brain exposure of the molecules over time was assessed in plasma, brain, kidney, testis, pancreas, lung, liver, spleen, intestine, and bone marrow. The study design is shown in Table 19 below. 6-8 month old homozygous CD98hc^{mu/hu} KI mice were intravenously dosed 50 mg/kg according to the groups in Table 19 and plasma, brain, and peripheral tissues were collected at 1, 2, 4, 7, and 10 days post dose.

TABLE 19
Study Design

| | | CD98hc | | | | Terminal | | Dose |
|---|---|---|---|---|---|---|---|---|
| | Clone | binding | | EF | Affinity | time points | | IV |
| Clone # | Name | valency | Fabs | status | (nM) | (days) | N | (mg/kg) |
| Clone 8 | LLB2-10-6 | Monovalent | NBF | + | 260 | 1, 2, 4, 7, | 5 per | 50 |
| | | | | | | and 10 | timepoint | |
| Clone 9 | LLB2-10-8 | Monovalent | NBF | + | 100-200 | 1, 2, 4, 7, | 5 per | 50 |
| | | | | | | and 10 | timepoint | |
| Clone 26 | LLB2-10-8 | Bivalent | NBF | + | 100-200 | 1, 2, 4, 7, | 5 per | 50 |
| | | | | | | and 10 | timepoint | |
| Clone 18 | LLB2-37 | Monovalent | NBF | + | 390 | 1, 2, 4, 7, | 5 per | 50 |
| | | | | | | and 10 | timepoint | |
| Clone 28 | LLB2-37 | Bivalent | NBF | + | 390 | 1, 2, 4, 7, | 5 per | 50 |
| | | | | | | and 10 | timepoint | |
| Ctrl 1 | Non-binding | — | NBF | + | N/A | 1, 2, 4, 7, | 5 per | 50 |
| | Fab | | | | | | | |
| | (negative | | | | | | | |
| | control) | | | | | | | |

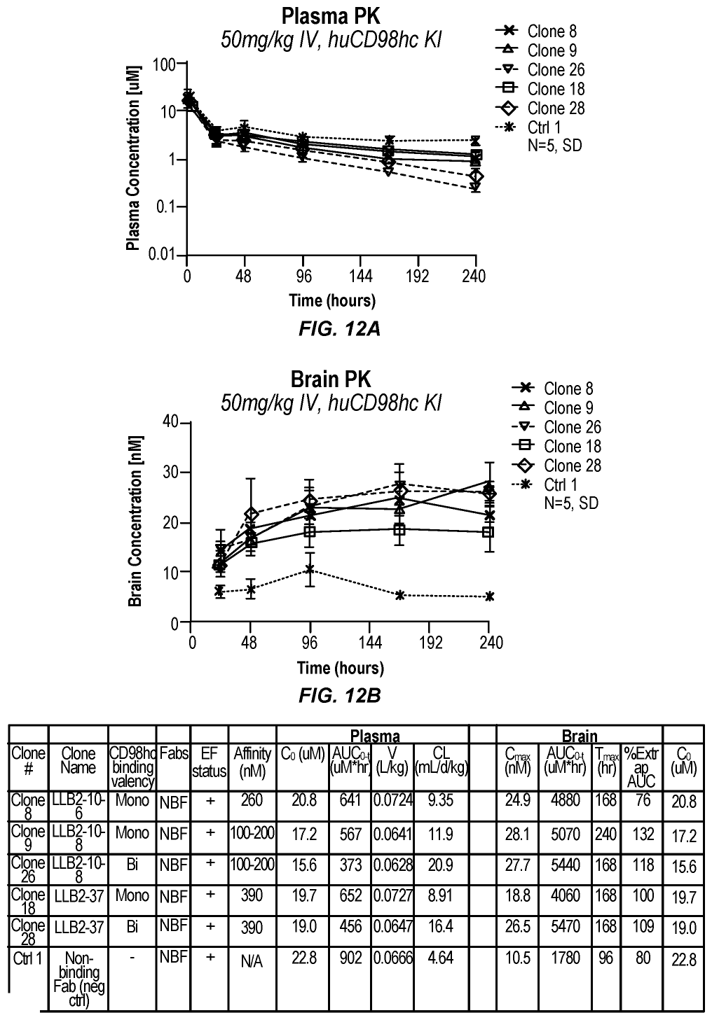

Concentrations of huIgG were assessed as described above. huIgG pharmacokinetics (PK) in plasma and brain post 50 mg/kg IV dose of monovalent and bivalent LLB2 variants in CD98hc^{mu/hu} KI mice are shown in FIGS. 12A and 12B. CD98hc-binding molecules clear faster in plasma than the control IgG (FIG. 12A). Clearance values for monovalent LLB2 were 9-12 mL/d/kg whereas bivalent molecules cleared faster: 16-21 mL/d/kg. Increased huIgG concentrations were observed for all huCD98hc-binding molecules compared to a control IgG in brain (FIG. 12B). Brain concentrations of monovalent and bivalent LLB2 generally increased until 7 days post dose and levels remained elevated at 10 days post dose. Therefore, the kinetics of CD98hc binding molecule brain uptake appears slower and more prolonged than has been observed in TfR binding molecules (i.e., Tₘₐₓ of roughly 24-48 hours post dose and concentrations in brain drop off sooner). huIgG concentrations in brain were similar for the tested CD98hc-binding molecules except for the weaker affinity monovalent clone (LLB2-37), which was consistently lower.

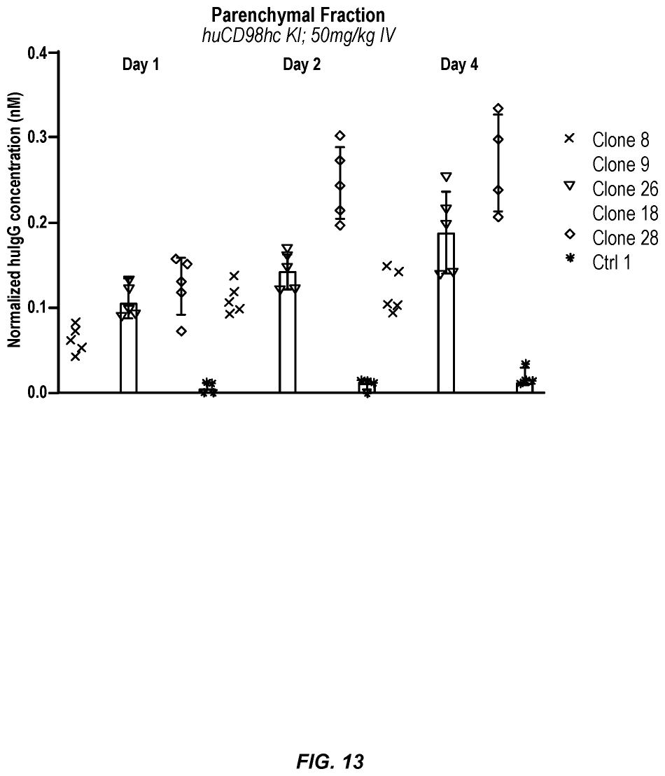

Furthermore, capillary depletion demonstrated that monovalent and bivalent LLB2 variants cross the BBB into the brain parenchyma (FIG. 13). There were increased concentrations of all CD98hc-binding molecules in the brain parenchyma compared to the negative control molecule. huIgG concentration in the parenchyma also generally increased over time. All huIgG values were normalized to total protein concentration measured by BCA.

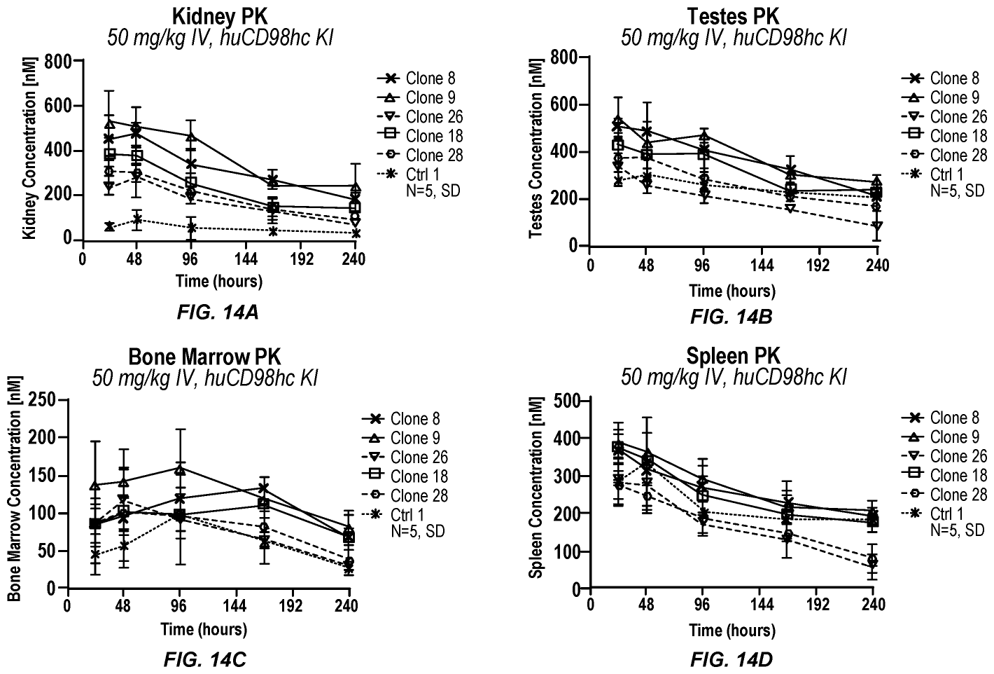

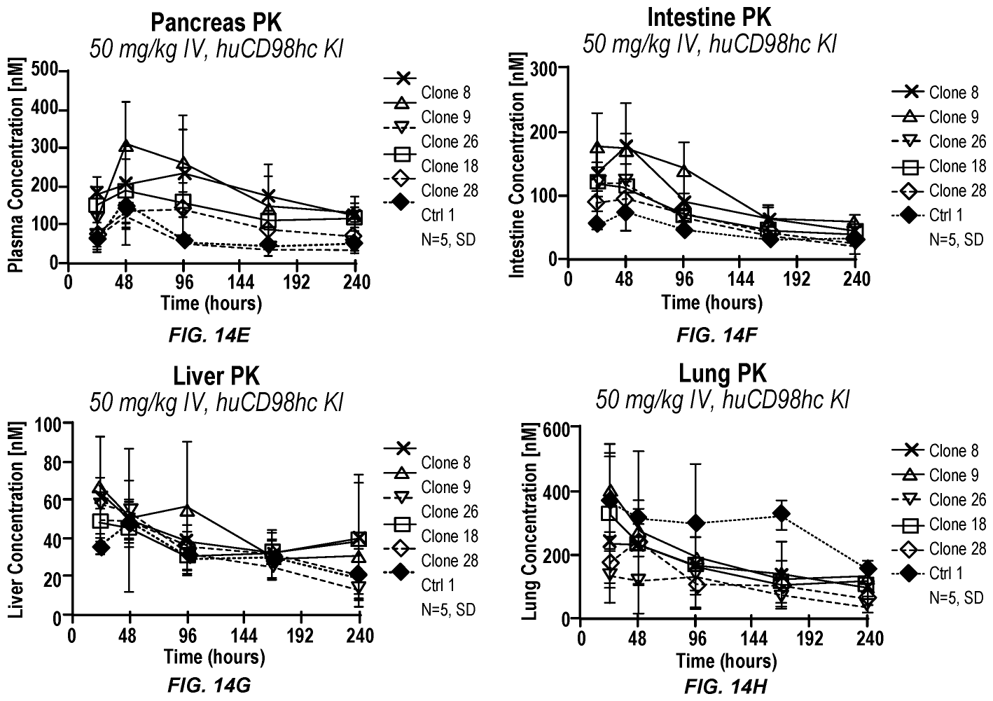

huIgG pharmacokinetics (PK) in peripheral tissues with varying expression levels of CD98hc post 50 mg/kg IV dose of monovalent and bivalent LLB2 variants in CD98hc^{mu/hu} KI mice are shown in FIGS. 14A-14H. Kidney, testis, and pancreas express high levels of CD98hc, bone marrow and intestines have intermediate expression levels, spleen has low expression levels, and lung and liver do not express CD98hc. Monovalent and bivalent LLB2 variants localized to peripheral tissues consistent with CD98hc expression. Highest concentrations (relative to control IgG) of LLB2 molecules were observed in kidney, testis, and pancreas, and lower concentrations but still 2-3 fold over control were measured in bone marrow. In spleen, liver, and lung LLB2 concentrations were either similar or lower than control IgG. There were higher concentrations of monovalent clones in tissues with high CD98hc expression, which was likely driven by higher plasma concentration of the monovalent clones. Unlike brain, concentrations of LLB2 clones decreased in all tested peripheral tissues over the course of the study.

### Example 9. Biodistribution Timecourse of Monovalent and Bivalent LLB2-10⁻⁸

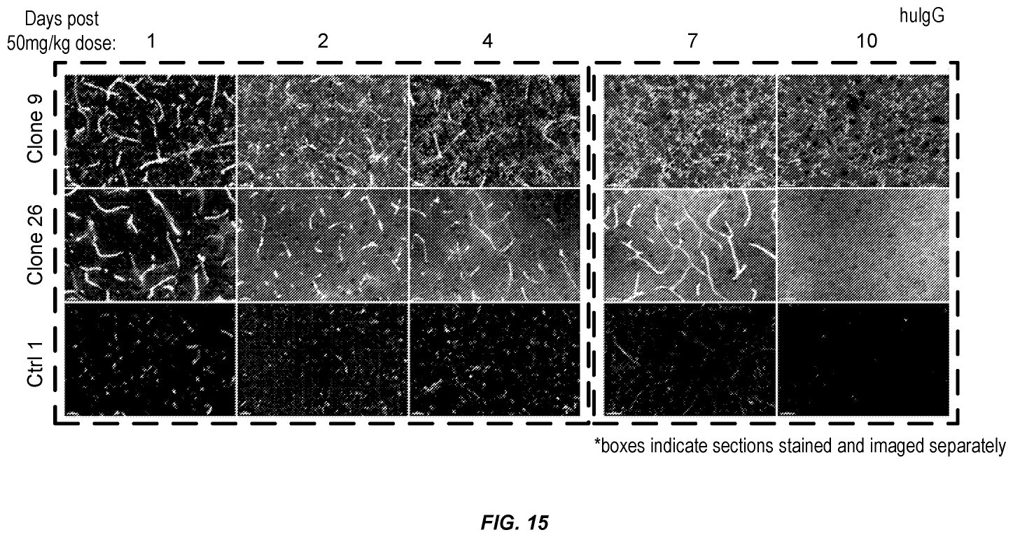

To assess the biodistribution of monovalent and bivalent LLB2-10-8 molecules over time, immunohistochemistry for huIgG on brain sections from CD98hc^{mu/hu}KI mice 1, 2, 4, 7, and 10 days post 50 mpk dose of monovalent LLB2-9-10 (Clone 9) and bivalent LLB2-10-8 (Clone 26) was performed. Fixed brain was collected and stained as described above. Results are shown in FIG. 15. There is an increase in parenchymal huIgG staining at early time points, consistent with the huIgG ELISA quantification on whole brain lysate and the parenchymal capillary depletion fraction for all molecules. Broad brain vasculature and parenchymal staining was observed for CD98hc-binding molecules. Monovalent LLB2 (Clone 9) prominently localizes to microglia whereas bivalent LLB2 (Clone 26) has a prominent diffuse punctate staining, that as shown in FIG. 9 co-localizes with astrocyte processes (AQP4). CD98hc localization to astrocytes is consistent with CD98hc expression. Therefore, monovalent and bivalent LLB2 variants appear to have have a distinct CNS biodistribution.

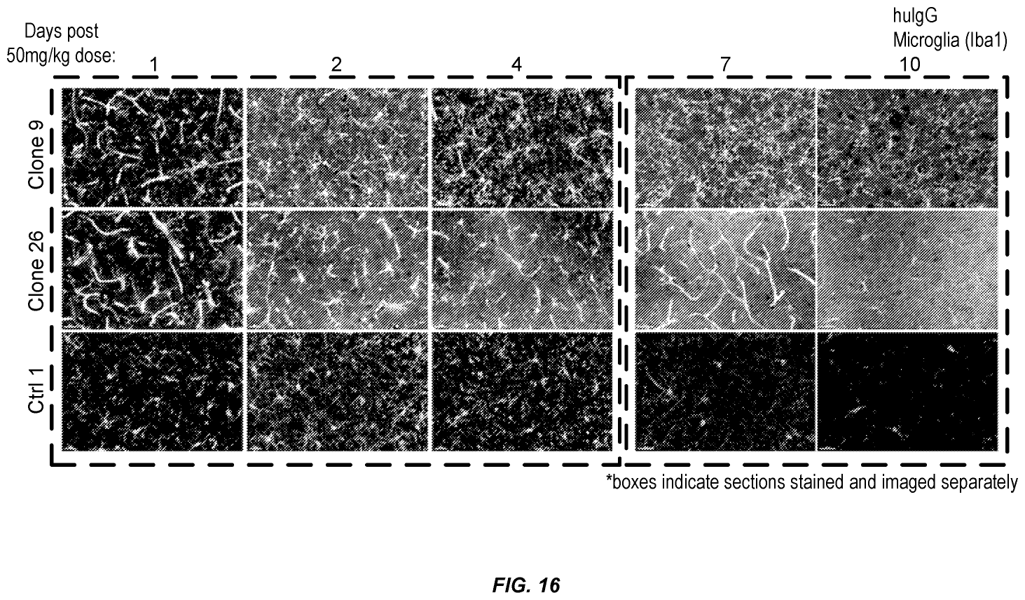

Results for immunohistochemistry for huIgG and Iba1 (microglia) on brain sections from CD98hc^{mu/hu}KI mice 1, 2, 4, 7, and 10 days post 50 mpk dose of monovalent and bivalent LLB2-10-8 are shown in FIG. 16. Monovalent LLB2 (Clone 9) robustly co-localizes with Iba1 whereas bivalent LLB2 (Clone 26) minimally co-localizes with Iba1. Therefore, monovalent CD98hc binding appears to be required for microglial localization of LLB2 variants.

### Example 10. Plasma and Brain Exposure and Biodistribution after Repeat Dosing of Monvalent and Bivalent LLB2 Variants

To study the effect of chronic dosing on safety and transport capacity over time, plasma and brain exposure was assessed after repeat dosing of 50 mg/kg. Additionally, differences in LLB2 molecules with (i.e., effector positive) and without (i.e., effector negative via LALAPG mutation) effector function was analyzed. The study design is shown in Tables 20A and 20B below. 2-4 month old homozygous CD98hc^{mu/hu} KI mice were intravenously dosed 50 mg/kg weekly for 4 weeks (5 doses). huIgG in plasma was measured 30 minutes and 6 days (C^{max} and C_{trough}) after each first 3 doses. After the 4th dose only Cₘₐₓ sample was collected. Animals were taken down 24 hrs after the 5^{th} dose and terminal plasma was collected. Brain, blood, and peripheral tissues were also collected 24 hours after the 5^{th} dose.

TABLE 20A
Monovalent LLB2 Study Design

| | | CD98hc | | | | Terminal | | |
|---|---|---|---|---|---|---|---|---|
| | Clone | binding | | EF | Affinity | time | | Dose IV |
| Clone # | Name | valency | Fabs | status | (nM) | points | N | (mg/kg) |
| Clone 8 | LLB2-10-6 | Monovalent | NBF | + | 260 | 24 hr after | 5 | 50 mg/kg |
| | | | | | | final dose | | weekly for |
| | | | | | | | | 4 weeks |
| Clone 9 | LLB2-10-8 | Monovalent | NBF | + | 100-200 | 24 hr after | 5 | 50 mg/kg |
| | | | | | | final dose | | weekly for |
| | | | | | | | | 4 weeks |
| Clone 27 | LLB2-10-8 | Monovalent | NBF | LALAPG | 100-200 | 24 hr after | 5 | 50 mg/kg |
| | | | | | | final dose | | weekly for |
| | | | | | | | | 4 weeks |
| Ctrl 1 | Non-binding | — | NBF | + | N/A | 24 hr after | 5 | 50 mg/kg |
| | Fab | | | | | final dose | | weekly for |
| | (negative | | | | | | | 4 weeks |
| | control) | | | | | | | |

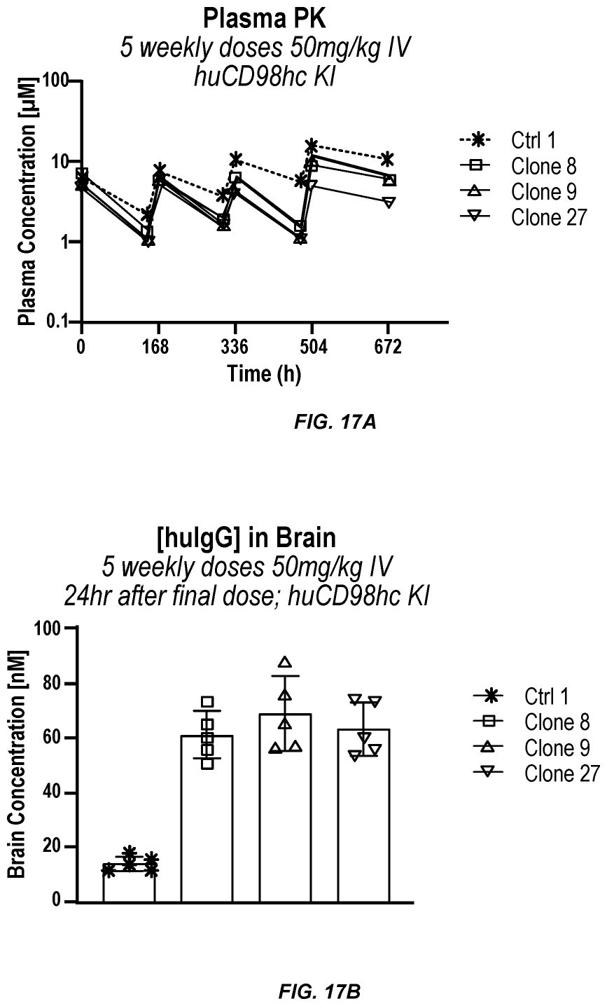

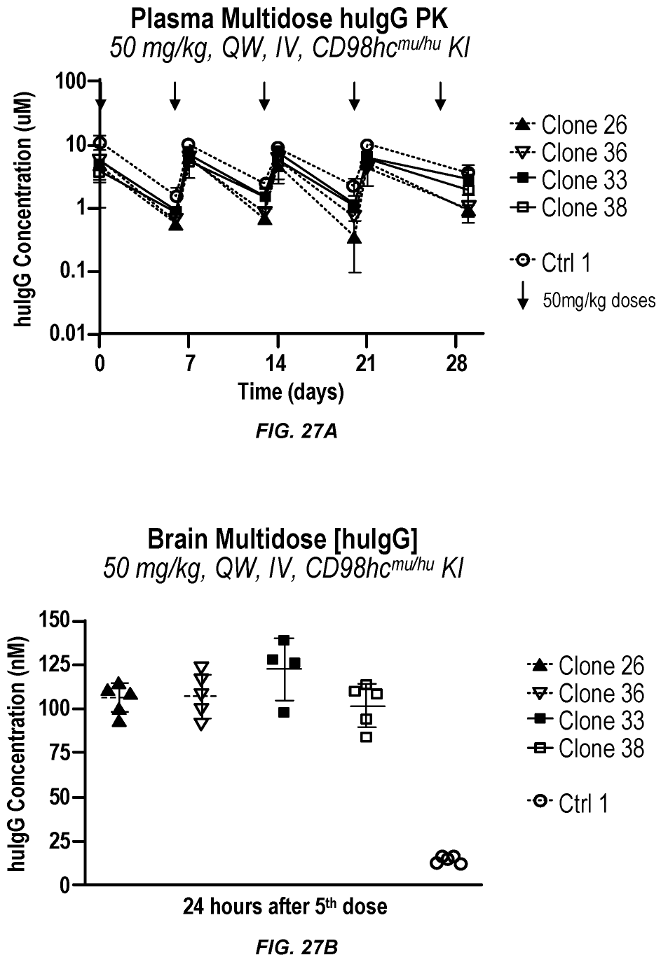

Plasma and brain exposure after repeat dosing of monovalent LLB2 variants are shown in FIGS. 17A and 17B. Plasma concentrations of monovalent LLB2 variants were lower than the control IgG (FIG. 17A). huIgG in brain was assessed 24 hrs after the 5^{th} dose. Compared to single dose studies (i.e., FIGS. 12A and 12B), there was accumulation of LLB2 monovalent variants in brain after repeat dosing (FIG. 27B). Additionally, no significant findings were observed in histopathology of peripheral tissues or in hematology or clinical chemistry. For all monovalent CD98hc TVs, reticulocyte, lymphocyte, or monocyte cell numbers were not impacted regardless of effector function.

Plasma and brain exposure after repeat dosing of bivalent LLB2 variants are shown in FIGS. 27A and 27B. Plasma concentrations of bivalent LLB2 variants were lower than the control IgG (FIG. 27A). huIgG in brain was assessed 24 hrs after the 5^{th} dose and compared to single dose studies (i.e., FIGS. 12A and 12B, 32A-32F, 34A-34F) there was accumulation of LLB2 bivalent variants in brain after repeat dosing. For all bivalent CD98hc TVs, reticulocyte, lymphocyte, or monocyte cell numbers were not impacted regardless of effector function. This safety profile means that CD98hc-binding molecules, with either monovalent or bivalent binding, could be used for targets that would ideally be targeted by therapeutics retaining wildtype effector function.

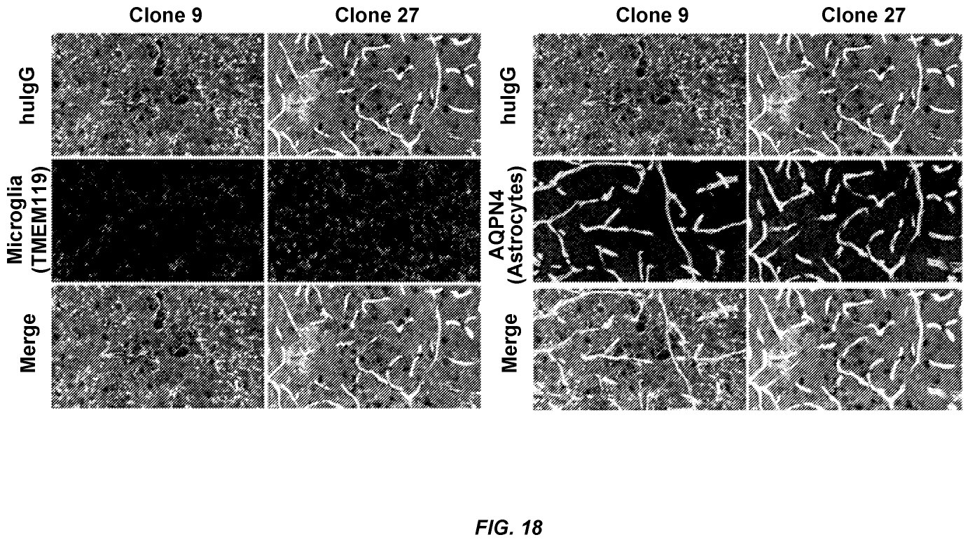

Furthermore, fixed brain was collected and stained as described above. Immunohistochemistry for huIgG on brain sections from CD98hc^{mu/hu} KI mice mice dosed weekly with 50 mpk for 4 weeks with monovalent LLB2-10-8 variants with either WT Fc (i.e., effector positive) and LALAPG mutations to make the Fc unable to bind FcγRs (i.e., effector negative) is shown in FIG. 18. Broad brain vasculature and parenchymal staining was observed for CD98hc-binding molecules. LLB2-10-8 that can bind FcγRs (Clone 9) robustly localizes to microglia, as shown by co-localization with TMEM119 (a cell surface microglia marker) and astrocyte processes as shown by co-localization with AQP4. LLB2-10-8 without effector function (Clone 27) minimally co-localizes with TMEM119, but co-localizes with AQP4. These data indicate that effector function is required for monovalent LLB2 variants to localize to microglia, i.e., effector function influences the biodistribution of these molecules in the CNS. These data suggests that for monovalent LLB2 CD98hc-binding molecules, binding to FcγRs on microglia can, at least partially, override CD98hc driven localization to astrocytes.

TABLE 20B
Bivalent LLB2 Study Design

| | | CD98hc | | | | Terminal | | |
|---|---|---|---|---|---|---|---|---|
| | Clone | binding | | EF | Affinity | time | | Dose IV |
| Clone # | Name | valency | Fabs | status | (nM) | points | N | (mg/kg) |
| Clone 26 | LLB2-10-8 | Bivalent | NBF | + | 100-200 | 24 hr after | 5 | 50 mg/kg |
| | | | | | | final dose | | weekly for |
| | | | | | | | | 4 weeks |
| Clone 36 | LLB2-10-8 | Bivalent | NBF | − | 100-200 | 24 hr after | 5 | 50 mg/kg |
| | | | | | | final dose | | weekly for |
| | | | | | | | | 4 weeks |
| Clone 33 | LLB2-10-8 | Bivalent | NBF | + | 550 | 24 hr after | 5 | 50 mg/kg |
| | d6 | | | | | final dose | | weekly for |
| | | | | | | | | 4 weeks |
| Clone 38 | LLB2-10-8 - | Bivalent | NBF | − | 550 | 24 hr after | 5 | 50 mg/kg |
| | d6 | | | | | final dose | | weekly for |
| | | | | | | | | 4 weeks |
| Ctrl 1 | Non-binding | — | NBF | + | N/A | 24 hr after | 5 | 50 mg/kg |
| | Fab | | | | | final dose | | weekly for |
| | (negative | | | | | | | 4 weeks |
| | control) | | | | | | | |

### Example 11. Design and Characterization of Engineered TFR-Binding Polypeptides Based on a Beta-Sheet Library

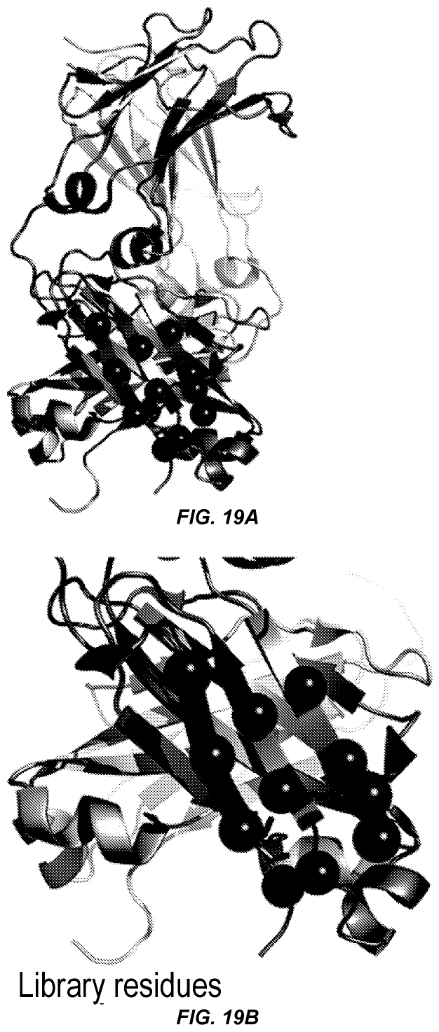

We designed a library (termed 6.5.11) with diversity in residues predominantly located on the solvent-exposed side of the CH3 domain beta-sheet surface. This region has many advantages. For example, the beta-sheet structure is stable and should allow for amino acid diversity without destabilizing the CH3 domain fold. Moreover, a library based on the 6.5.11 register does not involve large randomization of flexible loop regions that may introduce undesired conformational flexibility. The beta-sheet region also forms a concave surface that may be ideal for protein-protein interactions. This concave surface is distinct from the FcRn and FcγR binding surface. The library is mapped onto the structure of the Fc polypeptide in FIGS. 19A and 19B.

### Initial Engineering Based on 6.5.11 Register

The 6.5.11 register positions include that are predominantly beta-sheet, specifically amino acid positions 380, 382, 387, 422, 424, 426, 438, 440, according to EU numbering. An “NNK walk” library was generated that involved making one-by-one NNK mutations of residues at these positions. The library was built and expressed on the yeast surface. The library was sorted with one round of magnetic bead sorting to full-length human TfR1, circularly permuted human TfR apical domain, and circularly permuted cyno TfR apical domain. The library was then sorted with three rounds of FACS using human TfR ECD or human TfR apical domain, and a negative selection on neutravidin-650 secondary antibody for the final round. The resulting populations were tested for binding to human TfR1 in the presence or absence of excess holoTf or a competitor clone. The results show that TfR binding of the library population did not compete with holo-Tf, but did compete with clone 35.21. The resulting population was sequenced and the top 6 clones were shown to bind human TfR ECD and human TfR apical domain by yeast display. A single clone, 6.5.11.1 (see Table 21 for clone sequence), was selected to move forward because of its better affinity to TfR and its lack of potential sequence liabilities.

Clone 6.5.11.1 was expressed recombinantly and tested for human TfR apical domain binding by Biacore with an estimated affinity around 20-40 μM, with very weak cross-reactivity to cyno TfR apical domain (see Table 22). “Clone 6.5.11.1 bivalent” is a bivalent Fc-Fab fusion polypeptide comprising two Fc polypeptides each comprising the sequence of clone 6.5.11.1, fused to the anti-BACE1 Fab domain (1A11).

### Affinity Maturation Using NNK Patch Libraries Based on Clone 6.5.11.1

Additional libraries based on clone 6.5.11.1 were generated to improve binding affinity against human TfR1 and cyno TfR. Six patch libraries (6.5.11.5, 6.5.11.6, 6.5.11.7, 6.5.11.8, 6.5.11.9, and 6.5.11.10) were generated that have 4 or 5 positions in different surface patches randomized with the codon NNW (Table 21). In some of the libraries, the residue at position 384 (which was not part of the original register) was also randomized.

The six patch libraries were screened with one round of MACS sorting using human TfR apical domain. This round was sorted with human TfR apical domain premixed with streptavidin-650, followed by three rounds of FACS sorting using human or cyno TfR ECD. Libraries 6.5.11.5 and 6.5.11.7 returned clones with improved binding affinity to human and cyno TfR ECD. The top 12 clones (6.5.11.5.23, 6.5.11.5.42, 6.5.11.5.50, 6.5.11.5.58, 6.5.11.5.59, 6.5.11.5.60, 6.5.11.5.64, 6.5.11.5.66, 6.5.11.5.67, 6.5.11.5.74, 6.5.11.5.75, and 6.5.11.7.122) were sequenced and tested as single clones to human TfR ECD, cyno TfR ECD, circularly permuted human TfR apical domain, and circularly permuted cyno TfR apical domain.

Analysis of the top 12 clones showed that six beta-sheet positions at 382, 422, 424, 426, 438, and 440 were invariant in clones that showed improved binding affinity to TfR. Additionally, three positions at 380, 384, and 387 led to an increase in binding affinity compared to the original parent clone 6.5.11.1. Several clones with amino acid deletions between residues at positions 383 to 387 were found to have improved binding affinity to human TfR. The binding affinity of clones 6.5.11.5.42 and 6.5.11.5.50 was measured by Biacore, with the lead clone 6.5.11.5.42 having a binding affinity of 2 μM towards circularly permuted human TfR apical domain (Table 22). In this example, the term “bivalent” refers to an Fc dimer in which both Fc polypeptides contain a TfR-binding site. The term “monovalent” refers to an Fc dimer in which one of the two Fc polypeptides contains a TfR-binding site while the other Fc polypeptide does not contain a TfR-binding site. Each of the clones shown in Table 22 is fused to an anti-BACE1 Fab domain.

### PK/PD Evaluation of Clone 6.5.11.5.42 in Chimeric HuTfR^{apical} Knock-In Mice

Transgenic mice expressing human Tfrc apical domain within the murine Tfrc gene were generated using CRISPR/Cas9 technology (chimeric huTfR^{apical} knock-in mice). The resulting chimeric TfR was expressed in vivo under the control of the endogenous promoter. Chimeric huTfR^{apical} knock-in mice are described in International Patent Publication WO 2018/152285.

Fc fragments containing clone 6.5.11.5.42 fused to an anti-BACE1 Fab (2118) were dosed intravenously in chimeric huTfR^{apical} knock-in mice to measure the reduction of amyloid beta 40 (AP40) in the mouse brain. “Clone 6.5.11.5.42 biv:2H₈” is a bivalent Fc-Fab fusion polypeptide comprising two Fc polypeptides each comprising the sequence of clone 6.5.11.5.42, fused to the anti-BACE1 Fab domain (2118). “Clone 6.5.11.5.42 mono:2H8” is a monovalent Fc-Fab fusion polypeptide comprising a first Fc polypeptide containing the sequence of clone 6.5.11.5.42 and T366W knob mutation, and a second Fc polypeptide containing T366S, L368A, and Y407V hole mutations and not containing a TfR-binding site, fused to the anti-BACE1 Fab domain (2118). The term “bivalent” refers to an Fc dimer in which both Fc polypeptides contain a TfR-binding site. The term “monovalent” refers to an Fc dimer in which one of the two Fc polypeptides contains a TfR-binding site while the other Fc polypeptide does not contain a TfR-binding site. “Anti-BACE1 control” is a negative control without any TfR-binding site.

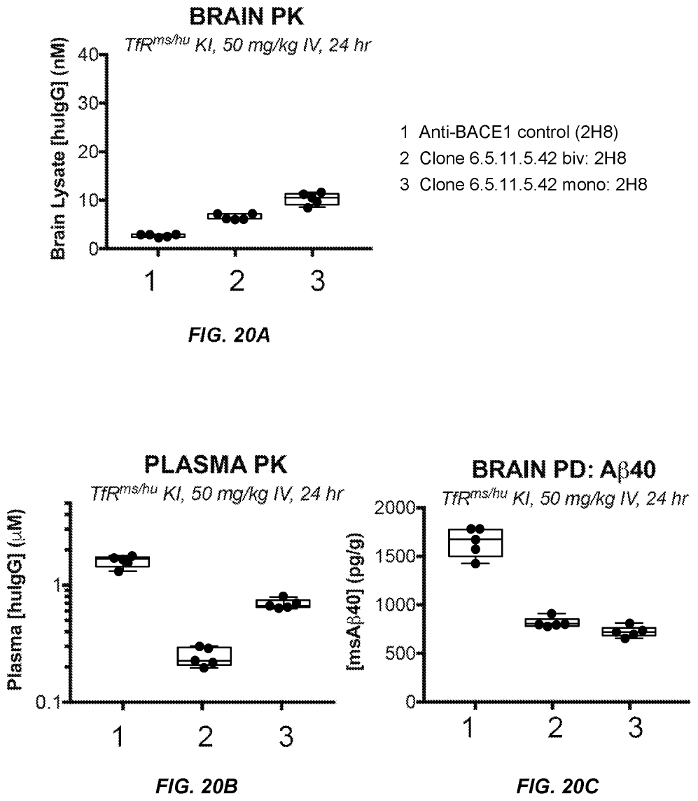

Clone 6.5.11.5.42 biv:2H₈, clone 6.5.11.5.42 mono:2H₈, and anti-BACE1 control were dosed at 50 mg/kg at 24 hours in chimeric huTfR^{apical} knock-in mice. Both clone 6.5.11.5.42 biv:2H₈ and clone 6.5.11.5.42 mono:2H₈ had reduction of brain Aβ40 and significant reduction compared to the negative control (FIGS. 20A-20C). Clone 6.5.11.5.42 biv:2H₈ and clone 6.5.11.5.42 mono:2H₈ also had a brain concentration higher than the negative control.

### Peripheral Walk Library of Clone 6.5.11.5.42

Clone 6.5.11.5.42 was subjected to additional engineering by yeast display for further affinity maturation to human and cyno TfR, as well as to improve the PK in wild-type mice. Additional mutations were added to the backbone (i.e., non-register) positions that were predicted to enhance binding through direct interactions, second-shell interaction, or structure stabilization. By looking at the structure of a wild-type human Fc (PDB No.: 4W4O), 12 peripheral residues hypothesized to increase the affinity to TfR were chosen. Peripheral residues mutated were at positions 378, 385, 386, 389, 390, 391, 421, 436, 437, 439, 441, and 442 (Table 21). These peripheral residues, as well as the original library residues, were mutated to NNK in separate libraries. The NNK walk involved making one-by-one NNK mutations of residues that are near the original register. Libraries were produced separately using degenerate primers and assembly of two PCR products as previously described, and expressed on the surface of yeast.

Each library population was independently analyzed by flow cytometry for the clones' binding to 50 nM human TfR apical domain and 50 nM cyno TfR ECD by yeast surface display. If improvement was seen relative to the parent, the top 5% of the library was sorted and sequenced. Additionally, libraries were pooled and sorted twice for circularly permuted human or cyno TfR apical domains for improved TfR binding compared to the parent. Residues that appeared in sorts showing improved binding are shown in Table 21. Interestingly, only positions 380, 384, and 387 in the original register were able to accept any residue changes without complete loss of target binding, while the residues at positions 382, 422, 424, 426, 438, and 440 are the same as those in the parent clone 6.5.11.5.42.

### Combinations of NNK Walk Libraries

The residues that most greatly improved affinity to TfR at positions 378, 380, 386. 387, 389, 390, and 391 from the peripheral walk (Table 21) were combined into two libraries and screened for improved TfR binding using yeast surface display. The first library, Hotspot Library 1, included E or Y at position 380, and NNK at positions 384, 385, 386, 387, and 389 (Table 21). The second library, Hotspot Library 2, included NNK at positions 378, 386, 389, 390, and 391, E or Y at position 380, and P or R at position 387. Each library was sorted once with human or cyno TfR apical domain by magnetic bead sorting, then sorted three times with human TfR ECD, cyno TfR ECD, or circularly permuted human TfR apical domain by FACS sorting. For the empty cells in Table 21, the amino acid at that position is the same as the one in the wild-type Fc.

The five best clones (clones 6.5.11.5.42.1, 6.5.11.5.42.2, 6.5.11.5.42.3, 6.5.11.5.42.4, and 6.5.11.5.42.8 in Table 21) were recombinantly expressed and tested by Biacore and cell binding. These five clones bound to human TfR with a binding affinity between 380 nM and 960 nM, and to cyno TfR with a binding affinity between 4.6 μM and less than 25 μM (Table 22).

TABLE 21

| | 378 | 379 | 380 | 381 | 382 | 383 | 384 | 385 | 386 | 387 | 388 | 389 | 390 | 391 | 392 | 393 |
|---|---|---|---|---|---|---|---|---|---|---|---|---|---|---|---|---|
| Wild-type | A | V | E | W | E | S | N | G | Q | P | E | N | N | Y | K | T |
| 6.5.11.1 | | | N | | F | | | | | I | | | | | | |
| 6.5.11.5 | | | * | | * | | * | | | * | | | | | | |
| 6.5.11.6 | | | N | | F | | | | | I | | | | | | |
| 6.5.11.7 | | | * | | F | | | | | * | | | | | | |
| 6.5.11.8 | | | N | | * | | * | | | I | | | | | | |
| 6.5.11.9 | | | * | | F | | | | | I | | | | | | |
| 6.5.11.10 | | | N | | * | | * | | | * | | | | | | |
| 6.5.11.5.23 | | | R | | F | | G | | | | | | | | | |
| 6.5.11.5.42 | | | Y | | F | | G | | | R | | | | | | |
| 6.5.11.5.50 | | | E | | F | | G | | | T | | | | | | |
| 6.5.11.5.58 | | | | | F | | F | | | K | | | | | | |
| 6.5.11.5.59 | | | E | | F | | F | | | T | | | | | | |
| 6.5.11.5.60 | | | I | | F | | X | X | X | X | | | | | | |
| 6.5.11.5.64 | | | | | F | | G | | | N | | | | | | |
| 6.5.11.5.66 | | | | | F | | F | | | R | | | | | | |
| 6.5.11.5.67 | | | S | | F | | D | | | S | | | | | | |
| 6.5.11.5.74 | | | F | | F | | E | | | R | | | | | | |
| 6.5.11.5.75 | | | Y | | F | | E | | | R | | | | | | |
| 6.5.11.7.122 | | | F | | F | | | | | R | | | | | | |
| Peripheral | * | | Y | | F | | G | * | * | R | | | * | * | | |
| walk based on | | | | | | | | | | | | | | | | |
| 6.5.11.5.42 | | | | | | | | | | | | | | | | |
| Residues that | E | | E | | | | F | | A | T | | Q | G | S | | |
| increased | L | | | | | | | | S | | | T | T | T | | |
| Wild-type | A | V | E | W | E | S | N | G | Q | P | E | N | N | Y | K | T |
| affinity to | I | | | | | | | | N | | | I | Y | I | | |
| human or | | | | | | | | | | | | S | | L | | |
| cyno TfR | | | | | | | | | | | | | | P | | |
| Hotspot | | | E/ | | F | | * | * | * | * | | * | | | | |
| Library 1 | | | Y | | | | | | | | | | | | | |
| Hotspot | * | | E/ | | F | | G | | * | P/ | | * | * | * | | |
| Library 2 | | | Y | | | | | | | R | | | | | | |
| 6.5.11.5.42.1 | | | | | F | | G | | A | K | | T | | | | |
| 6.5.11.5.42.2 | | | | | F | | D | | S | K | | T | | | | |
| 6.5.11.5.42.3 | | | | | F | | G | | S | K | | S | | | | |
| 6.5.11.5.42.4 | | | | | F | | G | | A | T | | S | | | | |
| 6.5.11.5.42.8 | | | | | F | | G | | A | R | L | P | L | | | |

| | 414 | 415 | 416 | 417 | 418 | 419 | 420 | 421 | 422 | 423 | 424 | 425 | 426 | 427 | 428 |
|---|---|---|---|---|---|---|---|---|---|---|---|---|---|---|---|
| Wild-type | K | S | R | W | Q | Q | G | N | V | F | S | C | S | V | M |
| 6.5.11.1 | | | | | | | | | L | | A | | E | | |
| 6.5.11.5 | | | | | | | | | L | | A | | E | | |
| 6.5.11.6 | | | | | | | | | * | | * | | * | | |
| 6.5.11.7 | | | | | | | | | * | | A | | E | | |
| 6.5.11.8 | | | | | | | | | * | | * | | * | | |
| 6.5.11.9 | | | | | | | | | L | | * | | * | | |
| 6.5.11.10 | | | | | | | | | * | | A | | E | | |
| 6.5.11.5.23 | | | | | | | | | L | | A | | E | | |
| 6.5.11.5.42 | | | | | | | | | L | | A | | E | | |
| 6.5.11.5.50 | | | | | | | | | L | | A | | E | | |
| 6.5.11.5.58 | | | | | | | | | L | | A | | E | | |
| 6.5.11.5.59 | | | | | | | | | L | | A | | E | | |
| 6.5.11.5.60 | | | | | | | | | L | | A | | E | | |
| 6.5.11.5.64 | | | | | | | | | L | | A | | E | | |
| 6.5.11.5.66 | | | | | | | | | L | | A | | E | | |
| 6.5.11.5.67 | | | | | | | | | L | | A | | E | | |
| 6.5.11.5.74 | | | | | | | | | L | | A | | E | | |
| 6.5.11.5.75 | | | | | | | | | L | | A | | E | | |
| 6.5.11.7.122 | | | | | | | | | L | | A | | E | | |
| Peripheral | | | | | | | | * | L | | A | | E | | |
| walk based on | | | | | | | | | | | | | | | |
| 6.5.11.5.42 | | | | | | | | | | | | | | | |
| Residues that | | | | | | | | A | | | | | | | |
| increased | | | | | | | | F | | | | | | | |
| Wild-type | K | S | R | W | Q | Q | G | N | V | F | S | C | S | V | M |
| affinity to | | | | | | | | G | | | | | | | |
| human or | | | | | | | | S | | | | | | | |
| cyno TfR | | | | | | | | Y | | | | | | | |
| Hotspot | | | | | | | | | L | | A | | E | | |
| Library 1 | | | | | | | | | | | | | | | |
| Hotspot | | | | | | | | | L | | A | | E | | |
| Library 2 | | | | | | | | | | | | | | | |
| 6.5.11.5.42.1 | | | | | | | | | L | | A | | E | | |
| 6.5.11.5.42.2 | | | | | | | | | L | | A | | E | | |
| 6.5.11.5.42.3 | | | | | | | | | L | | A | | E | | |
| 6.5.11.5.42.4 | | | | | | | | | L | | A | | E | | |
| 6.5.11.5.42.8 | P | | | K | | | | G | L | | A | | E | | |

| | 429 | 430 | 431 | 432 | 433 | 434 | 435 | 436 | 437 | 438 | 439 | 440 | 441 | 442 |
|---|---|---|---|---|---|---|---|---|---|---|---|---|---|---|
| Wild-type | H | E | A | L | H | N | H | Y | T | Q | K | S | L | S |
| 6.5.11.1 | | | | | | | | | | Y | | L | | |
| 6.5.11.5 | | | | | | | | | | Y | | L | | |
| 6.5.11.6 | | | | | | | | | | * | | * | | |
| 6.5.11.7 | | | | | | | | | | * | | * | | |
| 6.5.11.8 | | | | | | | | | | Y | | L | | |
| 6.5.11.9 | | | | | | | | | | * | | * | | |
| 6.5.11.10 | | | | | | | | | | Y | | L | | |
| 6.5.11.5.23 | | | | | | | | | | Y | | L | | |
| 6.5.11.5.42 | | | | | | | | | | Y | | L | | |
| 6.5.11.5.50 | | | | | | | | | | Y | | L | | |
| 6.5.11.5.58 | | | | | | | | | | Y | | L | | |
| 6.5.11.5.59 | | | | | | | | | | Y | | L | | |
| 6.5.11.5.60 | | | | | | | | | | Y | | L | | |
| 6.5.11.5.64 | | | | | | | | | | Y | | L | | |
| 6.5.11.5.66 | | | | | | | | | | Y | | L | | |
| 6.5.11.5.67 | | | | | | | | | | Y | | L | | |
| 6.5.11.5.74 | | | | | | | | | | Y | | L | | |
| 6.5.11.5.75 | | | | | | | | | | Y | | L | | |
| 6.5.11.7.122 | | | | | | | | | | Y | | L | | |
| Peripheral | | | | | | | | * | * | Y | * | L | * | * |
| walk based on | | | | | | | | | | | | | | |
| 6.5.11.5.42 | | | | | | | | | | | | | | |
| Residues that | | | | | | | | | D | | D | | | W |
| increased | | | | | | | | | | | E | | | |
| Wild-type | H | E | A | L | H | N | H | Y | T | Q | K | S | L | S |
| affinity to | | | | | | | | | | | S | | | |
| human or | | | | | | | | | | | | | | |
| cyno TfR | | | | | | | | | | | | | | |
| Hotspot | | | | | | | | | | Y | | L | | |
| Library 1 | | | | | | | | | | | | | | |
| Hotspot | | | | | | | | | | Y | | L | | |
| Library 2 | | | | | | | | | | | | | | |
| 6.5.11.5.42.1 | | | | | | | | | | Y | | L | | |
| 6.5.11.5.42.2 | | | | | | | | | | Y | | L | | |
| 6.5.11.5.42.3 | | | | | | | | | | Y | | L | | |
| 6.5.11.5.42.4 | | | | | | | | | | Y | | L | | |
| 6.5.11.5.42.8 | | | | | | | | | | Y | | L | | |

* indicates NNK walk.

TABLE 22

| | | | | K_{D} for | | K_{D} for | |
|---|---|---|---|---|---|---|---|
| | | | | huTfR | | cyTfR | |
| | | | | Apical | | Apical | |
| | Fc Dimer | First Fc Polypeptide | Second Fc Polypeptide | Domain | | Domain | |
| Initial | 6.5.11.1 | 6.5.11.1 TfR-binding site | 6.5.11.1 TfR-binding site | 20-40 | μM | Very | |
| clone | bivalent | | | | | weak | |
| Affinity | 6.5.11.5.42 | 6.5.11.5.42 TfR-binding site | 6.5.11.5.42 TfR-binding site | 2 | μM | Weak | |
| matured | bivalent | with LALA mutations | with LALA mutations | | | | |
| clones | 6.5.11.5.42 | 6.5.11.5.42 TfR-binding site | Fc sequence with T366S, | 2 | μM | Weak | |
| | mono | with T366W knob and | L368A, and Y407V hole | | | | |
| | | LALA mutations | and LALA mutations | | | | |
| | 6.5.11.5.50 | 6.5.11.5.50 TfR-binding site | Fc sequence with T366S, | 3 | μM | Weak | |
| | mono | with T366W knob and | L368A, and Y407V hole | | | | |
| | | LALA mutations | and LALA mutations | | | | |
| NNK | 6.5.11.5.42.1 | 6.5.11.5.42.1 TfR-binding | Fc sequence with T366S, | 840 | nM | 4.6 | μM |
| combos | mono | site with T366W knob and | L368A, and Y407V hole | | | | |
| | | LALA mutations | and LALA mutations | | | | |
| | 6.5.11.5.42.2 | 6.5.11.5.42.2 TfR-binding | Fc sequence with T366S, | 650 | nM | 7.5 | μM |
| | mono | site with T366W knob and | L368A, and Y407V hole | | | | |
| | | LALA mutations | and LALA mutations | | | | |
| | 6.5.11.5.42.3 | 6.5.11.5.42.3 TfR-binding | Fc sequence with T366S, | 920 | nM | 12.3 | μM |
| | mono | site with T366W knob and | L368A, and Y407V hole | | | | |
| | | LALA mutations | and LALA mutations | | | | |
| | 6.5.11.5.42.4 | 6.5.11.5.42.4 TfR-binding | Fc sequence with T366S, | 960 | nM | >25 | μM |
| | mono | site with T366W knob and | L368A, and Y407V hole | | | | |
| | | LALA mutations | and LALA mutations | | | | |
| | 6.5.11.5.42.8 | 6.5.11.5.42.4 TfR-binding | Fc sequence with T366S, | 380 | nM | 11.5 | μM |
| | mono | site with T366W knob and | L368A, and Y407V hole | | | | |
| | | LALA mutations | and LALA mutations | | | | |

### PK/PD Evaluation After Hotspot Affinity Maturation

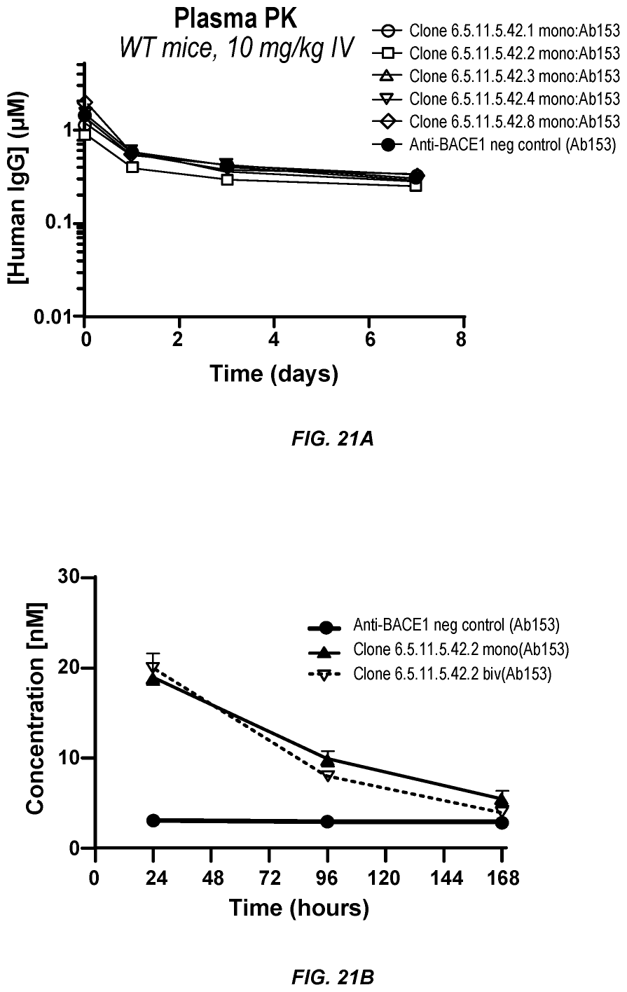

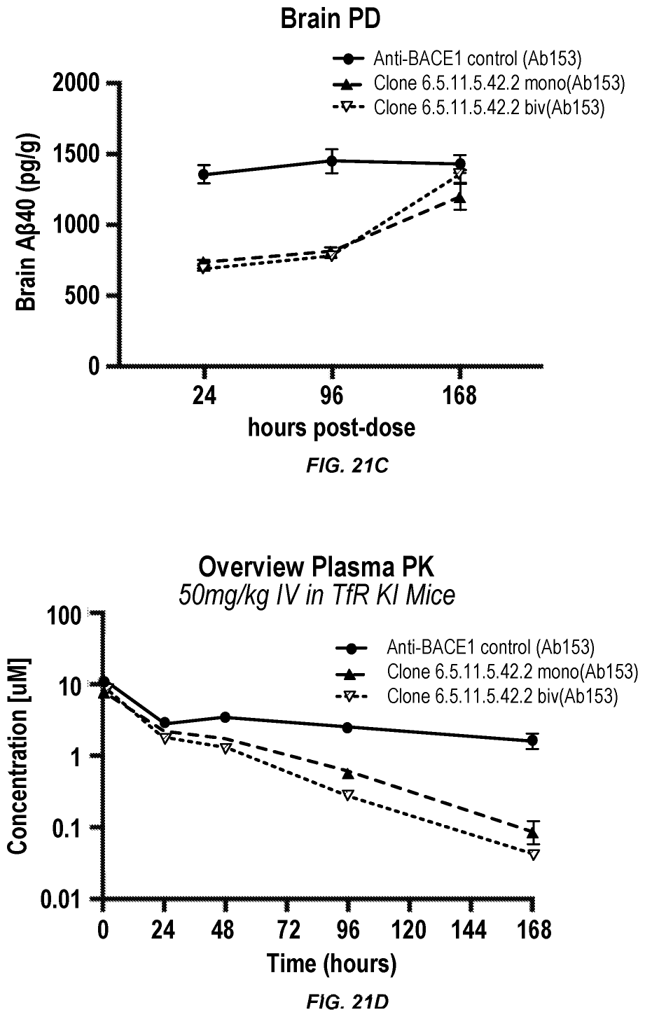

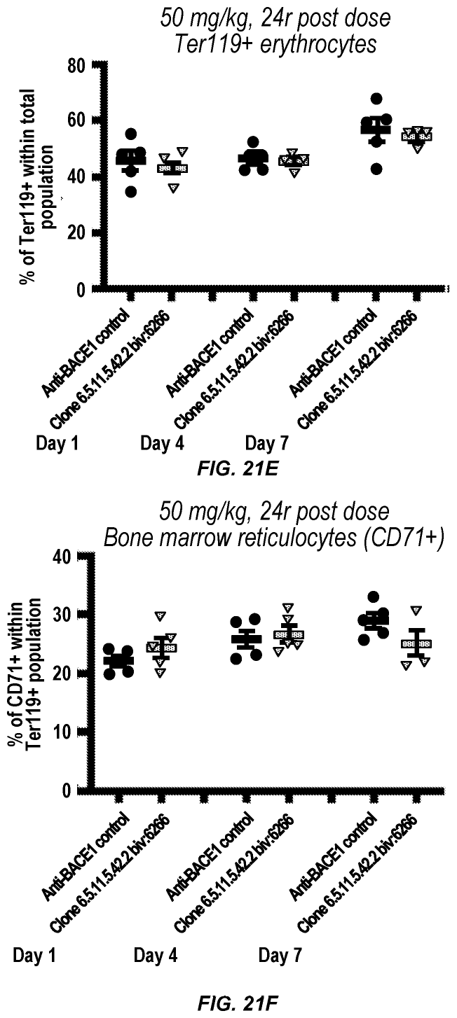

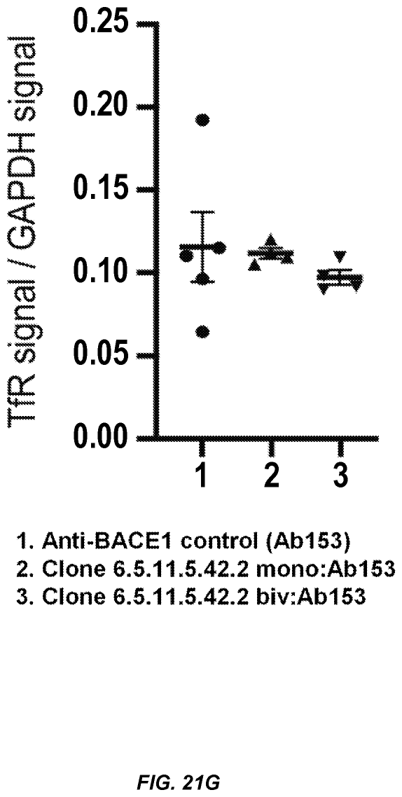

Clones 6.5.11.5.42.1, 6.5.11.5.42.2, 6.5.11.5.42.3, 6.5.11.5.42.4, and 6.5.11.5.42.8, containing the Fc polypeptides as listed in Table 23, were tested for their PK in wild-type TfR mice to ensure that there are no PK liabilities. In FIGS. 21A and 211B, all clones used were monovalent, except for “clone 6.5.11.5.42 biv:Ab153.” All the clones tested had normal clearance relative to a non-TfR binding control which had a clearance value of 10 mL/day/kg (FIG. 21A and Table 23).

TABLE 23

| Fc Dimer in | | | |
|---|---|---|---|
| Fc | | | |
| Dimer:Ab153 | | | Clearance |
| Fusion | First Fc Polypeptide | Second Fc Polypeptide | (mL/day/kg) |
| 6.5.11.5.42.1 | 6.5.11.5.42.1 TfR-binding site | Fc sequence with T366S, L368A, | 10 |
| mono | with T366W knob and LALA | and Y407V hole and LALA | |
| | mutations | mutations | |
| 6.5.11.5.42.2 | 6.5.11.5.42.2 TfR-binding site | Fc sequence with T366S, L368A, | 12 |
| mono | with T366W knob and LALA | and Y407V hole and LALA | |
| | mutations | mutations | |
| 6.5.11.5.42.3 | 6.5.11.5.42.3 TfR-binding site | Fc sequence with T366S, L368A, | 12 |
| mono | with T366W knob and LALA | and Y407V hole and LALA | |
| | mutations | mutations | |
| 6.5.11.5.42.4 | 6.5.11.5.42.4 TfR-binding site | Fc sequence with T366S, L368A, | 12 |
| mono | with T366W knob and LALA | and Y407V hole and LALA | |
| | mutations | mutations | |
| 6.5.11.5.42.8 | 6.5.11.5.42.8 TfR-binding site | Fc sequence with T366S, L368A, | 9 |
| mono | with T366W knob and LALA | and Y407V hole and LALA | |
| | mutations | mutations | |

In order to test rain uptake of the clones with improve PK, clone 6.5.11.5.42.2 was chosen based on its affinity for human TfR (K_{d}=650 nM). In FIGS. 21B-21G, “clone 6.5.11.5.42.2 biv:Ab153” is a bivalent Fc-Fab fusion polypeptide comprising two Fc polypeptides each comprising the sequence of clone 6.5.11.5.42.2 and LALA mutations, fused to the high affinity anti-BACE1 Fab domain (Ab153); and “Clone 6.5.11.5.42.2 mono:Ab153” is a monovalent Fc-Fab fusion polypeptide comprising a first Fc polypeptide containing the sequence of clone 6.5.11.5.42.2, T366W knob mutation, and LALA mutations, and a second Fc polypeptide containing T366S, L368A, and Y407V hole mutations, LALA mutations, and not containing a TfR-binding site, fused to the high affinity anti-BACE1 Fab domain (Ab153).

The clones and the controls were dosed intravenously into huTfR^{apical} knock-in mice at 50 mg/kg. Brain concentrations were measured in mouse brain at 24, 96, and 198 hours (FIG. 21B). Clone 6.5.11.5.42.2 mono:Ab153 and clone 6.5.11.5.42.2 biv:Ab153 showed good brain uptake with brain concentrations of 19.1 nM and 19.9 nM, respectively, at 24 hours post-dose compared to the negative control which showed brain concentration at 3.3 nM. These two clones also showed Aβ40 reduction in the brain at 24 hours post-dose (FIG. 21C). Brain concentration and brain Aβ40 concentration of these two clones had sustained brain concentrations and brain PD (Aβ40 reduction) out to 96 hours. Plasma PK showed a target-mediated clearance of all TfR-binding clones compared to the control as expected (FIG. 21D).

Previously, it had been shown that molecules that can bind two TfR molecules simultaneously can result in a decrease in circulating reticulocytes and TfR bone marrow cells. Given the crystal structure suggested only one TfR molecule could bind at a time, the levels of blood reticulocytes, Ter 19′ erythrocytes (FIG. 21E), and CD71⁺bone marrow reticulocytes (FIG. 21F) were measured. Changes in these values compared to the anti-BACE1 control were not observed. The effect of the clones on TfR levels was also measured (FIG. 21G). These results show that there is no difference in the levels of TfR, indicating that the clones did not cause TfR degradation.

### Dematuration/Reversion of Clone 6.5.11.5.42.2

In an effort to weaken the affinity of clone 6.5.11.5.42.2, several residues were reverted back to the wild-type residue or to a residue that had previously been identified to affect the affinity to the human TfR apical domain. These were done either as single point mutations or as combinations. Clones were expressed and purified from HEK293 cells as previously, and their affinity to the human TfR apical domain was measured by Biacore.

Table 24 shows the library of 6.5.11.5.42.2 mutants. Each mutant contained 1, 2, or 3 amino acid substitutions of 6.5.11.5.42.2. For example, one mutant may contain D384N and the amino acids at the rest of the positions are the same as those in 6.5.11.5.42.2. The positions shown in Table 24 are numbered according to the EU numbering scheme. The amino acid in each mutant that is different from that in 6.5.11.5.42.2 is in bold.

TABLE 24

| | | | | | | | | | | | | K_{D} for huTfR Apical |
|---|---|---|---|---|---|---|---|---|---|---|---|---|
| | 382 | 384 | 385 | 386 | 387 | 389 | 422 | 424 | 426 | 438 | 440 | Domain (nM) |
| 6.5.11.5.42.2 | F | D | G | S | K | T | L | A | E | Y | L | 650 |
| 6.5.11.5.42.2.d1 | F | N | G | S | K | T | L | A | E | Y | L | 3600 |
| 6.5.11.5.42.2.d2 | F | D | G | Q | K | T | L | A | E | Y | L | 3500 |
| 6.5.11.5.42.2.d3 | F | D | G | S | P | T | L | A | E | Y | L | 3500 |
| 6.5.11.5.42.2.d4 | F | D | G | G | K | T | L | A | E | Y | L | 8700 |
| 6.5.11.5.42.2.d5* | F | D | G | S | K | T | V | A | E | Y | L | NA |
| 6.5.11.5.42.2.d6 | F | F | G | S | K | T | L | A | E | Y | L | 24000 |
| 6.5.11.5.42.2.d7 | F | A | G | S | K | T | L | A | E | Y | L | 29000 |
| 6.5.11.5.42.2.d8 | F | D | A | S | K | T | L | A | E | Y | L | 4400 |
| 6.5.11.5.42.2.d9 | F | D | G | A | K | T | L | A | E | Y | L | 2000 |
| 6.5.11.5.42.2.d10 | F | D | G | S | T | T | L | A | E | Y | L | 1800 |
| 6.5.11.5.42.2.d11 | F | D | G | S | K | S | L | A | E | Y | L | 1800 |
| 6.5.11.5.42.2.d12 | F | D | G | S | P | N | L | A | E | Y | L | 4900 |
| 6.5.11.5.42.2.d13 | F | D | G | S | P | N | V | A | E | Y | L | No binding |
| 6.5.11.5.42.2.d14 | F | N | G | Q | K | T | L | A | E | Y | L | 9400 |
| 6.5.11.5.42.2.d15 | F | F | G | Q | K | T | L | A | E | Y | L | 2000 |

*Clone 6.5.11.5.42.2.d5 did not express.

### HIC Assessment of Library Clones

Clones 6.5.11.5.42.1, 6.5.11.5.42.2, 6.5.11.5.42.3, 6.5.11.5.42.4, and 6.5.11.5.42.8, containing the Fc polypeptides as listed in Table 23, were assessed by hydrophobic interaction (HIC) during developability assessment. Each clone is a monovalent Fc-Fab fusion polypeptide comprising (i) a first Fc polypeptide having the sequence of the identified clone, T366W knob mutation, and LALA mutations, (ii) a second Fc polypeptide containing T366S, L368A, and Y407V hole mutations, and LALA mutations, and (iii) the high affinity anti-BACE1 Fab domain (Ab153) linked to the Fc polypeptides. The control is the high affinity anti-BACE1 Fab domain (Ab153) linked to an Fc domain with LALA mutations and without TfR-binding sites.

Five (5) μg of each clone were injected onto two Thermo ProPac HIC-10 columns (5 μm, 4.6×100 mm, Cat. No. 63655) arranged in series. The mobile phases used in the columns are as follows: mobile phase A: 1× PBS, pH 7.4; mobile phase B: 1× PBS, 0.9 M Na₂SO₄; flow rate 0.75 mL/min, linear gradient starting at 80% mobile phase B for t=0 to 3 minutes, ramping down to 0% mobile phase B from t=3 to 19 minutes, holding at 0% mobile phase B for t=19 to 26 minutes, and returning to and holding at 80% mobile phase B from t=27 to 32 minutes, column temperature 25° C. Detection was carried out using fluorescence (excitation 290 nm/emission 325 nm), and the internal standard was NIST monoclonal antibody at 1 mg/mL. The results are illustrated in Table 25.

TABLE 25

| | HIC | | | Titer |
|---|---|---|---|---|
| Fc Dimer | RT (min) | Δ (min) | area % | (mg/L) |
| Wild type control | 17.6 | 0 | 90 | 612 |
| 6.5.11.5.42.1 mono | 23.5 | 5.9 | 86 | 787 |
| 6.5.11.5.42.2 mono | 21.9 | 4.3 | 89 | 808 |
| 6.5.11.5.42.3 mono | 22.9 | 5.3 | 90 | 801 |
| 6.5.11.5.42.4 mono | 23.5 | 5.9 | 93 | 833 |
| 6.5.11.5.42.8 mono | 22.6 | 5 | 86 | 896 |

All clones exhibited higher recombinant protein titer and more hydrophobic properties than the wild-type control. Clone 6.5.11.5.42.2 exhibited the least change in hydrophobicity with respect to the wild-type control (as illustrated by retention time, or RT) and was selected for further evaluation.

### Low pH Stability of Clone 6.5.11.5.42

Clone 6.5.11.5.42.2, with and without LS mutations, was assessed for low pH stability during developability assessment. The tested proteins are bivalent Fc-Fab fusion polypeptides comprising an anti-HER2 Fab domain linked to two Fc polypeptides each having the sequence of the identified clone and LALA mutations, with LS mutations or without LS mutations. An Fc control containing the anti-HER2 Fab domain linked to an Fc domain with LALA mutations and without TfR-binding sites was included for comparison.

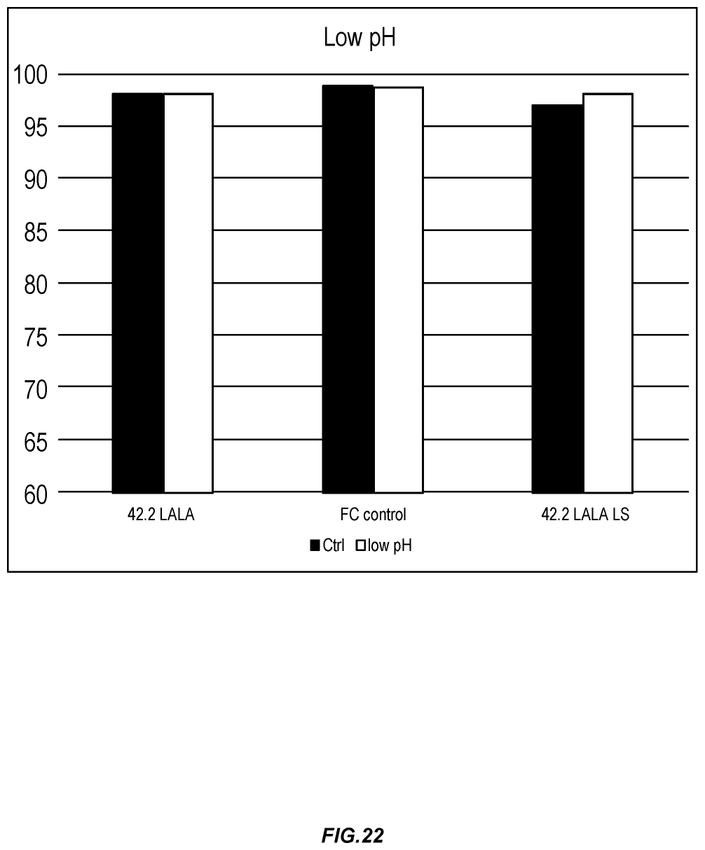

Aliquots (100 μL) of each clone were added to wells of a 96-well plate containing 6 μL of 5% (v/v) acetic acid and mixed well. The plate was sealed and incubated at room temperature for about three (3) hours. Subsequently, 35 μL of 1 M Tris HCl (pH 7.5) was added to the samples and mixed well. The plate was sealed once again and incubated at room temperature for 24 hours. Control condition was storage in 1× PBS without any change in pH during incubation. The samples were then analyzed by Biacore for binding affinity and by size exclusion chromatography to detect intact fusion protein. FIG. 22 and Table 26 provide the results of the analyses.

TABLE 26

| | TfR binding [K_{D} (μM)/% Rmax] | | |
|---|---|---|---|
| Sample | 42.2 LALA | Fc Control | 42.2 LALA LS |
| Control | 1.17/152.5 | NA | 1.70/140.8 |
| Low pH | 1.18/166.5 | NA | 1.81/156.6 |

As illustrated in FIG. 22 low pH exposure (followed by neutralization treatment) did not impact protein stability in clone 6.5.11.5.42.2. Using size-exclusion chromatography (SEC) analysis, minimal change was observed for the clone, which was true regardless of whether the LS mutations was present in the Fc polypeptides. Furthermore, low pH exposure and subsequent neutralization treatment also did not impact the TfR binding of the clone (Table 26). These results illustrate the stability of the clone despite being subjected to low pH stress conditions.

Additional Clones from Structure Library

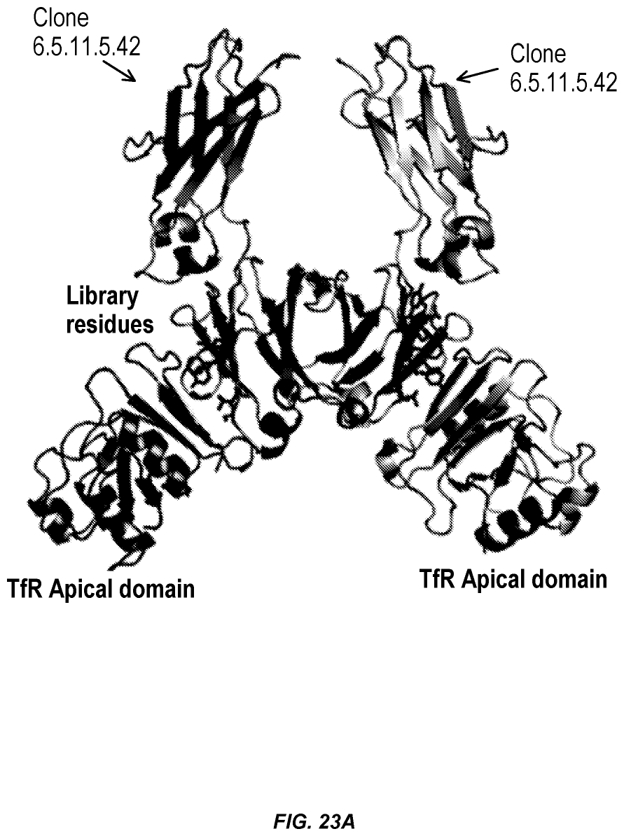

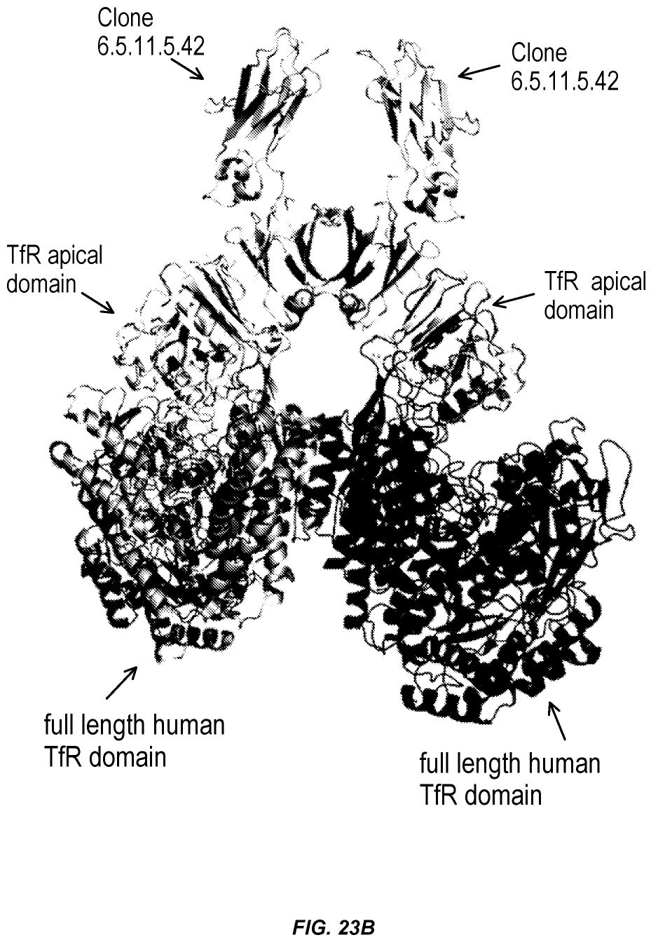

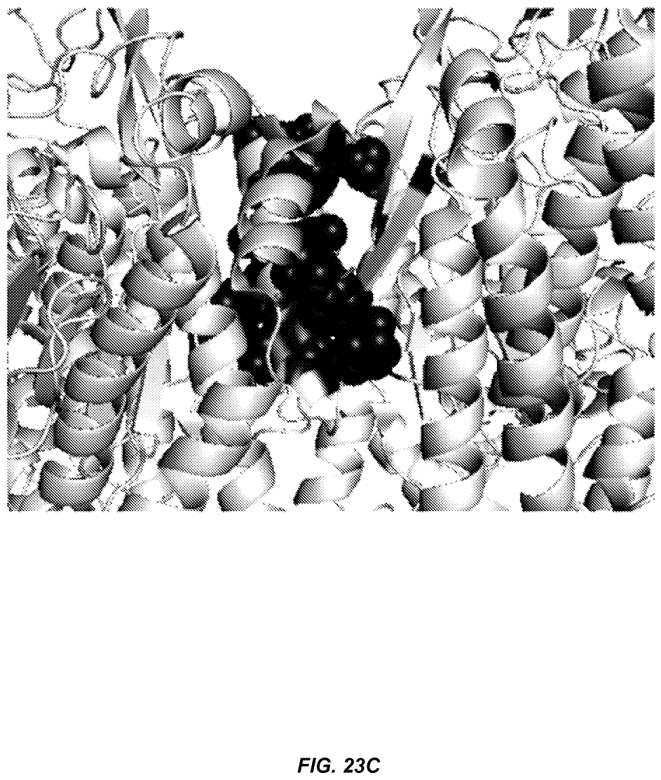

The structure of clone 6.5.11.5.42 with human TfR circularly permuted apical domain (FIGS. 23A-23C) was used to determine residue positions that were contacting or near the TfR apical domain. These positions were made into four separate libraries using clone 6.5.11.5.42.2 sequence with some residue positions mutated to the codon NNK or deletions. Libraries with 1 or 2 residue deletions were made in order to allow transferrin sidechains to have more room to bind.
(1) Libraryl: Positions 383, 385, 388, 389, and 391.
(2) Library 2: Positions 383, 384, deletion at 385, 386, 387, and 388.
(3) Library 3 is an equal mixture of three libraries: library 3a: positions 383, 384, deletion at 385, deletion at 386, 387, and 388; library 3b: positions 386, 387, deletion at 388, 390, and 391; and library 3c: 383, 384, 385, 386.
(4) Library 4: Positions 419-421, and 442-443.

Each mutant in Table 27 contained several amino acid substitutions relative to wild-type human IgG1 Fc. For the empty cells in Table 27, the amino acid at that position is the same as the one in the wild-type Fc. The clones were expressed recombinantly and tested for human TfR apical domain binding with an estimated affinity around 43-1000 nM, with very weak cross-reactivity to cyno TfR apical domain (Table 28).

TABLE 27

| | 382 | 383 | 384 | 385 | 386 | 387 | 388 | 389 | 419 | 420 | 421 | 422 |
|---|---|---|---|---|---|---|---|---|---|---|---|---|
| Wild-type | E | S | N | G | Q | P | E | N | Q | G | N | L |
| 42.2.1.2 | F | Y | D | D | S | K | L | T | | | | L |
| 42.2.1.20 | F | A | D | A | S | K | Q | R | | | | L |
| 42.2.2.10 | F | ~ | E | | N | G | D | T | | | | L |
| 42.2.3.1H | F | Y | G | N | A | K | | T | | | | L |
| 42.2.3.1C | F | G | T | N | K | K | | T | | | | L |
| 42.2.3.3 | F | T | D | N | Y | K | | T | | | | L |
| 42.2.3.4 | F | A | G | | G | K | | T | | | | L |
| 42.2.3.5 | F | Y | G | N | G | K | | T | | | | L |
| 42.2.5.2 | F | | D | | S | K | | T | P | R | G | L |
| 42.2.5.4 | F | | D | | S | K | | T | | Q | G | L |
| 42.2.7.2 | F | | D | | S | K | | T | | | | L |
| 42.2.19 | F | Y | D | D | S | K | | T | P | R | G | L |

| | 423 | 424 | 425 | 426 | 428 | 433 | 434 | 438 | 440 | 442 | 443 |
|---|---|---|---|---|---|---|---|---|---|---|---|
| Wild-type | F | S | C | S | M | H | N | Q | S | S | L |
| 42.2.1.2 | | A | | E | | | | Y | L | | |
| 42.2.1.20 | | A | | E | | | | Y | L | | |
| 42.2.2.10 | | A | | E | | | | Y | L | | |
| 42.2.3.1H | | A | | E | | | | Y | L | | |
| 42.2.3.1C | | A | | E | | | | Y | L | | |
| 42.2.3.3 | | A | | E | | | | Y | L | | |
| 42.2.3.4 | | A | | E | | | | Y | L | | |
| 42.2.3.5 | | A | | E | | | | Y | L | | |
| 42.2.5.2 | | A | | E | | | | Y | L | G | E |
| 42.2.5.4 | | A | | E | | | | Y | L | G | Y |
| 42.2.7.2 | | A | | E | E | E | G | Y | L | | |
| 42.2.19 | | A | | E | | | | Y | L | G | E |

~ indicates the amino acid is absent at position 383.

TABLE 28

| | | | K_{D} for | K_{D} for |
|---|---|---|---|---|
| | | | huTfR | cyTfR |
| | | | Apical | Apical |
| | | | Domain | Domain |
| Fc Dimer | First Fc Polypeptide | Second Fc Polypeptide | (nM) | (nM) |
| 42.2.1.2 | 42.2.1.2 TfR-binding site with | Fc sequence with T366S, | 284 | 7000 |
| mono | T366W knob, P329G, and | L368A, and Y407V hole, | | |
| | LALA mutations | P329G, and LALA mutations | | |
| 42.2.1.20 | 42.2.1.20 TfR-binding site | L368A, and Y407V hole, | 440 | 7000 |
| mono | with T366W knob, P329G, and | Fc sequence with T366S, | | |
| | LALA mutations | P329G, and LALA mutations | | |
| 42.2.2.10 | 42.2.2.10 TfR-binding site | L368A, and Y407V hole, | 43 | 7000 |
| mono | with T366W knob, P329G, and | Fc sequence with T366S, | | |
| | LALA mutations | P329G, and LALA mutations | | |
| 42.2.3.1H | 42.2.3.1H TfR-binding site | L368A, and Y407V hole, | 220 | 2000 |
| mono | with T366W knob, P329G, and | Fc sequence with T366S, | | |
| | LALA mutations | P329G, and LALA mutations | | |
| 42.2.3.1C | 42.2.3.1C TfR-binding site | L368A, and Y407V hole, | 1000 | 10,000 |
| mono | with T366W knob, P329G, and | Fc sequence with T366S, | | |
| | LALA mutations | P329G, and LALA mutations | | |
| 42.2.3.3 | 42.2.3.3 TfR-binding site with | Fc sequence with T366S, | 360 | 8000 |
| mono | T366W knob, P329G, and | L368A, and Y407V hole, | | |
| | LALA mutations | P329G, and LALA mutations | | |
| 42.2.3.4 | 42.2.3.4 TfR-binding site with | Fc sequence with T366S, | 181 | 4000 |
| mono | T366W knob, P329G, and | L368A, and Y407V hole, | | |
| | LALA mutations | P329G, and LALA mutations | | |
| 42.2.3.5 | 42.2.3.5 TfR-binding site with | Fc sequence with T366S, | 260 | 1800 |
| mono | T366W knob, P329G, and | L368A, and Y407V hole, | | |
| | LALA mutations | P329G, and LALA mutations | | |
| 42.2.5.2 | 42.2.5.2 TfR-binding site with | Fc sequence with T366S, | 180 | 2000 |
| mono | T366W knob, P329G, and | L368A, and Y407V hole, | | |
| | LALA mutations | P329G, and LALA mutations | | |
| 42.2.5.4 | 42.2.5.4 TfR-binding site with | Fc sequence with T366S, | 400 | 6500 |
| mono | T366W knob, P329G, and | L368A, and Y407V hole, | | |
| | LALA mutations | P329G, and LALA mutations | | |
| 42.2.7.2 | 42.2.7.2 TfR-binding site with | Fc sequence with T366S, | 162 | 10,000 |
| mono | T366W knob, P329G, and | L368A, and Y407V hole, | | |
| | LALA mutations | P329G, and LALA mutations | | |
| 42.2.19 | 42.2.19 TfR-binding site with | Fc sequence with T366S, | 79 | 1100 |
| mono | T366W knob, P329G, and | L368A, and Y407V hole, | | |
| | LALA mutations | P329G, and LALA mutations | | |

### PK/PD Evaluation of Clone 42.2.1.2

Clones 42.2.1.2 monovalent and 42.2.1.2 bivalent were tested for their PK in wild-type TfR mice to ensure that there are no PK liabilities. They were tested alongside previously tested clones 6.5.11.5.42.2 monovalent and 6.5.11.5.42.2 bivalent. Clone 42.2.1.2 monovalent used here contained 42.2.1.2 TfR-binding site with T366W knob, P329G, and LALA mutations as the first Fc polypeptide and Fc sequence with T366S, L368A, and Y407V hole, P329G, and LALA mutations as the second Fc polypeptide. Both of Fc polypeptides in clone 42.2.1.2 bivalent used here contained 42.2.1.2 TfR-binding site with P329G and LALA mutations. Clone 6.5.11.5.42.2 monovalent used here contained 6.5.11.5.42.2 TfR-binding site with T366W knob, P329G, and LALA mutations as the first Fc polypeptide and Fc sequence with T366S, L368A, and Y407V hole, P329G, and LALA mutations as the second Fc polypeptide. Both of Fc polypeptides in clone 6.5.11.5.42.2 bivalent used here contained 6.5.11.5.42.2 TfR-binding site with P329G and LALA mutations.

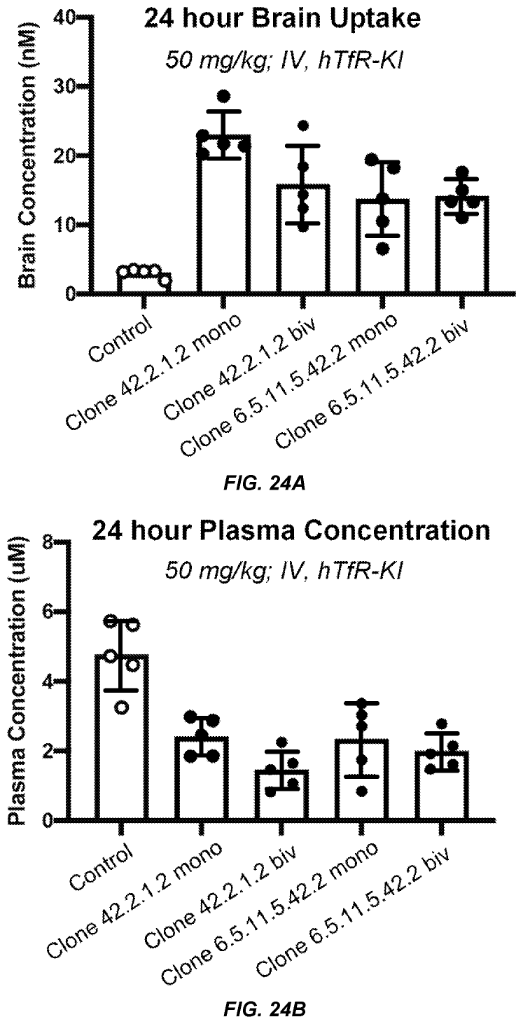

The clones and the controls were dosed intravenously into huTfR^{apical} knock-in mice at 50 mg/kg. Brain and plasma concentrations were measured at 24 hours (FIGS. 24A and 24B). Clone 42.2.1.2 mono and clone 42.2.1.2 biv showed good brain uptake at 24 hours post-dose compared to the negative control (FIG. 24A). Plasma PK showed a clearance of all TfR-binding clones compared to the control as expected (FIG. 24B).

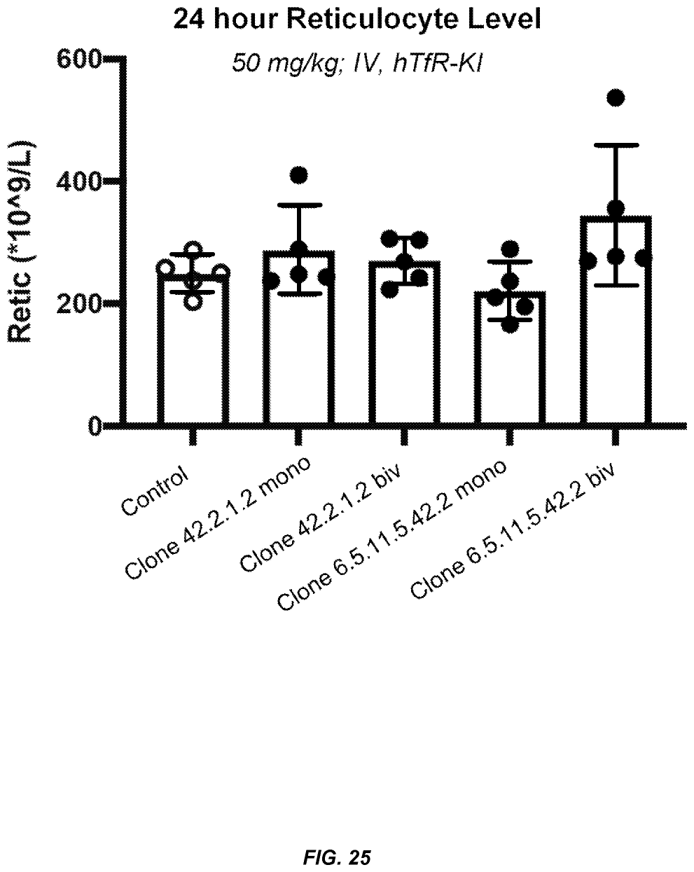

The effect of the clones on circulating reticulocytes was also investigated. FIG. 25 shows the measurement of Ter119^{+ erythrocytes. Changes in this value compared to the control was not observed.}

Further, concentrations of the clones in the brain and plasma were also measured in a time course experiment (see FIGS. 3B-3D). In this experiment, clone 42.2.1.2 mono:Ab153 contained 42.2.1.2 TfR-binding site with T366W knob and LALA mutations as the first Fc polypeptide, Fc sequence with T366S, L368A, and Y407V hole and LALA mutations as the second Fc polypeptide, and a high affinity anti-BACE1 Fab domain (Ab153) linked to the Fc polypeptides. Clone 42.2.1.2 biv:Ab153 contained two Fc polypeptides each having 42.2.1.2 TfR-binding site with LALA mutations and Ab153 linked to the Fc polypeptides. Anti-BACE1 control:Ab153 contained Ab153 linked to an Fc domain with P329G and LALA mutations and without TfR-binding sites. The clones showed Aβ40 reduction in the brain at 24 hours post-dose (see FIG. 3C). Brain concentration and brain Aβ40 concentration of the clones had sustained brain concentrations and brain PD (Aβ40 reduction) beyond 144 hours (see FIGS. 3B and 3C). Plasma PK showed a target-mediated clearance of all TfR-binding clones compared to the control as expected (see FIG. 3D).

Additional Clones from Rational Design

Based on the previous sorting results and sequences, the best residues at key positions in the clone that contributed to high affinity TfR binding were determined. To understand the relative impact of a few positions on binding and human/cyno cross-reactivity, a panel of rationally designed mutations at positions 382-389 were made. The clones contained 1, 2, 3, 4, 5, 6, 7, or 8 mutations at positions 382-389. 216 clones (Table 29) were made based on the sequence of clone 6.5.11.5.42.2 with the following substitutions in every combination: at position 383: S or Y; at position 384: G, D, or E; at position 385: D, A, or G; at position 386: Q or S; at position 388: E or L; and at position 389: N, T, or S. A few additional clones are also listed in Table 29. The positions listed in Table 29 are numbered according to EU numbering. For the positions not listed in Table 29, the amino acids at those positions are the same as those of the wild-type Fc polypeptide, except for clone 42.2.19, which has P at position 419, R at position 420, G at position 421, G at position 442, and E at position 443. In order to explore the affinity to TfR for every sequence combination at these positions, these clones were expressed in mammalian supernatant and their affinity to human and cyno apical domain was measured using Biacore™.

TABLE 29

| | 378 | 379 | 380 | 381 | 382 | 383 | 384 | 385 | 386 | 387 | 388 | 389 | 390 | 422 | 423 | 424 |
|---|---|---|---|---|---|---|---|---|---|---|---|---|---|---|---|---|
| Wild-type | A | V | E | W | E | S | N | G | Q | P | E | N | N | V | F | S |
| 42.8.1 | A | V | E | W | F | S | G | G | Q | K | E | N | N | L | F | A |
| 42.8.2 | A | V | E | W | F | S | G | G | Q | K | E | T | N | L | F | A |
| 42.8.3 | A | V | E | W | F | S | G | G | Q | K | E | S | N | L | F | A |
| 42.8.4 | A | V | E | W | F | S | G | G | Q | K | L | N | N | L | F | A |
| 42.8.5 | A | V | E | W | F | S | G | G | Q | K | L | T | N | L | F | A |
| 42.8.6 | A | V | E | W | F | S | G | G | Q | K | L | S | N | L | F | A |
| 42.8.7 | A | V | E | W | F | S | G | G | S | K | E | N | N | L | F | A |
| 42.8.8 | A | V | E | W | F | S | G | G | S | K | E | T | N | L | F | A |
| 42.8.9 | A | V | E | W | F | S | G | G | S | K | E | S | N | L | F | A |
| 42.8.10 | A | V | E | W | F | S | G | G | S | K | L | N | N | L | F | A |
| 42.8.11 | A | V | E | W | F | S | G | G | S | K | L | T | N | L | F | A |
| 42.8.12 | A | V | E | W | F | S | G | G | S | K | L | S | N | L | F | A |
| 42.8.13 | A | V | E | W | F | S | G | A | Q | K | E | N | N | L | F | A |
| 42.8.14 | A | V | E | W | F | S | G | A | Q | K | E | T | N | L | F | A |
| 42.8.15 | A | V | E | W | F | S | G | A | Q | K | E | S | N | L | F | A |
| 42.8.16 | A | V | E | W | F | S | G | A | Q | K | L | N | N | L | F | A |
| 42.8.17 | A | V | E | W | F | S | G | A | Q | K | L | T | N | L | F | A |
| 42.8.18 | A | V | E | W | F | S | G | A | Q | K | L | S | N | L | F | A |
| 42.8.19 | A | V | E | W | F | S | G | A | S | K | E | N | N | L | F | A |
| 42.8.20 | A | V | E | W | F | S | G | A | S | K | E | T | N | L | F | A |
| 42.8.21 | A | V | E | W | F | S | G | A | S | K | E | S | N | L | F | A |
| 42.8.22 | A | V | E | W | F | S | G | A | S | K | L | N | N | L | F | A |
| 42.8.23 | A | V | E | W | F | S | G | A | S | K | L | T | N | L | F | A |
| 42.8.24 | A | V | E | W | F | S | G | A | S | K | L | S | N | L | F | A |
| 42.8.25 | A | V | E | W | F | S | G | D | Q | K | E | N | N | L | F | A |
| 42.8.26 | A | V | E | W | F | S | G | D | Q | K | E | T | N | L | F | A |
| Wild-type | A | V | E | W | E | S | N | G | Q | P | E | N | N | V | F | S |
| 42.8.27 | A | V | E | W | F | S | G | D | Q | K | E | S | N | L | F | A |
| 42.8.28 | A | V | E | W | F | S | G | D | Q | K | L | N | N | L | F | A |
| 42.8.29 | A | V | E | W | F | S | G | D | Q | K | L | T | N | L | F | A |
| 42.8.30 | A | V | E | W | F | S | G | D | Q | K | L | S | N | L | F | A |
| 42.8.31 | A | V | E | W | F | S | G | D | S | K | E | N | N | L | F | A |
| 42.8.32 | A | V | E | W | F | S | G | D | S | K | E | T | N | L | F | A |
| 42.8.33 | A | V | E | W | F | S | G | D | S | K | E | S | N | L | F | A |
| 42.8.34 | A | V | E | W | F | S | G | D | S | K | L | N | N | L | F | A |
| 42.8.35 | A | V | E | W | F | S | G | D | S | K | L | T | N | L | F | A |
| 42.8.36 | A | V | E | W | F | S | G | D | S | K | L | S | N | L | F | A |
| 42.8.37 | A | V | E | W | F | S | D | G | Q | K | E | N | N | L | F | A |
| 42.8.38 | A | V | E | W | F | S | D | G | Q | K | E | T | N | L | F | A |
| 42.8.39 | A | V | E | W | F | S | D | G | Q | K | E | S | N | L | F | A |
| 42.8.40 | A | V | E | W | F | S | D | G | Q | K | L | N | N | L | F | A |
| 42.8.41 | A | V | E | W | F | S | D | G | Q | K | L | T | N | L | F | A |
| 42.8.42 | A | V | E | W | F | S | D | G | Q | K | L | S | N | L | F | A |
| 42.8.43 | A | V | E | W | F | S | D | G | S | K | E | N | N | L | F | A |
| 6.5.11.5.42.2 | A | V | E | W | F | S | D | G | S | K | E | T | N | L | F | A |
| 42.8.45 | A | V | E | W | F | S | D | G | S | K | E | S | N | L | F | A |
| 42.8.46 | A | V | E | W | F | S | D | G | S | K | L | N | N | L | F | A |
| 42.8.47 | A | V | E | W | F | S | D | G | S | K | L | T | N | L | F | A |
| 42.8.48 | A | V | E | W | F | S | D | G | S | K | L | S | N | L | F | A |
| 42.8.49 | A | V | E | W | F | S | D | A | Q | K | E | N | N | L | F | A |
| 42.8.50 | A | V | E | W | F | S | D | A | Q | K | E | T | N | L | F | A |
| 42.8.51 | A | V | E | W | F | S | D | A | Q | K | E | S | N | L | F | A |
| 42.8.52 | A | V | E | W | F | S | D | A | Q | K | L | N | N | L | F | A |
| 42.8.53 | A | V | E | W | F | S | D | A | Q | K | L | T | N | L | F | A |
| 42.8.54 | A | V | E | W | F | S | D | A | Q | K | L | S | N | L | F | A |
| Wild-type | A | V | E | W | E | S | N | G | Q | P | E | N | N | V | F | S |
| 42.8.55 | A | V | E | W | F | S | D | A | S | K | E | N | N | L | F | A |
| 42.8.56 | A | V | E | W | F | S | D | A | S | K | E | T | N | L | F | A |
| 42.8.57 | A | V | E | W | F | S | D | A | S | K | E | S | N | L | F | A |
| 42.8.58 | A | V | E | W | F | S | D | A | S | K | L | N | N | L | F | A |
| 42.8.59 | A | V | E | W | F | S | D | A | S | K | L | T | N | L | F | A |
| 42.8.60 | A | V | E | W | F | S | D | A | S | K | L | S | N | L | F | A |
| 42.8.61 | A | V | E | W | F | S | D | D | Q | K | E | N | N | L | F | A |
| 42.8.62 | A | V | E | W | F | S | D | D | Q | K | E | T | N | L | F | A |
| 42.8.63 | A | V | E | W | F | S | D | D | Q | K | E | S | N | L | F | A |
| 42.8.64 | A | V | E | W | F | S | D | D | Q | K | L | N | N | L | F | A |
| 42.8.65 | A | V | E | W | F | S | D | D | Q | K | L | T | N | L | F | A |
| 42.8.66 | A | V | E | W | F | S | D | D | Q | K | L | S | N | L | F | A |
| 42.8.67 | A | V | E | W | F | S | D | D | S | K | E | N | N | L | F | A |
| 42.8.68 | A | V | E | W | F | S | D | D | S | K | E | T | N | L | F | A |
| 42.8.69 | A | V | E | W | F | S | D | D | S | K | E | S | N | L | F | A |
| 42.8.70 | A | V | E | W | F | S | D | D | S | K | L | N | N | L | F | A |
| 42.8.71 | A | V | E | W | F | S | D | D | S | K | L | T | N | L | F | A |
| 42.8.72 | A | V | E | W | F | S | D | D | S | K | L | S | N | L | F | A |
| 42.8.73 | A | V | E | W | F | S | E | G | Q | K | E | N | N | L | F | A |
| 42.8.74 | A | V | E | W | F | S | E | G | Q | K | E | T | N | L | F | A |
| 42.8.75 | A | V | E | W | F | S | E | G | Q | K | E | S | N | L | F | A |
| 42.8.76 | A | V | E | W | F | S | E | G | Q | K | L | N | N | L | F | A |
| 42.8.77 | A | V | E | W | F | S | E | G | Q | K | L | T | N | L | F | A |
| 42.8.78 | A | V | E | W | F | S | E | G | Q | K | L | S | N | L | F | A |
| 42.8.79 | A | V | E | W | F | S | E | G | S | K | E | N | N | L | F | A |
| 42.8.80 | A | V | E | W | F | S | E | G | S | K | E | T | N | L | F | A |
| 42.8.81 | A | V | E | W | F | S | E | G | S | K | E | S | N | L | F | A |
| 42.8.82 | A | V | E | W | F | S | E | G | S | K | L | N | N | L | F | A |
| Wild-type | A | V | E | W | E | S | N | G | Q | P | E | N | N | V | F | S |
| 42.8.83 | A | V | E | W | F | S | E | G | S | K | L | T | N | L | F | A |
| 42.8.84 | A | V | E | W | F | S | E | G | S | K | L | S | N | L | F | A |
| 42.8.85 | A | V | E | W | F | S | E | A | Q | K | E | N | N | L | F | A |
| 42.8.86 | A | V | E | W | F | S | E | A | Q | K | E | T | N | L | F | A |
| 42.8.87 | A | V | E | W | F | S | E | A | Q | K | E | S | N | L | F | A |
| 42.8.88 | A | V | E | W | F | S | E | A | Q | K | L | N | N | L | F | A |
| 42.8.89 | A | V | E | W | F | S | E | A | Q | K | L | T | N | L | F | A |
| 42.8.90 | A | V | E | W | F | S | E | A | Q | K | L | S | N | L | F | A |
| 42.8.91 | A | V | E | W | F | S | E | A | S | K | E | N | N | L | F | A |
| 42.8.92 | A | V | E | W | F | S | E | A | S | K | E | T | N | L | F | A |
| 42.8.93 | A | V | E | W | F | S | E | A | S | K | E | S | N | L | F | A |
| 42.8.94 | A | V | E | W | F | S | E | A | S | K | L | N | N | L | F | A |
| 42.8.95 | A | V | E | W | F | S | E | A | S | K | L | T | N | L | F | A |
| 42.8.96 | A | V | E | W | F | S | E | A | S | K | L | S | N | L | F | A |
| 42.8.97 | A | V | E | W | F | S | E | D | Q | K | E | N | N | L | F | A |
| 42.8.98 | A | V | E | W | F | S | E | D | Q | K | E | T | N | L | F | A |
| 42.8.99 | A | V | E | W | F | S | E | D | O | K | E | S | N | L | F | A |
| 42.8.100 | A | V | E | W | F | S | E | D | Q | K | L | N | N | L | F | A |
| 42.8.101 | A | V | E | W | F | S | E | D | Q | K | L | T | N | L | F | A |
| 42.8.102 | A | V | E | W | F | S | E | D | Q | K | L | S | N | L | F | A |
| 42.8.103 | A | V | E | W | F | S | E | D | S | K | E | N | N | L | F | A |
| 42.8.104 | A | V | E | W | F | S | E | D | S | K | E | T | N | L | F | A |
| 42.8.105 | A | V | E | W | F | S | E | D | S | K | E | S | N | L | F | A |
| 42.8.106 | A | V | E | W | F | S | E | D | S | K | L | N | N | L | F | A |
| 42.8.107 | A | V | E | W | F | S | E | D | S | K | L | T | N | L | F | A |
| 42.8.108 | A | V | E | W | F | S | E | D | S | K | L | S | N | L | F | A |
| 42.8.109 | A | V | E | W | F | Y | G | G | Q | K | E | N | N | L | F | A |
| 42.8.110 | A | V | E | W | F | Y | G | G | Q | K | E | T | N | L | F | A |
| Wild-type | A | V | E | W | E | S | N | G | Q | P | E | N | N | V | F | S |
| 42.8.111 | A | V | E | W | F | Y | G | G | Q | K | E | S | N | L | F | A |
| 42.8.112 | A | V | E | W | F | Y | G | G | Q | K | L | N | N | L | F | A |
| 42.8.113 | A | V | E | W | F | Y | G | G | Q | K | L | T | N | L | F | A |
| 42.8.114 | A | V | E | W | F | Y | G | G | Q | K | L | S | N | L | F | A |
| 42.8.115 | A | V | E | W | F | Y | G | G | S | K | E | N | N | L | F | A |
| 42.8.116 | A | V | E | W | F | Y | G | G | S | K | E | T | N | L | F | A |
| 42.8.117 | A | V | E | W | F | Y | G | G | S | K | E | S | N | L | F | A |
| 42.8.118 | A | V | E | W | F | Y | G | G | S | K | L | N | N | L | F | A |
| 42.8.119 | A | V | E | W | F | Y | G | G | S | K | L | T | N | L | F | A |
| 42.8.120 | A | V | E | W | F | Y | G | G | S | K | L | S | N | L | F | A |
| 42.8.121 | A | V | E | W | F | Y | G | A | Q | K | E | N | N | L | F | A |
| 42.8.122 | A | V | E | W | F | Y | G | A | Q | K | E | T | N | L | F | A |
| 42.8.123 | A | V | E | W | F | Y | G | A | Q | K | E | S | N | L | F | A |
| 42.8.124 | A | V | E | W | F | Y | G | A | Q | K | L | N | N | L | F | A |
| 42.8.125 | A | V | E | W | F | Y | G | A | Q | K | L | T | N | L | F | A |
| 42.8.126 | A | V | E | W | F | Y | G | A | Q | K | L | S | N | L | F | A |
| 42.8.127 | A | V | E | W | F | Y | G | A | S | K | E | N | N | L | F | A |
| 42.8.128 | A | V | E | W | F | Y | G | A | S | K | E | T | N | L | F | A |
| 42.8.129 | A | V | E | W | F | Y | G | A | S | K | E | S | N | L | F | A |
| 42.8.130 | A | V | E | W | F | Y | G | A | S | K | L | N | N | L | F | A |
| 42.8.131 | A | V | E | W | F | Y | G | A | S | K | L | T | N | L | F | A |
| 42.8.132 | A | V | E | W | F | Y | G | A | S | K | L | S | N | L | F | A |
| 42.8.133 | A | V | E | W | F | Y | G | D | Q | K | E | N | N | L | F | A |
| 42.8.134 | A | V | E | W | F | Y | G | D | Q | K | E | T | N | L | F | A |
| 42.8.135 | A | V | E | W | F | Y | G | D | Q | K | E | S | N | L | F | A |
| 42.8.136 | A | V | E | W | F | Y | G | D | Q | K | L | N | N | L | F | A |
| 42.8.137 | A | V | E | W | F | Y | G | D | Q | K | L | T | N | L | F | A |
| 42.8.138 | A | V | E | W | F | Y | G | D | Q | K | L | S | N | L | F | A |
| Wild-type | A | V | E | W | E | S | N | G | Q | P | E | N | N | V | F | S |
| 42.8.139 | A | V | E | W | F | Y | G | D | S | K | E | N | N | L | F | A |
| 42.8.140 | A | V | E | W | F | Y | G | D | S | K | E | T | N | L | F | A |
| 42.8.141 | A | V | E | W | F | Y | G | D | S | K | E | S | N | L | F | A |
| 42.8.142 | A | V | E | W | F | Y | G | D | S | K | L | N | N | L | F | A |
| 42.8.143 | A | V | E | W | F | Y | G | D | S | K | L | T | N | L | F | A |
| 42.8.144 | A | V | E | W | F | Y | G | D | S | K | L | S | N | L | F | A |
| 42.8.145 | A | V | E | W | F | Y | D | G | Q | K | E | N | N | L | F | A |
| 42.8.146 | A | V | E | W | F | Y | D | G | Q | K | E | T | N | L | F | A |
| 42.8.147 | A | V | E | W | F | Y | D | G | Q | K | E | S | N | L | F | A |
| 42.8.148 | A | V | E | W | F | Y | D | G | Q | K | L | N | N | L | F | A |
| 42.8.149 | A | V | E | W | F | Y | D | G | Q | K | L | T | N | L | F | A |
| 42.8.150 | A | V | E | W | F | Y | D | G | Q | K | L | S | N | L | F | A |
| 42.8.151 | A | V | E | W | F | Y | D | G | S | K | E | N | N | L | F | A |
| 42.8.152 | A | V | E | W | F | Y | D | G | S | K | E | T | N | L | F | A |
| 42.8.153 | A | V | E | W | F | Y | D | G | S | K | E | S | N | L | F | A |
| 42.8.154 | A | V | E | W | F | Y | D | G | S | K | L | N | N | L | F | A |
| 42.8.155 | A | V | E | W | F | Y | D | G | S | K | L | T | N | L | F | A |
| 42.8.156 | A | V | E | W | F | Y | D | G | S | K | L | S | N | L | F | A |
| 42.8.157 | A | V | E | W | F | Y | D | A | Q | K | E | N | N | L | F | A |
| 42.8.158 | A | V | E | W | F | Y | D | A | Q | K | E | T | N | L | F | A |
| 42.8.159 | A | V | E | W | F | Y | D | A | Q | K | E | S | N | L | F | A |
| 42.8.160 | A | V | E | W | F | Y | D | A | Q | K | L | N | N | L | F | A |
| 42.8.161 | A | V | E | W | F | Y | D | A | Q | K | L | T | N | L | F | A |
| 42.8.162 | A | V | E | W | F | Y | D | A | Q | K | L | S | N | L | F | A |
| 42.8.163 | A | V | E | W | F | Y | D | A | S | K | E | N | N | L | F | A |
| 42.8.164 | A | V | E | W | F | Y | D | A | S | K | E | T | N | L | F | A |
| 42.8.165 | A | V | E | W | F | Y | D | A | S | K | E | S | N | L | F | A |
| 42.8.166 | A | V | E | W | F | Y | D | A | S | K | L | N | N | L | F | A |
| Wild-type | A | V | E | W | E | S | N | G | Q | P | E | N | N | V | F | S |
| 42.8.167 | A | V | E | W | F | Y | D | A | S | K | L | T | N | L | F | A |
| 42.8.168 | A | V | E | W | F | Y | D | A | S | K | L | S | N | L | F | A |
| 42.8.169 | A | V | E | W | F | Y | D | D | Q | K | E | N | N | L | F | A |
| 42.8.170 | A | V | E | W | F | Y | D | D | Q | K | E | T | N | L | F | A |
| 42.8.171 | A | V | E | W | F | Y | D | D | Q | K | E | S | N | L | F | A |
| 42.8.172 | A | V | E | W | F | Y | D | D | Q | K | L | N | N | L | F | A |
| 42.8.173 | A | V | E | W | F | Y | D | D | Q | K | L | T | N | L | F | A |
| 42.8.174 | A | V | E | W | F | Y | D | D | O | K | L | S | N | L | F | A |
| 42.8.175 | A | V | E | W | F | Y | D | D | S | K | E | N | N | L | F | A |
| 42.8.176 | A | V | E | W | F | Y | D | D | S | K | E | T | N | L | F | A |
| 42.8.177 | A | V | E | W | F | Y | D | D | S | K | E | S | N | L | F | A |
| 42.8.178 | A | V | E | W | F | Y | D | D | S | K | L | N | N | L | F | A |
| 42.2.1.2 | A | V | E | W | F | Y | D | D | S | K | L | T | N | L | F | A |
| 42.8.180 | A | V | E | W | F | Y | D | D | S | K | L | S | N | L | F | A |
| 42.8.181 | A | V | E | W | F | Y | E | G | Q | K | E | N | N | L | F | A |
| 42.8.182 | A | V | E | W | F | Y | E | G | Q | K | E | T | N | L | F | A |
| 42.8.183 | A | V | E | W | F | Y | E | G | Q | K | E | S | N | L | F | A |
| 42.8.184 | A | V | E | W | F | Y | E | G | Q | K | L | N | N | L | F | A |
| 42.8.185 | A | V | E | W | F | Y | E | G | Q | K | L | T | N | L | F | A |
| 42.8.186 | A | V | E | W | F | Y | E | G | Q | K | L | S | N | L | F | A |
| 42.8.187 | A | V | E | W | F | Y | E | G | S | K | E | N | N | L | F | A |
| 42.8.188 | A | V | E | W | F | Y | E | G | S | K | E | T | N | L | F | A |
| 42.8.189 | A | V | E | W | F | Y | E | G | S | K | E | S | N | L | F | A |
| 42.8.190 | A | V | E | W | F | Y | E | G | S | K | L | N | N | L | F | A |
| 42.8.191 | A | V | E | W | F | Y | E | G | S | K | L | T | N | L | F | A |
| 42.8.192 | A | V | E | W | F | Y | E | G | S | K | L | S | N | L | F | A |
| 42.8.193 | A | V | E | W | F | Y | E | A | Q | K | E | N | N | L | F | A |
| 42.8.194 | A | V | E | W | F | Y | E | A | Q | K | E | T | N | L | F | A |
| Wild-type | A | V | E | W | E | S | N | G | Q | P | E | N | N | V | F | S |
| 42.8.195 | A | V | E | W | F | Y | E | A | Q | K | E | S | N | L | F | A |
| 42.8.196 | A | V | E | W | F | Y | E | A | Q | K | L | N | N | L | F | A |
| 42.8.197 | A | V | E | W | F | Y | E | A | Q | K | L | T | N | L | F | A |
| 42.8.198 | A | V | E | W | F | Y | E | A | Q | K | L | S | N | L | F | A |
| 42.8.199 | A | V | E | W | F | Y | E | A | S | K | E | N | N | L | F | A |
| 42.8.200 | A | V | E | W | F | Y | E | A | S | K | E | T | N | L | F | A |
| 42.8.201 | A | V | E | W | F | Y | E | A | S | K | E | S | N | L | F | A |
| 42.8.202 | A | V | E | W | F | Y | E | A | S | K | L | N | N | L | F | A |
| 42.8.203 | A | V | E | W | F | Y | E | A | S | K | L | T | N | L | F | A |
| 42.8.204 | A | V | E | W | F | Y | E | A | S | K | L | S | N | L | F | A |
| 42.8.205 | A | V | E | W | F | Y | E | D | Q | K | E | N | N | L | F | A |
| 42.8.206 | A | V | E | W | F | Y | E | D | Q | K | E | T | N | L | F | A |
| 42.8.207 | A | V | E | W | F | Y | E | D | Q | K | E | S | N | L | F | A |
| 42.8.208 | A | V | E | W | F | Y | E | D | Q | K | L | N | N | L | F | A |
| 42.8.209 | A | V | E | W | F | Y | E | D | Q | K | L | T | N | L | F | A |
| 42.8.210 | A | V | E | W | F | Y | E | D | Q | K | L | S | N | L | F | A |
| 42.8.211 | A | V | E | W | F | Y | E | D | S | K | E | N | N | L | F | A |
| 42.8.212 | A | V | E | W | F | Y | E | D | S | K | E | T | N | L | F | A |
| 42.8.213 | A | V | E | W | F | Y | E | D | S | K | E | S | N | L | F | A |
| 42.8.214 | A | V | E | W | F | Y | E | D | S | K | L | N | N | L | F | A |
| 42.8.215 | A | V | E | W | F | Y | E | D | S | K | L | T | N | L | F | A |
| 42.8.216 | A | V | E | W | F | Y | E | D | S | K | L | S | N | L | F | A |
| 6.5.11.1 | A | V | N | W | F | S | N | G | Q | I | E | N | N | L | F | A |
| 6.5.11.7.122 | A | V | F | W | F | S | N | G | Q | R | E | N | N | L | F | A |
| 42.2.19 | A | V | E | W | F | Y | D | D | S | K | L | T | N | L | F | A |
| 42.2.7-2 | A | V | E | W | F | S | D | G | S | K | E | T | N | L | F | A |
| 42.2.3-5 | A | V | E | W | F | Y | G | N | G | K | E | T | N | L | F | A |
| 42.2.3-1H | A | V | E | W | F | Y | G | N | A | K | E | T | N | L | F | A |
| Wild-type | A | V | E | W | E | S | N | G | Q | P | E | N | N | V | F | S |
| 42.2.2-10 | A | V | E | W | F | * | E | G | N | G | D | T | N | L | F | A |
| 42.2.1-20 | A | V | E | W | F | A | D | A | S | K | Q | R | N | L | F | A |
| 6.5.11.5.42 | A | V | Y | W | F | S | G | G | Q | R | E | N | N | L | F | A |
| | 425 | 426 | 427 | 428 | 429 | 430 | 431 | 432 | 433 | 434 | 435 | 436 | 437 | 438 | 439 | 440 |
| Wild-type | C | S | V | M | H | E | A | L | H | N | H | Y | T | Q | K | S |
| 42.8.1 | C | E | V | M | H | E | A | L | H | N | H | Y | T | Y | K | L |
| 42.8.2 | C | E | V | M | H | E | A | L | H | N | H | Y | T | Y | K | L |
| 42.8.3 | C | E | V | M | H | E | A | L | H | N | H | Y | T | Y | K | L |
| 42.8.4 | C | E | V | M | H | E | A | L | H | N | H | Y | T | Y | K | L |
| 42.8.5 | C | E | V | M | H | E | A | L | H | N | H | Y | T | Y | K | L |
| 42.8.6 | C | E | V | M | H | E | A | L | H | N | H | Y | T | Y | K | L |
| 42.8.7 | C | E | V | M | H | E | A | L | H | N | H | Y | T | Y | K | L |
| 42.8.8 | C | E | V | M | H | E | A | L | H | N | H | Y | T | Y | K | L |
| 42.8.9 | C | E | V | M | H | E | A | L | H | N | H | Y | T | Y | K | L |
| 42.8.10 | C | E | V | M | H | E | A | L | H | N | H | Y | T | Y | K | L |
| 42.8.11 | C | E | V | M | H | E | A | L | H | N | H | Y | T | Y | K | L |
| 42.8.12 | C | E | V | M | H | E | A | L | H | N | H | Y | T | Y | K | L |
| 42.8.13 | C | E | V | M | H | E | A | L | H | N | H | Y | T | Y | K | L |
| 42.8.14 | C | E | V | M | H | E | A | L | H | N | H | Y | T | Y | K | L |
| 42.8.15 | C | E | V | M | H | E | A | L | H | N | H | Y | T | Y | K | L |
| 42.8.16 | C | E | V | M | H | E | A | L | H | N | H | Y | T | Y | K | L |
| 42.8.17 | C | E | V | M | H | E | A | L | H | N | H | Y | T | Y | K | L |
| 42.8.18 | C | E | V | M | H | E | A | L | H | N | H | Y | T | Y | K | L |
| 42.8.19 | C | E | V | M | H | E | A | L | H | N | H | Y | T | Y | K | L |
| 42.8.20 | C | E | V | M | H | E | A | L | H | N | H | Y | T | Y | K | L |
| 42.8.21 | C | E | V | M | H | E | A | L | H | N | H | Y | T | Y | K | L |
| 42.8.22 | C | E | V | M | H | E | A | L | H | N | H | Y | T | Y | K | L |
| 42.8.23 | C | E | V | M | H | E | A | L | H | N | H | Y | T | Y | K | L |
| 42.8.24 | C | E | V | M | H | E | A | L | H | N | H | Y | T | Y | K | L |
| 42.8.25 | C | E | V | M | H | E | A | L | H | N | H | Y | T | Y | K | L |
| 42.8.26 | C | E | V | M | H | E | A | L | H | N | H | Y | T | Y | K | L |
| Wild-type | C | S | V | M | H | E | A | L | H | N | H | Y | T | Q | K | S |
| 42.8.27 | C | E | V | M | H | E | A | L | H | N | H | Y | T | Y | K | L |
| 42.8.28 | C | E | V | M | H | E | A | L | H | N | H | Y | T | Y | K | L |
| 42.8.29 | C | E | V | M | H | E | A | L | H | N | H | Y | T | Y | K | L |
| 42.8.30 | C | E | V | M | H | E | A | L | H | N | H | Y | T | Y | K | L |
| 42.8.31 | C | E | V | M | H | E | A | L | H | N | H | Y | T | Y | K | L |
| 42.8.32 | C | E | V | M | H | E | A | L | H | N | H | Y | T | Y | K | L |
| 42.8.33 | C | E | V | M | H | E | A | L | H | N | H | Y | T | Y | K | L |
| 42.8.34 | C | E | V | M | H | E | A | L | H | N | H | Y | T | Y | K | L |
| 42.8.35 | C | E | V | M | H | E | A | L | H | N | H | Y | T | Y | K | L |
| 42.8.36 | C | E | V | M | H | E | A | L | H | N | H | Y | T | Y | K | L |
| 42.8.37 | C | E | V | M | H | E | A | L | H | N | H | Y | T | Y | K | L |
| 42.8.38 | C | E | V | M | H | E | A | L | H | N | H | Y | T | Y | K | L |
| 42.8.39 | C | E | V | M | H | E | A | L | H | N | H | Y | T | Y | K | L |
| 42.8.40 | C | E | V | M | H | E | A | L | H | N | H | Y | T | Y | K | L |
| 42.8.41 | C | E | V | M | H | E | A | L | H | N | H | Y | T | Y | K | L |
| 42.8.42 | C | E | V | M | H | E | A | L | H | N | H | Y | T | Y | K | L |
| 42.8.43 | C | E | V | M | H | E | A | L | H | N | H | Y | T | Y | K | L |
| 6.5.11.5.42.2 | C | E | V | M | H | E | A | L | H | N | H | Y | T | Y | K | L |
| 42.8.45 | C | E | V | M | H | E | A | L | H | N | H | Y | T | Y | K | L |
| 42.8.46 | C | E | V | M | H | E | A | L | H | N | H | Y | T | Y | K | L |
| 42.8.47 | C | E | V | M | H | E | A | L | H | N | H | Y | T | Y | K | L |
| 42.8.48 | C | E | V | M | H | E | A | L | H | N | H | Y | T | Y | K | L |
| 42.8.49 | C | E | V | M | H | E | A | L | H | N | H | Y | T | Y | K | L |
| 42.8.50 | C | E | V | M | H | E | A | L | H | N | H | Y | T | Y | K | L |
| 42.8.51 | C | E | V | M | H | E | A | L | H | N | H | Y | T | Y | K | L |
| 42.8.52 | C | E | V | M | H | E | A | L | H | N | H | Y | T | Y | K | L |
| 42.8.53 | C | E | V | M | H | E | A | L | H | N | H | Y | T | Y | K | L |
| 42.8.54 | C | E | V | M | H | E | A | L | H | N | H | Y | T | Y | K | L |
| Wild-type | C | S | V | M | H | E | A | L | H | N | H | Y | T | Q | K | S |
| 42.8.55 | C | E | V | M | H | E | A | L | H | N | H | Y | T | Y | K | L |
| 42.8.56 | C | E | V | M | H | E | A | L | H | N | H | Y | T | Y | K | L |
| 42.8.57 | C | E | V | M | H | E | A | L | H | N | H | Y | T | Y | K | L |
| 42.8.58 | C | E | V | M | H | E | A | L | H | N | H | Y | T | Y | K | L |
| 42.8.59 | C | E | V | M | H | E | A | L | H | N | H | Y | T | Y | K | L |
| 42.8.60 | C | E | V | M | H | E | A | L | H | N | H | Y | T | Y | K | L |
| 42.8.61 | C | E | V | M | H | E | A | L | H | N | H | Y | T | Y | K | L |
| 42.8.62 | C | E | V | M | H | E | A | L | H | N | H | Y | T | Y | K | L |
| 42.8.63 | C | E | V | M | H | E | A | L | H | N | H | Y | T | Y | K | L |
| 42.8.64 | C | E | V | M | H | E | A | L | H | N | H | Y | T | Y | K | L |
| 42.8.65 | C | E | V | M | H | E | A | L | H | N | H | Y | T | Y | K | L |
| 42.8.66 | C | E | V | M | H | E | A | L | H | N | H | Y | T | Y | K | L |
| 42.8.67 | C | E | V | M | H | E | A | L | H | N | H | Y | T | Y | K | L |
| 42.8.68 | C | E | V | M | H | E | A | L | H | N | H | Y | T | Y | K | L |
| 42.8.69 | C | E | V | M | H | E | A | L | H | N | H | Y | T | Y | K | L |
| 42.8.70 | C | E | V | M | H | E | A | L | H | N | H | Y | T | Y | K | L |
| 42.8.71 | C | E | V | M | H | E | A | L | H | N | H | Y | T | Y | K | L |
| 42.8.72 | C | E | V | M | H | E | A | L | H | N | H | Y | T | Y | K | L |
| 42.8.73 | C | E | V | M | H | E | A | L | H | N | H | Y | T | Y | K | L |
| 42.8.74 | C | E | V | M | H | E | A | L | H | N | H | Y | T | Y | K | L |
| 42.8.75 | C | E | V | M | H | E | A | L | H | N | H | Y | T | Y | K | L |
| 42.8.76 | C | E | V | M | H | E | A | L | H | N | H | Y | T | Y | K | L |
| 42.8.77 | C | E | V | M | H | E | A | L | H | N | H | Y | T | Y | K | L |
| 42.8.78 | C | E | V | M | H | E | A | L | H | N | H | Y | T | Y | K | L |
| 42.8.79 | C | E | V | M | H | E | A | L | H | N | H | Y | T | Y | K | L |
| 42.8.80 | C | E | V | M | H | E | A | L | H | N | H | Y | T | Y | K | L |
| 42.8.81 | C | E | V | M | H | E | A | L | H | N | H | Y | T | Y | K | L |
| 42.8.82 | C | E | V | M | H | E | A | L | H | N | H | Y | T | Y | K | L |
| Wild-type | C | S | V | M | H | E | A | L | H | N | H | Y | T | Q | K | S |
| 42.8.83 | C | E | V | M | H | E | A | L | H | N | H | Y | T | Y | K | L |
| 42.8.84 | C | E | V | M | H | E | A | L | H | N | H | Y | T | Y | K | L |
| 42.8.85 | C | E | V | M | H | E | A | L | H | N | H | Y | T | Y | K | L |
| 42.8.86 | C | E | V | M | H | E | A | L | H | N | H | Y | T | Y | K | L |
| 42.8.87 | C | E | V | M | H | E | A | L | H | N | H | Y | T | Y | K | L |
| 42.8.88 | C | E | V | M | H | E | A | L | H | N | H | Y | T | Y | K | L |
| 42.8.89 | C | E | V | M | H | E | A | L | H | N | H | Y | T | Y | K | L |
| 42.8.90 | C | E | V | M | H | E | A | L | H | N | H | Y | T | Y | K | L |
| 42.8.91 | C | E | V | M | H | E | A | L | H | N | H | Y | T | Y | K | L |
| 42.8.92 | C | E | V | M | H | E | A | L | H | N | H | Y | T | Y | K | L |
| 42.8.93 | C | E | V | M | H | E | A | L | H | N | H | Y | T | Y | K | L |
| 42.8.94 | C | E | V | M | H | E | A | L | H | N | H | Y | T | Y | K | L |
| 42.8.95 | C | E | V | M | H | E | A | L | H | N | H | Y | T | Y | K | L |
| 42.8.96 | C | E | V | M | H | E | A | L | H | N | H | Y | T | Y | K | L |
| 42.8.97 | C | E | V | M | H | E | A | L | H | N | H | Y | T | Y | K | L |
| 42.8.98 | C | E | V | M | H | E | A | L | H | N | H | Y | T | Y | K | L |
| 42.8.99 | C | E | V | M | H | E | A | L | H | N | H | Y | T | Y | K | L |
| 42.8.100 | C | E | V | M | H | E | A | L | H | N | H | Y | T | Y | K | L |
| 42.8.101 | C | E | V | M | H | E | A | L | H | N | H | Y | T | Y | K | L |
| 42.8.102 | C | E | V | M | H | E | A | L | H | N | H | Y | T | Y | K | L |
| 42.8.103 | C | E | V | M | H | E | A | L | H | N | H | Y | T | Y | K | L |
| 42.8.104 | C | E | V | M | H | E | A | L | H | N | H | Y | T | Y | K | L |
| 42.8.105 | C | E | V | M | H | E | A | L | H | N | H | Y | T | Y | K | L |
| 42.8.106 | C | E | V | M | H | E | A | L | H | N | H | Y | T | Y | K | L |
| 42.8.107 | C | E | V | M | H | E | A | L | H | N | H | Y | T | Y | K | L |
| 42.8.108 | C | E | V | M | H | E | A | L | H | N | H | Y | T | Y | K | L |
| 42.8.109 | C | E | V | M | H | E | A | L | H | N | H | Y | T | Y | K | L |
| 42.8.110 | C | E | V | M | H | E | A | L | H | N | H | Y | T | Y | K | L |
| Wild-type | C | S | V | M | H | E | A | L | H | N | H | Y | T | Q | K | S |
| 42.8.111 | C | E | V | M | H | E | A | L | H | N | H | Y | T | Y | K | L |
| 42.8.112 | C | E | V | M | H | E | A | L | H | N | H | Y | T | Y | K | L |
| 42.8.113 | C | E | V | M | H | E | A | L | H | N | H | Y | T | Y | K | L |
| 42.8.114 | C | E | V | M | H | E | A | L | H | N | H | Y | T | Y | K | L |
| 42.8.115 | C | E | V | M | H | E | A | L | H | N | H | Y | T | Y | K | L |
| 42.8.116 | C | E | V | M | H | E | A | L | H | N | H | Y | T | Y | K | L |
| 42.8.117 | C | E | V | M | H | E | A | L | H | N | H | Y | T | Y | K | L |
| 42.8.118 | C | E | V | M | H | E | A | L | H | N | H | Y | T | Y | K | L |
| 42.8.119 | C | E | V | M | H | E | A | L | H | N | H | Y | T | Y | K | L |
| 42.8.120 | C | E | V | M | H | E | A | L | H | N | H | Y | T | Y | K | L |
| 42.8.121 | C | E | V | M | H | E | A | L | H | N | H | Y | T | Y | K | L |
| 42.8.122 | C | E | V | M | H | E | A | L | H | N | H | Y | T | Y | K | L |
| 42.8.123 | C | E | V | M | H | E | A | L | H | N | H | Y | T | Y | K | L |
| 42.8.124 | C | E | V | M | H | E | A | L | H | N | H | Y | T | Y | K | L |
| 42.8.125 | C | E | V | M | H | E | A | L | H | N | H | Y | T | Y | K | L |
| 42.8.126 | C | E | V | M | H | E | A | L | H | N | H | Y | T | Y | K | L |
| 42.8.127 | C | E | V | M | H | E | A | L | H | N | H | Y | T | Y | K | L |
| 42.8.128 | C | E | V | M | H | E | A | L | H | N | H | Y | T | Y | K | L |
| 42.8.129 | C | E | V | M | H | E | A | L | H | N | H | Y | T | Y | K | L |
| 42.8.130 | C | E | V | M | H | E | A | L | H | N | H | Y | T | Y | K | L |
| 42.8.131 | C | E | V | M | H | E | A | L | H | N | H | Y | T | Y | K | L |
| 42.8.132 | C | E | V | M | H | E | A | L | H | N | H | Y | T | Y | K | L |
| 42.8.133 | C | E | V | M | H | E | A | L | H | N | H | Y | T | Y | K | L |
| 42.8.134 | C | E | V | M | H | E | A | L | H | N | H | Y | T | Y | K | L |
| 42.8.135 | C | E | V | M | H | E | A | L | H | N | H | Y | T | Y | K | L |
| 42.8.136 | C | E | V | M | H | E | A | L | H | N | H | Y | T | Y | K | L |
| 42.8.137 | C | E | V | M | H | E | A | L | H | N | H | Y | T | Y | K | L |
| 42.8.138 | C | E | V | M | H | E | A | L | H | N | H | Y | T | Y | K | L |
| Wild-type | C | S | V | M | H | E | A | L | H | N | H | Y | T | Q | K | S |
| 42.8.139 | C | E | V | M | H | E | A | L | H | N | H | Y | T | Y | K | L |
| 42.8.140 | C | E | V | M | H | E | A | L | H | N | H | Y | T | Y | K | L |
| 42.8.141 | C | E | V | M | H | E | A | L | H | N | H | Y | T | Y | K | L |
| 42.8.142 | C | E | V | M | H | E | A | L | H | N | H | Y | T | Y | K | L |
| 42.8.143 | C | E | V | M | H | E | A | L | H | N | H | Y | T | Y | K | L |
| 42.8.144 | C | E | V | M | H | E | A | L | H | N | H | Y | T | Y | K | L |
| 42.8.145 | C | E | V | M | H | E | A | L | H | N | H | Y | T | Y | K | L |
| 42.8.146 | C | E | V | M | H | E | A | L | H | N | H | Y | T | Y | K | L |
| 42.8.147 | C | E | V | M | H | E | A | L | H | N | H | Y | T | Y | K | L |
| 42.8.148 | C | E | V | M | H | E | A | L | H | N | H | Y | T | Y | K | L |
| 42.8.149 | C | E | V | M | H | E | A | L | H | N | H | Y | T | Y | K | L |
| 42.8.150 | C | E | V | M | H | E | A | L | H | N | H | Y | T | Y | K | L |
| 42.8.151 | C | E | V | M | H | E | A | L | H | N | H | Y | T | Y | K | L |
| 42.8.152 | C | E | V | M | H | E | A | L | H | N | H | Y | T | Y | K | L |
| 42.8.153 | C | E | V | M | H | E | A | L | H | N | H | Y | T | Y | K | L |
| 42.8.154 | C | E | V | M | H | E | A | L | H | N | H | Y | T | Y | K | L |
| 42.8.155 | C | E | V | M | H | E | A | L | H | N | H | Y | T | Y | K | L |
| 42.8.156 | C | E | V | M | H | E | A | L | H | N | H | Y | T | Y | K | L |
| 42.8.157 | C | E | V | M | H | E | A | L | H | N | H | Y | T | Y | K | L |
| 42.8.158 | C | E | V | M | H | E | A | L | H | N | H | Y | T | Y | K | L |
| 42.8.159 | C | E | V | M | H | E | A | L | H | N | H | Y | T | Y | K | L |
| 42.8.160 | C | E | V | M | H | E | A | L | H | N | H | Y | T | Y | K | L |
| 42.8.161 | C | E | V | M | H | E | A | L | H | N | H | Y | T | Y | K | L |
| 42.8.162 | C | E | V | M | H | E | A | L | H | N | H | Y | T | Y | K | L |
| 42.8.163 | C | E | V | M | H | E | A | L | H | N | H | Y | T | Y | K | L |
| 42.8.164 | C | E | V | M | H | E | A | L | H | N | H | Y | T | Y | K | L |
| 42.8.165 | C | E | V | M | H | E | A | L | H | N | H | Y | T | Y | K | L |
| 42.8.166 | C | E | V | M | H | E | A | L | H | N | H | Y | T | Y | K | L |
| Wild-type | C | S | V | M | H | E | A | L | H | N | H | Y | T | Q | K | S |
| 42.8.167 | C | E | V | M | H | E | A | L | H | N | H | Y | T | Y | K | L |
| 42.8.168 | C | E | V | M | H | E | A | L | H | N | H | Y | T | Y | K | L |
| 42.8.169 | C | E | V | M | H | E | A | L | H | N | H | Y | T | Y | K | L |
| 42.8.170 | C | E | V | M | H | E | A | L | H | N | H | Y | T | Y | K | L |
| 42.8.171 | C | E | V | M | H | E | A | L | H | N | H | Y | T | Y | K | L |
| 42.8.172 | C | E | V | M | H | E | A | L | H | N | H | Y | T | Y | K | L |
| 42.8.173 | C | E | V | M | H | E | A | L | H | N | H | Y | T | Y | K | L |
| 42.8.174 | C | E | V | M | H | E | A | L | H | N | H | Y | T | Y | K | L |
| 42.8.175 | C | E | V | M | H | E | A | L | H | N | H | Y | T | Y | K | L |
| 42.8.176 | C | E | V | M | H | E | A | L | H | N | H | Y | T | Y | K | L |
| 42.8.177 | C | E | V | M | H | E | A | L | H | N | H | Y | T | Y | K | L |
| 42.8.178 | C | E | V | M | H | E | A | L | H | N | H | Y | T | Y | K | L |
| 42.2.1.2 | C | E | V | M | H | E | A | L | H | N | H | Y | T | Y | K | L |
| 42.8.180 | C | E | V | M | H | E | A | L | H | N | H | Y | T | Y | K | L |
| 42.8.181 | C | E | V | M | H | E | A | L | H | N | H | Y | T | Y | K | L |
| 42.8.182 | C | E | V | M | H | E | A | L | H | N | H | Y | T | Y | K | L |
| 42.8.183 | C | E | V | M | H | E | A | L | H | N | H | Y | T | Y | K | L |
| 42.8.184 | C | E | V | M | H | E | A | L | H | N | H | Y | T | Y | K | L |
| 42.8.185 | C | E | V | M | H | E | A | L | H | N | H | Y | T | Y | K | L |
| 42.8.186 | C | E | V | M | H | E | A | L | H | N | H | Y | T | Y | K | L |
| 42.8.187 | C | E | V | M | H | E | A | L | H | N | H | Y | T | Y | K | L |
| 42.8.188 | C | E | V | M | H | E | A | L | H | N | H | Y | T | Y | K | L |
| 42.8.189 | C | E | V | M | H | E | A | L | H | N | H | Y | T | Y | K | L |
| 42.8.190 | C | E | V | M | H | E | A | L | H | N | H | Y | T | Y | K | L |
| 42.8.191 | C | E | V | M | H | E | A | L | H | N | H | Y | T | Y | K | L |
| 42.8.192 | C | E | V | M | H | E | A | L | H | N | H | Y | T | Y | K | L |
| 42.8.193 | C | E | V | M | H | E | A | L | H | N | H | Y | T | Y | K | L |
| 42.8.194 | C | E | V | M | H | E | A | L | H | N | H | Y | T | Y | K | L |
| Wild-type | C | S | V | M | H | E | A | L | H | N | H | Y | T | Q | K | S |
| 42.8.195 | C | E | V | M | H | E | A | L | H | N | H | Y | T | Y | K | L |
| 42.8.196 | C | E | V | M | H | E | A | L | H | N | H | Y | T | Y | K | L |
| 42.8.197 | C | E | V | M | H | E | A | L | H | N | H | Y | T | Y | K | L |
| 42.8.198 | C | E | V | M | H | E | A | L | H | N | H | Y | T | Y | K | L |
| 42.8.199 | C | E | V | M | H | E | A | L | H | N | H | Y | T | Y | K | L |
| 42.8.200 | C | E | V | M | H | E | A | L | H | N | H | Y | T | Y | K | L |
| 42.8.201 | C | E | V | M | H | E | A | L | H | N | H | Y | T | Y | K | L |
| 42.8.202 | C | E | V | M | H | E | A | L | H | N | H | Y | T | Y | K | L |
| 42.8.203 | C | E | V | M | H | E | A | L | H | N | H | Y | T | Y | K | L |
| 42.8.204 | C | E | V | M | H | E | A | L | H | N | H | Y | T | Y | K | L |
| 42.8.205 | C | E | V | M | H | E | A | L | H | N | H | Y | T | Y | K | L |
| 42.8.206 | C | E | V | M | H | E | A | L | H | N | H | Y | T | Y | K | L |
| 42.8.207 | C | E | V | M | H | E | A | L | H | N | H | Y | T | Y | K | L |
| 42.8.208 | C | E | V | M | H | E | A | L | H | N | H | Y | T | Y | K | L |
| 42.8.209 | C | E | V | M | H | E | A | L | H | N | H | Y | T | Y | K | L |
| 42.8.210 | C | E | V | M | H | E | A | L | H | N | H | Y | T | Y | K | L |
| 42.8.211 | C | E | V | M | H | E | A | L | H | N | H | Y | T | Y | K | L |
| 42.8.212 | C | E | V | M | H | E | A | L | H | N | H | Y | T | Y | K | L |
| 42.8.213 | C | E | V | M | H | E | A | L | H | N | H | Y | T | Y | K | L |
| 42.8.214 | C | E | V | M | H | E | A | L | H | N | H | Y | T | Y | K | L |
| 42.8.215 | C | E | V | M | H | E | A | L | H | N | H | Y | T | Y | K | L |
| 42.8.216 | C | E | V | M | H | E | A | L | H | N | H | Y | T | Y | K | L |
| 6.5.11.1 | C | E | V | M | H | E | A | L | H | N | H | Y | T | Y | K | L |
| 6.5.11.7.122 | C | E | V | M | H | E | A | L | H | N | H | Y | T | Y | K | L |
| 42.2.19 | C | E | V | M | H | E | A | L | H | N | H | Y | T | Y | K | L |
| 42.2.7-2 | C | E | V | M | H | E | A | L | E | G | H | Y | T | Y | K | L |
| 42.2.3-5 | C | E | V | M | H | E | A | L | H | N | H | Y | T | Y | K | L |
| 42.2.3-1H | C | E | V | M | H | E | A | L | H | N | H | Y | T | Y | K | L |
| Wild-type | C | S | V | M | H | E | A | L | H | N | H | Y | T | Q | K | S |
| 42.2.2-10 | C | E | V | M | H | E | A | L | H | N | H | Y | T | Y | K | L |
| 42.2.1-20 | C | E | V | M | H | E | A | L | H | N | H | Y | T | Y | K | L |
| 6.5.11.5.42 | C | E | V | M | H | E | A | L | H | N | H | Y | T | Y | K | L |

Clones were expressed in HEK293 cells. Supernatants were captured for SPR by GE Healthcare anti-human Fab capture kit and the affinity to human and cyno TfR apical domain was measured (Table 30). The clones in Table 30 were bivalent and each TfR-binding Fc polypeptide also contained P329G and LALA mutations. The clones displayed an estimated affinity around 293-10432 nM, with very weak cross-reactivity to cyno TfR apical domain.

TABLE 30

| | K_{D} for huTfR | K_{D} for cyTfR |
|---|---|---|
| Fc Dimer | Apical Domain (M) | Apical Domain (M) |
| 42.8.9 | 1.85E−06 | 1.43E−05 |
| 42.8.10 | 3.76E−06 | 1.11E−05 |
| 42.8.15 | 5.96E−06 | 2.70E−05 |
| 42.8.16 | 7.81E−06 | |
| 42.8.17 | 1.04E−05 | |
| 42.2 | 1.00E−06 | 7.50E−06 |
| 42.8.55 | 6.16E−06 | |
| 42.8.56 | 4.83E−06 | 2.84E−05 |
| 42.8.57 | 4.90E−06 | n/a |
| 42.8.63 | 4.19E−06 | 4.94E−06 |
| 42.8.64 | 5.69E−06 | |
| 42.8.65 | 3.94E−06 | 1.03E−05 |
| 42.8.71 | 3.00E−06 | 1.29E−05 |
| 42.8.72 | 2.71E−06 | 1.34E−05 |
| 42.8.77 | 8.10E−06 | |
| 42.8.78 | 9.72E−07 | 6.74E−06 |
| 42.8.79 | 3.07E−06 | 2.86E−05 |
| 42.8.80 | 2.38E−06 | 1.36E−05 |
| 42.8.87 | 1.66E−06 | |
| 42.8.88 | 9.16E−06 | |
| 42.8.93 | 5.00E−06 | 1.76E−05 |
| 42.8.94 | 4.07E−06 | 2.22E−05 |
| 42.8.95 | 4.13E−06 | 2.31E−05 |
| 42.8.96 | 9.44E−06 | 2.19E−05 |
| 42.8.99 | 6.38E−06 | |
| 42.8.109 | 2.02E−06 | 1.36E−05 |
| 42.8.110 | 1.76E−06 | 1.58E−05 |
| 42.8.111 | 1.88E−06 | 1.91E−05 |
| 42.8.112 | 1.14E−06 | 1.33E−05 |
| 42.8.113 | 1.61E−06 | 3.43E−05 |
| 42.8.114 | 1.81E−06 | 1.83E−05 |
| 42.8.118 | 8.67E−07 | 4.29E−06 |
| 42.8.122 | 3.91E−06 | 9.84E−06 |
| 42.8.127 | 6.33E−07 | 3.04E−06 |
| 42.8.128 | 6.54E−07 | 5.45E−06 |
| 42.8.129 | 8.58E−07 | 3.14E−06 |
| 42.8.134 | 1.24E−06 | 1.32E−05 |
| 42.8.137 | 1.18E−06 | 1.26E−05 |
| 42.8.139 | 8.50E−07 | 6.10E−06 |
| 42.8.140 | 6.64E−07 | 5.23E−06 |
| 42.8.142 | 6.65E−07 | 4.63E−06 |
| 42.8.143 | 4.54E−07 | 5.18E−06 |
| 42.8.144 | 4.70E−07 | 3.87E−06 |
| 42.8.145 | 1.01E−06 | 1.14E−05 |
| 42.8.147 | 3.51E−06 | 8.03E−06 |
| 42.8.149 | 2.38E−06 | 1.60E−05 |
| 42.8.150 | 2.65E−06 | 1.18E−05 |
| 42.8.151 | 1.51E−06 | 1.31E−05 |
| 42.8.152 | 1.02E−06 | 1.09E−05 |
| 42.8.153 | 1.84E−06 | 1.45E−05 |
| 42.8.156 | 1.39E−06 | 1.33E−05 |
| 42.8.157 | 2.10E−06 | 1.55E−05 |
| 42.8.159 | 1.32E−06 | 1.08E−05 |
| 42.8.160 | 1.05E−06 | 1.16E−05 |
| 42.8.161 | 1.14E−06 | 1.05E−05 |
| 42.8.162 | 1.19E−06 | 2.32E−05 |
| 42.8.163 | 1.38E−06 | 7.82E−06 |
| 42.8.164 | 8.32E−07 | 7.45E−06 |
| 42.8.165 | 1.03E−06 | 4.46E−06 |
| 42.8.166 | 8.55E−07 | 5.00E−06 |
| 42.8.167 | 5.02E−07 | 6.59E−06 |
| 42.8.168 | 5.31E−07 | 5.70E−06 |
| 42.8.170 | 1.15E−06 | 1.19E−05 |
| 42.8.172 | 7.95E−07 | |
| 42.8.173 | 6.03E−07 | 6.72E−06 |
| 42.8.174 | 7.26E−07 | 7.78E−06 |
| 42.8.175 | 1.05E−06 | 1.58E−05 |
| 42.8.176 | 6.13E−07 | 9.78E−06 |
| 42.2.1.2 | 5.07E−07 | 9.14E−06 |
| 42.8.180 | 7.05E−07 | 7.91E−06 |
| 42.8.181 | 2.78E−06 | 1.21E−05 |
| 42.8.182 | 2.29E−06 | 1.44E−05 |
| 42.8.183 | 2.33E−06 | 1.51E−05 |
| 42.8.184 | 1.68E−06 | 1.70E−05 |
| 42.8.185 | 1.85E−06 | 1.33E−05 |
| 42.8.186 | 2.05E−06 | 2.22E−05 |
| 42.8.187 | 1.60E−06 | 1.34E−05 |
| 42.8.188 | 1.03E−06 | 1.25E−05 |
| 42.8.189 | 1.10E−06 | 1.35E−05 |
| 42.8.190 | 1.31E−06 | 9.78E−06 |
| 42.8.191 | 5.98E−07 | 8.14E−06 |
| 42.8.192 | 7.61E−07 | 1.13E−05 |
| 42.8.193 | 1.60E−06 | 1.33E−05 |
| 42.8.194 | 1.23E−06 | 1.18E−05 |
| 42.8.195 | 1.23E−06 | 1.33E−05 |
| 42.8.196 | 9.24E−07 | 9.34E−06 |
| 42.8.197 | 7.55E−07 | 1.14E−05 |
| 42.8.198 | 8.41E−07 | 1.22E−05 |
| 42.8.199 | 8.69E−07 | 8.05E−06 |
| 42.8.200 | 5.05E−07 | 6.21E−06 |
| 42.8.201 | 6.47E−07 | 4.91E−06 |
| 42.8.202 | 5.05E−07 | 6.27E−06 |
| 42.8.203 | 3.94E−07 | 5.46E−06 |
| 42.8.204 | 4.36E−07 | 4.63E−06 |
| 42.8.205 | 1.13E−06 | 1.14E−05 |
| 42.8.206 | 8.27E−07 | 1.08E−05 |
| 42.8.207 | 1.20E−06 | 1.38E−05 |
| 42.8.208 | 4.46E−07 | 7.41E−06 |
| 42.8.209 | 3.80E−07 | 6.03E−06 |
| 42.8.210 | 5.96E−07 | 7.88E−06 |
| 42.8.211 | 9.66E−07 | 1.07E−05 |
| 42.8.212 | 6.20E−07 | 9.37E−06 |
| 42.8.213 | 8.72E−07 | 9.79E−06 |
| 42.8.214 | 4.27E−07 | 6.09E−06 |
| 42.8.215 | 2.93E−07 | 4.94E−06 |
| 42.8.216 | 4.21E−07 | 5.85E−06 |
| 6.5.11.1 | 1.00E−05 | |
| 6.5.11.7.122 | 4.60E−0.6 | |
| 42.2.19 | 8.00E−08 | 1.00E−06 |
| 42.2.7−2 | 1.60E−07 | 2.50E−06 |
| 42.2.3−5 | 2.60E−07 | 1.80E−06 |
| 42.2.3−1H | 2.20E−07 | 2.00E−06 |
| 42.2.2−10 | 5.00E−08 | 1.00E−06 |
| 42.2.1−20 | 4.40E−07 | 7.00E−06 |
| 6.5.11.5.42 | 2.00E−06 | |

### Example 12. PK Evaluation

Clones 42.8.17, 42.8.15, 42.8.80, 42.8.196, 42.2.3-1H, and 42.2.19 in monovalent and 42.2.1.2 bivalent forms were tested for their PK in huTfR^{appical} knock-in mice to ensure that there are no PK liabilities. The monovalent clones used here contained the TfR-binding site with T366W knob, P329G, and LALA mutations as the first Fc polypeptide and Fc sequence with T366S, L368A, and Y407V hole, P329G, and LALA mutations as the second Fc polypeptide. Both of Fc polypeptides in bivalent clones used here contained the TfR-binding site with P329G and LALA mutations.

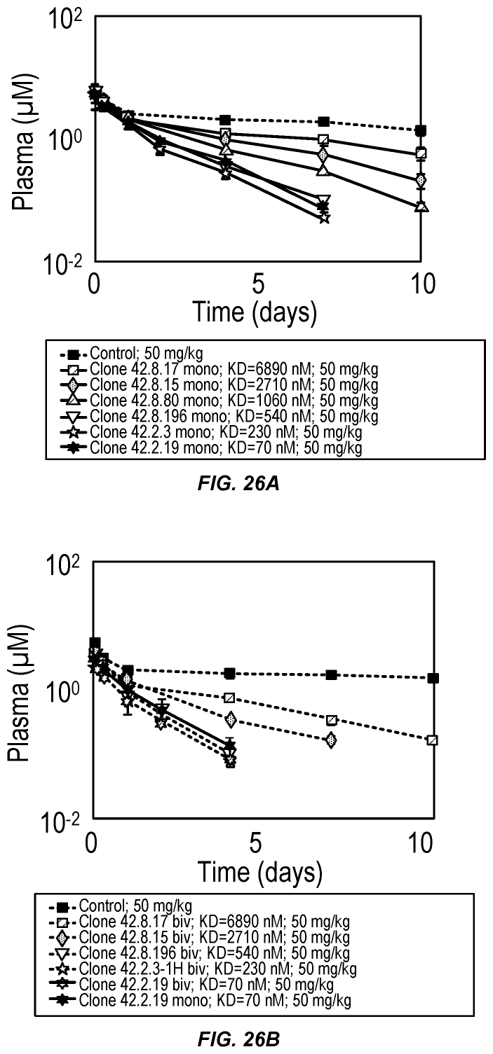

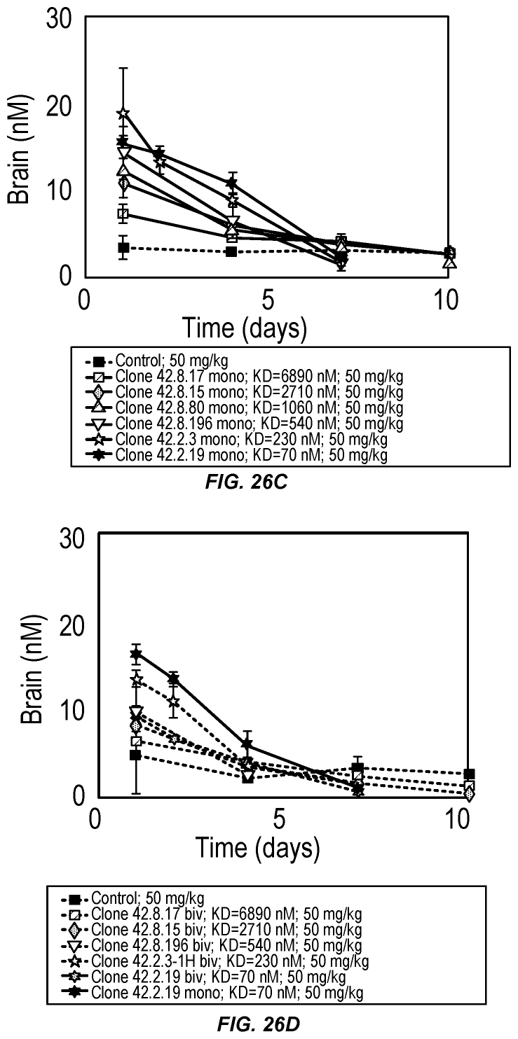

The clones and the controls were dosed intravenously into huTfR^{apical} knock-in mice at 50 mg/kg. Brain and plasma concentrations were measured at 24 hours (FIGS. 26A-26D). Plasma PK showed a clearance of all TfR-binding clones compared to the control as expected (FIGS. 26A and 26B). Brain concentration of the clones had sustained brain concentrations beyond 10 days(FIGS. 26C and 26D).

### Example 13. Plasma and Brain PK of Monovalent Cd98Hc Affinity Variants to 21 Days

To further study plasma and brain PK of CD98hc-binding molecules, monovalent CD98hc LLB2-10-8 affinity variants ranging from 20 nM to S50 nM, CD98hc^{mu/hu} KI mice were administered a single 50 mg/kg dose via IV according to the groups in Table 31A below. Mice were sacrificed on day 1, 2, 7, 14, or 21 post dose and blood and brain tissue were collected.

TABLE 31A
Study Design

| | | | | | | Terminal | | |
|---|---|---|---|---|---|---|---|---|
| | | CD98hc | | | | time | | Dose |
| Clone | | binding | | EF | Affinity | points | | IV |
| # | Clone Name | valency | Fabs | status | (nM) | (days) | N | (mg/kg) |
| Clone | LLB2.10.8.14.3 | Monovalent | NBF | + | 20 | 1, 2, 7, | 5 per | 50 |
| 22 | | | | | | 14, 21 | timepoint | |
| Clone | LLB2.10.8.10.3 | Monovalent | NBF | + | 43 | 1, 2, 7, | 5 per | 50 |
| 24 | | | | | | 14, 21 | timepoint | |
| Clone | LLB2.10.8.10.8 | Monovalent | NBF | + | 94 | 1, 2, 7, | 5 per | 50 |
| 25 | | | | | | 14, 21 | timepoint | |
| Clone | LLB2-10-8-d3 | Monovalent | NBF | + | 275 | 1, 2, 7, | 5 per | 50 |
| 29 | | | | | | 14, 21 | timepoint | |
| Clone | LLB2-10-8-d6 | Monovalent | NBF | + | 550 | 1, 2, 7, | 5 per | 50 |
| 30 | | | | | | 14, 21 | timepoint | |
| Ctrl 1 | Non-binding | — | NBF | + | N/A | 1, 2, 7, | 5 per | 50 |
| | Fab (negative | | | | | 14,21 | timepoint | |
| | control) | | | | | | | |

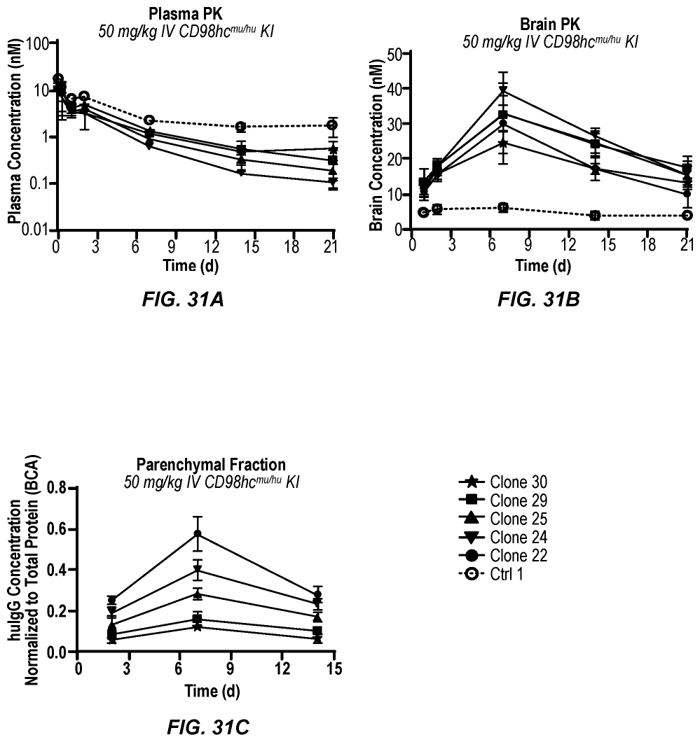

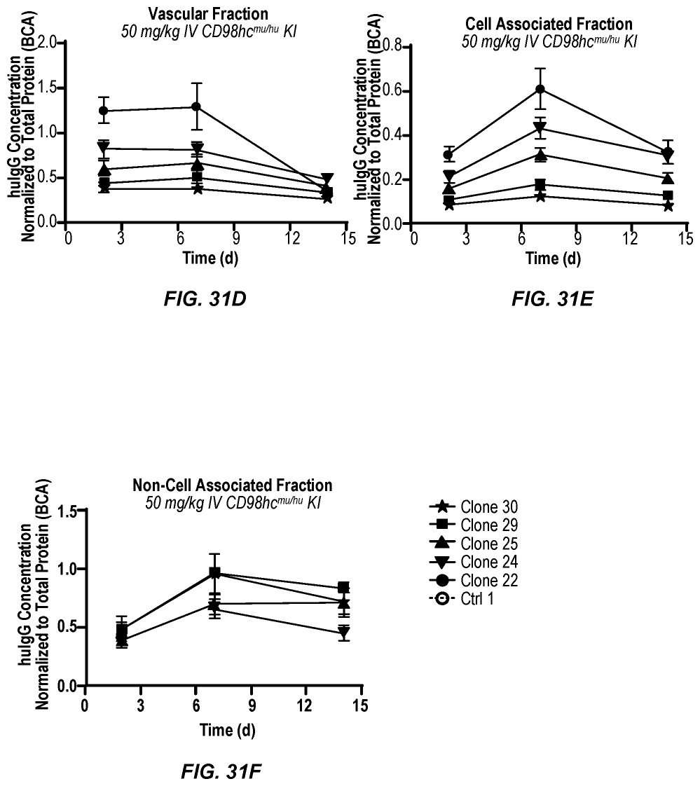

huIgG PK in plasma and whole brain lysate out to 21 days post dose was assessed following the protocol for sample collection and analysis as described above (i.e., Example 4 for plasma PK and Example 5 for brain PK). Results are shown in FIGS. 31A and 31B. There was a correlation between stronger affinity for CD98hc-binding molecules and faster clearance from plasma (FIG. 31A). Increased huIgG concentrations were observed for all huCD98hc-binding molecules compared to a control non-binding IgG (FIG. 31B). Brain concentrations of LLB2 CD98hc-binding molecules increased until 7 days post dose and levels remained higher than the control IgG at 21 days post dose. In these experiments, compared to TfR binding molecules, CD98hc molecules have a delayed Tₘₐₓ and concentrations remain higher in brain than control antibodies for longer. Slightly lower AUCs were observed for the strongest and lowest affinity clones.

Furthermore, capillary depletion (performed via the protocol described above in Example 5) demonstrated that all monovalent LL2 affinity variants crossed the BBB into the brain parenchyma (FIGS. 31C and 31D). There were lower concentrations of weaker affinity LLB32 variants in the parenchymal fraction despite similar Cₘₐₓ concentrations. Higher concentrations of the weaker affinity variants were detected in the non-cell associated fraction, suggesting they substantially contribute to whole brain huIgG (FIG. 31E). There was correlation between higher affinity and higher huIgG concentrations in the cell-associated fraction (FIG. 31F). All huIgG values were normalized to total protein concentration measured by BCA. The control IgG was below LLOQ in all fractions.

### Example 14. Plasma and Brain PK of Bivalent CD98HC Affinity Variants to 28 Days

To further study plasma and brain PK of CD98hc-binding molecules, bivalent CD98hc LLB2-10-8 affinity variants ranging from 275 nM to 2100 nM were administered to CD98hc^{mu/hu} KI mice were at a single 50 mg/kg dose via IV according to the groups in Table 3lB below. Mice were sacrificed on day 1, 2, 7, 14, or 28 post dose and blood and brain tissue were collected.

TABLE 31B
Study Design

| | | | | | | Terminal | | |
|---|---|---|---|---|---|---|---|---|
| | | CD98hc | | | | time | | Dose |
| Clone | Clone | binding | | EF | Affinity | points | | IV |
| # | Name | valency | Fabs | status | (nM) | (days) | N | (mg/kg) |
| Clone | LLB2-10-8- | Bivalent | NBF | + | 280 | 1, 2, 7, | 5 per | 50 |
| 31 | d1 | | | | | 14, 28 | timepoint | |
| Clone | LLB2-10-8- | Bivalent | NBF | + | 275 | 1, 2, 7, | 5 per | 50 |
| 32 | d3 | | | | | 14, 28 | timepoint | |
| Clone | LLB2-10-8- | Bivalent | NBF | + | 550 | 1, 2, 7, | 5 per | 50 |
| 35 | d18 | | | | | 14, 28 | timepoint | |
| Clone | LLB2-10-8- | Bivalent | NBF | + | 550 | 1, 2, 7, | 5 per | 50 |
| 33 | d6 | | | | | 14, 28 | timepoint | |
| Clone | LLB2-10-8- | Bivalent | NBF | + | 2100 | 1, 2, 7, | 5 per | 50 |
| 34 | d12 | | | | | 14, 28 | timepoint | |
| Ctrl 1 | Non- | — | NBF | + | N/A | 1, 2, 7, | 5 per | 50 |
| | binding Fab | | | | | 14, 28 | timepoint | |
| | (negative | | | | | | | |
| | control) | | | | | | | |

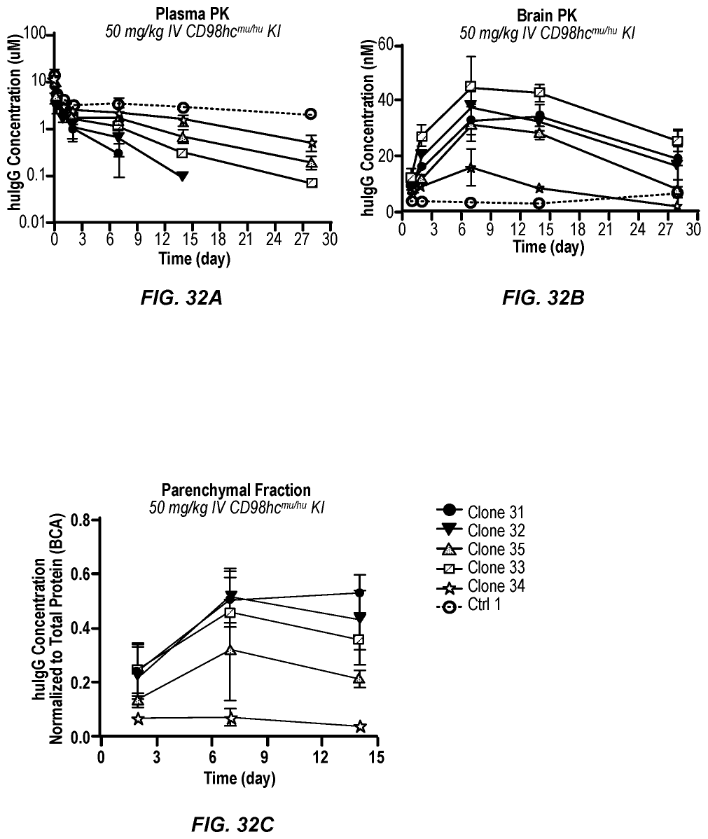

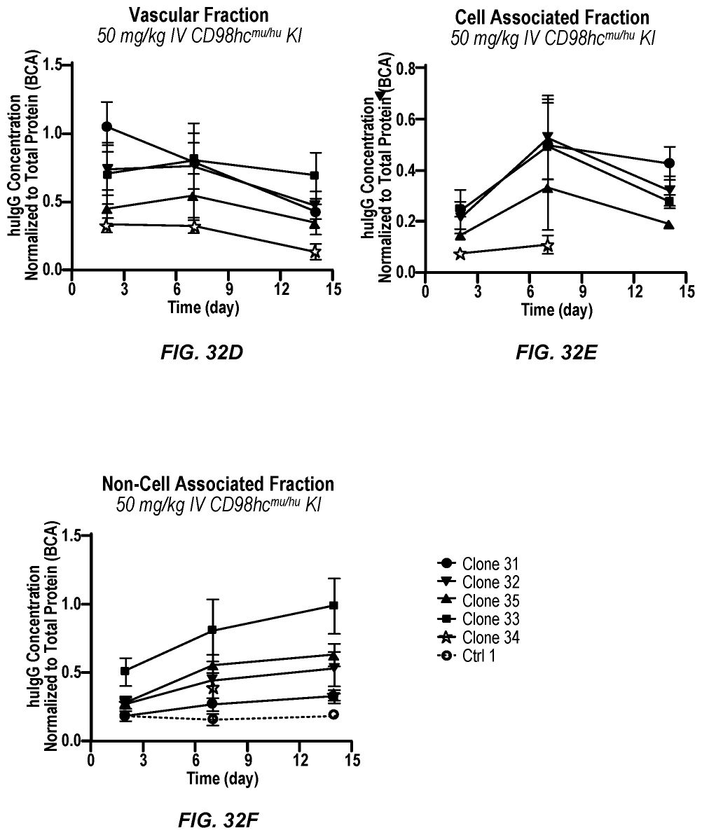

huIgG PK in plasma and whole brain lysate out to 28 days post dose was assessed following the protocol for sample collection and analysis as described above (i.e., Example 4 for plasma PK and Example 5 for brain PK). Given a trend of higher brain exposure for bivalent CD98hc-binding molecules in FIG. 12B, we hypothesized that bivalent CD98hc molecules could have an even longer brain exposure than monovalent molecules, so the last time point of the study was set for 28 days (instead of 21 days). Results are shown in FIGS. 32A and 32B. There was a correlation between stronger affinity for CD98hc-binding molecules and faster clearance from plasma (FIG. 32A). Increased huIgG concentrations were observed for all huCD98hc-binding molecules compared to a control non-binding IgG (FIG. 32B). Brain concentrations of LLB2 TVs increased until 7 days post dose and levels, and for all clones except the weakest affinity, remained higher than the control IgG at 28 days post dose. Similar to monovalent CD98hc TVs, bivalent CD98hc TVs have a delayed Tₘₐₓ and concentrations in brain remain elevated for longer than TfR TVs.

Furthermore, capillary depletion (performed via the protocol described above in Example 5) demonstrated that all bivalent LLB2 affinity variants crossed the BBB into the brain parenchyma (FIGS. 32C and 32D). Higher concentrations of the weaker affinity variants were detected in the non-cell associated fraction (FIG. 32E). Other than the weakest affinity clone, all bivalent LLB2 variants were detected in the cell associated fraction (FIG. 32F). All huIgG values were normalized to total protein concentration measured by BCA. The control IgG was below LLOQ in all fractions except the non-cell associated fraction.

### Example 15. Plasma and Brain PK of Affinity Matched LLB1 and LLB2 Variants

In order to elucidate any differences between affinity matched monovalent LLB1 and LLB2 variants on plasma and brain pharmacokinetics (PK), CD98hc^{mu/hu} KI mice were administered a 50 mg/kg IV dose according to the groups below in Table 31C. Mice were sacrificed on day 1, 2, 4, 10, or 21 post dose and blood and brain tissue were collected.

TABLE 31C
Study Design

| | | | | | | Terminal | | |
|---|---|---|---|---|---|---|---|---|
| | | CD98hc | | | | time | | Dose |
| Clone | Clone | binding | | EF | Affinity | points | | IV |
| # | Name | valency | Fabs | status | (nM) | (days) | N | (mg/kg) |
| Clone | LLB1-3- | Monovalent | NBF | + | 175 | 1, 2, 4, | 5 per | 50 |
| 14 | 16-2 | | | | | 10, 21 | timepoint | |
| Clone 8 | LLB2-10-8 | Monovalent | NBF | + | 100-200 | 1, 2, 4, | 5 per | 50 |
| | | | | | | 10, 21 | timepoint | |
| Ctrl 1 | Non- | — | NBF | + | N/A | 1, 2, 4, | 5 per | 50 |
| | binding Fab | | | | | 10, 21 | timepoint | |
| | (negative | | | | | | | |
| | control) | | | | | | | |

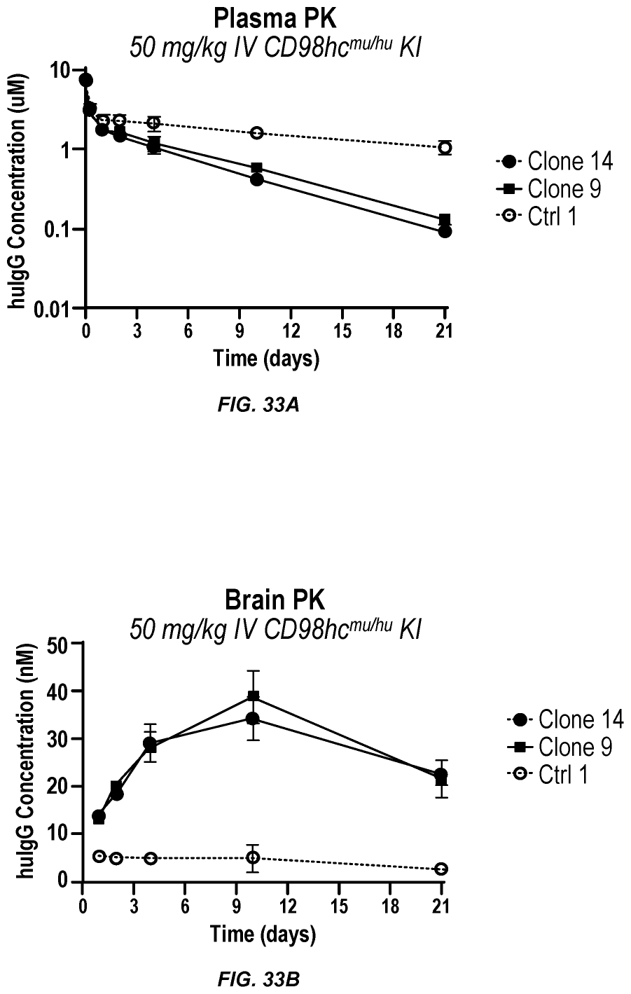

huIgG PK in plasma and whole brain lysate out to 21 days post dose was assessed following the protocol for sample collection and analysis as described above (i.e., Example 4 for plasma PK and Example 5 for brain PK). Results are shown in FIGS. 33A and 33. LLB1 and LL2 variants had similar plasma clearance (FIG. 33A). huIgG PK in whole brain lysate. LLB1 and LL2 have similar brain exposure across all tested timepoints (FIG. 33B). No plasma or brain PK differences were observed in affinity matched monovalent LLB 1 and LL2 variants.

### Example 16. Plasma and Brain PK and CNS and Cell Type Biodistribution for Cd98Hc LLB2-10-8 Variants (Monovalent and Bivalent; Effector Positive and Negative)

To further elucidate differences in PK and brain biodistribution for monovalent and bivalent CD98hc LLB32-10⁻⁸ with and without effector function modifying mutations, CD98hc^{mu/hu} KI mice were administered a single 50 mg/kg dose via IV according to the groups in Table 31D below. Mice were sacrificed on day 1, 4, 7, 14, or 21 post dose and blood and brain tissue were collected.

TABLE 31D
Study Design

| | | | | | | Terminal | | |
|---|---|---|---|---|---|---|---|---|
| | | CD98hc | | | | time | | |
| Clone | Clone | binding | | EF | Affinity | points | | Dose IV |
| # | Name | valency | Fabs | status | (nM) | (days) | N | (mg/kg) |
| Clone | LLB2-10-8 | Monovalent | NBF | − | 100-200 | 1, 4, 7, | 5 per | 50 |
| 27 | | | | | | 14, 21 | timepoint | |
| Clone | LLB2-10-8 | Bivalent | NBF | − | 100-200 | 1, 4, 7, | 5 per | 50 |
| 36 | | | | | | 14,21 | timepoint | |
| Clone | LLB2-10-8 | Monovalent | NBF | + | 100-200 | 1, 4, 7, | 5 per | 50 |
| 9 | | | | | | 14,21 | timepoint | |
| Clone | LLB2-10-8 | Bivalent | NBF | + | 100-200 | 1, 4, 7, | 5 per | 50 |
| 26 | | | | | | 14, 21 | timepoint | |
| Ctrl 1 | Non- | — | NBF | + | N/A | 1, 4, 7, | 5 per | 50 |
| | binding | | | | | 14, 21 | timepoint | |
| | Fab | | | | | | | |
| | (negative | | | | | | | |
| | control) | | | | | | | |

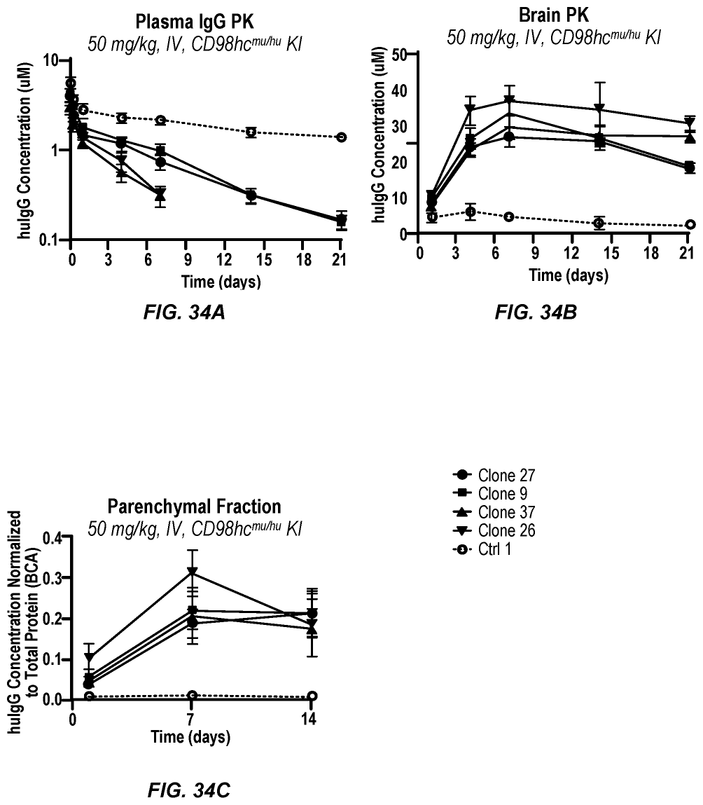

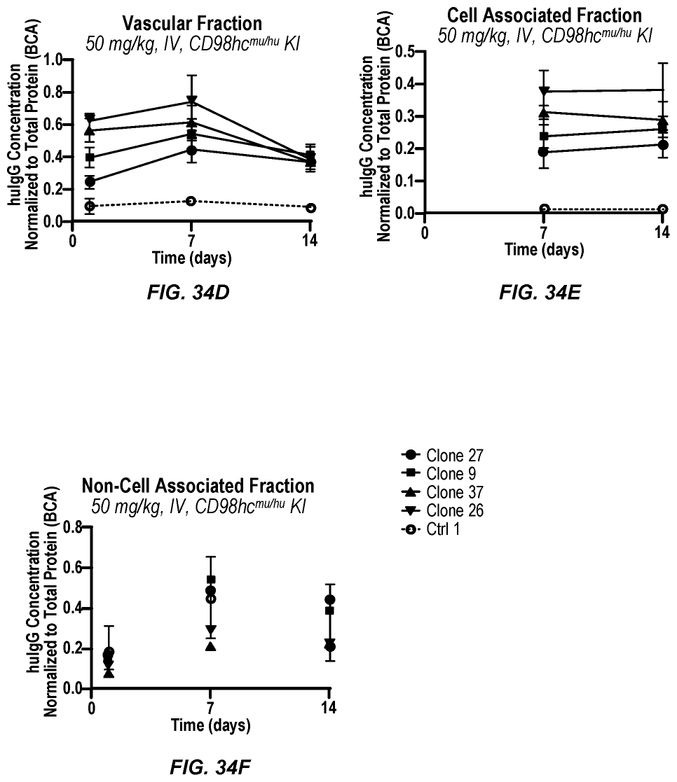

huIgG PK in plasma and whole brain lysate out to 21 days post dose was assessed following the protocol for sample collection and analysis as described above (i.e., Example 4 for plasma PK and Example 5 for brain PK). Results are shown in FIGS. 34A and 34B. The bivalent variants cleared faster from plasma than monovalent variants and effector function mutations did not impact plasma clearance (FIG. 34A). Increased huIgG concentrations were observed for all huCD98hc-binding molecules compared to the control non-binding IgG (FIG. 34B). Brain concentrations in all tested variants were similar until later timepoints when the bivalent variants retrained higher concentrations in brain than monovalent variants. The was a small increase in brain exposure for the bivalent LLB2 TV with wildtype effector function, i.e., effector positive, but no impact on effector function mutants, i.e., effector negative, for monovalent LLB2.

Furthermore, capillary depletion (performed via the protocol described above in Example 5) demonstrated that all LLB2 variants crossed the BBB into the brain parenchyma (FIGS. 34C and 34D). Monovalent LLB2 variants were found at higher concentrations in the non-cell associated fraction whereas bivalent LLB2 variants were found at higher concentrations in the cell associated fraction (FIGS. 34E and 34F). All huIgG values were normalized to total protein concentration measured by BCA. Consistent with data suggesting that higher CD98hc affinity results in molecules being more cell associated, these data suggest that high valency for CD98hc binding also results in molecules being more cell associated.

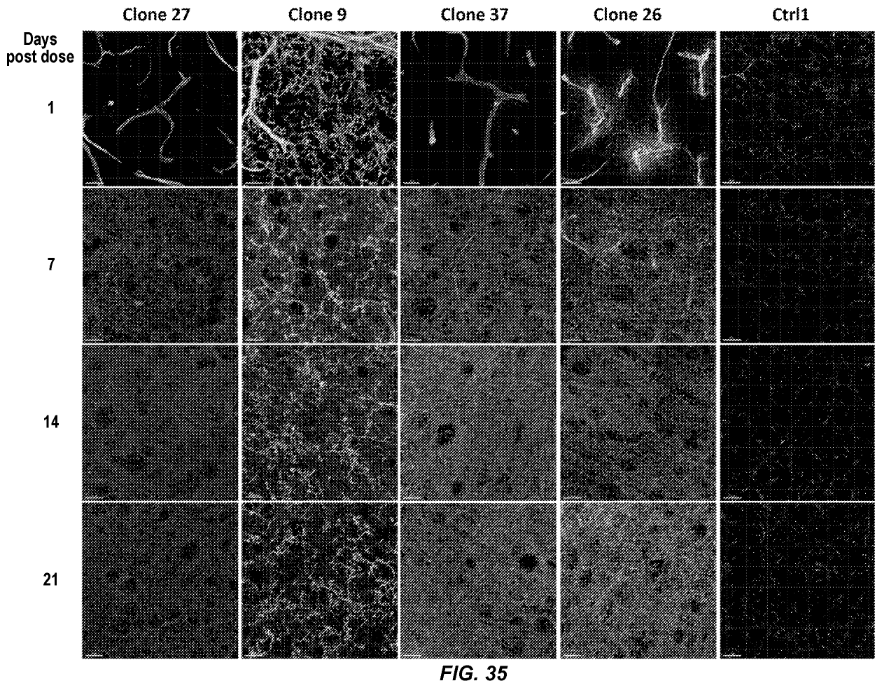

Additionally, immunohistochemistry for huIgG on brain sections from CD98hc^{mu/hu} KI mice 1, 7, 14 and 21 days post-dose was performed according to the protocols described above (i.e., in Example 6). Results are shown in FIG. 35. There is an increase in parenchymal huIgG staining between 1 and 7 days, consistent with the huIgG ELISA quantification on whole brain lysate and the parenchymal capillary depletion fraction. Monovalent LLB2 with WT effector function, i.e., effector positive, had a prominently glial localization whereas bivalent LLB2, regardless of effector function status, and monovalent LLB2 with LALAPG, i.e., effector negative mutant, had a prominent diffuse punctate staining. Therefore, monovalent LLB2 with WT effector function had distinct CNS biodistribution compared to other versions suggesting that the combination of monovalent CD98hc and FcγR binding drives localization of these molecules to microglia.

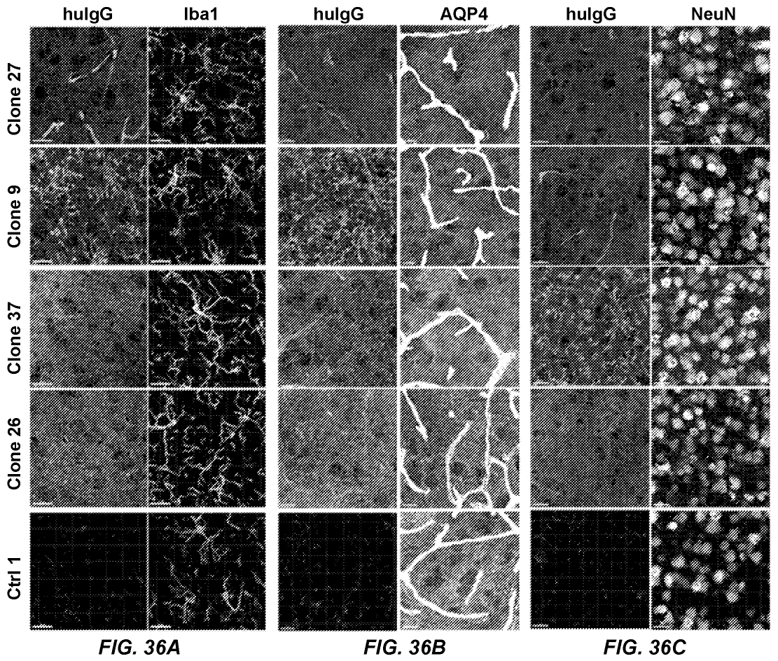

Immunohistochemistry for huIgG and CNS cell type markers (i.e., Iba1 for microglial, AQP4 for astrocyte processes, and NeuN for neurons) was performed on brain sections from CD98hc^{mu/hu} KI 7 days post-dose was performed according to the protocols described above (i.e., in Example 6). Results are shown in FIGS. 36A-36C. Monovalent LLB2 with WT effector function had a prominently microglial localization whereas bivalent LLB2, regardless of effector function status, and monovalent LLB2 with LALAPG had a prominent colocalization with AQP4, consistent with localization to astrocytes. LLB2 variants were not observed to localize to neurons.

One interpretation of these data is that the delayed peak brain concentration (Cₘₐₓ) observed above in Examples 16 and 17 is due a slower rate of internalization (compared to TfR binding molecules). Slow cellular internalization could also contribute to the CD98hc TV being found in the non-cellular fraction. This interpretation is consistent with the observation that increasing the probability of CD98hc-binding molecules disengaging via weaker affinity and/or monovalency, increases the proportion of CD98hc-binding molecules found in non-cell associated brain fractions.

### Example 17. PK, PD, and CNS Biodistribution of Cd98Hc TVs with BACE1 Fabs

To study the effects of binding Fabs on PK, PD, and CNS biodistribution of CD98hc molecules, monovalent and bivalent LLB2-10-8 TVs were constructed with anti-BACE1 Fabs. CD98hc^{mu/hu} KI mice were administered a single 50 mg/kg dose via IV according to the groups in Table 31E below. Mice were sacrificed on day 1, 4, 7, 14, or 21 post dose and blood and brain tissue were collected.

TABLE 31E
Study Design

| | | | | | | Terminal | | |
|---|---|---|---|---|---|---|---|---|
| | | CD98hc | | | | time | | |
| Clone | Clone | binding | | EF | Affinity | points | | Dose IV |
| # | Name | valency | Fabs | status | (nM) | (days) | N | (mg/kg) |
| Clone | LLB2-10- | Monovalent | BACE1 | + | 100-200 | 1, 4, 7, | 5 per | 50 |
| 40 | 8 | | | | | 14, 21 | timepoint | |
| Clone | LLB2-10- | Bivalent | BACE1 | + | 100-200 | 1, 4, 7, | 5 per | 50 |
| 41 | 8 | | | | | 14, 21 | timepoint | |
| Ctrl 3 | Anti- | — | BACE1 | + | N/A | 1, 4, 7, | 5 per | 50 |
| | BACE1 | | | | | 14, 21 | timepoint | |

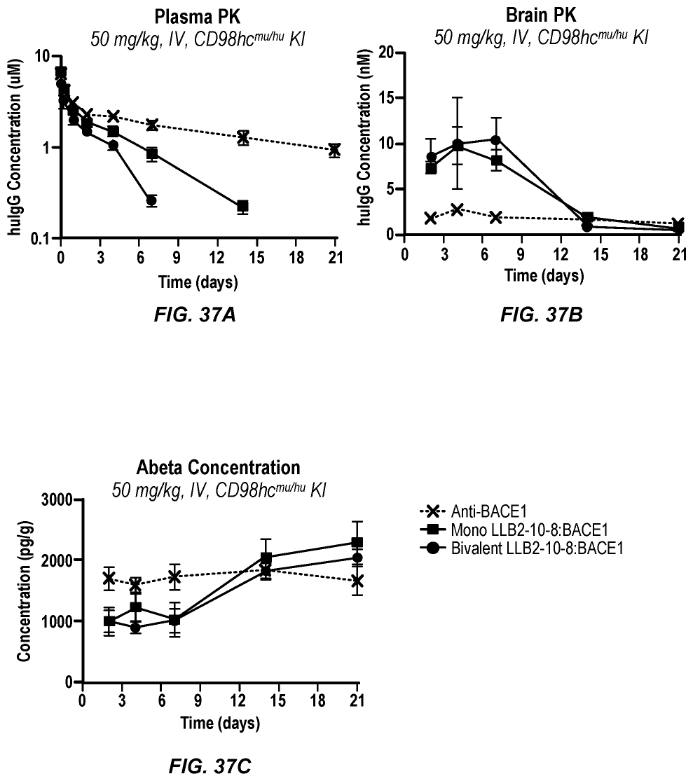

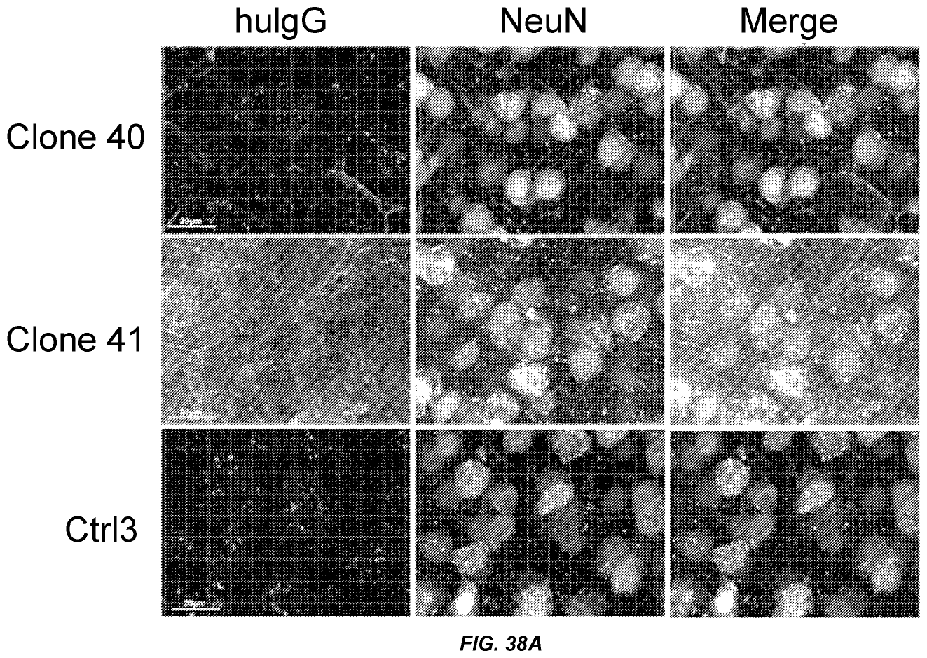

huIgG PK in plasma and whole brain lysate out to 21 days post dose was assessed following the protocol for sample collection and analysis as described above (i.e., Example 4 for plasma PK and Example 5 for brain PK). Results are shown in FIGS. 37A and 37B. huIgG PK in plasma out to 21 days post dose showed that bivalent CD98hc binding resulted in faster clearance from plasma (FIG. 37A). This was consistent with studies with non-binding Fabs. In brain, consistent with studies with non-binding Fabs, we observed Tₘₐₓ between 4-7 days with ATV.CD98hc:BACE molecules, but the maximum concentration of ATV.CD98hc:BACE1 was lower than CD98hc TVs with non-binding Fabs. Additionally, ATV.CD98hc:BACE1 cleared from brain faster and concentrations were similar to the negative control antibody by 14 days post dose. This contrasts with the greater than 21-day brain exposure we observed with CD98hc TVs with non-binding Fabs. These data suggest that BACE1 binding resulted in less retention in brain than was observed with non-binding Fabs (FIG. 37B). This was likely due to BACE1 binding directing CD98hc to neurons and specifically lysosomes within neurons resulting in degradation of the LLB2:BACE1 molecules. These data also suggest that brain exposure of CD98hc TV paired with different Fabs should be assessed on a target by target basis.

BACEl inhibition of amyloid precursor protein APP cleavage was used as a pharmacodynamic readout of antibody activity in brain. Brain tissue was homogenized in 10× tissue weight of 5M guanidine-HCl and then diluted 1:10 in 0.25% casein buffer in PBS. Mouse Aβ40 levels in brain lysate were measured using a sandwich ELISA. A 384-well MaxiSorp plate was coated overnight with a polyclonal capture antibody specific for the C-terminus of the Aβ40 peptide (Millipore #ABN240). Casein-diluted guanidine brain lysates were further diluted 1:2 on the ELISA plate and added concurrently with the detection antibody, biotinylated anti-mouse/rat β-Amyloid M3.2. Samples were incubated overnight at 4° C. prior to addition of streptavidin-TRP followed by TMB substrate. The standard curve, 0.78-50 pg/mL msAβ40, was fit using a four-parameter logistic regression.

Aβ40 measurements demonstrated that BACE1 inhibition occurred at 1, 4, and 7 days post dose, consistent with when LLB2:BACE1 molecule concentrations were greater than the control anti-BACE1 antibody (FIG. 37C).

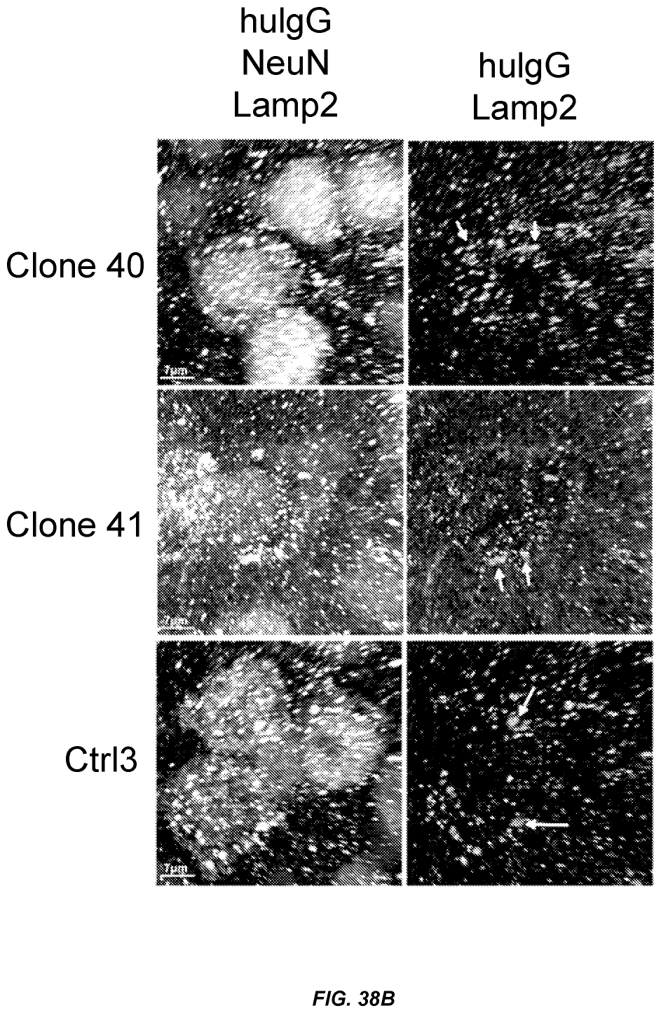

Additionally, immunohistochemistry for huIgG, NeuN (neurons), and LAMP2 (lysosomes) was performed according to the protocols described above (i.e., in Example 6) on brain sections from CD98hc^{mu/hu} KI 7 days post 50mpk dose of monovalent and bivalent LLB2:BACE1 molecules. The results are shown in FIGS. 38A and 38B. Monovalent LLB2:BACE1 had predominant localization to neurons. Bivalent LLB2:BACE1 also had some localization to neurons as well as diffuse puncta likely consistent with CD98hc driven localization to astrocyte processes. The anti-BACE1 control also showed localization to neurons (FIG. 38A). All 3 molecules tested showed colocalization with Lamp2 positive lysosomes in neurons consistent with BACE1 localization in the endolysosomal pathway (FIG. 381B). This data suggest BACE1 drives CD98hc-binding molecules to degradation in the lysosomes of neurons. These data suggest that monovalent CD98hc TVs can be directed away from CD98hc by binding to other proteins, such as therapeutic targets. For instance, FcγR binding seems to drive monovalent LLB32 molecules to localize with microglia (see Example 10), while as discussed above, neuronal anti-BACEl Fabs drives the monovalent LLB2 to neurons. Bivalent CD98hc TVs retain some CD98hc driven biodistribution.

### Example 18. Plasma and Brain PK for 15 Mpk of Monovalent and Bivalent LLB2 Variants

In order to characterize plasma and brain PK of CD98hc-binding molecules at lower dose levels, we dosed monovalent and bivalent LLB32 variants with affinities ranging from 20 nM to 550 nM at 15 mg/kg IV in CD98hc^{mu/hu} KI mice according to the groups below in Tables 31F and 31G. Mice were sacrificed on day 1, 4, 7, 14, or 21 post dose and blood and brain tissue were collected.

TABLE 31F
Study Design of monovalent LLB2 variants

| | | | | | | Terminal | | |
|---|---|---|---|---|---|---|---|---|
| | | CD98hc | | | | time | | |
| Clone | Clone | binding | | EF | Affinity | points | | Dose IV |
| # | Name | valency | Fabs | status | (nM) | (days) | N | (mg/kg) |
| Clone | LLB2-10- | Monovalent | NBF | − | 20 | 1, 4, 7, | 5 per | 15 |
| 22 | 8-14-3 | | | | | 14, 21 | timepoint | |
| Clone | LLB2-10-8 | Monovalent | NBF | + | 100-200 | 1, 4, 7, | 5 per | 15 |
| 9 | | | | | | 14, 21 | timepoint | |
| Clone | LLB2-10- | Monovalent | NBF | + | 550 | 1, 4, 7, | 5 per | 15 |
| 30 | 8-d6 | | | | | 14, 21 | timepoint | |
| Ctrl 1 | Non- | — | NBF | + | N/A | 1, 4, 7, | 5 per | 15 |
| | binding | | | | | 14, 21 | timepoint | |
| | Fab | | | | | | | |
| | (negative | | | | | | | |
| | control) | | | | | | | |

TABLE 31G
Study Design of bivalent LLB2 variants

| | | | | | | Terminal | | |
|---|---|---|---|---|---|---|---|---|
| | | CD98hc | | | | time | | |
| Clone | Clone | binding | | EF | Affinity | points | | Dose IV |
| # | Name | valency | Fabs | status | (nM) | (days) | N | (mg/kg) |
| Clone | LLB2-10-8 | Bivalent | NBF | − | 100-200 | 1, 4, 7, | 5 per | 15 |
| 26 | | | | | | 14, 21 | timepoint | |
| Clone | LLB2-10-8- | Bivalent | NBF | + | 275 | 1, 4, 7, | 5 per | 15 |
| 32 | d3 | | | | | 14, 21 | timepoint | |
| Clone | LLB2-10-8- | Bivalent | NBF | + | 550 | 1, 4, 7, | 5 per | 15 |
| 33 | d6 | | | | | 14,21 | timepoint | |
| Ctrl 1 | Non- | — | NBF | + | N/A | 1, 4, 7, | 5 per | 15 |
| | binding Fab | | | | | 14, 21 | timepoint | |
| | (negative | | | | | | | |
| | control) | | | | | | | |

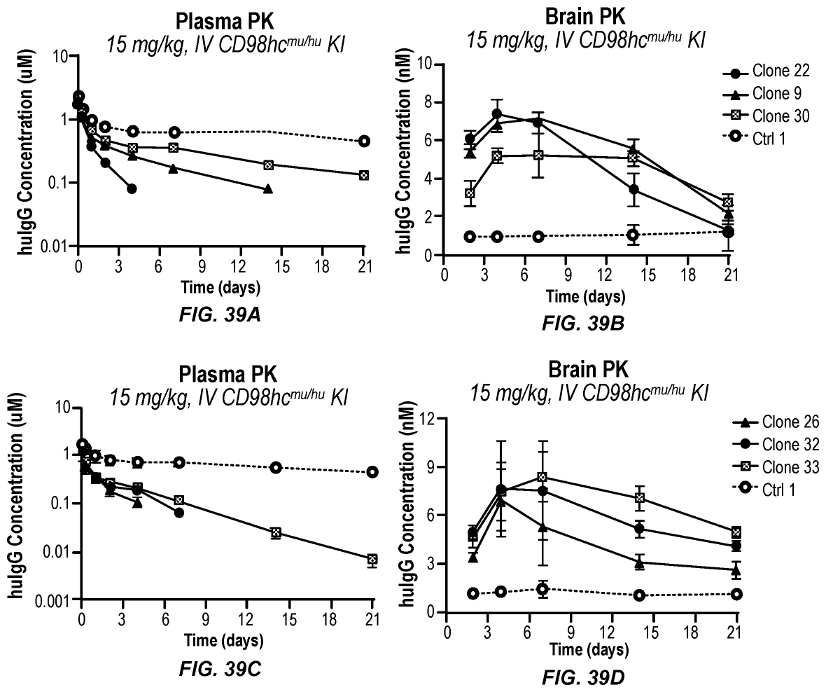

huIgG PK in plasma and whole brain lysate was assessed following the protocol for sample collection and analysis as described above (i.e., Example 4 for plasma PK and Example 5 for brain PK). Results are shown in FIGS. 39A-39D. Stronger affinity and higher valency of CD98hc-binding molecules resulted in faster clearance from plasma (FIG. 39A for monovalent and FIG. 39C for bivalent). Increased huIgG concentrations were observed for all huCD98hc-binding molecules compared to a control non-binding IgG (FIG. 39B for monovalent and FIG. 39D for bivalent). Brain concentrations of LLB2 TVs increased until 7 days post dose and levels remained higher than the control IgG, except for the highest affinity monovalent clone, at 21 days post dose. Consistent with studies with higher doses of CD98hc TV, at lower dose, CD98hc TVs also have delayed Tₘₐₓ and huIgG concentrations remain elevated for longer than TfR TVs.

### Example 19. Plasma and Brain Uptake of LLB2 and TV42 in Non-Human Primate (NHP) and Cell Type Biodistribution

In order to assess the translatability of the above data on CD98hc-binding molecules in mice, a study on plasma and brain exposure of LLB2 (CD98hc binding molecule) compared to TV42 (TfR binding molecule) was conducted. Groups of cynomolgus monkeys per Table 31H below were administered a single 30 mg/kg dose via IV. The monkeys were taken down 4 days post dose and blood and brain tissue were collected.

TABLE 31H
Study Design

| | | | | | | Terminal | | |
|---|---|---|---|---|---|---|---|---|
| | | CD98hc | | | Cyno | time | | Dose |
| Clone | Clone | binding | | EF | Affinity | points | | IV |
| # | Name | valency | Fabs | status | (nM) | (days) | N | (mg/kg) |
| | TV42.2.19 | Bivalent | NBF | − | 1000 | 4 | 3 | 30 |
| Clone | LLB2-10-8- | Bivalent | NBF | + | 450 | 4 | 3 | 30 |
| 36 | 14-3 | | | | | | | |
| Clone | LLB2-10-8- | Monovalent | NBF | − | 450 | 4 | 3 | 30 |
| 39 | 14-3 | | | | | | | |
| Clone | LLB2-10-8- | Monovalent | NBF | + | 450 | 4 | 3 | 30 |
| 22 | 14-3 | | | | | | | |
| Ctrl 1 | Non- | — | NBF | + | N/A | 4 | 3 | 30 |
| | binding Fab | | | | | | | |
| | (negative | | | | | | | |
| | control) | | | | | | | |

The total test article concentrations in monkey serum and brain lysate samples were quantified using a generic anti-human IgG sandwich-format electrochemiluminescence immunoassay (ECLIA) on a Meso Scale Discovery (MSD) platform. Briefly, 1% casein-based PBS blocking buffer (Thermo Scientific, Waltham, MA) was added to an MSD GOLD 96-well small-spot streptavidin-coated microtiter plate (Meso Scale Discovery, Rockville, MD) and incubated for approximately 1 h. Following the plate blocking and wash steps, a biotinylated goat anti-human IgG antibody (SouthernBiotech, Birmingham, AL) at a working concentration of 0.5 g/mL was added to coat the assay plate and allowed to incubate for 1-2 h. Subsequently, test samples were diluted (MRD of 1:100 in 0.5% casein-based PBS assay buffer) and added to the assay plate. Following the 1-2 h incubation in the capture step, a pre-adsorbed secondary ruthenylated (SULFO-TAG) goat anti-human IgG antibody (Meso Scale Discovery, Rockville, MD) at a working solution of 0.5 g/mL was added to the assay plate and incubated for approximately 1 h. An assay read buffer (1× MSD Read Buffer T) was then added to generate the electrochemiluminescence (ECL) assay signal, expressed in ECL units (ECLU). All of the assay reaction steps were performed at ambient temperature and with shaking on a plate shaker (where appropriate). In serum, the assay had an MRD of 100 and a dynamic calibration standard range of 19.5-2500 ng/mL in neat matrix with 8 standard points (serially-diluted at 1:2 including a blank matrix sample). The brain lysate required an MRD of 50 with a dynamic range of 4.9-2500 ng/mL with a 10 standard point curve (serially-diluted at 1:2 plus a blank brain lysate sample. Serum and brain lysate sample concentrations were back-calculated off the assay-specific calibration standard curve, which was fitted with a weighed four-parameter non-linear logistic regression. The sample back-calculated concentrations in ng/mL were subsequently converted to nanomolar (nM) or micromolar (μM) as the final sample results.

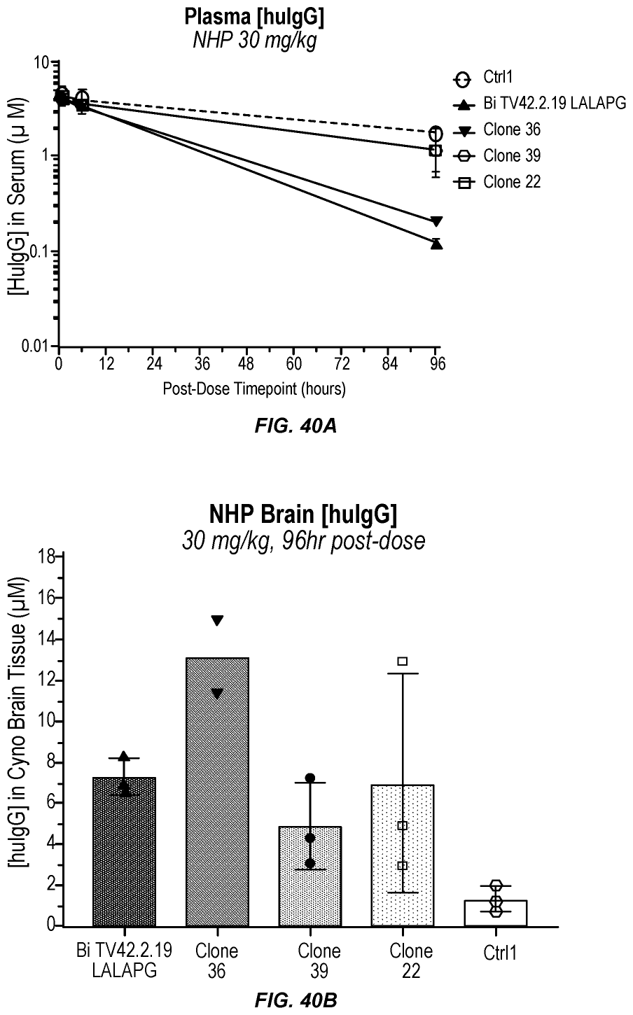

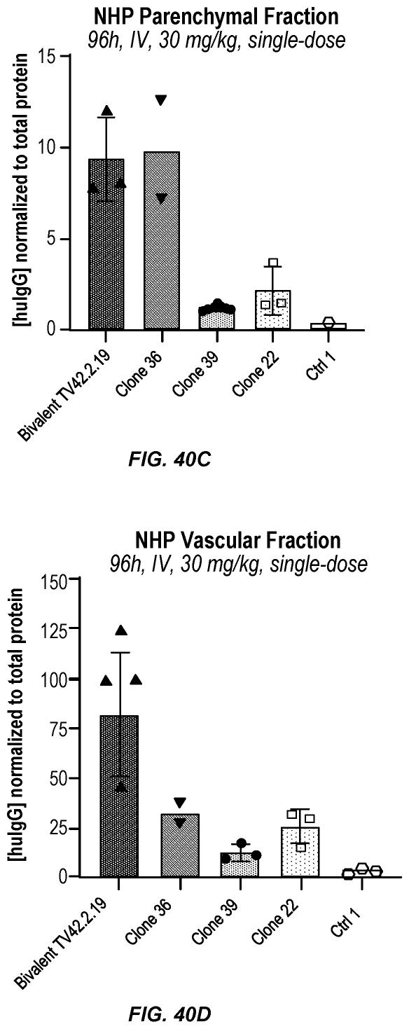

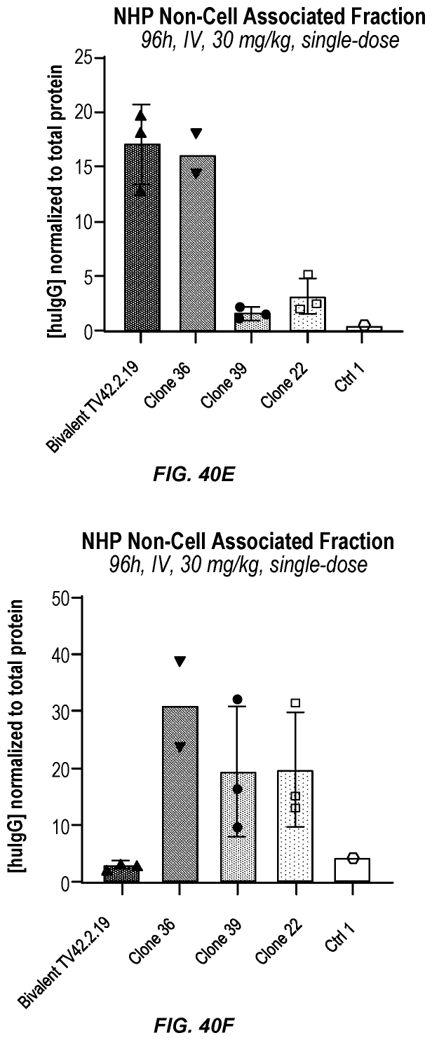

Results are shown in FIGS. 40A and 40B. Due to the expression of TfR and CD98hc on peripheral tissues, bivalent TV42 and LLB2 had faster clearance from serum compared to the non-binding control antibody. Consistent with effectively lower affinity CD98hc binding due lower valency and a lack of avidity, monovalent LLB2 molecules had intermediate serum clearance compared to the bivalent TVs and the control non-binding antibody. TV42 and all versions of LLB2 showed increased concentrations in brain, ranging from 5-13×, compared to a control non-binding antibody. One animal dosed with the bivalent LLB2 molecule was excluded from analysis due to signs of BBB disruption.

Capillary depletion was performed using the methods described above and huIgG concentration were measure in each fraction with the MSD described above. Results demonstrated uptake of LLB2 and TV42 into the NHP brain parenchyma (FIGS. 40C and 40D). TV42 was highly cell associated, bivalent LLB2 was found in both the cell associated and non-cell associated fractions, and monovalent LLB2s were highly non-cell associated (FIGS. 40E and 40F). The high cell association of TV42 is consistent with TfR being rapidly internalized. The increase in cell association for bivalent LLB2 compared to monovalent is consistent with mouse studies. For huIgG in the parenchymal, cell associated, and non-cell associated fractions, 2 of the control treated animals were below the lower limit of quantification of the assay.

Furthermore, in order to study any CNS cell type biodistribution differences in NHP, immunohistochemistry for huIgG and CNS cell type markers was performed on the NHP brain sections. After perfusion with PBS, the right hemisphere of the brain was collected and sectioned into 4 mm thick coronal slabs. The 4 mm thick slabs were placed into individual tissue cassettes and immersion fixed in 4% paraformaldehyde (PFA) and stored refrigerated (4 to 9° C.) for 48 hours. Immediately, following fixation, tissues were transferred to PBS+0.1% sodium azide. Thick slabs were further cut coronally into 40 μm sections on a microtome. Brain sections were blocked in 5% BSA+0.3% Triton X-100, followed by fluorescent staining with Alexa647 anti-huIgG (Southern Biotech 2049-31, 1:500) and rabbit anti-Iba1 (Abcam abl78846, 1:500)+goat anti-rabbit-568 (Invitrogen A-11011, 1:500). Additionally, sections were stained with Alexa647 anti-huIgG (Southern Biotech 2049-31, 1:500), rabbit anti-aquaporrin4 (Millipore AB2218, 1:500)+goat anti-rabbit-568 (Invitrogen A-11011, 1:500) and mouse anti-NeuN (Millipore MAB377, 1:500)+anti-msIgGl-488 (Invitrogen A21121, 1:500). Brain images were taken using a Leica SP8 Lightning confocal microscope with a 40× objective.

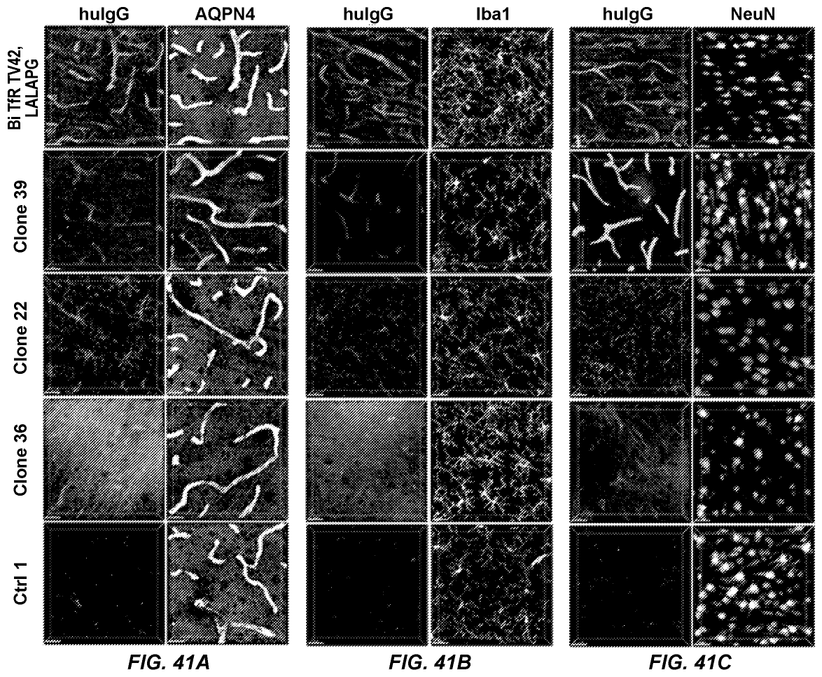

Results are shown in FIGS. 41A-41C. TV42 robustly colocalized with NeuN positive neurons (FIG. 41C). Monovalent LLB2 with WT effector function had a prominently microglial (Iba1) localization (FIG. 41B) whereas bivalent LLB2, regardless of effector function status, and monovalent LLB2 with LALAPG, i.e., effector negative, had a prominent colocalization with AQP4, consistent with localization to astrocytes (FIG. 41A). LLB2 variants were not observed to localize to neurons (FIG. 41C). LLB2 and TV42 had similar CNS cell type biodistribution in non-human primate as they do in mouse.

### Example 20. Design and Characterization of Engineered TFR-Binding Polypeptides from Naïve Phage Libraries

### Library Generation for Phage Display

A display phagemid was generated with a human Fc sequence fused to a 6×His tag, a c-Myc tag, and a truncated P3 protein from M13 phage. Mutagenic oligonucleotides containing degenerate codons at library positions were purchased from Integrated DNA Technologies. Using Künkel mutagenesis, a uridine-containing ssDNA template was generated and annealed with phosphorylated mutagenic oligonucleotides. T7 DNA polymerase and T4 DNA ligase was used to form a covalently closed circular dsDNA (CCC-dsDNA) library. The CCC-dsDNA library was electroporated into TG1 E. coli cells (Lucigen, 60502-2). Transformed cells were grown in SB media and infected with M13 K07 helper phage. Carbenicillin and kanamycin antibiotics were used to maintain the display and M13 K07 helper phagemids. The culture was grown overnight at 37° C. with shaking. Phage particles were precipitated from the media with a PEG/NaCl solution and resuspended in PBS.

The register was designed with diversity in residues that are predominantly located on the solvent-exposed side of the CH3 domain beta-sheet surface with additional residues adjacent to the beta-sheet. The library was generated by randomizing positions (according to EU numbering) 380, 382, 384, 385, 386, 387, 422, 424, 426, 438, and 440 on the Fc domain of hIgG1 with “NNK” degenerate codons. The library positions were mapped onto the structure of the Fc domain of human IgG1. The library was cloned onto a phagemid and displayed on phage with methods described above.

### Selection of TfR-Binding Clones by Phage Display

To select for human TfR-binding clones, recombinant biotinylated human TfR apical domain was captured by streptavidin magnetic beads (Invitrogen, 11206D). Human TfR-coated beads were washed and incubated with the phage library in PBS with 1% BSA for at least 1 hour at room temperature. Beads were washed 3 times for 1 minute each in PBS with 0.05% Tween-20. Bound phages were eluted with 100 mM glycine pH 2.7 for 15 minutes and neutralized with Tris-HCl buffer. Eluted phage was used to infect TG1 cells for additional rounds of selection. Four rounds of selection were performed. In each subsequent round, the concentration of soluble biotinylated human TfR apical domain was reduced and the time of washing was increased to provide selective binding pressure.

For libraries aimed at maturing binding affinity, phage libraries were incubated with 20 nM of soluble biotinylated human TfR apical domain for 30 minutes. Streptavidin magnetic beads were added for 5 minutes to capture biotinylated human TfR apical domain. Beads were washed 3 times for 15 minutes each in PBS with 0.05% Tween-20. Eluted phage was used to infect TG1 cells for additional rounds of selection. Four rounds of selection were performed. In each subsequent round, the time of washing was increased to provide selective binding pressure.

### Recombinant Expression of Clones

TfR-binding clones identified by phage display were reformatted by removing the 6×His tag, the c-Myc tag, and the truncated P3. A Fab was fused to the N-terminus of the clone to allow for surface plasmon resonance (SPR) capture (GE Healthcare, Anti-human Fab capture kit, 28958325). The fusions were expressed recombinantly in Expi293 cells and purified over Protein A.

### Measurement of Binding by Biacore

Binding affinities of Fab-clone fusions for TfR apical domain were determined by surface plasmon resonance using a Biacore™ 8K instrument in 1× HBS-EP+ running buffer (GE Healthcare, BR100669). Biacore™ Series S CM5 sensor chips were immobilized with anti-human Fab (GE Healthcare, 28958325). Molecules were captured on each flow cell and serial 3-fold dilutions of human TfR apical domain (2, 0.67, 0.22, 0.074, 0.025, and 0 μM) and cynomologous TfR apical domain (4, 1.3, 0.44, 0.15, 0.05, and 0 μM) were injected at a flow rate of 30 μL/min using single cycle kinetics method. Each sample was analyzed with a 60-second association and a 3-minute dissociation. After each cycle, the chip was regenerated using 10 mM glycine-HCl (pH 2.1) for 30 seconds at 50 μL/min. Binding response was corrected by subtracting the RU from a reference flow cell. A 1:1 Langmuir model of simultaneous fitting of kₒₙ and k_{off} was used for kinetics analysis using Biacore™ 8K Evaluation Software. Table 32A below summarizes the binding affinities of selected clones for human and cynomolgus TfR apical domains.

TABLE 32A

| | K_{D} for huTfR Apical | K_{D} for cyTfR |
|---|---|---|
| TfR-Binding Clone | Domain (nM) | Apical Domain (nM) |
| Clone 1 with LALA | 521 | 2010 |
| Clone 3 with LALA | 1450 | 6000 |
| Clone 1-15 with LALA and M428L | 260 | 2790 |
| Clone 1-34 with LALA and M428L | 383 | 3800 |
| Clone 1-40 with LALA and M428L | 253 | 1300 |
| Clone 1-41 with LALA and M428L | 366 | 1230 |
| Clone 1-44 with LALA and M428L | 455 | 2680 |
| Clone 1-50 with LALA and M428L | 138 | 1170 |
| Clone 1-55 with LALA and M428L | 93 | 609 |
| Clone 1-57 with LALA and M428L | 180 | 1160 |
| Clone 1-69 with LALA and M428L | 122 | 773 |
| Clone 1-70 with LALA and M428L | 83 | 520 |
| Clone 1-79 with LALA and M428L | 65 | 585 |
| Clone 1-84 with LALA and M428L | 123 | 859 |
| Clone 1-92 with LALA and M428L | 211 | 566 |
| Clone 1-105 with LALA and M428L | 507 | 968 |
| Clone 1-110 with LALA and M428L | 836 | 1300 |
| Clone 1-112 with LALA and M428L | 383 | 649 |
| Clone 1-117 with LALA and M428L | 189 | 1300 |
| Clone 1-120 with LALA and M428L | 118 | 698 |
| Clone 1-123 with LALA and M428L | 189 | 1330 |
| Clone 1-131 with LALA and M428L | 39 | 246 |
| Clone 1-133 with LALA and M428L | 99 | 752 |
| Clone 1-135 with LALA and M428L | 80 | 485 |
| Clone 1-142 with LALA and M428L | 751 | 4810 |
| Clone 1-143 with LALA and M428L | 104 | 687 |
| Clone 1-144 with LALA and M428L | 96 | 709 |

Cell uptake into TfR-positive cells was measured for clone 1 with LALA and clone 3 with LALA, showing uptake into human and cyno TfR-positive cells, but no uptake into negative control cells not expressing TfR.

### Initial Identification and Characterization of Clone 1 and Clone 3

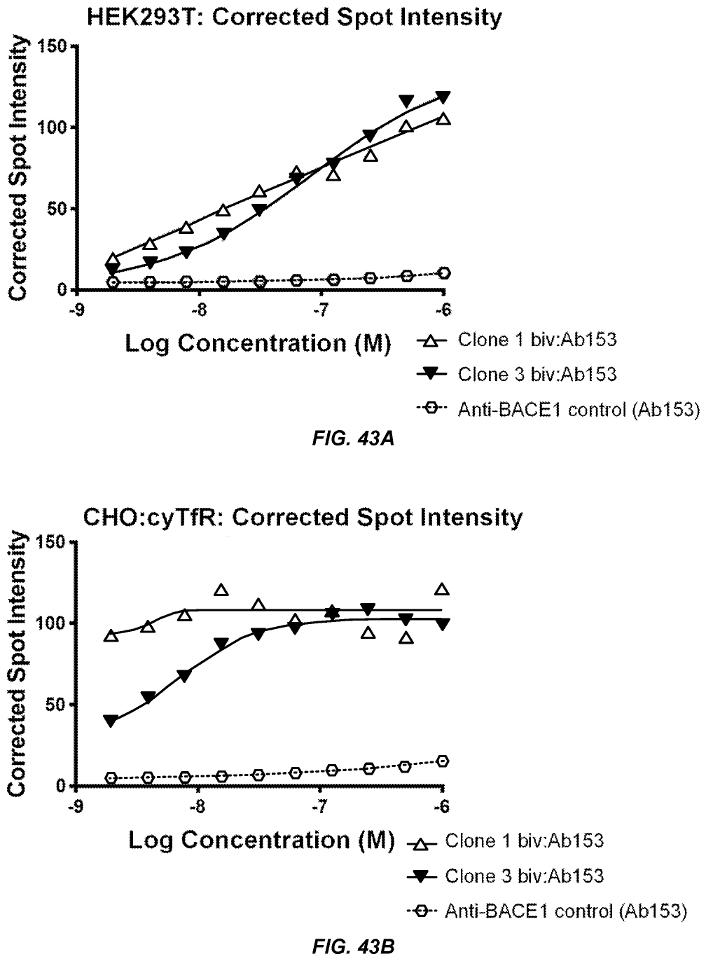

The phage library was panned against biotinylated human TfR apical domain immobilized to magnetic streptavidin beads. Four rounds of phage panning were performed and enriched clones were sequenced. Unique hits were cloned onto an anti-BACE1 hIgG1 monoclonal antibody containing the “LALA” mutations, expressed in HEK293 cells, and purified by protein A chromatography. Affinity to human and cyno TfR apical domain was measured by SPR. Clones were evaluated for TfR-specific cell binding (FIGS. 43A and 43B). Clone 1 and clone 3, each with LALA substitutions, have related sequences and were found to bind human and cyno TfR by SPR and on cells. Clone 1 with LALA binds human TfR apical domain with a K_{D} of 480 nM, cyno TfR apical domain with a K_{D} of 1800 nM, and had a clearance value of 7.6 mL/day/kg in WT mice. Clone 3 with LALA binds human TfR apical domain with a K_{D} of 1500 nM, cyno TfR apical domain with a K_{D} of 6000 nM, and had a clearance value of 20.6 mL/day/kg in WT mice.

### Affinity Maturation of Clone 1 by Soft Randomization

Clone 1 was selected for affinity maturation. Affinity maturation library AM1 was generated by soft randomizing positions 380, 382, 384, 385, 386, 387, 422, 424, 426, 438, and 440 of clone 1. Affinity maturation library AM2 was generated by keeping positions 382, 385, and 387 constant from clone 1 while hard randomizing positions 380, 386, 436, and 440 and soft randomizing positions 384, 422, 424, 426, 428, and 438. The libraries were cloned onto a phagemid and displayed on phage with methods described above. The AM1 and AM2 phage libraries were panned against biotinylated human and cyno TfR apical domain immobilized to magnetic streptavidin beads. Three rounds of phage panning were performed and enriched clones were sequenced. Periplasmic extracts of enriched clones were prepared and screened for binding by SPR. Top hits were cloned onto an anti-BACE1 hIgG1 monoclonal antibody containing the “LALA” mutations, expressed in HEK293 cells, and purified by protein A chromatography. Affinity to human and cyno TfR apical domain was measured by SPR (Table 32B). Several clones were selected and were shown to bind huTfR and cyTfR expressing cells. A multidose study was performed where 3 50 mg/kg IP doses of clone 1-112 with LALA and M428L were administered on day 0, 3, and 5 to chimeric huTfR^{apical} knock-in mice. On day 6, clone 1-112 with LALA and M428L showed significant uptake into the brain and demonstrated a pharmacodynamic response through BACE1 inhibition.

TABLE 32B

| | huTfR- | cyTfR- | |
|---|---|---|---|
| | Apical | Apical | Cy/Hu |
| Clone | K_{D} (nM) | K_{D} (nM) | K_{D} ratio |
| Clone 1 with LALA | 520 | 2000 | 3.8 |
| Clone 1-15 with LALA and M428L | 260 | 2800 | 10.8 |
| Clone 1-34 with LALA and M428L | 380 | 3800 | 10.0 |
| Clone 1-40 with LALA and M428L | 250 | 1300 | 5.2 |
| Clone 1-41 with LALA and M428L | 370 | 1200 | 3.2 |
| Clone 1-44 with LALA and M428L | 450 | 2700 | 6.0 |
| Clone 1-50 with LALA and M428L | 140 | 1200 | 8.6 |
| Clone 1-55 with LALA and M428L | 93 | 610 | 6.6 |
| Clone 1-57 with LALA and M428L | 180 | 1200 | 6.7 |
| Clone 1-69 with LALA and M428L | 120 | 770 | 6.4 |
| Clone 1-70 with LALA and M428L | 83 | 520 | 6.3 |
| Clone 1-79 with LALA and M428L | 65 | 590 | 9.1 |
| Clone 1-84 with LALA and M428L | 120 | 860 | 7.2 |
| Clone 1-92 with LALA and M428L | 210 | 570 | 2.7 |
| Clone 1-105 with LALA | 510 | 970 | 1.9 |
| and M428L | | | |
| Clone 1-110 with LALA | 840 | 1300 | 1.5 |
| and M428L | | | |
| Clone 1-112 with LALA | 380 | 650 | 1.7 |
| and M428L | | | |
| Clone 1-117 with LALA | 190 | 1300 | 6.8 |
| and M428L | | | |
| Clone 1-120 with LALA | 120 | 700 | 5.8 |
| and M428L | | | |
| Clone 1-123 with LALA | 190 | 1300 | 6.8 |
| and M428L | | | |
| Clone 1-131 with LALA | 39 | 250 | 6.4 |
| and M428L | | | |
| Clone 1-133 with LALA | 99 | 750 | 7.6 |
| and M428L | | | |
| Clone 1-135 with LALA | 80 | 490 | 6.1 |
| and M428L | | | |
| Clone 1-142 with LALA | 750 | 4800 | 6.4 |
| and M428L | | | |
| Clone 1-143 with LALA | 100 | 690 | 6.9 |
| and M428L | | | |
| Clone 1-144 with LALA | 96 | 710 | 7.4 |
| and M428L | | | |

Sequences of the clones shown in Tables 32A and 32B are shown in Table 32B-1 below. For the positions not listed, the amino acid at that position is the same as the one in the wild-type Fc.

TABLE 32B-1

| Clone | 380 | 381 | 382 | 383 | 384 | 385 | 386 | 422 | 423 | 424 | 425 | 426 | 436 | 437 | 438 | 439 | 440 |
|---|---|---|---|---|---|---|---|---|---|---|---|---|---|---|---|---|---|
| WT Fc | E | W | E | S | N | G | Q | V | F | S | C | S | Y | T | Q | K | S |
| Clone 1 | K | W | G | S | E | V | A | I | F | A | C | T | Y | T | L | K | M |
| Clone 3 | L | W | G | S | A | V | A | V | F | A | C | T | Y | T | V | K | G |
| Clone 1-15 | E | W | G | S | E | V | Q | I | F | A | C | T | Y | T | I | K | G |
| Clone 1-34 | I | W | G | S | E | V | A | I | F | A | C | T | F | T | V | K | A |
| Clone 1-40 | E | W | G | S | L | V | Q | H | F | A | C | T | Y | T | I | K | G |
| Clone 1-41 | Q | W | G | S | E | V | V | I | F | A | C | T | Y | T | I | K | T |
| Clone 1-44 | V | W | G | S | E | V | S | I | F | A | C | T | Y | T | V | K | G |
| Clone 1-50 | F | W | G | S | E | V | Q | I | F | A | C | T | Y | T | Y | K | G |
| Clone 1-55 | I | W | G | S | E | V | A | I | F | A | C | T | Y | T | L | K | M |
| Clone 1-57 | I | W | G | S | E | V | Q | I | F | A | C | T | Y | T | I | K | G |
| Clone 1-69 | M | W | G | S | A | V | S | V | F | A | C | T | Y | T | I | K | G |
| Clone 1-70 | M | W | G | S | E | V | A | I | F | A | C | T | Y | T | I | K | G |
| Clone 1-79 | M | W | G | S | E | V | M | R | F | P | C | I | F | T | I | K | V |
| Clone 1-84 | M | W | G | S | E | V | Q | L | F | A | C | T | F | T | Y | K | V |
| Clone 1-92 | Q | W | G | S | E | V | A | I | F | A | C | T | Y | T | L | K | M |
| Clone 1-105 | E | W | G | S | A | V | A | I | F | A | C | T | Y | T | I | K | G |
| Clone 1-110 | E | W | G | S | L | V | A | I | F | A | C | T | Y | T | V | K | G |
| Clone 1-112 | E | W | G | S | L | V | Q | I | F | A | C | T | Y | T | I | K | G |
| Clone 1-117 | L | W | G | S | E | V | Q | I | F | P | C | V | F | T | Y | K | A |
| Clone 1-120 | M | W | G | S | E | V | A | I | F | A | C | T | Y | T | I | K | A |
| Clone 1-123 | M | W | G | S | E | V | A | I | F | P | C | I | Y | T | I | K | G |
| Clone 1-131 | M | W | G | S | E | V | Q | I | F | P | C | I | Y | T | L | K | M |
| Clone 1-133 | M | W | G | S | E | V | S | R | F | P | C | I | Y | T | L | K | V |
| Clone 1-135 | M | W | G | S | L | V | Q | I | F | A | C | T | Y | T | I | K | T |
| Clone 1-142 | M | W | G | S | E | V | Q | R | F | P | C | I | Y | T | L | K | M |
| Clone 1-143 | M | W | G | S | E | V | Q | I | F | P | C | I | Y | T | Y | K | A |
| Clone 1-144 | M | W | G | S | E | V | Q | I | F | P | C | I | Y | T | I | K | G |

### Mutational Analysis of Clone 1-112

Clone 1-112 was selected for mutational analysis. To understand the relative impact of each position of the register on binding and human/cyno cross-reactivity, a panel of single, double, or triple rationally designed mutations were made to clone 1-112. These mutations included an alanine scan of the register, a reversion to WT Fe scan, conservative mutations, and mutations observed from sequencing affinity maturation libraries. Mutations were cloned onto 1-112 and expressed in HEK293 cells. Supernatants were captured for SPR by GE Healthcare anti-human Fab capture kit and the affinity to human and cyno TfR apical domain was measured (Table 32C and Table 32D). This analysis revealed mutations that are important for binding, affinity, and cross-reactivity for human and cyno TfR.

Table 32C and Table 32D below show the library of clone 1-112 mutants and their binding affinities. Each mutant contained one or more amino acid substitutions of clone 1-112. For example, one mutant may contain an E380A mutation and the amino acids at the rest of the positions are the same as those in clone 1-112. Further, to generate the K_{D} data in Table 32C, each of the clones in Table 32C (except for clones 1-112-102 to 1-112-104 and clones 1-112-106 to 1-112-114) also contained LALA and M428L. Clones 1-112-102 to 1-112-104 and clones 1-112-106 to 1-112-114 also contained LALA. The positions are numbered according to the EU numbering scheme.

TABLE 32C

| | | | | | | | | | | | | | | | | Ratio |
|---|---|---|---|---|---|---|---|---|---|---|---|---|---|---|---|---|
| | | | | | | | | | | | | | | | | of K_{D} |
| | | | | | | | | | | | | | | K_{D} for | K_{D} for | for |
| | | | | | | | | | | | | | | huTfR | cyTfR | cyTfR |
| | | | | | | | | | | | | | | Apical | Apical | to K_{D} |
| | | | | | | | | | | | | | | Domain | Domain | for |
| Clone | 378 | 380 | 382 | 384 | 385 | 386 | 421 | 422 | 424 | 426 | 438 | 440 | 442 | (nM) | (nM) | huTfR |
| Clone 1-112 | | E | G | L | V | | | I | A | T | I | G | | 380 | 670 | 1.8 |
| Clone 1-112-2 | | A | | | | | | | | | | | | 240 | 750 | 3.1 |
| Clone 1-112-3 | | D | | | | | | | | | | | | 220 | 440 | 2.0 |
| Clone 1-112-4 | | F | | | | | | | | | | | | 120 | 980 | 8.2 |
| Clone 1-112-5 | | H | | | | | | | | | | | | 400 | 2000 | 5.0 |
| Clone 1-112-6 | | I | | | | | | | | | | | | 230 | 1600 | 7.0 |
| Clone 1-112-7 | | K | | | | | | | | | | | | 240 | 1200 | 5.0 |
| Clone 1-112-8 | | L | | | | | | | | | | | | 130 | 910 | 7.0 |
| Clone 1-112-9 | | M | | | | | | | | | | | | 80 | 570 | 7.1 |
| Clone 1-112-10 | | Q | | | | | | | | | | | | 200 | 740 | 3.7 |
| Clone 1-112-11 | | | A | | | | | | | | | | | >4000 | >4000 | |
| Clone 1-112-12 | | | E | | | | | | | | | | | >4000 | >4000 | |
| Clone 1-112-13 | | | | A | | | | | | | | | | 750 | 1400 | 1.9 |
| Clone 1-112-14 | | | | D | | | | | | | | | | >4000 | >4000 | |
| Clone 1-112-15 | | | | E | | | | | | | | | | 1400 | 2200 | 1.6 |
| Clone 1-112-16 | | | | F | | | | | | | | | | 1400 | 3800 | 2.7 |
| Clone 1-112-17 | | | | G | | | | | | | | | | >4000 | >4000 | |
| Clone 1-112-18 | | | | H | | | | | | | | | | 4000 | >4000 | |
| Clone 1-112-19 | | | | I | | | | | | | | | | 1400 | 2400 | 1.7 |
| Clone 1-112-20 | | | | K | | | | | | | | | | 2200 | 3000 | 1.4 |
| Clone 1-112-21 | | | | N | | | | | | | | | | >4000 | >4000 | |
| Clone 1-112-22 | | | | P | | | | | | | | | | >4000 | >4000 | |
| Clone 1-112-23 | | | | Q | | | | | | | | | | 1100 | 2100 | 1.9 |
| Clone 1-112-24 | | | | S | | | | | | | | | | 3300 | >4000 | |
| Clone 1-112-25 | | | | T | | | | | | | | | | >4000 | >4000 | |
| Clone 1-112-26 | | | | V | | | | | | | | | | 1300 | 3600 | 2.8 |
| Clone 1-112-27 | | | | Y | | | | | | | | | | 2100 | >4000 | |
| Clone 1-112-28 | | | | | A | | | | | | | | | >4000 | >4000 | |
| Clone 1-112-29 | | | | | G | | | | | | | | | >4000 | >4000 | |
| Clone 1-112-30 | | | | | I | | | | | | | | | 3400 | >4000 | |
| Clone 1-112-31 | | | | | L | | | | | | | | | >4000 | >4000 | |
| Clone 1-112-32 | | | | | T | | | | | | | | | 2000 | 3600 | 1.8 |
| Clone 1-112-33 | | | | | | A | | | | | | | | 280 | 480 | 1.7 |
| Clone 1-112-34 | | | | | | G | | | | | | | | >4000 | >4000 | |
| Clone 1-112-35 | | | | | | H | | | | | | | | 470 | 950 | 2.0 |
| Clone 1-112-36 | | | | | | N | | | | | | | | 380 | 660 | 1.7 |
| Clone 1-112-37 | | | | | | S | | | | | | | | 400 | 730 | 1.8 |
| Clone 1-112-38 | | | | | | T | | | | | | | | 490 | 840 | 1.7 |
| Clone 1-112-39 | | | | | | | | A | | | | | | 3900 | >4000 | |
| Clone 1-112-40 | | | | | | | | H | | | | | | 3600 | >4000 | |
| Clone 1-112-41 | | | | | | | | K | | | | | | 2300 | >4000 | |
| Clone 1-112-42 | | | | | | | | L | | | | | | >4000 | >4000 | |
| Clone 1-112-43 | | | | | | | | R | | | | | | 1700 | 3300 | 1.9 |
| Clone 1-112-44 | | | | | | | | T | | | | | | 630 | 1200 | 1.9 |
| Clone 1-112-45 | | | | | | | | V | | | | | | 420 | 740 | 1.8 |
| Clone 1-112-46 | | | | | | | | Y | | | | | | 3000 | 5200 | 1.7 |
| Clone 1-112-47 | | | | | | | | | G | | | | | 2200 | 2700 | 1.2 |
| Clone 1-112-48 | | | | | | | | | H | | | | | >4000 | >4000 | |
| Clone 1-112-49 | | | | | | | | | P | | | | | 1100 | 1700 | 1.5 |
| Clone 1-112-50 | | | | | | | | | S | | | | | 700 | 1100 | 1.6 |
| Clone 1-112-51 | | | | | | | | | | A | | | | 2800 | >4000 | |
| Clone 1-112-52 | | | | | | | | | | G | | | | >4000 | >4000 | |
| Clone 1-112-53 | | | | | | | | | | H | | | | >4000 | >4000 | |
| Clone 1-112-54 | | | | | | | | | | I | | | | 560 | 1100 | 2.0 |
| Clone 1-112-55 | | | | | | | | | | L | | | | 930 | 2000 | 2.2 |
| Clone 1-112-56 | | | | | | | | | | S | | | | 1500 | 2000 | 1.3 |
| Clone 1-112-57 | | | | | | | | | | V | | | | 450 | 750 | 1.7 |
| Clone 1-112-61 | | | | | | | | | | | A | | | >4000 | >4000 | |
| Clone 1-112-62 | | | | | | | | | | | D | | | >4000 | >4000 | |
| Clone 1-112-63 | | | | | | | | | | | E | | | >4000 | >4000 | |
| Clone 1-112-64 | | | | | | | | | | | F | | | 2800 | 2300 | 0.8 |
| Clone 1-112-65 | | | | | | | | | | | G | | | >4000 | >4000 | |
| Clone 1-112-66 | | | | | | | | | | | H | | | >4000 | >4000 | |
| Clone 1-112-67 | | | | | | | | | | | K | | | >4000 | >4000 | |
| Clone 1-112-68 | | | | | | | | | | | L | | | 1200 | 2000 | 1.7 |
| Clone 1-112-69 | | | | | | | | | | | N | | | >4000 | >4000 | |
| Clone 1-112-70 | | | | | | | | | | | P | | | >4000 | >4000 | |
| Clone 1-112-71 | | | | | | | | | | | Q | | | >4000 | >4000 | |
| Clone 1-112-72 | | | | | | | | | | | S | | | >4000 | >4000 | |
| Clone 1-112-73 | | | | | | | | | | | T | | | >4000 | >4000 | |
| Clone 1-112-74 | | | | | | | | | | | V | | | 1200 | 1400 | 1.2 |
| Clone 1-112-75 | | | | | | | | | | | Y | | | 340 | 510 | 1.5 |
| Clone 1-112-76 | | | | | | | | | | | | A | | 360 | 530 | 1.5 |
| Clone 1-112-77 | | | | | | | | | | | | D | | >4000 | >4000 | |
| Clone 1-112-78 | | | | | | | | | | | | E | | >4000 | >4000 | |
| Clone 1-112-79 | | | | | | | | | | | | F | | >4000 | >4000 | |
| Clone 1-112-80 | | | | | | | | | | | | H | | >4000 | >4000 | |
| Clone 1-112-81 | | | | | | | | | | | | I | | 2400 | 1900 | 0.8 |
| Clone 1-112-82 | | | | | | | | | | | | K | | >4000 | >4000 | |
| Clone 1-112-83 | | | | | | | | | | | | L | | >4000 | >4000 | |
| Clone 1-112-84 | | | | | | | | | | | | M | | 340 | 400 | 1.2 |
| Clone 1-112-85 | | | | | | | | | | | | N | | 2100 | 2300 | 1.1 |
| Clone 1-112-86 | | | | | | | | | | | | P | | 3900 | 3600 | 0.9 |
| Clone 1-112-87 | | | | | | | | | | | | Q | | >4000 | >4000 | |
| Clone 1-112-88 | | | | | | | | | | | | S | | 610 | 810 | 1.3 |
| Clone 1-112-89 | | | | | | | | | | | | T | | 450 | 490 | 1.1 |
| Clone 1-112-90 | | | | | | | | | | | | V | | 550 | 820 | 1.5 |
| Clone 1-112-91 | | | | | | | | | | | | Y | | >4000 | >4000 | |
| Clone 1-112-92 | | | | | | | | | P | I | | | | 310 | 530 | 1.7 |
| Clone 1-112-93 | | | | E | | | | | P | | | | | >4000 | >4000 | |
| Clone 1-112-94 | | | | E | | | | | P | I | | | | 770 | 1300 | 1.7 |
| Clone 1-112-95 | | | | | | | | V | | | | S | | 2100 | 2300 | 1.1 |
| Clone 1-112-96 | | | | N | | | | V | | | | S | | >4000 | >4000 | |
| Clone 1-112-97 | Y | | | | | | | | | | | | | 180 | 890 | 4.9 |
| Clone 1-112-98 | | | | | | | L | | | | | | | 80 | 160 | 2.0 |
| Clone 1-112-99 | | | | | | | | | | | | | R | 50 | 90 | 1.8 |
| Clone 1-112-100 | Y | L | G | L | V | A | L | I | P | I | Y | A | R | 410 | 4900 | 12.0 |
| Clone 1-112-101 | | | G | L | V | A | L | I | P | I | Y | T | R | 180 | 430 | 2.4 |
| Clone 1-112-102 | | | G | L | V | | L | I | P | I | I | T | R | 41 | 52 | 1.3 |
| Clone 1-112-103 | | | G | L | V | | L | I | P | I | Y | T | R | 57 | 83 | 1.5 |
| Clone 1-112-104 | | | G | L | V | | L | I | A | T | Y | T | R | 390 | 580 | 1.5 |
| Clone 1-112-106 | | | G | L | V | | | | | | Y | | R | >10000 | >10000 | |
| Clone 1-112-107 | | | | L | V | | L | | | | Y | | R | >10000 | >10000 | |
| Clone 1-112-108 | | | G | L | V | | | | | T | Y | | R | 6100 | 9300 | 1.5 |
| Clone 1-112-109 | | | | L | V | | L | | | T | Y | | R | 1900 | 3000 | 1.6 |
| Clone 1-112-110 | | | | L | V | | | | A | T | Y | | R | 4300 | 5700 | 1.3 |
| Clone 1-112-111 | | | G | L | V | | L | | A | T | Y | | | >10000 | >10000 | |
| Clone 1-112-112 | | | | L | V | | L | | A | T | Y | | R | 1200 | 1900 | 1.6 |
| Clone 1-112-113 | | | G | L | V | | | I | A | T | Y | A | | 2700 | 4200 | 1.6 |
| Clone 1-112-114 | | | G | L | V | | | I | P | I | Y | A | | 1200 | 1800 | 1.5 |

TABLE 32D

| | K_{D} for | K_{D} for | Ratio of |
|---|---|---|---|
| | huTfR | cyTfR | K_{D} for |
| | Apical | Apical | cyTfR to |
| | Domain | Domain | K_{D} for |
| Clone | (nM) | (nM) | huTfR |
| Clone 1-112 with LALA and M428A | 1300 | 2100 | 1.6 |
| Clone 1-112 with LALA | 1300 | 1500 | 1.2 |
| Clone 1-112 with LALA, M428L, and N343S | 880 | 1400 | 1.6 |
| Clone 1-112-103 with LALA, M428L, and N434S | 31 | 77 | 2.5 |

### Second Round of Affinity Maturation of Clone 1 Family

For the second round of affinity maturation, three additional phage libraries were generated. Affinity maturation library AM3 was generated with degenerate or non-degenerate codons at the specified positions: “VWR” at 380, “GGG” at 382, “STIR” at 384, “GTG” at 385, “KCN” or “CAG” at 386, “ADA” at 422, “BCN” at 424, “RBY” at 426, “CTG” at 428, “ARY” at 434, “VTH” at 438, and “RBB” at 440. Affinity maturation library AM4 was generated with degenerate or non-degenerate codons at the specified positions: “NNK” at 378, “VWR” at 380, “GGG” at 382, “GAG” at 384, “GTG” at 385, “GCC” or “CAG” at 386, “NHIK” at 389, “NHIK” at 391, “NHIK” at 421, “ADA” at 422, “BCN” at 424, “RBY” at 426, “CTG” at 428, “ARY” at 434, “VTH” at 438, “RBB” at 440, and “NNK” at 442. Affinity maturation library AM5 was generated with degenerate or non-degenerate codons at the specified positions: “VWR” at 380, “NNK” at 382, “NNK” at 383, “NNK” at 384, “NNK” at 385, “NNK” at 386, “ADA” at 422, “BCN” at 424, “RBY” at 426, “CTG” at 428, “ARY” at 434, “VTH” at 438, and “RBB” at 440. The libraries were cloned onto a phagemid and displayed on phage with methods described above. Three rounds of solution phage panning was performed with increasing stringency per round and enriched clones were sequenced. Periplasmic extracts of enriched clones were prepared and screened for binding by SPR. Top hits were cloned onto an anti-BACE1 hIgG1 monoclonal antibody containing “LALA” mutations.

### Additional Engineering of Clone 1-131

Additional clones were designed by incorporating enriched mutations from the second round of affinity maturation of clone 1 onto clone 1-131. These clones were expressed in HEK293 cells and purified by protein A chromatography. Affinity to human and cyno TfR apical domain was measured by SPR.

Table 32E below shows the library of affinity matured clones and their binding affinities. Each mutant contained several amino acid substitutions relative to wild-type Fc. For the empty cells in Table 32E, the amino acid at that position is the same as the one in the wild-type Fc. Further, to generate the K_{D} data in Table 32E, each of the clones in Table 32E also contained LALA and M428L. The positions are numbered according to the EU numbering scheme.

TABLE 32E

| Clone | 378 | 380 | 382 | 383 | 384 | 385 | 386 | 389 | 391 | 421 | 422 |
|---|---|---|---|---|---|---|---|---|---|---|---|
| WT Fc | A | E | E | S | N | G | Q | N | Y | N | V |
| Clone 1-131 | | M | G | | E | V | | | | | I |
| Clone 1-131-3 | Y | M | G | | E | V | | | | | I |
| Clone 1-131-4 | | M | G | | E | V | | | | Q | I |
| Clone 1-131-5 | | M | G | | E | V | | | | | I |
| Clone 1-131-6 | Y | M | G | | E | V | | | | | I |
| Clone 1-168 | | L | G | | E | V | | | | L | R |
| Clone 1-174 | F | M | G | | E | V | | H | | L | I |
| Clone 1-185 | H | M | G | | E | V | | F | | L | R |
| Clone 1-188 | H | M | G | | E | V | | T | | L | I |
| Clone 1-194 | S | M | G | | E | V | | T | | L | I |
| Clone 1-195 | Y | | G | | E | V | | | | L | R |
| Clone 1-199 | Y | M | G | | E | V | | | | Q | I |
| Clone 1-200 | | | G | | E | V | | | | L | R |
| Clone 1-201 | | | G | | E | V | | S | | L | I |
| Clone 1-209 | D | M | G | | E | V | | T | | K | I |
| Clone 1-210 | H | | G | | E | V | | S | | L | R |
| Clone 1-211 | H | | G | | E | V | | V | | K | I |
| Clone 1-212 | H | M | G | | E | V | | Y | | L | I |
| Clone 1-232 | | | G | T | L | V | | | | | R |
| Clone 1-239 | H | M | G | | E | V | | F | | Y | I |

| | | | | | | | | Ratio |
|---|---|---|---|---|---|---|---|---|
| | | | | | | K_{D} | K_{D} | of K_{D} |
| | | | | | | for | for | for |
| | | | | | | huTfR | cyTfR | cyTfR |
| | | | | | | Apical | Apical | to K_{D} |
| | | | | | | Domain | Domain | for |
| Clone | 424 | 426 | 438 | 440 | 442 | (nM) | (nM) | huTfR |
| WT Fc | S | S | Q | S | S | | | |
| Clone 1-131 | P | I | L | M | | 34.0 | 212 | 6.2 |
| Clone 1-131-3 | P | I | L | M | | 29.1 | 350 | 12.0 |
| Clone 1-131-4 | P | I | L | M | | 12.2 | 78 | 6.4 |
| Clone 1-131-5 | P | I | L | M | R | 4.3 | 24 | 5.5 |
| Clone 1-131-6 | P | I | L | M | R | 3.3 | 36 | 10.9 |
| Clone 1-168 | P | I | Y | V | T | 3.9 | 38 | 9.7 |
| Clone 1-174 | P | I | I | G | R | 3.1 | 33 | 10.9 |
| Clone 1-185 | P | I | Y | V | A | 2.7 | 29 | 10.8 |
| Clone 1-188 | P | I | V | G | R | 2.5 | 27 | 10.7 |
| Clone 1-194 | P | I | I | A | R | 3.6 | 18 | 5.0 |
| Clone 1-195 | P | I | Y | V | T | 17.8 | 75 | 4.2 |
| Clone 1-199 | P | I | L | M | R | 1.2 | 14 | 11.2 |
| Clone 1-200 | P | I | I | V | R | 56.3 | 84 | 1.5 |
| Clone 1-201 | P | I | Y | A | R | 18.6 | 26 | 1.4 |
| Clone 1-209 | P | I | Y | G | R | 9.5 | 38 | 4.0 |
| Clone 1-210 | P | I | Y | V | | 28.6 | 73 | 2.5 |
| Clone 1-211 | P | I | I | G | R | | | |
| Clone 1-212 | P | I | Y | | R | 1.5 | 15 | 10.0 |
| Clone 1-232 | A | V | Y | V | | 51.7 | 90 | 1.7 |
| Clone 1-239 | P | I | Y | A | R | 1.9 | 19 | 9.8 |

### Additional Engineering of Clone 1-112

Mutations N421L, Q438Y, and S442R were found to improve the affinity to human and cyno TfR apical domain by SPR. These mutations were incorporated onto clone 1-112 in order to broaden the affinity range. These mutations were also combined with WT Fc reversion mutations in order to weaken the affinity and reduce the total number of mutations. Variants were cloned as a monovalent dimer onto the heavy chain of an anti-BACE hIgG1 monoclonal antibody containing “LALA” and knob mutations. The clones were expressed in EK293 cells and purified by protein A chromatography. Affinity to human and cyno TfR apical domain was measured by SPR (Table 32F). Molecules containing N421L, Q438Y, or S442R mutations displayed weaker cell binding than anticipated. The affinity for full-length human TfR was measured by SPR for a select set of molecules. Clone 1-112 had a similar binding affinity for full-length human TfR and the apical domain ofhuman TfR. Molecules containingN421L, Q438Y, or S442 Rmutations bound to full-length human TfR with a weaker affinity than to the apical domain of human TfR. It was concluded that the affinity improvements observed from these mutations were specific to the apical domain construct.

TABLE 32F

| | huTfR- | cyTfR- | Cy/Hu | huTfR- |
|---|---|---|---|---|
| | Apical K_{D} | Apical K_{D} | K_{D} | Full-Length |
| Clone | (nM) | (nM) | ratio | K_{D} (nM) |
| Clone 1-112 with LALA and M428L | 480 | 1100 | 2.3 | 470 |
| Clone 1-112-100 with LALA and M428L | 410 | 4900 | 12.0 | N/A |
| Clone 1-112-101 with LALA and M427L | 180 | 430 | 2.4 | N/A |
| Clone 1-112-102 with LALA | 41 | 52 | 1.3 | 580 |
| Clone 1-112-103 with LALA | 57 | 83 | 1.5 | 1500 |
| Clone 1-112-103 with LALA, M428L, and N434S | 31 | 77 | 2.5 | 880 |
| Clone 1-112-104 with LALA | 390 | 580 | 1.5 | 4200 |
| Clone 1-112-106 with LALA | >10000 | >10000 | | N/A |
| Clone 1-112-107 with LALA | >10000 | >10000 | | N/A |
| Clone 1-112-108 with LALA | 6100 | 9300 | 1.5 | N/A |
| Clone 1-112-109 with LALA | 1900 | 3000 | 1.6 | N/A |
| Clone 1-112-110 with LALA | 4300 | 5700 | 1.3 | N/A |
| Clone 1-112-111 with LALA | >10000 | >10000 | | N/A |
| Clone 1-112-112 with LALA | 1200 | 1900 | 1.6 | >10000 |
| Clone 1-112-113 with LALA | 2700 | 4200 | 1.6 | 8700 |
| Clone 1-112-114 with LALA | 1200 | 1800 | 1.5 | 9300 |

To broaden the affinity of clone 1-112, an additional panel of variants were generated by combining mutations from the mutational analysis study. Engineered molecules in this set contained an N434S mutation. Variants were cloned as a monovalent dimer onto the heavy chain of an anti-BACE1 hIgG1 monoclonal antibody containing “LALA” and knob mutations. The second anti-BACE1 heavy chain contained “LALA”, hole, and “LS” mutations. The clones were expressed in HEK293 cells and purified by protein A chromatography. Affinity to human and cyno TfR apical domain was measured by SPR (Table 32G).

TABLE 32G

| | huTfR-Apical | cyTfR-Apical | Cy/Hu |
|---|---|---|---|
| Clone | K_{D} (nM) | K_{D} (nM) | K_{D} ratio |
| 1-112 with LALA and M428L | 440 | 920 | 2.1 |
| 1-112 with LALA, M428L, and N434S | 1100 | 2700 | 2.5 |
| 1-112-2 with LALA, M428L, and N434S | 870 | 6600 | 7.6 |
| 1-112-3 with LALA, M428L, and N434S | 670 | 2400 | 3.6 |
| 1-112-4 with LALA, M428L, and N434S | 470 | 7000 | 14.9 |
| 1-112-8 with LALA, M428L, and N434S | 470 | 7500 | 16.0 |
| 1-112-10 with LALA, M428L, and N434S | 650 | 3300 | 5.1 |
| 1-112-15 with LALA, M428L, and N434S | 2300 | 5500 | 2.4 |
| 1-112-122 with with LALA, M428L, | 590 | 3300 | 5.6 |
| and N434S | | | |
| 1-112-45 with LALA, M428L, and N434S | 950 | 2600 | 2.7 |
| 1-112-50 with LALA, M428L, and N434S | 1500 | 4600 | 3.1 |
| 1-112-75 with LALA, M428L, and N434S | 700 | 1600 | 2.3 |
| 1-112-88 with LALA, M428L, and N434S | 1100 | 2900 | 2.6 |
| 1-112-89 with LALA, M428L, and N434S | 700 | 1600 | 2.3 |
| 1-112-92 with LALA, M428L, and N434S | 730 | 1900 | 2.6 |

### Third Round of Affinity Maturation of Clone 1 Family

For the third round of affinity maturation, affinity maturation library AM6 was generated with degenerate or non-degenerate codons at the specified positions: “NHW” at 378, “NHW” at 380, “GGC” at 382, “VHIW” at 384, “GTG” at 385, “NHIW” at 386, “NTY” at 422, “BCN” at 424, “ABY” at 426, “CTG” at 428, “TCC” at 434, “NoeW” at 438, and “NNW” at 440. Three rounds of solution phage panning was performed with increasing stringency per round and enriched clones were sequenced. Periplasmic extracts of enriched clones were prepared and screened for binding by SPR. Top hits were cloned as a monovalent dimer onto the heavy chain of an anti-BACE hIgG1 monoclonal antibody containing “LALA” and knob mutations. The second anti-BACE1 heavy chain contained “LALA” and hole mutations. “LS” mutations were included on the “hole” heavy chain for clone 1-244 to clone 1-334. These clones were expressed in HEK293 cells and purified by protein A chromatography. Affinity to human and cyno TfR apical domain was measured by SPR (Table 32H). All residues and mutations observed in a validated TfR binder are summarized in Table 32I.

TABLE 32H

| | huTfR-Apical | cyTfR-Apical | Cy/Hu |
|---|---|---|---|
| Clone | K_{D} (nM) | K_{D} (nM) | K_{D} ratio |
| Clone 1-112 with LALA, M428L | 440 | 920 | 2.1 |
| Clone 1-244 with LALA, M428L, and N434S | 170 | 1600 | 9.4 |
| Clone 1-252 with LALA, M428L, and N434S | 340 | 710 | 2.1 |
| Clone 1-265 with LALA, M428L, and N434S | 71 | 1300 | 18.3 |
| Clone 1-285 with LALA, M428L, and N434S | 360 | 750 | 2.1 |
| Clone 1-292 with LALA, M428L, and N434S | 280 | 1200 | 4.3 |
| Clone 1-300 with LALA, M428L, and N434S | 150 | 1800 | 12.0 |
| Clone 1-303 with LALA, M428L, and N434S | 410 | 1200 | 2.9 |
| Clone 1-317 with LALA, M428L, and N434S | 670 | 1200 | 1.8 |
| Clone 1-320 with LALA, M428L, and N434S | 97 | 2000 | 20.6 |
| Clone 1-321 with LALA, M428L, and N434S | 210 | 720 | 3.4 |
| Clone 1-331 with LALA, M428L, and N434S | 430 | 1000 | 2.3 |
| Clone 1-334 with LALA, M428L, and N434S | 180 | 900 | 5.0 |
| Clone 1-265 with LALA and M428L | 37 | 660 | 17.8 |
| Clone 1-292 with LALA and M428L | 130 | 480 | 3.7 |
| Clone 1-320 with LALA and M428L | 57 | 1100 | 19.3 |
| Clone 1-321 with LALA and M428L | 95 | 390 | 4.1 |

Sequences of the clones shown in Table 32H are shown in Table 32H-1 below. For the positions not listed, the amino acid at that position is the same as the one in the wild-type Fc.

TABLE 32H-1

| Clone | 378 | 379 | 380 | 381 | 382 | 383 | 384 | 385 | 386 | 422 | 423 | 424 | 425 | 426 | 438 | 439 | 440 |
|---|---|---|---|---|---|---|---|---|---|---|---|---|---|---|---|---|---|
| WT Fc | A | V | E | W | E | S | N | G | Q | V | F | S | C | S | Q | K | S |
| Clone 1-244 | D | V | F | W | G | S | A | V | Q | F | F | P | C | I | I | K | V |
| Clone 1-252 | Y | V | Y | W | G | S | L | V | Q | L | F | P | C | I | Y | K | V |
| Clone 1-265 | H | V | F | W | G | S | L | V | Q | F | F | P | C | I | Y | K | V |
| Clone 1-285 | S | V | E | W | G | S | L | V | Q | F | F | P | C | I | Y | K | V |
| Clone 1-292 | A | V | D | W | G | S | L | V | A | V | F | A | C | I | I | K | G |
| Clone 1-300 | V | V | F | W | G | S | E | V | Q | F | F | P | C | I | Y | K | V |
| Clone 1-303 | A | V | D | W | G | S | L | V | A | I | F | A | C | I | I | K | T |
| Clone 1-317 | D | V | E | W | G | S | L | V | A | F | F | P | C | I | I | K | V |
| Clone 1-320 | F | V | F | W | G | S | E | V | Q | L | F | P | C | I | Y | K | V |
| Clone 1-321 | H | V | E | W | G | S | L | V | Q | I | F | P | C | I | I | K | T |
| Clone 1-331 | S | V | E | W | G | S | L | V | S | F | F | P | C | I | I | K | V |
| Clone 1-334 | Y | V | E | W | G | S | L | V | Q | I | F | P | C | I | I | K | T |

### PK/PD Evaluation of Clone 1 and Clone 3

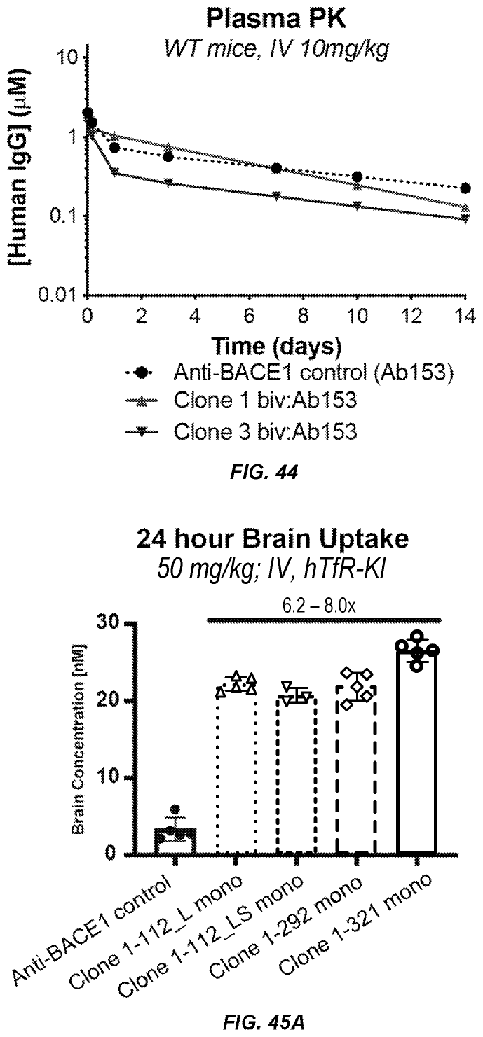

Fe fragments containing clone 1 and clone 3 each fused to an anti-BACEl Fab (21H₈) were tested for their PK in wild-type TfR mice to ensure that there are no PK liabilities. “Clone 1 biv:Ab 153” is a bivalent Fc-Fab fusion polypeptide comprising two Fc polypeptides each comprising the sequence of clone 1 with LALA and M428L, fused to the high affinity anti-BACEl Fab domain (Ab 153). “Clone 3 biv:Ab 153” is is a bivalent Fc-Fab fusion polypeptide comprising two Fc polypeptides each comprising the sequence of clone 3 with LALA and M428L, fused to the high affinity anti-BACEl Fab domain (Ab153). “Anti-BACEl control” is a negative control without any TfR-binding site. Clone 1 had normal clearance relative to the negative control (FIG. 44). The clearance values for clones 1 and 3 are 7.6 mL/day/kg and 20.6 mL/day/kg, respectively, and the clearance values for the negative control is 6.3 mL/day/kg.

From analysis of the TfR-binding clones, Table 32I further summarizes possible amino acids at each of the positions that has led to a TfR binder. The positions are numbered according to the EU numbering scheme.

TABLE 32I

| | Position | | | | | | | | | | | | | | | | |
|---|---|---|---|---|---|---|---|---|---|---|---|---|---|---|---|---|---|
| | 378 | 380 | 382 | 383 | 384 | 385 | 386 | 387 | 421 | 422 | 424 | 426 | 428 | 434 | 438 | 440 | 442 |
| WT Fc | A | E | E | S | N | G | Q | P | N | V | S | S | M | N | Q | S | S |
| Possible | A | A | G | S | A | I | A | P | A | A | A | A | A | N | I | A | A |
| Amino | D | D | | T | E | T | H | | F | F | G | I | M | S | F | G | K |
| Acids at | E | E | | | F | V | N | | H | H | P | L | L | | L | I | M |
| Each | F | F | | | H | | Q | | K | I | S | S | | | V | M | R |
| Position | H | H | | | I | | S | | L | K | | T | | | Y | N | S |
| | N | I | | | K | | T | | M | L | | V | | | | P | T |
| | Q | K | | | L | | V | | N | R | | | | | | S | V |
| | S | L | | | Q | | | | Q | T | | | | | | T | |
| | V | M | | | S | | | | S | V | | | | | | V | |
| | Y | Q | | | V | | | | T | Y | | | | | | | |
| | | S | | | Y | | | | V | | | | | | | | |
| | | T | | | | | | | Y | | | | | | | | |
| | | Y | | | | | | | | | | | | | | | |

Additional Clones from Rational Design and Affinity Maturation

To further broaden the affinity of clone 1-112, an additional panel of variants were generated by combining mutations from the mutational analysis study. Each mutant in Table 32J contained several amino acid substitutions relative to wild-type Fc. For the empty cells in Table 32J, the amino acid at that position is the same as the one in the wild-type Fc. The clones were expressed recombinantly and tested for human TfR apical domain binding by Biacore™ with an estimated affinity around 57-2300 nM, with very weak cross-reactivity to cyno TfR apical domain (Table 32K).

TABLE 32J

| Clone | 378 | 380 | 382 | 384 | 385 | 386 | 422 | 424 | 426 | 438 | 440 |
|---|---|---|---|---|---|---|---|---|---|---|---|
| WT Fc | A | | E | N | G | Q | V | S | S | Q | S |
| 1-112-115 | | A | G | L | V | | I | A | T | I | G |
| 1-112-116 | | D | G | L | V | | I | A | T | I | G |
| 1-112-117 | | F | G | L | V | | I | A | T | I | G |
| 1-112-119 | | L | G | L | V | | I | A | T | I | G |
| 1-112-120 | | Q | G | L | V | | I | A | T | I | G |
| 1-112-121 | | | G | E | V | | I | A | T | I | G |
| 1-112-122 | | K | G | E | V | | I | A | T | I | G |
| 1-112-123 | | | G | L | V | | | A | T | I | G |
| 1-112-124 | | | G | L | V | | I | | T | I | G |
| 1-112-125 | | | G | L | V | | I | A | T | Y | G |
| 1-112-126 | | | G | L | V | | I | A | T | I | |
| 1-112-127 | | | G | L | V | | I | A | T | I | T |
| 1-112-128 | | | G | L | V | | I | P | I | I | G |

TABLE 32K

| | | | K_{D} for | K_{D} for |
|---|---|---|---|---|
| | | | huTfR | cyTfR |
| | | | Apical | Apical |
| | | | Domain | Domain |
| Fc Dimer | First Fc Polypeptide | Second Fc Polypeptide | (nM) | (nM) |
| 1-112-115 | 1-112-115 TfR-binding site | Fc sequence with T366S, | 870 | 6600 |
| | with T366W knob, LALA, | L368A, and Y407V hole, | | |
| | M428L, and N434S mutations | M428L, N434S, and LALA | | |
| | | mutations | | |
| 1-112-116 | 1-112-116 TfR-binding site | Fc sequence with T366S, | 670 | 2400 |
| | with T366W knob, LALA, | L368A, and Y407V hole, | | |
| | M428L, and N434S mutations | M428L, N434S, and LALA | | |
| | | mutations | | |
| 1-112-117 | 1-112-117 TfR-binding site | Fc sequence with T366S, | 470 | 7000 |
| | with T366W knob, LALA, | L368A, and Y407V hole, | | |
| | M428L, and N434S mutations | M428L, N434S, and LALA | | |
| | | mutations | | |
| 1-112-119 | 1-112-119 TfR-binding site | Fc sequence with T366S, | 470 | 7500 |
| | with T366W knob, LALA, | L368A, and Y407V hole, | | |
| | M428L, and N434S mutations | M428L, N434S, and LALA | | |
| | | mutations | | |
| 1-112-120 | 1-112-120 TfR-binding site | Fc sequence with T366S, | 650 | 3300 |
| | with T366W knob, LALA, | L368A, and Y407V hole, | | |
| | M428L, and N434S mutations | M428L, N434S, and LALA | | |
| | | mutations | | |
| 1-112-121 | 1-112-121 TfR-binding site | Fc sequence with T366S, | 2300 | 5500 |
| | with T366W knob, LALA, | L368A, and Y407V hole, | | |
| | M428L, and N434S mutations | M428L, N434S, and LALA | | |
| | | mutations | | |
| 1-112-122 | 1-112-122 TfR-binding site | Fc sequence with T366S, | 590 | 3300 |
| | with T366W knob, LALA, | L368A, and Y407V hole, | | |
| | M428L, and N434S mutations | M428L, N434S, and LALA | | |
| | | mutations | | |
| 1-112-123 | 1-112-123 TfR-binding site | Fc sequence with T366S, | 950 | 2600 |
| | with T366W knob, LALA, | L368A, and Y407V hole, | | |
| | M428L, and N434S mutations | M428L, N434S, and LALA | | |
| | | mutations | | |
| 1-112-124 | 1-112-124 TfR-binding site | Fc sequence with T366S, | 1500 | 4600 |
| | with T366W knob, LALA, | L368A, and Y407V hole, | | |
| | M428L, and N434S mutations | M428L, N434S, and LALA | | |
| | | mutations | | |
| 1-112-125 | 1-112-125 TfR-binding site | Fc sequence with T366S, | 700 | 1600 |
| | with T366W knob, LALA, | L368A, and Y407V hole, | | |
| | M428L, and N434S mutations | M428L, N434S, and LALA | | |
| | | mutations | | |
| 1-112-126 | 1-112-126 TfR-binding site | Fc sequence with T366S, | 1100 | 2900 |
| | with T366W knob, LALA, | L368A, and Y407V hole, | | |
| | M428L, and N434S mutations | M428L, N434S, and LALA | | |
| | | mutations | | |
| 1-112-127 | 1-112-127 TfR-binding site | Fc sequence with T366S, | 700 | 1600 |
| | with T366W knob, LALA, | L368A, and Y407V hole, | | |
| | M428L, and N434S mutations | M428L, N434S, and LALA | | |
| | | mutations | | |
| 1-112-128 | 1-112-128 TfR-binding site | Fc sequence with T366S, | 730 | 1900 |
| | with T366W knob, LALA, | L368A, and Y407V hole, | | |
| | M428L, and N434S mutations | M428L, N434S, and LALA | | |
| | | mutations | | |

### PK/PD Evaluation of Clones 1-112_L, 1-112_LS, 1-292, and 1-321

Clones 1-112_L monovalent, 1-112_LS monovalent, 1-292 monovalent, and 1-321 monovalent were tested for their PK in wild-type TfR mice to ensure that there are no PK liabilities. Clone 1-112_L monovalent used here contained 1-112 TfR-binding site with T366W knob, M428L, and LALA mutations as the first Fc polypeptide and Fc sequence with T366S, L368A, and Y407V hole, M428L, and LALA mutations as the second Fc polypeptide. Clone 1-112_LS monovalent used here contained 1-112 TfR-binding site with T366W knob, M428L, N434S, and LALA mutations as the first Fc polypeptide and Fc sequence with T366S, L368A, and Y407V hole, M428L, N434S, and LALA mutations as the second Fc polypeptide. Clone 1-292 monovalent used here contained 1-292 TfR-binding site with T366W knob, M428L, and LALA mutations as the first Fc polypeptide and Fc sequence with T366S, L368A, and Y407V hole, M428L, and LALA mutations as the second Fc polypeptide. Clone 1-321 monovalent used here contained 1-321 TfR-binding site with T366W knob, M428L, and LALA mutations as the first Fc polypeptide and Fc sequence with T366S, L368A, and Y407V hole, M428L, and LALA mutations as the second Fc polypeptide. Anti-BACE1 control is a negative control without any TfR-binding site.

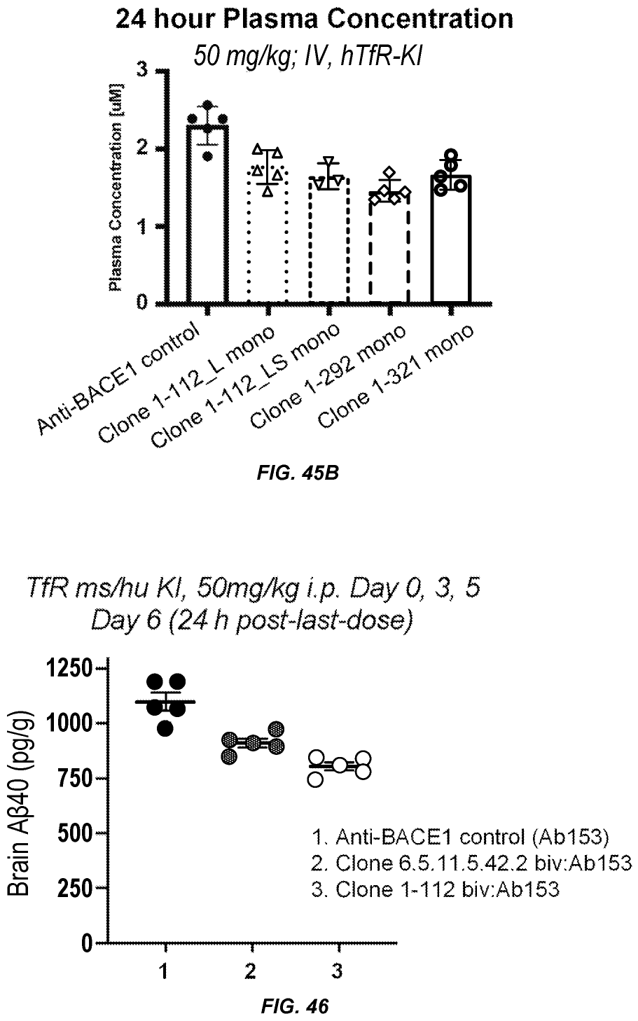

The clones and the controls were dosed intravenously into huTfR^{apical} knock-in mice at 50 mg/kg. Brain and plasma concentrations were measured at 24 hours (FIGS. 45A and 45B). All clones showed good brain uptake at 24 hours post-dose compared to the negative control (FIG. 45A). Plasma PK showed a clearance of all TfR-binding clones compared to the control as expected (FIG. 45B).

### Example 21. PK/PD Evaluation of Clone 6.5.11.5.42 and Clone 1-112

In order to determine if the clones were safe after multiple doses, a multidose safety study was conducted using clone 6.5.11.5.42.2 and clone 1-112. “Clone 6.5.11.5.42.2 biv:Ab153” is a bivalent Fc-Fab fusion polypeptide comprising two Fc polypeptides each comprising the sequence of clone 6.5.11.5.42.2, fused to the high affinity anti-BACE1 Fab domain (Ab153); “clone 1-112 biv:Ab153” is a bivalent Fc-Fab fusion polypeptide comprising two Fc polypeptides each comprising the sequence of clone 1-112 with LALA and M428L, fused to the high affinity anti-BACE1 Fab domain (Ab153); and “anti-BACE1 control” is a negative control without any TfR-binding site. Chimeric huTfR^{apical} knock-in mice (n=5/group) were intravenously dosed at 50 mg/kg on day 0, day 3, and day 5. All mice were perfused with PBS 24 hours post-dose. Prior to perfusion, blood was collected in EDTA plasma tubes via cardiac puncture and spun at 14,000 rpm for 5 minutes. Plasma was then isolated for subsequent PK and PD analysis. Brains were extracted after perfusion and hemi-brains were isolated for homogenization in 10× by tissue weight of 1% NP-40 in PBS (for PK) or 5 M GuHCl (for PD).

BACE1 inhibition of amyloid precursor protein APP cleavage was used as a pharmacodynamic readout of antibody activity in brain. Brain tissue was homogenized in 10× tissue weight of 5 M guanidine-HCl and then diluted 1:10 in 0.25% casein buffer in PBS. Mouse Aβ40 levels in brain lysate were measured using a sandwich ELISA. A 384-well MaxiSorp plate was coated overnight with a polyclonal capture antibody specific for the C-terminus of the Aβ40 peptide (Millipore #ABN240). Casein-diluted guanidine brain lysates were further diluted 1:2 on the ELISA plate and added concurrently with the detection antibody, biotinylated M3.2. Plasma was analyzed at a 1:5 dilution. Samples were incubated overnight at 4° C. prior to addition of streptavidin-TRP followed by TMB substrate. The standard curve, 0.78-50 μg/mL msAβ40, was fit using a four-parameter logistic regression.

FIG. 46 shows that brain Aβ40 was reduced for mice treated with clone 6.5.11.5.42.2 and clone 1-112 with LALA and M428L.

### Example 22. Methods for Crystal Structure Generation of Clone 1-112 and Circularly Permuted Human TFR Apical Domain Co-Complex

His-tagged circularly permuted human TfR apical domain (huTfR^{apical}) and clone 1-112 Fc with LALA and M428L were expressed in HEK cells at an initial cell density of 2.5×10⁶ cells/mL. Conditioned media was collected 3-4 days post-transfection, and proteins were purified using Ni-NTA (Sigma) or protein A (Genescript) affinity chromatography, as appropriate. Proteins were further purified by size-exclusion chromatography on a Superdex75 16/60 (clone 1-112 with LALA and M428L) and Superdex200 26/60 (huTfR^{apical}) and gel filtration column and eluted in 30 mM Hepes pH 7.4, 150 mM NaCl, 50 mM KCl, 3% glycerol and 1 mM TCEP.

To obtain huTfR^{apical}/clone 1-112 complexes, proteins were mixed with a 1.3 molar excess of huTfR^{apical} and incubated for 1 hour at room temperature. Complexes were purified from excess unbound protein by size-exclusion chromatography on a Superdex200 column and buffer exchanged into 20 mM HEPES pH 7.5, 200 mM NaCl, with 5% glycerol and 1 mM TCEP to a final concentration of 12 mg/mL.

Crystals were grown at 4° C. by the sitting drop vapor diffusion method with a 2:1 mixture of the complex solution and the well solution containing 0.10 M MB2 pH 7.50, 10% amino acid mix (L-Na-Glutamate; Alanine (racemic); Glycine; Lysine HCl (racemic); Serine (racemic)) and 30% w/v PEG550 MME and PEG20k.

Crystals were flash frozen in liquid nitrogen using the crystallization mother liquid supplemented with 25% (v/v) ethylene glycol. Diffraction datasets for the complex were collected at the PX1/XO6SA SWISS LIGHT SOURCE (SLS) using the EIGERX16M detector at 100K. Crystals diffracted to 3.0 Å, and belong to the space group P212121 with two complexes in the same asymmetric unit. Data were indexed, integrated and scaled using the CCP4 suite programs (Xia2-XDS and XSCALE).

### Structure Determination and Refinement

Initial phases of the structure were obtained by molecular replacement using PHASER, and the coordinates of the aglycosylated human Fc fragment (PDB ID: 3S7G) crystal structure were used as the search model. Models were refined by rigid-body refinement followed by restrained refinement using REFMAC. The data collection and refinement statistics are shown in Table 32L. All crystallographic calculations were performed with the CCP4 suite of programs. Model building of the complexes into the electron density was done using the graphics program, COOT.

TABLE 32L

| | Clone 1-112 with LALA and M428L and | |
|---|---|---|
| | Circularly Permuted Human TfR Apical | |
| | Domain Co-complex | |
| Wavelength | 0.999 | |
| Resolution range | 74.86-3.09 (3.34-3.09)* | |
| Space group | P 212121 | |
| Unit cell | 92.78; 116.53; 97.69; | |
| | 90.0; 90.0; 90.0 | |
| Total reflections | 80858 | (16178) |
| Unique reflections | 19639 | (4043) |
| Multiplicity | 4.1 | (4.0) |
| Completeness (%) | 98.2 | (99.2) |
| Mean I/sigma(I) | 11.24 | (1.55) |
| Wilson B-factor | 86.57 | |
| R-merge (%) | 9.6 | (103.8) |
| R-meas (%) | 10.9 | (118.7) |
| R-pim | n.d. | |
| CC½ | 99.9 | (65.7) |

| | |
|---|---|
| CC* | n.d. |
| Reflections used in | 19084 |
| refinement | |
| Reflections used | 555 |
| for R-free | |
| R-work | 21.9 |
| R-free | 27.7 |
| CC(work) | 0.93 (Overall correlation coefficient) |
| CC(free) | 0.91 (Free correlation coefficient |
| Macromolecules | 5403 |
| Protein residues | 703 |
| RMS(bonds) | 0.013 |
| RMS(angles) | 1.739 |
| Ramachandran | 94.1 |
| favored (%) | |
| Ramachandran | 5.18 |
| allowed (%) | |
| Ramachandran | 0.72 |
| outliers (%) | |
| Rotamer outliers (%) | 1.93 |
| Clashscore | 0.57 |
| Macromolecules | 70.58 |
| Average B-factor | 75.9 |

*Statistics for the highest-resolution shell are shown in parentheses.

### Example 23. Crystal Structure of Clone 1-112 Bound to Circularly Permuted Human TFR Apical Domain

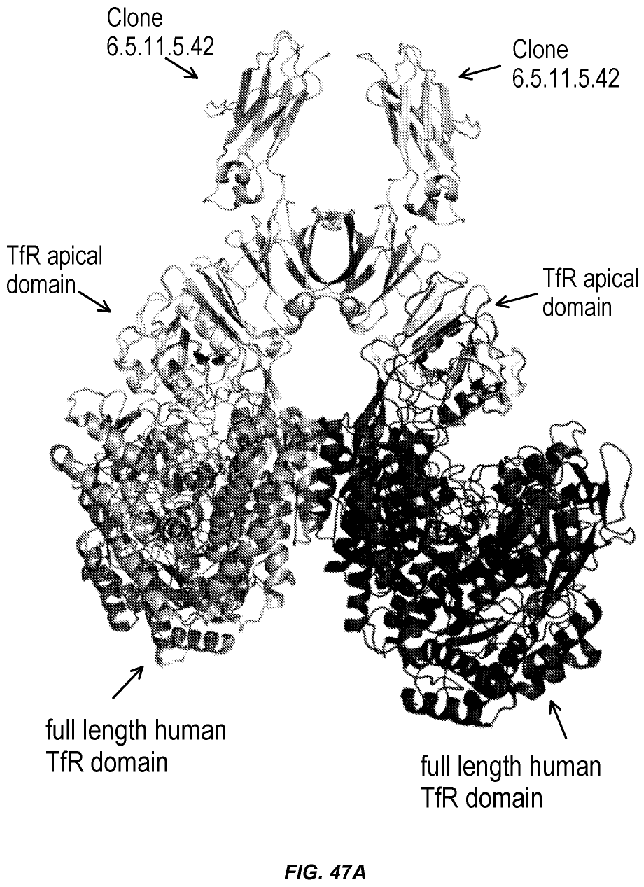

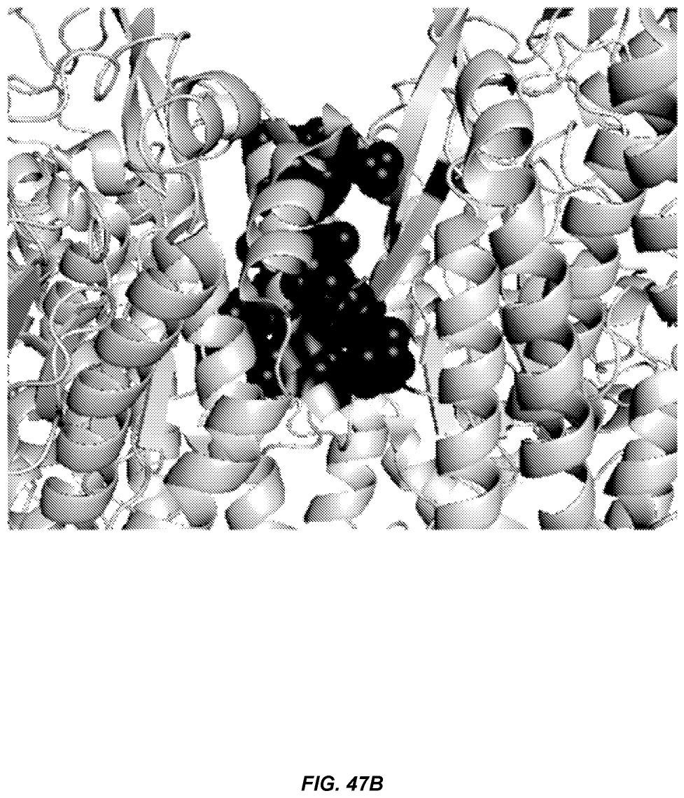

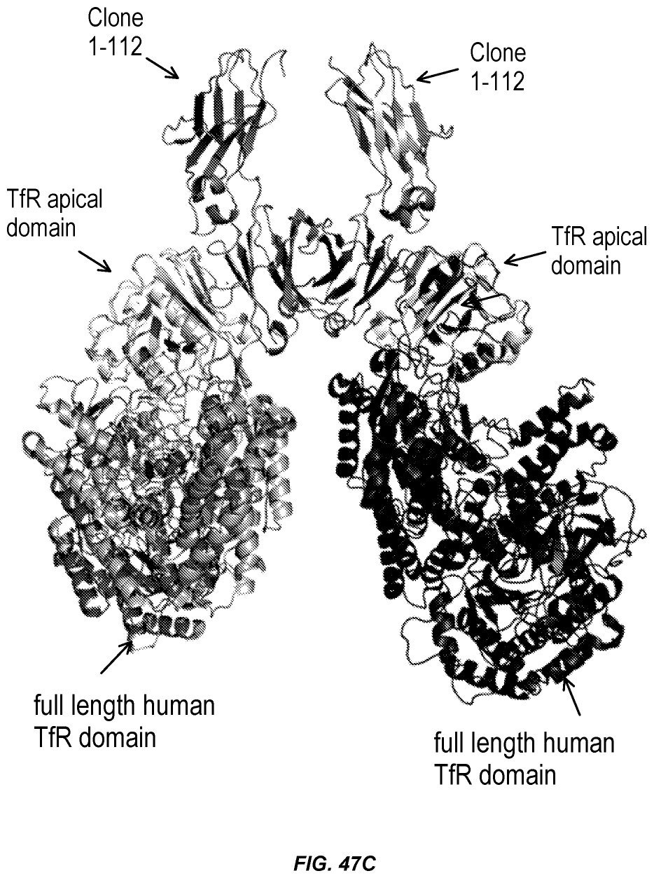

The crystal structure was solved of clone 1-112 with LALA and M428L co-complexed with the human TfR circularly permuted apical domain (FIG. 47C). Clone 1-112 with LALA and M428L binds in a similar manner to the human TfR apical domain as clone 6.5.11.5.42 with the epitope being slightly shifted. Clone 1-112 makes contacts with a beta-sheet surface and surrounding loop regions. When modeling bivalent clone 1-112 or clone 6.5.11.5.42 binding to two full-length TfR ECD, the implications of a slight shift in epitope can be seen as clone 6.5.11.5.42 would cause a steric clash between the two TfR ECD while 1-112 would not (FIGS. 47A-47C). The crystal structures of clone 1-112 and clone 6.5.11.5.42 and TfR ECD modeling suggest that bivalent clone 6.5.11.5.42 would bind full-length TfR in a monovalent fashion while bivalent clone 1-112 would bind full-length TfR in a bivalent fashion under conditions where transferrin is not bound to TfR.

### Example 24. Generation of Tfr Target

### Circularly Permuted Apical Domain

The human and cyno circularly permuted apical domains were cloned into a pET28-Hisio-Smt3-Avi-PreScission-TfR vector, where the TfR residues 326-379 are N-terminal to TfR residues 194-296, to create a circular permutation with new N-terminus and C-teminus and deletion of TfR loop residues 297-325. The sequences for human TfR1 and cyno TfR are shown in SEQ ID NOS:127 and 128, respectively. Apical domain constructs are co-expressed with BirA in BL21(DE3) E. coli cells (Novagen) at 37° C. in LB medium containing antibiotics, until an OD of about 0.7, chilled on ice for 30 minutes, then induced with 1 mM IPTG at 18° C. for 16 hours. Cells are harvested by centrifugation, resuspended into 50 mM Tris-HCl, pH 7.5, 500 mM NaCl, 10% glycerol plus benzonase, incubated for 37° C. for 1 hour, lysed using a microfluidizer, and the insoluble material is removed by centrifugation. The circularly permuted TfR apical domains were purified using a 5 mL His Trap (GeHealthcare), washed with 25 mM and 50 mM imidazole, then eluted with a 100-500 mM imidazole gradient. Fractions were pooled, and cleaved with UlpI at a 100:1 molar ratio, incubated overnight, and dialyzed into 50 mM HEPES, pH 7.5, 150 mM NaCl, 1 mM DTT at 4° C. Cleaved samples were again flowed over an equillibrarted 5 mL HisTrap and the flow through was collected. Samples were further purified by size exclusion chromatography on a Superdex 75 16/60 (GE Healthcare).

### TfR Ectodomain (ECD)

DNA encoding the TfR ectodomain (ECD) (residues 121-760 of the human TfR1 (SEQ ID NO:127) or cyno TfR (SEQ ID NO:128)) was cloned into a mammalian expression vector with C-terminal cleavable His- and Avi-tags. The plasmid was transfected and expressed in HEK293 cells. The ectodomain was purified from the harvested supernatant using Ni-NTA chromatography followed by size-exclusion chromatography to remove any aggregated protein. The yield was about 5 mg per liter of culture. The protein was stored in 10 mM K3PO4 (pH 6.7), 100 mM KCl, 100 mM NaCl, and 20% glycerol and frozen at −20° C.

The purified TfR ECDs were biotinylated using an EZ-link sulfo-NHS-LC-Biotin kit (obtained from Thermo Scientific), using five-fold molar excess of biotin. Excess biotin was removed by extensively dialyzing against PBS.

The Avi-tagged human TfR ECDs and apical domains were biotinylated using BirA-500 (BirA biotin-protein ligase standard reaction kit from Avidity, LLC). After reaction, the labeled proteins were further purified by size-exclusion chromatography to remove excess BirA enzyme. The final material was stored in 10 mM K₃PO₄ (pH 6.7), 100 mM KCl, 100 mM NaCl, and 20% glycerol and frozen at −20° C.

### Full-Length TfR

Full-length human and cyno TfR without Avi-tag was made as previously described in International Patent Publication No. WO 2018/152326.

### Example 25. Methods for Generation and Selection of Yeast Display Libraries

### Library Generation

A DNA template coding for the wild-type human Fc sequence was synthesized and incorporated into a yeast display vector. The Fc polypeptides were displayed on the Aga2p cell wall protein. The vectors contained prepro leader peptides with a Kex2 cleavage sequence, and a c-Myc epitope tag fused to the terminus of the Fc.

Freshly prepared electrocompetent yeast (i.e., strain EBY100) were electroporated with linearized vector and assembled library inserts. After recovery in selective SD-CAA media, the yeast were grown to confluence and split twice, then induced for protein expression by transferring to SG-CAA media. Typical library sizes ranged from about 10⁷ to about 109 transformants. Fc-dimers were formed by pairing of adjacently displayed Fc monomers.

### Library Selection

Magnetic-assisted cell sorting (MACS) and fluorescence-activated cell sorting (FACS) selections were performed similarly to as described in Ackerman, et al. 2009 Biotechnol. Prog. 25(3), 774. Streptavidin magnetic beads (Promega) were labeled with biotinylated target and incubated with yeast (typically 5-10× library diversity). Unbound yeast were removed, the beads were washed, and bound yeast were grown in selective media and induced for subsequent rounds of selection.

For FACS selection, yeast were labeled with anti-c-Myc antibody to monitor expression and biotinylated target (concentration varied depending on the sorting round). In some experiments, the target was pre-mixed with streptavidin-Alexa Fluor® 647 in order to enhance the avidity of the interaction. In other experiments, the biotinylated target was detected after binding and washing with streptavidin-Alexa Fluor® 647. Singlet yeast with binding were sorted using a FACS Aria III cell sorter. The sorted yeast were grown in selective media and then induced for subsequent selection rounds.

After an enriched yeast population was achieved, yeast were plated on SD-CAA agar plates and single colonies were grown and induced for expression, then labeled as described above to determine their propensity to bind to the target. Positive single clones were subsequently sequenced for binding target, after which some clones were expressed either as a soluble Fc fragment or as fused to Fab fragments.

### Example 26. Methods for Cell Uptake

HEK293T, CHO:cyTfR, and CHO cells were plated at 40,000 cells/well of 96-well plates in standard growth media (DMEM (Gibco™ 11995073)+10% FBS (VWR 89510⁻¹⁸⁸)+1×Pen/Strep (Gibco 15140122)). Approximately 24 hours later, molecules were diluted into standard growth media warmed to 37° C. Old media was removed from the cells, and the diluted molecules were added to the cells. Cells were incubated at 37° C. for 45 minutes. Cells were then washed with PBS and then fixed for 10 minutes in 4% PFA (Electron Microscopy Sciences 15714-S). Cells were washed with PBS and then blocked with 5% BSA, 0.3% TritonX100 in PBS for 30 minutes. Cells were stained with anti-human IgG-488 (1:1000; Jackson Immuno Research 109-545-003), cell mask (1:10,000; Thermo H₃₂₇₂₁), and DAPI (1:2000; Thermo D1306) diluted in 1% BSA, 0.3% TritonX100 in PBS for at least 30 minutes. Cells were washed with PBS, imaged on an Opera Phenix, and images were analyzed with Harmony software.

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

Informal Sequence Listing

| SEQ | | |
|---|---|---|
| ID | | |
| NO: | Sequence | Description |
| 1 | APELLGGPSVFLFPPKPKDTLMISRTPEVTCVVVDVSHEDPEVKFNWYVDGVEVHNAKT | Wild-type human Fc |
| | KPREEQYNSTYRVVSVLTVLHQDWLNGKEYKCKVSNKALPAPIEKTISKAKGQPREPQV | sequence |
| | YTLPPSRDELTKNQVSLTCLVKGFYPSDIAVEWESNGQPENNYKTTPPVLDSDGSFFLYSK | |
| | LTVDKSRWQQGNVFSCSVMHEALHNHYTQKSLSLSPGK | |
| 2 | APELLGGPSVFLFPPKPKDTLMISRTPEVTCVVVDVSHEDPEVKFNWYVDGVEVHNAKT | CH2 domain |
| | KPREEQYNSTYRVVSVLTVLHQDWLNGKEYKCKVSNKALPAPIEKTISKAK | sequence |
| 3 | GQPREPQVYTLPPSRDELTKNQVSLTCLVKGFYPSDIAVEWESNGQPENNYKTTPPVLDS | CH3 domain |
| | DGSFFLYSKLTVDKSRWQQGNVFSCSVMHEALHNHYTQKSLSLSPGK | sequence |
| 4 | EPKSCDKTHTCPPCP | human IgG1 hinge |
| 5 | APELLGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | knob mutation |
| | YTLPPSRDELTKNQVSLWCLVKGFYPSDIAVEWESNGQPENNYKTTPPVLDSDGSFFLYS | |
| | KLTVDKSRWQQGNVFSCSVMHEALHNHYTQKSLSLSPGK | |
| 6 | APELLGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | hole mutations |
| | YTLPPSRDELTKNQVSLSCAVKGFYPSDIAVEWESNGQPENNYKTTPPVLDSDGSFFLVS | |
| | KLTVDKSRWQQGNVFSCSVMHEALHNHYTQKSLSLSPGK | |
| 7 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | LALA |
| | YTLPPSRDELTKNQVSLTCLVKGFYPSDIAVEWESNGQPENNYKTTPPVLDSDGSFFLYSK | |
| | LTVDKSRWQQGNVFSCSVMHEALHNHYTQKSLSLSPGK | |
| 8 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | knob and LALA |
| | YTLPPSRDELTKNQVSLWCLVKGFYPSDIAVEWESNGQPENNYKTTPPVLDSDGSFFLYS | |
| | KLTVDKSRWQQGNVFSCSVMHEALHNHYTQKSLSLSPGK | |
| 9 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | hole and LALA |
| | YTLPPSRDELTKNQVSLSCAVKGFYPSDIAVEWESNGQPENNYKTTPPVLDSDGSFFLVS | |
| | KLTVDKSRWQQGNVFSCSVMHEALHNHYTQKSLSLSPGK | |
| 10 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALGAPIEKTISKAKGQPREPQV | LALA and PG |
| | YTLPPSRDELTKNQVSLTCLVKGFYPSDIAVEWESNGQPENNYKTTPPVLDSDGSFFLYSK | |
| | LTVDKSRWQQGNVFSCSVMHEALHNHYTQKSLSLSPGK | |
| 11 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALGAPIEKTISKAKGQPREPQV | knob and LALA and |
| | YTLPPSRDELTKNQVSLWCLVKGFYPSDIAVEWESNGQPENNYKTTPPVLDSDGSFFLYS | PG |
| | KLTVDKSRWQQGNVFSCSVMHEALHNHYTQKSLSLSPGK | |
| 12 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALGAPIEKTISKAKGQPREPQV | hole and LALA and |
| | YTLPPSRDELTKNQVSLSCAVKGFYPSDIAVEWESNGQPENNYKTTPPVLDSDGSFFLVS | PG |
| | KLTVDKSRWQQGNVFSCSVMHEALHNHYTQKSLSLSPGK | |
| 13 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALSAPIEKTISKAKGQPREPQV | LALA and PS |
| | YTLPPSRDELTKNQVSLTCLVKGFYPSDIAVEWESNGQPENNYKTTPPVLDSDGSFFLYSK | |
| | LTVDKSRWQQGNVFSCSVMHEALHNHYTQKSLSLSPGK | |
| 14 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALSAPIEKTISKAKGQPREPQV | knob and LALA and |
| | YTLPPSRDELTKNQVSLWCLVKGFYPSDIAVEWESNGQPENNYKTTPPVLDSDGSFFLYS | PS |
| | KLTVDKSRWQQGNVFSCSVMHEALHNHYTQKSLSLSPGK | |
| 15 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALSAPIEKTISKAKGQPREPQV | hole and LALA and |
| | YTLPPSRDELTKNQVSLSCAVKGFYPSDIAVEWESNGQPENNYKTTPPVLDSDGSFFLVS | PS |
| | KLTVDKSRWQQGNVFSCSVMHEALHNHYTQKSLSLSPGK | |
| 16 | APELLGGPSVFLFPPKPKDTLMISRTPEVTCVVVDVSHEDPEVKFNWYVDGVEVHNAKT | Fc sequence with LS |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWESNGQPENNYKTTPPVLDSDGSFFLYSK | |
| | LTVDKSRWQQGNVFSCSVLHEALHSHYTQKSLSLSPGK | |
| 17 | APELLGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | knob and LS |
| | YTLPPSRDELTKNQVSLWCLVKGFYPSDIAVEWESNGQPENNYKTTPPVLDSDGSFFLYS | |
| | KLTVDKSRWQQGNVFSCSVLHEALHSHYTQKSLSLSPGK | |
| 18 | APELLGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | hole and LS |
| | YTLPPSRDELTKNQVSLSCAVKGFYPSDIAVEWESNGQPENNYKTTPPVLDSDGSFFLVS | |
| | KLTVDKSRWQQGNVFSCSVLHEALHSHYTQKSLSLSPGK | |
| 19 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | LALA and LS |
| | YTLPPSRDELTKNQVSLTCLVKGFYPSDIAVEWESNGQPENNYKTTPPVLDSDGSFFLYSK | |
| | LTVDKSRWQQGNVFSCSVLHEALHSHYTQKSLSLSPGK | |
| 20 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | knob and LALA and |
| | YTLPPSRDELTKNQVSLWCLVKGFYPSDIAVEWESNGQPENNYKTTPPVLDSDGSFFLYS | LS |
| | KLTVDKSRWQQGNVFSCSVLHEALHSHYTQKSLSLSPGK | |
| 21 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALPAPIEKTISKAKGQPREPQV | hole and LALA and |
| | YTLPPSRDELTKNQVSLSCAVKGFYPSDIAVEWESNGQPENNYKTTPPVLDSDGSFFLVS | LS |
| | KLTVDKSRWQQGNVFSCSVLHEALHSHYTQKSLSLSPGK | |
| 22 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALGAPIEKTISKAKGQPREPQV | LALA and PG and |
| | YTLPPSRDELTKNQVSLTCLVKGFYPSDIAVEWESNGQPENNYKTTPPVLDSDGSFFLYSK | LS |
| | LTVDKSRWQQGNVFSCSVLHEALHSHYTQKSLSLSPGK | |
| 23 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALGAPIEKTISKAKGQPREPQV | knob and LALA and |
| | YTLPPSRDELTKNQVSLWCLVKGFYPSDIAVEWESNGQPENNYKTTPPVLDSDGSFFLYS | PG and LS |
| | KLTVDKSRWQQGNVFSCSVLHEALHSHYTQKSLSLSPGK | |
| 24 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALGAPIEKTISKAKGQPREPQV | hole and LALA and |
| | YTLPPSRDELTKNQVSLSCAVKGFYPSDIAVEWESNGQPENNYKTTPPVLDSDGSFFLVS | PG and LS |
| | KLTVDKSRWQQGNVFSCSVLHEALHSHYTQKSLSLSPGK | |
| 25 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALSAPIEKTISKAKGQPREPQV | LALA and PS and LS |
| | YTLPPSRDELTKNQVSLTCLVKGFYPSDIAVEWESNGQPENNYKTTPPVLDSDGSFFLYSK | |
| | LTVDKSRWQQGNVFSCSVLHEALHSHYTQKSLSLSPGK | |
| 26 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALSAPIEKTISKAKGQPREPQV | knob and LALA and |
| | YTLPPSRDELTKNQVSLWCLVKGFYPSDIAVEWESNGQPENNYKTTPPVLDSDGSFFLYS | PS and LS |
| | KLTVDKSRWQQGNVFSCSVLHEALHSHYTQKSLSLSPGK | |
| 27 | APEAAGGPSVFLFPPKPKDTLMISRTPEVTCVVVDVSHEDPEVKFNWYVDGVEVHNAKT | Fc sequence with |
| | KPREEQYNSTYRVVSVLTVLHQDWLNGKEYKCKVSNKALSAPIEKTISKAKGQPREPQV | hole and LALA and |
| | YTLPPSRDELTKNQVSLSCAVKGFYPSDIAVEWESNGQPENNYKTTPPVLDSDGSFFLVS | PS and LS |
| | KLTVDKSRWQQGNVFSCSVLHEALHSHYTQKSLSLSPGK | |
| 28 | APELLGGPSVFLFPPKPKDTLMISRTPEVTCVVVDVSHEDPEVKFNWYVDGVEVHNAKT | Clone LLB2-10-6 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVLWNSRFVLENNYKTTPPVLDSDGSFFLYSK | |
| | LTVDKSRWQQGNIFACNVYHEALHNHYTFKNLALSPGK | |
| 29 | APELLGGPSVFLFPPKPKDTLMISRTPEVTCVVVDVSHEDPEVKFNWYVDGVEVHNAKT | Clone LLB2-10-8 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVLWNSRFVLENNYKTTPPVLDSDGSFFLYSK | |
| | LTVDKSRWQQGEIFACNVYHEALHNHYTFKNLALSPGK | |
| 30 | APELLGGPSVFLFPPKPKDTLMISRTPEVTCVVVDVSHEDPEVKFNWYVDGVEVHNAKT | Clone LLB2-10-8- |
| | KPREEQYNSTYRVVSVLTVLHQDWLNGKEYKCKVSNKALPAPIEKTISKAKGQPREPQV | d18 |
| | YTLPPSRDELTKNQVSLTCLVKGFYPSDIAVLWNSQYELENNYKTTPPVLDSDGSFFLYS | |
| | KLTVDKSRWQQGNVFACNVYHEALHNHYTFKNLALSPGK | |
| 31 | APELLGGPSVFLFPPKPKDTLMISRTPEVTCVVVDVSHEDPEVKFNWYVDGVEVHNAKT | Clone LLB2-10-8- |
| | KPREEQYNSTYRVVSVLTVLHQDWLNGKEYKCKVSNKALPAPIEKTISKAKGQPREPQV | d12 |
| | YTLPPSRDELTKNQVSLTCLVKGFYPSDIAVLWNSHYELENNYKTTPPVLDSDGSFFLYS | |
| | KLTVDKSRWQQGNVFACNVYHEALHNHYTFKNLALSPGK | |
| 32 | APELLGGPSVFLFPPKPKDTLMISRTPEVTCVVVDVSHEDPEVKFNWYVDGVEVHNAKT | Clone LLB2-10-8-d6 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVLWNSRFVLENNYKTTPPVLDSDGSFFLYSK | |
| | LTVDKSRWQQGNVFACNVYHEALHNHYTFKNLALSPGK | |
| 33 | APELLGGPSVFLFPPKPKDTLMISRTPEVTCVVVDVSHEDPEVKFNWYVDGVEVHNAKT | Clone LLB2-10-8-d3 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVLWNSRFVLENNYKTTPPVLDSDGSFFLYSK | |
| | LTVDKSRWQQGEVFACNVYHEALHNHYTFKNLALSPGK | |
| 34 | APELLGGPSVFLFPPKPKDTLMISRTPEVTCVVVDVSHEDPEVKFNWYVDGVEVHNAKT | Clone LLB2-10-8-d1 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVLWNSRFVLENNYKTTPPVLDSDGSFFLYSK | |
| | LTVDKSRWQQGEIFACNVYHEALHNHYTFKNLSLSPGK | |
| 35 | APELLGGPSVFLFPPKPKDTLMISRTPEVTCVVVDVSHEDPEVKFNWYVDGVEVHNAKT | Clone |
| | KPREEQYNSTYRVVSVLTVLHQDWLNGKEYKCKVSNKALPAPIEKTISKAKGQPREPQV | LLB2.10.8.10.3 |
| | YTLPPSRDELTKNQVSLTCLVKGFYPSDIAVLWNSRFVLENNYKTTPPVLDSDGSFFLYSK | |
| | LTVDKSRWQQGEIFACNVYHEALHNHYTFKNLRLSPGK | |
| 36 | APELLGGPSVFLFPPKPKDTLMISRTPEVTCVVVDVSHEDPEVKFNWYVDGVEVHNAKT | Clone |
| | KPREEQYNSTYRVVSVLTVLHQDWLNGKEYKCKVSNKALPAPIEKTISKAKGQPREPQV | LLB2.10.8.10.8 |
| | YTLPPSRDELTKNQVSLTCLVKGFYPSDIAVLWNSRFVLENNYKTTPPVLDSDGSFFLYSK | |
| | LTVDKSRWQQGEIFACNVYHEALHNHYTFKNLHLSPGK | |
| 37 | APELLGGPSVFLFPPKPKDTLMISRTPEVTCVVVDVSHEDPEVKFNWYVDGVEVHNAKT | Clone |
| | KPREEQYNSTYRVVSVLTVLHQDWLNGKEYKCKVSNKALPAPIEKTISKAKGQPREPQV | LLB2.10.8.14.3 |
| | YTLPPSRDELTKNQVSLTCLVKGFYPSDIAVLWNSRFVLENNYKTTPPVLDSDGSFFLYSK | |
| | LTVDKSRWQQGEIFACNVYHEALHNHRTFKNLRLSPGK | |
| 38 | APELLGGPSVFLFPPKPKDTLMISRTPEVTCVVVDVSHEDPEVKFNWYVDGVEVHNAKT | Clone |
| | KPREEQYNSTYRVVSVLTVLHQDWLNGKEYKCKVSNKALPAPIEKTISKAKGQPREPQV | LLB2.10.8.12.5 |
| | YTLPPSRDELTKNQVSLTCLVKGFYPSDIAVLWNSHYELENNYKTTPPVLDSDGSFFLYS | |
| | KLTVDKSRWQQGEIFACNVYHEALHNHYTFKNLALSPGK | |
| 39 | APELLGGPSVFLFPPKPKDTLMISRTPEVTCVVVDVSHEDPEVKFNWYVDGVEVHNAKT | Clone LLB2-37 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVLWNSQFHLENNYKTTPPVLDSDGSFFLYSK | |
| | LTVDKSRWQQGNIFACNVYHEALHNHYTFKNLLLSPGK | |
| 40 | APELLGGPSVFLFPPKPKDTLMISRTPEVTCVVVDVSHEDPEVKFNWYVDGVEVHNAKT | Clone |
| | KPREEQYNSTYRVVSVLTVLHQDWLNGKEYKCKVSNKALPAPIEKTISKAKGQPREPQV | LLB2.10.8.9.11.N |
| | YTLPPSRDELTKNQVSLTCLVKGFYPSDIAVLWNSRFVLENNYKTTPPVLDSDGSFFLYSK | |
| | LTVDKSRWQQGNTFACNVYHEALHNHYTFKNLALSPGK | |
| 41 | APELLGGPSVFLFPPKPKDTLMISRTPEVTCVVVDVSHEDPEVKFNWYVDGVEVHNAKT | Clone |
| | KPREEQYNSTYRVVSVLTVLHQDWLNGKEYKCKVSNKALPAPIEKTISKAKGQPREPQV | LLB2.10.8.10.1 |
| | YTLPPSRDELTKNQVSLTCLVKGFYPSDIAVLWNSRFVLENNYKTTPPVLDSDGSFFLYSK | |
| | LTVDKSRWQQGEIFACNVYHEALHNHYTFKNLKLSPGK | |
| 42 | APELLGGPSVFLFPPKPKDTLMISRTPEVTCVVVDVSHEDPEVKFNWYVDGVEVHNAKT | Clone |
| | KPREEQYNSTYRVVSVLTVLHQDWLNGKEYKCKVSNKALPAPIEKTISKAKGQPREPQV | LLB2.10.8.4.12 |
| | YTLPPSRDELTKNQVSLTCLVKGFYPSDIAVLWNSRFVLENNYKTTPPVLDSDGSFFLYSK | |
| | LTVDKSRWQQGEIFACNVYHEALHNHWTFKNLRLSPGK | |
| 43 | APELLGGPSVFLFPPKPKDTLMISRTPEVTCVVVDVSHEDPEVKFNWYVDGVEVHNAKT | Clone LLB2.10.8.2.1 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVLWNSQYLLENNYKTTPPVLDSDGSFFLYS | |
| | KLTVDKSRWQQGEIFACNVYHEALHNHYTFKNLALSPGK | |
| 44 | APELLGGPSVFLFPPKPKDTLMISRTPEVTCVVVDVSHEDPEVKFNWYVDGVEVHNAKT | Clone LLB1-3-16-2 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWRTYKPYETNYKTTPPVLDSDGSFFLYSK | |
| | LTVDKSRWQQGDIFVCDVMHEALHNHFTIKKLSLSPGK | |
| 45 | APELLGGPSVFLFPPKPKDTLMISRTPEVTCVVVDVSHEDPEVKFNWYVDGVEVHNAKT | Clone LLB1-3-16 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWRTYKPYETNYKTTPPVLDSDGSFFLYSK | |
| | LTVDKSRWQQGDIFVCDVLHEALHNHFTIKKLSLSPGK | |
| 46 | LX₁NX₂X₃X₄X₅L, in which X₁ is any amino acid; X₂ is R, H, or Q; X₃ | LLB2 Consensus |
| | is F or Y; X₄ is V, L, I, F, Y, or E; and Xsis any amino acid | 380-387 |
| 47 | X₁X₂X₃AX₄X₅X₆X₇, in which X₁ is E, N, Q, or A; X₂ is I, V, T, or P; | LLB2 Consensus |
| | X₃ and X₄ are any amino acid; X₅ is N or S; X₆ is any amino acid, | 421-428 |
| | and X₇ is Y or W | |
| 48 | X₁X₂X₃X₄NX₅X₆, in which X₁ is Y, R, or W; X₂ is any amino acid; X₃ | LLB2 Consensus |
| | is F or W; X₄ and X₅ are any amino acid; and X₆ is A, Q, K, R, | 436-442 |
| | H, M, or S | |
| 49 | X₁X₂YKPYX₃T, in which X₁ is E or R; X₂ is S or T; and X₃ is any | LLB1 Consensus |
| | amino acid | 382-389 |
| 50 | X₁X₂X₃VX₄DX₅X₆, in which X₁ is N or D; X₂ is V or I; X₃, X₄, and X₅ | LLB1 Consensus |
| | and are any amino acid; X₆ is M or L | 421-428 |
| 51 | X₁X₂IX₃X₄, in which X₁ is Y or F; X₂ and X₃ are any amino acid; | LLB1 Consensus |
| | and X₄ is S or K. | 436-440 |
| 52 | EWESNGQP | 380 to position 387 |
| | | of a wild-type Fc |
| | | polypeptide |
| 53 | NVFSCSVM | 421 to position 428 |
| | | of the wild-type Fc |
| | | polypeptide |
| 54 | YTQKSLS | 436 to position 442 |
| | | of the wild-type Fc |
| | | polypeptide |
| 55 | MELQPPEASIAVVSIPRQLPGSHSEAGVQGLSAGDDSELGSHCVAQTGLELLASGDPLPSA | Human CD98hc |
| | SQNAEMIETGSDCVTQAGLQLLASSDPPALASKNAEVTGTMSQDTEVDMKEVELNELEP | |
| | EKQPMNAASGAAMSLAGAEKNGLVKIKVAEDEAEAAAAAKFTGLSKEELLKVAGSPGW | |
| | VRTRWALLLLFWLGWLGMLAGAVVIIVRAPRCRELPAQKWWHTGALYRIGDLQAFQGH | |
| | GAGNLAGLKGRLDYLSSLKVKGLVLGPIHKNQKDDVAQTDLLQIDPNFGSKEDFDSLLQ | |
| | SAKKKSIRVILDLTPNYRGENSWFSTQVDTVATKVKDALEFWLQAGVDGFQVRDIENLK | |
| | DASSFLAEWQNITKGFSEDRLLIAGTNSSDLQQILSLLESNKDLLLTSSYLSDSGSTGEHTK | |
| | SLVTQYLNATGNRWCSWSLSQARLLTSFLPAQLLRLYQLMLFTLPGTPVFSYGDEIGLDA | |
| | AALPGQPMEAPVMLWDESSFPDIPGAVSANMTVKGQSEDPGSLLSLFRRLSDQRSKERSL | |
| | LHGDFHAFSAGPGLFSYIRHWDQNERFLVVLNFGDVGLSAGLQASDLPASASLPAKADL | |
| | LLSTQPGREEGSPLELERLKLEPHEGLLLRFPYAA | |
| 56 | AVEWESNGQPENN | 378-390 of wild-type |
| | | Fc |
| 57 | VFSCSVMHEALHNHYTQKS | 422-440 of wild-type |
| | | Fc |
| 58 | AVEWFYDDSKLTN | 42.2.19 aa 378-390 |
| 59 | AVEWFYGNAKETN | 42.2.3-1H aa 378-390 |
| 60 | AVEWFYEAQKLNN | 42.8.196 aa 378-390 |
| 61 | AVEWFSEGSKETN | 42.8.80 aa 378-390 |
| 62 | AVEWFSGAQKESN | 42.8.15 aa 378-390 |
| 63 | AVEWFSGAQKLTN | 42.8.17 aa 378-390 |
| 64 | LFACEVMHEALHNHYTYKL | 42.2.3-1H aa 422-440 |
| | | 42.8.196 aa 422-440 |
| | | 42.8.80 aa 422-440 |
| | | 42.8.15 aa 422-440 |
| | | 42.8.17 aa 422-440 |
| 65 | AVX₁WFX₂X₃X₄X₅X₆X₇X&N, wherein X₁ is E, N, F, or Y; X₂ is Y, S, A, | aa 378-390 consensus |
| | or absent; X₃ is G, D, E, or N; X₄ is D, G, N, or A; X₅ is Q, S, | sequence |
| | G, A, or N; X₆ is K, I, R, or G; X₇ is E, L, D, or Q; and X₈ is | |
| | N, T, S, or R | |
| 66 | AVEWFX₁X₂X₃X₄KX₅X₆N, wherein X₁ is Y or S; X₂ is G, D, or E; X₃ is | aa 378-390 consensus |
| | D, G, N, or A; X₄ is Q, S, or A; X₅ is E or L; and X₆ is N, T, | sequence |
| | or S | |
| 67 | LFACEVMHEALX₁X₂HYTYKL, wherein X₁ is H or E; and X₂ is N or G | aa 422-440 consensus |
| | | sequence |
| 68 | APELLGGPSVFLFPPKPKDTLMISRTPEVTCVVVDVSHEDPEVKFNWYVDGVEVHNAKT | Fc consensus |
| | KPREEQYNSTYRVVSVLTVLHQDWLNGKEYKCKVSNKALPAPIEKTISKAKGQPREPQV | sequence |
| | YTLPPSRDELTKNQVSLTCLVKGFYPSDIAVX₁WFX₂X₃X₄X₅X₆X₇X₈NYKTTPPVLDSDGSF | |
| | FLYSKLTVDKSRWQX₉X₁₀X₁₁LFACEVMHEALX₁₂X₁₃HYTYKLLX₁₄X₁₅SPGK, wherein X₁ | |
| | is E, N, F, or Y; X₂ is Y, S, A, or absent; X₃ is G, D, E, or N; | |
| | X₄ is D, G, N, or A; X₅ is Q, S, G, A, or N; X₆ is K, I, R, or G; | |
| | X₇ is E, L, D, or Q; X₈ is N, T, S, or R; X₉ is Q or P; X₁₀ is G | |
| | or R; X₁₁ is N or G; X₁₂ is H or E; X₁₃ is N or G; X₁₄ is S or | |
| | G; and X₁₅ is L or E | |
| 69 | APELLGGPSVFLFPPKPKDTLMISRTPEVTCVVVDVSHEDPEVKFNWYVDGVEVHNAKT | Fc consensus |
| | KPREEQYNSTYRVVSVLTVLHQDWLNGKEYKCKVSNKALPAPIEKTISKAKGQPREPQV | sequence |
| | YTLPPSRDELTKNQVSLTCLVKGFYPSDIAVX₁WFX₂X₃X₄X₅X₆X₇X₈NYKTTPPVLDSDGSF | |
| | FLYSKLTVDKSRWQX₉X₁₀X₁₁LFACEVMHEALHNHYTYKLLX₁₂X₁₃SPGK, wherein X₁ is | |
| | E, N, F, or Y; X₂ is Y, S, A, or absent; X₃ is G, D, E, or N; X₄ | |
| | is D, G, N, or A; X₅ is Q, S, G, A, or N; X₆ is K, I, R, or G; | |
| | X₇ is E, L, D, or Q; X₈ is N, T, S, or R; X₉ is Q or P; X₁₀ is G | |
| | or R; X₁₁ is N or G; X₁₂ is S or G; and X₁₃ is L or E | |
| 70 | APELLGGPSVFLFPPKPKDTLMISRTPEVTCVVVDVSHEDPEVKFNWYVDGVEVHNAKT | Fc consensus |
| | KPREEQYNSTYRVVSVLTVLHQDWLNGKEYKCKVSNKALPAPIEKTISKAKGQPREPQV | sequence |
| | YTLPPSRDELTKNQVSLTCLVKGFYPSDIAVX₁WFX₂X₃X₄X₅X₆X₇X₈NYKTTPPVLDSDGSF | |
| | FLYSKLTVDKSRWQQGNLFACEVMHEALHNHYTYKLLSLSPGK, wherein X₁ is E, N, | |
| | F, or Y; X₂ is Y, S, A, or absent; X₃ is G, D, E, or N; X₄ is D, | |
| | G, N, or A; X₅ is Q, S, G, A, or N; X₆ is K, I, R, or G; X₇ | |
| | is E, L, D, or Q; and X₈ is N, T, S, or R. | |
| 71 | APELLGGPSVFLFPPKPKDTLMISRTPEVTCVVVDVSHEDPEVKFNWYVDGVEVHNAKT | Fc consensus |
| | KPREEQYNSTYRVVSVLTVLHQDWLNGKEYKCKVSNKALPAPIEKTISKAKGQPREPQV | sequence |
| | YTLPPSRDELTKNQVSLTCLVKGFYPSDIAVEWFX₁X₂X₃X₄KX₅X₆NYKTTPPVLDSDGSFF | |
| | LYSKLTVDKSRWQQGNLFACEVMHEALHNHYTYKLLSLSPGK, wherein X₁ is Y or | |
| | S; X₂ is G, D, or E; X₃ is D, G, N, or A; X₄ is Q, S, or A; | |
| | X₅ is E or L; and X₆ is N, T, or S | |
| 72 | APELLGGPSVFLFPPKPKDTLMISRTPEVTCVVVDVSHEDPEVKFNWYVDGVEVHNAKT | 42.2.19 Fc |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFYDDSKLTNYKTTPPVLDSDGSFFLYSK | |
| | LTVDKSRWQPRGLFACEVMHEALHNHYTYKLLGESPGK | |
| 73 | APELLGGPSVFLFPPKPKDTLMISRTPEVTCVVVDVSHEDPEVKFNWYVDGVEVHNAKT | 42.2.3-1H Fc |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFYGNAKETNYKTTPPVLDSDGSFFLYS | |
| | KLTVDKSRWQQGNLFACEVMHEALHNHYTYKLLSLSPGK | |
| 74 | APELLGGPSVFLFPPKPKDTLMISRTPEVTCVVVDVSHEDPEVKFNWYVDGVEVHNAKT | 42.8.196 Fc |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFYEAQKLNNYKTTPPVLDSDGSFFLYS | |
| | KLTVDKSRWQQGNLFACEVMHEALHNHYTYKLLSLSPGK | |
| 75 | APELLGGPSVFLFPPKPKDTLMISRTPEVTCVVVDVSHEDPEVKFNWYVDGVEVHNAKT | 42.8.80 Fc |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFSEGSKETNYKTTPPVLDSDGSFFLYSK | |
| | LTVDKSRWQQGNLFACEVMHEALHNHYTYKLLSLSPGK | |
| 76 | APELLGGPSVFLFPPKPKDTLMISRTPEVTCVVVDVSHEDPEVKFNWYVDGVEVHNAKT | 42.8.15 Fc |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFSGAQKESNYKTTPPVLDSDGSFFLYSK | |
| | LTVDKSRWQQGNLFACEVMHEALHNHYTYKLLSLSPGK | |
| 77 | APELLGGPSVFLFPPKPKDTLMISRTPEVTCVVVDVSHEDPEVKFNWYVDGVEVHNAKT | 42.8.17 Fc |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFSGAQKLTNYKTTPPVLDSDGSFFLYSK | |
| | LTVDKSRWQQGNLFACEVMHEALHNHYTYKLLSLSPGK | |
| 78 | APELLGGPSVFLFPPKPKDTLMISRTPEVTCVVVDVSHEDPEVKFNWYVDGVEVHNAKT | 42.2.19 Fc knob |
| | KPREEQYNSTYRVVSVLTVLHQDWLNGKEYKCKVSNKALPAPIEKTISKAKGQPREPQV | |
| | YTLPPSRDELTKNQVSLWCLVKGFYPSDIAVEWFYDDSKLTNYKTTPPVLDSDGSFFLYS | |
| | KLTVDKSRWQPRGLFACEVMHEALHNHYTYKLLGESPGK | |
| 79 | APELLGGPSVFLFPPKPKDTLMISRTPEVTCVVVDVSHEDPEVKFNWYVDGVEVHNAKT | 42.2.3-1H Fc knob |
| | KPREEQYNSTYRVVSVLTVLHQDWLNGKEYKCKVSNKALPAPIEKTISKAKGQPREPQV | |
| | YTLPPSRDELTKNQVSLWCLVKGFYPSDIAVEWFYGNAKETNYKTTPPVLDSDGSFFLYS | |
| | KLTVDKSRWQQGNLFACEVMHEALHNHYTYKLLSLSPGK | |
| 80 | APELLGGPSVFLFPPKPKDTLMISRTPEVTCVVVDVSHEDPEVKFNWYVDGVEVHNAKT | 42.8.196 Fc knob |
| | KPREEQYNSTYRVVSVLTVLHQDWLNGKEYKCKVSNKALPAPIEKTISKAKGQPREPQV | |
| | YTLPPSRDELTKNQVSLWCLVKGFYPSDIAVEWFYEAQKLNNYKTTPPVLDSDGSFFLYS | |
| | KLTVDKSRWQQGNLFACEVMHEALHNHYTYKLLSLSPGK | |
| 81 | APELLGGPSVFLFPPKPKDTLMISRTPEVTCVVVDVSHEDPEVKFNWYVDGVEVHNAKT | 42.8.80 Fc knob |
| | KPREEQYNSTYRVVSVLTVLHQDWLNGKEYKCKVSNKALPAPIEKTISKAKGQPREPQV | |
| | YTLPPSRDELTKNQVSLWCLVKGFYPSDIAVEWFSEGSKETNYKTTPPVLDSDGSFFLYS | |
| | KLTVDKSRWQQGNLFACEVMHEALHNHYTYKLLSLSPGK | |
| 82 | APELLGGPSVFLFPPKPKDTLMISRTPEVTCVVVDVSHEDPEVKFNWYVDGVEVHNAKT | 42.8.15 Fc knob |
| | KPREEQYNSTYRVVSVLTVLHQDWLNGKEYKCKVSNKALPAPIEKTISKAKGQPREPQV | |
| | YTLPPSRDELTKNQVSLWCLVKGFYPSDIAVEWFSGAQKESNYKTTPPVLDSDGSFFLYS | |
| | KLTVDKSRWQQGNLFACEVMHEALHNHYTYKLLSLSPGK | |
| 83 | APELLGGPSVFLFPPKPKDTLMISRTPEVTCVVVDVSHEDPEVKFNWYVDGVEVHNAKT | 42.8.17 Fc knob |
| | KPREEQYNSTYRVVSVLTVLHQDWLNGKEYKCKVSNKALPAPIEKTISKAKGQPREPQV | |
| | YTLPPSRDELTKNQVSLWCLVKGFYPSDIAVEWFSGAQKLTNYKTTPPVLDSDGSFFLYS | |
| | KLTVDKSRWQQGNLFACEVMHEALHNHYTYKLLSLSPGK | |
| 84 | APELLGGPSVFLFPPKPKDTLMISRTPEVTCVVVDVSHEDPEVKFNWYVDGVEVHNAKT | 42.2.19 Fc hole |
| | KPREEQYNSTYRVVSVLTVLHQDWLNGKEYKCKVSNKALPAPIEKTISKAKGQPREPQV | |
| | YTLPPSRDELTKNQVSLSCAVKGFYPSDIAVEWFYDDSKLTNYKTTPPVLDSDGSFFLVS | |
| | KLTVDKSRWQPRGLFACEVMHEALHNHYTYKLLGESPGK | |
| 85 | APELLGGPSVFLFPPKPKDTLMISRTPEVTCVVVDVSHEDPEVKFNWYVDGVEVHNAKT | 42.2.3-1H Fc hole |
| | KPREEQYNSTYRVVSVLTVLHQDWLNGKEYKCKVSNKALPAPIEKTISKAKGQPREPQV | |
| | YTLPPSRDELTKNQVSLSCAVKGFYPSDIAVEWFYGNAKETNYKTTPPVLDSDGSFFLVS | |
| | KLTVDKSRWQQGNLFACEVMHEALHNHYTYKLLSLSPGK | |
| 86 | APELLGGPSVFLFPPKPKDTLMISRTPEVTCVVVDVSHEDPEVKFNWYVDGVEVHNAKT | 42.8.196 Fc hole |
| | KPREEQYNSTYRVVSVLTVLHQDWLNGKEYKCKVSNKALPAPIEKTISKAKGQPREPQV | |
| | YTLPPSRDELTKNQVSLSCAVKGFYPSDIAVEWFYEAQKLNNYKTTPPVLDSDGSFFLVS | |
| | KLTVDKSRWQQGNLFACEVMHEALHNHYTYKLLSLSPGK | |
| 87 | APELLGGPSVFLFPPKPKDTLMISRTPEVTCVVVDVSHEDPEVKFNWYVDGVEVHNAKT | 42.8.80 Fc hole |
| | KPREEQYNSTYRVVSVLTVLHQDWLNGKEYKCKVSNKALPAPIEKTISKAKGQPREPQV | |
| | YTLPPSRDELTKNQVSLSCAVKGFYPSDIAVEWFSEGSKETNYKTTPPVLDSDGSFFLVSK | |
| | LTVDKSRWQQGNLFACEVMHEALHNHYTYKLLSLSPGK | |
| 88 | APELLGGPSVFLFPPKPKDTLMISRTPEVTCVVVDVSHEDPEVKFNWYVDGVEVHNAKT | 42.8.15 Fc hole |
| | KPREEQYNSTYRVVSVLTVLHQDWLNGKEYKCKVSNKALPAPIEKTISKAKGQPREPQV | |
| | YTLPPSRDELTKNQVSLSCAVKGFYPSDIAVEWFSGAQKESNYKTTPPVLDSDGSFFLVSK | |
| | LTVDKSRWQQGNLFACEVMHEALHNHYTYKLLSLSPGK | |
| 89 | APELLGGPSVFLFPPKPKDTLMISRTPEVTCVVVDVSHEDPEVKFNWYVDGVEVHNAKT | 42.8.17 Fc hole |
| | KPREEQYNSTYRVVSVLTVLHQDWLNGKEYKCKVSNKALPAPIEKTISKAKGQPREPQV | |
| | YTLPPSRDELTKNQVSLSCAVKGFYPSDIAVEWFSGAQKLTNYKTTPPVLDSDGSFFLVS | |
| | KLTVDKSRWQQGNLFACEVMHEALHNHYTYKLLSLSPGK | |
| 90 | APEAAGGPSVFLFPPKPKDTLMISRTPEVTCVVVDVSHEDPEVKFNWYVDGVEVHNAKT | 42.2.19 Fc LALA |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFYDDSKLTNYKTTPPVLDSDGSFFLYSK | |
| | LTVDKSRWQPRGLFACEVMHEALHNHYTYKLLGESPGK | |
| 91 | APEAAGGPSVFLFPPKPKDTLMISRTPEVTCVVVDVSHEDPEVKFNWYVDGVEVHNAKT | 42.2.3-1H Fc LALA |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFYGNAKETNYKTTPPVLDSDGSFFLYS | |
| | KLTVDKSRWQQGNLFACEVMHEALHNHYTYKLLSLSPGK | |
| 92 | APEAAGGPSVFLFPPKPKDTLMISRTPEVTCVVVDVSHEDPEVKFNWYVDGVEVHNAKT | 42.8.196 Fc LALA |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFYEAQKLNNYKTTPPVLDSDGSFFLYS | |
| | KLTVDKSRWQQGNLFACEVMHEALHNHYTYKLLSLSPGK | |
| 93 | APEAAGGPSVFLFPPKPKDTLMISRTPEVTCVVVDVSHEDPEVKFNWYVDGVEVHNAKT | 42.8.80 Fc LALA |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFSEGSKETNYKTTPPVLDSDGSFFLYSK | |
| | LTVDKSRWQQGNLFACEVMHEALHNHYTYKLLSLSPGK | |
| 94 | APEAAGGPSVFLFPPKPKDTLMISRTPEVTCVVVDVSHEDPEVKFNWYVDGVEVHNAKT | 42.8.15 Fc LALA |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFSGAQKESNYKTTPPVLDSDGSFFLYSK | |
| | LTVDKSRWQQGNLFACEVMHEALHNHYTYKLLSLSPGK | |
| 95 | APEAAGGPSVFLFPPKPKDTLMISRTPEVTCVVVDVSHEDPEVKFNWYVDGVEVHNAKT | 42.8.17 Fc LALA |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVEWFSGAQKLTNYKTTPPVLDSDGSFFLYSK | |
| | LTVDKSRWQQGNLFACEVMHEALHNHYTYKLLSLSPGK | |
| 96 | APEAAGGPSVFLFPPKPKDTLMISRTPEVTCVVVDVSHEDPEVKFNWYVDGVEVHNAKT | 42.2.19 Fc LALAPG |
| | KPREEQYNSTYRVVSVLTVLHQDWLNGKEYKCKVSNKALGAPIEKTISKAKGQPREPQV | |
| | YTLPPSRDELTKNQVSLTCLVKGFYPSDIAVEWFYDDSKLTNYKTTPPVLDSDGSFFLYSK | |
| | LTVDKSRWQPRGLFACEVMHEALHNHYTYKLLGESPGK | |
| 97 | APEAAGGPSVFLFPPKPKDTLMISRTPEVTCVVVDVSHEDPEVKFNWYVDGVEVHNAKT | 42.2.3-1H Fc |
| | KPREEQYNSTYRVVSVLTVLHQDWLNGKEYKCKVSNKALGAPIEKTISKAKGQPREPQV | LALAPG |
| | YTLPPSRDELTKNQVSLTCLVKGFYPSDIAVEWFYGNAKETNYKTTPPVLDSDGSFFLYS | |
| | KLTVDKSRWQQGNLFACEVMHEALHNHYTYKLLSLSPGK | |
| 98 | APEAAGGPSVFLFPPKPKDTLMISRTPEVTCVVVDVSHEDPEVKFNWYVDGVEVHNAKT | 42.8.196 Fc |
| | KPREEQYNSTYRVVSVLTVLHQDWLNGKEYKCKVSNKALGAPIEKTISKAKGQPREPQV | LALAPG |
| | YTLPPSRDELTKNQVSLTCLVKGFYPSDIAVEWFYEAQKLNNYKTTPPVLDSDGSFFLYS | |
| | KLTVDKSRWQQGNLFACEVMHEALHNHYTYKLLSLSPGK | |
| 99 | APEAAGGPSVFLFPPKPKDTLMISRTPEVTCVVVDVSHEDPEVKFNWYVDGVEVHNAKT | 42.8.80 Fc LALAPG |
| | KPREEQYNSTYRVVSVLTVLHQDWLNGKEYKCKVSNKALGAPIEKTISKAKGQPREPQV | |
| | YTLPPSRDELTKNQVSLTCLVKGFYPSDIAVEWFSEGSKETNYKTTPPVLDSDGSFFLYSK | |
| | LTVDKSRWQQGNLFACEVMHEALHNHYTYKLLSLSPGK | |
| 100 | APEAAGGPSVFLFPPKPKDTLMISRTPEVTCVVVDVSHEDPEVKFNWYVDGVEVHNAKT | 42.8.15 Fc LALAPG |
| | KPREEQYNSTYRVVSVLTVLHQDWLNGKEYKCKVSNKALGAPIEKTISKAKGQPREPQV | |
| | YTLPPSRDELTKNQVSLTCLVKGFYPSDIAVEWFSGAQKESNYKTTPPVLDSDGSFFLYSK | |
| | LTVDKSRWQQGNLFACEVMHEALHNHYTYKLLSLSPGK | |
| 101 | APEAAGGPSVFLFPPKPKDTLMISRTPEVTCVVVDVSHEDPEVKFNWYVDGVEVHNAKT | 42.8.17 Fc LALAPG |
| | KPREEQYNSTYRVVSVLTVLHQDWLNGKEYKCKVSNKALGAPIEKTISKAKGQPREPQV | |
| | YTLPPSRDELTKNQVSLTCLVKGFYPSDIAVEWFSGAQKLTNYKTTPPVLDSDGSFFLYSK | |
| | LTVDKSRWQQGNLFACEVMHEALHNHYTYKLLSLSPGK | |
| 102 | APEAAGGPSVFLFPPKPKDTLMISRTPEVTCVVVDVSHEDPEVKFNWYVDGVEVHNAKT | 42.2.19 Fc knob |
| | KPREEQYNSTYRVVSVLTVLHQDWLNGKEYKCKVSNKALPAPIEKTISKAKGQPREPQV | LALA |
| | YTLPPSRDELTKNQVSLWCLVKGFYPSDIAVEWFYDDSKLTNYKTTPPVLDSDGSFFLYS | |
| | KLTVDKSRWQPRGLFACEVMHEALHNHYTYKLLGESPGK | |
| 103 | APEAAGGPSVFLFPPKPKDTLMISRTPEVTCVVVDVSHEDPEVKFNWYVDGVEVHNAKT | 42.2.3-1H Fc knob |
| | KPREEQYNSTYRVVSVLTVLHQDWLNGKEYKCKVSNKALPAPIEKTISKAKGQPREPQV | LALA |
| | YTLPPSRDELTKNQVSLWCLVKGFYPSDIAVEWFYGNAKETNYKTTPPVLDSDGSFFLYS | |
| | KLTVDKSRWQQGNLFACEVMHEALHNHYTYKLLSLSPGK | |
| 104 | APEAAGGPSVFLFPPKPKDTLMISRTPEVTCVVVDVSHEDPEVKFNWYVDGVEVHNAKT | 42.8.196 Fc knob |
| | KPREEQYNSTYRVVSVLTVLHQDWLNGKEYKCKVSNKALPAPIEKTISKAKGQPREPQV | LALA |
| | YTLPPSRDELTKNQVSLWCLVKGFYPSDIAVEWFYEAQKLNNYKTTPPVLDSDGSFFLYS | |
| | KLTVDKSRWQQGNLFACEVMHEALHNHYTYKLLSLSPGK | |
| 105 | APEAAGGPSVFLFPPKPKDTLMISRTPEVTCVVVDVSHEDPEVKFNWYVDGVEVHNAKT | 42.8.80 Fc knob |
| | KPREEQYNSTYRVVSVLTVLHQDWLNGKEYKCKVSNKALPAPIEKTISKAKGQPREPQV | LALA |
| | YTLPPSRDELTKNQVSLWCLVKGFYPSDIAVEWFSEGSKETNYKTTPPVLDSDGSFFLYS | |
| | KLTVDKSRWQQGNLFACEVMHEALHNHYTYKLLSLSPGK | |
| 106 | APEAAGGPSVFLFPPKPKDTLMISRTPEVTCVVVDVSHEDPEVKFNWYVDGVEVHNAKT | 42.8.15 Fc knob |
| | KPREEQYNSTYRVVSVLTVLHQDWLNGKEYKCKVSNKALPAPIEKTISKAKGQPREPQV | LALA |
| | YTLPPSRDELTKNQVSLWCLVKGFYPSDIAVEWFSGAQKESNYKTTPPVLDSDGSFFLYS | |
| | KLTVDKSRWQQGNLFACEVMHEALHNHYTYKLLSLSPGK | |
| 107 | APEAAGGPSVFLFPPKPKDTLMISRTPEVTCVVVDVSHEDPEVKFNWYVDGVEVHNAKT | 42.8.17 Fc knob |
| | KPREEQYNSTYRVVSVLTVLHQDWLNGKEYKCKVSNKALPAPIEKTISKAKGQPREPQV | LALA |
| | YTLPPSRDELTKNQVSLWCLVKGFYPSDIAVEWFSGAQKLTNYKTTPPVLDSDGSFFLYS | |
| | KLTVDKSRWQQGNLFACEVMHEALHNHYTYKLLSLSPGK | |
| 108 | APEAAGGPSVFLFPPKPKDTLMISRTPEVTCVVVDVSHEDPEVKFNWYVDGVEVHNAKT | 42.2.19 Fc hole |
| | KPREEQYNSTYRVVSVLTVLHQDWLNGKEYKCKVSNKALPAPIEKTISKAKGQPREPQV | LALA |
| | YTLPPSRDELTKNQVSLSCAVKGFYPSDIAVEWFYDDSKLTNYKTTPPVLDSDGSFFLVS | |
| | KLTVDKSRWQPRGLFACEVMHEALHNHYTYKLLGESPGK | |
| 109 | APEAAGGPSVFLFPPKPKDTLMISRTPEVTCVVVDVSHEDPEVKFNWYVDGVEVHNAKT | 42.2.3-1H Fc hole |
| | KPREEQYNSTYRVVSVLTVLHQDWLNGKEYKCKVSNKALPAPIEKTISKAKGQPREPQV | LALA |
| | YTLPPSRDELTKNQVSLSCAVKGFYPSDIAVEWFYGNAKETNYKTTPPVLDSDGSFFLVS | |
| | KLTVDKSRWQQGNLFACEVMHEALHNHYTYKLLSLSPGK | |
| 110 | APEAAGGPSVFLFPPKPKDTLMISRTPEVTCVVVDVSHEDPEVKFNWYVDGVEVHNAKT | 42.8.196 Fc hole |
| | KPREEQYNSTYRVVSVLTVLHQDWLNGKEYKCKVSNKALPAPIEKTISKAKGQPREPQV | LALA |
| | YTLPPSRDELTKNQVSLSCAVKGFYPSDIAVEWFYEAQKLNNYKTTPPVLDSDGSFFLVS | |
| | KLTVDKSRWQQGNLFACEVMHEALHNHYTYKLLSLSPGK | |
| 111 | APEAAGGPSVFLFPPKPKDTLMISRTPEVTCVVVDVSHEDPEVKFNWYVDGVEVHNAKT | 42.8.80 Fc hole |
| | KPREEQYNSTYRVVSVLTVLHQDWLNGKEYKCKVSNKALPAPIEKTISKAKGQPREPQV | LALA |
| | YTLPPSRDELTKNQVSLSCAVKGFYPSDIAVEWFSEGSKETNYKTTPPVLDSDGSFFLVSK | |
| | LTVDKSRWQQGNLFACEVMHEALHNHYTYKLLSLSPGK | |
| 112 | APEAAGGPSVFLFPPKPKDTLMISRTPEVTCVVVDVSHEDPEVKFNWYVDGVEVHNAKT | 42.8.15 Fc hole |
| | KPREEQYNSTYRVVSVLTVLHQDWLNGKEYKCKVSNKALPAPIEKTISKAKGQPREPQV | LALA |
| | YTLPPSRDELTKNQVSLSCAVKGFYPSDIAVEWFSGAQKESNYKTTPPVLDSDGSFFLVSK | |
| | LTVDKSRWQQGNLFACEVMHEALHNHYTYKLLSLSPGK | |
| 113 | APEAAGGPSVFLFPPKPKDTLMISRTPEVTCVVVDVSHEDPEVKFNWYVDGVEVHNAKT | 42.8.17 Fc hole |
| | KPREEQYNSTYRVVSVLTVLHQDWLNGKEYKCKVSNKALPAPIEKTISKAKGQPREPQV | LALA |
| | YTLPPSRDELTKNQVSLSCAVKGFYPSDIAVEWFSGAQKLTNYKTTPPVLDSDGSFFLVS | |
| | KLTVDKSRWQQGNLFACEVMHEALHNHYTYKLLSLSPGK | |
| 114 | APEAAGGPSVFLFPPKPKDTLMISRTPEVTCVVVDVSHEDPEVKFNWYVDGVEVHNAKT | 42.2.19 Fc knob |
| | KPREEQYNSTYRVVSVLTVLHQDWLNGKEYKCKVSNKALGAPIEKTISKAKGQPREPQV | LALAPG |
| | YTLPPSRDELTKNQVSLWCLVKGFYPSDIAVEWFYDDSKLTNYKTTPPVLDSDGSFFLYS | |
| | KLTVDKSRWQPRGLFACEVMHEALHNHYTYKLLGESPGK | |
| 115 | APEAAGGPSVFLFPPKPKDTLMISRTPEVTCVVVDVSHEDPEVKFNWYVDGVEVHNAKT | 42.2.3-1H Fc knob |
| | KPREEQYNSTYRVVSVLTVLHQDWLNGKEYKCKVSNKALGAPIEKTISKAKGQPREPQV | LALAPG |
| | YTLPPSRDELTKNQVSLWCLVKGFYPSDIAVEWFYGNAKETNYKTTPPVLDSDGSFFLYS | |
| | KLTVDKSRWQQGNLFACEVMHEALHNHYTYKLLSLSPGK | |
| 116 | APEAAGGPSVFLFPPKPKDTLMISRTPEVTCVVVDVSHEDPEVKFNWYVDGVEVHNAKT | 42.8.196 Fc knob |
| | KPREEQYNSTYRVVSVLTVLHQDWLNGKEYKCKVSNKALGAPIEKTISKAKGQPREPQV | LALAPG |
| | YTLPPSRDELTKNQVSLWCLVKGFYPSDIAVEWFYEAQKLNNYKTTPPVLDSDGSFFLYS | |
| | KLTVDKSRWQQGNLFACEVMHEALHNHYTYKLLSLSPGK | |
| 117 | APEAAGGPSVFLFPPKPKDTLMISRTPEVTCVVVDVSHEDPEVKFNWYVDGVEVHNAKT | 42.8.80 Fc knob |
| | KPREEQYNSTYRVVSVLTVLHQDWLNGKEYKCKVSNKALGAPIEKTISKAKGQPREPQV | LALAPG |
| | YTLPPSRDELTKNQVSLWCLVKGFYPSDIAVEWFSEGSKETNYKTTPPVLDSDGSFFLYS | |
| | KLTVDKSRWQQGNLFACEVMHEALHNHYTYKLLSLSPGK | |
| 118 | APEAAGGPSVFLFPPKPKDTLMISRTPEVTCVVVDVSHEDPEVKFNWYVDGVEVHNAKT | 42.8.15 Fc knob |
| | KPREEQYNSTYRVVSVLTVLHQDWLNGKEYKCKVSNKALGAPIEKTISKAKGQPREPQV | LALAPG |
| | YTLPPSRDELTKNQVSLWCLVKGFYPSDIAVEWFSGAQKESNYKTTPPVLDSDGSFFLYS | |
| | KLTVDKSRWQQGNLFACEVMHEALHNHYTYKLLSLSPGK | |
| 119 | APEAAGGPSVFLFPPKPKDTLMISRTPEVTCVVVDVSHEDPEVKFNWYVDGVEVHNAKT | 42.8.17 Fc knob |
| | KPREEQYNSTYRVVSVLTVLHQDWLNGKEYKCKVSNKALGAPIEKTISKAKGQPREPQV | LALAPG |
| | YTLPPSRDELTKNQVSLWCLVKGFYPSDIAVEWFSGAQKLTNYKTTPPVLDSDGSFFLYS | |
| | KLTVDKSRWQQGNLFACEVMHEALHNHYTYKLLSLSPGK | |
| 120 | APEAAGGPSVFLFPPKPKDTLMISRTPEVTCVVVDVSHEDPEVKFNWYVDGVEVHNAKT | 42.2.19 Fc hole |
| | KPREEQYNSTYRVVSVLTVLHQDWLNGKEYKCKVSNKALGAPIEKTISKAKGQPREPQV | LALAPG |
| | YTLPPSRDELTKNQVSLTSLSCLVCAVKGFYPSDIAVEWFYDDSKLTNYKTTPPVLDSDG | |
| | SFFLYSLVSKLTVDKSRWQPRGLFACEVMHEALHNHYTYKLLGESPGK | |
| 121 | APEAAGGPSVFLFPPKPKDTLMISRTPEVTCVVVDVSHEDPEVKFNWYVDGVEVHNAKT | 42.2.3-1H Fc hole |
| | KPREEQYNSTYRVVSVLTVLHQDWLNGKEYKCKVSNKALGAPIEKTISKAKGQPREPQV | LALAPG |
| | YTLPPSRDELTKNQVSLTSLSCLVCAVKGFYPSDIAVEWFYGNAKETNYKTTPPVLDSDG | |
| | SFFLYSLVSKLTVDKSRWQQGNLFACEVMHEALHNHYTYKLLSLSPGK | |
| 122 | APEAAGGPSVFLFPPKPKDTLMISRTPEVTCVVVDVSHEDPEVKFNWYVDGVEVHNAKT | 42.8.196 Fc hole |
| | KPREEQYNSTYRVVSVLTVLHQDWLNGKEYKCKVSNKALGAPIEKTISKAKGQPREPQV | LALAPG |
| | YTLPPSRDELTKNQVSLTSLSCLVCAVKGFYPSDIAVEWFYEAQKLNNYKTTPPVLDSDG | |
| | SFFLYSLVSKLTVDKSRWQQGNLFACEVMHEALHNHYTYKLLSLSPGK | |
| 123 | APEAAGGPSVFLFPPKPKDTLMISRTPEVTCVVVDVSHEDPEVKFNWYVDGVEVHNAKT | 42.8.80 Fc hole |
| | KPREEQYNSTYRVVSVLTVLHQDWLNGKEYKCKVSNKALGAPIEKTISKAKGQPREPQV | LALAPG |
| | YTLPPSRDELTKNQVSLTSLSCLVCAVKGFYPSDIAVEWFSEGSKETNYKTTPPVLDSDGS | |
| | FFLYSLVSKLTVDKSRWQQGNLFACEVMHEALHNHYTYKLLSLSPGK | |
| 124 | APEAAGGPSVFLFPPKPKDTLMISRTPEVTCVVVDVSHEDPEVKFNWYVDGVEVHNAKT | 42.8.15 Fc hole |
| | KPREEQYNSTYRVVSVLTVLHQDWLNGKEYKCKVSNKALGAPIEKTISKAKGQPREPQV | LALAPG |
| | YTLPPSRDELTKNQVSLTSLSCLVCAVKGFYPSDIAVEWFSGAQKESNYKTTPPVLDSDG | |
| | SFFLYSLVSKLTVDKSRWQQGNLFACEVMHEALHNHYTYKLLSLSPGK | |
| 125 | APEAAGGPSVFLFPPKPKDTLMISRTPEVTCVVVDVSHEDPEVKFNWYVDGVEVHNAKT | 42.8.17 Fc hole |
| | KPREEQYNSTYRVVSVLTVLHQDWLNGKEYKCKVSNKALGAPIEKTISKAKGQPREPQV | LALAPG |
| | YTLPPSRDELTKNQVSLSTCALVKGFYPSDIAVEWFSGAQKLTNYKTTPPVLDSDGSFFLV | |
| | YSKLTVDKSRWQQGNLFACEVMHEALHNHYTYKLLSLSPGK | |
| 126 | APELLGGPSVFLFPPKPKDTLMISRTPEVTCVVVDVSHEDPEVKFNWYVDGVEVHNAKT | CH3C.35.21 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIAVWWESYGTEWSSYKTTPPVLDSDGSFFLYS | |
| | KLTVTKEEWQQGFVFSCSVMHEALHNHYTQKSLSLSPGK | |
| 127 | MMDQARSAFSNLFGGEPLSYTRFSLARQVDGDNSHVEMKLAVDEEENADNNTKANVTK | Human transferrin |
| | PKRCSGSICYGTIAVIVFFLIGFMIGYLGYCKGVEPKTECERLAGTESPVREEPGEDFPAAR | receptor protein 1 |
| | RLYWDDLKRKLSEKLDSTDFTGTIKLLNENSYVPREAGSQKDENLALYVENQFREFKLSK | (TFR1 |
| | VWRDQHFVKIQVKDSAQNSVIIVDKNGRLVYLVENPGGYVAYSKAATVTGKLVHANFG | |
| | TKKDFEDLYTPVNGSIVIVRAGKITFAEKVANAESLNAIGVLIYMDQTKFPIVNAELSFFG | |
| | HAHLGTGDPYTPGFPSFNHTQFPPSRSSGLPNIPVQTISRAAAEKLFGNMEGDCPSDWKTD | |
| | STCRMVTSESKNVKLTVSNVLKEIKILNIFGVIKGFVEPDHYVVVGAQRDAWGPGAAKS | |
| | GVGTALLLKLAQMFSDMVLKDGFQPSRSIIFASWSAGDFGSVGATEWLEGYLSSLHLKA | |
| | FTYINLDKAVLGTSNFKVSASPLLYTLIEKTMQNVKHPVTGQFLYQDSNWASKVEKLTLD | |
| | NAAFPFLAYSGIPAVSFCFCEDTDYPYLGTTMDTYKELIERIPELNKVARAAAEVAGQFVI | |
| | KLTHDVELNLDYERYNSQLLSFVRDLNQYRADIKEMGLSLQWLYSARGDFFRATSRLTT | |
| | DFGNAEKTDRFVMKKLNDRVMRVEYHFLSPYVSPKESPFRHVFWGSGSHTLPALLENLK | |
| | LRKQNNGAFNETLFRNQLALATWTIQGAANALSGDVWDIDNEF | |
| 128 | MMDQARSAFSNLFGGEPLSYTRFSLARQVDGDNSHVEMKLGVDEEENTDNNTKANGTK | Cyno TfR |
| | PKRCGGNICYGTIAVIIFFLIGFMIGYLGYCKGVEPKTECERLAGTESPAREEPEEDFPAAP | |
| | RLYWDDLKRKLSEKLDTTDFTSTIKLLNENLYVPREAGSQKDENLALYIENQFREFKLSK | |
| | VWRDQHFVKIQVKDSAQNSVIIVDKNGGLVYLVENPGGYVAYSKAATVTGKLVHANFG | |
| | TKKDFEDLDSPVNGSIVIVRAGKITFAEKVANAESLNAIGVLIYMDQTKFPIVKADLSFFG | |
| | HAHLGTGDPYTPGFPSFNHTQFPPSQSSGLPNIPVQTISRAAAEKLFGNMEGDCPSDWKTD | |
| | STCKMVTSENKSVKLTVSNVLKETKILNIFGVIKGFVEPDHYVVVGAQRDAWGPGAAKS | |
| | SVGTALLLKLAQMFSDMVLKDGFQPSRSIIFASWSAGDFGSVGATEWLEGYLSSLHLKAF | |
| | TYINLDKAVLGTSNFKVSASPLLYTLIEKTMQDVKHPVTGRSLYQDSNWASKVEKLTLD | |
| | NAAFPFLAYSGIPAVSFCFCEDTDYPYLGTTMDTYKELVERIPELNKVARAAAEVAGQFV | |
| | IKLTHDTELNLDYERYNSQLLLFLRDLNQYRADVKEMGLSLQWLYSARGDFFRATSRLT | |
| | TDFRNAEKRDKFVMKKLNDRVMRVEYYFLSPYVSPKESPFRHVFWGSGSHTLSALLESL | |
| | KLRRQNNSAFNETLFRNQLALATWTIQGAANALSGDVWDIDNEF | |
| 129 | NSVIIVDKNGRLVYLVENPGGYVAYSKAATVTGKLVHANFGTKKDFEDLYTPVNGSIVIV | Human TfR apical |
| | RAGKITFAEKVANAESLNAIGVLIYMDQTKFPIVNAELSFFGHAHLGTGDPYTPGFPSFNH | domain |
| | TQFPPSRSSGLPNIPVQTISRAAAEKLFGNMEGDCPSDWKTDSTCRMVTSESKNVKLTVS | |
| 130 | NSVIIVDKNGGLVYLVENPGGYVAYSKAATVTGKLVHANFGTKKDFEDLDSPVNGSIVIV | Cynomolgus TfR |
| | RAGKITFAEKVANAESLNAIGVLIYMDQTKFPIVKADLSFFGHAHLGTGDPYTPGFPSFNH | apical domain |
| | TQFPPSQSSGLPNIPVQTISRAAAEKLFGNMEGDCPSDWKTDSTCKMVTSENKSVKLTVS | |
| 131 | SSGLPNIPVQTISRAAAEKLFGNMEGDCPSDWKTDSTCRMVTSESKNVKLTVSNDSAQNS | Loop-truncated |
| | VIIVDKNGRLVYLVENPGGYVAYSKAATVTGKLVHANFGTKKDFEDLYTPVNGSIVIVR | human TfR apical |
| | AGKITFAEKVANAESLNAIGVLIYMDQTKFPIVNAELSGP | domain displayed on |
| | | phage |
| 132 | SSGLPNIPVQTISRAAAEKLFGNMEGDCPSDWKTDSTCKMVTSENKSVKLTVSNDSAQNS | Loop-truncated |
| | VIIVDKNGGLVYLVENPGGYVAYSKAATVTGKLVHANFGTKKDFEDLDSPVNGSIVIVR | cynomolgus TfR |
| | AGKITFAEKVANAESLNAIGVLIYMDQTKFPIVKADLSGP | apical domain |
| | | displayed on phage |
| 133 | MGWSCIILFLVATATGAYAGTSSGLPNIPVQTISRAAAEKLFGNMEGDCPSDWKTDSTCR | His-tagged |
| | MVTSESKNVKLTVSNDSAQNSVIIVDKNGRLVYLVENPGGYVAYSKAATVTGKLVHANF | permutated TfR |
| | GTKKDFEDLYTPVNGSIVIVRAGKITFAEKVANAESLNAIGVLIYMDQTKFPIVNAELSAS | apical domain |
| | HHHHHH | |
| 134 | MSQDTEVDMKEVELNELEPEKQPMNAASGAAMSLAGAEKNGLVKIKVAEDEAEAAAA | Uniprot P08195- |
| | AKFTGLSKEELLKVAGSPGWVRTRWALLLLFWLGWLGMLAGAVVIIVRAPRCRELPAQ | 2\|4F2_HUMAN |
| | KWWHTGALYRIGDLQAFQGHGAGNLAGLKGRLDYLSSLKVKGLVLGPIHKNQKDDVA | Isoform 2 of 4F2 |
| | QTDLLQIDPNFGSKEDFDSLLQSAKKKSIRVILDLTPNYRGENSWFSTQVDTVATKVKDA | cell-surface antigen |
| | LEFWLQAGVDGFQVRDIENLKDASSFLAEWQNITKGFSEDRLLIAGTNSSDLQQILSLLES | heavy chain |
| | NKDLLLTSSYLSDSGSTGEHTKSLVTQYLNATGNRWCSWSLSQARLLTSFLPAQLLRLYQ | |
| | LMLFTLPGTPVFSYGDEIGLDAAALPGQPMEAPVMLWDESSFPDIPGAVSANMTVKGQS | |
| | EDPGSLLSLFRRLSDQRSKERSLLHGDFHAFSAGPGLFSYIRHWDQNERFLVVLNFGDVG | |
| | LSAGLQASDLPASASLPAKADLLLSTQPGREEGSPLELERLKLEPHEGLLLRFPYAA | |
| 135 | CRELPAQKWWHTGALYRIGDLQAFQGHGAGNLAGLKGRLDYLSSLKVKGLVLGPIHKN | hCD98hc ECD: |
| | QKDDVAQTDLLQIDPNFGSKEDFDSLLQSAKKKSIRVILDLTPNYRGENSWFSTQVDTVA | positions 109-520 of |
| | TKVKDALEFWLQAGVDGFQVRDIENLKDASSFLAEWQNITKGFSEDRLLIAGTNSSDLQQ | Uniprot P08195- |
| | ILSLLESNKDLLLTSSYLSDSGSTGEHTKSLVTQYLNATGNRWCSWSLSQARLLTSFLPAQ | 2\|4F2_HUMAN |
| | LLRLYQLMLFTLPGTPVFSYGDEIGLDAAALPGQPMEAPVMLWDESSFPDIPGAVSANMT | Isoform 2 of 4F2 |
| | VKGQSEDPGSLLSLFRRLSDQRSKERSLLHGDFHAFSAGPGLFSYIRHWDQNERFLVVLN | cell-surface antigen |
| | FGDVGLSAGLQASDLPASASLPAKADLLLSTQPGREEGSPLELERLKLEPHEGLLLRFPYA | heavy chain |
| | A | |
| 136 | GSELPAQKWWHTGALYRIGDLQAFQGHGAGNLAGLKGRLDYLSSLKVKGLVLGPIHKN | hCD98hc ECD for |
| | QKDDVAQTDLLQIDPNFGSKEDFDSLLQSAKKKSIRVILDLTPNYRGENSWFSTQVDTVA | crystal structures: |
| | TKVKDALEFWLQAGVDGFQVRDIENLKDASSFLAEWQNITKGFSEDRLLIAGTNSSDLQQ | positions 109-520 of |
| | ILSLLESNKDLLLTSSYLSDSGSTGEHTKSLVTQYLNATGNRWCSWSLSQARLLTSFLPAQ | Uniprot P08195- |
| | LLRLYQLMLFTLPGTPVFSYGDEIGLDAAALPGQPMEAPVMLWDESSFPDIPGAVSANMT | 2\|4F2_HUMAN |
| | VKGQSEDPGSLLSLFRRLSDQRSKERSLLHGDFHAFSAGPGLFSYIRHWDQNERFLVVLN | Isoform 2 of 4F2 |
| | FGDVGLSAGLQASDLPASASLPAKADLLLSTQPGREEGSPLELERLKLEPHEGLLLRFPYA | cell-surface antigen |
| | A | heavy chain with G |
| | | at position 109 and |
| | | S at position 110 |
| | | in crystal structures |
| 137 | APELLGGPSVFLFPPKPKDTLMISRTPEVTCVVVDVSHEDPEVKFNWYVDGVEVHNAKT | Clone 1-321 |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIHVEWGSLVQPENNYKTTPPVLDSDGSFFLYSK | |
| | LTVDKSRWQQGNIFPCIVMHEALHNHYTIKTLSLSPGK | |
| 138 | APELLGGPSVFLFPPKPKDTLMISRTPEVTCVVVDVSHEDPEVKFNWYVDGVEVHNAKT | Clone 1-321_M428L |
| | KPREEQYNSTYRVVSVLTVLHQDWLNGKEYKCKVSNKALPAPIEKTISKAKGQPREPQV | |
| | YTLPPSRDELTKNQVSLTCLVKGFYPSDIHVEWGSLVQPENNYKTTPPVLDSDGSFFLYSK | |
| | LTVDKSRWQQGNIFPCIVLHEALHNHYTIKTLSLSPGK | |

1-8. (canceled)

9. A polypeptide comprising a modified CH3 domain that specifically binds to a CD98hc protein, wherein the modified CH3 domain comprises a sequence having at least 95% sequence identity to amino acids 111-217 of the sequence of any one of SEQ ID NOS:28-43, wherein the modified CH3 domain comprises at least eleven, twelve, thirteen, fourteen, or fifteen substitutions in a set of amino acid positions consisting of a L at position 380, a N or S at position 382, a R, H, or Q at position 384, a F or Y at position 385, a V, L, I, F, Y, or E at position 386, a L at position 387, a E, Q, or A at position 421, a I, T, or P at position 422, an A at position 424, a N at position 426, a Y or W at position 428, a R or W at position 436, a F or W at position 438, a N at position 440, and an A, Q, K, R, H, or M at position 442, according to EU numbering.

10. (canceled)

11. The polypeptide of claim 9, wherein:
(a) the modified CH3 domain comprises a L at position 380, a N at position 382, a H at position 384, a Y at position 385, a E at position 386, a L at position 387, an I at position 422, an A at position 424, a N at position 426, a Y at position 428, a F at position 438, a N at position 440, and an A at position 442; or
(b) the modified CH3 domain comprises a L at position 380, a N at position 382, a R at position 384, a F at position 385, a V at position 386, a L at position 387, an E at position 421, an I at position 422, an A at position 424, a N at position 426, a Y at position 428, a F at position 438, a N at position 440, and an A at position 442; or
(c) the modified CH3 domain comprises a L at position 380, a N at position 382, a H at position 384, a Y at position 385, a E at position 386, a L at position 387, an A at position 424, a N at position 426, a Y at position 428, a F at position 438, a N at position 440, and an A at position 442; or
(d) the modified CH3 domain comprises a L at position 380, a N at position 382, a R at position 384, a F at position 385, a V at position 386, a L at position 387, an A at position 424, a N at position 426, a Y at position 428, a F at position 438, a N at position 440, and an A at position 442;
(e) the modified CH3 domain comprises a L at position 380, a N at position 382, a R at position 384, a F at position 385, a V at position 386, a L at position 387, an I at position 422, an A at position 424, a N at position 426, a Y at position 428, a F at position 438, a N at position 440, and a R at position 442; or
(f) the modified CH3 domain comprises a L at position 380, a N at position 382, a R at position 384, a F at position 385, a V at position 386, a L at position 387, an I at position 422, an A at position 424, a N at position 426, a Y at position 428, a F at position 438, a N at position 440, and a H at position 442; or
(g) the modified CH3 domain comprises a L at position 380, a N at position 382, a H at position 384, a Y at position 385, a E at position 386, a L at position 387, an E at position 421, an A at position 424, a N at position 426, a Y at position 428, a F at position 438, a N at position 440, and an A at position 442; or
(h) the modified CH3 domain comprises a L at position 380, a N at position 382, a Q at position 384, a Y at position 385, a E at position 386, a L at position 387, an E at position 421, an I at position 422, an A at position 424, a N at position 426, a Y at position 428, a F at position 438, a N at position 440, and an A at position 442; or
(i) the modified CH3 domain comprises a L at position 380, a N at position 382, a R at position 384, a F at position 385, a V at position 386, a L at position 387, an I at position 422, an A at position 424, a N at position 426, a Y at position 428, a F at position 438, a N at position 440, and an A at position 442; or
(i) the modified CH3 domain comprises a L at position 380, a N at position 382, a Q at position 384, a Y at position 385, a E at position 386, a L at position 387, an A at position 424, a N at position 426, a Y at position 428, a F at position 438, a N at position 440, and an A at position 442; or
(k) the modified CH3 domain comprises a L at position 380, a N at position 382, a R at position 384, a F at position 385, a V at position 386, a L at position 387, an I at position 422, an A at position 424, a N at position 426, a Y at position 428, a R at position 436, a F at position 438, a N at position 440, and a R at position 442; or
(1) the modified CH3 domain comprises a L at position 380, a N at position 382, a R at position 384, a F at position 385, a V at position 386, a L at position 387, an I at position 422, an A at position 424, a N at position 426, a Y at position 428, a F at position 438, a N at position 440, and a K at position 442; or
(m) the modified CH3 domain comprises a L at position 380, a N at position 382, a R at position 384, a F at position 385, a V at position 386, a L at position 387, an I at position 422, an A at position 424, a N at position 426, a Y at position 428, a W at position 436, a F at position 438, a N at position 440, and a R at position 442; or
(n) the modified CH3 domain comprises a L at position 380, a N at position 382, a Q at position 384, a Y at position 385, a L at position 386, a L at position 387, an E at position 421, an I at position 422, an A at position 424, a N at position 426, a Y at position 428, a F at position 438, a N at position 440, and an A at position 442; or
(o) the modified CH3 domain comprises a L at position 380, a S at position 382, a R at position 384, a Y at position 385, a V at position 386, a L at position 387, an A at position 424, a N at position 426, a Y at position 428, a F at position 438, a N at position 440, and an A at position 442; or
(p) the modified CH3 domain comprises a L at position 380, a N at position 382, a R at position 384, a F at position 385, a V at position 386, a L at position 387, an E at position 421, an A at position 424, a N at position 426, a Y at position 428, a F at position 438, a N at position 440, and an A at position 442; or
(q) the modified CH3 domain comprises a L at position 380, a N at position 382, a R at position 384, a F at position 385, a V at position 386, a L at position 387, an E at position 421, an I at position 422, an A at position 424, a N at position 426, a Y at position 428, a F at position 438, and a N at position 440; or
(r) the modified CH3 domain comprises a L at position 380, a N at position 382, a Q at position 384, a F at position 385, a H at position 386, a L at position 387, an I at position 422, an A at position 424, a N at position 426, a Y at position 428, a F at position 438, a N at position 440, and a L at position 442; or
(s) the modified CH3 domain comprises a L at position 380, a N at position 382, a R at position 384, a F at position 385, a V at position 386, a L at position 387, an T at position 422, an A at position 424, a N at position 426, a Y at position 428, a F at position 438, a N at position 440, and an A at position 442.

12. The polypeptide of claim 11, wherein the modified CH3 domain comprises a sequence selected from any one of SEQ ID NOS:38, 29, 31, 32, 35, 36, 28, 30, 37, 41, 42, 43, 33, 34, 39, and 40.

13-43. (canceled)

44. The polypeptide of claim 9, wherein the polypeptide:
(a) binds human CD98hc with an affinity of 15 nM to 5 μM; and/or
(b) has cyno cross-reactivity; and/or
(c) binds to cyno CD98hc with an affinity of 80 nM to 5 μM.

45-50. (canceled)

51. A polypeptide comprising a modified CH3 domain that specifically binds to a CD98hc protein, wherein the modified CH3 domain comprises a sequence having at least 95% sequence identity to amino acids 111-217 of the sequence of any one of SEQ ID NOS:44-45, wherein the modified CH3 domain comprises at least eight, nine, ten, eleven, twelve, thirteen, fourteen, or fifteen substitutions in a set of amino acid positions consisting of a R at position 382, a T at position 383, a Y at position 384, a K at position 385, a P at position 386, a Y at position 387, a T at position 389, a D at position 421, an I at position 422, a V at position 424, a D at position 426, a L at position 428, a F at position 436, a I at position 438, and a K at position 440, according to EU numbering.

52. (canceled)

53. The polypeptide of claim 51, wherein:
(a) the modified CH3 domain comprises a R at position 382, a T at position 383, a Y at position 384, a K at position 385, a P at position 386, a Y at position 387, a T at position 389, a D at position 421, an I at position 422, a V at position 424, a D at position 426, a F at position 436, a I at position 438, and a K at position 440; or
(b) wherein the modified CH3 domain comprises a R at position 382, a T at position 383, a Y at position 384, a K at position 385, a P at position 386, a Y at position 387, a T at position 389, a D at position 421, an I at position 422, a V at position 424, a D at position 426, a L at position 428, a F at position 436, a I at position 438, and a K at position 440.

54. The polypeptide of claim 53, wherein the modified CH3 domain comprises SEQ ID NO:44 or SEQ ID NO:45.

55-140. (canceled)

141. The polypeptide of claim 9, wherein the modified CH3 domain further comprises at least one modification that promotes heterodimerization; and/or wherein the polypeptide further comprises L at position 428 and S at position 434.

142. The polypeptide of claim 141, wherein the at least one modification that promotes heterodimerization comprises a T366W substitution, according to EU numbering; or
wherein the at least one modification that promotes heterodimerization comprises T366S, L368A, and Y407V substitutions, according to EU numbering.

143. (canceled)

144. (canceled)

145. The polypeptide of claim 9, wherein the polypeptide further comprises a modified CH2 domain comprising modifications that reduce effector function, wherein the modifications that reduce effector function comprise A1a at position 234 and A1a at position 235 and/or Gly or Ser at position 329, according to EU numbering.

146-151. (canceled)

152. The polypeptide of claim 9, wherein:
(a) the polypeptide is part of a dimer; and/or
(b) the polypeptide is further joined to a Fab; and/or
(c) the C-terminal lysine of the polypeptide is absent.

153. (canceled)

154. (canceled)

155. The polypeptide of claim 152, wherein the polypeptide is a first polypeptide of a dimer such that the dimer is monovalent for CD98hc binding; or wherein the polypeptide is a first polypeptide of a dimer such that the dimer is bivalent for CD98hc binding.

156-159. (canceled)

160. A polynucleotide comprising a nucleic acid sequence encoding the polypeptide of claim 9.

161. A vector or host cell comprising the polynucleotide of claim 160.

162. (canceled)

163. A method for producing a polypeptide comprising a modified constant domain or modified CH3 domain, comprising culturing a host cell under conditions in which the polypeptide encoded by the polynucleotide of claim 160 is expressed.

164. A pharmaceutical composition comprising the polypeptide of claim 9 and a pharmaceutically acceptable carrier.

165. A method of transcytosis across an endothelium, comprising contacting the endothelium with a composition comprising the polypeptide of claim 9 fused to a therapeutic agent.

166. (canceled)

167. (canceled)

168. The method of claim 165, wherein the endothelium is the BBB.

169-178. (canceled)

179. The method of claim 165, further comprising delivering the therapeutic agent to a biological target in the brain.

180-203. (canceled)

204. A method of increasing brain exposure to a therapeutic agent in a subject relative to a reference molecule, the method comprising administering to the subject a monovalent molecule that binds to CD98hc with a binding affinity from about 20 nM to about 550 nM or a bivalent molecule that binds to CD98hc with a binding affinity from about 275 nM to about 2100 nM, wherein the molecule is linked to the therapeutic agent, and wherein the reference molecule comprises the therapeutic agent but not a CD98hc binding moiety.

205-287. (canceled)


SEQUENCE LISTING (142 sequences)

SEQ ID NO: 1 - protein, 217 aa, Homo sapiens
  REGION 1..217: note = Description of sequence: Wild-type human Fc sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 110 aa, Homo sapiens
  REGION 1..110: note = Description of sequence: CH2 domain sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 3 - protein, 107 aa, Homo sapiens
  REGION 1..107: note = Description of sequence: CH3 domain sequence
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 15 aa, Homo sapiens
  REGION 1..15: note = Description of sequence: human IgG1 hinge
EPKSCDKTHTCPPCP

SEQ ID NO: 5 - protein, 217 aa, synthetic construct
  REGION 1..217: note = Description of sequence: Fc sequence with knob mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 217 aa, synthetic construct
  REGION 1..217: note = Description of sequence: Fc sequence with hole mutations
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 7 - protein, 217 aa, synthetic construct
  REGION 1..217: note = Description of sequence: Fc sequence with LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 217 aa, synthetic construct
  REGION 1..217: note = Description of sequence: Fc sequence with knob and LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 217 aa, synthetic construct
  REGION 1..217: note = Description of sequence: Fc sequence with hole and LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 217 aa, synthetic construct
  REGION 1..217: note = Description of sequence: Fc sequence with LALA and PG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 217 aa, synthetic construct
  REGION 1..217: note = Description of sequence: Fc sequence with knob and LALA and PG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 217 aa, synthetic construct
  REGION 1..217: note = Description of sequence: Fc sequence with hole and LALA and PG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 217 aa, synthetic construct
  REGION 1..217: note = Description of sequence: Fc sequence with LALA and PS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 217 aa, synthetic construct
  REGION 1..217: note = Description of sequence: Fc sequence with knob and LALA and PS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 217 aa, synthetic construct
  REGION 1..217: note = Description of sequence: Fc sequence with hole and LALA and PS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 217 aa, synthetic construct
  REGION 1..217: note = Description of sequence: Fc sequence with LS
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 17 - protein, 217 aa, synthetic construct
  REGION 1..217: note = Description of sequence: Fc sequence with knob and LS
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 18 - protein, 217 aa, synthetic construct
  REGION 1..217: note = Description of sequence: Fc sequence with hole and LS
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 19 - protein, 217 aa, synthetic construct
  REGION 1..217: note = Description of sequence: Fc sequence with LALA and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 20 - protein, 217 aa, synthetic construct
  REGION 1..217: note = Description of sequence: Fc sequence with knob and LALA and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 21 - protein, 217 aa, synthetic construct
  REGION 1..217: note = Description of sequence: Fc sequence with hole and LALA and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 22 - protein, 217 aa, synthetic construct
  REGION 1..217: note = Description of sequence: Fc sequence with LALA and PG and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 23 - protein, 217 aa, synthetic construct
  REGION 1..217: note = Description of sequence: Fc sequence with knob and LALA and PG and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 24 - protein, 217 aa, synthetic construct
  REGION 1..217: note = Description of sequence: Fc sequence with hole and LALA and PG and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 25 - protein, 217 aa, synthetic construct
  REGION 1..217: note = Description of sequence: Fc sequence with LALA and PS and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 26 - protein, 217 aa, synthetic construct
  REGION 1..217: note = Description of sequence: Fc sequence with knob and LALA and PS and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 27 - protein, 217 aa, synthetic construct
  REGION 1..217: note = Description of sequence: Fc sequence with hole and LALA and PS and LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 28 - protein, 217 aa, synthetic construct
  REGION 1..217: note = Description of sequence: Clone LLB2-10-6
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGNIFACNVYHEALHNHYTFKNLALSPGK

SEQ ID NO: 29 - protein, 217 aa, synthetic construct
  REGION 1..217: note = Description of sequence: Clone LLB2-10-8
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLALSPGK

SEQ ID NO: 30 - protein, 217 aa, synthetic construct
  REGION 1..217: note = Description of sequence: Clone LLB2-10-8-d18
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSQYELENNYKTTPPVLDSDGSFFLYSKLTVDKSRWQQGNVFACNVYHEALHNHYTFKNLALSPGK

SEQ ID NO: 31 - protein, 217 aa, synthetic construct
  REGION 1..217: note = Description of sequence: Clone LLB2-10-8-d12
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSHYELENNYKTTPPVLDSDGSFFLYSKLTVDKSRWQQGNVFACNVYHEALHNHYTFKNLALSPGK

SEQ ID NO: 32 - protein, 217 aa, synthetic construct
  REGION 1..217: note = Description of sequence: Clone LLB2-10-8-d6
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGNVFACNVYHEALHNHYTFKNLALSPGK

SEQ ID NO: 33 - protein, 217 aa, synthetic construct
  REGION 1..217: note = Description of sequence: Clone LLB2-10-8-d3
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEVFACNVYHEALHNHYTFKNLALSPGK

SEQ ID NO: 34 - protein, 217 aa, synthetic construct
  REGION 1..217: note = Description of sequence: Clone LLB2-10-8-d1
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLSLSPGK

SEQ ID NO: 35 - protein, 217 aa, synthetic construct
  REGION 1..217: note = Description of sequence: Clone LLB2.10.8.10.3
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLRLSPGK

SEQ ID NO: 36 - protein, 217 aa, synthetic construct
  REGION 1..217: note = Description of sequence: Clone LLB2.10.8.10.8
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLHLSPGK

SEQ ID NO: 37 - protein, 217 aa, synthetic construct
  REGION 1..217: note = Description of sequence: Clone LLB2.10.8.14.3
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHRTFKNLRLSPGK

SEQ ID NO: 38 - protein, 217 aa, synthetic construct
  REGION 1..217: note = Description of sequence: Clone LLB2.10.8.12.5
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSHYELENNYKTTPPVLDSDGSFFLYSKLTVDKSRWQQGEIFACNVYHEALHNHYTFKNLALSPGK

SEQ ID NO: 39 - protein, 217 aa, synthetic construct
  REGION 1..217: note = Description of sequence: Clone LLB2-37
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSQFHLENNYKTTPPVLDSDGSFFLYSKLTVDKSRWQQGNIFACNVYHEALHNHYTFKNLLLSPGK

SEQ ID NO: 40 - protein, 217 aa, synthetic construct
  REGION 1..217: note = Description of sequence: Clone LLB2.10.8.9.11.N
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGNTFACNVYHEALHNHYTFKNLALSPGK

SEQ ID NO: 41 - protein, 217 aa, synthetic construct
  REGION 1..217: note = Description of sequence: Clone LLB2.10.8.10.1
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLKLSPGK

SEQ ID NO: 42 - protein, 217 aa, synthetic construct
  REGION 1..217: note = Description of sequence: Clone LLB2.10.8.4.12
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHWTFKNLRLSPGK

SEQ ID NO: 43 - protein, 217 aa, synthetic construct
  REGION 1..217: note = Description of sequence: Clone LLB2.10.8.2.1
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSQYLLENNYKTTPPVLDSDGSFFLYSKLTVDKSRWQQGEIFACNVYHEALHNHYTFKNLALSPGK

SEQ ID NO: 44 - protein, 217 aa, synthetic construct
  REGION 1..217: note = Description of sequence: Clone LLB1-3-16-2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWRTYKPYETNYKTTPPVLDSDGSFFLYSKLTVDKSRWQQGDIFVCDVMHEALHNHFTIKKLSLSPGK

SEQ ID NO: 45 - protein, 217 aa, synthetic construct
  REGION 1..217: note = Description of sequence: Clone LLB1-3-16
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWRTYKPYETNYKTTPPVLDSDGSFFLYSKLTVDKSRWQQGDIFVCDVLHEALHNHFTIKKLSLSPGK

SEQ ID NO: 46 - protein, 8 aa, synthetic construct
  REGION 1..8: note = Description of sequence: LLB2 Consensus 380-387
  VARIANT 2: note = X can be any naturally occurring amino acid
  VARIANT 4: note = X can be either R or H
  VARIANT 5: note = F can be also be Y
  VARIANT 6: note = X can be V, L, I, F, Y, or E
  VARIANT 7: note = X can be any naturally occurring amino acid
LXNXFXXL

SEQ ID NO: 47 - protein, 8 aa, synthetic construct
  REGION 1..8: note = Description of sequence: LLB2 Consensus 421-428
  VARIANT 1: note = E can also be replaced with N, Q or A
  VARIANT 2: note = X can be I, V, T, or P
  VARIANT 3: note = X can be any naturally occurring amino acid
  VARIANT 5: note = X can be any naturally occurring amino acid
  VARIANT 6: note = S can also be N
  VARIANT 7: note = X can be any naturally occurring amino acid
  VARIANT 7: note = Y and also be W
EXXAXSXY

SEQ ID NO: 48 - protein, 7 aa, synthetic construct
  REGION 1..7: note = Description of sequence: LLB2 Consensus 436-442
  VARIANT 1: note = Y can also be either R or W
  VARIANT 2: note = X can be any naturally occurring amino acid
  VARIANT 3: note = F can also be W
  VARIANT 4: note = X can be any naturally occurring amino acid
  VARIANT 6: note = X can be any naturally occurring amino acid
  VARIANT 7: note = A can also be Q, K, R, H, M, or S
YXFXNXA

SEQ ID NO: 49 - protein, 8 aa, synthetic construct
  REGION 1..8: note = Description of sequence: LLB1 Consensus 382-389
  VARIANT 1: note = X can be either E or R
  VARIANT 2: note = X can be either S or T
  VARIANT 7: note = X can be any naturally occurring amino acid
XXYKPYXT

SEQ ID NO: 50 - protein, 8 aa, synthetic construct
  REGION 1..8: note = Description of sequence: LLB1 Consensus 421-428
  VARIANT 2: note = V can also be I
  VARIANT 3: note = X can be any naturally occurring amino acid
  VARIANT 5: note = X can be any naturally occurring amino acid
  VARIANT 7: note = X can be any naturally occurring amino acid
  VARIANT 8: note = X can be either M or L
BVXVXDXX

SEQ ID NO: 51 - skipped

SEQ ID NO: 52 - protein, 8 aa, synthetic construct
  REGION 1..8: note = Description of sequence: 380 to position 387 of a wild-type Fc polypeptide
EWESNGQP

SEQ ID NO: 53 - protein, 8 aa, synthetic construct
  REGION 1..8: note = Description of sequence: 421 to position 428 of the wild-type Fc polypeptide
NVFSCSVM

SEQ ID NO: 54 - protein, 7 aa, synthetic construct
  REGION 1..7: note = Description of sequence: 436 to position 442 of the wild-type Fc polypeptide
YTQKSLS

SEQ ID NO: 55 - protein, 630 aa, Homo sapiens
  REGION 1..630: note = Description of sequence: Human CD98hc
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 56 - protein, 13 aa, synthetic construct
  REGION 1..13: note = Description of sequence: 378-390 of wild-type Fc
AVEWESNGQPENN

SEQ ID NO: 57 - protein, 19 aa, synthetic construct
  REGION 1..19: note = Description of sequence: 422-440 of wild-type Fc
VFSCSVMHEALHNHYTQKS

SEQ ID NO: 58 - protein, 13 aa, synthetic construct
  REGION 1..13: note = Description of sequence: 42.2.19 aa 378-390
AVEWFYDDSKLTN

SEQ ID NO: 59 - protein, 13 aa, synthetic construct
  REGION 1..13: note = Description of sequence: 42.2.3-1H aa 378-390
AVEWFYGNAKETN

SEQ ID NO: 60 - protein, 13 aa, synthetic construct
  REGION 1..13: note = Description of sequence: 42.8.196 aa 378-390
AVEWFYEAQKLNN

SEQ ID NO: 61 - protein, 13 aa, synthetic construct
  REGION 1..13: note = Description of sequence: 42.8.80 aa 378-390
AVEWFSEGSKETN

SEQ ID NO: 62 - protein, 13 aa, synthetic construct
  REGION 1..13: note = Description of sequence: 42.8.15 aa 378-390
AVEWFSGAQKESN

SEQ ID NO: 63 - protein, 13 aa, synthetic construct
  REGION 1..13: note = Description of sequence: 42.8.17 aa 378-390
AVEWFSGAQKLTN

SEQ ID NO: 64 - protein, 19 aa, synthetic construct
  REGION 1..19: note = Description of sequence: 42.2.3-1H aa 422-44042.8.196 aa 422-44042.8.80 aa 422-44042.8.15 aa 422-44042.8.17 aa 422-440
LFACEVMHEALHNHYTYKL

SEQ ID NO: 65 - protein, 13 aa, synthetic construct
  REGION 1..13: note = Description of sequence: aa 378-390 consensus sequence
  VARIANT 3: note = X can be E, N, F, or Y
  VARIANT 6: note = X can be Y, S, or A
  VARIANT 7: note = X can be G, D, E, or N
  VARIANT 8: note = X can be D, G, N, or A
  VARIANT 9: note = X can be Q, S, G, A, or N
  VARIANT 10: note = X can be K, I, R, or G
  VARIANT 11: note = X can be E, L, D, or Q
  VARIANT 12: note = X can be N, T, S, or R
AVXWFXXXXXXXN

SEQ ID NO: 66 - protein, 13 aa, synthetic construct
  REGION 1..13: note = Description of sequence: aa 378-390 consensus sequence
  VARIANT 6: note = X can be either Y or S
  VARIANT 7: note = X can be G, D, or E
  VARIANT 8: note = X can be D, G, N, or A
  VARIANT 9: note = X can be Q, S, or A
  VARIANT 11: note = X can E or L
  VARIANT 12: note = X can N, T, or S
AVEWFXXXXKXXN

SEQ ID NO: 67 - protein, 19 aa, synthetic construct
  REGION 1..19: note = Description of sequence: aa 422-440 consensus sequence
  VARIANT 12: note = X can be H or E
  VARIANT 13: note = X can be N or G
LFACEVMHEALXXHYTYKL

SEQ ID NO: 68 - protein, 217 aa, synthetic construct
  REGION 1..217: note = Description of sequence: Fc consensus sequence
  VARIANT 150: note = X can be E, N, F, or Y
  VARIANT 153: note = X can be Y, S, or A
  VARIANT 154: note = X can be G, D, E, or N
  VARIANT 155: note = X can be D, G, N, or A
  VARIANT 156: note = X can be Q, S, G, A, or N
  VARIANT 157: note = X can be K, I, R, or G
  VARIANT 158: note = X can be E, L, D, or Q
  VARIANT 159: note = X can be N, T, S, or R
  VARIANT 189: note = X can be Q or P
  VARIANT 190: note = X can be G or R
  VARIANT 191: note = X can be N or G
  VARIANT 203: note = X can be H or E
  VARIANT 204: note = X can be N or G
  VARIANT 212: note = X can be S or G
  VARIANT 213: note = X can be L or E
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVXWFXXXXXXXNYKTTPPVLDSDGSFFLYSKLTVDKSRWQXXXLFACEVMHEALXXHYTYKLLXXSPGK

SEQ ID NO: 69 - protein, 217 aa, synthetic construct
  REGION 1..217: note = Description of sequence: Fc consensus sequence
  VARIANT 150: note = X can be E, N, F, or Y
  VARIANT 153: note = X can be Y, S, or A
  VARIANT 154: note = X can be G, D, E, or N
  VARIANT 155: note = X can be D, G, N, or A
  VARIANT 156: note = X can be s Q, S, G, A, or N
  VARIANT 157: note = X can be K, I, R, or G
  VARIANT 158: note = X can be E, L, D, or Q
  VARIANT 159: note = X can be N, T, S, or R
  VARIANT 189: note = X can be Q or P
  VARIANT 190: note = X can be G or R
  VARIANT 191: note = X can be N or G
  VARIANT 212: note = X can be S or G
  VARIANT 213: note = X can be L or E
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVXWFXXXXXXXNYKTTPPVLDSDGSFFLYSKLTVDKSRWQXXXLFACEVMHEALHNHYTYKLLXXSPGK

SEQ ID NO: 70 - protein, 217 aa, synthetic construct
  REGION 1..217: note = Description of sequence: Fc consensus sequence
  VARIANT 150: note = X can be E, N, F, or Y
  VARIANT 153: note = X can be Y, S, or A
  VARIANT 154: note = X can be G, D, E, or N
  VARIANT 155: note = X can be D, G, N, or A;
  VARIANT 156: note = X can be Q, S, G, A, or N
  VARIANT 157: note = X can be K, I, R, or G
  VARIANT 158: note = X can be E, L, D, or Q
  VARIANT 159: note = X can be N, T, S, or R
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVXWFXXXXXXXNYKTTPPVLDSDGSFFLYSKLTVDKSRWQQGNLFACEVMHEALHNHYTYKLLSLSPGK

SEQ ID NO: 71 - protein, 217 aa, synthetic construct
  REGION 1..217: note = Description of sequence: Fc consensus sequence
  VARIANT 153: note = X can be either Y or S
  VARIANT 154: note = X can be G, D, or E
  VARIANT 155: note = X can be D, G, N, or A
  VARIANT 156: note = X can be Q, S, or A
  VARIANT 158: note = X can be either E or L
  VARIANT 159: note = X can be N, T, or S
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFXXXXKXXNYKTTPPVLDSDGSFFLYSKLTVDKSRWQQGNLFACEVMHEALHNHYTYKLLSLSPGK

SEQ ID NO: 72 - protein, 217 aa, synthetic construct
  REGION 1..217: note = Description of sequence: 42.2.19 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYDDSKLTNYKTTPPVLDSDGSFFLYSKLTVDKSRWQPRGLFACEVMHEALHNHYTYKLLGESPGK

SEQ ID NO: 73 - protein, 217 aa, synthetic construct
  REGION 1..217: note = Description of sequence: 42.2.3-1H Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYGNAKETNYKTTPPVLDSDGSFFLYSKLTVDKSRWQQGNLFACEVMHEALHNHYTYKLLSLSPGK

SEQ ID NO: 74 - protein, 217 aa, synthetic construct
  REGION 1..217: note = Description of sequence: 42.8.196 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYEAQKLNNYKTTPPVLDSDGSFFLYSKLTVDKSRWQQGNLFACEVMHEALHNHYTYKLLSLSPGK

SEQ ID NO: 75 - protein, 217 aa, synthetic construct
  REGION 1..217: note = Description of sequence: 42.8.80 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSEGSKETNYKTTPPVLDSDGSFFLYSKLTVDKSRWQQGNLFACEVMHEALHNHYTYKLLSLSPGK

SEQ ID NO: 76 - protein, 217 aa, synthetic construct
  REGION 1..217: note = Description of sequence: 42.8.15 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSGAQKESNYKTTPPVLDSDGSFFLYSKLTVDKSRWQQGNLFACEVMHEALHNHYTYKLLSLSPGK

SEQ ID NO: 77 - protein, 217 aa, synthetic construct
  REGION 1..217: note = Description of sequence: 42.8.17 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSGAQKLTNYKTTPPVLDSDGSFFLYSKLTVDKSRWQQGNLFACEVMHEALHNHYTYKLLSLSPGK

SEQ ID NO: 78 - protein, 217 aa, synthetic construct
  REGION 1..217: note = Description of sequence: 42.2.19 Fc knob
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFYDDSKLTNYKTTPPVLDSDGSFFLYSKLTVDKSRWQPRGLFACEVMHEALHNHYTYKLLGESPGK

SEQ ID NO: 79 - protein, 217 aa, synthetic construct
  REGION 1..217: note = Description of sequence: 42.2.3-1H Fc knob
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFYGNAKETNYKTTPPVLDSDGSFFLYSKLTVDKSRWQQGNLFACEVMHEALHNHYTYKLLSLSPGK

SEQ ID NO: 80 - protein, 217 aa, synthetic construct
  REGION 1..217: note = Description of sequence: 42.8.196 Fc knob
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFYEAQKLNNYKTTPPVLDSDGSFFLYSKLTVDKSRWQQGNLFACEVMHEALHNHYTYKLLSLSPGK

SEQ ID NO: 81 - protein, 217 aa, synthetic construct
  REGION 1..217: note = Description of sequence: 42.8.80 Fc knob
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFSEGSKETNYKTTPPVLDSDGSFFLYSKLTVDKSRWQQGNLFACEVMHEALHNHYTYKLLSLSPGK

SEQ ID NO: 82 - protein, 217 aa, synthetic construct
  REGION 1..217: note = Description of sequence: 42.8.15 Fc knob
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFSGAQKESNYKTTPPVLDSDGSFFLYSKLTVDKSRWQQGNLFACEVMHEALHNHYTYKLLSLSPGK

SEQ ID NO: 83 - protein, 217 aa, synthetic construct
  REGION 1..217: note = Description of sequence: 42.8.17 Fc knob
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFSGAQKLTNYKTTPPVLDSDGSFFLYSKLTVDKSRWQQGNLFACEVMHEALHNHYTYKLLSLSPGK

SEQ ID NO: 84 - protein, 217 aa, synthetic construct
  REGION 1..217: note = Description of sequence: 42.2.19 Fc hole
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFYDDSKLTNYKTTPPVLDSDGSFFLVSKLTVDKSRWQPRGLFACEVMHEALHNHYTYKLLGESPGK

SEQ ID NO: 85 - protein, 217 aa, synthetic construct
  REGION 1..217: note = Description of sequence: 42.2.3-1H Fc hole
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFYGNAKETNYKTTPPVLDSDGSFFLVSKLTVDKSRWQQGNLFACEVMHEALHNHYTYKLLSLSPGK

SEQ ID NO: 86 - protein, 217 aa, synthetic construct
  REGION 1..217: note = Description of sequence: 42.8.196 Fc hole
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFYEAQKLNNYKTTPPVLDSDGSFFLVSKLTVDKSRWQQGNLFACEVMHEALHNHYTYKLLSLSPGK

SEQ ID NO: 87 - protein, 217 aa, synthetic construct
  REGION 1..217: note = Description of sequence: 42.8.80 Fc hole
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFSEGSKETNYKTTPPVLDSDGSFFLVSKLTVDKSRWQQGNLFACEVMHEALHNHYTYKLLSLSPGK

SEQ ID NO: 88 - protein, 217 aa, synthetic construct
  REGION 1..217: note = Description of sequence: 42.8.15 Fc hole
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFSGAQKESNYKTTPPVLDSDGSFFLVSKLTVDKSRWQQGNLFACEVMHEALHNHYTYKLLSLSPGK

SEQ ID NO: 89 - protein, 217 aa, synthetic construct
  REGION 1..217: note = Description of sequence: 42.8.17 Fc hole
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFSGAQKLTNYKTTPPVLDSDGSFFLVSKLTVDKSRWQQGNLFACEVMHEALHNHYTYKLLSLSPGK

SEQ ID NO: 90 - protein, 217 aa, synthetic construct
  REGION 1..217: note = Description of sequence: 42.2.19 Fc LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYDDSKLTNYKTTPPVLDSDGSFFLYSKLTVDKSRWQPRGLFACEVMHEALHNHYTYKLLGESPGK

SEQ ID NO: 91 - protein, 217 aa, synthetic construct
  REGION 1..217: note = Description of sequence: 42.2.3-1H Fc LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYGNAKETNYKTTPPVLDSDGSFFLYSKLTVDKSRWQQGNLFACEVMHEALHNHYTYKLLSLSPGK

SEQ ID NO: 92 - protein, 217 aa, synthetic construct
  REGION 1..217: note = Description of sequence: 42.8.196 Fc LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYEAQKLNNYKTTPPVLDSDGSFFLYSKLTVDKSRWQQGNLFACEVMHEALHNHYTYKLLSLSPGK

SEQ ID NO: 93 - protein, 217 aa, synthetic construct
  REGION 1..217: note = Description of sequence: 42.8.80 Fc LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSEGSKETNYKTTPPVLDSDGSFFLYSKLTVDKSRWQQGNLFACEVMHEALHNHYTYKLLSLSPGK

SEQ ID NO: 94 - protein, 217 aa, synthetic construct
  REGION 1..217: note = Description of sequence: 42.8.15 Fc LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSGAQKESNYKTTPPVLDSDGSFFLYSKLTVDKSRWQQGNLFACEVMHEALHNHYTYKLLSLSPGK

SEQ ID NO: 95 - protein, 217 aa, synthetic construct
  REGION 1..217: note = Description of sequence: 42.8.17 Fc LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSGAQKLTNYKTTPPVLDSDGSFFLYSKLTVDKSRWQQGNLFACEVMHEALHNHYTYKLLSLSPGK

SEQ ID NO: 96 - protein, 217 aa, synthetic construct
  REGION 1..217: note = Description of sequence: 42.2.19 Fc LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWFYDDSKLTNYKTTPPVLDSDGSFFLYSKLTVDKSRWQPRGLFACEVMHEALHNHYTYKLLGESPGK

SEQ ID NO: 97 - protein, 217 aa, synthetic construct
  REGION 1..217: note = Description of sequence: 42.2.3-1H Fc LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWFYGNAKETNYKTTPPVLDSDGSFFLYSKLTVDKSRWQQGNLFACEVMHEALHNHYTYKLLSLSPGK

SEQ ID NO: 98 - protein, 217 aa, synthetic construct
  REGION 1..217: note = Description of sequence: 42.8.196 Fc LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWFYEAQKLNNYKTTPPVLDSDGSFFLYSKLTVDKSRWQQGNLFACEVMHEALHNHYTYKLLSLSPGK

SEQ ID NO: 99 - protein, 217 aa, synthetic construct
  REGION 1..217: note = Description of sequence: 42.8.80 Fc LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWFSEGSKETNYKTTPPVLDSDGSFFLYSKLTVDKSRWQQGNLFACEVMHEALHNHYTYKLLSLSPGK

SEQ ID NO: 100 - protein, 217 aa, synthetic construct
  REGION 1..217: note = Description of sequence: 42.8.15 Fc LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWFSGAQKESNYKTTPPVLDSDGSFFLYSKLTVDKSRWQQGNLFACEVMHEALHNHYTYKLLSLSPGK

SEQ ID NO: 101 - protein, 217 aa, synthetic construct
  REGION 1..217: note = Description of sequence: 42.8.17 Fc LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWFSGAQKLTNYKTTPPVLDSDGSFFLYSKLTVDKSRWQQGNLFACEVMHEALHNHYTYKLLSLSPGK

SEQ ID NO: 102 - protein, 217 aa, synthetic construct
  REGION 1..217: note = Description of sequence: 42.2.19 Fc knob LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFYDDSKLTNYKTTPPVLDSDGSFFLYSKLTVDKSRWQPRGLFACEVMHEALHNHYTYKLLGESPGK

SEQ ID NO: 103 - protein, 217 aa, synthetic construct
  REGION 1..217: note = Description of sequence: 42.2.3-1H Fc knob LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFYGNAKETNYKTTPPVLDSDGSFFLYSKLTVDKSRWQQGNLFACEVMHEALHNHYTYKLLSLSPGK

SEQ ID NO: 104 - protein, 217 aa, synthetic construct
  REGION 1..217: note = Description of sequence: 42.8.196 Fc knob LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFYEAQKLNNYKTTPPVLDSDGSFFLYSKLTVDKSRWQQGNLFACEVMHEALHNHYTYKLLSLSPGK

SEQ ID NO: 105 - protein, 217 aa, synthetic construct
  REGION 1..217: note = Description of sequence: 42.8.80 Fc knob LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFSEGSKETNYKTTPPVLDSDGSFFLYSKLTVDKSRWQQGNLFACEVMHEALHNHYTYKLLSLSPGK

SEQ ID NO: 106 - protein, 217 aa, synthetic construct
  REGION 1..217: note = Description of sequence: 42.8.15 Fc knob LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFSGAQKESNYKTTPPVLDSDGSFFLYSKLTVDKSRWQQGNLFACEVMHEALHNHYTYKLLSLSPGK

SEQ ID NO: 107 - protein, 217 aa, synthetic construct
  REGION 1..217: note = Description of sequence: 42.8.17 Fc knob LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWFSGAQKLTNYKTTPPVLDSDGSFFLYSKLTVDKSRWQQGNLFACEVMHEALHNHYTYKLLSLSPGK

SEQ ID NO: 108 - protein, 217 aa, synthetic construct
  REGION 1..217: note = Description of sequence: 42.2.19 Fc hole LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFYDDSKLTNYKTTPPVLDSDGSFFLVSKLTVDKSRWQPRGLFACEVMHEALHNHYTYKLLGESPGK

SEQ ID NO: 109 - protein, 217 aa, synthetic construct
  REGION 1..217: note = Description of sequence: 42.2.3-1H Fc hole LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFYGNAKETNYKTTPPVLDSDGSFFLVSKLTVDKSRWQQGNLFACEVMHEALHNHYTYKLLSLSPGK

SEQ ID NO: 110 - protein, 217 aa, synthetic construct
  REGION 1..217: note = Description of sequence: 42.8.196 Fc hole LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFYEAQKLNNYKTTPPVLDSDGSFFLVSKLTVDKSRWQQGNLFACEVMHEALHNHYTYKLLSLSPGK

SEQ ID NO: 111 - protein, 217 aa, synthetic construct
  REGION 1..217: note = Description of sequence: 42.8.80 Fc hole LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFSEGSKETNYKTTPPVLDSDGSFFLVSKLTVDKSRWQQGNLFACEVMHEALHNHYTYKLLSLSPGK

SEQ ID NO: 112 - protein, 217 aa, synthetic construct
  REGION 1..217: note = Description of sequence: 42.8.15 Fc hole LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFSGAQKESNYKTTPPVLDSDGSFFLVSKLTVDKSRWQQGNLFACEVMHEALHNHYTYKLLSLSPGK

SEQ ID NO: 113 - protein, 217 aa, synthetic construct
  REGION 1..217: note = Description of sequence: 42.8.17 Fc hole LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWFSGAQKLTNYKTTPPVLDSDGSFFLVSKLTVDKSRWQQGNLFACEVMHEALHNHYTYKLLSLSPGK

SEQ ID NO: 114 - protein, 217 aa, synthetic construct
  REGION 1..217: note = Description of sequence: 42.2.19 Fc knob LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWFYDDSKLTNYKTTPPVLDSDGSFFLYSKLTVDKSRWQPRGLFACEVMHEALHNHYTYKLLGESPGK

SEQ ID NO: 115 - protein, 217 aa, synthetic construct
  REGION 1..217: note = Description of sequence: 42.2.3-1H Fc knob LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWFYGNAKETNYKTTPPVLDSDGSFFLYSKLTVDKSRWQQGNLFACEVMHEALHNHYTYKLLSLSPGK

SEQ ID NO: 116 - protein, 217 aa, synthetic construct
  REGION 1..217: note = Description of sequence: 42.8.196 Fc knob LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWFYEAQKLNNYKTTPPVLDSDGSFFLYSKLTVDKSRWQQGNLFACEVMHEALHNHYTYKLLSLSPGK

SEQ ID NO: 117 - protein, 217 aa, synthetic construct
  REGION 1..217: note = Description of sequence: 42.8.80 Fc knob LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWFSEGSKETNYKTTPPVLDSDGSFFLYSKLTVDKSRWQQGNLFACEVMHEALHNHYTYKLLSLSPGK

SEQ ID NO: 118 - protein, 217 aa, synthetic construct
  REGION 1..217: note = Description of sequence: 42.8.15 Fc knob LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWFSGAQKESNYKTTPPVLDSDGSFFLYSKLTVDKSRWQQGNLFACEVMHEALHNHYTYKLLSLSPGK

SEQ ID NO: 119 - protein, 217 aa, synthetic construct
  REGION 1..217: note = Description of sequence: 42.8.17 Fc knob LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWFSGAQKLTNYKTTPPVLDSDGSFFLYSKLTVDKSRWQQGNLFACEVMHEALHNHYTYKLLSLSPGK

SEQ ID NO: 120 - protein, 226 aa, synthetic construct
  REGION 1..226: note = Description of sequence: 42.2.19 Fc hole LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTSLSCLVCAVKGFYPSDIAVEWFYDDSKLTNYKTTPPVLDSDGSFFLYSLVSKLTVDKSRWQPRGLFACEVMHEALHNHYTYKLLGESPGK

SEQ ID NO: 121 - protein, 226 aa, synthetic construct
  REGION 1..226: note = Description of sequence: 42.2.3-1H Fc hole LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTSLSCLVCAVKGFYPSDIAVEWFYGNAKETNYKTTPPVLDSDGSFFLYSLVSKLTVDKSRWQQGNLFACEVMHEALHNHYTYKLLSLSPGK

SEQ ID NO: 122 - protein, 226 aa, synthetic construct
  REGION 1..226: note = Description of sequence: 42.8.196 Fc hole LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTSLSCLVCAVKGFYPSDIAVEWFYEAQKLNNYKTTPPVLDSDGSFFLYSLVSKLTVDKSRWQQGNLFACEVMHEALHNHYTYKLLSLSPGK

SEQ ID NO: 123 - protein, 226 aa, synthetic construct
  REGION 1..226: note = Description of sequence: 42.8.80 Fc hole LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTSLSCLVCAVKGFYPSDIAVEWFSEGSKETNYKTTPPVLDSDGSFFLYSLVSKLTVDKSRWQQGNLFACEVMHEALHNHYTYKLLSLSPGK

SEQ ID NO: 124 - protein, 226 aa, synthetic construct
  REGION 1..226: note = Description of sequence: 42.8.15 Fc hole LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTSLSCLVCAVKGFYPSDIAVEWFSGAQKESNYKTTPPVLDSDGSFFLYSLVSKLTVDKSRWQQGNLFACEVMHEALHNHYTYKLLSLSPGK

SEQ ID NO: 125 - protein, 220 aa, synthetic construct
  REGION 1..220: note = Description of sequence: 42.8.17 Fc hole LALAPG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSTCALVKGFYPSDIAVEWFSGAQKLTNYKTTPPVLDSDGSFFLVYSKLTVDKSRWQQGNLFACEVMHEALHNHYTYKLLSLSPGK

SEQ ID NO: 126 - protein, 217 aa, synthetic construct
  REGION 1..217: note = Description of sequence: CH3C.35.21
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVWWESYGTEWSSYKTTPPVLDSDGSFFLYSKLTVTKEEWQQGFVFSCSVMHEALHNHYTQKSLSLSPGK

SEQ ID NO: 127 - protein, 760 aa, Homo sapiens
  REGION 1..760: note = Description of sequence: Human transferrin receptor protein 1 (TFR1)
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 128 - protein, 760 aa, Macaca fascicularis
  REGION 1..760: note = Description of sequence: Cyno TfR
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 129 - protein, 181 aa, Homo sapiens
  REGION 1..181: note = Description of sequence: Human TfR apical domain
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 130 - protein, 181 aa, Macaca fascicularis
  REGION 1..181: note = Description of sequence: Cynomolgus TfR apical domain
NSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVS

SEQ ID NO: 131 - protein, 159 aa, Homo sapiens
  REGION 1..159: note = Description of sequence: Loop-truncated human TfR apical domain displayed on phage
SSGLPNIPVQTISRAAAEKLFGNMEGDCPSDWKTDSTCRMVTSESKNVKLTVSNDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSGP

SEQ ID NO: 132 - protein, 159 aa, Macaca fascicularis
  REGION 1..159: note = Description of sequence: Loop-truncated cynomolgus TfR apical domain displayed on phage
SSGLPNIPVQTISRAAAEKLFGNMEGDCPSDWKTDSTCKMVTSENKSVKLTVSNDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSGP

SEQ ID NO: 133 - protein, 186 aa, synthetic construct
  REGION 1..186: note = Description of sequence: His-tagged permutated TfR apical domain
MGWSCIILFLVATATGAYAGTSSGLPNIPVQTISRAAAEKLFGNMEGDCPSDWKTDSTCRMVTSESKNVKLTVSNDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSASHHHHHH

SEQ ID NO: 134 - protein, 529 aa, Homo sapiens
  REGION 1..529: note = Description of sequence: Uniprot P08195-2|4F2_HUMAN Isoform 2 of 4F2 cell-surface antigen heavy chain
MSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 135 - protein, 421 aa, Homo sapiens
  REGION 1..421: note = Description of sequence: hCD98hc ECD: positions 109-520 of Uniprot P08195-2|4F2_HUMAN Isoform 2 of 4F2 cell-surface antigen heavy chain
CRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 136 - protein, 421 aa, Homo sapiens
  REGION 1..421: note = Description of sequence: hCD98hc ECD for crystal structures: positions 109-520 of Uniprot P08195-2-4F2_HUMAN Isoform 2 of 4F2 cell-surface antigen heavy chain with G at position 109 and S at position 110 in crystal structures
GSELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 137 - protein, 217 aa, synthetic construct
  REGION 1..217: note = Description of sequence: Clone 1-321
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIHVEWGSLVQPENNYKTTPPVLDSDGSFFLYSKLTVDKSRWQQGNIFPCIVMHEALHNHYTIKTLSLSPGK

SEQ ID NO: 138 - protein, 217 aa, synthetic construct
  REGION 1..217: note = Description of sequence: Clone 1-321_M428L
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIHVEWGSLVQPENNYKTTPPVLDSDGSFFLYSKLTVDKSRWQQGNIFPCIVLHEALHNHYTIKTLSLSPGK

SEQ ID NO: 139 - protein, 12 aa, synthetic construct
  REGION 1..12: note = Description of sequence: aa 378-390 consensus sequence
  VARIANT 3: note = X can be E, N, F, or Y
  VARIANT 6: note = X can be G, D, E, or N
  VARIANT 7: note = X can be D, G, N, or A
  VARIANT 8: note = X can be Q, S, G, A, or N
  VARIANT 9: note = X can be K, I, R, or G
  VARIANT 10: note = X can be E, L, D, or Q
  VARIANT 11: note = X can be N, T, S, or R
AVXWFXXXXXXN

SEQ ID NO: 140 - protein, 216 aa, synthetic construct
  REGION 1..216: note = Description of sequence: Fc consensus sequence
  VARIANT 150: note = X can be E, N, F, or Y
  VARIANT 153: note = X can be G, D, E, or N
  VARIANT 154: note = X can be D, G, N, or A
  VARIANT 155: note = X can be Q, S, G, A, or N
  VARIANT 156: note = X can be K, I, R, or G
  VARIANT 157: note = X can be E, L, D, or Q
  VARIANT 158: note = X can be N, T, S, or R
  VARIANT 188: note = X can be Q or P
  VARIANT 189: note = X can be G or R
  VARIANT 190: note = X can be N or G
  VARIANT 202: note = X can be H or E
  VARIANT 203: note = X can be N or G
  VARIANT 211: note = X can be S or G
  VARIANT 212: note = X can be L or E
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVXWFXXXXXXNYKTTPPVLDSDGSFFLYSKLTVDKSRWQXXXLFACEVMHEALXXHYTYKLLXXSPGK

SEQ ID NO: 141 - protein, 216 aa, synthetic construct
  REGION 1..216: note = Description of sequence: Fc consensus sequence
  VARIANT 150: note = X can be E, N, F, or Y
  VARIANT 153: note = X can be G, D, E, or N
  VARIANT 154: note = X can be D, G, N, or A
  VARIANT 155: note = X can be s Q, S, G, A, or N
  VARIANT 156: note = X can be K, I, R, or G
  VARIANT 157: note = X can be E, L, D, or Q
  VARIANT 158: note = X can be N, T, S, or R
  VARIANT 188: note = X can be Q or P
  VARIANT 189: note = X can be G or R
  VARIANT 190: note = X can be N or G
  VARIANT 212: note = X can be S or G
  VARIANT 213: note = X can be L or E
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVXWFXXXXXXNYYKTTPPVLDSDGSFFYSKLTVDKSRWQXXXLFACEVMHEALHNHYTYKLLXXSPGK

SEQ ID NO: 142 - protein, 216 aa, synthetic construct
  REGION 1..216: note = Description of sequence: Fc consensus sequence
  VARIANT 150: note = X can be E, N, F, or Y
  VARIANT 153: note = X can be G, D, E, or N
  VARIANT 154: note = X can be D, G, N, or A;
  VARIANT 155: note = X can be Q, S, G, A, or N
  VARIANT 156: note = X can be K, I, R, or G
  VARIANT 157: note = X can be E, L, D, or Q
  VARIANT 158: note = X can be N, T, S, or R
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVXWFXXXXXXNYKTTPPVLDSDGSFFLYSKLTVDKSRWQQGNLFACEVMHEALHNHYTYKLLSLSPGK